(19) United States
(12) Patent Application Publication
(10) Pub. No.: US 2025/0333767 A1
(43) Pub. Date: Oct. 30, 2025

(54) ENGINEERED CLASS 2 TYPE V CRISPR SYSTEMS

(71) Applicant: Scribe Therapeutics Inc., Alameda, CA (US)

(72) Inventors: Wenyuan ZHOU, Dublin, CA (US); Fred DEITER, Alameda, CA (US); Manuel MOHR, Berkeley, CA (US); Benjamin OAKES, El Cerrito, CA (US); Sean HIGGINS, Alameda, CA (US); Sarah DENNY, San Francisco, CA (US); Gayathri VIJAYAKUMAR, Oakland, CA (US); Trent GOMBERG, San Diego, CA (US); Addison WRIGHT, El Cerrito, CA (US)

(21) Appl. No.: 18/869,765

(22) PCT Filed: Jun. 1, 2023
(86) PCT No.: PCT/US2023/067791

§ 371 Date: Nov. 26, 2024

Related U.S. Application Data
(60) Provisional application No. 63/348,413, filed on Jun. 2, 2022.
(60) Provisional application No. 63/350,400, filed on Jun. 8, 2022.
(60) Provisional application No. 63/350,770, filed on Jun. 9, 2022.

(51) Int. Cl. C12N 15/90; C12N 9/22; C12N 15/11

(52) U.S. Cl. CPC C12N 15/907; C12N 9/226; C12N 15/11; C12N 2310/20; C12N 2310/531

(57) ABSTRACT

Provided herein are systems of engineered Class 2, Type V nucleases and guide ribonucleic acid scaffolds useful for the editing of target nucleic acids. Also provided are methods of making and using such systems to modify nucleic acids.

### CROSS-REFERENCE TO RELATED APPLICATION

This application claims priority to U.S. provisional patent application Nos. 63/348,413, filed on Jun. 2, 2022, 63/350,400, filed on Jun. 8, 2022, and 63/350,770, filed on Jun. 9, 2022, the contents of each of which are incorporated by reference in their entirety.

### INCORPORATION BY REFERENCE OF SEQUENCE LISTING

The contents of the electronic sequence listing (SCRB_041_03WO_SeqList_ST26.xml; Size: 90,175,867 bytes; and Date of Creation: May 23, 2023) are herein incorporated by reference in their entirety.

### BACKGROUND

The CRISPR-Cas systems of bacteria and archaea confer a form of acquired immunity against phage and viruses. Intensive research over the past decade has uncovered the biochemistry of these systems. CRISPR-Cas systems consist of Cas proteins, which are involved in acquisition, targeting and cleavage of foreign DNA or RNA, and a CRISPR array, which includes direct repeats flanking short spacer sequences that guide Cas proteins to their targets. Class 2 CRISPR-Cas are streamlined versions in which a single Cas protein bound to RNA is responsible for binding to and cleavage of a targeted sequence. The programmable nature of these minimal systems has facilitated their use as a versatile technology that is revolutionizing the field of genome manipulation.

To date, only a few Class 2 CRISPR/Cas systems have been discovered that have been widely used. Of these, Type V are unique in that they utilize a single unified RuvC-like endonuclease (RuvC) domain that recognizes 5′ PAM sequences that are different from the 3′ PAM sequences recognized by Cas9, and form a staggered cleavage in the target nucleic acid with 5, 7, or 10 nt 5′ overhangs (Yang et al., PAM-dependent target DNA recognition and cleavage by C2c1 CRISPR-Cas endonuclease. Cell 167:1814 (2016)). However, wild-type Type V Cas nuclease and guide sequences have low editing efficiency. Thus, there is a need in the art for additional Class 2, Type V CRISPR/Cas systems (e.g., Cas protein plus guide RNA combinations) that have been optimized and offer improvements over earlier generation systems for utilization in a variety of therapeutic, diagnostic, and research applications.

### SUMMARY

The present disclosure relates to systems of engineered CasX proteins and engineered guide ribonucleic acid scaffolds (ERS) with linked targeting sequences used to modify a target nucleic acid of a gene in eukaryotic cells. In some embodiments, the present disclosure provides engineered CasX proteins comprising one or more, or multiple modifications relative to one or more domains of a CasX protein from which it was derived. These engineered CasX exhibit one or more improved characteristics as compared to a reference CasX or the CasX variant from which it was derived, and the engineered CasX retains the ability to form a ribonucleoprotein (RNP) complex with an ERS and retains nuclease activity.

In another aspect, the present disclosure provides engineered guide ribonucleic acid scaffolds (ERS), including single-guide compositions, capable of binding a Class 2, Type V protein, including the engineered CasX of the disclosure, wherein the ERS comprise one or more, or multiple modifications in one or more regions compared to a parental gRNA; e.g., a reference gRNA or a gRNA variant. In some embodiments, the modified regions of the scaffold of the gRNA include one or more of: (a) the 5′ end of the scaffold; (b) the extended stem; (c) the scaffold stem; (d) the triplex; (e) the triplex loop; and (f) the pseudoknot stem.

In some embodiments, the present disclosure provides systems of gene editing pairs comprising the engineered CasX proteins and ERS of any of the embodiments described herein, wherein the gene editing pair exhibits at least one improved characteristic as compared to a gene editing pair of a CasX and gRNA from which the engineered CasX proteins and ERS were derived.

In some embodiments, the present disclosure provides polynucleotides and vectors encoding the engineered CasX proteins, ERS and gene editing pairs described herein. In some embodiments, the vectors are viral vectors such as an Adeno-Associated Viral (AAV) vector. In other embodiments, the vectors are CasX delivery particles (XDP) that comprise RNPs of the gene editing pairs.

In some embodiments, the present disclosure provides methods of making the engineered CasX proteins. In other embodiments, the disclosure provides methods of making the ERS.

In some embodiments, the present disclosure provides kits comprising the polynucleotides, vectors, engineered CasX proteins, ERS and gene editing pairs, and LNP compositions described herein.

In some embodiments, the present disclosure provides methods of editing a target nucleic acid, comprising contacting the target nucleic acid with the engineered CasX protein and ERS embodiments described herein, wherein the contacting results in editing or modification of the target nucleic acid.

In some embodiments, the present disclosure provides methods of editing a target nucleic acid in a population of cells, comprising contacting the cells with one or more of the gene editing pairs described herein, wherein the contacting results in editing or modification of the target nucleic acid in the population of cells.

In another aspect, provided herein are gene editing pairs, compositions comprising gene editing pairs, or vectors comprising or encoding gene editing pairs, for use in a method of treatment, wherein the method comprises editing or modifying a target nucleic acid; optionally wherein the editing occurs in a subject having a mutation in an allele of a gene wherein the mutation causes a disease or disorder in the subject, preferably wherein the editing changes the mutation to a wild type allele of the gene or knocks down or knocks out an allele of a gene causing a disease or disorder in the subject.

In another aspect, the present disclosure provides compositions of engineered CasX, ERS, and gene editing pairs for use in the manufacture of a medicament for use in the treatment of a subject with a disease.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a graph illustrating the results of the CcdB bacterial selection assay to determine the true fitness values (represented as log 2 enrichment scores) for the new CasX variants (top graph), which were designed via machine learning to contain the indicated number of single mutations relative to CasX 515, and for the randomly mutated CasX molecules (bottom graph), as described in Example 1.

FIG. 2 is a graph illustrating the results of the stringent CcdB bacterial selection assay to determine the true fitness values (represented as log 2 enrichment scores) for the new machine-learning derived CasX variants (top graph) and for the randomly mutated CasX molecules (bottom graph), as described in Example 1.

FIG. 3A is a bar graph showing the average on-target editing efficiency for the indicated CasX variants for two biological replicates, as described in Example 2. Standard error of the mean was also determined and illustrated.

FIG. 3B is a bar graph showing the average off-target editing efficiency for the indicated CasX variants for two biological replicates, as described in Example 2. Standard error of the mean was also determined and illustrated.

FIG. 4 is a bar graph showing the average on-target editing efficiency for the indicated CasX variants across a series of four different PAM sequences, as described in Example 2. Standard error of the mean was also determined and illustrated.

FIG. 5 is a bar graph showing results from the CcdB survival assay, plotting mean log 2 enrichment values as an assessment for nuclease activity for the indicated CasX protein variants, as described in Example 2. Standard error of the mean was also determined and illustrated.

FIG. 6A is a boxplot showing the average on-target editing activity for selected CasX variants in the PASS assay, as described in Example 3. Forty on-target, TTC PAM spacer-targets are shown, where each was averaged across six replicates.

FIG. 6B is a boxplot showing the average off-target editing activity for selected CasX variants in the PASS assay, as described in Example 3. Eighty off-target, TTC PAM spacer-targets are shown, where each was averaged across six replicates.

FIG. 7A is a pointplot showing CasX 491 average editing activity and 95% estimated confidence intervals for TTC-PAM spacers and their editing rates at either perfectly complementary on-target sites or mismatched off-target sites, as described in Example 3. On-target editing rates are plotted as squares, while off-target editing rates are plotted as circles. Regions highlighted in gray are defined as allele-specific, where the on-target editing rate is >20%, and the off-target editing rate is <20% of the on-target editing rate.

FIG. 7B is a pointplot showing CasX 515 average editing activity and 95% estimated confidence intervals for TTC-PAM spacers and their editing rates at either perfectly complementary on-target sites or mismatched off-target sites, as described in Example 3.

FIG. 7C is a pointplot showing CasX 812 average editing activity and 95% estimated confidence intervals for TTC-PAM spacers and their editing rates at either perfectly complementary on-target sites or mismatched off-target sites, as described in Example 3.

FIG. 8A is a schematic illustrating versions 1-3 of chemical modifications made to gRNA scaffold 235, as described in Example 8. Structural motifs are highlighted. Standard ribonucleotides are depicted as open circles, and 2′OMe-modified ribonucleotides are depicted as black circles. Phosphorothioate bonds are indicated with * below or beside the bond. For the v2 profile, the addition of three 3′ uracils (3′UUU) is annotated with “U”s in the relevant circles.

FIG. 8B is a schematic illustrating versions 4-6 of chemical modifications made to gRNA scaffold 235, as described in Example 8. Structural motifs are highlighted. Standard ribonucleotides are depicted as open circles, and 2′OMe-modified ribonucleotides are depicted as black circles. Phosphorothioate bonds are indicated with * below or beside the bond.

FIG. 9 is a plot illustrating the quantification of percent knockout of B2M in HepG2 cells co-transfected with 100 ng of CasX 491 mRNA and with the indicated doses of end-modified (vi) or unmodified (v0) B2M-targeting gRNAs with spacer 7.37, as described in Example 8. Editing level was determined by flow cytometry as the population of cells with loss of surface presentation of the HLA complex due to successful editing at the B2M locus.

FIG. 10 is a schematic illustrating versions 7-9 of chemical modifications made to ERS 316, as described in Example 8. Structural motifs are highlighted. Standard ribonucleotides are depicted as open circles, and 2′OMe-modified ribonucleotides are depicted as black circles. Phosphorothioate bonds are indicated with * below or beside the bond.

FIG. 11A is a schematic of gRNA scaffold 174, as described in Example 8. Structural motifs are highlighted.

FIG. 11B is a schematic of gRNA scaffold 235, as described in Example 8. Highlighted structural motifs are the same as in FIG. 6A. The differences between scaffold 174 and scaffold 235 lie in the extended stem motif and several single-nucleotide changes (indicated with asterisks). ERS 316 maintains the shorter extended stem from scaffold 174 but harbors the four substitutions found in scaffold 235.

FIG. 11C is a schematic of ERS 316, as described in Example 8. Highlighted structural motifs are the same as in FIG. 6A. ERS 316 maintains the shorter extended stem from scaffold 174 (FIG. 6A) but harbors the four substitutions found in scaffold 235 (FIG. 6B).

FIG. 12 is a plot displaying a correlation between indel rate (depicted as edit fraction) at the PCSK9 locus as measured by NGS (x-axis) and secreted PCSK9 levels (ng/mL) detected by enzyme-linked immunosorbent assay (ELISA) (y-axis) in HepG2 cells lipofected with CasX 491 mRNA and PCSK9-targeting gRNAs containing the indicated scaffold variant and spacer combination, as described in Example 8.

FIG. 13A is a plot depicting the results of an editing assay measured as indel rate detected by NGS at the human B2M locus in HepG2 cells treated with the indicated doses of LNPs formulated with CasX 491 mRNA and the indicated B2M-targeting gRNA, as described in Example 8.

FIG. 13B is a plot illustrating the quantification of percent knockout of B2M in HepG2 cells treated with the indicated doses of LNPs formulated with CasX 491 mRNA and the indicated B2M-targeting gRNA, as described in Example 8. Editing level was determined by flow cytometry as population of cells that did not have surface presentation of the HLA complex due to successful editing at the B2M locus.

FIG. 14A is a plot depicting the results of an editing assay measured as indel rate detected by NGS at the mouse ROSA26 locus in Hepa1-6 cells treated with the indicated doses of LNPs formulated with CasX 676 mRNA #2 and the indicated ROSA26-targeting gRNA with either the v1 or v5 modification profile, as described in Example 8.

FIG. 14B is a plot illustrating the quantification of percent editing measured as indel rate detected by NGS at the ROSA26 locus in mice treated with LNPs formulated with CasX 676 mRNA #2 and the indicated chemically-modified ROSA26-targeting gRNA, as described in Example 8.

FIG. 15 is a bar graph showing the results of the editing assay measured as indel rate detected by NGS as the mouse PCSK9 locus in mice treated with LNPs formulated with CasX 676 mRNA #1 and the indicated chemically-modified PCSK9-targeting gRNA, as described in Example 8. Untreated mice served as experimental control.

FIG. 16A is a schematic illustrating versions 1-3 of chemical modifications made to ERS 316, as described in Example 8. Structural motifs are highlighted. Standard ribonucleotides are depicted as open circles, and 2′OMe-modified ribonucleotides are depicted as black circles. Phosphorothioate bonds are indicated with * below or beside the bond.

FIG. 16B is a schematic illustrating versions 4-6 of chemical modifications made to ERS 316, as described in Example 8. Structural motifs are highlighted. Standard ribonucleotides are depicted as open circles, and 2′OMe-modified ribonucleotides are depicted as black circles. Phosphorothioate bonds are indicated with * below or beside the bond.

FIG. 17A is a diagram of the secondary structure of guide RNA scaffold 235, noting the regions with CpG motifs, as described in Example 9. CpG motifs in (1) the pseudoknot stem, (2) the scaffold stem, (3) the extended stem bubble, (4) the extended step, and (5) the extended stem loop are labeled on the structure.

FIG. 17B is a diagram of the CpG-reducing mutations that were introduced into each of the five regions in the coding sequence of the guide RNA scaffold, as described in Example 9.

FIG. 18 provides the results of an editing experiment in which AAV vectors with various CpG-reduced or CpG-depleted guide RNA scaffolds were used to edit the B2M locus in induced neurons, as described in Example 9. The AAV vectors were administered at a multiplicity of infection (MOI) of 4e3. The bars show the mean±the SD of two replicates per sample. “No Tx” indicates a non-transduced control, and “NT” indicates a control with a non-targeting spacer.

FIG. 19 provides the results of an editing experiment in which AAV vectors with various CpG-reduced or CpG-depleted guide RNA scaffolds were used to edit the B2M locus in induced neurons, as described in Example 9. The AAV vectors were administered at an MOI of 3e3. The bars show the mean±the SD of two replicates per sample. “No Tx” indicates a non-transduced control.

FIG. 20 provides the results of an editing experiment in which AAV vectors with various CpG-reduced or CpG-depleted guide RNA scaffolds were used to edit the B2M locus in induced neurons, as described in Example 9. The AAV vectors were administered at an MOI of 1e3. The bars show the mean±the SD of two replicates per sample. “No Tx” indicates a non-transduced control.

FIG. 21 provides the results of an editing experiment in which AAV vectors with various CpG-reduced or CpG-depleted guide RNA scaffolds were used to edit the B2M locus in induced neurons, as described in Example 9. The AAV vectors were administered at an MOI of 3e2. The bars show the mean±the SD of two replicates per sample. “No Tx” indicates a non-transduced control.

FIG. 22 is a bar graph showing the quantification of percent knockout of B2M in HEK293 cells transfected with CpG-depleted AAV plasmids containing the indicated gRNA scaffolds with spacer 7.37, as described in Example 10. The dotted line annotates the ˜41% transfection efficiency.

FIG. 23A is a bar plot showing percent editing at the AAVS1 locus in human iNs transduced with AAVs expressing the CasX:gRNA system using the indicated gRNA scaffolds (AAV construct ID #262-274) at the MOI of 3E4 vg/cell, as described in Example 10.

FIG. 23B is a bar plot showing percent editing at the AAVS1 locus in human iNs transduced with AAVs expressing the CasX:gRNA system using the indicated gRNA scaffolds (AAV construct ID #262-274) at the MOI of 1E4 vg/cell, as described in Example 10.

FIG. 23C is a bar plot showing percent editing at the AAVS1 locus in human iNs transduced with AAVs expressing the CasX:gRNA system using the indicated gRNA scaffolds (AAV construct ID #262-274) at the MOI of 3E3 vg/cell, as described in Example 10.

FIG. 24A is a bar plot showing the quantification of percent knockout of B2M in HEK293 cells transfected with CpG-depleted AAV plasmids containing the indicated gRNA scaffolds with spacer 7.37 (AAV construct ID #275-289) at the MOI of 1E4 vg/cell, as described in Example 10.

FIG. 24B is a bar plot showing the quantification of percent knockout of B2M in HEK293 cells transfected with CpG-depleted AAV plasmids containing the indicated gRNA scaffolds with spacer 7.37 (AAV construct ID #275-289) at the MOI of 3E3 vg/cell, as described in Example 10.

FIG. 24C is a bar plot showing the quantification of percent knockout of B2M in HEK293 cells transfected with CpG-depleted AAV plasmids containing the indicated gRNA scaffolds with spacer 7.37 (AAV construct ID #275-289) at the MOI of 1E3 vg/cell, as described in Example 10.

FIG. 25 is a diagram of the secondary structure of guide RNA scaffold 316, noting the regions and domains in which mutations were designed for screening in a library, as described in Example 11. The (1) 5′ end, (2) pseudoknot stem, (3) triplex loop, (4) triplex (including adjacent sequence between the extended stem and the start of annotated triplex), (5) scaffold stem (including adjacent sequences from the end of pseudoknot and start of extended stem), and (6) extended stem are labeled on the structure.

FIG. 26 shows the results of an editing experiment in which HEK293T cells were transduced with lentiviral particles expressing CasX 515 and a gRNA made up of either scaffold 174, scaffold 235, ERS 316, ERS 382, or ERS 392 targeting the B2M locus or a non-targeting (“NT”) control, as described in Example 12. The lentiviruses were transduced at an MOI of 0.1. The bars show the mean of three samples, and the error bars represent the standard error of the mean (SEM).

FIG. 27 shows the results of an editing experiment in which HEK293T cells were transduced with lentiviral particles expressing CasX 515 and a gRNA made up of either scaffold 174, scaffold 235, ERS 316, ERS 382, or ERS 392 targeting the B2M locus or a non-targeting (“NT”) control, as described in Example 12. The lentiviruses were transduced at a MOI of 0.05. The bars show the mean of three samples, and the error bars represent the SEM.

FIG. 28 is a western blot showing the levels of CasX expression (top western blot) in HEK293 cells transfected with AAV plasmids containing a CpG+ CasX 515 sequence (lane 1) or CpG− v1 CasX 515 sequence (lanes 2-3), as described in Example 13. Lysate from untransfected HEK293 cells were used as a ‘no plasmid’ control (lane 4). The bottom western blot shows the total protein loading control. Three technical replicates are shown.

### DETAILED DESCRIPTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Unless otherwise defined, all technical and scientific terms used herein have the same meaning as commonly understood by one of ordinary skill in the art to which this invention belongs. Although methods and materials similar or equivalent to those described herein can be used in the practice or testing of the present embodiments, suitable methods and materials are described below. In case of conflict, the patent specification, including definitions, will control. In addition, the materials, methods, and examples are illustrative only and not intended to be limiting. Numerous variations, changes, and substitutions will now occur to those skilled in the art without departing from the invention.

### Definitions

The terms “polynucleotide” and “nucleic acid,” used interchangeably herein, refer to a polymeric form of nucleotides of any length, either ribonucleotides or deoxyribonucleotides. Thus, terms “polynucleotide” and “nucleic acid” encompass single-stranded DNA; double-stranded DNA; multi-stranded DNA; single-stranded RNA; double-stranded RNA; multi-stranded RNA; genomic DNA; cDNA; DNA-RNA hybrids; and a polymer comprising purine and pyrimidine bases or other natural, chemically or biochemically modified, non-natural, or derivatized nucleotide bases.

“Hybridizable” or “complementary” are used interchangeably to mean that a nucleic acid (e.g., RNA, DNA) comprises a sequence of nucleotides that enables it to non-covalently bind, i.e., form Watson-Crick base pairs and/or G/U base pairs, “anneal”, or “hybridize,” to another nucleic acid in a sequence-specific, antiparallel, manner (i.e., a nucleic acid specifically binds to a complementary nucleic acid) under the appropriate in vitro and/or in vivo conditions of temperature and solution ionic strength. It is understood that the sequence of a polynucleotide need not be 100% complementary to that of its target nucleic acid sequence to be specifically hybridizable; it can have at least about 70%, at least about 80%, or at least about 90%, or at least about 95% sequence identity and still hybridize to the target nucleic acid sequence. Moreover, a polynucleotide may hybridize over one or more segments such that intervening or adjacent segments are not involved in the hybridization event (e.g., a loop structure or hairpin structure, a ‘bulge’, and the like).

A “gene,” for the purposes of the present disclosure, includes a DNA region encoding a gene product (e.g., a protein, RNA), as well as all DNA regions which regulate the production of the gene product, whether or not such regulatory sequences are adjacent to coding and/or transcribed sequences. Accordingly, a gene may include regulatory element sequences including, but not necessarily limited to, promoter sequences, terminators, translational regulatory sequences such as ribosome binding sites and internal ribosome entry sites, enhancers, silencers, insulators, boundary elements, replication origins, matrix attachment sites and locus control regions. Coding sequences encode a gene product upon transcription or transcription and translation; the coding sequences of the disclosure may comprise fragments and need not contain a full-length open reading frame. A gene can include both the strand that is transcribed as well as the complementary strand containing the anticodons.

The term “downstream” refers to a nucleotide sequence that is located 3′ to a reference nucleotide sequence. In certain embodiments, downstream nucleotide sequences relate to sequences that follow the starting point of transcription. For example, the translation initiation codon of a gene is located downstream of the start site of transcription.

The term “upstream” refers to a nucleotide sequence that is located 5′ to a reference nucleotide sequence. In certain embodiments, upstream nucleotide sequences relate to sequences that are located on the 5′ side of a coding region or starting point of transcription. For example, most promoters are located upstream of the start site of transcription.

The term “adjacent to” with respect to polynucleotide or amino acid sequences refers to sequences that are next to, or adjoining each other in a polynucleotide or polypeptide. The skilled artisan will appreciate that two sequences can be considered to be adjacent to each other and still encompass a limited amount of intervening sequence, e.g., 1, 2, 3, 4, 5, 6, 7, 8, 9 or 10 nucleotides or amino acids.

The term “regulatory element” is used interchangeably herein with the term “regulatory sequence,” and is intended to include promoters, enhancers, and other expression regulatory elements. It will be understood that the choice of the appropriate regulatory element will depend on the encoded component to be expressed (e.g., protein or RNA) or whether the nucleic acid comprises multiple components that require different polymerases or are not intended to be expressed as a fusion protein.

The term “accessory element” is used interchangeably herein with the term “accessory sequence,” and is intended to include, inter alia, polyadenylation signals (poly(A) signal), enhancer elements, introns, posttranscriptional regulatory elements (PTREs), nuclear localization signals (NLS), deaminases, DNA glycosylase inhibitors, additional promoters, factors that stimulate CRISPR-mediated homology-directed repair (e.g. in cis or in trans), activators or repressors of transcription, self-cleaving sequences, and fusion domains, for example a fusion domain fused to an engineered CasX protein. It will be understood that the choice of the appropriate accessory element or elements will depend on the encoded component to be expressed (e.g., protein or RNA) or whether the nucleic acid comprises multiple components that require different polymerases or are not intended to be expressed as a fusion protein.

The term “promoter” refers to a DNA sequence that contains a transcription start site and additional sequences to facilitate polymerase binding and transcription. Exemplary eukaryotic promoters include elements such as a TATA box, and/or B recognition element (BRE) and assists or promotes the transcription and expression of an associated transcribable polynucleotide sequence and/or gene (or transgene). A promoter can be synthetically produced or can be derived from a known or naturally occurring promoter sequence or another promoter sequence. A promoter can also include a chimeric promoter comprising a combination of two or more heterologous sequences to confer certain properties. A promoter of the present disclosure can include variants of promoter sequences that are similar in composition, but not identical to, other promoter sequence(s) known or provided herein. A promoter can be classified according to criteria relating to the pattern of expression of an associated coding or transcribable sequence or gene operably linked to the promoter, such as constitutive, developmental, tissue-specific, inducible, etc. A promoter can also be classified according to its strength. As used in the context of a promoter, “strength” refers to the rate of transcription of the gene controlled by the promoter. A “strong” promoter means the rate of transcription is high, while a “weak” promoter means the rate of transcription is relatively low.

A promoter of the disclosure can be a Polymerase II (Pol II) promoter. Polymerase II transcribes all protein coding and many non-coding genes. A representative Pol II promoter includes a core promoter, which is a sequence of about 100 base pairs surrounding the transcription start site, and serves as a binding platform for the Pol II polymerase and associated general transcription factors. The promoter may contain one or more core promoter elements such as the TATA box, BRE, Initiator (INR), motif ten element (MTE), downstream core promoter element (DPE), downstream core element (DCE), although core promoters lacking these elements are known in the art.

A promoter of the disclosure can be a Polymerase III (Pol III) promoter. Pol III transcribes DNA to synthesize small ribosomal RNAs such as the 5S rRNA, tRNAs, and other small RNAs. Representative Pol III promoters use internal control sequences (sequences within the transcribed section of the gene) to support transcription, although upstream elements such as the TATA box are also sometimes used. All Pol III promoters are envisaged as within the scope of the instant disclosure.

The term “enhancer” refers to regulatory DNA sequences that, when bound by specific proteins called transcription factors, regulate the expression of an associated gene. Enhancers may be located in the intron of the gene, or 5′ or 3′ of the coding sequence of the gene. Enhancers may be proximal to the gene (i.e., within a few tens or hundreds of base pairs (bp) of the promoter), or may be located distal to the gene (i.e., thousands of bp, hundreds of thousands of bp, or even millions of bp away from the promoter). A single gene may be regulated by more than one enhancer, all of which are envisaged as within the scope of the instant disclosure.

As used herein, a “post-transcriptional regulatory element (PTRE, or TRE),” such as a hepatitis PTRE, refers to a DNA sequence that, when transcribed creates a tertiary structure capable of exhibiting post-transcriptional activity to enhance or promote expression of an associated gene operably linked thereto.

“Recombinant,” as used herein, means that a particular nucleic acid (DNA or RNA) is the product of various combinations of cloning, restriction, and/or ligation steps resulting in a construct having a structural coding or non-coding sequence distinguishable from endogenous nucleic acids found in natural systems. Generally, DNA sequences encoding the structural coding sequence can be assembled from cDNA fragments and short oligonucleotide linkers, or from a series of synthetic oligonucleotides, to provide a synthetic nucleic acid which is capable of being expressed from a recombinant transcriptional unit contained in a cell or in a cell-free transcription and translation system. Such sequences can be provided in the form of an open reading frame uninterrupted by internal non-translated sequences, or introns, which are typically present in eukaryotic genes. Genomic DNA comprising the relevant sequences can also be used in the formation of a recombinant gene or transcriptional unit. Sequences of non-translated DNA may be present 5′ or 3′ from the open reading frame, where such sequences do not interfere with manipulation or expression of the coding regions, and may indeed act to modulate production of a desired product by various mechanisms (see “enhancers” and “promoters”, above).

The term “recombinant polynucleotide” or “recombinant nucleic acid” refers to one which is not naturally occurring, e.g., is made by the artificial combination of two otherwise separated segments of sequence through human intervention. This artificial combination is often accomplished by either chemical synthesis means, or by the artificial manipulation of isolated segments of nucleic acids, e.g., by genetic engineering techniques. Such is usually done to replace a codon with a redundant codon encoding the same or a conservative amino acid, while typically introducing or removing a sequence recognition site. Alternatively, it is performed to join together nucleic acid segments of desired functions to generate a desired combination of functions. This artificial combination is often accomplished by either chemical synthesis means, or by the artificial manipulation of isolated segments of nucleic acids, e.g., by genetic engineering techniques.

Similarly, the term “recombinant polypeptide” or “recombinant protein” refers to a polypeptide or protein which is not naturally occurring, e.g., is made by the artificial combination of two otherwise separated segments of amino sequence through human intervention. Thus, e.g., a protein that comprises a heterologous amino acid sequence is recombinant.

As used herein, the term “contacting” means establishing a physical connection between two or more entities. For example, contacting a target nucleic acid with a guide nucleic acid means that the target nucleic acid and the guide nucleic acid are made to share a physical connection; e.g., can hybridize if the sequences share sequence similarity.

“Dissociation constant”, or “K_{d}”, are used interchangeably and mean the affinity between a ligand “L” and a protein “P”; i.e., how tightly a ligand binds to a particular protein. It can be calculated using the formula K_{d}=[L][P]/[LP], where [P], [L] and [LP] represent molar concentrations of the protein, ligand and complex, respectively.

The disclosure provides systems and methods useful for editing a target nucleic acid sequence. As used herein “editing” is used interchangeably with “modifying” and “modification” and includes but is not limited to cleaving, nicking, deleting, knocking in, knocking out, and the like.

By “cleavage” it is meant the breakage of the covalent backbone of a target nucleic acid molecule (e.g., RNA, DNA). Cleavage can be initiated by a variety of methods including, but not limited to, enzymatic or chemical hydrolysis of a phosphodiester bond. Both single-stranded cleavage and double-stranded cleavage are possible, and double-stranded cleavage can occur as a result of two distinct single-stranded cleavage events.

The term “knock-out” refers to the elimination of a gene or the expression of a gene. For example, a gene can be knocked out by either a deletion or an addition of a nucleotide sequence that leads to a disruption of the reading frame. As another example, a gene may be knocked out by replacing a part of the gene with an irrelevant sequence. The term “knock-down” as used herein refers to reduction in the expression of a gene or its gene product(s). As a result of a gene knock-down, the protein activity or function may be attenuated or the protein levels may be reduced or eliminated.

As used herein, “homology-directed repair” (HDR) refers to the form of DNA repair that takes place during repair of double-strand breaks in cells. This process requires nucleotide sequence homology, and uses a donor template to repair or knock-out a target DNA, and leads to the transfer of genetic information from the donor to the target. Homology-directed repair can result in an alteration of the sequence of the target sequence by insertion, deletion, or mutation if the donor template differs from the target DNA sequence and part or all of the sequence of the donor template is incorporated into the target DNA.

As used herein, “non-homologous end joining” (NHEJ) refers to the repair of double-strand breaks in DNA by direct ligation of the break ends to one another without the need for a homologous template (in contrast to homology-directed repair, which requires a homologous sequence to guide repair). NHEJ often results in the loss (deletion) of nucleotide sequence near the site of the double-strand break.

As used herein “micro-homology mediated end joining” (MMEJ) refers to a mutagenic DSB repair mechanism, which always associates with deletions flanking the break sites without the need for a homologous template (in contrast to homology-directed repair, which requires a homologous sequence to guide repair). MMEJ often results in the loss (deletion) of nucleotide sequence near the site of the double-strand break.

A polynucleotide or polypeptide has a certain percent “sequence similarity” or “sequence identity” to another polynucleotide or polypeptide, meaning that, when aligned, that percentage of bases or amino acids are the same, and in the same relative position, when comparing the two sequences. Sequence similarity (sometimes referred to as percent similarity, percent identity, or homology) can be determined in a number of different manners. To determine sequence similarity, sequences can be aligned using the methods and computer programs that are known in the art, including BLAST, available over the world wide web at ncbi.nlm.nih.gov/BLAST. Percent complementarity between particular stretches of nucleic acid sequences within nucleic acids can be determined using any convenient method. Example methods include BLAST programs (basic local alignment search tools) and PowerBLAST programs (Altschul et al., J. Mol. Biol., 1990, 215, 403-410; Zhang and Madden, Genome Res., 1997, 7, 649-656) or by using the Gap program (Wisconsin Sequence Analysis Package, Version 8 for Unix, Genetics Computer Group, University Research Park, Madison Wis.), e.g., using default settings, which uses the algorithm of Smith and Waterman (Adv. Appl. Math., 1981, 2, 482-489).

The terms “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. The term includes fusion proteins, including, but not limited to, fusion proteins with a heterologous amino acid sequence.

A “vector” or “expression vector” is a replicon, such as plasmid, phage, virus, or cosmid, to which another DNA segment, i.e., an expression cassette, may be attached so as to bring about the replication or expression of the attached segment in a cell.

The term “naturally-occurring” or “unmodified” or “wild type” as used herein as applied to a nucleic acid, a polypeptide, a cell, or an organism, refers to a nucleic acid, polypeptide, cell, or organism that is found in nature.

As used herein, a “mutation” refers to an insertion, deletion, substitution, duplication, or inversion of one or more amino acids or nucleotides as compared to a wild-type or reference amino acid sequence or to a wild-type or reference nucleotide sequence.

As used herein the term “isolated” is meant to describe a polynucleotide, a polypeptide, or a cell that is in an environment different from that in which the polynucleotide, the polypeptide, or the cell naturally occurs. An isolated genetically modified host cell may be present in a mixed population of genetically modified host cells.

A “host cell,” as used herein, denotes a eukaryotic cell, a prokaryotic cell, or a cell from a multicellular organism (e.g., a cell line) cultured as a unicellular entity, which eukaryotic or prokaryotic cells are used as recipients for a nucleic acid (e.g., an AAV vector), and include the progeny of the original cell which has been genetically modified by the nucleic acid. It is understood that the progeny of a single cell may not necessarily be completely identical in morphology or in genomic or total DNA complement as the original parent, due to natural, accidental, or deliberate mutation. A “recombinant host cell” (also referred to as a “genetically modified host cell”) is a host cell into which has been introduced a heterologous nucleic acid, e.g., an AAV vector.

The term “tropism” as used herein refers to preferential entry of the CasX delivery particle (referred to herein as XDP) into certain cell or tissue type(s) and/or preferential interaction with the cell surface that facilitates entry into certain cell or tissue types, optionally and preferably followed by expression (e.g., transcription and, optionally, translation) of sequences carried by the XDP into the cell.

The terms “pseudotype” or “pseudotyping” as used herein, refers to viral envelope proteins that have been substituted with those of another virus possessing preferable characteristics. For example, HIV can be pseudotyped with vesicular stomatitis virus G-protein (VSV-G) envelope proteins (amongst others, described herein, below), which allows HIV to infect a wider range of cells because HIV envelope proteins target the virus mainly to CD4+ presenting cells.

The term “tropism factor” as used herein refers to components integrated into the surface of an XDP that provides tropism for a certain cell or tissue type. Non-limiting examples of tropism factors include glycoproteins, antibody fragments (e.g., scFv, nanobodies, linear antibodies, etc.), receptors and ligands to target cell markers.

A “target cell marker” refers to a molecule expressed by a target cell including but not limited to cell-surface receptors, cytokine receptors, antigens, tumor-associated antigens, glycoproteins, oligonucleotides, enzymatic substrates, antigenic determinants, or binding sites that may be present in the on the surface of a target tissue or cell that may serve as ligands for an antibody fragment or glycoprotein tropism factor.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide-containing side chains consists of asparagine and glutamine; a group of amino acids having aromatic side chains consists of phenylalanine, tyrosine, and tryptophan; a group of amino acids having basic side chains consists of lysine, arginine, and histidine; and a group of amino acids having sulfur-containing side chains consists of cysteine and methionine. Exemplary conservative amino acid substitution groups are: valine-leucine-isoleucine, phenylalanine-tyrosine, lysine-arginine, alanine-valine, and asparagine-glutamine.

The term “antibody,” as used herein, encompasses various antibody structures, including but not limited to monoclonal antibodies, polyclonal antibodies, multispecific antibodies (e.g., bispecific antibodies), nanobodies, single domain antibodies such as VHH antibodies, and antibody fragments so long as they exhibit the desired antigen-binding activity or immunological activity. Antibodies represent a large family of molecules that include several types of molecules, such as IgD, IgG, IgA, IgM and IgE.

An “antibody fragment” refers to a molecule other than an intact antibody that comprises a portion of an intact antibody and that binds the antigen to which the intact antibody binds. Examples of antibody fragments include but are not limited to Fv, Fab, Fab′, Fab′-SH, F(ab′)2, diabodies, single chain diabodies, linear antibodies, a single domain antibody, a single domain camelid antibody, single-chain variable fragment (scFv) antibody molecules, and multispecific antibodies formed from antibody fragments.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” means a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, dogs, rabbits, mice, rats and other rodents.

As used herein, “treatment” or “treating,” are used interchangeably herein and refer to an approach for obtaining beneficial or desired results, including but not limited to a therapeutic benefit and/or a prophylactic benefit. By therapeutic benefit is meant eradication or amelioration of the underlying disorder or disease being treated. A therapeutic benefit can also be achieved with the eradication or amelioration of one or more of the symptoms or an improvement in one or more clinical parameters associated with the underlying disease such that an improvement is observed in the subject, notwithstanding that the subject may still be afflicted with the underlying disorder.

The terms “therapeutically effective amount” and “therapeutically effective dose”, as used herein, refer to an amount of a drug or a biologic, alone or as a part of a composition, that is capable of having any detectable, beneficial effect on any symptom, aspect, measured parameter or characteristics of a disease state or condition when administered in one or repeated doses to a subject such as a human or an experimental animal. Such effect need not be absolute to be beneficial.

As used herein, “administering” is meant a method of giving a dosage of a compound (e.g., a composition of the disclosure) or a composition (e.g., a pharmaceutical composition) to a subject.

A “subject” is a mammal. Mammals include, but are not limited to, domesticated animals, non-human primates, humans, rabbits, mice, rats and other rodents.

All publications, patents, and patent applications mentioned in this specification are herein incorporated by reference to the same extent as if each individual publication, patent, or patent application was specifically and individually indicated to be incorporated by reference.

### I. General Methods

The practice of the present invention employs, unless otherwise indicated, conventional techniques of immunology, biochemistry, chemistry, molecular biology, microbiology, cell biology, genomics and recombinant DNA, which can be found in such standard textbooks as Molecular Cloning: A Laboratory Manual, 3rd Ed. (Sambrook et al., Harbor Laboratory Press 2001); Short Protocols in Molecular Biology, 4th Ed. (Ausubel et al. eds., John Wiley & Sons 1999); Protein Methods (Bollag et al., John Wiley & Sons 1996); Nonviral Vectors for Gene Therapy (Wagner et al. eds., Academic Press 1999); Viral Vectors (Kaplift & Loewy eds., Academic Press 1995); Immunology Methods Manual (I. Lefkovits ed., Academic Press 1997); and Cell and Tissue Culture: Laboratory Procedures in Biotechnology (Doyle & Griffiths, John Wiley & Sons 1998), the disclosures of which are incorporated herein by reference.

Where a range of values is provided, it is understood that endpoints are included and that each intervening value, to the tenth of the unit of the lower limit unless the context clearly dictates otherwise, between the upper and lower limit of that range and any other stated or intervening value in that stated range, is encompassed. The upper and lower limits of these smaller ranges may independently be included in the smaller ranges, and are also encompassed, subject to any specifically excluded limit in the stated range. Where the stated range includes one or both of the limits, ranges excluding either or both of those included limits are also included.

Unless defined otherwise, all technical and scientific terms used herein have the same meaning as commonly understood by one of ordinary skill in the art to which this invention belongs. All publications mentioned herein are incorporated herein by reference to disclose and describe the methods and/or materials in connection with which the publications are cited.

It must be noted that as used herein and in the appended claims, the singular forms “a,” “an,” and “the” include plural referents unless the context clearly dictates otherwise.

It will be appreciated that certain features of the disclosure, which are, for clarity, described in the context of separate embodiments, may also be provided in combination in a single embodiment. In other cases, various features of the disclosure, which are, for brevity, described in the context of a single embodiment, may also be provided separately or in any suitable sub-combination. It is intended that all combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

### II. Systems for Genetic Editing and Gene-Editing Pairs

In a first aspect, the present disclosure provides systems comprising engineered CasX nuclease proteins and engineered guide ribonucleic acid scaffolds (ERS) for use in modifying or editing a target nucleic acid of a gene, inclusive of coding and non-coding regions (a eCasX:ERS system). Generally, any portion of a gene can be targeted using the programable systems and methods provided herein.

As used herein, a “system”, used interchangeably with “composition”, can comprise an engineered CasX nuclease protein and one or more ERS (with linked targeting sequences) of the disclosure as gene editing pairs, nucleic acids encoding the engineered CasX nuclease proteins and ERS, as well as vectors or particle delivery formulations comprising the nucleic acids or engineered CasX proteins and ERS of the disclosure.

In some embodiments, the disclosure provides systems specifically designed to modify the target nucleic acid of a gene in eukaryotic cells; either in vitro, ex vivo, or in vivo in a subject. The engineered CasX of the disclosure are Class 2, Type V CRISPR nucleases. Although members of Class 2 Type V CRISPR-Cas nucleases have differences, they share some common characteristics that distinguish them from the Cas9 systems. Firstly, the Type V nucleases possess an RNA-guided single effector containing a RuvC domain but no HNH domain, and they recognize a TC motif PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the disclosure provides engineered CasX proteins designed with multiple mutations relative to a CasX from which it was derived, wherein the engineered CasX has improved properties, while retaining the ability to complex with a guide ribonucleic acid and retaining nuclease activity.

Provided herein are systems comprising an engineered CasX protein and an engineered guide ribonucleic acid scaffold (ERS) that, together with a targeting sequence linked to the 3′ end of the scaffold are referred to herein as a gene editing pair. An ERS and an engineered CasX protein can bind together via non-covalent interactions to form a gene editing pair complex, referred to herein as a ribonucleoprotein (RNP) complex (it being understood that, in all cases for use in editing a target nucleic acid, the ERS would have a linked targeting sequence). In some embodiments, the use of a pre-complexed RNP of an engineered CasX and ERS confers advantages in the delivery of the system components to a cell or target nucleic acid for editing of the target nucleic acid. In the RNP, the ERS can provide target specificity to the RNP complex by including a targeting sequence (or “spacer”) having a nucleotide sequence that is complementary to and capable of binding to a sequence of a target nucleic acid. In the RNP, the engineered CasX protein of the pre-complexed RNP provides the site-specific activity and is guided to a target site (and further stabilized at a target site) within a target nucleic acid sequence to be modified by virtue of its association with the ERS. The engineered CasX protein of the RNP complex provides the site-specific activities of the complex such as binding, cleavage, or nicking of the target nucleic acid sequence by the engineered CasX protein. Provided herein are systems and cells comprising the engineered CasX proteins, ERS, and gene editing pairs of any combination of the engineered CasX and ERS embodiments described herein, as well as delivery modalities comprising or encoding the engineered CasX and ERS. Each of these components and their use in the editing of the target nucleic acid of a gene is described herein, below.

In some embodiments, the disclosure provides systems of gene editing pairs comprising an engineered CasX protein selected from any one of the engineered CasX proteins selected from the group consisting of SEQ ID NOS: 247-294, 24916-49628, 49746-49747, and 49871-49873, or a sequence having at least about 85%, at least about 90%, or at least about 95%, or at least about 95%, or at least about 96%, or at least about 97%, or at least about 98%, or at least about 99% sequence identity thereto and an ERS selected from the group consisting of SEQ ID NOS: 156, 739-907, 11568-22227, 23572-24915, and 49719-49735, or sequence variants having at least 60%, or at least 70%, at least about 80%, or at least about 90%, or at least about 95%, or at least about 96%, or at least about 97%, or at least about 98%, or at least about 99% sequence identity thereto, wherein the ERS comprises a targeting sequence complementary to the target nucleic acid. In some embodiments, the guide ribonucleic acid is an ERS selected from the group consisting of SEQ ID NOS: 156, 739-907, 11568-22227, 23572-24915, and 49719-49735, wherein the ERS comprises a targeting sequence complementary to the target nucleic acid, or a sequence with at least at least 1, 2, 3, 4, or 5 mismatches thereto.

In some embodiments, the disclosure provides systems of gene editing pairs comprising an engineered CasX protein selected from the group consisting of SEQ ID NOS: 24916-49628, 49746-49747, and 49871-49873, or a sequence having at least about 85%, at least about 90%, or at least about 95%, or at least about 95%, or at least about 96%, or at least about 97%, or at least about 98%, or at least about 99% sequence identity thereto wherein the engineered CasX comprises a sequence having one or more mutations relative to the sequence of SEQ ID NO: 228, wherein the mutations result in an improved characteristic compared to unmodified SEQ ID NO: 228. In some embodiments, the disclosure provides systems of gene editing pairs comprising an engineered CasX protein selected from the group consisting of SEQ ID NOS: 49746-49747, and 49871-49873, or a sequence having at least about 85%, at least about 90%, or at least about 95%, or at least about 95%, or at least about 96%, or at least about 97%, or at least about 98%, or at least about 99% sequence identity thereto wherein the engineered CasX comprises a sequence having one or more mutations relative to the sequence of SEQ ID NO: 228, wherein the mutations result in an improved characteristic compared to unmodified SEQ ID NO: 228, wherein the improved characteristic is one or more of improved editing activity of the target nucleic acid, improved editing specificity for the target nucleic acid, improved editing specificity ratio for the target nucleic acid, decreased off-target editing, increased percentage of a eukaryotic genome that can be efficiently edited, improved ability to form cleavage-competent RNP with an ERS, and improved stability of an RNP complex. In some embodiments, the ERS of the gene editing pair is selected from the group consisting of SEQ ID NOS: 156, 739-907, 11568-22227, 23572-24915, and 49719-49735, or sequence variants having at least 60%, or at least 70%, at least about 80%, or at least about 90%, or at least about 95%, or at least about 96%, or at least about 97%, or at least about 98%, or at least about 99% sequence identity thereto, and wherein the ERS comprises a targeting sequence complementary to the target nucleic acid. In some embodiments, the guide ribonucleic acid is an ERS selected from the group consisting of SEQ ID NOS: 156, 739-907, 11568-22227, 23572-24915, and 49719-49735, wherein the ERS comprises a targeting sequence complementary to the target nucleic acid, or a sequence with at least at least 1, 2, 3, 4, or 5 mismatches thereto. In some embodiments, the disclosure provides systems of gene editing pairs comprising an engineered CasX protein comprising a pair of mutations as depicted in Table 22, or further variations thereof. In some embodiments, the disclosure provides systems of gene editing pairs comprising an engineered CasX protein comprising a pair of mutations as depicted in Table 22, or sequence variants having at least 60%, or at least 70%, at least about 80%, or at least about 90%, or at least about 95%, or at least about 96%, or at least about 97%, or at least about 98%, or at least about 99% sequence identity thereto and an ERS comprising one or more mutations of Table 44, Table 45 and Table 47 or an ERS selected from the group consisting of SEQ ID NOS: 156, 739-907, 11568-22227, 23572-24915, and 49719-49735, or sequence variants having at least 60%, or at least 70%, at least about 80%, or at least about 90%, or at least about 95%, or at least about 96%, or at least about 97%, or at least about 98%, or at least about 99% sequence identity thereto. In some embodiments of the system, the RNP of the gene editing pair is capable of binding and cleaving the double strand of a target nucleic acid, including a coding sequence, a complement of a coding sequence, a non-coding sequence, and to regulatory elements. In some embodiments of the system, the RNP of the gene editing pair is capable of binding a target nucleic acid and generating one or more single-stranded nicks in the target nucleic acid.

In other embodiments, the disclosure provides systems of a gene editing pair comprising the engineered CasX protein, a first ERS with a targeting sequence as described herein, and a second ERS, wherein the second ERS has a targeting sequence complementary to a different or overlapping portion of the target nucleic acid compared to the targeting sequence of the first ERS, introducing multiple breaks in the target nucleic acid that result in permanent indels or mutations in the target nucleic acid, or an excision of the intervening sequence between the breaks.

In some embodiments, the gene editing pair of an engineered CasX and an ERS has one or more improved characteristics compared to a gene editing pair comprising a CasX variant from which the engineered CasX was derived (e.g., CasX 515, SEQ ID NO: 228) and the gRNA variant from which the ERS was derived (e.g., gRNA scaffolds 174, 175, 221 or 235. In the foregoing embodiment, the one or more improved characteristics can be assayed in an in vitro assay under comparable conditions for the gene editing pair and the CasX variant and gRNA variant from which it was derived, or in vivo in a subject. Exemplary improved characteristics, as described herein, may, in some embodiments, include increased RNP complex stability, increased binding affinity between the engineered CasX and ERS, improved kinetics of RNP complex formation, higher percentage of cleavage-competent RNP, increased editing activity for the target nucleic acid, increased editing specificity, decreased off-target editing, and enhanced utilization of non-canonical PAM sequences.

In some embodiments, the disclosure provides compositions of gene editing pairs of any of the embodiments disclosed herein for use in the manufacture of a medicament for the treatment of a subject having a disease.

In other embodiments, the disclosure provides vectors encoding or comprising the engineered CasX and/or ERS for the production and/or delivery of the systems. Also provided herein are methods of making engineered CasX proteins and ERS, as well as methods of using the engineered CasX and ERS, including methods of gene editing and methods of treatment. The engineered CasX proteins and ERS components of the systems and their features, as well as delivery modalities and the methods of using the systems are described more fully, below.

### III. Engineered Ribonucleic Acid Scaffolds (ERS) and Targeting Sequences of the Systems for Genetic Editing

In another aspect, the disclosure relates to engineered guide ribonucleic acid scaffolds (ERS) that, when linked with targeting sequences complementary to (and are therefore able to hybridize with) a target nucleic acid sequence of a gene, have utility, when complexed with an engineered CasX nuclease protein, in genome editing of a target nucleic acid in vitro, ex vivo, or in vivo in a subject. The ERS of the disclosure are guide ribonucleic acid scaffolds that are modified relative to reference gRNA and gRNA variants by approaches described herein.

Collectively, the CasX guide ribonucleic acids of the disclosure, including all ERS of the embodiments, reference gRNA and gRNA variants, comprise distinct structured regions, or domains; the RNA triplex, the scaffold stem loop, the extended stem loop, the pseudoknot, and the targeting sequence that, in the embodiments of the disclosure is specific for a target nucleic acid and is located on the 3′end of the guide scaffold. The 5′ end, RNA triplex, the scaffold stem loop, the pseudoknot and the extended stem loop, together with the unstructured triplex loop that bridges portions of the triplex, together, are referred to as the “scaffold” of the guide RNA and ERS. In some cases, the scaffold stem further comprises a bubble. In other cases, the scaffold further comprises a triplex loop region. In still other cases, the scaffold further comprises a 5′ unstructured region. In some embodiments, the ERS of the disclosure for use in the systems comprise a scaffold stem loop having the sequence of CCAGCGACUAUGUCGUAGUGG (SEQ ID NO: 49737).

The properties and characteristics of CasX guide ribonucleic acids and their domains are described in WO2020247882A1, US20220220508A1, and WO2022120095A1, incorporated by reference herein. Each of the structured domains contribute to establishing the global RNA fold of the guide and retain functionality of the guide; particularly the ability to properly complex with the CasX nuclease. For example, the guide scaffold stem interacts with the helical I domain of CasX nuclease, while residues within the triplex, triplex loop, and pseudoknot stem interact with the OBD of the CasX nuclease. Together, these interactions confer the ability of the guide to bind and form an RNP with the CasX that retains stability, while the spacer (or targeting sequence) directs and defines the specificity of the RNP for binding a specific sequence of DNA. The individual domains are described more fully, below.

In the embodiments, the ERS are single guide constructs, rather than the double stranded duplex of wild-type guides, wherein the “activator” and the “targeter” are covalently linked together by intervening nucleotides.

The targeting sequence linked to the 3′ end of an ERS includes a nucleotide sequence (referred to interchangeably as a guide sequence, a spacer, a targeter, or a targeting sequence) that is complementary to (and therefore hybridizes with) a specific sequence (a target site) within the target nucleic acid sequence (e.g., a strand of a double stranded target DNA, a target ssRNA, a target ssDNA, etc.), described more fully below. The targeting sequence linked to an ERS is capable of binding to a target nucleic acid sequence, including, in the context of the present disclosure, a coding sequence, a complement of a coding sequence, a non-coding sequence, and to accessory elements. The protein-binding segment (or “activator” or “protein-binding sequence”) interacts with (e.g., binds to) a CasX protein as a complex, forming an RNP (described more fully, below).

Site-specific binding and/or cleavage of a target nucleic acid sequence (e.g., genomic DNA) by the engineered CasX protein can occur at one or more locations (e.g., a sequence of a target nucleic acid) determined by base-pairing complementarity between the targeting sequence of the ERS and the target nucleic acid sequence. Thus, for example, the ERS of the disclosure with a linked targeting sequence have sequences complementarity to and therefore can hybridize with the target nucleic acid that is adjacent to a sequence complementary to a TC PAM motif or a PAM sequence, such as ATC, CTC, GTC, or TTC. Because the targeting sequence of a guide sequence hybridizes with a sequence of a target nucleic acid sequence, a targeter can be modified by a user to hybridize with a specific target nucleic acid sequence, so long as the location of the PAM sequence is considered. By selection of the targeting sequences of the ERS, defined regions of the target nucleic acid sequence or sequences bracketing a particular location within the target nucleic acid can be modified or edited using the systems described herein. In some embodiments, the targeting sequence of the ERS has between 15 and 20 consecutive nucleotides. In some embodiments, the targeting sequence has 15, 16, 17, 18, 19, or 20 consecutive nucleotides. In some embodiments, the targeting sequence consists of 20 consecutive nucleotides. In some embodiments, the targeting sequence consists of 19 consecutive nucleotides. In some embodiments, the targeting sequence consists of 18 consecutive nucleotides. In some embodiments, the targeting sequence consists of 17 consecutive nucleotides. In some embodiments, the targeting sequence consists of 16 consecutive nucleotides. In some embodiments, the targeting sequence consists of 15 consecutive nucleotides. In some cases, an ERS targeting sequence linked to an ERS scaffold of the disclosure is complementary to and hybridizes with a gene exon. In some embodiments, an ERS targeting sequence is complementary to and hybridizes with a sequence of a splice-acceptor site of an exon. In other embodiments, an ERS targeting sequence hybridizes with an intron. In other embodiments, an ERS targeting sequence hybridizes with an intron-exon junction. In other embodiments, an ERS targeting sequence hybridizes with an intergenic region of the gene. In other embodiments, an ERS targeting sequence hybridizes with a regulatory region. In some cases, the regulatory region is a promoter or enhancer. In some cases, the regulatory region is located 5′ of the transcription start site or 3′ of the transcription start. In some cases, the regulatory region is in an intron of the gene. In other cases, the regulatory region comprises the 5′ UTR of the gene. In still other cases, the regulatory region comprises the 3′UTR of the gene.

By selection of the targeting sequences of the gRNA, defined regions of the target nucleic acid sequence can be modified or edited using the CasX:gRNA systems described herein. In some embodiments, the gRNA and linked targeting sequence exhibit a low degree of off-target effects to the DNA of a cell. As used herein, “off-target effects” refers to effects of unintended cleavage, such as mutations and indel formation, at untargeted genomic sites showing a similar but not an identical sequence compared to the target site (i.e., the targeting sequence of the gRNA). In some embodiments, the off-target effects exhibited by the gRNA and linked targeting sequence are less than about 5%, less than about 4%, less than 3%, less than about 2%, less than about 1%, less than about 0.5%, less than 0.1% in cells. In some embodiments, the off-target effects are determined in silico. In some embodiments, the off-target effects are determined in an in vitro cell-free assay. In some embodiments, the off-target effects are determined in a cell-based assay.

In some embodiments, the systems of the disclosure comprises a first ERS and further comprises a second (and optionally a third, fourth, fifth, or more) ERS, wherein the second ERS or additional ERS has a targeting sequence complementary to a different or overlapping portion of the target nucleic acid sequence compared to the targeting sequence of the first ERS such that multiple points in the target nucleic acid are targeted, and, for example, multiple breaks are introduced in the target nucleic acid by the engineered CasX, which is then edited by non-homologous end joining (NHEJ), homology-directed repair (HDR), homology-independent targeted integration (HITI), micro-homology mediated end joining (MMEJ), single strand annealing (SSA) or base excision repair (BER). It will be understood that in such cases, the second or additional ERS is complexed with an additional copy of the engineered CasX protein. By selection of the targeting sequences linked to the ERS, defined regions of the target nucleic acid sequence bracketing a particular location within the target nucleic acid can be modified or edited using the systems described herein, including facilitating the insertion of a donor template or excision of a region or exon comprising a mutation of the targeted gene by a double-cut mechanism with paired engineered CasX and ERS having different targeting sequences such that the intervening nucleotides are excised.

a. Reference gRNA

As used herein, a “reference gRNA” refers to a CRISPR guide ribonucleic acid comprising a wild-type sequence of a naturally-occurring gRNA. In some embodiments, a CasX reference gRNA comprises a sequence isolated or derived from Deltaproteobacter. In some embodiments, a CasX reference guide RNA comprises a sequence isolated or derived from Planctomycetes. In still other embodiments, a CasX reference gRNA comprises a sequence isolated or derived from Candidatus Sungbacteria.

Table 1 provides the sequences of reference gRNAs tracr and scaffold sequences. In some embodiments, the disclosure provides ERS sequences wherein the gRNA has a scaffold comprising a sequence having one or more nucleotide modifications relative to a reference gRNA sequence having a sequence of any one of SEQ ID NOS: 4-16 of Table 1.

TABLE 1
Reference gRNA tracr and scaffold sequences

| SEQ ID NO | Nucleotide Sequence |
|---|---|
| 4 | ACAUCUGGCGCGUUUAUUCCAUUACUUUGGAGCCAGUCCCAGCGACUAUGUCGUAUGGACGAAG |
| | CGCUUAUUUAUCGGAGAGAAACCGAUAAGUAAAACGCAUCAAAG |
| 5 | UACUGGCGCUUUUAUCUCAUUACUUUGAGAGCCAUCACCAGCGACUAUGUCGUAUGGGUAAAGC |
| | GCUUAUUUAUCGGAGAGAAAUCCGAUAAAUAAGAAGCAUCAAAG |
| 6 | ACAUCUGGCGCGUUUAUUCCAUUACUUUGGAGCCAGUCCCAGCGACUAUGUCGUAUGGACGAAG |
| | CGCUUAUUUAUCGGAGA |
| 7 | ACAUCUGGCGCGUUUAUUCCAUUACUUUGGAGCCAGUCCCAGCGACUAUGUCGUAUGGACGAAG |
| | CGCUUAUUUAUCGG |
| 8 | UACUGGCGCUUUUAUCUCAUUACUUUGAGAGCCAUCACCAGCGACUAUGUCGUAUGGGUAAAGC |
| | GCUUAUUUAUCGGAGA |
| 9 | UACUGGCGCUUUUAUCUCAUUACUUUGAGAGCCAUCACCAGCGACUAUGUCGUAUGGGUAAAGC |
| | GCUUAUUUAUCGG |
| 10 | GUUUACACACUCCCUCUCAUAGGGU |
| 11 | GUUUACACACUCCCUCUCAUGAGGU |
| 12 | UUUUACAUACCCCCUCUCAUGGGAU |
| 13 | GUUUACACACUCCCUCUCAUGGGGG |
| 14 | CCAGCGACUAUGUCGUAUGG |
| 15 | GCGCUUAUUUAUCGGAGAGAAAUCCGAUAAAUAAGAAGC |
| 16 | GGCGCUUUUAUCUCAUUACUUUGAGAGCCAUCACCAGCGACUAUGUCGUAUGGGUAAAGCGCUU |
| | AUUUAUCGGA |

b. Engineered Ribonucleic Acid Scaffolds (ERS)

In another aspect, the disclosure relates to ERS for use in the systems of the disclosure that comprise multiple modifications relative to a gRNA variant scaffold of Table 2 from which it was derived. All ERS that have one or more improved functions, characteristics, or add one or more new functions when the ERS is compared to a gRNA variant from which it was derived, while retaining the functional properties of being able to complex with the engineered CasX as an RNP and guide the engineered CasX ribonucleoprotein holo complex to the target nucleic acid are envisaged as within the scope of the disclosure. It will be understood that although the present disclosure is focused on ERS and engineered CasX, an ERS also retain the ability to complex with reference CasX and CasX variants to form an RNP and an engineered CasX retains the ability to complex with reference gRNA and gRNA variants to form an RNP In some embodiments, the ERS has an improved characteristic selected from the group consisting of enhanced folding stability of individual regions within the scaffold, enhanced folding stability of the entire scaffold, enhanced transcriptional efficiency, enhanced binding affinity to the engineered CasX nuclease, increased editing when complexed as an RNP, increased cleavage activity when complexed as an RNP, and increased specificity of the RNP in complex with a target nucleic acid. In some cases of the foregoing, the improved characteristic can be assessed in an in vitro assay, including the assays of the Examples. In other cases of the foregoing, the improved characteristic is assessed in vivo. In some cases, the one or more of the improved characteristics of the ERS is relative to the reference gRNA of SEQ ID NO: 4 or SEQ ID NO: 5, or to gRNA variant 174, 175, 221, or 235 (SEQ ID NOS: 17, 18, 61, and 75, respectively).

In some embodiments, a new ERS can be created by subjecting a gRNA variant to one or more mutagenesis methods, such as the mutagenesis methods described herein in the Examples (e.g., Example 11, as well as in PCT/US2021/061673 and WO2020247882A1, incorporated by reference herein), which may include Deep Mutational Evolution (DME), deep mutational scanning (DMS), error prone PCR, cassette mutagenesis, random mutagenesis, staggered extension PCR, gene shuffling, substitution of a domain from one gRNA variant to another, or chemical modification in order to generate one or more ERS with enhanced or varied properties relative to the gRNA variant that was modified. The activity of the gRNA variant from which an ERS was derived may be used as a benchmark against which the activity of ERS is compared, thereby measuring improvements in function or other characteristics of the ERS. In other embodiments, a gRNA variant may be subjected to one or more deliberate, specifically-targeted mutations in order to produce an ERS; for example a rationally designed variant such as described herein in the Examples.

Table 2 provides exemplary gRNA variant scaffold sequences that, in some cases, provided the starting sequence from which the ERS were derived. In a particular embodiment, the gRNA variants 174, 175, 221, and 235 were subjected to mutagenesis to result in the ERS of SEQ ID NOS: 156, 739-907, 11568-22227, 23572-24915, and 49719-49735.

TABLE 2
Exemplary gRNA Variant Scaffold Sequences

| | Scaffold | |
|---|---|---|
| SEQ ID | variant | |
| NO | ID | Nucleotide sequence |
| 17 | 174 | ACUGGCGCUUUUAUCUGAUUACUUUGAGAGCCAUCACCAGCGACUAUGUCGUAGUG |
| | | GGUAAAGCUCCCUCUUCGGAGGGAGCAUCAAAG |
| 18 | 175 | ACUGGCGCCUUUAUCUCAUUACUUUGAGAGCCAUCACCAGCGACUAUGUCGUAUGG |
| | | GUAAAGCGCUUACGGACUUCGGUCCGUAAGAAGCAUCAAAG |
| 61 | 221 | ACUGGCACUUCUAUCUGAUUACUCUGAGAGCCAUCACCAGCGACUAUGUCGUAUGG |
| | | GUAAAGCCGCUUACGGACUUCGGUCCGUAAGAGGCAUCAGAG |
| 75 | 235 | ACUGGCGCUUCUAUCUGAUUACUCUGAGCGCCAUCACCAGCGACUAUGUCGUAGUG |
| | | GGUAAAGCCGCUUACGGACUUCGGUCCGUAAGAGGCAUCAGAG |

In some embodiments, an ERS of the disclosure comprises multiple modifications to the sequence of a previously generated gRNA variant, the previously generated variant itself serving as the sequence to be modified. In some cases, one or modifications are introduced to one or more regions of the scaffold wherein the regions are selected from the group consisting of 5′ end, pseudoknot stem I, triplex loop (including triplex regions I and II), pseudoknot stem II, scaffold stem loop, extended stem loop, and triplex region III. In some embodiments, one or more modifications are introduced into the 5′ end of the scaffold. In some embodiments, one or more modifications are introduced into the pseudoknot region of the scaffold. In some embodiments, one or more modifications are introduced into triplex loop region of the scaffold. In some embodiments, one or more modifications are introduced into scaffold stem loop region of the scaffold. In some embodiments, one or more modifications are introduced into the extended stem loop region of the scaffold. In other cases, one or modifications are introduced to the scaffold bubble. In still other cases, one or more modifications are introduced into two or more of the foregoing regions. Such modifications can comprise an insertion, deletion, or substitution of one or more consecutive nucleotides; i.e., 1, 2, 1 to 5, 1 to 10, 1 to 20, or 1 to 30 or more consecutive nucleotides in the foregoing regions, or any combination thereof. In turn, the modifications to the foregoing regions can be combined to engineer an ERS with multiple modifications. Exemplary methods to generate and assess the modifications are described in Examples 8-12, and representative modifications and resulting sequences are presented in Tables 29, 30, 37, 38, 40, 43, 44, 45, 46, 47, 50

In some embodiments, the ERS comprises a sequence having at least about 70% sequence identity to (i) ACUGGCACUUCUAUCUGAUUACUCUGAGAGCCAUCACCAGCGACUAUGUCGUAU GGGUAAAGCCGCUUACGGACUUCGGUCCGUAAGAGGCAUCAGAG (SEQ ID NO: 61); or (ii) ACUGGCGCUUCUAUCUGAUUACUCUGAGCGCCAUCACCAGCGACUAUGUCGUAGU GGGUAAAGCUCCCUCUUCGGAGGGAGCAUCAGAG (SEQ ID NO: 156); comprising one or more modifications in the sequence, wherein the one or more modifications result in an improved characteristic compared to unmodified SEQ ID NO: 61 or SEQ ID NO: 156. In some embodiments, the ERS comprises at least two modifications in the sequences of SEQ ID NO: 61 or SEQ ID NO: 156, wherein the modifications result in an improved characteristic compared to unmodified SEQ ID NO: 61 or SEQ ID NO: 156. In some embodiments, the modification(s) comprise: i) a substitution of 1 to 30 consecutive nucleotides in one or more regions of the scaffold; ii) a deletion of 1 to 10 consecutive nucleotides in one or more regions of the scaffold; iii) an insertion of 1 to 10 consecutive nucleotides in one or more regions of the scaffold; iv) a substitution of the scaffold stem loop from a heterologous RNA source; v) a substitution of the extended stem loop with an RNA stem loop sequence from a heterologous RNA source; or vi) any combination of (i)-(v). In some embodiments, the modifications comprise mutations in one or more regions selected from the group consisting of a 5′ end, a pseudoknot stem, a triplex loop, a scaffold stem loop, an extended stem loop, and a triplex region III. In some embodiments, the modifications comprise mutations in at least two regions of the ERS, wherein the regions are selected from the group consisting of a 5′ end, a pseudoknot stem I, a triplex loop, a pseudoknot stem II, a scaffold stem loop, an extended stem loop, and a triplex region III. In some embodiments, the mutations are selected from the group consisting of the mutations set forth in any one of Tables 44, 45, or 47. In some embodiments, the ERS comprises individual mutated regions selected from the sequences of SEQ ID NOS: 739-753 in the 5′ end region, SEQ ID NOS: 754-772 in the triplex loop region, SEQ ID NOS: 773-791 in the triplex region, SEQ ID NOS: 792-841 in the pseudoknot region, SEQ ID NOS: 842-869 in the scaffold stem region, or SEQ ID NOS: 870-907 in the extended stem region. In some embodiments, the ERS comprises paired combinations of individual mutated sequences from different regions. In some embodiments, the ERS comprises a sequence selected from the group consisting of SEQ ID NOS: 11,568-22,227 and 23,572-24,915, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% sequence identity thereto.

In some embodiments, the disclosure provides ERS wherein the scaffold has about 85-100 nucleotides, or any integer in between. In some embodiments, the disclosure provides ERS wherein the scaffold has about 85-95 nucleotides, or about 88-90 nucleotides, or about 89 nucleotides,

In some embodiments, the disclosure provides an ERS comprising a sequence selected from the group consisting of SEQ ID NOS: 156, 739-907, 739-907, 11568-22227, 23572-24915, and 49719-49735, or a sequence having at least about 70%, at least about 80%, at least about 90%, at least about 91%, at least about 92%, at least about 93%, at least about 94%, at least about 95%, at least about 96%, at least about 97%, at least about 98%, or at least about 99% sequence identity thereto, wherein the ERS comprises an improved characteristic compared to the sequence of SEQ ID NO: 17, when assayed in an in vitro cell-based assay under comparable conditions. In the foregoing, the improved characteristic is one or more functional properties selected from the group consisting of improved binding to a CasX nuclease to form a ribonucleoprotein (RNP), improved folding stability of the ERS, increased half-life in a cell, increased transcriptional efficiency, enhanced ability to synthetically manufacture the ERS, improved editing activity of a target nucleic acid by an RNP comprising the ERS, and improved editing specificity by an RNP comprising the ERS.

In some embodiments, the ERS comprises an exogenous extended stem loop that has little or no identity to the reference stem loop regions disclosed herein (e.g., SEQ ID NO:15). In some embodiments, the heterologous stem loop increases the stability of the ERS. In some embodiments, the heterologous RNA stem loop is capable of binding a protein, an RNA structure, a DNA sequence, or a small molecule. In some embodiments, an exogenous stem loop region replacing the stem loop comprises an RNA stem loop or hairpin in which the resulting ERS has increased stability and, depending on the choice of loop, confers non-covalent recruitment with certain cellular proteins or RNA. Non-limiting examples of such non-covalent recruitment components include hairpin RNA or loops such as MS2 hairpin, PP7 hairpin, Qβ hairpin, boxB, transactivation response element (TAR), phage GA hairpin, phage ΛN hairpin, iron response element (IRE), and U1 hairpin II that have binding affinity for the NCR MS2 coat protein, PP7 coat protein, Qβ coat protein, protein N, protein Tat, phage GA coat protein, iron-responsive binding element (IRE) protein, and U1A signal recognition particle, respectively, that are incorporated in the protein-encoding nucleic acids used to transfect the packaging host cell. Such exogenous extended stem loops can comprise, for example a thermostable RNA such as MS2 hairpin (ACAUGAGGAUCACCCAUGU (SEQ ID NO: 215)), Qβ hairpin (UGCAUGUCUAAGACAGCA (SEQ ID NO: 216)), U1 hairpin II (AAUCCAUUGCACUCCGGAUU (SEQ ID NO: 217)), Uvsx (CCUCUUCGGAGG (SEQ ID NO: 218)), PP7 hairpin (AGGAGUUUCUAUGGAAACCCU (SEQ ID NO: 219)), Phage replication loop (AGGUGGGACGACCUCUCGGUCGUCCUAUCU (SEQ ID NO: 220)), Kissing loop_a (UGCUCGCUCCGUUCGAGCA (SEQ ID NO: 221)), Kissing loop_b1 (UGCUCGACGCGUCCUCGAGCA (SEQ ID NO: 222)), Kissing loop_b2 (UGCUCGUUUGCGGCUACGAGCA (SEQ ID NO: 223)), G quadriplex M3q (AGGGAGGGAGGGAGAGG (SEQ ID NO: 224)), G quadriplex telomere basket (GGUUAGGGUUAGGGUUAGG (SEQ ID NO: 225)), Sarcin-ricin loop (CUGCUCAGUACGAGAGGAACCGCAG (SEQ ID NO: 226)) or Pseudoknots (UACACUGGGAUCGCUGAAUUAGAGAUCGGCGUCCUUUCAUUCUAUAUACUUUGG AGUUUUAAAAUGUCUCUAAGUACA (SEQ ID NO: 227)). In some embodiments, one of the foregoing hairpin sequences is incorporated into the stem loop to help traffic the incorporation of the ERS (and an associated CasX in an RNP complex) into a budding XDP in a packaging host cell (described more fully, below) when the counterpart ligand is incorporated into the Gag polyprotein of the XDP.

c. Guide 316

Guide scaffolds can be made by several methods, including recombinantly or by solid-phase RNA synthesis. However, the length of the scaffold can affect the manufacturability when using solid-phase RNA synthesis, with longer lengths resulting in increased manufacturing costs, decreased purity and yield, and higher rates of synthesis failures. For use in lipid nanoparticle (LNP) formulations, solid-phase RNA synthesis of the scaffold is preferred in order to generate the quantities needed for commercial development. While previous experiments had identified gRNA variant 235 (SEQ ID NO: 75) as having enhanced properties relative to gRNA variants 174 (SEQ ID NO: 17), its increased length rendered its use for LNP formulations problematic. Accordingly, alternative sequences were sought. In some embodiments, the disclosure provides an ERS wherein the ERS scaffold and linked targeting sequence has a sequence less than about 115 nucleotides, less than about 110 nucleotides, or less than about 100 nucleotides. In some embodiments, the disclosure provides an ERS wherein the ERS scaffold and linked targeting sequence has a sequence between 100-115 nucleotides, or any integer in between.

In some embodiments, an ERS was designed wherein the scaffold 174 (SEQ ID NO: 17) sequence, was modified by introducing one, two, three, four or more mutations at positions selected from the group consisting of U11, U24, A29, and A87. In some embodiments, the ERS comprises a sequence of SEQ ID NO: 17, or a sequence having at least about 70% sequence identity thereto, comprising an extended stem loop sequence of SEQ ID NO: 49739 and one or more mutations at positions selected from the group consisting of U11, U24, A29, and A87. In some embodiments, the ERS comprises a sequence of SEQ ID NO: 17, or a sequence having at least about 70% sequence identity thereto, comprising an extended stem loop sequence of SEQ ID NO: 49739 and two mutations at positions selected from the group consisting of U11, U24, A29, and A87. In some embodiments, the ERS comprises a sequence of SEQ ID NO: 17, or a sequence having at least about 70% sequence identity thereto, comprising an extended stem loop sequence of SEQ ID NO: 49739 and three mutations at positions selected from the group consisting of U11, U24, A29, and A87. In some embodiments, the ERS comprises a sequence of SEQ ID NO: 17, or a sequence having at least about 70% sequence identity thereto, comprising an extended stem loop sequence of SEQ ID NO: 49739 and four mutations at positions selected from the group consisting of U11, U24, A29, and A87. In one embodiment of the foregoing, the mutations consist of U11C, U24C, A29C, and A87G, resulting in the ERS 316 sequence ACUGGCGCUUCUAUCUGAUUACUCUGAGCGCCAUCACCAGCGACUAUGUCGUAGU GGGUAAAGCUCCCUCUUCGGAGGGAGCAUCAGAG (SEQ ID NO: 156), or a sequence having at least about 96%, at least about 97%, at least about 98%, at least about 99% sequence identity thereto.

In some embodiments, the ERS comprises a sequence of SEQ ID NO: 17, or a sequence having at least about 70% sequence identity thereto, comprising an extended stem loop sequence of SEQ ID NO: 49739 and one or more mutations at positions selected from the group consisting of U11, U24, A29, and A87, wherein the one or more mutations improve the editing ability of the ERS relative to SEQ ID NO: 17.

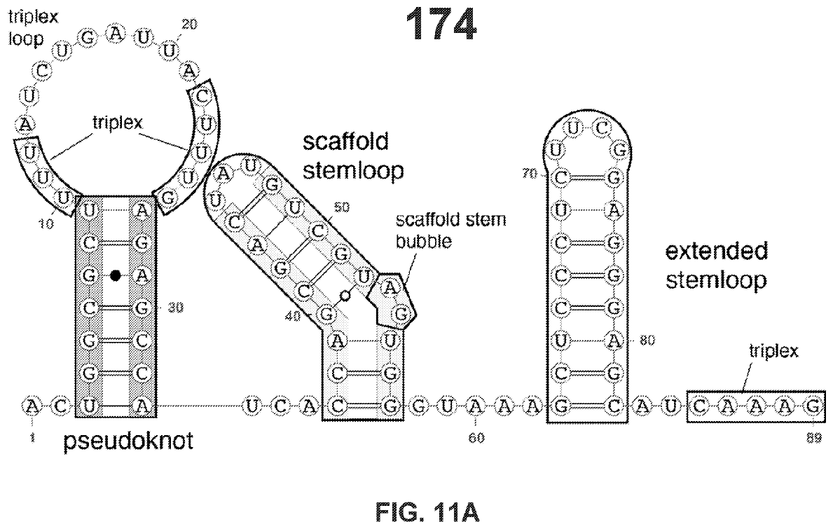

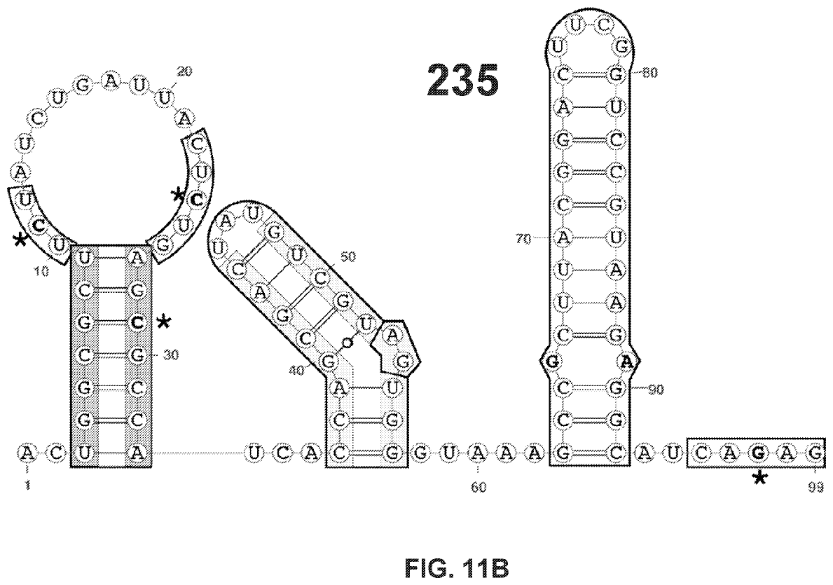

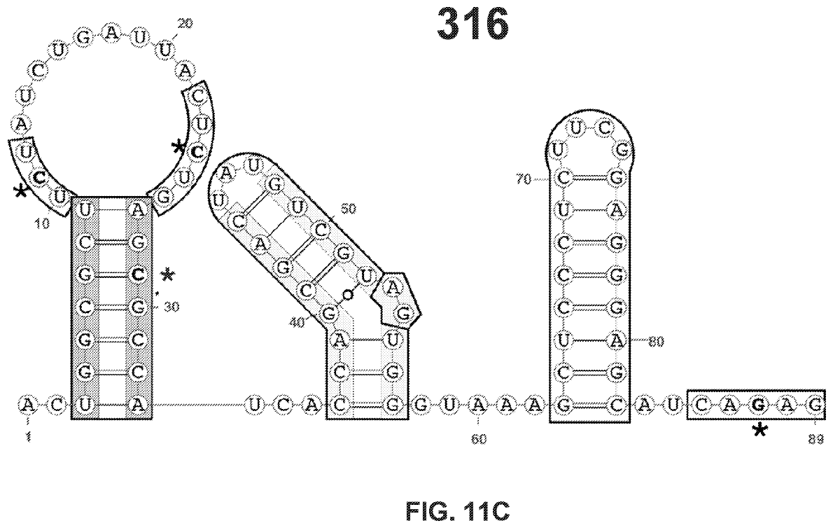

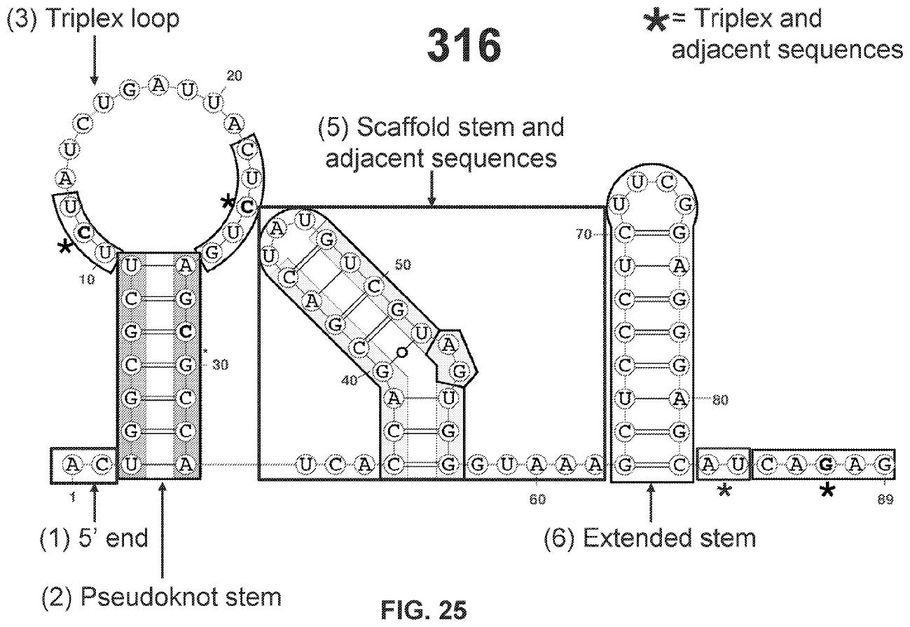

In one embodiment, an ERS scaffold was designed wherein the scaffold 235 sequence (SEQ ID NO: 75) was modified by a domain swap in which the extended stemloop of scaffold variant 174 (SEQ ID NO: 49739) replaced the extended stemloop of the 235 scaffold. In some embodiments, the disclosure provides an ERS comprising a sequence of SEQ ID NO: 75, or a sequence having at least about 70% sequence identity thereto, modified to comprise an extended stem loop sequence of SEQ ID NO: 49739. In some embodiments, the ERS modified to comprise the extended stem loop sequence of SEQ ID NO: 49739 further comprises one or more regions selected from the group consisting of: i) a 5′ end comprising a sequence of AC; ii) a pseudoknot stem I comprising a sequence of UGGCGCU; iii) a triplex loop comprising a sequence of SEQ ID NO: 49736; iv) a pseudoknot stem II comprising a sequence of AGCGCCA; and a triplex region III comprising a sequence of CAGAG. In the foregoing embodiments, the modifications result in the chimeric ERS 316 (see FIG. 11C and FIG. 25), having the sequence ACUGGCGCUUCUAUCUGAUUACUCUGAGCGCCAUCACCAGCGACUAUGUCGUAGU GGGUAAAGCUCCCUCUUCGGAGGGAGCAUCAGAG (SEQ ID NO: 156), having 89 nucleotides in the scaffold, compared with the 99 nucleotides of gRNA variant 235. In some embodiments, the shorter sequence length of the 316 scaffold confers the improvements of a higher fidelity in the ability to create the guide synthetically with the correct and complete sequence, as well as an enhanced ability to be successfully incorporated into an LNP. In addition to improvements in manufacturability, the 316 scaffold was determined to perform comparably or more favorably than gRNA variant 174 in editing assays, as described in the Examples. The resulting 316 scaffold had the further advantage in that the extended stemloop did not contain CpG motifs; an enhanced property described more fully, below. In some embodiments, the 316 scaffold was subjected to chemical modification to create additional ERS, described below. The sequences of the regions of ERS scaffold 316 are presented in Table 3.

TABLE 3
ERS 316 scaffold

| | | SEQ ID |
|---|---|---|
| Region of Scaffold | RNA Sequence* | NO |
| 5′ end | AC | — |
| Pseudoknot stem I | UGGCGCU | — |
| Triplex loop (including triplex | UCUAUCUGAUUACUCUG | 49736 |
| regions I and II) | | |
| Pseudoknot stem II | AGCGCCA | — |
| Linking nucleotides I | UCA | — |
| Scaffold stem loop | CCAGCGACUAUGUCGUAGUGG | 49737 |
| Linking nucleotides II | GUAAA | — |
| Extended stem loop | GCUCCCUCUUCGGAGGGAGC | 49739 |
| Linking nucleotides III | AU | — |
| Triplex region III | CAGAG | — |

*Bases that form the triplex (herein termed triplex regions I-III) are bolded and underlined.

c. Chemically-Modified ERS

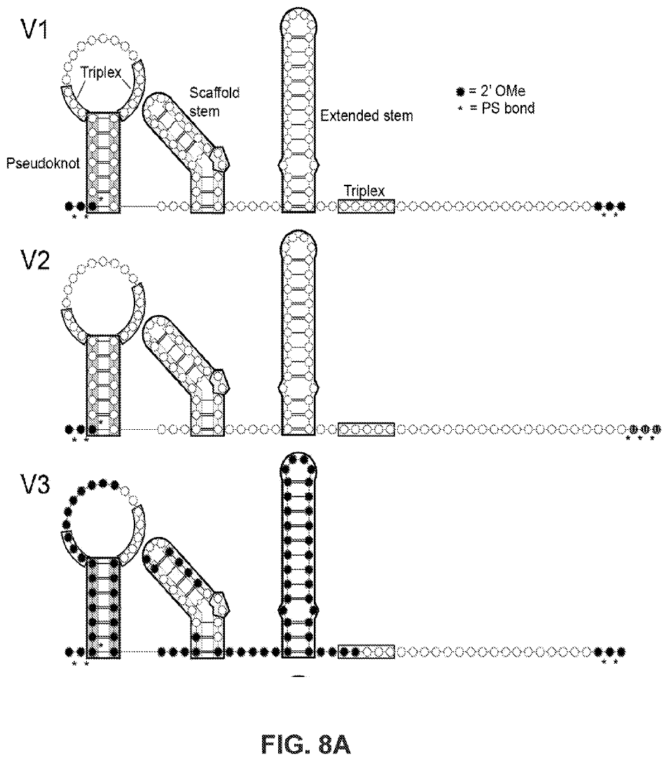

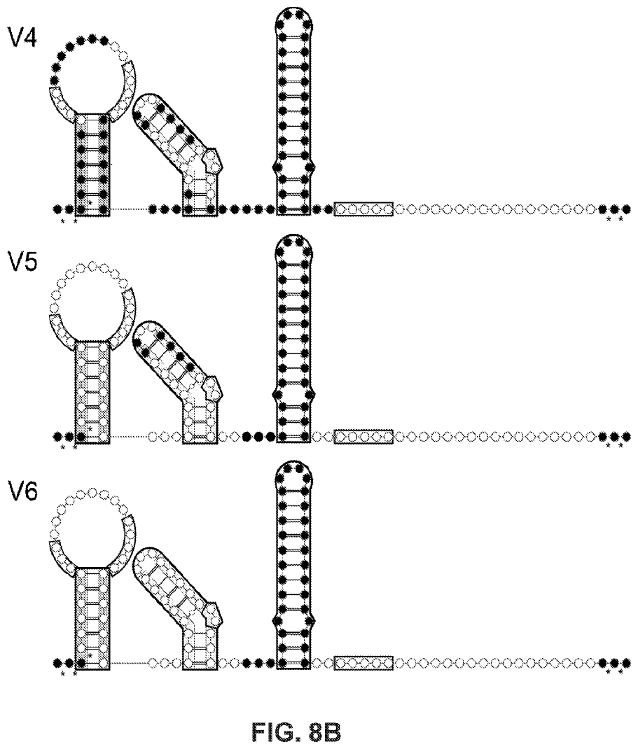

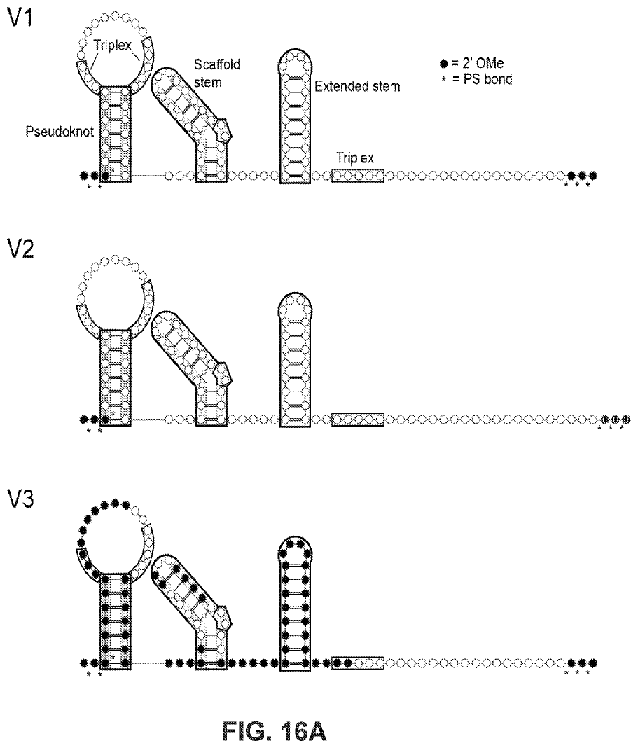

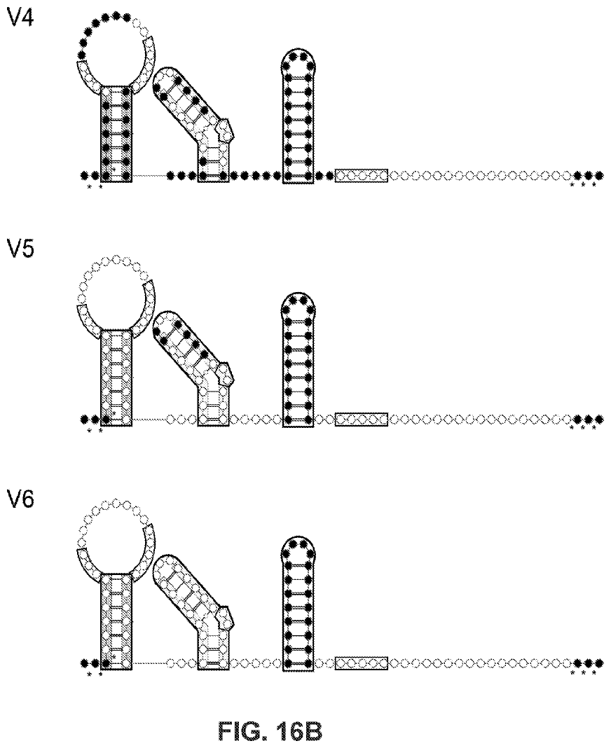

In some embodiments, the present disclosure provides ERS having one or more chemical modifications in order to enhance the chemical stability of ERS. In some cases, the chemically modified ERS are utilized in LNP formations, wherein the ability of the incorporated RNA of the LNP is required to fold and assume and maintain its structural conformation, as well as resist nuclease degradation or induce an immune response when introduced into a target cell environment. Chemical modification of RNAs has been shown to improve stability, increase nuclease resistance by cellular RNase, increase duplex bond formation, and reduce immune responses by the selective modification of the nucleotides, resulting in enhanced editing in CRISPR systems (Basila, M., et al. Minimal 2′-O-methyl phosphorothioate linkage modification pattern of synthetic guide RNAs for increased stability and efficient CRISPR-Cas9 gene editing avoiding cellular toxicity. PLoS ONE 12(11): e0188593 (2017)). In some embodiments, the chemical modification is the addition of a 2′O-methyl group to one or more nucleotides of the sequence of the ERS and linked targeting sequence. In some embodiments, the chemical modification is the addition of a 2′O-methyl group on each terminal end, 5′ and 3′, of the ERS. In some embodiments, the chemical modification is substitution of a phosphorothioate bond between two or more nucleosides of the sequence. In some embodiments, the first 1, 2, or 3 nucleotides of the 5′ end of the scaffold (i.e., A, C, and U in the case of scaffolds 174, 235, and 316) are modified by the addition of a 2′O-methyl group and each of the modified nucleosides is linked to the adjoining nucleoside by a phosphorothioate bond. Similarly, the last 1, 2, or 3 nucleotides of the 3′ end of the targeting sequence linked to the 3′ end of the scaffold are similarly modified to produce an end-protected variant (collectively, the construct with the foregoing modifications termed “vi”). In other embodiments, the 5′ and 3′ ends, as well as nucleotides in select interior regions are similarly modified by the addition of a 2′O-methyl group. In another embodiment, ERS and linked targeting sequence were designed in which a 3′UUU tail was added, in addition to the v1 modifications, to the construct to mimic the termination sequence used in cellular transcription systems and to move the modified nucleotides of the v1 outside of the region of the targeting sequence involved in target recognition (termed “v2”). In another embodiment, ERS were designed in which, in addition to the v1 end-protection modifications, additional 2′OMe modifications were made at nucleotides identified to be potentially modifiable, based on structural analysis of the scaffold (termed “v3”). In another embodiment, ERS were designed in which the 2′OMe modifications of the v3 version in the triplex region of the scaffold were removed to reduce perturbation of the RNA helical structure and maintain backbone flexibility of the resulting scaffold (termed “v4). In another embodiment, ERS were designed in which the modifications included the end-protected modifications of the v1 version and 2′OMe modifications were introduced in the scaffold stem and extended stem regions of the scaffold (termed “v5”). In another embodiment, ERS were designed in which the modifications included the end-protected modifications of the v1 version and 2′OMe modifications were introduced only in the extended stem region of the scaffold (termed “v6”). Schematics of the configurations are show in FIGS. 8A, 8B, 10, 16A and 16B. In some embodiments, the disclosure provides ERS of the v1, v2, v3, v4, v5, v6, v7, v8, or v9 configurations having a sequence selected from the group consisting of the sequences set forth in Table 29 (SEQ ID NOS: 49750-49758, 49760-49768, and 49770-49749) of Example 8 (it being understood that for utilization in the systems of the disclosure, the non-targeting 20 nucleotides at the 3′ end are replaced with a targeting sequence complementary to the target nucleic acid to be modified). In a particular embodiment, the ERS comprises the sequence of SEQ ID NO: 49770 (it being understood that for utilization in the systems of the disclosure, the non-targeting 20 nucleotides at the 3′ end are replaced with a targeting sequence complementary to the target nucleic acid to be modified). In some embodiments, the ERS and linked targeting sequence of the v1, v2, v3, v4, v5, v6, v7, v8, or v9 configurations retain at least about 10%, at least about 20%, at least about 30%, at least about 40%, at least about 50%, at least about 60%, at least about 70%, at least about 80%, or at least about 90% of the editing of a target nucleic acid compared to the unmodified gRNA when assessed in comparable in vitro assays with a CasX nuclease. In some embodiments, the ERS and linked targeting sequence of the vi, v2, v3, v4, v5, v6, v7, v8, or v9 configurations exhibit reduced susceptibility of the ERS to degradation by cellular RNase compared to an unmodified ERS. In some embodiments, the chemically-modified ERS exhibit at least about 10%, at least about 20%, at least about 30%, at least about 40%, at least about 50%, at least about 60% less susceptibility to degradation by cellular RNase compared to an unmodified ERS.

e. CpG Depleted ERS

In the context of use of recombinant adenovirus associated vectors (AAV) for delivery of the ERS and engineered CasX of the embodiments, it was determined that unmethylated CpG dinucleotides in viral DNA can bind TLR9, an endosomal PRR in plasmacytoid dendritic cells (pDCs) and B cells, and result in immune responses in mammalian hosts (Faust, S M, et al. CpG-depleted adeno-associated virus vectors evade immune detection. J. Clinical Invest. 123:2294 (2013)). In particular, CpG dinucleotide motifs in AAV vectors are immunostimulatory because of their high degree of hypomethylation, relative to mammalian CpG motifs, which have a high degree of methylation. Accordingly, reducing the frequency of unmethylated CpG in rAAV vector genomes to a level below the threshold that activates human TLR9 is expected to reduce the immune response to exogenously administered rAAV-based biologics.

In some embodiments, the present disclosure provides ERS that are codon-optimized for depletion of CpG dinucleotides by the substitution of homologous nucleotide sequences from mammalian species, wherein the modified ERS substantially retain the functional property of driving expression of the ERS upon expression in a cell transduced with an AAV comprising the modified ERS. In some embodiments, the present disclosure provides ERS for inclusion in rAAV vectors wherein the encoding sequence for the ERS comprises less than about 10%, less than about 5%, or less than about 1% CpG dinucleotides, and retains the ability to result in transcription of an ERS capable of binding an engineered CasX. In some embodiments, the CpG-depleted ERS is encoded by a DNA sequence comprising a sequence selected from the group consisting of the sequences of Table 38 (SEQ ID NOS: 535-556). In some embodiments, the CpG-depleted ERS comprises a sequence selected from the group consisting of the sequences of Table 38 (SEQ ID NOS: 160-181).

In some embodiments, the administration of a therapeutically effective dose of an rAAV vector comprising the CpG-depleted ERS of the transgene to a subject results in a reduced immune response compared to the immune response of a comparable rAAV vector wherein the ERS has not been codon-optimized for depletion of CpG dinucleotides, wherein the reduced response is determined by the measurement of one or more parameters such as production of antibodies or a delayed-type hypersensitivity to the ERS, or the production of inflammatory cytokines and markers, such as, but not limited to TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF). In some embodiments, the rAAV vector comprising the CpG-depleted ERS of the transgene elicits reduced production of one or more inflammatory markers selected from the group consisting of TLR9, interleukin-1 (IL-1), IL-6, IL-12, IL-18, tumor necrosis factor alpha (TNF-α), interferon gamma (IFNγ), and granulocyte-macrophage colony stimulating factor (GM-CSF) of at least about 10%, at least about 20%, at least about 30%, at least about 40%, at least about 50%, at least about 60%, at least about 80%, or at least about 90% compared to the comparable rAAV that is not CpG depleted, when assayed in a cell-based vitro assay using cells known in the art appropriate for such assays; e.g., monocytes, macrophages, T-cells, B-cells, etc. In a particular embodiment, the rAAV vector comprising the CpG-depleted ERS of the transgene exhibits a reduced activation of TLR9 in hNPCs in an in vitro assay of at least about 10%, at least about 20%, at least about 30%, at least about 40%, at least about 50%, at least about 60%, at least about 80%, or at least about 90% compared to the comparable rAAV that is not CpG depleted.

f. Complex Formation with Class 2, Type V Protein

In some embodiments, upon expression, the ERS is capable of complexing as an RNP with an engineered CasX proteins comprising any one of the sequences of SEQ ID NOS: 247-294, 24916-49628, 49746-49747, and 49871-49873, or a sequence having at least about 50%, at least about 60%, at least about 70%, at least about 80%, at least about 85%, at least about 90%, at least about 91%, at least about 92%, at least about 93%, at least about 94%, at least about 95%, at least about 96%, at least about 97%, at least about 98%, or at least about 99% identity thereto.

In some embodiments, upon expression, the ERS is capable of complexing as an RNP with an engineered CasX protein comprising a pair of mutations as depicted in Table 22, or further variations thereof. In some embodiments, an ERS has an improved ability to form a complex with an engineered CasX protein when compared to a gRNA variant or a reference gRNA, thereby improving its ability to form a cleavage-competent ribonucleoprotein (RNP) complex with the engineered CasX protein. Improving ribonucleoprotein complex formation may, in some embodiments, improve the efficiency with which functional RNPs are assembled. In some embodiments, greater than 90%, greater than 93%, greater than 95%, greater than 96%, greater than 97%, greater than 98% or greater than 99% of RNPs comprising an ERS and its targeting sequence are competent for gene editing of a target nucleic acid.

### IV. Engineered CasX Proteins for Modifying a Target Nucleic Acid

The present disclosure provides engineered CasX nuclease proteins that have utility in genome editing of eukaryotic cells. The engineered CasX nucleases employed in the genome editing systems are Class 2, Type V nucleases. Although members of Class 2, Type V CRISPR-Cas systems have differences, they share some common characteristics that distinguish them from the Cas9 systems. Firstly, the Class 2, Type V nucleases possess a single RNA-guided RuvC domain-containing effector but no HNH domain, and they recognize TC motif PAM 5′ upstream to the target region on the non-targeted strand, which is different from Cas9 systems which rely on G-rich PAM at 3′ side of target sequences. Type V nucleases generate staggered double-stranded breaks distal to the PAM sequence, unlike Cas9, which generates a blunt end in the proximal site close to the PAM. In addition, Type V nucleases degrade ssDNA in trans when activated by target dsDNA or ssDNA binding in cis. In some embodiments, the engineered CasX nucleases of the embodiments recognize a 5′-TC PAM motif and produce staggered ends cleaved solely by the RuvC domain. In some embodiments, the present disclosure provides systems comprising engineered CasX proteins and one or more ERS (eCasX:ERS system) that are specifically designed to modify a target nucleic acid sequence in eukaryotic cells.

The term “CasX protein”, as used herein, refers to a family of proteins, and encompasses all naturally-occurring CasX proteins (“reference CasX”), as well as engineered CasX proteins with sequence modifications possessing one or more improved characteristics relative to a CasX protein from which it was derived, described more fully, below.

The reference CasX, CasX variants (e.g., CasX 515) and engineered CasX proteins of the disclosure comprise the following domains: a non-target strand binding (NTSB) domain, a target strand loading (TSL) domain, a helical I domain (which is further divided into helical I-I and I-II subdomains), a helical II domain, an oligonucleotide binding domain (OBD, which is further divided into OBD-I and OBD-II subdomains), and a RuvC DNA cleavage domain (which is further divided into RuvC-I and II subdomains). In some embodiments, the present disclosure contemplates engineered CasX having multiple mutations in the domains relative to the CasX from which it was derived, wherein the engineered CasX nevertheless retain the ability to form an RNP with an ERS and retains nuclease activity. All such engineered CasX retaining such properties are considered within the scope of the disclosure. In other embodiments, the RuvC domain may be modified or deleted in a catalytically-dead variant.

a. Reference CasX Proteins

For purposes of the disclosure, the sequences of naturally-occurring CasX proteins (referred to herein as a “reference CasX protein”) are provided for illustrative purposes; e.g., identification of domains and subdomains, as well as the ability to reference select amino acid positions. For example, reference CasX proteins can be isolated from naturally occurring prokaryotes, such as Deltaproteobacteria, Planctomycetes, or Candidatus Sungbacteria species. A reference CasX protein is a type II CRISPR/Cas endonuclease belonging to the CasX (interchangeably referred to as Cas12e) family of proteins that interacts with a guide RNA to form a ribonucleoprotein (RNP) complex.

In some cases, a reference CasX protein is isolated or derived from Deltaproteobacter having a sequence of:

| (SEQ ID NO: 1) | |
|---|---|
| 1 | MEKRINKIRK KLSADNATKP VSRSGPMKTL LVRVMTDDLK KRLEKRRKKP EVMPQVISNN |
| 61 | AANNLRMLLD DYTKMKEAIL QVYWQEFKDD HVGLMCKFAQ PASKKIDQNK LKPEMDEKGN |
| 121 | LTTAGFACSQ CGQPLFVYKL EQVSEKGKAY TNYFGRCNVA EHEKLILLAQ LKPEKDSDEA |
| 181 | VTYSLGKFGQ RALDFYSIHV TKESTHPVKP LAQIAGNRYA SGPVGKALSD ACMGTIASFL |
| 241 | SKYQDIIIEH QKVVKGNQKR LESLRELAGK ENLEYPSVTL PPQPHTKEGV DAYNEVIARV |
| 301 | RMWVNLNLWQ KLKLSRDDAK PLLRLKGFPS FPVVERRENE VDWWNTINEV KKLIDAKRDM |
| 361 | GRVFWSGVTA EKRNTILEGY NYLPNENDHK KREGSLENPK KPAKRQFGDL LLYLEKKYAG |
| 421 | DWGKVFDEAW ERIDKKIAGL TSHIEREEAR NAEDAQSKAV LTDWLRAKAS FVLERLKEMD |
| 481 | EKEFYACEIQ LQKWYGDLRG NPFAVEAENR VVDISGFSIG SDGHSIQYRN LLAWKYLENG |
| 541 | KREFYLLMNY GKKGRIRFTD GTDIKKSGKW QGLLYGGGKA KVIDLTFDPD DEQLIILPLA |
| 601 | FGTRQGREFI WNDLLSLETG LIKLANGRVI EKTIYNKKIG RDEPALFVAL TFERREVVDP |
| 661 | SNIKPVNLIG VDRGENIPAV IALTDPEGCP LPEFKDSSGG PTDILRIGEG YKEKQRAIQA |
| 721 | AKEVEQRRAG GYSRKFASKS RNLADDMVRN SARDLFYHAV THDAVLVFEN LSRGFGRQGK |
| 781 | RTFMTERQYT KMEDWLTAKL AYEGLTSKTY LSKTLAQYTS KTCSNCGFTI TTADYDGMLV |
| 841 | RLKKTSDGWA TTLNNKELKA EGQITYYNRY KRQTVEKELS AELDRLSEES GNNDISKWTK |
| 901 | GRRDEALFLL KKRFSHRPVQ EQFVCLDCGH EVHADEQAAL NIARSWLFLN SNSTEFKSYK |
| 961 | SGKQPFVGAW QAFYKRRLKE VWKPNA. |

In some cases, a reference CasX protein is isolated or derived from Planctomycetes having a sequence of:

| (SEQ ID NO: 2) | |
|---|---|
| 1 | MQEIKRINKI RRRLVKDSNT KKAGKTGPMK TLLVRVMTPD LRERLENLRK KPENIPQPIS |
| 61 | NTSRANLNKL LTDYTEMKKA ILHVYWEEFQ KDPVGLMSRV AQPAPKNIDQ RKLIPVKDGN |
| 121 | ERLTSSGFAC SQCCQPLYVY KLEQVNDKGK PHTNYFGRCN VSEHERLILL SPHKPEANDE |
| 181 | LVTYSLGKFG QRALDFYSIH VTRESNHPVK PLEQIGGNSC ASGPVGKALS DACMGAVASF |
| 241 | LTKYQDIILE HQKVIKKNEK RLANLKDIAS ANGLAFPKIT LPPQPHTKEG IEAYNNVVAQ |
| 301 | IVIWVNLNLW QKLKIGRDEA KPLQRLKGFP SFPLVERQAN EVDWWDMVCN VKKLINEKKE |
| 361 | DGKVFWQNLA GYKRQEALLP YLSSEEDRKK GKKFARYQFG DLLLHLEKKH GEDWGKVYDE |
| 421 | AWERIDKKVE GLSKHIKLEE ERRSEDAQSK AALTDWLRAK ASFVIEGLKE ADKDEFCRCE |
| 481 | LKLQKWYGDL RGKPFAIEAE NSILDISGFS KQYNCAFIWQ KDGVKKLNLY LIINYFKGGK |
| 541 | LRFKKIKPEA FEANRFYTVI NKKSGEIVPM EVNFNFDDPN LIILPLAFGK RQGREFIWND |
| 601 | LLSLETGSLK LANGRVIEKT LYNRRTRQDE PALFVALTFE RREVLDSSNI KPMNLIGIDR |
| 661 | GENIPAVIAL TDPEGCPLSR FKDSLGNPTH ILRIGESYKE KQRTIQAAKE VEQRRAGGYS |
| 721 | RKYASKAKNL ADDMVRNTAR DLLYYAVTQD AMLIFENLSR GFGRQGKRTF MAERQYTRME |
| 781 | DWLTAKLAYE GLPSKTYLSK TLAQYTSKTC SNCGFTITSA DYDRVLEKLK KTATGWMTTI |
| 841 | NGKELKVEGQ ITYYNRYKRQ NVVKDLSVEL DRLSEESVNN DISSWTKGRS GEALSLLKKR |
| 901 | FSHRPVQEKF VCLNCGFETH ADEQAALNIA RSWLFLRSQE YKKYQTNKTT GNTDKRAFVE |
| 961 | TWQSFYRKKL KEVWKPAV. |

In some cases, a reference CasX protein is isolated or derived from Candidatus Sungbacteria having a sequence of

| (SEQ ID NO: 3) | |
|---|---|
| 1 | MDNANKPSTK SLVNTTRISD HFGVTPGQVT RVFSFGIIPT KRQYAIIERW FAAVEAARER |
| 61 | LYGMLYAHFQ ENPPAYLKEK FSYETFFKGR PVLNGLRDID PTIMTSAVFT ALRHKAEGAM |
| 121 | AAFHTNHRRL FEEARKKMRE YAECLKANEA LLRGAADIDW DKIVNALRTR LNTCLAPEYD |
| 181 | AVIADFGALC AFRALIAETN ALKGAYNHAL NQMLPALVKV DEPEEAEESP RLRFFNGRIN |
| 241 | DLPKFPVAER ETPPDTETII RQLEDMARVI PDTAEILGYI HRIRHKAARR KPGSAVPLPQ |
| 301 | RVALYCAIRM ERNPEEDPST VAGHFLGEID RVCEKRRQGL VRTPFDSQIR ARYMDIISFR |
| 361 | ATLAHPDRWT EIQFLRSNAA SRRVRAETIS APFEGFSWTS NRTNPAPQYG MALAKDANAP |
| 421 | ADAPELCICL SPSSAAFSVR EKGGDLIYMR PTGGRRGKDN PGKEITWVPG SFDEYPASGV |
| 481 | ALKLRLYFGR SQARRMLINK TWGLLSDNPR VFAANAELVG KKRNPQDRWK LFFHMVISGP |
| 541 | PPVEYLDFSS DVRSRARTVI GINRGEVNPL AYAVVSVEDG QVLEEGLLGK KEYIDQLIET |
| 601 | RRRISEYQSR EQTPPRDLRQ RVRHLQDTVL GSARAKIHSL IAFWKGILAI ERLDDQFHGR |
| 661 | EQKIIPKKTY LANKTGFMNA LSFSGAVRVD KKGNPWGGMI EIYPGGISRT CTQCGTVWLA |
| 721 | RRPKNPGHRD AMVVIPDIVD DAAATGFDNV DCDAGTVDYG ELFTLSREWV RLTPRYSRVM |
| 781 | RGTLGDLERA IRQGDDRKSR QMLELALEPQ PQWGQFFCHR CGFNGQSDVL AATNLARRAI |
| 841 | SLIRRLPDTD TPPTP. |

b. Engineered CasX Proteins

The present disclosure provides highly-modified engineered CasX proteins having multiple mutations relative to a reference CasX or to one or more CasX variant proteins; e.g., CasX 515 or the CasX proteins of Table 9 (SEQ ID NOS: 492-500). The mutations can be in one or more domains of the parental CasX from which the engineered CasX was derived. The CasX domains and their positions, relative to reference CasX SEQ ID NOS: 1 and 2 are presented in Tables 4 and 5.

TABLE 4
Domain coordinates in Reference CasX proteins

| | Coordinates in | Coordinates in |
|---|---|---|
| Domain Name | SEQ ID NO: 1 | SEQ ID NO: 2 |
| OBD-I | 1-55 | 1-57 |
| helical I-I | 56-99 | 58-101 |
| NTSB | 100-190 | 102-191 |
| helical I-II | 191-331 | 192-332 |
| helical II | 332-508 | 333-500 |
| OBD-II | 509-659 | 501-646 |
| RuvC-I | 660-823 | 647-810 |
| TSL | 824-933 | 811-920 |
| RuvC-II | 934-986 | 921-978 |

TABLE 5
Exemplary Domain Sequences in Reference CasX proteins
Deltaproteobacter sp. (reference CasX of SEQ ID NO: 1)

| SEQ | | |
|---|---|---|
| ID | Domain | Sequence |
| 229 | OBD-I | EKRINKIRKKLSADNATKPVSRSGPMKTLLVRVMTDDLKKRLEKRRKKPEVMPQ |
| 230 | helical I-I | VISNNAANNLRMLLDDYTKMKEAILQVYWQEFKDDHVGLMCKFA |
| 231 | NTSB | QPASKKIDQNKLKPEMDEKGNLTTAGFACSQCGQPLFVYKLEQVSEKGKAYTNYFGRCNV |
| | | AEHEKLILLAQLKPEKDSDEAVTYSLGKFGQ |
| 232 | helical I-II | RALDFYSIHVTKESTHPVKPLAQIAGNRYASGPVGKALSDACMGTIASFLSKYQDIIIEH |
| | | QKVVKGNQKRLESLRELAGKENLEYPSVTLPPQPHTKEGVDAYNEVIARVRMWVNLNLWQ |
| | | KLKLSRDDAKPLLRLKGFPSF |
| 233 | helical II | PVVERRENEVDWWNTINEVKKLIDAKRDMGRVFWSGVTAEKRNTILEGYNYLPNENDHKK |
| | | REGSLENPKKPAKRQFGDLLLYLEKKYAGDWGKVEDEAWERIDKKIAGLTSHIEREEARN |
| | | AEDAQSKAVLTDWLRAKASFVLERLKEMDEKEFYACEIQLQKWYGDLRG NPFAVEAE |
| 234 | OBD-II | NRVVDISGESIGSDGHSIQYRNLLAWKYLENGKREFYLLMNYGKKGRIRFTDGTDIKKSG |
| | | KWQGLLYGGGKAKVIDLTFDPDDEQLIILPLAFGTRQGREFIWNDLLSLETGLIKLANGR |
| | | VIEKTIYNKKIGRDEPALFVALTFERREVVD |
| 235 | RuvC-I | PSNIKPVNLIGVDRGENIPAVIALTDPEGCPLPEFKDSSGGPTDILRIGEGYKEKQRAIQ |
| | | AAKEVEQRRAGGYSRKFASKSRNLADDMVRNSARDLFYHAVTHDAVLVFENLSRGFGRQG |
| | | KRTFMTERQYTKMEDWLTAKLAYEGLTSKTYLSKTLAQYTSKTC |
| 236 | TSL | SNCGFTITTADYDGMLVRLKKTSDGWATTLNNKELKAEGQITYYNRYKROTVEKELSAEL |
| | | DRLSEESGNNDISKWTKGRRDEALFLLKKRFSHRPVQEQFVCLDCGHEVH |
| 237 | RuvC-II | ADEQAALNIARSWLFLN SNSTEFKSYKSGKQPFVGAWQAFYKRRLKEVWKPNA |

Planctomycetes sp. (Reference CasX of SEQ ID NO: 2)

| SEQ | | |
|---|---|---|
| ID | Domain | Sequence |
| 238 | OBD-I | QEIKRINKIRRRLVKDSNTKKAGKTGPMKTLLVRVMTPDLRERLENLRKKPENIPQ |
| 239 | helical I-I | PISNTSRANLNKLLTDYTEMKKAILHVYWEEFQKDPVGLMSRVA |
| 240 | NTSB | QPAPKNIDQRKLIPVKDGNERLTSSGFACSQCCQPLYVYKLEQVNDKGKPHTNYFGRCNV |
| | | SEHERLILLSPHKPEANDELVTYSLGKFGQ |
| 241 | helical I-II | RALDFYSIHVTRESNHPVKPLEQIGGNSCASGPVGKALSDACMGAVASFLTKYQDIILEH |
| | | QKVIKKNEKRLANLKDIASANGLAFPKITLPPQPHTKEGIEAYNNVVAQIVIWVNLNLWQ |
| | | KLKIGRDEAKPLQRLKGFPSF |
| 242 | helical II | PLVERQANEVDWWDMVCNVKKLINEKKEDGKVFWQNLAGYKRQEALLPYLSSEEDRKKGK |
| | | KFARYQFGDLLLHLEKKHGEDWGKVYDEAWERIDKKVEGLSKHIKLEEERRSEDAQSKAA |
| | | LTDWLRAKASFVIEGLKEADKDEFCRCELKLOKWYGDLRGKPFAIEAE |
| 243 | OBD-II | NSILDISGFSKQYNCAFIWQKDGVKKLNLYLIINYFKGGKLRFKKIKPEAFEANRFYTVI |
| | | NKKSGEIVPMEVNENFDDPNLIILPLAFGKROGREFIWNDLLSLETGSLK |
| | | LANGRVIEKTLYNRRTRQDEPALFVALTFERREVLD |
| 244 | RuvC-I | SSNIKPMNLIGIDRGENIPAVIALTDPEGCPLSRFKDSLGNPTHILRIGESYKEKORTIQ |
| | | AAKEVEQRRAGGYSRKYASKAKNLADDMVRNTARDLLYYAVTQDAMLIFENLSRGFGRQG |
| | | KRTEMAERQYTRMEDWLTAKLAYEGLPSKTYLSKTLAQYTSKTC |
| 245 | TSL | SNCGFTITSADYDRVLEKLKKTATGWMTTINGKELKVEGQITYYNRYKRQNVVKDLSVEL |
| | | DRLSEESVNNDISSWTKGRSGEALSLLKKRFSHRPVQEKFVCLNCGFETH |
| 246 | RuvC-II | ADEQAALNIARSWLFLRSQEYKKYQTNKTTGNTDKRAFVETWQSFYRKKLKEVWKPAV |

Mutations can be introduced in any one or combinations of domains of the CasX variant to result in an engineered CasX. These alterations can be amino acid insertions, deletions, substitutions, or any combinations thereof. Any amino acid can be substituted for any other amino acid in the substitutions described herein. The substitution can be a conservative substitution (e.g., a basic amino acid is substituted for another basic amino acid). The substitution can be a non-conservative substitution (e.g., a basic amino acid is substituted for an acidic amino acid or vice versa). For example, a proline in a CasX protein can be substituted for any of arginine, histidine, lysine, aspartic acid, glutamic acid, serine, threonine, asparagine, glutamine, cysteine, glycine, alanine, isoleucine, leucine, methionine, phenylalanine, tryptophan, tyrosine or valine to generate an engineered CasX protein of the disclosure. In some embodiments, an engineered CasX comprises two mutations relative to the CasX protein from which it was derived. In some embodiments, an engineered CasX comprises three mutations relative to the CasX protein from which it was derived. In some embodiments, an engineered CasX comprises 2, 3, 4, 5, 6, 7, 8, 9, 10 or more mutations relative to the CasX protein from which it was derived. In some embodiments, the 2, 3, 4, 5, 6, 7, 8, 9, 10 or more mutations are made in locations of the CasX protein sequence separated from one another. In other embodiments, the 2, 3, 4, 5, 6, 7, 8, 9, 10 or more mutations can be made in adjacent amino acids in the CasX protein sequence. In some embodiments, an engineered CasX comprises two or more mutations relative to two or more different CasX proteins from which they were derived. The methods utilized for the design and creation of the engineered CasX are described below, including the methods of the Examples.

Suitable mutagenesis methods for generating engineered CasX proteins of the disclosure may include, for example, random mutagenesis, site-directed mutagenesis, Markov Chain Monte Carlo (MCMC)-directed evolution, staggered extension PCR, gene shuffling, rational design, or domain swapping (described in PCT/US2021/061673 and WO2020247882A1, incorporated by reference herein). In some embodiments, the engineered CasX are designed, for example by selecting multiple desired mutations in a CasX variant identified, for example, using the approaches described in the Examples. In certain embodiments, the activity of the CasX variant protein prior to mutagenesis is used as a benchmark against which the activity of one or more resulting engineered CasX are compared, thereby measuring improvements in function of the engineered CasX.

In some embodiments of the engineered CasX described herein, the approach to design the engineered CasX utilizes a directed evolution method adapted from a Markov Chain Monte Carlo (MCMC)-directed evolution simulation (Biswas N., et al. Coupled Markov Chain Monte Carlo for high-dimensional regression with Half-t priors. arViV: 2012.04798v2 (2021)), as described in Example 1.

In further iterations of the generation of the engineered CasX proteins, a variant CasX protein can be mutagenized to generate sequences that are screened to identity engineered CasX having improved or enhanced characteristics. Exemplary methods used to generate and evaluate engineered CasX derived from other CasX proteins are described in the Examples (e.g., CasX 515), which were created by introducing modifications to the encoding sequence resulting in amino acid substitutions, deletions, or insertions at one or more positions in one or more domains of the parental CasX protein. In some embodiments, the resulting mutagenized sequences are screened to identify those having enhanced nuclease activity. In other embodiments, the mutagenized sequences are screened to identify those having enhanced editing specificity and reduced off-target editing. In other embodiments, the mutagenized sequences are screened to identify those having enhanced PAM utilization; i.e., the ability to utilize non-canonical PAM sequences. In still other embodiments, the mutagenized sequences are screened to identify those having enhanced properties of any two or three of the foregoing categories; i.e., nuclease activity, specificity (reduced off-target editing), and PAM utilization. In other embodiments, libraries of sequence variants having one, two, three or more mutations at select positions relative to a parental CasX protein can be generated and screened in assays such as an E. coli CcdB toxin assay or a multiplexed pooled approach using a PASS assay to identify those engineered CasX that had enhanced nuclease activity, enhanced specificity, and/or increased PAM utilization compared to the cleavage of the E. coli nucleic acid compared to the parental CasX protein, as described in Examples 5-7. The domain sequences of CasX 515 are presented in Table 7.

Any changes in the amino acid sequence of a CasX variant protein from which the engineered CasX was derived and that leads to an improved characteristic of the engineered CasX protein is considered an engineered CasX protein of the disclosure, provided the engineered CasX retains the ability to form an RNP with a gRNA or ERS and retains nuclease activity. In some embodiments, the improved characteristic is one or more of improved editing activity of the target nucleic acid, improved editing specificity for the target nucleic acid, improved editing specificity ratio for the target nucleic acid, decreased off-target editing, increased percentage of a eukaryotic genome that can be efficiently edited, improved ability to form cleavage-competent RNP with an ERS, and improved stability of an RNP complex. In some embodiments, the improved characteristic is at least about 0.1-fold improved, at least about 0.5-fold improved, at least about 1-fold improved, at least about 1-fold improved, at least about 1-fold improved, at least about 1.5-fold improved, at least about 2-fold improved, at least about 3-fold improved, at least about 4-fold improved, at least about 5-fold improved, at least about 6-fold improved, at least about 7-fold improved, at least about 8-fold improved, at least about 9-fold improved, at least about 10-fold improved, or any integer in between the foregoing. In some embodiments, the engineered CasX protein comprises between 700 and 1200 amino acids, between 800 and 1100 amino acids, or between 900 and 1000 amino acids.

In some embodiments, the disclosure provides engineered CasX derived from CasX 515 (SEQ ID NO: 49699) comprising two or more modifications; an insertion, a deletion, or a substitution of amino acid(s) in one or more domains (see Table 7 for CasX 515 domain sequences). In some embodiments, the disclosure provides engineered CasX proteins comprising a pair of mutations relative to CasX 515 (SEQ ID NO: 49699) as depicted in Table 22, or further variations thereof. In some embodiments, an engineered CasX comprising two or more modifications comprises a sequence selected from the group consisting of SEQ ID NOS: 247-294, 27857-49628, 49746-49747, and 49871-49873, or a sequence having at least about 70%, at least about 80%, at least about 90%, or at least about 95%, or at least about 95%, or at least about 96%, or at least about 97%, or at least about 98%, or at least about 99% sequence identity thereto. In a particular approach, as detailed in Example 7, single mutations of CasX 515 (SEQ ID NO: 49699) that demonstrated enhanced activity and/or specificity, were selected based on locations deemed to be potentially complementary, and combined (i.e., having two or three mutations) to make engineered CasX that were then screened for activity and specificity in in vitro assays. The positions of the mutations within domains of CasX are described in detail in Table 21 in the Examples, below. In some embodiments, the engineered CasX comprises an OBD-I comprising an amino acid sequence comprising one or more mutations relative to the sequence of SEQ ID NO: 295. In some embodiments, the engineered CasX comprises an OBD-I comprising one or more mutations relative to the sequence of SEQ ID NO: 295 selected from the group consisting of an I3G substitution, an insertion of a G at position 4, a K4G substitution, an insertion of a G at position 5, a K8G substitution, an insertion of an R at position 26, and a R34P substitution. In some embodiments, the engineered CasX comprises an OBD-I comprising a sequence selected from the group consisting of SEQ ID NOS: 295, 49800, 49803-49808, and 49822-49833, or a sequence having at least about 90%, at least about 95%, at least about 98%, at least about 99% sequence identity thereto. In some embodiments, the engineered CasX comprises a helical I-I domain comprising an amino acid sequence comprising one or more mutations relative to the sequence of SEQ ID NO: 296. In some embodiments, the engineered CasX comprises a helical I-I domain comprising an R7Q substitution relative to the amino acid sequence of SEQ ID NO: 296. In some embodiments, the engineered CasX comprises a helical I-I domain comprises a sequence selected from the group consisting of SEQ ID NOS: 296 and 49809, or a sequence having at least about 90%, at least about 95%, at least about 98%, or at least about 99% sequence identity thereto. In some embodiments, the engineered CasX comprises an NTSB domain comprising an amino acid sequence comprising one or more mutations relative to the sequence of SEQ ID NO: 297. In some embodiments, the engineered CasX comprises an NTSB domain comprising one or more mutations relative to the sequence of SEQ ID NO: 297 selected from the group consisting of an L68K substitution, an L68Q substitution, an A70Y substitution, an A70D substitution, and an A70S substitution. In some embodiments, the engineered CasX comprises an NTSB domain comprising a sequence selected from the group consisting of SEQ ID NOS: 297, 49802, 49810, 49811, 49812, 49818, and 49835-49840, or a sequence having at least about 90%, at least about 95%, at least about 98%, or at least about 99% sequence identity thereto. In some embodiments, the engineered CasX comprises a helical I-II domain comprising an amino acid sequence comprising one or more mutations relative to the sequence of SEQ ID NO: 298. In some embodiments, the engineered CasX comprises a helical I-II domain comprising one or more mutations relative to the sequence of SEQ ID NO: 298 selected from the group consisting of a G32T substitution, an M112T substitution, and an M112W substitution. In some embodiments, the engineered CasX comprises a helical I-II domain comprising a sequence selected from the group consisting of SEQ ID NOS: 298, 49801, 49813-49814, and 49842, or a sequence having at least about 90%, at least about 95%, at least about 98%, or at least about 99% sequence identity thereto. In some embodiments, the engineered CasX comprises a helical II domain comprising an amino acid sequence comprising one or more mutations relative to the sequence of SEQ ID NO: 299. In some embodiments, the engineered CasX comprises a helical II domain comprising one or more mutations relative to the sequence of SEQ ID NO: 299 selected from the group consisting of a Y65T substitution and an E148D substitution. In some embodiments, the engineered CasX comprises a helical II domain comprising a sequence selected from the group consisting of SEQ ID NOS: 299, 49815-49816, and 49843, or a sequence having at least about 90%, at least about 95%, at least about 98%, or at least about 99% sequence identity thereto. In some embodiments, the engineered CasX comprises a RuvC-I domain comprising an amino acid sequence comprising one or more mutations relative to the sequence of SEQ ID NO: 301. In some embodiments, the engineered CasX comprises a RuvC-I domain comprising an S51R substitution relative to the sequence of SEQ ID NO: 301. In some embodiments, the engineered CasX comprises a RuvC-I domain comprising a sequence selected from the group consisting of SEQ ID NOS: 301 and 49821, or a sequence having at least about 90%, at least about 95%, at least about 98%, or at least about 99% sequence identity thereto. In some embodiments, the engineered CasX comprises a TSL domain comprising an amino acid sequence comprising one or more mutations relative to the sequence of SEQ ID NO: 302. In some embodiments, the engineered CasX comprises a TSL domain comprising one or more mutations relative to the sequence of SEQ ID NO: 302 selected from the group consisting of a V15M substitution, a T76D substitution, and an S80Q substitution. In some embodiments, the engineered CasX comprises a TSL domain comprising a sequence selected from the group consisting of SEQ ID NOS: 302, 49817, 49819, 49820, and 49844-49846, or a sequence having at least about 90%, at least about 95%, at least about 98%, or at least about 99% sequence identity thereto. In some embodiments, the engineered CasX comprises an OBD-II domain comprising the sequence of SEQ ID NO: 300, or a sequence having at least about 90%, at least about 95%, at least about 98%, or at least about 99% sequence identity thereto. In some embodiments, the engineered CasX comprises a RuvC-II domain comprising the sequence of SEQ ID NO: 303, or a sequence having at least about 90%, at least about 95%, at least about 98%, or at least about 99% sequence identity thereto. In some embodiments, the engineered CasX comprises two or more mutations selected from the group consisting of 4.I.G & 64.R.Q, 4.I.G & 169.L.K, 4.I.G & 169.L.Q, 4.I.G & 171.A.D, 4.I.G & 171.A.Y, 4.I.G & 171.A.S, 4.I.G & 224.G.T, 4.I.G & 304.M.T, 4.G & 398.Y.T, 4.I.G & 826.V.M, 41G & 887.T.D, 4.I.G & 891.S.Q, 5.-.G & 64.R.Q, 5.-.G & 169.L.K, 5.-.G & 169.L.Q, 5.-.G & 171.A.D, 5.-.G & 171.A.Y, 5.-.G & 171.A.S, 5.-.G & 224.G.T, 5.-.G & 304.M.T, 5.-.G & 398.Y.T, 5.-.G & 826.V.M, 5.-.G & 887.T.D, 5.-.G & 891.S.Q, 9.K.G & 64.R.Q, 9.K.G & 169.L.K, 9.K.G & 169.L.Q, 9.K.G & 171.A.D, 9.K.G & 171.A.Y, 9.K.G & 171.A.S, 9.K.G & 224.G.T, 9.K.G & 304.M.T, 9.K.G & 398.Y.T, 9.K.G & 826.V.M, 9.K.G & 887.T.D, 9.K.G & 891.S.Q, 27.-.R & 64.R.Q, 27.-.R & 169.L.K, 27.-.R & 169.L.Q, 27.-.R & 171.A.D, 27.-.R & 171.A.Y, 27.-.R & 171.A.S, 27.-.R & 224.G.T, 27.-.R & 304.M.T, 27.-.R & 398.Y.T, 27.-.R & 826.V.M, 27.-.R & 887.T.D, 27.-.R & 891.S.Q, 35.R.P & 64.R.Q, 35.R.P & 169.L.K, 35.R.P & 169.L.Q, 35.R.P & 171.A.D, 35.R.P & 171.A.Y, 35.R.P & 171.A.S, 35.R.P & 224.G.T, 35.R.P & 304.M.T, 35.R.P & 398.Y.T, 35.R.P & 826.V.M, 35.R.P & 887.T.D, 35.R.P & 891.S.Q, 887.T.D & 891.S.Q, 64.R.Q & 169.L.K, 64.R.Q & 169.L.Q, 64.R.Q & 171.A.D, 64.R.Q & 171.A.Y, 64.R.Q & 171.A.S, 64.R.Q & 224.G.T, 64.R.Q & 304.M.T, 64.R.Q & 398.Y.T, 64.R.Q & 826.V.M, 64.R.Q & 887.T.D, 64.R.Q & 891.S.Q, 169.L.K & 171.A.D, 169.L.K & 171.A.Y, 169.L.K & 171.A.S, 169.L.K & 224.G.T, 169.L.K & 304.M.T, 169.L.K & 398.Y.T, 169.L.K & 826.V.M, 169.L.K & 887.T.D, 169.L.K & 891.S.Q, 169.L.Q & 171.A.D, 169.L.Q & 171.A.Y, 169.L.Q & 171.A.S, 169.L.Q & 224.G.T, 169.L.Q & 304.M.T, 169.L.Q & 398.Y.T, 169.L.Q & 826.V.M, 169.L.Q & 887.T.D, 169.L.Q & 891.S.Q, 171.A.D & 224.G.T, 171.A.D & 304.M.T, 171.A.D & 398.Y.T, 171.A.D & 826.V.M, 171.A.D & 887.T.D, 171.A.D & 891.S.Q, 171.A.Y & 224.G.T, 171.A.Y & 304.M.T, 171.A.Y & 398.Y.T, 171.A.Y & 826.V.M, 171.A.Y & 887.T.D, 171.A.Y & 891.S.Q, 171.A.S & 224.G.T, 171.A.S & 304.M.T, 171.A.S & 398.Y.T, 171.A.S & 826.V.M, 171.A.S & 887.T.D, 171.A.S & 891.S.Q, 4.I.G & 35.R.P, 224.G.T & 304.M.T, 224.G.T & 398.Y.T, 224.G.T & 826.V.M, 224.G.T & 887.T.D, 224.G.T & 891.S.Q, 5.-.G & 35.R.P, 4.I.G & 27.-.R, 304.M.T & 398.Y.T, 304.M.T & 826.VM, 304.M.T & 887.T.D, 304.M.T & 891.S.Q, 9.K.G & 35.R.P, 5.-.G & 27.-.R, 4.I.G & 9.K.G, 398.Y.T & 826.V.M, 398.Y.T & 887.T.D, 398.Y.T & 891.S.Q, 27.-.R & 35.R.P, 9.K.G & 27.-.R, 5.-.G & 9.K.G, 41G & 5.-.G, 826.V.M & 887.T.D, 826.V.M & 891.S.Q, 5.K.G & 27.-.R, 5.K.G & 169.L.K, 5.K.G & 171.A.D, 5.K.G & 304.M.T, 5.K.G & 398.Y.T, 5.K.G & 891.S.Q, 6.-.G & 27.-.R, 6.-.G & 169.L.K, 6.-.G & 171.A.D, 6.-.G & 304.M.T, 6.-.G & 398.Y.T, 6.-.G & 891.S.Q, 304.M.W & 27.-.R, 304.M.W & 169.L.K, 304.M.W & 171.A.D, 304.M.W & 398.Y.T, 304.M.W & 891.S.Q, 481.E.D & 27.-.R, 481.E.D & 169.L.K, 481.E.D & 171.A.D, 481.E.D & 304.M.T, 481.E.D & 398.Y.T, 481.E.D & 891.S.Q, 698.S.R & 27.-.R, 698.S.R & 169.L.K, 698.S.R & 171.A.D, 698.S.R & 304.M.T, 698.S.R & 398.Y.T, and 698.S.R & 891.S.Q, as provided in Table 22, wherein the position of the mutations is relative to the CasX sequence of SEQ ID NO: 49699. In some embodiments, the engineered CasX comprises two or more mutations from Table 22, wherein the two or more mutations result in an improved characteristic compared to unmodified CasX 515 (SEQ ID NO: 49699). In some embodiments, the improved characteristics is determined compared to the unmodified parental CasX 515 in an in vitro assay under comparable conditions. In some embodiments, the improved characteristic is decreased off-target editing, e.g., as shown in Table 27. In some embodiments, the improved characteristic is increased on-target editing, e.g., as shown in Table 25.

In some embodiments, the engineered CasX comprises three mutations in the sequence of CasX 515 (SEQ ID NO: 49699), wherein the three mutations are selected from the group consisting of 27.-.R, 169.L.K, and 329.G.K; 27.-.R, 171.A.D, and 224.G.T; and 35.R.P, 171.A.Y, and 304.M.T, wherein the mutations result in an improved characteristic compared to unmodified CasX 515.

In some embodiments, an engineered CasX selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 exhibits improved editing activity compared to the unmodified parental CasX 515. In some embodiments, the improved characteristics is determined compared to the unmodified parental CasX 515 in an in vitro assay under comparable conditions.

In some embodiments, an engineered CasX selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 exhibits improved editing specificity compared to the unmodified parental CasX 515, In some embodiments, the improved characteristics is determined compared to the unmodified parental CasX 515 in an in vitro assay under comparable conditions.

In some embodiments, an engineered CasX selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 exhibits improved activity and specificity compared to the unmodified parental CasX 515. In some embodiments, the improved characteristics is determined compared to the unmodified parental CasX 515 in an in vitro assay under comparable conditions.

In some embodiments, an engineered CasX selected from the group consisting of SEQ ID NOS: 27865, 27952, 27954, 27955, 27958, 27959, 27973, 28009, 28018, 28048, 28101, 28123, 28137, 28285, 28296, 28301, 28305, 28314, 28323, 28368, 28369, 28370, 28378, 28387, 28438, 28447, 28477, 28481, 28498, 28515, 28524, 28532, 28661, 28799, 28925, 29022, 29266, 29308, 29371, 29560, 29749, 29917, 30888, 31244, 33212, 33512, 34088, 34870, 35422, 35507, 43373, 49872, and 49873 exhibits improved specificity ratio compared to the unmodified parental CasX 515. In some embodiments, the improved characteristics is determined compared to the unmodified parental CasX 515 in an in vitro assay under comparable conditions.

In some embodiments, an engineered CasX selected from the group consisting of SEQ ID NOS: 27952, 27958, 28101, 28123, 28137, 28285, 28368, 28370, 28378, 28387, 28438, 28799, 28925, 29022, 29308, 29749, 29917, 30888, 34870, 43373, and 49873 exhibits improved editing activity and improved editing specificity compared to the unmodified parental CasX 515. In some embodiments, the improved characteristics is determined compared to the unmodified parental CasX 515 in an in vitro assay under comparable conditions.

In some embodiments, an engineered CasX selected from the group consisting of SEQ ID NOS: 27952, 27958, 28036, 28101, 28123, 28137, 28285, 28368, 28370, 28378, 28387, 28438, 28499, 28799, 28925, 29011, 29022, 29308, 29749, 29917, 30888, 34870, 35402, 35512, 43373, and 49873 exhibits improved editing activity and improved editing specificity ratio compared to the unmodified parental CasX 515. In some embodiments, the improved characteristics is determined compared to the unmodified parental CasX 515 in an in vitro assay under comparable conditions.

In some embodiments, the foregoing characteristics of the engineered CasX are improved be at least about 0.1-fold, at least about 0.5-fold, at least about 1-fold, at least about 2-fold, at least about 4-fold, at least about 5-fold, at least about 6-fold, at least about 7-fold, at least about 8-fold, at least about 9-fold, or at least about 10-fold improved compared to the unmodified parental CasX 515.

In some embodiments, the engineered CasX protein comprises, from N- to C-terminus, an OBD-I domain, a helical I-I domain, an NTSB domain, a helical I-II domain, a helical II domain, an OBD-II, a RuvC-I domain, a TSL domain, and a RuvC-II domain, with each domain comprising a sequence as set forth in Table 23, or a sequence having at least about 90%, or at least about 95% sequence identity thereto. In some embodiments, the engineered CasX protein comprising a pair of mutations as depicted in Table 22, or further variations thereof, and demonstrates increased on-target editing activity or decreased off-target activity (specificity) compared to the unmodified parental CasX variant 515, when assayed in an in vitro assay under comparable conditions.

As described in the Examples, an engineered CasX termed “CasX 812” was generated. As described in Example 2, CasX 812 was generated via a glycine-to-lysine substitution at position 329 in CasX 515, within the helical I-II domain. CasX 812 demonstrated an improved specificity relative to CasX 515 in the pooled activity and specificity (PASS) assays described in Example 2 and Example 6. The amino acid sequences of the domains of CasX 812 are provided in Table 13 in the Examples. Accordingly, in some embodiments, the disclosure provides an engineered CasX comprising an amino acid substitution at position 329 relative to a CasX 515 protein comprising amino acid sequence of SEQ ID NO: 49699. In some embodiments, the engineered CasX comprises a mutation in the helical I-II domain relative to CasX 515. In some embodiments, the engineered CasX comprises a mutation at position G137 relative to the helical I-II domain of CasX 515. In some embodiments, the engineered CasX comprises a helical I-II domain sequence of SEQ ID NO: 298, or a sequence having at least about 90%, or at least about 95% sequence identity thereto, comprising an amino acid substitution of position G137 relative to the sequence of SEQ ID NO: 298. In some embodiments, the substituted position comprises a hydrophilic amino acid residue. In some embodiments, the hydrophilic amino acid residue is a lysine residue. In some embodiments, hydrophilic amino acid residues an asparagine residue. In some embodiments, the engineered CasX comprises an OBD-I domain comprising the amino acid sequence of SEQ ID NO: 295, or a sequence having at least about 90%, or at least about 95% sequence identity thereto. In some embodiments, the engineered CasX comprises a helical I-I domain comprising the amino acid sequence of SEQ ID NO: 296, or a sequence having at least about 90%, or at least about 95% sequence identity thereto. In some embodiments, the engineered CasX comprises an NTSB domain comprising the amino acid sequence of SEQ ID NO: 297, or a sequence having at least about 90%, or at least about 95% sequence identity thereto. In some embodiments, the engineered CasX comprises a helical I-II domain comprising the amino acid sequence of SEQ ID NO: 49847, or a sequence having at least about 90%, or at least about 95% sequence identity thereto. In some embodiments, the engineered CasX comprises an OBD-II domain comprising the amino acid sequence of SEQ ID NO: 300, or a sequence having at least about 90%, or at least about 95% sequence identity thereto. In some embodiments, the engineered CasX comprises a RuvC-I domain comprising the amino acid sequence of SEQ ID NO: 301, or a sequence having at least about 90%, or at least about 95% sequence identity thereto. In some embodiments, the engineered CasX comprises a TSL domain comprising the amino acid sequence of SEQ ID NO: 302, or a sequence having at least about 90%, or at least about 95% sequence identity thereto. In some embodiments, the engineered CasX comprises a RuvC-II domain comprising the amino acid sequence of SEQ ID NO: 303, or a sequence having at least about 90%, or at least about 95% sequence identity thereto. In another particular embodiment, the disclosure provides an engineered CasX having the sequence of SEQ ID NO: 266 (CasX variant 812), or a sequence having at least about 70%, at least about 80%, at least about 90%, or at least about 95%, or at least about 95%, or at least about 96%, or at least about 97%, or at least about 98%, or at least about 99% sequence identity thereto, wherein the engineered CasX exhibits improved specificity compared to CasX variant 515 (SEQ ID NO: 228).

The engineered CasX of the disclosure have one or more improved characteristics compared to a CasX protein from which it was derived; e.g., CasX 515 or the CasX proteins of Table 9 (SEQ ID NOS: 492-500). Exemplary improved characteristics of the engineered CasX embodiments include, but are not limited to improved ability to utilize a greater spectrum of PAM sequences in the editing and/or binding of target nucleic acid, increased nuclease activity, improved editing efficiency, improved editing specificity for the target nucleic acid, decreased off-target editing or cleavage, increased percentage of a eukaryotic genome that can be efficiently edited, increased activity of the nuclease, and improved protein:ERS (RNP) complex stability. In particular, the engineered CasX proteins of the disclosure have an enhanced ability to efficiently edit and/or bind target DNA, when complexed with an ERS as an RNP, utilizing a PAM TC motif, including PAM sequences selected from TTC, ATC, GTC, or CTC, compared to an RNP of a reference CasX protein and a reference gRNA. In the foregoing, the PAM sequence is located at least 1 nucleotide 5′ to the non-target strand of the protospacer having identity with the targeting sequence of the ERS in an assay system compared to the editing efficiency and/or binding of an RNP comprising the reference CasX protein and reference gRNA in a comparable assay system.

Additional engineered CasX of the disclosure include the sequences of SEQ ID NOS: 247-294, as set forth in Table 6, or a sequence having at least about 70%, at least about 80%, at least about 90%, at least about 95%, at least about 98%, at least about 99% sequence identity thereto.

TABLE 6
CasX Protein Sequences

| SEQ ID NO | CasX Protein No. |
|---|---|
| 247 | 793 |
| 248 | 794 |
| 249 | 795 |
| 250 | 796 |
| 251 | 797 |
| 252 | 798 |
| 253 | 799 |
| 254 | 800 |
| 255 | 801 |
| 256 | 802 |
| 257 | 803 |
| 258 | 804 |
| 259 | 805 |
| 260 | 806 |
| 261 | 807 |
| 262 | 808 |
| 263 | 809 |
| 264 | 810 |
| 265 | 811 |
| 266 | 812 |
| 267 | 813 |
| 268 | 814 |
| 269 | 815 |
| 270 | 816 |
| 271 | 817 |
| 272 | 818 |
| 273 | 819 |
| 274 | 820 |
| 275 | 821 |
| 276 | 822 |
| 277 | 823 |
| 278 | 824 |
| 279 | 825 |
| 280 | 826 |
| 281 | 827 |
| 282 | 828 |
| 283 | 829 |
| 284 | 830 |
| 285 | 831 |
| 286 | 832 |
| 287 | 833 |
| 288 | 834 |
| 289 | 835 |
| 290 | 836 |
| 291 | 837 |
| 292 | 838 |
| 293 | 839 |
| 294 | 840 |

c. Engineered CasX Proteins with Domains from Multiple Source Proteins

Also contemplated within the scope of the disclosure are engineered chimeric CasX proteins. As used herein, a “chimeric CasX” protein refers to both a CasX protein containing at least two domains from different sources, as well a CasX protein containing at least one domain that itself is chimeric. Accordingly, in some embodiments, an engineered chimeric CasX protein is one that includes at least two domains isolated or derived from different sources, such as from two different naturally occurring CasX proteins, (e.g., from two different reference CasX proteins), or from two different CasX variant proteins. In the case of split or non-contiguous domains such as helical I, RuvC and OBD, a portion of the non-contiguous domain can be replaced with the corresponding portion from any other source. For example, the helical I-II domain in SEQ ID NO: 2 can be replaced with the corresponding helical I-II sequence from SEQ ID NO: 1, and the like. In some embodiments, the first domain can be selected from the group consisting of the NTSB, TSL, helical I-I, helical I-II, helical II, OBD-I, OBD-II, RuvC-I, and RuvC-II domains. In some embodiments, the second domain is selected from the group consisting of the NTSB, TSL, helical I-I, helical I-II, helical II, OBD-I, OBD-II, RuvC-I, and RuvC-II domains with the second domain being different from the foregoing first domain. Domain sequences from reference CasX proteins, and their coordinates, are shown in Table 4.

In some embodiments, the NTSB domain of the engineered CasX derived from SEQ ID NO: 2 is substituted with the corresponding NTSB sequence from SEQ ID NO: 1, or a sequence having at least about 70%, at least about 80%, at least about 90%, or at least about 95% identity thereto, resulting in a chimeric CasX protein. In some embodiments, the helical I-II domain of the engineered CasX derived from SEQ ID NO: 2 is substituted with the corresponding helical I-II sequence from SEQ ID NO: 1, or a sequence having at least about 70%, at least about 80%, at least about 90%, or at least about 95% identity thereto, resulting in a chimeric CasX protein. In some embodiments, the helical I-II domain and the NTSB domain of the engineered CasX derived from SEQ ID NO: 2 is substituted with the corresponding helical I-II from SEQ ID NO: 1, or a sequence having 1, 2, 3, 4, or 5 mismatches thereto, and the NTSB sequence from SEQ ID NO: 1, or a sequence or a sequence having 1, 2, 3, 4, or 5 mismatches thereto, resulting in a chimeric CasX protein. Exemplary chimeric CasX include, but are not limited to the sequences of SEQ ID NOS: 247-294, 24916-49628, 49746-49747, and 49871-49873, which have the substitution of the NTSB and helical I-II domains from SEQ ID NO: 1, while the other domains are originally derived from SEQ ID NO: 2, where the engineered CasX have additional amino acid changes (i.e., 1, 2, 3, 4, or 5 mismatches) at select locations relative to the domains of the reference CasX.

TABLE 7
CasX 515 domain sequences

| Domain | SEQ ID NO | Amino Acid Sequence |
|---|---|---|
| OBD-I | 295 | QEIKRINKIRRRLVKDSNTKKAGKTGPMKTLLVRVMTPDLRERLENLRKKPENIPQ |
| Helical I-I | 296 | PISNTSRANLNKLLTDYTEMKKAILHVYWEEFQKDPVGLMSRVA |
| NTSB | 297 | QPASKKIDQNKLKPEMDEKGNLTTAGFACSQCGQPLFVYKLEQVSEKGKAYTNYFG |
| | | RCNVAEHEKLILLAQLKPEKDSDEAVTYSLGKFGQ |
| Helical I- | 298 | RALDFYSIHVTKESTHPVKPLAQIAGNRYASGPVGKALSDACMGTIASFLSKYQDI |
| II | | IIEHQKVVKGNQKRLESLRELAGKENLEYPSVTLPPQPHTKEGVDAYNEVIARVRM |
| | | WVNLNLWQKLKLSRDDAKPLLRLKGFPSF |
| Helical II | 299 | PLVERQANEVDWWDMVCNVKKLINEKKEDGKVFWQNLAGYKRQEALRPYLSSEEDR |
| | | KKGKKFARYQLGDLLLHLEKKHGEDWGKVYDEAWERIDKKVEGLSKHIKLEEERRS |
| | | EDAQSKAALTDWLRAKASFVIEGLKEADKDEFCRCELKLOKWYGDLRGKPFAIEAE |
| OBD-II | 300 | NSILDISGFSKQYNCAFIWQKDGVKKLNLYLIINYFKGGKLRFKKIKPEAFEANRF |
| | | YTVINKKSGEIVPMEVNENFDDPNLIILPLAFGKRQGREFIWNDLLSLETGSLKLA |
| | | NGRVIEKTLYNRRTRQDEPALFVALTFERREVLD |
| RuvC-I | 301 | SSNIKPMNLIGVDRGENIPAVIALTDPEGCPLSRFKDSLGNPTHILRIGESYKEKQ |
| | | RTIQAKKEVEQRRAGGYSRKYASKAKNLADDMVRNTARDLLYYAVTQDAMLIFENL |
| | | SRGFGROGKRTFMAERQYTRMEDWLTAKLAYEGLPSKTYLSKTLAQYTSKTC |
| TSL | 302 | SNCGFTITSADYDRVLEKLKKTATGWMTTINGKELKVEGQITYYNRYKRQNVVKDL |
| | | SVELDRLSEESVNNDISSWTKGRSGEALSLLKKRFSHRPVQEKFVCLNCGFETH |
| RuvC-II | 303 | ADEQAALNIARSWLFLRSQEYKKYQTNKTTGNTDKRAFVETWQSFYRKKLKEVWKP |
| | | AV |

d. Protein Affinity for the ERS

In some embodiments, an engineered CasX protein has improved affinity for the ERS relative to a CasX protein from which it was derived, leading to the formation of the ribonucleoprotein complex. Without wishing to be bound by theory, in some embodiments amino acid changes in the helical I domain can increase the binding affinity of the engineered CasX protein with the ERS sequence, while changes in the helical II domain can increase the binding affinity of the engineered CasX protein with the guide scaffold stem loop, and changes in the oligonucleotide binding domain (OBD) increase the binding affinity of the engineered CasX protein with the ERS triplex. Increased affinity of the engineered CasX protein for the ERS may, for example, result in a lower K_{d} for the generation of an RNP complex, which can, in some cases, result in a more stable RNP complex formation. In some embodiments, increased affinity of the engineered CasX protein for the ERS results in increased stability of the RNP complex when delivered to human cells. This increased stability can affect the function and utility of the complex in the cells of a subject, as well as result in improved pharmacokinetic properties in blood, when delivered to a subject. In some embodiments, increased affinity of the engineered CasX protein, and the resulting increased stability of the RNP complex, allows for a lower dose of the engineered CasX protein to be delivered to the subject or cells while still having the desired activity, for example in vivo or in vitro gene editing. In some embodiments, a higher affinity (tighter binding) of an engineered CasX protein to an ERS allows for a greater amount of editing events when both the engineered CasX protein and the ERS remain in an RNP complex. Increased editing events can be assessed using editing assays described herein. In some embodiments, the K_{d} of an engineered CasX protein for an ERS is increased relative to a parental CasX protein mutagenized to create the engineered CasX. In some embodiments, the K_{d} of an engineered CasX for an ERS is increased relative to the CasX from which it was derived by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100. In some embodiments, the engineered CasX has about 1.1 to about 100-fold increased binding affinity to the ERS relative to the CasX from which it was derived; e.g., CasX 515.

In some embodiments, increased affinity of the engineered CasX protein for the ERS results in increased stability of the ribonucleoprotein complex when delivered to mammalian cells, including in vivo delivery to a subject. This increased stability can affect the function and utility of the complex in the cells of a subject, as well as result in improved pharmacokinetic properties in blood, when delivered to a subject. In some embodiments, increased affinity of the engineered CasX protein, and the resulting increased stability of the ribonucleoprotein complex, allows for a lower dose of the engineered CasX protein to be delivered to the subject or cells while still having the desired activity; for example in vivo or in vitro gene editing. The increased ability to form RNP and keep them in stable form can be assessed using assays such as the in vitro cleavage assays described in the Examples herein. In some embodiments, RNP comprising the engineered CasX of the disclosure are able to achieve a k_{cleave} rate when complexed as an RNP that is at last 2-fold, at least 5-fold, or at least 10-fold higher compared to RNP comprising a CasX from which it was derived; e.g., CasX 515.

Methods of measuring engineered CasX protein binding affinity for an ERS and determination of the cleavage competent fractions include in vitro methods using purified engineered CasX protein and ERS, as described in the Examples. The binding affinity for engineered CasX proteins can be measured by fluorescence polarization if the ERS or engineered CasX protein is tagged with a fluorophore. Alternatively, or in addition, binding affinity can be measured by biolayer interferometry, electrophoretic mobility shift assays (EMSAs), or filter binding. Additional standard techniques to quantify absolute affinities of RNA binding proteins such as the engineered CasX of the disclosure for specific ERS include, but are not limited to, isothermal calorimetry (ITC), and surface plasmon resonance (SPR), as well as the methods of the Examples.

e. Affinity for Target Nucleic Acid

In some embodiments, an engineered CasX protein has increased binding affinity for a target nucleic acid relative to the affinity of a CasX protein from which it was derived for a target nucleic acid. Engineered CasX with higher affinity for their target nucleic acid may, in some embodiments, cleave the target nucleic acid sequence more rapidly than a reference CasX protein that does not have increased affinity for the target nucleic acid.

In some embodiments, the improved affinity for the target nucleic acid comprises improved affinity for the target sequence or protospacer sequence of the target nucleic acid, improved affinity for the PAM sequence, an improved ability to search DNA for the target sequence, or any combinations thereof. Without wishing to be bound by theory, it is thought that CRISPR/Cas system proteins such as CasX may find their target sequences by one-dimension diffusion along a DNA molecule. The process is thought to include (1) binding of the ribonucleoprotein to the DNA molecule followed by (2) stalling at the target sequence, either of which may be, in some embodiments, affected by improved affinity of engineered CasX proteins for a target nucleic acid sequence, thereby improving function of the engineered CasX protein.

Without wishing to be bound by theory, it is possible that amino acid changes in the NTSB domain that increase the efficiency of unwinding, or capture, of a non-target nucleic acid strand in the unwound state, can increase the affinity of engineered CasX proteins for target nucleic acid. Alternatively, or in addition, amino acid changes in the NTSB domain that increase the ability of the NTSB domain to stabilize DNA during unwinding can increase the affinity of engineered CasX proteins for target nucleic acid. Alternatively, or in addition, amino acid changes in the OBD may increase the affinity of engineered CasX protein binding to the protospacer adjacent motif (PAM), thereby increasing affinity of the engineered CasX protein for target nucleic acid. Alternatively, or in addition, amino acid changes in the Helical I and/or II, RuvC and TSL domains that increase the affinity of the engineered CasX protein for the target nucleic acid strand can increase the affinity of the engineered CasX protein for target nucleic acid.

In some embodiments, binding affinity of an engineered CasX protein of the disclosure for a target nucleic acid molecule is increased relative to a CasX protein from which it was derived. In some embodiments, the engineered CasX protein has increased binding affinity to the target nucleic acid compared to the CasX 515 variant by a factor of at least about 1.1, at least about 1.2, at least about 1.3, at least about 1.4, at least about 1.5, at least about 1.6, at least about 1.7, at least about 1.8, at least about 1.9, at least about 2, at least about 3, at least about 4, at least about 5, at least about 6, at least about 7, at least about 8, at least about 9, at least about 10, at least about 15, at least about 20, at least about 25, at least about 30, at least about 35, at least about 40, at least about 45, at least about 50, at least about 60, at least about 70, at least about 80, at least about 90, or at least about 100-fold greater.

Methods of measuring CasX protein affinity for a target and/or non-target nucleic acid molecule may include electrophoretic mobility shift assays (EMSAs), filter binding, isothermal calorimetry (ITC), and surface plasmon resonance (SPR), fluorescence polarization and biolayer interferometry (BLI). Further methods of measuring CasX protein affinity for a target include the in vitro biochemical assays of the Examples that measure DNA cleavage events over time.

In some embodiments, an engineered CasX protein with improved target nucleic acid affinity has increased affinity for or the ability to utilize specific PAM sequences other than the canonical TTC PAM recognized by the reference CasX protein of SEQ ID NO: 2, including PAM sequences selected from the group consisting of ATC, GTC, and CTC, thereby increasing the amount of target nucleic acid that can be edited compared to wild-type CasX nucleases or to CasX variants 491 or 515. Without wishing to be bound by theory, it is possible that these engineered CasX may interact more strongly with DNA overall and may have an increased ability to access and edit sequences within the target nucleic acid due to the ability to more strongly bind or utilize PAM sequences beyond those of wild-type reference CasX or the nucleases of CasX 491 or 515, thereby allowing for a more efficient search process of the CasX protein for the target sequence. A higher overall affinity for DNA also, in some embodiments, can increase the frequency at which a CasX protein can effectively start and finish a binding and unwinding step, thereby facilitating target strand invasion and R-loop formation, and ultimately the cleavage of a target nucleic acid sequence.

f. Improved Specificity for a Target Site

In some embodiments, an engineered CasX protein has improved specificity for a target nucleic acid sequence relative to a CasX protein from which it was derived. As used herein, “specificity,” sometimes referred to as “target specificity,” refers to the degree to which a CRISPR/Cas system ribonucleoprotein complex cleaves off-target sequences that are similar, but not identical to the target nucleic acid sequence; e.g., an engineered CasX RNP with a higher degree of specificity would exhibit reduced off-target effects, or cleavage of sequences relative to a CasX protein from which it was derived. Without wishing to be bound by theory, it is possible that amino acid changes in the helical I and II domains that increase the specificity of the engineered CasX protein for the target nucleic acid strand can increase the specificity of the engineered CasX protein for the target nucleic acid overall. In some embodiments, amino acid changes that increase specificity of engineered CasX proteins for target nucleic acid may also result in decreased affinity of engineered CasX proteins for DNA.

The specificity, and the reduction of potentially deleterious off-target effects, of CRISPR/Cas system proteins can be vitally important in order to achieve an acceptable therapeutic index for use in mammalian subjects. As used herein, “off-target effects” refers to off-target effects of unintended cleavage and mutations at untargeted genomic sites showing a similar but not an identical sequence compared to the target site. In some embodiments, the off-target effects exhibited by the engineered CasX complexed with an ERS and linked targeting sequence is less than about 5%, less than about 4%, less than 3%, less than about 2%, less than about 1%, less than about 0.5%, less than 0.1% in cells. In some embodiments the off-target effects are determined in silico. In some embodiments the off-target effects are determined in an in vitro cell-free assay. In some embodiments the off-target effects are determined in a cell-based assay. In some embodiments, the engineered CasX protein comprising a pair of mutations as depicted in Table 22, or further variations thereof, and demonstrates increased on-target editing activity, increased specificity (or decreased off-target activity), increased specificity ratio, or a combination thereof relative to SEQ ID NO: 228 (CasX variant 515).

Methods of testing CasX protein (such as engineered or reference CasX) target specificity may include guide and Circularization for In vitro Reporting of Cleavage Effects by Sequencing (CIRCLE-seq), or similar methods. In brief, in CIRCLE-seq techniques, genomic DNA is sheared and circularized by ligation of stem-loop adapters, which are nicked in the stem-loop regions to expose 4 nucleotide palindromic overhangs. This is followed by intramolecular ligation and degradation of remaining linear DNA. Circular DNA molecules containing a CasX cleavage site are subsequently linearized with CasX, and adapter adapters are ligated to the exposed ends followed by high-throughput sequencing to generate paired end reads that contain information about the off-target site. Additional assays that can be used to detect off-target events, and therefore CasX protein specificity include assays used to detect and quantify indels (insertions and deletions) formed at those selected off-target sites such as mismatch-detection nuclease assays and next generation sequencing (NGS). Exemplary mismatch-detection assays include nuclease assays, in which genomic DNA from cells treated with CasX and ERS is PCR amplified, denatured and rehybridized to form hetero-duplex DNA, containing one wild-type strand and one strand with an indel. Mismatches are recognized and cleaved by mismatch detection nucleases, such as Surveyor nuclease or T7 endonuclease I. Methods to evaluate the specificity of the engineered CasX, along with supporting data demonstrating improved specificity of embodiments of engineered CasX, are described in the Examples.

g. Protospacer and PAM Sequences

Herein, the protospacer is defined as the DNA sequence complementary to the targeting sequence of the guide RNA and the DNA complementary to that sequence, referred to as the target strand and non-target strand, respectively. As used herein, the PAM is a nucleotide sequence proximal to the protospacer that, in conjunction with the targeting sequence of the guide RNA, helps the orientation and positioning of the CasX for the potential cleavage of the protospacer strand(s).

PAM sequences may be degenerate, and specific RNP constructs may have different preferred and tolerated PAM sequences that support different efficiencies of cleavage. Following convention, unless stated otherwise, the disclosure refers to both the PAM and the protospacer sequence and their directionality according to the orientation of the non-target strand. This does not imply that the PAM sequence of the non-target strand, rather than the target strand, is determinative of cleavage or mechanistically involved in target recognition. For example, when reference is to a TTC PAM, it may in fact be the complementary GAA sequence that is required for target cleavage, or it may be some combination of nucleotides from both strands. In the case of the CasX proteins disclosed herein, the PAM is located 5′ of the protospacer with a single nucleotide separating the PAM from the first nucleotide of the protospacer. Thus, in the case of reference CasX, a TTC PAM should be understood to mean a sequence following the formula 5′- . . . NNTTCN(protospacer)NNNNNN . . . 3′ (SEQ ID NO: 304) where ‘N’ is any DNA nucleotide and ‘(protospacer)’ is a DNA sequence having identity with the targeting sequence of the guide RNA. In the case of an engineered CasX with expanded PAM recognition, a TTC, CTC, GTC, or ATC PAM should be understood to mean a sequence following the formulae: 5′- . . . NNTTCN(protospacer)NNNNNN . . . 3′ (SEQ ID NO: 304); 5′- . . . NNCTCN(protospacer)NNNNNN . . . 3′ (SEQ ID NO: 305); 5′- . . . NNGTCN(protospacer)NNNNNN . . . 3′ (SEQ ID NO: 306); or 5′- . . . NNATCN(protospacer)NNNNNN . . . 3′ (SEQ ID NO: 307). Alternatively, a TC PAM should be understood to mean a sequence following the formula 5′- . . . NNNTCN(protospacer)NNNNNN . . . 3′ (SEQ ID NO: 308).

In some embodiments, the engineered CasX proteins of the disclosure have an improved ability to efficiently edit and/or bind target nucleic acid, when complexed with an ERS as an RNP, utilizing a PAM TC motif, including PAM sequences selected from TTC, ATC, GTC, or CTC, (in a 5′ to 3′ orientation), compared to an RNP of an RNP of a CasX protein from which it was derived, such as CasX 515 complexed with gRNA 174. In the foregoing, the PAM sequence is located at least 1 nucleotide 5′ to the non-target strand of the protospacer having identity with the targeting sequence of the ERS in an assay system. In one embodiment, an RNP of an engineered CasX and ERS exhibits greater editing and/or binding of a target sequence in the target nucleic acid compared to an RNP of a CasX protein from which it was derived, such as CasX 515, and gRNA 174 in a comparable assay system, wherein the PAM sequence of the target DNA is TTC. In another embodiment, an RNP of an engineered CasX and ERS exhibits greater editing and/or binding of a target sequence in the target nucleic acid compared to an RNP comprising an RNP of a CasX protein from which it was derived, such as CasX 515 and gRNA 174 in a comparable assay system, wherein the PAM sequence of the target DNA is ATC. In another embodiment, an RNP of an engineered CasX and ERS exhibits greater editing and/or binding of a target sequence in the target nucleic acid compared to an RNP comprising an RNP of a CasX protein from which it was derived, such as CasX 515, and gRNA 174 in a comparable assay system, wherein the PAM sequence of the target DNA is CTC. In another embodiment, an RNP of an engineered CasX and ERS exhibits greater editing and/or binding of a target sequence in the target nucleic acid compared to an RNP comprising a an RNP of a CasX protein from which it was derived and gRNA 174 in a comparable assay system, wherein the PAM sequence of the target DNA is GTC. In the foregoing embodiments, the increased editing and/or binding affinity for the one or more PAM sequences is at least about 1.5-fold, at least about 2-fold, at least about 4-fold, at least about 10-fold, at least about 20-fold, at least about 30-fold, or at least about 40-fold greater or more compared to the editing and/or binding affinity of an RNP of a CasX protein from which it was derived and gRNA 174 for the PAM sequences.

h. Catalytic Activity

The ribonucleoprotein complex of the eCasX:ERS systems disclosed herein comprise an engineered CasX complexed with an ERS that binds to a target nucleic acid and cleaves the target nucleic acid. In some embodiments, an engineered CasX protein has improved catalytic activity relative to a CasX protein from which it was derived. Without wishing to be bound by theory, it is thought that in some cases cleavage of the target strand can be a limiting factor for Cas12-like molecules in creating a dsDNA break. In some embodiments, engineered CasX proteins improve bending of the target strand of DNA and cleavage of this strand, resulting in an improvement in the overall efficiency of dsDNA cleavage by the CasX ribonucleoprotein complex.

Engineered CasX with increased double-strand nuclease activity can be generated, for example, through amino acid changes in the RuvC nuclease domain. In the foregoing, the engineered CasX generates a double-stranded break within 18-26 nucleotides 5′ of a PAM site on the target strand and 10-18 nucleotides 3′ on the non-target strand. Nuclease activity can be assayed by a variety of methods, including those of the Examples. In some embodiments, an engineered CasX has a k_{cleave} constant that is improved at least about 10%, at least about 20%, at least about 30%, at least about 40%, or at least about 50% or more compared to a CasX protein from which it was derived.

In some embodiments, an engineered CasX protein has the improved characteristic of forming RNP with ERS that result in a higher percentage of cleavage-competent RNP compared to an RNP of a CasX protein from which it was derived and the gRNA variant. By cleavage competent, it is meant that the RNP that is formed has the ability to cleave the target nucleic acid. In some embodiments, the RNP of the engineered CasX and the ERS exhibit at least a 2-fold, or at least a 3-fold, or at least a 4-fold, or at least a 5-fold, or at least a 10-fold cleavage rate compared to an RNP of a CasX protein from which it was derived. In the foregoing embodiment, the improved competency rate can be demonstrated in an in vitro assay, such as described in the Examples.

In some embodiments, the disclosure provides engineered CasX proteins that are catalytically dead but retains the ability to bind a target nucleic acid. An exemplary catalytically dead engineered CasX protein comprises one or more mutations in the active site of the RuvC domain of the CasX protein. In some embodiments, a catalytically dead engineered CasX protein comprises substitutions at residues 672, 769 and/or 935 relative to the sequence of SEQ ID NO: 1. In one embodiment, a catalytically dead engineered CasX protein comprises substitutions of D672A, E769A and/or D935A relative to the reference CasX protein of SEQ ID NO: 1. In other embodiments, a catalytically dead engineered CasX protein comprises substitutions at amino acids 659, 756 and/or 922 relative to the reference CasX protein of SEQ ID NO: 2. In some embodiments, a catalytically dead engineered CasX protein comprises D659A, E756A and/or D922A substitutions relative to the reference CasX protein of SEQ ID NO: 2. In some embodiments, the disclosure provides a catalytically-dead engineered CasX of any one of SEQ ID NOS: 156, 739-907, 739-907, 11568-22227, 23572-24915, and 49719-49735 comprising the foregoing mutations to render them catalytically dead.

i. Engineered CasX Fusion Proteins

In some embodiments, the disclosure provides engineered CasX proteins comprising a heterologous protein fused to the CasX, including the engineered CasX of any of the embodiments described herein. This includes engineered CasX comprising N-terminal, C-terminal, or internal fusions of the CasX to a heterologous protein or domain thereof.

In some embodiments, the engineered CasX fusion protein comprises any one of the sequences of SEQ ID NOS: 247-294, 24916-49628, 49746-49747, or 49871-49873 fused to one or more proteins or domains thereof that have a different activity of interest or impart a different functional property, resulting in a fusion protein.

A variety of heterologous polypeptides are suitable for inclusion in an engineered CasX fusion protein of the disclosure. In some cases, the fusion partner can modulate transcription (e.g., inhibit transcription, increase transcription) of a target nucleic acid. For example, in some cases the fusion partner is a protein (or a domain from a protein) that inhibits transcription (e.g., a transcriptional repressor, a protein that functions via recruitment of transcription inhibitor proteins, modification of target nucleic acid such as methylation, recruitment of a DNA modifier, modulation of histones associated with target nucleic acid, recruitment of a histone modifier such as those that modify acetylation and/or methylation of histones, and the like). In some cases, the fusion partner is a protein (or a domain from a protein) that increases transcription (e.g., a transcription activator, a protein that acts via recruitment of transcription activator proteins, modification of target nucleic acid such as demethylation, recruitment of a DNA modifier, modulation of histones associated with target nucleic acid, recruitment of a histone modifier such as those that modify acetylation and/or methylation of histones, and the like).

In some cases, a fusion partner has enzymatic activity that modifies a target nucleic acid sequence; e.g., nuclease activity, methyltransferase activity, demethylase activity, DNA repair activity, DNA damage activity, deamination activity, dismutase activity, alkylation activity, depurination activity, oxidation activity, pyrimidine dimer forming activity, integrase activity, transposase activity, recombinase activity, polymerase activity, ligase activity, helicase activity, photolyase activity or glycosylase activity.

Examples of proteins (or fragments thereof) that can be used as a fusion partner to decrease transcription include but are not limited to: transcriptional repressors such as the Kruppel associated box (KRAB or SKD); KOX1 repression domain; the Mad mSIN3 interaction domain (SID); the ERF repressor domain (ERD), the SRDX repression domain (e.g., for repression in plants), and the like; histone lysine methyltransferases such as Pr-SET7/8, SUV4-20H1, RIZI, and the like; histone lysine demethylases such as JMJD2A/JHDM3A, JMJD2B, JMJD2C/GASC1, JMJD2D, JARID1A/RBP2, JARID1B/PLU-1, JARID 1C/SMCX, JARID1D/SMCY, and the like; histone lysine deacetylases such as HDAC1, HDAC2, HDAC3, HDAC8, HDAC4, HDAC5, HDAC7, HDAC9, SIRT1, SIRT2, HDAC11, and the like; DNA methylases such as HhaI DNA m5c-methyltransferase (M.HhaI), DNA methyltransferase 1 (DNMT1), DNA methyltransferase 3 alpha (DNMT3A) and subdomains such as DNMT3A catalytic domain and ATRX-DNMT3-DNMT3L domain (ADD), DNMT3L interaction domain (DNMT3L), DNA methyltransferase 3 beta (DNMT3B), Friend of GATA-1 (FOG), METI, DRM3 (plants), ZMET2, CMT1, CMT2 (plants), and the like; and periphery recruitment elements such as Lamin A, Lamin B, and the like.

In some cases, the fusion partner to an engineered CasX has enzymatic activity that modifies the target nucleic acid (e.g., ssRNA, dsRNA, ssDNA, dsDNA). Examples of enzymatic activity that can be provided by the fusion partner include but are not limited to: nuclease activity such as that provided by a restriction enzyme (e.g., FokI nuclease), methyltransferase activity such as that provided by a methyltransferase (e.g., Hhal DNA m5c-methyltransferase (M.Hhal), DNA methyltransferase 1 (DNMT1), DNA methyltransferase 3 alpha (DNMT3A) and subdomains such as DNMT3A catalytic domain and ATRX-DNMT3-DNMT3L domain (ADD), DNMT3L interaction domain (DNMT3L), DNA methyltransferase 3 beta (DNMT3B), METI, DRM3 (plants), ZMET2, CMT1, CMT2 (plants), and the like); demethylase activity such as that provided by a demethylase (e.g., Ten-Eleven Translocation (TET) dioxygenase 1 (TET 1 CD), TET1, DME, DML1, DML2, ROS1, and the like), DNA repair activity, DNA damage activity, deamination activity such as that provided by a deaminase (e.g., a cytosine deaminase enzyme, e.g., an APOBEC protein such as rat apolipoprotein B mRNA editing enzyme, catalytic polypeptide 1 {APOBEC1}), dismutase activity, alkylation activity, depurination activity, oxidation activity, pyrimidine dimer forming activity, integrase activity such as that provided by an integrase and/or resolvase (e.g., Gin invertase such as the hyperactive mutant of the Gin invertase, GinH106Y; human immunodeficiency virus type 1 integrase (IN); Tn3 resolvase; and the like), transposase activity, recombinase activity such as that provided by a recombinase (e.g., catalytic domain of Gin recombinase), polymerase activity, ligase activity, helicase activity, photolyase activity, and glycosylase activity).

In some cases, an engineered CasX protein of the present disclosure is fused to a polypeptide selected from a domain for increasing transcription (e.g., a VP16 domain, a VP64 domain), a domain for decreasing transcription (e.g., a KRAB domain, e.g., from the Kox1 protein), a core catalytic domain of a histone acetyltransferase (e.g., histone acetyltransferase p300), a protein/domain that provides a detectable signal (e.g., a fluorescent protein such as GFP), a nuclease domain (e.g., a Fokl nuclease), or a base editor (e.g., cytidine deaminase such as APOBEC1).

In some cases, an engineered CasX protein of the present disclosure can include an endosomal escape peptide. In some cases, an endosomal escape polypeptide comprises the amino acid sequence GLFXALLXLLXSLWXLLLXA (SEQ ID NO: 309), wherein each X is independently selected from lysine, histidine, and arginine. In some cases, an endosomal escape polypeptide comprises the amino acid sequence GLFHALLHLLHSLWHLLLHA (SEQ ID NO: 310), or HHHHHHHHH (SEQ ID NO: 311). In some embodiments, an engineered CasX comprises a sequence of any one of the sequences of SEQ ID NOS: 247-294, 24916-49628, 49746-49747, or 49871-49873 and an endosomal escape polypeptide.

Additionally or alternatively, an engineered CasX protein of the present disclosure may be fused to a polypeptide permeant domain to promote uptake by the cell. A number of permeant domains are known in the art and may be used in the non-integrating polypeptides of the present disclosure, including peptides, peptidomimetics, and non-peptide carriers. For example, WO2017/106569 and US20180363009A1, incorporated by reference herein in its entirety, describe fusion of a Cas protein with one or more nuclear localization sequences (NLS) to facilitate cell uptake. In other embodiments, a permeant peptide may be derived from the third alpha helix of Drosophila melanogaster transcription factor Antennapaedia, referred to as penetratin, which comprises the amino acid sequence RQIKIWFQNRRMKWKK (SEQ ID NO: 312). As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 Rev protein, nona-arginine, octa-arginine, and the like. The site at which the fusion is made may be selected in order to optimize the biological activity, secretion or binding characteristics of the polypeptide. The optimal site will be determined by routine experimentation.

In some embodiments, a heterologous polypeptide (a fusion partner) for use with an engineered CasX provides for subcellular localization; i.e., the heterologous polypeptide contains a subcellular localization sequence (e.g., a nuclear localization signal (NLS) for targeting to the nucleus, a sequence to keep the fusion protein out of the nucleus, e.g., a nuclear export sequence (NES), a sequence to keep the fusion protein retained in the cytoplasm, a mitochondrial localization signal for targeting to the mitochondria, a chloroplast localization signal for targeting to a chloroplast, an ER retention signal, and the like). In some embodiments, a subject RNA-guided polypeptide or a conditionally active RNA-guided polypeptide and/or subject CasX fusion protein does not include a NLS so that the protein is not targeted to the nucleus, which can be advantageous; e.g., when the target nucleic acid is an RNA that is present in the cytosol. In some embodiments, a fusion partner can provide a tag (i.e., the heterologous polypeptide is a detectable label) for ease of tracking and/or purification (e.g., a fluorescent protein, e.g., green fluorescent protein (GFP), yellow fluorescent protein (YFP), red fluorescent protein (RFP), cyan fluorescent protein (CFP), mCherry, tdTomato, and the like; a histidine tag, e.g., a 6×His tag; a hemagglutinin (HA) tag; a FLAG tag; a Myc tag; and the like).

In some cases, an engineered CasX protein includes (is fused to) a nuclear localization signal (NLS). Non-limiting examples of NLSs suitable for use with an engineered CasX include sequences having at least about 80%, at least about 90%, or at least about 95% identity or are identical to sequences derived from: the NLS of the SV40 virus large T-antigen, having the amino acid sequence PKKKRKV (SEQ ID NO: 313); the NLS from nucleoplasmin (e.g., the nucleoplasmin bipartite NLS with the sequence KRPAATKKAGQAKKKK (SEQ ID NO: 314); the c-myc NLS having the amino acid sequence PAAKRVKLD (SEQ ID NO: 315)) or RQRRNELKRSP (SEQ ID NO: 316); the hRNPA1 M9 NLS having the sequence NQSSNFGPMKGGNFGGRSSGPYGGGGQYFAKPRNQGGY (SEQ ID NO: 317); the sequence RMRIZFKNKGKDTAELRRRRVEVSVELRKAKKDEQILKRRNV (SEQ ID NO: 318) of the IBB domain from importin-alpha; the sequences VSRKRPRP (SEQ ID NO: 319) and PPKKARED (SEQ ID NO: 320) of the myoma T protein; the sequence PQPKKKPL (SEQ ID NO: 321) of human p53; the sequence SALIKKKKKMAP (SEQ ID NO: 322) of mouse c-abl IV; the sequences DRLRR (SEQ ID NO: 323) and PKQKKRK (SEQ ID NO: 324) of the influenza virus NS1; the sequence RKLKKKIKKL (SEQ ID NO: 325) of the Hepatitis virus delta antigen; the sequence REKKKFLKRR (SEQ ID NO: 326) of the mouse Mxl protein; the sequence KRKGDEVDGVDEVAKKKSKK (SEQ ID NO: 327) of the human poly(ADP-ribose) polymerase; the sequence RKCLQAGMNLEARKTKK (SEQ ID NO: 328) of the steroid hormone receptors (human) glucocorticoid; the sequence PRPRKIPR (SEQ ID NO: 329) of Borna disease virus P protein (BDV-P1); the sequence PPRKKRTVV (SEQ ID NO: 330) of hepatitis C virus nonstructural protein (HCV-NS5A); the sequence NLSKKKKRKREK (SEQ ID NO: 331) of LEF1; the sequence RRPSRPFRKP (SEQ ID NO: 332) of ORF57 simirae; the sequence KRPRSPSS (SEQ ID NO: 333) of EBV LANA; the sequence KRGINDRNFWRGENERKTR (SEQ ID NO: 334) of Influenza A protein; the sequence PRPPKMARYDN (SEQ ID NO: 335) of human RNA helicase A (RHA); the sequence KRSFSKAF (SEQ ID NO: 336) of nucleolar RNA helicase II; the sequence KLKIKRPVK (SEQ ID NO: 337) of TUS-protein; the sequence PKKKRKVPPPPAAKRVKLD (SEQ ID NO: 338) associated with importin-alpha; the sequence PKTRRRPRRSQRKRPPT (SEQ ID NO: 339) from the Rex protein in HTLV-1; the sequence SRRRKANPTKLSENAKKLAKEVEN (SEQ ID NO: 340) from the EGL-13 protein of Caenorhabditis elegans; and the sequences KTRRRPRRSQRKRPPT (SEQ ID NO: 341), RRKKRRPRRKKRR (SEQ ID NO: 342), PKKKSRKPKKKSRK (SEQ ID NO: 343), HKKKHPDASVNFSEFSK (SEQ ID NO: 344), QRPGPYDRPQRPGPYDRP (SEQ ID NO: 345), LSPSLSPLLSPSLSPL (SEQ ID NO: 346), RGKGGKGLGKGGAKRHRK (SEQ ID NO: 347), PKRGRGRPKRGRGR (SEQ ID NO: 348), PKKKRKVPPPPAAKRVKLD (SEQ ID NO: 349), PKKKRKVPPPPKKKRKV (SEQ ID NO: 350), PAKRARRGYKC (SEQ ID NO: 351), KLGPRKATGRW (SEQ ID NO: 352), PRRKREE (SEQ ID NO: 353), PYRGRKE (SEQ ID NO: 354), PLRKRPRR (SEQ ID NO: 355), PLRKRPRRGSPLRKRPRR (SEQ ID NO: 356), PAAKRVKLDGGKRTADGSEFESPKKKRKV (SEQ ID NO: 357), PAAKRVKLDGGKRTADGSEFESPKKKRKVGIHGVPAA (SEQ ID NO: 358), PAAKRVKLDGGKRTADGSEFESPKKKRKVAEAAAKEAAAKEAAAKA (SEQ ID NO: 359), PAAKRVKLDGGKRTADGSEFESPKKKRKVPG (SEQ ID NO: 360), KRKGSPERGERKRHW (SEQ ID NO: 361), KRTADSQHSTPPKTKRKVEFEPKKKRKV (SEQ ID NO: 362), and PKKKRKVGGSKRTADSQHSTPPKTKRKVEFEPKKKRKV (SEQ ID NO: 363). In general, NLS (or multiple NLSs) are of sufficient strength to drive accumulation of an engineered CasX fusion protein in the nucleus of a eukaryotic cell. Detection of accumulation in the nucleus may be performed by any suitable technique. For example, a detectable marker may be fused to an engineered CasX fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

The disclosure contemplates assembly of multiple NLS in various configurations for linkage to the engineered CasX proteins of the embodiments. In some embodiments, one or more NLS are linked at or near the N-terminus of the engineered CasX protein. In other embodiments, one or more NLS are linked at or near the C-terminus of the engineered CasX protein. In other embodiments, one or more NLS are linked at or near both the N- and C-terminus of the engineered CasX protein. In some embodiments, the NLS linked to the N-terminus of the engineered CasX protein are identical to the NLS linked to the C-terminus. In some embodiments, the NLS linked to the N-terminus of the engineered CasX protein are different from the NLS linked to the C-terminus. In some embodiments, the NLS can be linked within 1, 2, 3, 4, 5, 6, 7, 8, 9 or 10 amino acids to the N- or C-terminus of the engineered CasX protein. In some embodiments, the NLS can be linked to the N- or C-terminus of the engineered CasX protein by a linker peptide, embodiments of which are described herein. In some embodiments, an NLS is linked to another NLS by a linker. In other embodiments, the NLS linked to the N-terminus of the engineered CasX protein are different to the NLS linked to the C-terminus. In some embodiments, the NLS linked to the N-terminus of the engineered CasX protein are selected from the group consisting of the N-terminal sequences as set forth in Table 8 (SEQ ID NOS: 364-410). In some embodiments, the NLS linked to the C-terminus of the engineered CasX protein are selected from the group consisting of the C-terminal sequences as set forth in Table 8 (SEQ ID NOS: 411-457).

Detection of accumulation in the nucleus of the engineered CasX fusion proteins may be performed by any suitable technique. For example, a detectable marker may be fused to an engineered CasX fusion protein such that location within a cell may be visualized. Cell nuclei may also be isolated from cells, the contents of which may then be analyzed by any suitable process for detecting protein, such as immunohistochemistry, Western blot, or enzyme activity assay. Accumulation in the nucleus may also be determined indirectly.

TABLE 8
NLS Sequences

| | SEQ ID | | SEQ ID |
|---|---|---|---|
| N-terminal Sequences | NO | C-terminal Sequences | NO |
| PKKKRKVGGSPKKKRKVSRQEIKRINKIRR | 364 | TLESPAAKRVKLDGGSPAAKRVKLDGG | 411 |
| RLVKDSNTKKAGKTGP | | SPAAKRVKLDGGSPAAKRVKLDGGSPA | |
| | | AKRVKLDGGSPAAKRVKLDTLESKRPA | |
| | | ATKKAGQAKKKKGGSKRPAATKKAGQA | |
| | | KKKKGGSKRPAATKKAGQAKKKKGGSK | |
| | | RPAATKKAGQAKKKK | |
| PKKKRKVGGSPKKKRKVGGSPKKKRKVGGS | 365 | TLESKRPAATKKAGQAKKKKTLESKRP | 412 |
| PKKKRKVSRQEIKRINKIRRRLVKDSNTKK | | AATKKAGQAKKKKGGSKRPAATKKAGQ | |
| AGKTGP | | AKKKKGGSKRPAATKKAGQAKKKKGGS | |
| | | KRPAATKKAGQAKKKKGGSKRPAATKK | |
| | | AGQAKKKKGGSKRPAATKKAGQAKKKK | |
| PKKKRKVGGSPKKKRKVGGSPKKKRKVGGS | 366 | TLESKRPAATKKAGQAKKKKGGSKRPA | 413 |
| PKKKRKVGGSPKKKRKVGGSPKKKRKVSRQ | | ATKKAGQAKKKKTLESPKKKRKVGGSP | |
| EIKRINKIRRRLVKDSNTKKAGKTGP | | KKKRKVGGSPKKKRKVGGSPKKKRKV | |
| PAAKRVKLDGGSPAAKRVKLDSRQEIKRIN | 367 | TLEGGSPKKKRKVTLESPKKKRKVGGS | 414 |
| KIRRRLVKDSNTKKAGKTGP | | PKKKRKVGGSPKKKRKVGGSPKKKRKV | |
| PAAKRVKLDGGSPAAKRVKLDGGSPAAKRV | 368 | TLEGGSPKKKRKVTLESPAAKRVKLDG | 415 |
| KLDGGSPAAKRVKLDSRQEIKRINKIRRRL | | GSPAAKRVKLDGGSPAAKRVKLDGGSP | |
| VKDSNTKKAGKTGP | | AAKRVKLD | |
| PAAKRVKLDGGSPAAKRVKLDGGSPAAKRV | 369 | TLEGGSPKKKRKVTLESPAAKRVKLDG | 416 |
| KLDGGSPAAKRVKLDGGSPAAKRVKLDGGS | | GSPAAKRVKLDGGSPAAKRVKLDGGSP | |
| PAAKRVKLDSRQEIKRINKIRRRLVKDSNT | | AAKRVKLDGGSPAAKRVKLDGGSPAAK | |
| KKAGKTGP | | RVKLD | |
| KRPAATKKAGQAKKKKSRDISRQEIKRINK | 370 | TLEGGSPKKKRKVTLESKRPAATKKAG | 417 |
| IRRRLVKDSNTKKAGKTGP | | QAKKKK | |
| KRPAATKKAGQAKKKKSRQEIKRINKIRRR | 371 | TLEGGSPKKKRKVTLESKRPAATKKAG | 418 |
| LVKDSNTKKAGKTGP | | QAKKKKGGSKRPAATKKAGQAKKKK | |
| KRPAATKKAGQAKKKKGGSKRPAATKKAGQ | 372 | TLEGGSPKKKRKVTLEGGSPKKKRKV | 419 |
| AKKKKSRDISRQEIKRINKIRRRLVKDSNT | | | |
| KKAGKTGP | | | |
| KRPAATKKAGQAKKKKGGSKRPAATKKAGQ | 373 | TLEGGSPKKKRKVTLEGGSPKKKRKV | 420 |
| AKKKKGGSKRPAATKKAGQAKKKKGGSKRP | | | |
| AATKKAGQAKKKKSRDISRQEIKRINKIRR | | | |
| RLVKDSNTKKAGKTGP | | | |
| KRPAATKKAGQAKKKKGGSKRPAATKKAGQ | 374 | TLEGGSPKKKRKVTLEGGSPKKKRKV | 421 |
| AKKKKGGSKRPAATKKAGQAKKKKGGSKRP | | | |
| AATKKAGQAKKKKGGSKRPAATKKAGQAKK | | | |
| KKGGSKRPAATKKAGQAKKKKSRDISRQEI | | | |
| KRINKIRRRLVKDSNTKKAGKTGP | | | |
| PKKKRKVGGSPKKKRKVGGSPKKKRKVGGS | 375 | TLEGGSPKKKRKVTLEGGSPKKKRKV | 422 |
| PKKKRKVSRDISRQEIKRINKIRRRLVKDS | | | |
| NTKKAGKTGP | | | |
| PKKKRKVGGSPKKKRKVGGSPKKKRKVGGS | 376 | TLEGGSPKKKRKVTLEGGSPKKKRKV | 423 |
| PKKKRKVSRDISRQEIKRINKIRRRLVKDS | | | |
| NTKKAGKTGP | | | |
| PAAKRVKLDGGSPAAKRVKLDGGSPAAKRV | 377 | TLEVGPKRTADSQHSTPPKTKRKVEFE | 424 |
| KLDGGSPAAKRVKLDSRDISRQEIKRINKI | | PKKKRKVTLEGGSPKKKRKV | |
| RRRLVKDSNTKKAGKTGP | | | |
| PAAKRVKLDGGSPAAKRVKLDGGSPAAKRV | 378 | TLEVGGGSGGGSKRTADSQHSTPPKTK | 425 |
| KLDGGSPAAKRVKLDGGSPAAKRVKLDGGS | | RKVEFEPKKKRKVTLEGGSPKKKRKV | |
| PAAKRVKLDSRDISRQEIKRINKIRRRLVK | | | |
| DSNTKKAGKTGP | | | |
| KRPAATKKAGQAKKKKSRDISRQEIKRINK | 379 | TLEVAEAAAKEAAAKEAAAKAKRTADS | 426 |
| IRRRLVKDSNTKKAGKTGP | | QHSTPPKTKRKVEFEPKKKRKVTLEGG | |
| | | SPKKKRKV | |
| KRPAATKKAGQAKKKKGGSKRPAATKKAGQ | 380 | TLEVGPPKKKRKVGGSKRTADSQHSTP | 427 |
| AKKKKSRDISRQEIKRINKIRRRLVKDSNT | | PKTKRKVEFEPKKKRKVTLEGGSPKKK | |
| KKAGKTGP | | RKV | |
| PAAKRVKLDGGKRTADGSEFESPKKKRKVG | 381 | TLEVGPAEAAAKEAAAKEAAAKAPAAK | 428 |
| GSSRDISRQEIKRINKIRRRLVKDSNTKKA | | RVKLDTLEGGSPKKKRKV | |
| GKTGP | | | |
| PAAKRVKLDGGKRTADGSEFESPKKKRKVP | 382 | TLEVGPGGGSGGGSGGGSPAAKRVKLD | 429 |
| PPPGSRDISRQEIKRINKIRRRLVKDSNTK | | TLEVGPKRTADSQHSTPPKTKRKVEFE | |
| KAGKTGP | | PKKKRKV | |
| PAAKRVKLDGGKRTADGSEFESPKKKRKVG | 383 | TLEVGPPKKKRKVPPPPAAKRVKLDTL | 430 |
| IHGVPAAPGSRDISRQEIKRINKIRRRLVK | | EVGGGSGGGSKRTADSQHSTPPKTKRK | |
| DSNTKKAGKTGP | | VEFEPKKKRKV | |
| PAAKRVKLDGGKRTADGSEFESPKKKRKVG | 384 | TLEVGPPAAKRVKLDTLEVAEAAAKEA | 431 |
| GGSGGGSPGSRDISRQEIKRINKIRRRLVK | | AAKEAAAKAKRTADSQHSTPPKTKRKV | |
| DSNTKKAGKTGP | | EFEPKKKRKV | |
| PAAKRVKLDGGKRTADGSEFESPKKKRKVP | 385 | TLEVGPKRTADSQHSTPPKTKRKVEFE | 432 |
| GGGSGGGSPGSRDISRQEIKRINKIRRRLV | | PKKKRKVTLEVGPPKKKRKVGGSKRTA | |
| KDSNTKKAGKTGP | | DSQHSTPPKTKRKVEFEPKKKRKV | |
| PAAKRVKLDGGKRTADGSEFESPKKKRKVA | 386 | TLEVGGGSGGGSKRTADSQHSTPPKTK | 433 |
| EAAAKEAAAKEAAAKAPGSRDISRQEIKRI | | RKVEFEPKKKRKVTLEVGPAEAAAKEA | |
| NKIRRRLVKDSNTKKAGKTGP | | AAKEAAAKAPAAKRVKLD | |
| PAAKRVKLDGGKRTADGSEFESPKKKRKVP | 387 | GSKRPAATKKAGQAKKKKTLEVGPGGG | 434 |
| GSRDISRQEIKRINKIRRRLVKDSNTKKAG | | SGGGSGGGSPAAKRVKLD | |
| KTGP | | | |
| PAAKRVKLDGGSPKKKRKVGGSSRDISRQE | 388 | GSKRPAATKKAGQAKKKKTLEVGPPKK | 435 |
| IKRINKIRRRLVKDSNTKKAGKTGP | | KRKVPPPPAAKRVKLD | |
| PAAKRVKLDPPPPKKKRKVPGSRDISRQEI | 389 | GSKRPAATKKAGQAKKKKTLEVGPPAA | 436 |
| KRINKIRRRLVKDSNTKKAGKTGP | | KRVKLD | |
| PAAKRVKLDPGRSRDISRQEIKRINKIRRR | 390 | GSPKKKRKVTLEVGPKRTADSQHSTPP | 437 |
| LVKDSNTKKAGKTGP | | KTKRKVEFEPKKKRKV | |
| PKKKRKVSRDISRQEIKRINKIRRRLVKDS | 391 | GSKRPAATKKAGQAKKKKTLEVGGGSG | 438 |
| NTKKAGKTGP | | GGSKRTADSQHSTPPKTKRKVEFEPKK | |
| | | KRKV | |
| PAAKRVKLDGGKRTADGSEFESPKKKRKVG | 392 | GSKRPAATKKAGQAKKKKGSKRPAATK | 439 |
| GSSRDISRQEIKRINKIRRRLVKDSNTKKA | | KAGQAKKKK | |
| GKTGP | | | |
| PAAKRVKLDGGKRTADGSEFESPKKKRKVG | 393 | GSKRPAATKKAGQAKKKKGSKRPAATK | 440 |
| GGSGGGSPGSRDISRQEIKRINKIRRRLVK | | KAGQAKKKK | |
| DSNTKKAGKTGP | | | |
| PKKKRKVSRQEIKRINKIRRRLVKDSNTKK | 394 | GSKRPAATKKAGQAKKKKGSKRPAATK | 441 |
| AGKTGP | | KAGQAKKKK | |
| PKKKRKVGGSPKKKRKVGGSPKKKRKVGGS | 395 | GSPKKKRKVGSPKKKRKV | 442 |
| PKKKRKVSRQEIKRINKIRRRLVKDSNTKK | | | |
| AGKTGP | | | |
| PKKKRKVGGSPKKKRKVGGSPKKKRKVGGS | 396 | GGGSGGGSKRTADSQHSTPPKTKRKVE | 443 |
| PKKKRKVGGSPKKKRKVGGSPKKKRKVSRQ | | FEPKKKRKVGSKRPAATKKAGQAKKKK | |
| EIKRINKIRRRLVKDSNTKKAGKTGP | | | |
| PAAKRVKLDSRQEIKRINKIRRRLVKDSNT | 397 | GPPKKKRKVGGSKRTADSQHSTPPKTK | 444 |
| KKAGKTGP | | RKVEFEPKKKRKVGSKRPAATKKAGQA | |
| | | KKKK | |
| PAAKRVKLDGGSPAAKRVKLDSRQEIKRIN | 398 | TGGGPGGGAAAGSGSPKKKRKVGSGSG | 445 |
| KIRRRLVKDSNTKKAGKTGP | | SKRPAATKKAGQAKKKK | |
| PAAKRVKLDGGSPAAKRVKLDGGSPAAKRV | 399 | GPKRTADSQHSTPPKTKRKVEFEPKKK | 446 |
| KLDGGSPAAKRVKLDSRQEIKRINKIRRRL | | RKVGSKRPAATKKAGQAKKKK | |
| VKDSNTKKAGKTGP | | | |
| PAAKRVKLDGGSPAAKRVKLDGGSPAAKRV | 400 | AEAAAKEAAAKEAAAKAKRTADSQHST | 447 |
| KLDGGSPAAKRVKLDGGSPAAKRVKLDGGS | | PPKTKRKVEFEPKKKRKVGSPKKKRKV | |
| PAAKRVKLDSRQEIKRINKIRRRLVKDSNT | | | |
| KKAGKTGP | | | |
| KRPAATKKAGQAKKKKSRQEIKRINKIRRR | 401 | GPPKKKRKVPPPPAAKRVKLDGGGSGG | 448 |
| LVKDSNTKKAGKTGP | | GSKRTADSQHSTPPKTKRKVEFEPKKK | |
| | | RKV | |
| TSPKKKRKVALEYPYDVPDYA | 402 | GSPAAKRVKLDGGSPAAKRVKLDGGSP | 449 |
| | | AAKRVKLDGGSPAAKRVKLDGGSPAAK | |
| | | RVKLDGGSPAAKRVKLDGPPKKKRKVG | |
| | | GSKRTADSQHSTPPKTKRKVEFEPKKK | |
| | | RKV | |
| TLESKRPAATKKAGQAKKKKAPGEYPYDVP | 403 | GSPAAKRVKLGGSPAAKRVKLGGSPKK | 450 |
| DYA | | KRKVGGSPKKKRKVTGGGPGGGAAAGS | |
| | | GSPKKKRKVGSGS | |
| GSKRPAATKKAGQAKKKKYPYDVPDYA | 404 | GSKRPAATKKAGQAKKKKGGSKRPAAT | 451 |
| | | KKAGQAKKKKGPKRTADSQHSTPPKTK | |
| | | RKVEFEPKKKRKV | |
| TLESKRPAATKKAGQAKKKKGGSKRPAATK | 405 | GSKRPAATKKAGQAKKKKGGSKRPAAT | 452 |
| KAGQAKKKKAPGEYPYDVPDYATSPKKKRK | | KKAGQAKKKKAEAAAKEAAAKEAAAKA | |
| VALEYPYDVPDYA | | KRTADSQHSTPPKTKRKVEFEPKKKRK | |
| | | V | |
| TLESKRPAATKKAGQAKKKKGGSKRPAATK | 406 | GPPKKKRKVPPPPAAKRVKLD | 453 |
| KAGQAKKKKGGSKRPAATKKAGQAKKKKGG | | | |
| SKRPAATKKAGQAKKKKTSPKKKRKVALEY | | | |
| PYDVPDYA | | | |
| TLESKRPAATKKAGQAKKKKGGSKRPAATK | 407 | GSPAAKRVKLDGGSPAAKRVKLDGGSP | 454 |
| KAGQAKKKKGGSKRPAATKKAGQAKKKKGG | | AAKRVKLDGGSPAAKRVKLDGGSPAAK | |
| SKRPAATKKAGQAKKKKGGSKRPAATKKAG | | RVKLDGGSPAAKRVKLD | |
| QAKKKKGGSKRPAATKKAGQAKKKKTSPKK | | | |
| KRKVALEYPYDVPDYA | | | |
| TLESPKKKRKVGGSPKKKRKVGGSPKKKRK | 408 | GSPAAKRVKLGGSPAAKRVKLGGSPKK | 455 |
| VGGSPKKKRKVTLESKRPAATKKAGQAKKK | | KRKVGGSPKKKRKV | |
| KAPGEYPYDVPDYA | | | |
| TLESPKKKRKVGGSPKKKRKVGGSPKKKRK | 409 | GSKRPAATKKAGQAKKKKGGSKRPAAT | 456 |
| VGGSPKKKRKVGSKRPAATKKAGQAKKKKY | | KKAGQAKKKK | |
| PYDVPDYA | | | |
| TLESPAAKRVKLDGGSPAAKRVKLDGGSPA | 410 | GSKRPAATKKAGQAKKKKGGSKRPAAT | 457 |
| AKRVKLDGGSPAAKRVKLDTLESKRPAATK | | KKAGQAKKKK | |
| KAGQAKKKKGGSKRPAATKKAGQAKKKKAP | | | |
| GEYPYDVPDYA | | | |

In some cases, an engineered CasX fusion protein includes a “Protein Transduction Domain” or PTD (also known as a CPP—cell penetrating peptide), which refers to a protein, polynucleotide, carbohydrate, or organic or inorganic compound that facilitates traversing a lipid bilayer, micelle, cell membrane, organelle membrane, or vesicle membrane. A PTD attached to another molecule, which can range from a small polar molecule to a large macromolecule and/or a nanoparticle, facilitates the molecule traversing a membrane, for example going from an extracellular space to an intracellular space, or from the cytosol to within an organelle. In some embodiments, a PTD is covalently linked to the amino terminus of an engineered CasX fusion protein. In some embodiments, a PTD is covalently linked to the carboxyl terminus of an engineered CasX fusion protein. In some cases, the PTD is inserted internally in the sequence of an engineered CasX fusion protein at a suitable insertion site. In some cases, an engineered CasX fusion protein includes (is conjugated to, is fused to) one or more PTDs (e.g., two or more, three or more, four or more PTDs). In some cases, a PTD includes one or more nuclear localization signals (NLS). Examples of PTDs include but are not limited to peptide transduction domain of HIV TAT comprising YGRKKRRQRRR (SEQ ID NO: 458), RKKRRQRR (SEQ ID NO: 459); YARAAARQARA (SEQ ID NO: 460); THRLPRRRRRR (SEQ ID NO: 461); and GGRRARRRRRR (SEQ ID NO: 462); a polyarginine sequence comprising a number of arginines sufficient to direct entry into a cell (e.g., 3, 4, 5, 6, 7, 8, 9, 10, or 10-50 arginines, SEQ ID NO: 463); a VP22 domain (Zender et al. (2002) Cancer Gene Ther. 9(6):489-96); an Drosophila Antennapedia protein transduction domain (Noguchi et al. (2003) Diabetes 52(7): 1732-1737); a truncated human calcitonin peptide (Trehin et al. (2004) Pharm. Research 21:1248-1256); polylysine (Wender et al. (2000) Proc. Natl. Acad. Sci. USA 97: 13003-13008); RRQRRTSKLMKR (SEQ ID NO: 464); Transportan GWTLNSAGYLLGKINLKALAALAKKIL (SEQ ID NO: 465); KALAWEAKLAKALAKALAKHLAKALAKALKCEA (SEQ ID NO: 466); and RQIKIWFQNRRMKWKK (SEQ ID NO: 467). In some embodiments, the PTD is an activatable CPP (ACPP) (Aguilera et al. (2009) Integr Biol (Camb) June; 1(5-6): 371-381). ACPPs comprise a polycationic CPP (e.g., Arg9 or “R9”) connected via a cleavable linker to a matching polyanion (e.g., Glu9 or “E9”), which reduces the net charge to nearly zero and thereby inhibits adhesion and uptake into cells. Upon cleavage of the linker, the polyanion is released, locally unmasking the polyarginine and its inherent adhesiveness, thus “activating” the ACPP to traverse the membrane.

In some embodiments, an engineered CasX fusion protein can include a CasX protein that is linked to a heterologous polypeptide (a heterologous amino acid sequence) via a linker polypeptide (e.g., one or more linker polypeptides). In some embodiments, an engineered CasX fusion protein can be linked at the C-terminal and/or N-terminal end to a heterologous polypeptide (fusion partner) via a linker polypeptide (e.g., one or more linker polypeptides). The linker polypeptide may have any of a variety of amino acid sequences. Proteins can be joined by a spacer peptide, generally of a flexible nature, although other chemical linkages are not excluded. Suitable linkers include polypeptides of between 4 amino acids and 40 amino acids in length, or between 4 amino acids and 25 amino acids in length. The linking peptides may have virtually any amino acid sequence, bearing in mind that the preferred linkers will have a sequence that results in a generally flexible peptide. The use of small amino acids, such as glycine and alanine, are of use in creating a flexible peptide. The creation of such sequences is routine to those of skill in the art. A variety of different linkers are commercially available and are considered suitable for use. In some embodiments, the one or more fusion proteins are linked to the engineered CasX protein or to adjacent fusion proteins with a linker peptide wherein the linker peptide is selected from the group consisting of RS, (G)n (SEQ ID NO: 468), (GS)n (SEQ ID NO: 469), (GSGGS)n (SEQ ID NO: 470), (GGSGGS)n (SEQ ID NO: 471), (GGGS)n (SEQ ID NO: 472), GGSG (SEQ ID NO: 473), GGSGG (SEQ ID NO: 474), GSGSG (SEQ ID NO: 475), GSGGG (SEQ ID NO: 476), GGGSG (SEQ ID NO: 477), GSSSG (SEQ ID NO: 478), GPGP (SEQ ID NO: 479), GGP, PPP, PPAPPA (SEQ ID NO: 480), PPPG (SEQ ID NO: 481), PPPGPPP (SEQ ID NO: 482), PPP(GGGS)n (SEQ ID NO: 483), (GGGS)nPPP (SEQ ID NO: 484), AEAAAKEAAAKEAAAKA (SEQ ID NO: 485), and TPPKTKRKVEFE (SEQ ID NO: 486), where n is 1 to 5. The ordinarily skilled artisan will recognize that design of a peptide conjugated to any elements described above can include linkers that are all or partially flexible, such that the linker can include a flexible linker as well as one or more portions that confer less flexible structure.

### V. Methods of Making Engineered CasX Proteins and ERS

The engineered CasX proteins and ERS of the disclosure may be designed and constructed through a variety of methods, as described herein. In some embodiments, the method comprises designing, building and testing a comprehensive set of mutations to a starting biomolecule to produce a library of biomolecule variants; for example, a library of engineered CasX proteins or engineered ERS scaffolds. The methods of the disclosure can encompass making all possible substitutions, as well as all possible small insertions, and all possible deletions of amino acids (in the case of proteins) or nucleotides (in the case of RNA or DNA), or the swapping of domains or subdomains to the starting biomolecule in order to create libraries that are then evaluated for functional changes, and this information used to construct one or more additional libraries. Such iterative construction and evaluation of variants may lead, for example, to identification of mutational themes that lead to certain functional outcomes, such as regions of the protein or gRNA that, when mutated in a certain way, lead to one or more improved functions. Layering of such identified mutations may then further improve function, for example through additive or synergistic interactions. The methods of the disclosure comprise library design, library construction, and library screening. In some embodiments, multiple rounds of design, construction, and screening are undertaken.

a. Library Design

In some embodiments, the methods to create libraries of mutagenized CasX and ERS are the methods of Examples 1-7 and 11. In some embodiments, the biomolecule of the library comprises a protein or a ribonucleic acid (RNA) molecule, wherein the mutagenized monomer units are amino acids or ribonucleotides, respectively. The fundamental units of biomolecule mutation comprise either: (1) exchanging one monomer for another monomer of different identity (substitutions); (2) inserting one or more additional monomers in the biomolecule (insertions); or (3) removing one or more monomers from the biomolecule (deletions). Libraries comprising substitutions, insertions, and deletions, alone or in combination, to any one or more monomers within any biomolecule described herein, are considered within the scope of the invention.

In an exemplary embodiment, and as described in Example 1, the disclosure provides CasX proteins derived from CasX 515 in which engineered CasX were designed using a Markov Chain Monte Carlo (MCMC)-directed evolution simulation (Biswas S et al. Low-N protein engineering with data-efficient deep learning. Nature Methods. 18(4):389-396 (2021)). In the method, a codon within CasX 515 was selected and randomly replaced with a codon encoding a different amino acid, such that there was an equal probability of the selected amino acid to be replaced with any of the alternative 19 amino acids. This process was then repeated up to sixteen times, resulting in a simulated mutagenized protein sequence. Then, the predicted fitness of the mutagenized protein sequence was determined using a machine learning model to virtually screen the simulated protein either to discard the simulated protein or to construct and validate the simulated protein experimentally. In the method, the process of mutagenesis and simulated screening was repeated until a desired number of sequences, each containing a desired number of single mutations, were obtained, which were subsequently assayed to identify those engineered CasX with improved characteristics.

In some embodiments, a library design comprises enumerating all possible mutations for each of one or more target monomers in a biomolecule. As used herein, a “target monomer” refers to a monomer in a biomolecule polymer that is targeted for mutagenesis with the substitutions, insertions and deletions described herein. For example, a target monomer can be an amino acid at a specified position in a protein, or a nucleotide at a specified position in an RNA. In some embodiments, a library of mutated sequences is created by mutation at each consecutive position in the protein or RNA. In other embodiments, a biomolecule can have at least 1, 2, 3, 4, 5, 6, 7, 8, 9, 10, 20, 30, 40, 50, 100 or more target monomers that are systematically mutated to produce a library of biomolecule variants. In some embodiments, every monomer in a biomolecule is a target monomer. For example, in a parental CasX protein where there are two target amino acids, a library design comprises enumerating the 40 possible mutations at each of the two target amino acids. In a further example, in a library of an RNA where there are four target nucleotides, the library design comprises enumerating the 8 possible mutations at each of the four target nucleotides. In some embodiments, each target monomer of a biomolecule is independently randomly selected or selected by intentional design. Thus, in some embodiments, a library comprises random variants, or variants that were designed, or variants comprising random mutations and designed mutations within a single biomolecule, or any combinations thereof.

In some embodiments, the assembled library is then assayed to assess the comprehensive set of mutations to a biomolecule, encompassing the substitutions, as well as insertions and deletions of amino acids (in the case of proteins) or nucleotides (in the case of RNA). The construction and functional readout of these mutations can be achieved with a variety of established molecular biology methods. In some embodiments, the library comprises a subset of all possible modifications to monomers. For example, in some embodiments, a library collectively represents a single modification of one monomer, for at least some percentage of the total monomer locations in a biomolecule, wherein each single modification is selected from the group consisting of substitution, single insertion, and single deletion. In some embodiments, the library collectively represents the single modification of one monomer for at least 1%, at least 5%, at least 10%, at least 20%, at least 30%, at least 40%, at least 50%, at least 60%, at least 70%, at least 80%, at least 90%, at least 95%, or up to 100% of the total monomer locations in a starting biomolecule. In certain embodiments, for a certain percentage of the total monomer locations in a starting biomolecule, the library collectively represents each possible single modification of one monomer, such as all possible substitutions with the 19 other naturally occurring amino acids (for a protein) or 3 other naturally occurring ribonucleotides (for RNA), insertion of each of the 20 naturally occurring amino acids (for a protein) or 4 naturally occurring ribonucleotides (for RNA), or deletion of the monomer. In still further embodiments, insertion at each location is independently greater than one monomer, for example insertion of two or more, three or more, or four or more monomers, or insertion of between one to four, between two to four, or between one to three monomers. In some embodiments, deletion at a location is independently greater than one monomer, for example, deletion of two or more, three or more, or four or more monomers, or deletion of between one to four, between two to four, or between one to three monomers. Examples of such libraries of engineered CasX and ERS are described in Examples 1-7 and 11.

In some embodiments, the biomolecule is a protein and the individual monomers are amino acids. In those embodiments where the biomolecule is a protein, the number of possible mutations at each monomer (amino acid) position in the protein comprise 19 amino acid substitutions, 20 amino acid insertions and 1 amino acid deletion, leading to a total of 40 possible mutations per amino acid in the protein.

In some embodiments, a library of engineered CasX proteins comprising insertions is a 1 amino acid insertion library, a 2 amino acid insertion library, a 3 amino acid insertion library, a 4 amino acid insertion library, a 5 amino acid insertion library, a 6 amino acid insertion library, a 7 amino acid insertion library, an 8 amino acid insertion library, a 9 amino acid insertion library, or a 10 amino acid insertion library. In some embodiments, a library of engineered CasX proteins comprising insertions comprises between 1 and 10 amino acid insertions. In some embodiments, a library of engineered CasX proteins comprising deletions is a 1 amino acid deletion library, a 2 amino acid deletion library, a 3 amino acid deletion library, a 4 amino acid deletion library, a 5 amino acid deletion library, a 6 amino acid deletion library, a 7 amino acid deletion library, an 8 amino acid deletion library, a 9 amino acid deletion library, or a 10 amino acid deletion library. In some embodiments, a library of engineered CasX proteins comprising deletions comprises between 1 and 10 amino acid deletions. In some embodiments, a library of engineered CasX proteins comprising substitutions is a 1 amino acid substitution library, a 2 amino acid substitution library, a 3 amino acid substitution library, a 4 amino acid substitution library, a 5 amino acid substitution library, a 6 amino acid substitution library, a 7 amino acid substitution library, an 8 amino acid substitution library, a 9 amino acid substitution library, or a 10 amino acid insertion library. In some embodiments, a library of engineered CasX proteins comprising substitutions comprises between 1 and 10 amino acid substitutions.

In some embodiments, the biomolecule is RNA. In those embodiments where the biomolecule is RNA, the number of possible DME mutations at each monomer (ribonucleotide) position in the RNA comprises 3 nucleotide substitutions, 4 nucleotide insertions, and 1 nucleotide deletion, leading to a total of 8 possible mutations per nucleotide.

In some embodiments of the methods, mutations are incorporated into double-stranded DNA encoding the biomolecule. This DNA can be maintained and replicated in a standard cloning vector, for example a bacterial plasmid, referred to herein as the target plasmid. An exemplary target plasmid contains a DNA sequence encoding the starting biomolecule that will be subjected to mutagenesis, a bacterial origin of replication, and a suitable antibiotic resistance expression cassette. In some embodiments, the antibiotic resistance cassette confers resistance to kanamycin, ampicillin, spectinomycin, bleomycin, streptomycin, erythromycin, tetracycline or chloramphenicol. In some embodiments, the antibiotic resistance cassette confers resistance to kanamycin.

A library comprising said variants can be constructed in a variety of ways. In certain embodiments, plasmid recombineering is used to construct a library. Such methods can use DNA oligonucleotides encoding one or more mutations to incorporate said mutations into a plasmid encoding the reference biomolecule. For biomolecule variants with a plurality of mutations, in some embodiments more than one oligonucleotide is used. In some embodiments, the DNA oligonucleotides encode one or more mutations wherein the mutation region is flanked by between 10 and 100 nucleotides of homology to the target plasmid, both 5′ and 3′ to the mutation. Such oligonucleotides can in some embodiments be commercially synthesized and used in PCR amplification. An exemplary template for an oligonucleotide encoding a mutation is provided below:
5′-(N)₁₀₋₁₀₀-Mutation-(N′)₁₀₋₁₀₀-3′

In this exemplary oligonucleotide design, the Ns represent a sequence identical to the target plasmid, referred to herein as the homology arms. When a particular monomer in the biomolecule is targeted for mutation, these homology arms directly flank the DNA encoding the monomer in the target plasmid. In some exemplary embodiments where the biomolecule undergoing mutagenesis is a protein, 40 different oligonucleotides, using the same set of homology arms, are used to encode the enumerated 40 different amino acid mutations for each amino acid residue in the protein that is targeted for mutagenesis. When the mutation is of a single amino acid, the region encoding the desired mutation or mutations comprises three nucleotides encoding an amino acid (for substitutions or single insertions), or zero nucleotides (for deletions). In some embodiments, the oligonucleotide encodes insertion of greater than one amino acid. For example, wherein the oligonucleotide encodes the insertion of X amino acids, the region encoding the desired mutation comprises 3*X nucleotides encoding the X amino acids. In some embodiments, the mutation region encodes more than one mutation, for example mutations to two or more monomers of a biomolecule that are in close proximity (e.g., next to each other, or within 1, 2, 3, 4, 5, 6, 7, 8, 9, or 10, or more monomers of each other).

In some exemplary embodiments where the biomolecule undergoing mutagenesis is an RNA, 8 different oligonucleotides, using the same set of homology arms, encode 8 different single nucleotide mutations for each nucleotide in the RNA that is targeted for mutagenesis. When the mutation is of a single ribonucleotide, the region of the oligo encoding the mutations can consist of the following nucleotide sequences: one nucleotide specifying a nucleotide (for substitutions or insertions), or zero nucleotides (for deletions). In some embodiments, the oligonucleotides are synthesized as single stranded DNA oligonucleotides. In some embodiments, all oligonucleotides targeting a particular amino acid or nucleotide of a biomolecule subjected to mutagenesis are pooled.

b. Library Screening

Any appropriate method for screening or selecting a library is envisaged as following within the scope of the inventions. High throughput methods may be used to evaluate large libraries with thousands of individual mutations. In some embodiments, the throughput of the library screening or selection assay has a throughput that is in the millions of individual cells. In some embodiments, assays utilizing living cells are preferred because phenotype and genotype are physically linked in living cells by nature of being contained within the same lipid bilayer. Living cells can also be used to directly amplify sub-populations of the overall library. Exemplary methods of screening libraries are described in Examples 1-7 and 11.

In some embodiments, libraries that have been screened or selected for highly functional variants are further characterized. In some embodiments, further characterizing the library comprises analyzing variants individually through sequencing, such as Sanger sequencing, to identify the specific mutation or mutations that gave rise to the highly functional variant. Individual mutant variants of the biomolecule can be isolated through standard molecular biology techniques for later analysis of function. In some embodiments, further characterizing the library comprises high throughput sequencing of both the library and the one or more libraries of highly functional variants. This approach may, in some embodiments, allow for the rapid identification of mutations that are over-represented in the one or more libraries of highly functional variants compared to the naïve library. Without wishing to be bound by any theory, mutations that are over-represented in the one or more libraries of highly functional variants are likely to be responsible for the activity of the highly functional variants. In some embodiments, further characterizing the library comprises both sequencing of individual variants and high throughput sequencing of both a naïve library and the one or more libraries of highly mutagenized variants.

High throughput sequencing can produce high throughput data indicating the functional effect of the library members. In embodiments wherein one or more libraries represents every possible mutation of every monomer location, such high throughput sequencing can evaluate the functional effect of every possible mutation. Such sequencing can also be used to evaluate one or more highly functional sub-populations of a given library, which in some embodiments may lead to identification of mutations that result in improved function.

c. Production of Engineered CasX and ERS

An engineered CasX protein of the present disclosure may be produced in vitro by eukaryotic cells or by prokaryotic cells transformed with encoding vectors (described below) using standard cloning and molecularly biology techniques or as described in the Examples. The particular sequence and the manner of preparation will be determined by convenience, economics, purity required, and the like. In some embodiments, a construct is first prepared containing the DNA sequence encoding the engineered CasX. Exemplary methods for the preparation of such constructs are described in the Examples. The construct is then used to create an expression vector suitable for transforming a host cell, such as a prokaryotic or eukaryotic host cell for the expression and recovery of the protein. Where desired, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), or yeast cells, or other eukaryotic cells known in the art suitable for the production of recombinant products.

An engineered CasX protein of the disclosure may also be isolated and purified in accordance with conventional methods of recombinant synthesis. A lysate may be prepared of the expression host and the lysate purified using high performance liquid chromatography (HPLC), exclusion chromatography, gel electrophoresis, affinity chromatography, or other purification technique. For the most part, the compositions which are used will comprise 80% or more by weight of the desired product, more usually 90% or more by weight, preferably 95% or more by weight, and for therapeutic purposes, usually 99.5% or more by weight, in relation to contaminants related to the method of preparation of the product and its purification.

In the case of production of the ERS (and linked targeting sequences) of the present disclosure, recombinant expression vectors encoding the ERS can be transcribed in vitro, for example using T7 promoter regulatory sequences and T7 polymerase in order to produce the ERS, which can then be recovered by conventional methods; e.g., purification via gel electrophoresis as described in the Examples. Alternatively, the ERS can be prepared synthetically. Once synthesized, the ERS may be utilized in the gene editing pair systems to directly contact and modify a target nucleic acid or may be introduced into a cell by any of the well-known techniques for introducing nucleic acids into cells (e.g., microinjection, electroporation, transfection, etc.).

### VI. Polynucleotides and Vectors

In another aspect, the present disclosure relates to polynucleotides encoding the engineered CasX and ERS that have utility in the editing of the target nucleic acid in a cell. In some embodiments, the disclosure provides polynucleotides encoding the engineered CasX proteins and the polynucleotides of the ERS of any of the system embodiments described herein. In some embodiments, the disclosure provides a polynucleotide sequence encoding the engineered CasX of any of the embodiments described herein, including the engineered CasX of SEQ ID NOS: 247-294, 24916-49628, 49746-49747, or 49871-49873 or sequences having at least about 80%, at least about 90%, at least about 95%, at least about 96%, at least about 97%, at least about 98%, or at least about 99% sequence identity to a sequence thereto. In some embodiments, the disclosure provides an isolated polynucleotide sequence encoding an ERS sequence of any of the embodiments described herein, including the sequences of SEQ ID NOS: 156, 739-907, 11568-22227, 23572-24915, and 49719-49735, together with targeting sequences capable of hybridizing with the target nucleic acid to be modified.

In other aspects, the disclosure relates to methods to produce polynucleotide sequences encoding the engineered CasX, or the ERS of any of the embodiments described herein, including homologous variants thereof, as well as methods to express the proteins expressed or ERS transcribed by the polynucleotide sequences. In general, the methods include producing a polynucleotide sequence coding for the engineered CasX, or the ERS of any of the embodiments described herein and incorporating the encoding gene into an expression vector appropriate for a host cell. Standard recombinant techniques in molecular biology can be used to make the polynucleotides and expression vectors of the present disclosure. For production of the encoded reference CasX, the engineered CasX, or the ERS of any of the embodiments described herein, the methods include transforming an appropriate host cell with an expression vector comprising the encoding polynucleotide, and culturing the host cell under conditions causing or permitting the resulting reference CasX, the engineered CasX, or the ERS of any of the embodiments described herein to be expressed or transcribed in the transformed host cell, thereby producing the engineered CasX, or the ERS, which are recovered by methods described herein or by standard purification methods known in the art or as described in the Examples.

In accordance with the disclosure, nucleic acid sequences that encode the engineered CasX, or the ERS of any of the embodiments described herein (or their complement) are used to generate recombinant DNA molecules that direct the expression in appropriate host cells. Several cloning strategies are suitable for performing the present disclosure, many of which are used to generate a construct that comprises a gene coding for a composition of the present disclosure, or its complement. In some embodiments, the cloning strategy is used to create a gene that encodes a construct that comprises nucleotides encoding the engineered CasX or the ERS that is used to transform a host cell for expression of the composition.

In some approaches, a construct is first prepared containing the DNA sequence encoding an engineered CasX or an ERS. Exemplary methods for the preparation of such constructs are described in the Examples. The construct is then used to create an expression vector suitable for transforming a host cell, such as a prokaryotic or eukaryotic host cell for the expression and recovery of the protein construct, in the case of the engineered CasX, or the ERS. Where desired, the host cell is an E. coli. In other embodiments, the host cell is a eukaryotic cell. The eukaryotic host cell can be selected from Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO), or yeast cells, or other eukaryotic cells known in the art suitable for the production of recombinant products. Exemplary methods for the creation of expression vectors, the transformation of host cells and the expression and recovery of the engineered CasX or the ERS are described in the Examples.

The gene encoding the engineered CasX, or the ERS construct can be made in one or more steps, either fully synthetically or by synthesis combined with enzymatic processes, such as restriction enzyme-mediated cloning, PCR and overlap extension, including methods more fully described in the Examples. The methods disclosed herein can be used, for example, to ligate sequences of polynucleotides encoding the various components (e.g., engineered CasX and ERS) genes of a desired sequence. Genes encoding polypeptide compositions are assembled from oligonucleotides using standard techniques of gene synthesis.

In some embodiments, the nucleotide sequence encoding an engineered CasX protein is codon optimized. This type of optimization can entail a mutation of an encoding nucleotide sequence to mimic the codon preferences of the intended host organism or cell while encoding the same engineered CasX protein. Thus, the codons can be changed, but the encoded protein remains unchanged. For example, if the intended target cell of the engineered CasX protein is a human cell, a human codon-optimized encoding nucleotide sequence could be used. As another non-limiting example, if the intended host cell was a mouse cell, then a mouse codon-optimized encoding nucleotide sequence could be generated. As another non-limiting example, if the intended host cell was a prokaryotic cell (e.g., E. coli), then a prokaryotic codon-optimized encoding nucleotide sequence could be generated. The gene design can be performed using algorithms that optimize codon usage and amino acid composition appropriate for the host cell utilized in the production of the engineered CasX. In one method of the disclosure, a library of polynucleotides encoding the engineered CasX or ERS components is created and then assembled, as described above and assayed to confirm that the variants retain functional properties. The resulting genes are then used to transform a host cell and produce and recover the engineered CasX or the ERS compositions for evaluation of its properties, as described herein.

In some embodiments, the nucleotide sequence encoding the engineered CasX protein is depleted or devoid of CpG motifs. In some embodiments, the CpG content of the engineered CasX is less than about 10%, less than about 5%, or less than about 1% CpG. In some embodiments, the sequence encoding the engineered CasX protein depleted or devoid of CpG motifs comprises a sequence selected from the group consisting of SEQ ID NOS: 49850-49861.

In some embodiments, the nucleotide sequence encoding the ERS is depleted or devoid of CpG motifs. In some embodiments, the CpG content of the ERS is less than about 10%, less than about 5%, or less than about 1% CpG. In some embodiments, the nucleotide encoding the ERS depleted or devoid of CpG motifs comprises a sequence selected from the group consisting of SEQ ID NOS: 535-556.

In some embodiments, a nucleotide sequence encoding a ERS is operably linked to a control element; e.g., a transcriptional control element, such as a promoter. In some embodiments, a nucleotide sequence encoding an engineered CasX protein is operably linked to a control element; e.g., a transcriptional control element, such as a promoter. In some cases, the promoter is a constitutively active promoter. In some cases, the promoter is a regulatable promoter. In some cases, the promoter is an inducible promoter. In some cases, the promoter is a tissue-specific promoter. In some cases, the promoter is a cell type-specific promoter. In some cases, the transcriptional control element (e.g., the promoter) is functional in a targeted cell type or targeted cell population. For example, in some cases, the transcriptional control element can be functional in eukaryotic cells; e.g., neurons, spinal motor neurons, medium spiny neurons, cortical neurons, striatal neurons, oligodendrocytes, or glial cells.

Non-limiting examples of Pol II promoters operably linked to the polynucleotide encoding the engineered CasX of the disclosure include, but are not limited to EF-1alpha, EF-1alpha core promoter, Jens Tornoe (JeT), promoters from cytomegalovirus (CMV), CMV immediate early (CMVIE), CMV enhancer, herpes simplex virus (HSV) thymidine kinase, early and late simian virus 40 (SV40), the SV40 enhancer, long terminal repeats (LTRs) from retrovirus, mouse metallothionein-I, adenovirus major late promoter (Ad MLP), CMV promoter full-length promoter, the minimal CMV promoter, the chicken β-actin promoter (CBA), CBA hybrid (CBh), chicken β-actin promoter with cytomegalovirus enhancer (CB7), chicken beta-Actin promoter and rabbit beta-Globin splice acceptor site fusion (CAG), the rous sarcoma virus (RSV) promoter, the HIV-Ltr promoter, the hPGK promoter, the HSV TK promoter, a 7SK promoter, the Mini-TK promoter, the human synapsin I (SYN) promoter which confers neuron-specific expression, beta-actin promoter, super core promoter 1 (SCP1), the Mecp2 promoter for selective expression in neurons, the minimal IL-2 promoter, the Rous sarcoma virus enhancer/promoter (single), the spleen focus-forming virus long terminal repeat (LTR) promoter, the TBG promoter, promoter from the human thyroxine-binding globulin gene (Liver specific), the PGK promoter, the human ubiquitin C promoter (UBC), the UCOE promoter (Promoter of HNRPA2B1-CBX3), the synthetic CAG promoter, the Histone H2 promoter, the Histone H3 promoter, the U1a1 small nuclear RNA promoter (226 nt), the U1a1 small nuclear RNA promoter (226 nt), the U1b2 small nuclear RNA promoter (246 nt) 26, the GUSB promoter, the CBh promoter, rhodopsin (Rho) promoter, silencing-prone spleen focus forming virus (SFFV) promoter, a human H1 promoter (H1), a POL1 promoter, the TTR minimal enhancer/promoter, the b-kinesin promoter, mouse mammary tumor virus long terminal repeat (LTR) promoter, the human eukaryotic initiation factor 4A (EIF4A1) promoter, the ROSA26 promoter, the glyceraldehyde 3-phosphate dehydrogenase (GAPDH) promoter, tRNA promoters, and truncated versions and sequence variants of the foregoing. In a particular embodiment, the Pol II promoter is EF-1alpha, wherein the promoter enhances transfection efficiency, the transgene transcription or expression of the CRISPR nuclease, the proportion of expression-positive clones and the copy number of the episomal vector in long-term culture.

Non-limiting examples of Pol III promoters operably linked to the polynucleotide encoding the ERS of the disclosure include, but are not limited to U6, mini U6, U6 truncated promoters, 7SK, and H1 variants, BiH1 (Bidrectional H1 promoter), BiU6, Bi7SK, BiH1 (Bidirectional U6, 7SK, and H1 promoters), gorilla U6, rhesus U6, human 7SK, human H1 promoters, and truncated versions and sequence variants thereof. In the foregoing embodiment, the Pol III promoter enhances the transcription of the ERS. In a particular embodiment, the Pol III promoter is U6, wherein the promoter enhances expression of the CRISPR ERS. In another particular embodiment, the promoter linked to the gene encoding the tropism factor is CMV promoter. Experimental details and data for the use of such promoters are provided in the examples.

Recombinant expression vectors of the disclosure can also comprise accessory elements that facilitate robust expression of engineered CasX proteins and the ERS of the disclosure. For example, recombinant expression vectors can include one or more of a polyadenylation signal (poly(A), an intronic sequence or a post-transcriptional regulatory element such as a woodchuck hepatitis post-transcriptional regulatory element (WPTRE). Exemplary poly(A) sequences include hGH poly(A) signal (short), HSV TK poly(A) signal, synthetic polyadenylation signals, SV40 poly(A) signal, β-globin poly(A) signal and the like. In some embodiments, a recombinant expression vector encoding an engineered CasX comprises a poly(A) tail of 80 or more adenine nucleotides. A person of ordinary skill in the art will be able to select suitable elements to include in the recombinant expression vectors described herein.

Selection of the appropriate vector and promoter is well within the level of ordinary skill in the art, as it related to controlling expression, e.g., for modifying a protein involved in antigen processing, antigen presentation, antigen recognition, and/or antigen response and/or its regulatory element. The expression vector may also contain a ribosome binding site for translation initiation and a transcription terminator. The expression vector may also include appropriate sequences for amplifying expression. The expression vector may also include nucleotide sequences encoding protein tags (e.g., 6×His tag, hemagglutinin tag, FLAG tag, fluorescent protein, etc.) that can be fused to the engineered CasX protein, thus resulting in a chimeric CasX protein that are used for purification or detection.

In some embodiments, provided herein are one or more recombinant expression vectors comprising one or more of: (i) a nucleotide sequence that encodes a ERS that hybridizes to a target sequence of the locus of the targeted genome (e.g., configured as a single or dual guide) operably linked to a promoter that is operable in a target cell such as a eukaryotic cell; and (ii) a nucleotide sequence encoding an engineered CasX protein operably linked to a promoter that is operable in a target cell such as a eukaryotic cell.

The polynucleotide sequence(s) are inserted into the vector by a variety of procedures. In general, DNA is inserted into an appropriate restriction endonuclease site(s) using techniques known in the art. Vector components generally include, but are not limited to, one or more of a signal sequence, an origin of replication, one or more marker genes, an enhancer element, a promoter, and a transcription termination sequence. Construction of suitable vectors containing one or more of these components employs standard ligation techniques which are known to the skilled artisan. Such techniques are well known in the art and well described in the scientific and patent literature. Various vectors are publicly available. The vector may, for example, be in the form of a plasmid, cosmid, viral particle, or phage that may conveniently be subjected to recombinant DNA procedures, and the choice of vector will often depend on the host cell into which it is to be introduced. Thus, the vector may be an autonomously replicating vector, i.e., a vector, which exists as an extrachromosomal entity, the replication of which is independent of chromosomal replication, e.g., a plasmid. Alternatively, the vector may be one which, when introduced into a host cell, is integrated into the host cell genome and replicated together with the chromosome(s) into which it has been integrated. Once introduced into a suitable host cell, expression of the protein involved in antigen processing, antigen presentation, antigen recognition, and/or antigen response can be determined using any nucleic acid or protein assay known in the art. For example, the presence of transcribed mRNA of the engineered CasX can be detected and/or quantified by conventional hybridization assays (e.g., Northern blot analysis), amplification procedures (e.g. RT-PCR), SAGE (U.S. Pat. No. 5,695,937), and array-based technologies (see e.g., U.S. Pat. Nos. 5,405,783, 5,412,087 and 5,445,934), using probes complementary to any region of the polynucleotide.

The polynucleotides and recombinant expression vectors can be delivered to the target host cells by a variety of methods. Such methods include, but are not limited to, viral infection, transfection, lipofection, electroporation, calcium phosphate precipitation, polyethyleneimine (PEI)-mediated transfection, DEAE-dextran mediated transfection, microinjection, liposome-mediated transfection, particle gun technology, nucleofection, direct addition by cell penetrating CasX proteins that are fused to or recruit donor DNA, cell squeezing, calcium phosphate precipitation, direct microinjection, nanoparticle-mediated nucleic acid delivery, and using the commercially available TransMessenger® reagents from Qiagen, Stemfect™ RNA Transfection Kit from Stemgent, and TransIT®-mRNA Transfection Kit from Mirus Bio LLC, Lonza nucleofection, Maxagen electroporation and the like.

In some embodiments, the present disclosure provides vectors comprising the polynucleotides encoding the engineered CasX or ERS selected from the group consisting of a retroviral vector, a lentiviral vector, an adenoviral vector, an adeno-associated viral (AAV) vector, a virus-like particle (VLP), a herpes simplex virus (HSV) vector, a plasmid, a minicircle, a nanoplasmid, a DNA vector, an RNA vector, or a CasX delivery particle (XDP). In some embodiments, the disclosure provides a recombinant expression vector comprising a nucleotide sequence encoding an engineered CasX protein and a nucleotide sequence encoding a ERS. In other embodiments, the nucleotide sequence encoding the engineered CasX protein and the nucleotide sequence encoding the ERS are provided in separate vectors.

In some embodiments, a recombinant expression vector of the present disclosure is a recombinant adeno-associated virus (AAV) vector. AAV is a small (20 nm), nonpathogenic virus that is useful in treating human diseases in situations that employ a viral vector for delivery to a cell such as a eukaryotic cell, either in vivo or ex vivo for cells to be prepared for administration to a subject. A construct is generated, for example, encoding any of the engineered CasX proteins and ERS embodiments as described herein, and optionally a donor template, and can be flanked with AAV inverted terminal repeat (ITR) sequences, thereby enabling packaging of the AAV vector into an AAV viral particle.

An “AAV” vector may refer to the naturally occurring wild-type virus itself or derivatives thereof. The term covers all subtypes, serotypes and pseudotypes, and both naturally occurring and recombinant forms, except where required otherwise. As used herein, the term “serotype” refers to an AAV which is identified by and distinguished from other AAVs based on capsid protein reactivity with defined antisera, e.g., there are many known serotypes of primate AAVs. In some embodiments, the AAV vector is selected from AAV1, AAV2, AAV3, AAV4, AAV5, AAV6, AAV7, AAV8, AAV9, AAV 10, AAV12, AAV 9.45, AAV 9.61, AAV 44.9, AAV-Rh74 (Rhesus macaque-derived AAV), and AAVRh10, and modified capsids of these serotypes. For example, serotype AAV-2 is used to refer to an AAV which contains capsid proteins encoded from the cap gene of AAV-2 and a genome containing 5′ and 3′ ITR sequences from the same AAV-2 serotype. Pseudotyped AAV refers to an AAV that contains capsid proteins from one serotype and a viral genome including 5′-3′ ITRs of a second serotype. Pseudotyped rAAV would be expected to have cell surface binding properties of the capsid serotype and genetic properties consistent with the ITR serotype. Pseudotyped recombinant AAV (rAAV) are produced using standard techniques described in the art. As used herein, for example, rAAV1 may be used to refer an AAV having both capsid proteins and 5′-3′ ITRs from the same serotype or it may refer to an AAV having capsid proteins from serotype 1 and 5′-3′ ITRs from a different AAV serotype, e.g., AAV serotype 2. For each example illustrated herein the description of the vector design and production describes the serotype of the capsid and 5′-3′ ITR sequences.

An “AAV virus” or “AAV viral particle” refers to a viral particle composed of at least one AAV capsid protein (preferably by all of the capsid proteins of a wild-type AAV) and an encapsidated polynucleotide. If the particle additionally comprises a heterologous polynucleotide (i.e., a polynucleotide other than a wild-type AAV genome to be delivered to a mammalian cell), it is typically referred to as “rAAV”. An exemplary heterologous polynucleotide is a polynucleotide comprising an engineered CasX protein and/or ERS and, optionally, a donor template of any of the embodiments described herein.

By “adeno-associated virus inverted terminal repeats” or “AAV ITRs” is meant the art recognized regions found at each end of the AAV genome which function together in cis as origins of DNA replication and as packaging signals for the virus. AAV ITRs, together with the AAV rep coding region, provide for the efficient excision and rescue from, and integration of a nucleotide sequence interposed between two flanking ITRs into a mammalian cell genome.

The nucleotide sequences of AAV ITR regions are known. See, for example Kotin, R. M. (1994) Human Gene Therapy 5:793-801; Berns, K. I. “Parvoviridae and their Replication” in Fundamental Virology, 2nd Edition, (B. N. Fields and D. M. Knipe, eds.). As used herein, an AAV ITR need not have the wild-type nucleotide sequence depicted, but may be altered, e.g., by the insertion, deletion or substitution of nucleotides. Additionally, the AAV ITR may be derived from any of several AAV serotypes, including without limitation, AAV1, AAV2, AAV3, AAV4, AAV5, AAV6, AAV7, AAV8, AAV9, AAV10, AAV12, AAV 9.45, AAV 9.61, AAV-Rh74, and AAVRh10, and modified capsids of these serotypes. Furthermore, 5′ and 3′ ITRs which flank a selected transgene nucleotide sequence in an AAV vector need not necessarily be identical or derived from the same AAV serotype or isolate, so long as they function as intended; i.e., to allow for excision and rescue of the sequence of interest from a host cell genome or vector, and to allow integration of the heterologous sequence into the recipient cell genome when AAV Rep gene products are present in the cell. Use of AAV serotypes for integration of heterologous sequences into a host cell is known in the art (see, e.g., WO2018195555A1 and US20180258424A1, incorporated by reference herein). In one particular embodiment, the ITRs are derived from serotype AAV1. In a particular embodiment, the ITR regions flanking the transgene of the embodiments are derived from AAV2; the 5′ ITR of the transgene of the AAV constructs of the disclosure has the sequence CCTGCAGGCAGCTGCGCGCTCGCTCGCTCACTGAGGCCGCCCGGGCGTCGGGCGAC CTTTGGTCGCCCGGCCTCAGTGAGCGAGCGAGCGCGCAGAGAGGGAGTGGCCAACT CCATCACTAGGGGTTCCT (SEQ ID NO: 487), and the 3′ ITR of the transgene of the AAV constructs of the disclosure has the sequence AGGAACCCCTAGTGATGGAGTTGGCCACTCCCTCTCTGCGCGCTCGCTCGCTCACTG AGGCCGGGCGACCAAAGGTCGCCCGACGCCCGGGCTTTGCCCGGGCGGCCTCAGTG AGCGAGCGAGCGCGCAGCTGCCTGCAGG (SEQ ID NO: 488). In other embodiments, the ITR sequences are modified to remove unmethylated CpG motifs to reduce immunogenic responses. In particular, CpG dinucleotide motifs (CpG PAMPs) in AAV vectors are immunostimulatory because of their high degree of hypomethylation, relative to mammalian CpG motifs, which have a high degree of methylation. In one embodiment, the modified AAV 2 ITR sequences are modified to remove CpG motifs, such that the 5′ITR has the sequence of TGCTCACTCACTCACTCACTGAGGCCTGCAGAGCAAAGCTCTGCAGTCTGGGGACCT TTGGTCCCCAGGCCTCAGTGAGTGAGTGAGTGAGCAGAGAGGGAGTGGCCAACTCC ATCACTAGGGGTTCCT (SEQ ID NO: 489) and the 3′ ITR sequence is the sequence TCTGCTCACTCACTCACTCACTGAGGCCTGCAGAGCAAAGCTCTGCAGTCTGGGGAC CTTTGGTCCCCAGGCCTCAGTGAGTGAGTGAGTGAGCAGAGAGGGAGTGGCCAACT CCATCACTAGGGGTTCCT of SEQ ID NO: 490. Similarly, the present disclosure provides rAAV vectors wherein one or more rAAV transgene component sequences selected from the group consisting of 5′ ITR, 3′ ITR, Pol III promoter, Pol II promoter, encoding sequence for CRISPR nuclease, encoding sequence for ERS, accessory element, and poly(A) are codon-optimized for depletion of all or a portion of the CpG dinucleotides, wherein the resulting rAAV vector transgene is substantially devoid of CpG dinucleotides. In some embodiments, the present disclosure provides rAAV vectors wherein one or more rAAV transgene component sequences selected from the group consisting of 5′ ITR, 3′ ITR, Pol III promoter, Pol II promoter, encoding sequence for a CRISPR nuclease, encoding sequence for ERS, 3′ UTR, poly(A) signal sequence, poly(A), and accessory element comprise less than about 10%, less than about 5%, or less than about 1% CpG dinucleotides. In some embodiments, the present disclosure provides rAAV vectors wherein one or more rAAV transgene component sequences selected from the group consisting of 5′ ITR, 3 ITR, Pol III promoter, Pol II promoter, encoding sequence for the CRISPR nuclease, encoding sequence for the ERS, 3′ UTR, poly(A) signal sequence, and poly(A) are devoid of CpG dinucleotides. In some embodiments, the present disclosure provides rAAV vectors wherein the transgene comprises less than about 10%, less than about 5%, or less than about 1% CpG dinucleotides. In some embodiments, the present disclosure provides rAAV vectors wherein the one or more rAAV component sequences codon-optimized for depletion of CpG dinucleotides are selected from the group of sequences consisting of SEQ ID NOS: 489, 490, 535-556, 559-564, and 49850-49861 as set forth in Tables 37, 38, and 51 or a sequence having at least about 80%, at least about 90%, at least about 95%, at least about 96%, at least about 97%, at least about 98%, or at least about 99% sequence identity thereto, wherein the resulting AAV exhibits a lower potential for inducing an immune response, either in vivo (when administered to a subject) or in in vitro mammalian cell assays designed to detect markers of an inflammatory response, wherein the reduced response is determined by the measurement of one or more parameters such as production of antibodies or a delayed-type hypersensitivity to an rAAV component, or the production of inflammatory cytokines and markers, such as, but not limited to TLR9, interleukin-1 (IL-1), IL-6, IL-12, IL-18, tumor necrosis factor alpha (TNF-α), interferon gamma (IFN-γ), and granulocyte-macrophage colony stimulating factor (GM-CSF).

By “AAV rep coding region” is meant the region of the AAV genome which encodes the replication proteins Rep 78, Rep 68, Rep 52 and Rep 40. These Rep expression products have been shown to possess many functions, including recognition, binding and nicking of the AAV origin of DNA replication, DNA helicase activity and modulation of transcription from AAV (or other heterologous) promoters. The Rep expression products are collectively required for replicating the AAV genome.

By “AAV cap coding region” is meant the region of the AAV genome which encodes the capsid proteins VP1, VP2, and VP3, or functional homologues thereof. These Cap expression products supply the packaging functions which are collectively required for packaging the viral genome.

In some embodiments, AAV capsids utilized for delivery of the nucleic acids encoding the engineered CasX, ERS, and, optionally, donor template nucleotides, to a host cell can be derived from any of several AAV serotypes, including without limitation, AAV1, AAV2, AAV3, AAV4, AAV5, AAV6, AAV7, AAV8, AAV9, AAV10, AAV11, AAV12, AAV 9.45, AAV 9.61, AAV 44.9, AAV-Rh74 (Rhesus macaque-derived AAV), and AAVRh10. In some embodiments, the AAV vector and the regulatory sequences are selected so that the total size of the vector is about 4.7 to 5 kb or less, permitting packaging within the AAV capsid. While the AAV vector may be of any AAV serotype, nervous cell tropism varies among AAV capsid serotypes. Thus, use of AAV serotypes compatible with widespread transgene delivery to astrocytes and motoneurons is preferred. In some embodiments, the AAV vector is of serotype 9 or of serotype 6, which have been demonstrated to effectively deliver polynucleotides to motor neurons and glia throughout the spinal cord in preclinical models of ALS (Foust, K D. et al. Therapeutic AAV9-mediated suppression of mutant SOD1 slows disease progression and extends survival in models of inherited ALS. Mol Ther. 21(12):2148 (2013)). In some embodiments, the methods provide use of AAV9 or AAV6 for targeting of neurons via intraparenchymal brain injection. In some embodiments, the methods provide use of AAV9 for intravenous administering of the vector wherein the AAV9 has the ability to penetrate the blood-brain barrier and drive gene expression in the nervous system via both neuronal and glial tropism of the vector. In other embodiments, the AAV vector is derived from serotype 8, which has been demonstrated to effectively deliver polynucleotides to neurons, liver, skeletal muscle and the heart. In other embodiments, the AAV vector is derived from serotype 5, which has been demonstrated to effectively deliver polynucleotides to neurons. In other embodiments, the AAV vector is derived from AAV serotype 2, which has been demonstrated to effectively deliver polynucleotides to retinal cells, skeletal muscle, neurons, vascular smooth muscle cells, and hepatocytes.

In order to eliminate any integrative capacity of the virus, recombinant AAV vectors remove rep and cap from the DNA of the viral genome and a three plasmid system can be utilized to transfect a suitable host packaging cell. To produce such vectors, the desired transgenes, together with promoters to drive transcription of the transgenes and any enhancer elements, are inserted between the ITRs, and the rep and cap genes are provided in trans in a second plasmid. A third plasmid, providing helper genes such as adenovirus E4, E2a and VA genes, is also used. All three plasmids are then transfected into an appropriate packaging cell using known techniques, such as by transfection. Alternatively, the host cell genome may comprise stably integrated Rep and Cap genes. Suitable packaging cell lines are known to one of ordinary skill in the art. See for example, www.cellbiolabs.com/aav-expression-and-packaging.

In an advantage of rAAV constructs of the present disclosure, the smaller size of the CRISPR Type V nucleases; e.g., the engineered CasX of the embodiments, permits the inclusion of all the necessary editing and ancillary expression components into the transgene such that a single rAAV particle can deliver and transduce these components into a target cell in a form that results in the expression of the CRISPR nuclease and ERS that are capable of effectively modifying the target nucleic acid of the target cell. This stands in marked contrast to other CRISPR systems, such as Cas9, where typically a two-particle system is employed to deliver the necessary editing components to a target cells.

Thus, in some embodiments of the rAAV systems, the disclosure provides; i) a first plasmid comprising the ITRs, sequences encoding the engineered CasX, sequences encoding one or more ERS, a first promoter operably linked to the CasX and a second promoter operably linked to the ERS, and, optionally, a 3′ UTR, a poly(A) signal sequence, a poly(A) sequence, and one or more enhancer elements; ii) a second plasmid comprising the rep and cap genes; and iii) a third plasmid comprising helper genes, wherein upon transfection of an appropriate packaging cell, the cell is capable of producing an rAAV having the ability to deliver to a target cell, in a single particle, sequences capable of expressing the engineered CasX nuclease and ERS having the ability to edit the target nucleic acid of the target cell. In some embodiments of the rAAV systems, the sequence encoding the CRISPR protein and the sequence encoding the at least first ERS are less than about 3100, less than about 3090, less than about 3080, less than about 3070, less than about 3060, less than about 3050, or less than about 3040 nucleotides in length, such that the sequences encoding the first and second promoter and, optionally, one or more enhance elements can have at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In some embodiments of the rAAV systems, the sequence encoding the first promoter and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. In some embodiments of the rAAV systems, the sequence encoding the first and second promoters and the at least one accessory element have greater than at least about 1300, at least about 1350, at least about 1360, at least about 1370, at least about 1380, at least about 1390, at least about 1400, at least about 1500, at least about 1600 nucleotides, at least 1650, at least about 1700, at least about 1750, at least about 1800, at least about 1850, or at least about 1900 nucleotides in combined length. Non-limiting examples of such rAAV systems and encoding sequences are disclosed in the Examples, below.

Packaging cells are typically used to form virus particles. The eukaryotic host packaging cell can be selected from Baby Hamster Kidney fibroblast (BHK) cells, human embryonic kidney 293 (HEK293), human embryonic kidney 293T (HEK293T), NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS), HeLa, Chinese hamster ovary (CHO) cells, or other eukaryotic cells known in the art suitable for the production of recombinant AAV. A number of transfection techniques are generally known in the art; see, e.g., Sambrook et al. (1989) Molecular Cloning, a laboratory manual, Cold Spring Harbor Laboratories, New York. Particularly suitable transfection methods include calcium phosphate co-precipitation, direct microinjection into cultured cells, electroporation, liposome mediated gene transfer, lipid-mediated transduction, and nucleic acid delivery using high-velocity microprojectiles.

In some embodiments, host cells transfected with the above-described AAV expression vectors are rendered capable of providing AAV helper functions in order to replicate and encapsidate the nucleotide sequences flanked by the AAV ITRs to produce rAAV viral particles. AAV helper functions are generally AAV-derived coding sequences which can be expressed to provide AAV gene products that, in turn, function in trans for productive AAV replication. AAV helper functions are used herein to complement necessary AAV functions that are missing from the AAV expression vectors. Thus, AAV helper functions include one, or both of the major AAV ORFs (open reading frames), encoding the rep and cap coding regions, or functional homologues thereof. Accessory functions can be introduced into and then expressed in host cells using methods known to those of skill in the art.

In other embodiments, suitable vectors may include XDP. XDP particles are particles that closely resemble viruses, but do not contain viral genetic material and are therefore non-infectious. In some embodiments, the disclosure provides XDPs produced in vitro that comprise an eCasX:ERS RNP complex. Non-limiting, exemplary XDP systems are described in PCT/US20/63488 and WO2021113772A1, incorporated by reference herein. In some embodiments, the disclosure provides host cells comprising polynucleotides or vectors encoding any of the foregoing XDP embodiments. Combinations of structural proteins from different viruses can be used to create XDPs, including components from virus families including Parvoviridae (e.g., adeno-associated virus), Retroviridae (e.g., HIV and Alpharetrovirus), Flaviviridae (e.g., Hepatitis C virus), Paramyxoviridae (e.g., Nipah) and bacteriophages (e.g., Qβ, AP205). In some embodiments, the disclosure provides XDP systems designed using components of retrovirus, including lentiviruses such as HIV, Alpharetrovirus, and other genera of the Retroviridae, in which individual plasmids comprising polynucleotides encoding the various components are introduced into a packaging cell that, in turn, produce the XDP. In some embodiments, the disclosure provides XDP comprising polynucleotides encoding one or more components of i) protease, ii) a protease cleavage site, iii) a Gag polyprotein or one or more components of a Gag polyprotein selected from matrix protein (MA), nucleocapsid protein (NC), capsid protein (CA), or p1-p6 protein, iv) a Gag-pol polyprotein or a truncated version lacking reverse transcriptase (RT) and integrase but comprising HIV protease (Gag-TFR-PR), v) engineered CasX; vi) ERS, and vi) targeting glycoproteins or antibody fragments wherein the resulting XDP particle encapsidates multiple eCasX:ERS RNPs. The polynucleotides encoding the Gag, engineered CasX and ERS can further comprise paired components designed to assist the trafficking of the components out of the nucleus of the host cell and into the budding XDP. Non-limiting examples of such trafficking components include hairpin RNA such as MS2 hairpin, PP7 hairpin, Qβ hairpin, and U1 hairpin II that have binding affinity for MS2 coat protein, PP7 coat protein, Qβ coat protein, and U1A signal recognition particle, respectively. In other embodiments, the ERS can comprise Rev response element (RRE) or portions thereof that have binding affinity to Rev, which can be linked to the Gag polyprotein.

The targeting glycoproteins or antibody fragments on the surface that provides tropism of the XDP to the target cell, wherein upon administration and entry into the target cell, the RNP molecule is free to be transported into the nucleus of the cell. In other embodiments, the disclosure provides XDP of the foregoing and further comprises a second ERS or a donor template. The foregoing offers advantages over other vectors in the art in that viral transduction to dividing and non-dividing cells is efficient and that the XDP delivers potent and short-lived RNP that escape a subject's immune surveillance mechanisms that would otherwise detect a foreign protein. The disclosure contemplates multiple configurations of the arrangement of the encoded components, including duplicates of some of the encoded components. The envelope glycoprotein can be derived from any enveloped viruses known in the art to confer tropism to XDP, including but not limited to the group consisting of Argentine hemorrhagic fever virus, Australian bat virus, Autographa californica multiple nucleopolyhedrovirus, Avian leukosis virus, baboon endogenous virus, Bolivian hemorrhagic fever virus, Boma disease virus, Breda virus, Bunyamwera virus, Chandipura virus, Chikungunya virus, Crimean-Congo hemorrhagic fever virus, Dengue fever virus, Duvenhage virus, Eastern equine encephalitis virus, Ebola hemorrhagic fever virus, Ebola Zaire virus, enteric adenovirus, Ephemerovirus, Epstein-Bar virus (EBV), European bat virus 1, European bat virus 2, Fug Synthetic gP Fusion, Gibbon ape leukemia virus, Hantavirus, Hendra virus, hepatitis A virus, hepatitis B virus, hepatitis C virus, hepatitis D virus, hepatitis E virus, hepatitis G Virus (GB virus C), herpes simplex virus type 1, herpes simplex virus type 2, human cytomegalovirus (HHV5), human foamy virus, human herpesvirus (HHV), human Herpesvirus 7, human herpesvirus type 6, human herpesvirus type 8, human immunodeficiency virus 1 (HIV-1), human metapneumovirus, human T-lymphotropic virus 1, influenza A, influenza B, influenza C virus, Japanese encephalitis virus, Kaposi's sarcoma-associated herpesvirus (HHV8), Kaysanur Forest disease virus, La Crosse virus, Lagos bat virus, Lassa fever virus, lymphocytic choriomeningitis virus (LCMV), Machupo virus, Marburg hemorrhagic fever virus, measles virus, Middle eastern respiratory syndrome-related coronavirus, Mokola virus, Moloney murine leukemia virus, monkey pox, mouse mammary tumor virus, mumps virus, murine gammaherpesvirus, Newcastle disease virus, Nipah virus, Nipah virus, Norwalk virus, Omsk hemorrhagic fever virus, papilloma virus, parvovirus, pseudorabies virus, Quaranfil virus, rabies virus, RD 114 Endogenous Feline Retrovirus, respiratory syncytial virus (RSV), Rift Valley fever virus, Ross River virus, rRotavirus, Rous sarcoma virus, rubella virus, Sabia-associated hemorrhagic fever virus, SARS-associated coronavirus (SARS-CoV), Sendai virus, Tacaribe virus, Thogotovirus, tick-borne encephalitis causing virus, varicella zoster virus (HHV3), varicella zoster virus (HHV3), variola major virus, variola minor virus, Venezuelan equine encephalitis virus, Venezuelan hemorrhagic fever virus, vesicular stomatitis virus (VSV), VSV-G, Vesiculovirus, West Nile virus, western equine encephalitis virus, and Zika Virus.

Upon production and recovery of the XDP comprising the eCasX:ERS RNP of any of the embodiments described herein, the XDP can be used in methods to edit target cells of subjects by the administering of such XDP, as described more fully, below.

For non-viral delivery, vectors can also be delivered wherein the vector or vectors encoding the engineered CasX and ERS are formulated in nanoparticles, wherein the nanoparticles contemplated include, but are not limited to nanospheres, liposomes, lipid nanoparticles (LNP), quantum dots, polyethylene glycol particles, hydrogels, and micelles. n some embodiments, the engineered CasX and ERS of the embodiments disclosed herein are formulated in a lipid nanoparticle, described more fully, below.

### VII. Methods for Modification of a Target Nucleic Acid

The engineered CasX proteins, ERS, nucleic acids, and variants thereof provided herein, as well as vectors encoding such components, are useful for various applications, including therapeutics, diagnostics, and research. To effect the methods of the disclosure for gene editing, resulting in modification of the gene, provided herein are programmable systems comprising the engineered CasX proteins and ERS. The programmable nature of the systems provided herein allows for the precise targeting to achieve the desired effect (nicking, cleaving, repairing, etc.) at one or more regions of predetermined interest in the target nucleic acid sequence of the target gene.

A variety of strategies and methods can be employed to modify the target nucleic acid sequence in a cell using the systems provided herein. As described herein, an engineered CasX introducing double-stranded cleavage of the target nucleic acid generates a double-stranded break within 18-26 nucleotides 5′ of a PAM site on the target strand and 10-18 nucleotides 3′ on the non-target strand. The resulting modification can result in random insertions or deletions (indels), or a substitution, duplication, frame-shift, or inversion of one or more nucleotides in those regions by non-homologous DNA end joining (NHEJ) repair mechanisms. Alternatively, the editing event may be a cleavage event followed by homology-directed repair (HDR), homology-independent targeted integration (HITI), micro-homology mediated end joining (MMEJ), single strand annealing (SSA) or base excision repair (BER), resulting in modification of the target nucleic acid sequence. In some embodiments of the method, the modification comprises introducing an in-frame mutation in the target nucleic acid. In some embodiments of the method, the modification comprises introducing a frame-shifting mutation in the target nucleic acid. In some embodiments of the method, the modification comprises introducing a premature stop codon in the coding sequence in the target nucleic acid. As a result of a gene knock-down by the foregoing modifications, the protein activity or function may be attenuated or the protein levels may be reduced or eliminated. In some embodiments of the method, the modification results in at least about 10%, at least about 20%, at least about 30%, at least about 40%, at least about 50%, at least about 60%, at least about 70%, at least about 80%, or at least about 90% or more reduced expression of the gene product in the modified cells of the population in comparison to cells in which the gene has not been modified. In other embodiments, the disclosure provides systems and methods for correcting mutations in the gene wherein a corrective sequence is knocked-in by introducing mutations at select locations by design of the targeting sequence linked to the ERS such that a wild-type or functional gene product is expressed.

In some embodiments, the disclosure provides methods of modifying a target nucleic acid in a cell, the method comprising contacting the target nucleic acid of the cell with: i) an engineered CasX protein and ERS editing pair comprising an engineered CasX and an ERS of any one of the embodiments described herein; ii) a nucleic acid encoding the engineered CasX and the ERS editing pair; iii) a vector comprising the nucleic acid of (ii), above; iv) an XDP comprising the eCasX:ERS editing pair of any one of the embodiments described herein; v) an LNP comprising an ERS and a nucleic acid encoding the engineered CasX; or vi) combinations of two or more of (i) to (v), wherein the contacting of the target nucleic acid with an engineered CasX protein and ERS gene editing pair and, optionally, the donor template, modifies the target nucleic acid in the cell. In some cases, the modification results in a correction or compensation of a mutation in a cell, thereby creating an edited cell such that expression of a functional gene product can occur. In other embodiments of the method, the modification comprises reducing or eliminating expression of the gene product by a knock-down or knock-out of the gene.

In some embodiments of the method of modifying a target nucleic acid sequence in a cell, wherein the method comprises contacting the target nucleic acid of the cell with an editing pair, wherein the editing pair comprises an engineered CasX selected from the group consisting of the sequences of SEQ ID NOS: 247-294, 24916-49628, 49746-49747, and 49871-49873, or a variant sequence at least 60% identical, at least 70% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, or at least 99.5% identical thereto, the ERS scaffold comprises a sequence selected from the group consisting of the sequences of SEQ ID NOS: 156, 739-907, 11568-22227, 23572-24915, 49719-49735, and 49871-49873, or a sequence at least 65% identical, at least 70% identical, at least 75% identical, at least 80% identical, at least 81% identical, at least 82% identical, at least 83% identical, at least 84% identical, at least 85% identical, at least 86% identical, at least 86% identical, at least 87% identical, at least 88% identical, at least 89% identical, at least 89% identical, at least 90% identical, at least 91% identical, at least 92% identical, at least 93% identical, at least 94% identical, at least 95% identical, at least 96% identical, at least 97% identical, at least 98% identical, at least 99% identical, at least 99.5% identical thereto, and the ERS comprises a targeting sequence that is complementary to the target nucleic acid and is capable of hybridizing with the target nucleic acid.

In those cases where the engineered CasX is delivered to the cell in the protein form and the ERS is delivered in the RNA form, the engineered CasX and ERS can be pre-complexed and delivered as an RNP. In those cases where the engineered CasX and ERS are delivered to the target cell as nucleic acids and then expressed in the cell, the engineered CasX and ERS can associate as an RNP. In those cases where an LNP delivers the ERS and the engineered CasX is delivered as an mRNA and then is expressed in the cell, the engineered CasX and ERS can associate as an RNP. In the foregoing, the engineered CasX protein provides the site-specific activity and is guided to a target site (and further stabilized at a target site) within a target nucleic acid sequence to be modified by virtue of its association with the ERS. The engineered CasX protein of the RNP complex provides the site-specific activities of the complex such as binding, introducing a single-strand break or a double-strand break within or near the gene that results in a modification of the target nucleic acid such as a permanent indel (deletion or insertion) or other mutation (a base change, inversion or rearrangement with respect to the genomic sequence) in the target nucleic acid, as described herein, with a corresponding modulation of expression or alteration in the function of the gene product, thereby creating a modified cell.

In other embodiments of the method of modifying a target nucleic acid sequence in a cell, the method comprises contacting the target nucleic acid sequence with a plurality of RNPs with a first and a second, or prwith three, or with four or more ERSs targeted to different or overlapping portions of the gene wherein the engineered CasX protein introduces multiple breaks, either single-stranded or double-stranded, in the target nucleic acid that result in permanent indels (introducing an insertion, or a deletion) or mutations in the target nucleic acid, as described herein, or an excision of the intervening sequence between the breaks with a corresponding modulation of expression or alteration in the function of the gene product, thereby creating a modified cell.

In other embodiments, the disclosure provides methods of modifying a target nucleic acid sequence of a cell, comprising contacting said cell with a vector of any of the embodiments described herein comprising a nucleic acid encoding a eCasX:ERS gene editing pair comprising an engineered CasX protein and an ERS of any of the embodiments described herein and, optionally, a donor template, wherein the ERS comprises a targeting sequence complementary to, and therefore capable of hybridizing with, the target nucleic acid sequence, wherein the contacting results in modification of the target nucleic acid. Introducing recombinant expression vectors into cells in vitro can occur in any suitable culture media and under any suitable culture conditions that promote the survival of the cells. Introducing recombinant expression vectors into a target cell can be carried out in vivo by administration to a subject using methods and regimens described below.

In some embodiments, vectors may be provided directly to a target host cell. For example, cells may be contacted with vectors comprising the subject nucleic acids (e.g., recombinant expression vectors encoding the ERS and the engineered CasX protein) such that the vectors are taken up by the cells. Methods for contacting cells with nucleic acid vectors that are plasmids include electroporation, calcium chloride transfection, microinjection, and lipofection are well known in the art. For viral vector delivery, cells can be contacted with viral particles comprising the subject viral expression vectors; e.g., the vectors are viral particles such as AAV or VLP that comprise polynucleotides that encode the eCasX:ERS components. For non-viral delivery, vectors or the eCasX:ERS components can also be formulated for delivery in lipid nanoparticles, described more fully, below.

In some embodiments, the modifying of the target nucleic acid occurs in vitro, inside of a cell, for example in a cell culture system. In some embodiments, the modifying occurs in vivo inside of a cell of a subject, for example in a cell in an animal. In some embodiments, the cell is a eukaryotic cell. Exemplary eukaryotic cells may include cells selected from the group consisting of a mouse cell, a rat cell, a pig cell, a dog cell, and a non-human primate cell. In some embodiments, the cell is a human cell. Non-limiting examples of cells include an embryonic stem cell, an induced pluripotent stem cell, a germ cell, a fibroblast, an oligodendrocyte, a glial cell, a hematopoietic stem cell, a neuron progenitor cell, a neuron, a muscle cell, a bone cell, a hepatocyte, a pancreatic cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, a parenchymal cell, an epithelial cell, an endothelial cell, a mesothelial cell, fibroblasts, osteoblasts, chondrocytes, exogenous cell, endogenous cell, stem cell, hematopoietic stem cell, bone-marrow derived progenitor cell, myocardial cell, skeletal cell, fetal cell, undifferentiated cell, multi-potent progenitor cell, unipotent progenitor cell, a monocyte, a cardiac myoblast, a skeletal myoblast, a macrophage, a capillary endothelial cell, a xenogenic cell, an allogenic cell, or a post-natal stem cell. In alternative embodiments, the cell is a prokaryotic cell.

In some embodiments of the methods of modifying a target nucleic acid of a cell in vitro or ex vivo, to induce cleavage or any desired modification to a target nucleic acid, the ERS and the engineered CasX protein of the present disclosure and, optionally, the donor template sequence, whether they be introduced as nucleic acids or polypeptides, complexed RNP, vectors or XDP, are provided to the cells for about 30 minutes to about 24 hours, or at least about 1 hour, 1.5 hours, 2 hours, 2.5 hours, 3 hours, 3.5 hours 4 hours, 5 hours, 6 hours, 7 hours, 8 hours, 12 hours, 16 hours, 18 hours, 20 hours, or any other period from about 30 minutes to about 24 hours, which may be repeated with a frequency of about every day to about every 4 days, e.g., every 1.5 days, every 2 days, every 3 days, or any other frequency from about every day to about every four days. The agent(s) may be provided to the subject cells one or more times, e.g., one time, twice, three times, or more than three times, and the cells allowed to incubate with the agent(s) for some amount of time following each contacting event; e.g., 30 minutes to about 24 hours. In the case of in vitro-based methods, after the incubation period with the engineered CasX and ERS (and optionally the donor template), the media is replaced with fresh media and the cells are cultured further.

In some embodiments, the method comprises administering to a subject a therapeutically-effective dose of a population of cells modified to correct or compensate for the mutation of the gene. In some embodiments, the administration of the modified cells results in the expression of wild-type or a functional gene product in the subject. In one embodiment, the cells are autologous with respect to the subject to be administered the cells. In another embodiment, the cells are allogeneic with respect to the subject to be administered the cells. In some cases, the subject is selected from the group consisting of mouse, rat, pig, and non-human primate. In other cases, the subject is a human.

### VIII. Therapeutic Methods

In another aspect, the present disclosure relates to methods of treating a disease or disorder in a subject in need thereof. A number of therapeutic strategies have been used to design the systems for use in the methods of treatment of a subject with a disease or disorder related to a genetic mutation. In some embodiments, the modification of the target nucleic acid occurs in a subject having a mutation in an allele of a gene wherein the mutation causes a disease or disorder in the subject. In some embodiments, the modification of the target nucleic acid changes the mutation to a wild type allele of the gene or results in the expression of a functional gene product. In some embodiments, the modification of the target nucleic acid knocks down or knocks out expression of an allele of a gene causing a disease or disorder in the subject.

In some embodiments, the method comprises administering to the subject a therapeutically effective dose of a system comprising a gene editing pair of an engineered CasX and ERS disclosed herein with a linked targeting sequence complementary to the target nucleic acid to be modified. In some embodiments, the method of treatment comprises administering to the subject a therapeutically effective dose of: i) a eCasX:ERS system comprising ant engineered CasX and a first ERS (with a targeting sequence complementary to the target nucleic acid to be modified) of any of the embodiments described herein; ii) a nucleic acid encoding the eCasX:ERS system of (i); iii) a vector comprising the nucleic acid of (ii), which can be an AAV of any of the embodiments described herein; iv) a XDP comprising the eCasX:ERS system of (i); v) an LNP comprising an ERS and a nucleic acid encoding the engineered CasX; or vi) combinations of two or more of (i)-(v), wherein 1) the gene of the cells of the subject targeted by the first ERS is modified (e.g., knocked-down or knocked-out) by the engineered CasX protein (and, optionally, the donor template); or 2) the gene of the cells of the subject targeted by the first ERS is corrected or modified by the engineered CasX protein such that a functional gene product can be expressed. In some embodiments, the method of treating further comprises administering a second, third, or fourth ERS or nucleic acids encoding the ERS, or an XDP comprising a second, third, or fourth ERS, wherein the second, third, or fourth ERS have targeting sequences complementary to a different or overlapping portion of the target nucleic acid sequence compared to the first ERS. In some cases, the use of a second ERS complexed with an engineered CasX results in edits to a different gene than the first ERS. In other cases, the use of a second ERS targeting the same gene as the first ERS can result in the excision of the nucleotides between the two cleavage locations. It will be understood that in the foregoing, each different ERS is paired with an engineered CasX protein. In embodiments in which two or more gene editing pairs are provided to the cell (e.g., comprising two ERS comprising two or more different spacers that are complementary to different sequences within the same or different target nucleic acid), the gene pairs may be provided simultaneously in the same vector (e.g., as two RNPS and/or within a single AAV vector), or delivered simultaneously in separate vectors. Alternatively, they may be provided consecutively, e.g., the first gene editing pair being provided first, followed by the second gene editing pair, or vice versa.

In some embodiments, method of treatment comprises administering a therapeutically effective dose of an AAV vector encoding the eCasX:ERS system, wherein the capsid of the AAV vector is selected from AAV1, AAV2, AAV3, AAV4, AAV5, AAV6, AAV7, AAV8, AAV9, AAV10, AAV 9.45, AAV 9.61, AAV-Rh74, or AAVRh10. In other embodiments, the method of treatment comprises administering a therapeutically effective dose of a XDP comprising RNP of the eCasX:ERS system to the subject. In other embodiments, the method of treatment comprises administering a therapeutically effective dose of an LNP comprising an ERS and a nucleic acid encoding the engineered CasX. The vector, XDP, or LNP can be administered by a route of administration selected from the group consisting of intraparenchymal, intravenous, intra-arterial, intramuscular, subcutaneous, intracerebroventricular, intracisternal, intrathecal, intracranial, intravitreal, subretinal, intracapsular, and intraperitoneal routes or combinations thereof, wherein the administering method is injection, transfusion, or implantation. The administration can be once, twice, or can be administered multiple times using a regimen schedule of weekly, every two weeks, monthly, quarterly, every six months, once a year, or every 2 or 3 years. In some cases, the subject is selected from the group consisting of mouse, rat, pig, and non-human primate. In other cases, the subject is a human.

In some embodiments of the method, the modifying comprises introducing a single-stranded break in the target nucleic acid of the targeted cells of a subject. In other cases, the modifying comprises introducing a double-stranded break in the target nucleic acid of the targeted cells of a subject. In some embodiments, the modifying introduces one or more mutations in the target nucleic acid, such as an insertion, deletion, substitution, duplication, or inversion of one or more nucleotides in the gene, wherein expression of the gene product in the modified cells of the subject is reduced by at least about 10%, at least about 20%, at least about 30%, at least about 40%, at least about 50%, at least about 60%, at least about 70%, at least about 80%, or at least about 90% or more in comparison to a cell that has not been modified. In some cases, the gene of the modified cells of the subject are modified such that least 70%, at least 75%, at least 80%, at least 85%, at least 90%, or at least 95% of the modified cells do not express a detectable level of the gene product. In some embodiments, the administering of the therapeutically effective amount of an eCasX:ERS system to knock down or knock out expression of a gene product to a subject with a disease leads to the prevention or amelioration of the underlying disease such that an improvement is observed in the subject, notwithstanding that the subject may still be afflicted with the underlying disease. In some embodiments, the administering of the therapeutically effective amount of a eCasX:ERS system to correct or compensate for a mutation a gene product to a subject with a disease leads to the prevention or amelioration of the underlying disease such that an improvement is observed in the subject, notwithstanding that the subject may still be afflicted with the underlying disease. In such embodiments, the gene can be modified by the NHEJ host repair mechanisms, or utilized in conjunction with a donor template that is inserted by HDR or HITI mechanisms to either excise, correct, or compensate for the mutation in the cells of the subject, such that expression of a wild-type or functional gene product in modified cells is increased by at least about 50%, at least about 60%, at least about 70%, at least about 80%, at least about 90%, or at least about 95% in comparison to a cell that has not been modified. In some embodiments, the administration of the therapeutically effective amount of the engineered CasX and ERS system leads to an improvement in at least one clinically-relevant parameter for a disease.

IX. Particles for Delivery of the eCasX:ERS Systems

In another aspect, the present disclosure provides particle compositions for delivery of the repressor systems, such as the eCasx:ERS systems described herein, to cells or to subjects for the repression of a gene. Particles envisaged as within the scope of the instant disclosure include, but are not limited to, nanoparticles such as synthetic nanoparticles, polymeric nanoparticles, lipid nanoparticles, viral particles and virus-like particles. Particles of the disclosure may encapsulate payloads such as ERS variants, as described herein, optionally in combination with mRNA encoding the engineered CasX proteins of any of the embodiments described herein. Alternatively, or in addition, particles of the disclosure may encapsulate payloads of ERS variants and engineered CasX proteins, for example when associated as a ribonucleoprotein (RNP) complex. In some embodiments, the particles are synthetic nanoparticles that encapsulate payloads of ERS variants and mRNA encoding engineered CasX of any of the embodiments described herein. In some embodiments, the synthetic nanoparticles comprise biodegradable polymeric nanoparticles (PNP). In some embodiments, materials for the creation of biodegradable polymeric nanoparticles (PNP) include polylactide, poly (lactic-co-glycolic acid) (PLGA), poly(ethyl cyanoacrylate), poly(butyl cyanoacrylate), poly(isobutyl cyanoacrylate), and poly(isohexyl cyanoacrylate), polyglutamic acid (PGA), poly (ε-caprolactone) (PCL), cyclodextrin, and natural polymers for instance chitosan, albumin, gelatin, and alginate, which are the most utilized polymers for the synthesis of PNP (Production and clinical development of nanoparticles for gene delivery. Molecular Therapy-Methods & Clinical Development 3:16023; doi:10.1038 (2016)). In other embodiments, the particles are lipid nanoparticles that encapsulate ERS variants and mRNA encoding engineered CasX of any of the embodiments described herein, described more fully, below.

a. Lipid Nanoparticles (LNP)

The present disclosure provides lipid nanoparticles (LNP) for delivery of the eCasX:ERS systems described herein to cells or to subjects for the repression of a gene. In some embodiments, the LNPs of the disclosure are tissue-specific, have excellent biocompatibility, and can deliver the eCasX:ERS systems with high efficiency, and thus can be used for the repression of the targeted gene.

The disclosure further provides LNP compositions and pharmaceutical compositions comprising a plurality of the LNP described herein.

In their native forms, nucleic acid polymers are unstable in biological fluids and cannot penetrate into the cytoplasm of target cells, thus requiring delivery systems. Lipid nanoparticles (LNP) have proven useful for both the protection and delivery of nucleic acids to tissues and cells. Furthermore, the use of mRNA in LNPs to encode the engineered CasX eliminates the possibility of undesirable genome integration, as compared to DNA vectors. Moreover, mRNA efficiently translates into protein in both mitotic and non-mitotic cells, as it does not require entry into the nucleus since it exerts its function in the cytoplasmic compartment. LNPs as a delivery platform thus offer the additional advantage of being able to co-formulate both the mRNA encoding the CRISPR nuclease and the ERS into single LNP particles.

Accordingly, in various embodiments, the disclosure encompasses lipid nanoparticles and compositions that may be used for a variety of purposes, including the delivery of encapsulated or associated (e.g., complexed) therapeutic agents such as nucleic acids to cells, both in vitro and in vivo. In certain embodiments, the disclosure encompasses methods of treating or preventing diseases or disorders in a subject in need thereof by contacting the subject with a lipid nanoparticle that encapsulates or is associated with a suitable therapeutic agent complexed through various physical, chemical or electrostatic interactions between one or more of the lipid components used in the compositions to make LNPs. In some embodiments, the suitable therapeutic agent comprises a eCasX:ERS system as described herein.

In certain embodiments, the lipid nanoparticles are useful for the delivery of nucleic acids, including, e.g., the mRNA encoding the engineered CasX of the disclosure, including the sequences of SEQ ID NOS: 247-294, 24916-49628, 49746-49747, and 49871-49873, and the ERS variants of the disclosure, including the sequences of SEQ ID NOS: 156, 739-907, 11568-22227, 23572-24915, and 49719-49735. In some embodiments, the present disclosure provides LNP in which the ERS and mRNA encoding the engineered CasX are incorporated into single LNP particles. In other embodiments, the present disclosure provides LNP in which the ERS and mRNA encoding the engineered CasX are incorporated into separate populations of LNPs, which can be formulated together in varying ratios for administration.

The lipid nanoparticles and lipid nanoparticle compositions of certain embodiments of the disclosure may be used to repress expression of a desired protein both in vitro and in vivo by contacting cells with a lipid nanoparticle comprising one or more ionizable lipids described herein, wherein the lipid nanoparticle encapsulates or is associated with a nucleic acid that is expressed to produce the desired protein (e.g., a messenger RNA encoding the engineered CasX protein). In some embodiments, the lipid nanoparticles and compositions may be used to repress the expression of a target gene both in vitro and in vivo by contacting cells with a lipid nanoparticle comprising one or more novel ionizable cationic lipids or permanently charged cationic lipids described herein, wherein the lipid nanoparticle encapsulates or is associated with one or more nucleic acids of the eCasX:ERS systems of the disclosure that repress the targeted gene. The lipid nanoparticles and compositions of embodiments of the disclosure may also be used for co-delivery of different nucleic acids (e.g., mRNA, gRNA, siRNA, saRNA, mcDNA, and plasmid DNA) separately or in combination, such as may be useful to provide an effect requiring colocalization of different nucleic acids (e.g., mRNA encoding for a suitable gene repressing factor or enzyme and ERS for targeting of the gene).

In some embodiments, LNPs and LNP compositions described herein include at least one cationic lipid, at least one conjugated lipid, at least one steroid or derivative thereof, at least one helper lipid, or any combination thereof. Alternatively, the lipid compositions of the disclosure can include an ionizable lipid, such as an ionizable cationic lipid, a helper lipid (usually a phospholipid), cholesterol, and a polyethylene glycol-lipid conjugate (PEG-lipid) to improve the colloidal stability in biological environments by, for example, reducing a specific absorption of plasma proteins and forming a hydration layer over the nanoparticles. Such lipid compositions can be formulated at typical mole ratios of 50:10:37-39:13 or 20-50:8-65:15-70:1-3.0 of IL:HL:Sterol: PEG-lipid, with variations made to adjust individual properties.

The LNPs and LNP compositions of the present disclosure are configured to protect and deliver an encapsulated payload of the systems of the disclosure to tissues and cells, both in vitro and in vivo. Various embodiments of the LNPs and LNP compositions of the present disclosure are described in further detail herein.

b. Cationic Lipid

In some aspects, the LNPs and LNP compositions of the present disclosure include at least one cationic lipid. The term “cationic lipid,” refers to a lipid species that has a net positive charge. In some embodiments, the cationic lipid is an ionizable cationic lipid that has a net positive charge at a selected pH<pKa of the ionizable lipid. In some embodiments, the ionizable cationic lipid has a pKa less than 7 such that the LNPs and LNP compositions achieve efficient encapsulation of the payload at a relatively low pH below the pKa of the respective lipid. In some embodiments, the cationic lipid has a pKa of about 5 to about 8, about 5.5 to about 7.5, about 6 to about 7, or about 6.5 to about 7. In some embodiments, the cationic lipid may be protonated at a pH below the pKa of the cationic lipid, and it may be substantially neutral at a pH over the pKa. The LNPs and LNP compositions may be safely delivered to a target organ (for example, the liver, lung, heart, spleen, as well as to tumors) and/or cell(hepatocyte, LSEC, cardiac cell, cancer cell, etc.) in vivo, and during endocytosis, exhibit a positive charge when pH drops below the ionizable lipid pKa to release the encapsulated payload through electrostatic interaction with an anionic lipids of the endosomal membrane.

Early formulations of LNP utilizing permanently cationic lipids resulted in LNPs with positive surface charge that proved toxic in vivo, plus were rapidly cleared by phagocytic cells. By changing to ionizable cationic lipids bearing tertiary amines, especially those with pKa<7, results in LNP achieving efficient encapsulation of nucleic acid polymers at low pH by interacting electrostatically with the negative charges of the phosphate backbone of mRNA, that also result in largely neutral systems at physiological pH values, thus alleviating problems associated with permanently-charged cationic lipids.

As used herein, “ionizable lipid” means an amine-containing lipid which can be easily protonated, and, for example, it may be a lipid of which charge state changes depending on the surrounding pH. The ionizable lipid may be protonated (positively charged) at a pH below the pKa of a cationic lipid, and it may be substantially neutral at a pH over the pKa. In one example, the LNP may comprise a protonated ionizable lipid and/or an ionizable lipid showing neutrality. In some embodiments, the LNP has a pKa of 5 to 8, 5.5 to 7.5, 6 to 7, or 6.5 to 7. The pKa of the LNP is important for in vivo stability and release of the nucleic acid payload of the LNP in the target cell or organ. In some embodiments, the LNP having the foregoing pKa ranges may be safely delivered to a target organ (for example, the liver, lung, heart, spleen, as well as to tumors) and/or target cell (hepatocyte, LSEC, cardiac cell, cancer cell, etc.) in vivo, and inside the endosome, exhibit a positive charge to release the encapsulated payload through electrostatic interaction with an anionic lipids of the endosomal membrane.

The ionizable lipid is an ionizable compound having characteristics similar to lipids generally, and through electrostatic interaction with a nucleic acid (for example, an mRNA of the disclosure), may play a role of encapsulating the nucleic acid payloads within the LNP with high efficiency.

According to the type of the amine and the tail group comprised in the ionizable lipid, (i) the nucleic acid encapsulation efficiency, (ii) PDI (polydispersity index). and/or (iii) the nucleic acid delivery efficiency to tissue and/or cells constituting an organ (for example, hepatocytes or liver sinusoidal endothelial cells in the liver) of the LNP may be different. In certain embodiments, the ionizable lipid is an ionizable cationic lipid, and comprises from about 25 mol % to about 66 mol % of the total lipid present in the particle.

The LNP comprising an ionizable lipid comprising an amine may have one or more kinds of the following characteristics. (1) the ability to encapsulate a nucleic acid with high efficiency; (2) uniform size of prepared particles (or having a low PDI value); and/or (3) excellent nucleic acid delivery efficiency to organs such as liver, lung, heart, spleen, bone marrow, as well as to tumors, and/or cells constituting such organs (for example, hepatocytes, LSEC, cardiac cells, cancer cells, etc.).

In particular embodiments, the cationic lipid form plays a crucial role both in nucleic acid encapsulation through electrostatic interactions and intracellular release by disrupting endosomal membranes. The nucleic acid payloads are encapsulated within the LNP by the ionic interactions they form with the positively charged cationic lipid. Non-limiting examples of ionizable cationic lipid components utilized in the LNP of the disclosure are selected from DLin-MC3-DMA (heptatriaconta-6,9,28,31-tetraen-19-yl4-(dimethylamino)butanoate), DLin-KC2-DMA (2,2-dilinoleyl-4-(2-dimethylaminoethyl)-[1,3]-dioxolane), and TNT (1,3,5-triazinane-2,4,6-trione) and TT (N1,N3,N5-tris(2-aminoethyl)benzene-1,3,5-tricarboxamide). Non-limiting examples of helper lipids utilized in the LNP of the disclosure are selected from DSPC (1,2-distearoyl-sn-glycero-3-phosphocholine), POPC (2-Oleoyl-1-palmitoyl-sn-glycero-3-phosphocholine) and DOPE (1,2-Dioleoyl-sn-glycero-3-phosphoethanolamine), 1,2-dioleoyl-sn-glycero-3-phospho-(1′-rac-glycerol) DOPG, 1,2-Dimyristoyl-sn-glycero-3-phosphoethanolamine (DMPE), 1,2-dilauroyl-sn-glycero-3-phosphocholine (DLPC), sphingolipid, and ceramide. Cholesterol and PEG-DMG ((R)-2,3-bis(octadecyloxy)propyl-1-(methoxy polyethylene glycol 2000) carbamate), PEG-DSG (1,2-Distearoyl-rac-glycero-3-methylpolyoxyethylene glycol 2000), or DSPE-PEG2k (1,2-distearoyl-sn-glycero-3-phosphoethanolamine-N-[amino(polyethylene glycol)-2000]), are components utilized in the LNP of the disclosure for the stability, circulation, and size of the LNP.

In some embodiments, the cationic lipid in the LNP of the disclosure comprises a tertiary amine. In some embodiments, the tertiary amine includes alkyl chains connected to N of the tertiary amine with ether linkages. In some embodiments, the alkyl chains comprise C12-C30 alkyl chains having 0 to 3 double bonds. In some embodiments, the alkyl chains comprise C16-C22 alkyl chains. In some embodiments, the alkyl chains comprise C18 alkyl chains. A number of cationic lipids and related analogs have been described in U.S. Patent Publication Nos. 20060083780, 20060240554, 20110117125, 20190336608, 20190381180 and 20200121809; U.S. Pat. Nos. 5,208,036; 5,264,618; 5,279,833; 5,283,185; 5,753,613; 5,785,992; 9,738,593; 10,106,490; 10,166,298; 10,221,127; and 11,219,634; and PCT Publication No. WO 96/10390, the disclosures of which are herein incorporated by reference in their entirety.

In some embodiments, the cationic lipid in the LNP of the disclosure may comprise, for example, one or more ionizable cationic lipids wherein the ionizable cationic lipid is a dialkyl lipid. In other embodiments, the ionizable cationic lipid is a tetraalkyl lipid.

In some embodiments, the cationic lipid in the LNP of the disclosure is selected from 1,2-dilinoleyloxy-N,N-dimethylaminopropane (DLinDMA), 1,2-dilinolenyloxy-N,N-dimethylaminopropane (DLenDMA), 2,2-dilinoleyl-4-(2-dimethylaminoethyl)-[1,3]-dioxolane (DLin-K-C2-DMA), 2,2-dilinoleyl-4-(3-dimethylaminopropyl)-[1,3]-dioxolane (DLin-K-C3-DMA), 2,2-dilinoleyl-4-(4-dimethylaminobutyl)-[1,3]-dioxolane (DLin-K-C4-DMA), 2,2-dilinoleyl-5-dimethylaminomethyl-[1,3]-dioxane (DLin-K6-DMA), 2,2-dilinoleyl-4-N-methylpepiazino-[1,3]-dioxolane (DLin-K-MPZ), 2,2-dilinoleyl-4-dimethylaminomethyl-[1,3]-dioxolane (DLin-K-DMA), 1,2-dilinoleylcarbamoyloxy-3-dimethylaminopropane (DLin-C-DAP), 1,2-dilinoleyoxy-3-(dimethylamino)acetoxypropane (DLin-DAC), 1,2-dilinoleyoxy-3-morpholinopropane (DLin-MA), 1,2-dilinoleoyl-3-dimethylaminopropane (DLinDAP), 1,2-dilinoleylthio-3-dimethylaminopropane (DLin-S-DMA), 1-linoleoyl-2-linoleyloxy-3-dimethylaminopropane (DLin-2-DMAP), 1,2-dilinoleyloxy-3-trimethylaminopropane chloride salt (DLin-TMA.Cl), 1,2-dilinoleoyl-3-trimethylaminopropane chloride salt (DLin-TAP.Cl), 1,2-dilinoleyloxy-3-(N-methylpiperazino)propane (DLin-MPZ), 3-(N,N-dilinoleylamino)-1,2-propanediol (DLinAP), 3-(N,N-dioleylamino)-1,2-propanedio (DOAP), 1,2-dilinoleyloxo-3-(2-N,N-dimethylamino)ethoxypropane (DLin-EG-DMA), N,N-dioleyl-N,N-dimethylammonium chloride (DODAC), 1,2-dioleyloxy-N,N-dimethylaminopropane (DODMA), 1,2-distearyloxy-N,N-dimethylaminopropane (DSDMA), N-(1-(2,3-dioleyloxy)propyl)-N,N,N-trimethylammonium chloride (DOTMA), N,N-distearyl-N,N-dimethylammonium bromide (DDAB), N-(1-(2,3-dioleoyloxy)propyl)-N,N,N-trimethylammonium chloride (DOTAP), 3-(N—(N′,N′-dimethylaminoethane)-carbamoyl)cholesterol (DC-Chol), N-(1,2-dimyristyloxyprop-3-yl)-N,N-dimethyl-N-hydroxyethyl ammonium bromide (DMRIE), 2,3-dioleyloxy-N-[2(spermine-carboxamido)ethyl]-N,N-dimethyl-1-propanaminiumtrifluoroacetate (DOSPA), dioctadecylamidoglycyl spermine (DOGS), 3-dimethylamino-2-(cholest-5-en-3-beta-oxybutan-4-oxy)-1-(cis,cis-9,12-octadecadienoxy)propane (CLinDMA), 2-[5′-(cholest-5-en-3-beta-oxy)-3′-oxapentoxy)-3-dimethyl-1-(cis,cis-9′,1-2′-octadecadienoxy)propane (CpLinDMA), N,N-dimethyl-3,4-dioleyloxybenzylamine (DMOBA), 1,2-N,N′-dioleylcarbamyl-3-dimethylaminopropane (DOcarbDAP), 1,2-N,N′-dilinoleylcarbamyl-3-dimethylaminopropane (DLincarbDAP), and any combination of the forgoing.

In some embodiments, the cationic lipid in the LNP of the disclosure is selected from heptatriaconta-6,9,28,31-tetraen-19-yl4-(dimethylamino)butanoate (DLin-MC3-DMA), 2,2-dilinoleyl-4-(2-dimethylaminoethyl)-[1,3]-dioxolane (DLin-KC2-DMA), (1,3,5-triazinane-2,4,6-trione) (TNT), N1,N3,N5-tris(2-aminoethyl)benzene-1,3,5-tricarboxamide (TT), and any combination of the forgoing.

In some embodiments, the N/P ratio (nitrogen from the cationic/ionizable lipid and phosphate from the nucleic acid) in the LNP of the disclosure is in the range of is about 3:1 to 7:1, or about 4:1 to 6:1, or is 3:1, or is 4:1, or is 5:1, or is 6:1, or is 7:1, or is 8:1, or is 9:1.

### Conjugated Lipid

In some embodiments, the LNPs and LNP compositions of the present disclosure include at least one conjugated lipid. In some embodiments, the conjugated lipid may be selected from a polyethyleneglycol (PEG)-lipid conjugate, a polyamide (ATTA)-lipid conjugate, a cationic-polymer-lipid conjugate (CPL), and any combination of the foregoing. In some cases, conjugated lipids can inhibit aggregation of the LNPs of the disclosure.

In some embodiments, the conjugated lipid of the LNP of the disclosure comprises a pegylated lipid. The terms “polyethyleneglycol (PEG)-lipid conjugate,” “pegylated lipid” “lipid-PEG conjugate”, “lipid-PEG”, “PEG-lipid”, “PEG-lipid”, or “lipid-PEG” are used interchangeably herein and refer to a lipid attached to a polyethylene glycol (PEG) polymer which is a hydrophilic polymer. The pegylated lipid contributes to the stability of the LNPs and LNP compositions and reduces aggregation of the LNPs. In other embodiments, the lipid of the LNP comprises peptide modified PEG lipids that are used for targeting cell surface receptors Ex: DSPE-PEG-RGD, DSPE-PEG-Transferrin, DSPE-PEG-cholesterol.

As the PEG-lipid can form the surface lipid, the size of the LNP can be readily varied by varying the proportion of surface (PEG) lipid to the core (ionizable cationic) lipids. In some embodiments, the PEG-lipid of the LNP of the disclosure can be varied from −1 to 5 mol % to modify particle properties such as size, stability, and circulation time.

The lipid-PEG conjugate contributes to the particle stability in serum of the nanoparticle within the LNP, and plays a role of preventing aggregation between nanoparticles. In addition, the lipid-PEG conjugate may protect nucleic acids, such as mRNAs encoding the engineered CasX proteins of the disclosure, or ERSs of the disclosure, from degrading enzymes during in vivo delivery of the nucleic acids and enhance the stability of the nucleic acids in vivo and increase the half-life of the delivered nucleic acids encapsulated in the nanoparticle. Examples of PEG-lipid conjugates include, but are not limited to, PEG-DAG conjugates, PEG-DAA conjugates, and mixtures thereof. In certain embodiments, the PEG-lipid conjugate is selected from the group consisting of a PEG-diacylglycerol (PEG-DAG) conjugate, a PEG-dialkyloxypropyl (PEG-DAA) conjugate, a PEG-phospholipid conjugate, a PEG-ceramide (PEG-Cer) conjugate, and a mixture thereof.

In some embodiments, the pegylated lipid of the LNP of the disclosure is selected from a PEG-ceramide, a PEG-diacylglycerol, a PEG-dialkyloxypropyl, a PEG-dialkoxypropylcarbamate, a PEG-phosphatidylethanoloamine, a PEG-phospholipid, a PEG-succinate diacylglycerol, and any combination of the foregoing.

In some embodiments, the pegylated lipid of the LNP of the disclosure is a PEG-dialkyloxypropyl. In some embodiments, the pegylated lipid is selected from PEG-didecyloxypropyl (C10), PEG-dilauryloxypropyl (C12), PEG-dimyristyloxypropyl (C14), PEG-dipalmityloxypropyl (C16), PEG-distearyloxypropyl (C18), and any combination of the foregoing.

In other embodiments, the lipid-PEG conjugate of the LNP of the disclosure may be PEG bound to phospholipid such as phosphatidylethanolamine (PEG-PE), PEG conjugated to ceramide (PEG-CER, ceramide-PEG conjugate, ceramide-PEG, cholesterol or PEG conjugated to derivative thereof, PEG-c-DOMG, PEG-DMG, PEG-DLPE, PEG-DMPE, PEG-DPPC, PEG-DSPE(DSPE-PEG), and a mixture thereof, and for example, may be C16-PEG2000 ceramide (N-palmitoyl-sphingosine-1-{succinyl[methoxy(polyethylene glycol)2000]}), DMG-PEG 2000, 14:0 PEG2000 PE.

In some embodiments, the pegylated lipid of the LNP of the disclosure is selected from 1-(monomethoxy-polyethyleneglycol)-2,3-dimyristoylglycerol, 4-O-(2′,3′-di(tetradecanoyloxy)propyl-1-O-(ω-methoxy(polyethoxy)ethyl)butanedioate (PEG-S-DMG), ω-methoxy(polyethoxy)ethyl-N-(2,3-di(tetradecanoxy)propyl)carbamate, 2,3-di(tetradecanoxy)propyl-N-(ω-methoxy(polyethoxy)ethyl)carbamate, and any combination of the foregoing.

In some embodiments, the pegylated lipid of the LNP of the disclosure is selected from mPEG2000-1,2-di-O-alkyl-sn3-carbomoylglyceride (PEG-C-DOMG), 1-[8′-(1,2-dimyristoyl-3-propanoxy)-carboxamido-3′,6′-dioxaoctanyl]carbamoyl-w-methyl-poly(ethylene glycol) (2 KPEG-DMG), and any combination of the foregoing.

In some embodiments, the PEG is directly attached to the lipid of the pegylated lipid. In other embodiments, the PEG is attached to the lipid of the pegylated lipid by a linker moiety selected from an ester-free linker moiety or an ester-containing linker moiety. Non-limiting examples of the ester-free linker moiety include amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, disulfide and combinations thereof. For example, the linker may contain a carbamate linker moiety and an amido linker moiety. Non-limiting examples of the ester-containing linker moiety include carbonate (—OC(O)O—), succinoyl, phosphate ester (—O—(O)POH—O—), sulfonate ester, and combinations thereof.

The PEG moiety of the pegylated lipid of the LNP of the disclosure described herein may have an average molecular weight ranging from about 550 daltons to about 10,000 daltons. In certain embodiments, the PEG moiety has an average molecular weight of from about 750 daltons to about 5,000 daltons, about 1,000 daltons to about 4,000 daltons, about 1,500 daltons to about 3,000 daltons, about 750 daltons to about 3,000 daltons, or about 1750 daltons to about 2,000 daltons.

In some embodiments, the conjugated lipid (e.g., pegylated lipid) comprises from about 1 mol % to about 65 mol %, from about 2 mol % to about 50 mol %, from about 5 mol % to about 40 mol %, or from about 5 mol % to about 20 mol % of the total lipid present in the LNPs and/or LNP compositions. In certain embodiments, the conjugated lipid comprises from about 0.5 mol % to about 3 mol % of the total lipid present in the particle.

In additional embodiments, the conjugated lipid (e.g., pegylated lipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

For the lipid in the lipid-PEG conjugate of the LNP of the disclosure, any lipid capable of binding to polyethyleneglycol may be used without limitation, and the phospholipid and/or cholesterol which are other elements of the LNP may be also used. In some embodiments, the lipid in the lipid-PEG conjugate may be ceramide, dimyristoylglycerol (DMG), succinoyl-diacylglycerol (s-DAG), distearoylphosphatidylcholine (DSPC), distearoylphosphatidylethanolamine (DSPE), or cholesterol, but not limited thereto.

In the lipid-PEG conjugate of the LNP of the disclosure, the PEG may be directly conjugated to the lipid or linked to the lipid via a linker moiety. Any linker moiety suitable for binding PEG to the lipid may be used, and for example, includes an ester-free linker moiety and an ester-containing linker moiety. The ester-free linker moiety includes not only amido (—C(O)NH—), amino (—NR—), carbonyl (—C(O)—), carbamate (—NHC(O)O—), urea (—NHC(O)NH—), disulfide (—S—S—), ether (—O—), succinyl (—(O)CCH2CH2C(O)—), succinamidyl (—NHC(O)CH2CH2C(O)NH—), ether, disulfide but also combinations thereof (for example, a linker containing both a carbamate linker moiety and an amido linker moiety), but not limited thereto. The ester-containing linker moiety includes for example, carbonate (—OC(O)O—), succinoyl, phosphate ester (—O—(O)POH—O—), sulfonate ester, and combinations thereof, but not limited thereto.

### Steroids

In some embodiments, the LNPs and LNP compositions of the present disclosure include at least one steroid or derivative thereof. In some embodiments, the steroid comprises cholesterol. In some embodiments, the LNPs and LNP compositions comprise a cholesterol derivative selected from cholestanol, cholestanone, cholestenone, coprostanol, cholesteryl-2′-hydroxyethyl ether, cholesteryl-4′-hydroxybutyl ether, and any combination of the foregoing. 7 In some embodiments, the steroid (e.g., cholesterol) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the steroid (e.g., cholesterol) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions

### Additional Lipid/Helper Lipid or Structural Lipid

In some embodiments, the LNPs and LNP compositions of the present disclosure include at least one helper lipid. In some embodiments, the helper lipid is non-cationic lipid selected from an anionic lipid, a neutral lipid, or both. In some embodiments, the helper lipid comprises at least one phospholipid. In some embodiments, the phospholipid is selected from an anionic phospholipid, a neutral phospholipid, or both. The phospholipid of the elements of the LNPs and LNP compositions can play a role in covering and protecting a core of the LNP formed by interaction of the cationic lipid and nucleic acid in the LNP, and may facilitate cell membrane permeation and endosomal escape during intracellular delivery of the nucleic acid by binding to the phospholipid bilayer of a target cell. A phospholipid which can promote fusion of the LNP to a cell may include without limitation, any of the phospholipids selected from the group described below.

In some embodiments, the LNP include helper lipids that are used for targeting cell surface receptors Ex: DSPE-RGD, DSPE-cRGD, DSPE-Chol. Also molecules such as 18:0 Lyso-PC and 18:2 Lyso-PC.

In some embodiments, the LNPs and LNP compositions comprise at least one phospholipid selected from, but not limited to, dipalmitoyl-phosphatidylcholine (DPPC), distearoyl-phosphatidylcholine (DSPC), dioleoyl-phosphatidylethanolamine (DOPE), dioleoyl-phosphatidylcholine (DOPC), dioleoyl-phosphatidylglycerol (DOPG), palmitoyloleoyl-phosphatidylcholine (POPC), palmitoyloleoyl-phosphatidylethanolamine (POPE), palmitoyloleyol-phosphatidylglycerol (POPG), dipalmitoyl-phosphatidylethanolamine (DPPE), dipalmitoyl-phosphatidylglycerol (DPPG), dimyristoyl-phosphatidylethanolamine (DMPE), distearoyl-phosphatidylethanolamine (DSPE), monomethyl-phosphatidylethanolamine, dimethyl-phosphatidylethanolamine, dielaidoyl-phosphatidylethanolamine (DEPE), stearoyloleoyl-phosphatidylethanolamine (SOPE), egg phosphatidylcholine (EPC), phosphatidylethanolamine (PE), 1,2-dioleoyl-sn-glycero-3-phosphoethanolamine, 1-palmitoyl-2-oleoyl-sn-glycero-3-phosphocholine (POPC), 1,2-dioleoyl-sn-glycero-3-[phospho-L-serine](DOPS), 1,2-dioleoyl-sn-glycero-3-[phospho-L-serine], and any combination of the foregoing. In one example, the LNP comprising DSPC may be effective in mRNA delivery (excellent drug delivery efficacy).

In some embodiments, the helper lipid (e.g., phospholipid) of the LNP of the disclosure comprises from about 1 mol % to about 60 mol %, from about 2 mol % to about 50 mol %, from about 5 mol % to about 40 mol %, or from about 5 mol % to about 20 mol % of the total lipid present in the LNPs and/or LNP compositions. In other embodiments, the helper lipid (e.g., phospholipid) of the LNP of the disclosure comprises at least about 1, 2, 5, 10, 15, 20, 25, 30, 35, 40, 45, 50, 55, or 60 mol %, or an intermediate range of any of the foregoing, of the total lipid present in the LNPs and/or LNP compositions.

It will be appreciated that the total lipid present in the LNPs and/or LNP compositions comprises the lipids as individual or in combination of the cationic lipid or ionizable cationic lipid, the conjugated lipid, (e.g., pegylated lipid), the peptide conjugated PEG lipid, the steroid (e.g., cholesterol), peptide conjugated-structural lipid (Ex: DSPE-cRGD) and the structural lipid (e.g., phospholipid), leading from LNP formulation containing one to multi-component but not limited to one, two, three, four or five components in an LNP formulation.

The LNPs and/or LNP compositions may be prepared by dissolving the total lipids (or a portion thereof) in an organic solvent (e.g., ethanol) followed by mixing through a micromixer with the payload (e.g., nucleic acids of the systems) dissolved in an acidic buffer (e.g., pH between 1.0-6.5). At this pH the ionizable cationic lipid is positively charged and interacts with the negatively-charged nucleic acid polymers. The resulting nanostructures containing the nucleic acids are then converted to neutral LNPs when dialyzed against a neutral buffer which also includes removal of the organic solvent (e.g., ethanol) during the exchange of LNPs into physiologically relevant buffer. The LNPs and/or LNP compositions thus formed have a distinct electron-dense nanostructured core where the cationic lipids are organized into inverted micelles around the encapsulated payload, as opposed to traditional bilayer liposomal structures. In another embodiment, the LNP may form a bleb-like structure with nucleic acids in aqueous pockets along the non-electron dense lipid core.

c. Lipid Nanoparticle Properties

In some embodiments, the LNPs and/or LNP compositions comprise from about 21 mol % to about 85 mol % of the cationic lipid or ionizable cationic lipid, from about 8-65% of helper lipid, about 5-79% cholesterol and about 0.5-10% PEG lipid. In some embodiments, the LNPs and/or LNP compositions comprise from about 50 mol % to about 85 mol % of the cationic lipid or ionizable cationic lipid, from about 0.5 mol % to about 5 mol % of the conjugated lipid, (e.g., pegylated lipid), from about 0.5 mol % to about 5 mol % of the steroid (e.g., cholesterol) and from about 5 mol % to about 20 mol % of the helper lipid (e.g., phospholipid).

In some embodiments, the LNPs and/or LNP compositions of the disclosure comprise cationic lipid:helper lipid (e.g., phospholipid):steroid (e.g., cholesterol):conjugated lipid, (e.g., pegylated lipid) at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5.

In some embodiments, the LNPs and/or LNP compositions of the disclosure have a total lipid:payload ratio (mass/mass) of from about 1 to about 100. In some embodiments, the total lipid:payload ratio is about 1 to about 50, from about 2 to about 25, from about 3 to about 20, from about 4 to about 15, or from about 5 to about 10. In some embodiments, the total lipid: payload ratio is about 5 to about 15, e.g., about 5, 6, 7, 8, 9, 10, 11, 12, 13, 14, 15, or an intermediate range of any of the foregoing.

In certain embodiments, the LNPs of the disclosure comprise a total lipid: nucleic acid mass ratio of from about 5:1 to about 15:1. In some embodiments, the weight ratio of the cationic lipid and nucleic acid comprised in the LNP may be 1 to 20:1, 1 to 15:1, 1 to 10:1, 5 to 20:1, 5 to 15:1, 5 to 10:1, 7.5 to 20:1, 7.5 to 15:1, or 7.5 to 10:1.

In some embodiments, the LNP of the disclosure may comprise the cationic lipid of 20 to 50 parts by weight, the phospholipid of 10 to 30 parts by weight, cholesterol of 20 to 60 parts by weight (or 20 to 60 parts by weight), and lipid-PEG conjugate of 0.1 to 10 parts by weight (or 0.25 to 10 parts by weight, 0.5 to 5 parts by weight). Alternatively, the LNP may comprise the cationic lipid of 20 to 50% by weight, phospholipid of 10 to 60% by weight, cholesterol of 20 to 60% by weight (or 30 to 60% by weight), and lipid-PEG conjugate of 0.1 to 10% by weight (or 0.25 to 10% by weight, 0.5 to 5% by weight) based on the total nanoparticle weight. As a further alternative, the LNP may comprise the cationic lipid of 25 to 50% by weight, phospholipid of 10 to 20% by weight, cholesterol of 35 to 55% by weight, and lipid-PEG conjugate of 0.1 to 10% by weight (or 0.25 to 10% by weight, 0.5 to 5% by weight), based on the total nanoparticle weight.

In some embodiments, the LNPs of the present disclosure have a mean diameter of from about 20 to 200 nm, 20 to 180 nm, 20 to 170 nm, 20 to 150 nm, 20 to 120 nm, 20 to 100 nm, 20 to 90 nm, 30 to 200 nm, 30 to 180 nm, 30 to 170 nm, 30 to 150 nm, 30 to 120 nm, 30 to 100 nm, 30 to 90 nm, 40 to 200 nm, 40 to 180 nm, 40 to 170 nm, 40 to 150 nm, 40 to 120 nm, 40 to 100 nm, 40 to 90 nm, 40 to 80 nm, 40 to 70 nm, 50 to 200 nm, 50 to 180 nm, 50 to 170 nm, 50 to 150 nm, 50 to 120 nm, 50 to 100 nm, 50 to 90 nm, 60 to 200 nm, 60 to 180 nm, 60 to 170 nm, 60 to 150 nm, 60 to 120 nm, 60 to 100 nm, 60 to 90 nm, 70 to 200 nm, 70 to 180 nm, 70 to 170 nm, 70 to 150 nm, 70 to 120 nm, 70 to 100 nm, 70 to 90 nm, 80 to 200 nm, 80 to 180 nm, 80 to 170 nm, 80 to 150 nm, 80 to 120 nm, 80 to 100 nm, 80 to 90 nm, 90 to 200 nm, 90 to 180 nm, 90 to 170 nm, 90 to 150 nm, 90 to 120 nm, or 90 to 100 nm, or an intermediate range of any of the foregoing.

In some embodiments, the LNPs and/or LNP compositions of the disclosure have a positive charge at acidic pH and may encapsulate the payload (e.g., therapeutic agent) through electrostatic interaction produced by negative charges of the payload (e.g., therapeutic agent). The term “encapsulation,” refers to the mixture of lipids surrounding and embedding the payload (e.g., therapeutic agent) at physiological conditions, forming the LNPs. The term “encapsulation efficiency,” as used herein is the percent amount of payload (e.g., therapeutic agent) encapsulated by the LNPs. It is a measure of payload (e.g., therapeutic agent) in bulk before disruption of LNPs divided by the total amount of payload (e.g., therapeutic agent) measured in bulk post-disruption of LNPs using a surfactant based reagent such as 1-2% Triton X-100. The encapsulation efficiency of the LNPs and/or LNP compositions may be 70% or more, 75% or more, 80% or more, 85% or more, 90% or more, 91% or more, 92% or more, 94% or more, or 95% or more. In other embodiments, the encapsulation efficiency of the LNPs and/or LNP compositions is about 80% to 99%, about 85% to 98%, about 88% to 95%, about 90% to 95%, or the payload (e.g., nucleic acids of the systems) may be fully encapsulated within the lipid portion of the LNPs compositions, and thereby protected from enzymatic degradation. In some embodiments, the payload (e.g., therapeutic agent) is not substantially degraded after exposure of the LNPs and/or LNP compositions to a nuclease at 37° C. for at least about 20, 30, 45, or 60 minutes or at least about 2, 3, 4, 5, 6, 7, 8, 9, 10, 12, 14, 16, 18, 20, 22, 24, 26, 28, 30, 32, 34, or 36 hours. In some embodiments, the payload (e.g., nucleic acids of the systems) is complexed with the lipid portion of the LNPs and/or LNP compositions. The LNPs and/or LNP compositions of the present disclosure are non-toxic to mammals such as humans.

The term “fully encapsulated” indicates that the payload (e.g., the nucleic acids of the system) in the LNPs and/or LNP compositions is not significantly degraded after exposure to conditions that significantly degrade free DNA, RNA, or protein. In a fully encapsulated system, less than about 25%, more preferably less than about 10%, and most preferably less than about 5% of the payload (e.g., nucleic acids of the system) in the LNPs and/or LNP compositions is degraded by conditions that would degrade 100% of a non-encapsulated payload. “Fully encapsulated” also indicates that the LNPs and/or LNP compositions are serum-stable, and do not decompose into their component parts immediately upon exposure to serum proteins post in vivo administration and protects the cargo until endosomal escape and release into cytoplasm of the cell.

In some embodiments, the amount of the LNPs and/or LNP compositions having the payload (e.g., therapeutic agent), encapsulated therein is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the amount of the payload (e.g., the nucleic acids), encapsulated within the LNPs and/or LNP compositions is from about 30% to about 100%, from about 40% to about 100%, from about 50% to about 100%, from about 60% to about 100%, from about 70% to about 100%, from about 80% to about 100%, from about 90% to about 100%, from about 30% to about 95%, from about 40% to about 95%, from about 50% to about 95%, from about 60% to about 95%, %, from about 70% to about 95%, from about 80% to about 95%, from about 85% to about 95%, from about 90% to about 95%, from about 30% to about 90%, from about 40% to about 90%, from about 50% to about 90%, from about 60% to about 90%, from about 70% to about 90%, from about 80% to about 90%, or at least about 30%, 35%, 40%, 45%, 50%, 55%, 60%, 65%, 70%, 75%, 80%, 85%, 90%, 91%, 92%, 93%, 94%, 95%, 96%, 97%, 98%, 99%, or an intermediate range of any of the foregoing.

In some embodiments, the nucleic acids of the disclosure, such as the mRNA encoding the engineered CasX fusion protein, and/or the ERS, may be provided in a solution to be mixed with a lipid solution such that the nucleic acids may be encapsulated in the lipid nanoparticles. A suitable nucleic acid solution may be any aqueous solution containing the nucleic acid to be encapsulated at various concentrations. For example, a suitable nucleic acid solution may contain the nucleic acid (or nucleic acids) at a concentration of or greater than about 0.01 mg/ml, 0.05 mg/ml, 0.06 mg/ml, 0.07 mg/ml, 0.08 mg/ml, 0.09 mg/ml, 0.1 mg/ml, 0.15 mg/ml, 0.2 mg/ml, 0.3 mg/ml, 0.4 mg/ml, 0.5 mg/ml, 0.6 mg/ml, 0.7 mg/ml, 0.8 mg/ml, 0.9 mg/ml, 1.0 mg/ml, 1.25 mg/ml, 1.5 mg/ml, 1.75 mg/ml, or 2.0 mg/ml. In some embodiments, the nucleic acid comprises an mRNA encoding an engineered CasX, and a suitable mRNA solution may contain the mRNA at a concentration ranging from about 0.01-2.0 mg/ml, 0.01-1.5 mg/ml, 0.01-1.25 mg/ml, 0.01-1.0 mg/ml, 0.01-0.9 mg/ml, 0.01-0.8 mg/ml, 0.01-0.7 mg/ml, 0.01-0.6 mg/ml, 0.01-0.5 mg/ml, 0.01-0.4 mg/ml, 0.01-0.3 mg/ml, 0.01-0.2 mg/ml, 0.01-0.1 mg/ml, 0.05-1.0 mg/ml, 0.05-0.9 mg/ml, 0.05-0.8 mg/ml, 0.05-0.7 mg/ml, 0.05-0.6 mg/ml, 0.05-0.5 mg/ml, 0.05-0.4 mg/ml, 0.05-0.3 mg/ml, 0.05-0.2 mg/ml, 0.05-0.1 mg/ml, 0.1-1.0 mg/ml, 0.2-0.9 mg/ml, 0.3-0.8 mg/ml, 0.4-0.7 mg/ml, or 0.5-0.6 mg/ml. In some embodiments, a suitable mRNA solution may contain an mRNA at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml. In some embodiments, a suitable ERS solution may contain an ERS at a concentration up to about 5.0 mg/ml, 4.0 mg/ml, 3.0 mg/ml, 2.0 mg/ml, 1.0 mg/ml, 0.9 mg/ml, 0.8 mg/ml, 0.7 mg/ml, 0.6 mg/ml, 0.5 mg/ml, 0.4 mg/ml, 0.3 mg/ml, 0.2 mg/ml, 0.1 mg/ml, 0.05 mg/ml, 0.04 mg/ml, 0.03 mg/ml, 0.02 mg/ml, 0.01 mg/ml, or 0.05 mg/ml.

In some embodiments, the LNP may have an average diameter of 20 nm to 200 nm, 20 to 180 nm, 20 nm to 170 nm, 20 nm to 150 nm, 20 nm to 120 nm, 20 nm to 100 nm, 20 nm to 90 nm, 30 nm to 200 nm, 30 to 180 nm, 30 nm to 170 nm, 30 nm to 150 nm, 30 nm to 120 nm, 30 nm to 100 nm, 30 nm to 90 nm, 40 nm to 200 nm, 40 to 180 nm, 40 nm to 170 nm, 40 nm to 150 nm, 40 nm to 120 nm, 40 nm to 100 nm, 40 nm to 90 nm, 40 nm to 80 nm, 40 nm to 70 nm, 50 nm to 200 nm, 50 to 180 nm, 50 nm to 170 nm, 50 nm to 150 nm, 50 nm to 120 nm, 50 nm to 100 nm, 50 nm to 90 nm, 60 nm to 200 nm, 60 to 180 nm, 60 nm to 170 nm, 60 nm to 150 nm, 60 nm to 120 nm, 60 nm to 100 nm, 60 nm to 90 nm, 70 nm to 200 nm, 70 to 180 nm, 70 nm to 170 nm, 70 nm to 150 nm, 70 nm to 120 nm, 70 nm to 100 nm, 70 nm to 90 nm, 80 nm to 200 nm, 80 to 180 nm, 80 nm to 170 nm, 80 nm to 150 nm, 80 nm to 120 nm, 80 nm to 100 nm, 80 nm to 90 nm, 90 nm to 200 nm, 90 to 180 nm, 90 nm to 170 nm, 90 nm to 150 nm, 90 nm to 120 nm, or 90 nm to 100 nm for easy introduction into liver tissue, hepatocytes and/or LSEC (liver sinusoidal endothelial cells). The LNP may be sized for easy introduction into organs or tissues, including but not limited to liver, lung, heart, spleen, as well as to tumors. When the size of the LNP is smaller than the above range, it can be difficult to maintain stability as the surface area of the LNP is excessively increased, and thus delivery to the target tissue and/or drug effect may be reduced. The LNP may specifically target liver tissue. Without wishing to be bound by theory, it is thought that one mechanism by which LNP may be used to deliver therapeutic agents is through the imitation of the metabolic behaviors of natural lipoproteins, and so LNP may be usefully delivered to a subject through the lipid metabolism processes carried out by the liver. During the delivery of therapeutic agents to hepatocytes or and/or LSEC (liver sinusoidal endothelial cells), the diameter of the fenestrae leading from the sinusoidal lumen to the hepatocytes and LSEC is about 140 nm in mammals and about 100 nm in humans, so the LNP composition for therapeutic agent delivery having LNPs with a diameter in the above ranges may have excellent delivery efficiency to hepatocytes and LSEC when compared to LNP having the diameter outside the above range.

According to one example, the LNPs of the LNP composition may comprise the cationic lipid:phospholipid:cholesterol:lipid-PEG conjugate in the range described above or at a molar ratio of 20 to 50:10 to 30:30 to 60:0.5 to 5, at a molar ratio of 25 to 45:10 to 25:40 to 50:0.5 to 3, at a molar ratio of 25 to 45:10 to 20:40 to 55:0.5 to 3, or at a molar ratio of 25 to 45:10 to 20:40 to 55:1.0 to 1.5. The LNP comprising components at a molar ratio in the above range may have excellent delivery efficiency of therapeutic agents specific to cells of target organs.

In certain aspects, the LNP exhibit a positive charge under the acidic pH condition by showing a pKa of 5 to 8, 5.5 to 7.5, 6 to 7, or 6.5 to 7, and may encapsulate a nucleic acid with high efficiency by easily forming a complex with a nucleic acid through electrostatic interaction with a therapeutic agent such as a nucleic acid showing a negative charge. In such cases, the LNP may be usefully used as a composition for intracellular or in vivo delivery of a therapeutic agent (for example, nucleic acid).

Herein, “encapsulate” or “encapsulation” refers to incorporation of a therapeutic agent efficiently inside a lipid envelope, i.e., by surrounding it by the particle surface and/or embedding it within the particle interior made of various lipids that self-assemble when the polarity of the solvent surrounding them is increased. The encapsulation efficiency means the content of the therapeutic agent encapsulated in the LNP relative the total therapeutic agent content measured per given volume of the LNP formulation measured post-disruption of the LNPs.

The encapsulation of the nucleic acids of the composition in the LNP may be 70% or more, 75% or more, 80% or more, 85% or more, 90% or more, 91% or more, 92% or more, 94% or more, or 95% or more of LNP in the composition encapsulate nucleic acids. In some embodiments, the encapsulation of the nucleic acids of the composition in the LNP is such that between 80% to 99%, between 80% to 97%, between 80% to 95%, between 85% to 95%, between 87% to 95%, between 90% to 95%, between 91% or more to 95% or less, 91% or more to 94% or less, over 91% to 95% or less, 92% to 99%, between 92% to 97%, or between 92% to 95% of the LNP in the composition encapsulate nucleic acids. In some embodiments, the mRNA encoding the engineered CasX and a ERS of any of the embodiments of the disclosure are fully encapsulated in the LNP.

The target organs to which a nucleic acid is delivered by the LNP include, but are not limited to the liver, lung, heart, spleen, as well as to tumors. The LNP according to one example is liver tissue-specific and has excellent biocompatibility and can deliver the nucleic acids of a composition with high efficiency, and thus it can be usefully used in related technical fields such as lipid nanoparticle-mediated gene therapy. In a particular embodiment, the target cell to which the nucleic acids are delivered by the LNP according to one example may be a hepatocyte and/or LSEC in vivo. In other embodiments, the disclosure provides LNP formulated for delivery of the nucleic acids of the embodiments to cells ex vivo.

The disclosure provides a pharmaceutical composition comprising a plurality of LNPs comprising nucleic acids, such as mRNA encoding an engineered CasX protein and/or a ERS described herein, and a pharmaceutically acceptable carrier.

In certain embodiments, the LNP comprising the nucleic acid(s) has an electron dense core.

The disclosure provides LNP comprising one or more nucleic acids comprising: (a) an mRNA encoding the engineered CasX, and/or a ERS described herein; (b) one or more cationic lipids or ionizable cationic lipids or salts thereof comprising from about 50 mol % to about 85 mol % of the total lipid present in the LNP; (c) one or more non-cationic lipids comprising from about 13 mol % to about 49.5 mol % of the total lipid present in the LNP; and (d) one or more conjugated lipids that inhibit aggregation of LNPs comprising from about 0.5 mol % to about 2 mol % of the total lipid present in the particle. In another embodiment, the disclosure provides LNP comprising one or more nucleic acids comprising: (a) an mRNA encoding the engineered CasX, and/or a ERS described herein; (b) one or more cationic lipids or ionizable cationic lipids or salts thereof comprising from about 22 mol % to about 85 mol % of the total lipid present in the LNP; (c) one or more non-cationic/phospholipids comprising from about 10 mol % to about 70 mol % of the total lipid present in the LNP; (d) 15 mol % to about 50 mol % sterol, and (d) 1 mol % to about 5 mol % lipid-PEG or lipid-PEG-peptide in the particle. In certain embodiments the engineered CasX mRNA and ERS may be present in the same nucleic acid-lipid particle, or they may be present in different nucleic acid-lipid particles.

The disclosure provides LNP comprising one or more nucleic acids comprising: (a) an mRNA encoding the engineered CasX described herein; (b) a cationic lipid or a salt thereof comprising from about 52 mol % to about 62 mol % of the total lipid present in the LNP; (c) a mixture of a phospholipid and cholesterol or a derivative thereof comprising from about 36 mol % to about 47 mol % of the total lipid present in the LNP; and (d) a PEG-lipid conjugate comprising from about 1 mol % to about 2 mol % of the total lipid present in the LNP. In particular embodiments, the formulation is a four-component system comprising about 1.4 mol % PEG-lipid conjugate (e.g., PEG2000-C-DMA), about 57.1 mol % cationic lipid (e.g., DLin-K-C2-DMA) or a salt thereof, about 7.1 mol % DPPC (or DSPC), and about 34.3 mol % cholesterol (or derivative thereof).

In other embodiments, the LNP comprising one or more nucleic acids comprises: (a) an mRNA encoding the engineered CasX and/or a ERS of any of the embodiments described herein; (b) a cationic lipid or a salt thereof comprising from about 46.5 mol % to about 66.5 mol % of the total lipid present in the LNP; (c) cholesterol or a derivative thereof comprising from about 31.5 mol % to about 42.5 mol % of the total lipid present in the LNP; and (d) a PEG-lipid conjugate comprising from about 1 mol % to about 2 mol % of the total lipid present in the LNP. In particular embodiments, the formulation is a three-component system which is phospholipid-free and comprises about 1.5 mol % PEG-lipid conjugate (e.g., PEG2000-C-DMA), about 61.5 mol % cationic lipid (e.g., DLin-K-C2-DMA) or a salt thereof, and about 36.9 mol % cholesterol (or derivative thereof).

Additional formulations are described in PCT Publication No. WO 09/127060 and US patent publication numbers US 2011/0071208 A1 and US 2011/0076335 A1, the disclosures of which are herein incorporated by reference in their entirety.

In other embodiments, the LNP comprising one or more nucleic acids comprises: (a) an mRNA encoding the engineered CasX and a ERS of any of the embodiments described herein; (b) one or more cationic lipid or ionizable cationic lipids or salts thereof comprising from about 2 mol % to about 50 mol % of the total lipid present in the LNP; (c) one or more non-cationic lipid or ionizable cationic lipids comprising from about 5 mol % to about 90 mol % of the total lipid present in the LNP; and (d) one or more conjugated lipids that inhibit aggregation of particles comprising from about 0.5 mol % to about 20 mol % of the total lipid present in the LNP.

In other embodiments, the LNP comprising one or more nucleic acids comprises: (a) an mRNA encoding the engineered CasX and a ERS of any of the embodiments described herein; (b) a cationic lipid or a salt thereof comprising from about 30 mol % to about 50 mol % of the total lipid present in the LNP; (c) a mixture of a phospholipid and cholesterol or a derivative thereof comprising from about 47 mol % to about 69 mol % of the total lipid present in the LNP; and (d) a PEG-lipid conjugate comprising from about 1 mol % to about 3 mol % of the total lipid present in the LNP. In particular embodiments, the formulation is a four-component system which comprises about 2 mol % PEG-lipid conjugate (e.g., PEG2000-C-DMA), about 40 mol % cationic lipid (e.g., DLin-K-C2-DMA) or a salt thereof, about 10 mol % DPPC (or DSPC), and about 48 mol % cholesterol (or derivative thereof).

In other embodiments, the LNP comprising one or more nucleic acids comprises: (a) an mRNA encoding the engineered CasX and a ERS of any of the embodiments described herein; (b) one or more cationic lipid or ionizable cationic lipids or salts thereof comprising from about 50 mol % to about 65 mol % of the total lipid present in the LNP; (c) one or more non-cationic lipid or ionizable cationic lipids comprising from about 25 mol % to about 45 mol % of the total lipid present in the LNP; and (d) one or more conjugated lipids that inhibit aggregation of particles comprising from about 5 mol % to about 10 mol % of the total lipid present in the LNP.

In other embodiments, the LNP comprising one or more nucleic acids comprises: (a) an mRNA encoding the engineered CasX and a ERS of any of the embodiments described herein; (b) a cationic lipid or a salt thereof comprising from about 50 mol % to about 60 mol % of the total lipid present in the LNP; (c) a mixture of a phospholipid and cholesterol or a derivative thereof comprising from about 35 mol % to about 45 mol % of the total lipid present in the LNP; and (d) a PEG-lipid conjugate comprising from about 5 mol % to about 10 mol % of the total lipid present in the LNP.

In certain embodiments, the non-cationic lipid mixture in the formulation comprises: (i) a phospholipid of from about 10 mol % to about 70 mol % of the total lipid present in the LNP; (ii) cholesterol or a derivative thereof of from about 15 mol % to about 50 mol % of the total lipid present in the LNP; and 1-5% lipid-PEG or lipid-PEG-peptide. In particular embodiments, the formulation is a four-component system which comprises about 7 mol % PEG-lipid conjugate (e.g., PEG750-C-DMA), about 54 mol % cationic lipid (e.g., DLin-K-C2-DMA) or a salt thereof, about 7 mol % DPPC (or DSPC), and about 32 mol % cholesterol (or derivative thereof).

In other embodiments, the LNP comprising one or more nucleic acids comprises: (a) an mRNA encoding the engineered CasX and/or a ERS of any of the embodiments described herein; (b) a cationic lipid or a salt thereof comprising from about 55 mol % to about 65 mol % of the total lipid present in the LNP; (c) cholesterol or a derivative thereof comprising from about 30 mol % to about 40 mol % of the total lipid present in the LNP; and (d) a PEG-lipid conjugate comprising from about 5 mol % to about 10 mol % of the total lipid present in the LNP. In particular embodiments, the formulation is a three-component system which is phospholipid-free and comprises about 7 mol % PEG-lipid conjugate (e.g., PEG750-C-DMA), about 58 mol % cationic lipid (e.g., DLin-K-C2-DMA) or a salt thereof, and about 35 mol % cholesterol (or derivative thereof).

In other embodiments, the LNP comprising one or more nucleic acids comprises: (a) an mRNA encoding the engineered CasX and/or a ERS of any of the embodiments described herein; (b) a cationic lipid or a salt thereof comprising from about 48 mol % to about 62 mol % of the total lipid present in the LNP; (c) a mixture of a phospholipid and cholesterol or a derivative thereof, wherein the phospholipid comprises about 7 mol % to about 17 mol % of the total lipid present in the LNP, and wherein the cholesterol or derivative thereof comprises about 25 mol % to about 40 mol % of the total lipid present in the LNP; and (d) a PEG-lipid conjugate comprising from about 0.5 mol % to about 3.0 mol % of the total lipid present in the LNP.

### X. Compositions, Kits and Articles of Manufacture

In some embodiments, the disclosure provides a composition comprising an ERS of any of the embodiments described herein and a linked targeting sequence of at least 15 to 20 nucleotides, wherein the targeting sequence is complementary to a target nucleic acid of a gene.

In some embodiments, the disclosure provides a composition comprising an engineered CasX of any of the embodiments described herein.

In some embodiments, the disclosure provides a composition comprising an RNP of an ERS and linked targeting sequence and an engineered CasX of any of the embodiments described herein.

In some embodiments, the disclosure provides pharmaceutical compositions comprising an engineered CasX protein and a ERS of any of the embodiments of the disclosure and a linked targeting sequence complementary to a target nucleic acid of a gene, together with one or more pharmaceutically suitable excipients. In some embodiments, the pharmaceutical composition is formulated for a route of administration selected from the group consisting of intravenous, intraportal vein injection, intraperitoneal, intramuscular, subcutaneous, intraocular, and oral routes. In one embodiment, the pharmaceutical composition is in a liquid form or a frozen form. In another embodiment, the pharmaceutical composition is in a pre-filled syringe for a single injection. In another embodiment, the pharmaceutical composition is in solid form, for example the pharmaceutical composition is lyophilized.

In another aspect, provided herein are kits comprising the compositions of the embodiments described herein. In some embodiments, the kit comprises an engineered CasX protein and one or a plurality of ERS of any of the embodiments of the disclosure comprising a targeting sequence complementary to a target nucleic acid of a gene, a pharmaceutically suitable excipient and a suitable container (for example a tube, vial or plate). In other embodiments, the kit comprises a nucleic acid encoding an engineered CasX protein and one or a plurality of ERS of any of the embodiments of the disclosure comprising a targeting sequence complementary to a target nucleic acid of a gene, a pharmaceutically suitable excipient and a suitable container. In other embodiments, the kit comprises a vector comprising a nucleic acid encoding an engineered CasX protein and one or a plurality of ERS of any of the embodiments of the disclosure comprising a targeting sequence complementary to a target nucleic acid of a gene, a pharmaceutically suitable excipient and a suitable container. In other embodiments, the kit comprises an mRNA encoding an engineered CasX protein and one or a plurality of ERS of any of the embodiments of the disclosure comprising a targeting sequence complementary to a target nucleic acid of a gene formulated as an LNP, a pharmaceutically suitable excipient and a suitable container. In still other embodiments, the kit comprises a XDP comprising an engineered CasX protein and one or a plurality of ERS of any of the embodiments of the disclosure comprising a targeting sequence complementary to a target nucleic acid of a gene, a pharmaceutically suitable excipient and a suitable container. In still other embodiments, the kit comprises an AAV vector comprising a sequence encoding an engineered CasX protein and one or a plurality of ERS of any of the embodiments of the disclosure comprising a targeting sequence complementary to a target nucleic acid of a gene, a pharmaceutically suitable excipient and a suitable container.

In some embodiments, the kit further comprises a buffer, a nuclease inhibitor, a protease inhibitor, a liposome, a therapeutic agent, a label, a label visualization reagent, or any combination of the foregoing. In some embodiments, the kit further comprises a pharmaceutically acceptable carrier, diluent or excipient.

In some embodiments, the kit comprises appropriate control compositions for gene modifying applications, and instructions for use.

The present description sets forth numerous exemplary configurations, methods, parameters, and the like. It should be recognized, however, that such description is not intended as a limitation on the scope of the present disclosure, but is instead provided as a description of exemplary embodiments. Embodiments of the present subject matter described above may be beneficial alone or in combination, with one or more other aspects or embodiments. Without limiting the foregoing description, certain non-limiting embodiments of the disclosure are provided below. As will be apparent to those of skill in the art upon reading this disclosure, each of the individually numbered embodiments may be used or combined with any of the preceding or following individually numbered embodiments. This is intended to provide support for all such combinations of embodiments and is not limited to combinations of embodiments explicitly provided below:

### EXEMPLARY EMBODIMENTS

Embodiment I-1. A guide RNA (gRNA) scaffold comprising a sequence having at least 60%, at least 70%, at least 80%, at least 90%, at least 95%, at least 98%, at least 99%, or 100% sequence identity to any one of the sequences selected from the group consisting of SEQ ID NOS: 29-81.

Embodiment I-2. The gRNA scaffold of embodiment I-1, comprising a sequence selected from the group consisting of SEQ ID NOS: 29-81.

Embodiment I-3. The gRNA scaffold of embodiment I-1, comprising a sequence having one or more modifications relative to SEQ ID NO: 17, wherein the one or more modifications result in an improved characteristic.

Embodiment I-4. The gRNA scaffold of embodiment I-3, wherein the one or more modifications comprise one or more nucleotide substitutions, insertions, and/or deletions as set forth in Table 8.

Embodiment I-5. The gRNA scaffold of embodiment I-3 or I-4, wherein the improved characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, optionally in an in vitro assay.

Embodiment I-6. The gRNA scaffold of any one of embodiments I-3 to I-5, wherein the gRNA scaffold exhibits an improved enrichment score (log 2) of at least about 2.0, at least about 2.5, at least about 3, or at least about 3.5 greater compared to the score of the gRNA scaffold of SEQ ID NOS: 17 or 18 in an in vitro assay.

Embodiment I-7. The gRNA scaffold of embodiment I-1, comprising a sequence having one or more modifications relative to SEQ ID NO: 18, wherein the one or more modifications result in an improved characteristic.

Embodiment I-8. The gRNA scaffold of embodiment I-7, wherein the one or more modifications comprise one or more nucleotide substitutions, insertions, and/or deletions as set forth in Table 9.

Embodiment I-9. The gRNA scaffold of embodiment I-7 or I-8, wherein the improved characteristic is one or more functional properties selected from the group consisting of increased editing activity, increased pseudoknot stem stability, increased triplex region stability, increased scaffold stem stability, extended stem stability, reduced off-target folding intermediates, and increased binding affinity to a Class 2, Type V CRISPR protein, optionally in an in vitro assay.

Embodiment I-10. The gRNA scaffold of any one of embodiments I-7 to I-9, wherein the gRNA scaffold exhibits an improved enrichment score (log 2) of at least about 1.2, at least about 1.5, at least about 2.0, at least about 2.5, at least about 3, or at least about 3.5 greater compared to the score of the gRNA scaffold of SEQ ID NOS: 17 or 18 selected from the group consisting of C9, U11, C17, U24, A29, U54, G64, A88, and A95.

Embodiment I-11. The gRNA scaffold of embodiment I-10, comprising one or more modifications relative to the sequence of SEQ ID NOS: 17 or 18 selected from the group consisting of C9U, U11C, C17G, U24C, A29C, an insertion of G at position 54, an insertion of C at position 64, A88G, and A95G.

Embodiment I-12. The gRNA scaffold of embodiment I-11, comprising modifications relative to the sequence of SEQ ID NOS: 17 or 18 consisting of C9U, U11C, C17G, U24C, A29C, an insertion of G at position 54, an insertion of C at position 64, A88G, and A95G.

Embodiment I-13. The gRNA scaffold of any one of embodiments I-3 to I-12, wherein the improved characteristic is selected from the group consisting of pseudoknot stem stability, triplex region stability, scaffold bubble stability, extended stem stability, and binding affinity to a Class 2, Type V CRISPR protein.

Embodiment I-14. The gRNA scaffold of embodiment I-13, wherein the insertion of C at position 64 and the A88G substitution relative to the sequence of SEQ ID NOS: 17 or 18 resolves an asymmetrical bulge element of the extended stem, enhancing the stability of the extended stem of the gRNA scaffold.

Embodiment I-15. The gRNA scaffold of embodiment I-13, wherein the substitutions of U11C, U24C, and A95G increase the stability of the triplex region of the gRNA scaffold.

Embodiment I-16. The gRNA scaffold of embodiment I-13, wherein the substitution of A29C increases the stability of the pseudoknot stem.

Embodiment I-17. The gRNA scaffold of embodiment I-1 or I-2, wherein the gRNA scaffold comprises one or more heterologous RNA sequences in the extended stem.

Embodiment I-18. The gRNA scaffold of embodiment I-17, wherein the heterologous RNA is selected from the group consisting of a MS2 hairpin, Qβ hairpin, U1 hairpin II, Uvsx hairpin, and a PP7 stem loop, or sequence variants thereof.

Embodiment I-19. The gRNA scaffold of embodiment I-17 or I-18, wherein the heterologous RNA sequence increases the stability of the gRNA.

Embodiment I-20. The gRNA scaffold of embodiment I-17 or I-18, wherein the heterologous RNA is capable of binding a protein, a RNA, a DNA, or a small molecule.

Embodiment I-21. The gRNA scaffold of any one of embodiments I-17 to I-20, wherein the gRNA scaffold comprises a Rev response element (RRE) or a portion thereof.

Embodiment I-22. The gRNA scaffold of embodiment I-21, wherein the RRE or portion thereof is selected from the group consisting of Stem IIB of the RRE having sequence UGGGCGCAGCGUCAAUGACGCUGACGGUACA (SEQ ID NO: 353), Stem II-V of the RRE having sequence CAGGAAGCACUAUGGGCGCAGCGUCAAUGACGCUGACGGUACAGGCCAGACAAU UAUUGUCUGGUAUAGUGCAGCAGCAGAACAAUUUGCUGAGGGCUAUUGAGGCGC AACAGCAUCUGUUGCAACUCACAGUCUGGGGCAUCAAGCAGCUCCAGGCAAGAA UCCUG (SEQ ID NO: 355), Stem II of the RRE having sequence GCACUAUGGGCGCAGCGUCAAUGACGCUGACGGUACAGGCCAGACAAUUAUUGU CUGGUAUAGUGC (SEQ ID NO: 354), Rev-binding element (RBE) of Stem IIB having sequence GCUGACGGUACAGGC (SEQ ID NO: 356), and full-length RRE having sequence AGGAGCUUUGUUCCUUGGGUUCUUGGGAGCAGCAGGAAGCACUAUGGGCGCAGC GUCAAUGACGCUGACGGUACAGGCCAGACAAUUAUUGUCUGGUAUAGUGCAGCA GCAGAACAAUUUGCUGAGGGCUAUUGAGGCGCAACAGCAUCUGUUGCAACUCAC AGUCUGGGGCAUCAAGCAGCUCCAGGCAAGAAUCCUGGCUGUGGAAAGAUACCU AAAGGAUCAACAGCUCCU (SEQ ID NO: 357).

Embodiment I-23. The gRNA of any one of embodiments I-1 to I-22, wherein the gRNA scaffold comprises one or more thymines (T).

Embodiment I-24. A gRNA comprising the gRNA scaffold of any one of embodiments I-1 to I-23, and a targeting sequence at the 3′ end of the gRNA scaffold that is complementary to a target nucleic acid sequence.

Embodiment I-25. The gRNA of embodiment I-24, wherein the targeting sequence has 10, 11, 12, 13, 14, 15, 16, 17, 18, 19, 20, 21, 22, 23, 24, 25, 26, 27, 28, 29, or 30 nucleotides.

Embodiment I-26. The gRNA of embodiment I-25, wherein the targeting sequence has 18, 19, or 20 nucleotides.

Embodiment I-27. The gRNA of any one of embodiments I-24 to I-26, wherein the gRNA is capable of forming a ribonucleoprotein (RNP) complex with a Class 2, Type V CRISPR protein.

Embodiment I-28. An engineered Class 2, Type V CRISPR protein comprising:
a. a NTSB domain comprising a sequence of QPASKKIDQNKLKPEMDEKGNLTTAGFACSQCGQPLFVYKLEQVSEKGKAYTNYFGRC NVAEHEKLILLAQLKPEKDSDEAVTYSLGKFGQ (SEQ ID NO: 145), or a sequence having at least 80% at least 90%, at least 95%, at least 96%, at least 97%, at least 98% or at least 99% identity thereto;
b. a helical I-II domain comprising a sequence of RALDFYSIHVTKESTHPVKPLAQIAGNRYASGPVGKALSDACMGTIASFLSKYQDIIIEH QKVVKGNQKRLESLRELAGKENLEYPSVTLPPQPHTKEGVDAYNEVIARVRMWVNLN LWQKLKLSRDDAKPLLRLKGFPSF (SEQ ID NO: 163), or a sequence having at least 80%, at least 90%, at least 95%, at least 96%, at least 97%, at least 98% or at least 99% identity thereto;
c. a helical II domain comprising a sequence of PLVERQANEVDWWDMVCNVKKLINEKKEDGKVFWQQNLAGYKRQEALRPYLSSEEDR KKGKKFARYQLGDLLLHLEKKHGEDWGKVYDEAWERIDKKVEGLSKHIKLEEERRSE DAQSKAALTDWLRAKASFVIEGLKEADKDEFCRCELKLQKWYGDLRGKPFAIEAE (SEQ ID NO: 161), or a sequence having at least 80%, at least 90%, at least 95%, at least 96%, at least 97%, at least 98% or at least 99% identity thereto; and
d. a RuvC-I domain comprising a sequence of SSNIKPMNLIGVDRGENIPAVIALTDPEGCPLSRFKDSLGNPTHILRIGESYKEKQRTIQAK KEVEQRRAGGYSRKYASKAKNLADDMVRNTARDLLYYAVTQDAMLIFENLSRGFGRQ GKRTFMAERQYTRMEDWLTAKLAYEGLPSKTYLSKTLAQYTSKTC (SEQ ID NO: 162), or a sequence having at least 80%, at least 90%, at least 95%, at least 96%, at least 97%, at least 98% or at least 99% identity thereto.

Embodiment I-29. The Class 2, Type V CRISPR protein of embodiment I-28, wherein the CRISPR protein comprises an OBD-I domain comprising a sequence of QEIKRINKIRRRLVKDSNTKKAGKTGPMKTLLVRVMTPDLRERLENLRKKPENIPQ (SEQ ID NO: 152), or a sequence having at least 80%, at least 90%, at least 95%, at least 96%, at least 97%, at least 98%, or at least 99% identity thereto.

Embodiment I-30. The Class 2, Type V CRISPR protein of embodiment I-28 or I-29, wherein the CRISPR protein comprises an OBD-II domain comprising a sequence of
NSILDISGFSKQYNCAFIWQKDGVKKLNLYLIINYFKGGKLRFKKIKPEAFEANRFYTVINKKSGEIV PMEVNFNFDDPNLIILPLAFGKRQGREFIWNDLLSLETGSLKLANGRVIEKTLYNRRTRQDEPALF VALTFERREVLD (SEQ ID NO: 164), or a sequence having at least 80%, at least 90%, at least 95%, at least 96%, at least 97%, at least 98%, or at least 99% identity thereto.

Embodiment I-31. The Class 2, Type V CRISPR protein of any one of embodiments I-28 to I-30, wherein the CRISPR protein comprises a helical I-I domain comprising a sequence of PISNTSRANLNKLLTDYTEMKKAILHVYWEEFQKDPVGLMSRVA (SEQ ID NO: 153), or a sequence having at least 80%, at least 90%, at least 95%, at least 96%, at least 97%, at least 98%, or at least 99% identity thereto.

Embodiment I-32. The Class 2, Type V CRISPR protein of any one of embodiments I-28 to I-31, wherein the CRISPR protein comprises a TSL domain comprising a sequence of SNCGFTITSADYDRVLEKLKKTATGWMT TINGKELKVEGQITYYNRYKRQNVVKDLSV ELDRLSEESVNNDISSWTKGRSGEALSLLKKRFSHRPVQEKFVCLNCGFETH (SEQ ID NO: 159), or a sequence having at least 80%, at least 90%, at least 95%, at least 96%, at least 97%, at least 98%, or at least 99% identity thereto.

Embodiment I-33. The Class 2, Type V CRISPR protein of any one of embodiments I-28 to I-32, wherein the CRISPR protein comprises a RuvC-II domain comprising a sequence of
ADEQAALNIARSWLFLRSQEYKKYQTNKTTGNTDKRAFVETWQSFYRKKLKEV WKPAV (SEQ ID NO: 160), or a sequence having at least 80%, at least 90%, at least 95%, at least 96%, at least 97%, at least 98%, or at least 99% identity thereto.

Embodiment I-35. The Class 2, Type V CRISPR protein of embodiment I-33, comprising the sequence of SEQ ID NOS: 95-142, or a sequence having at least 80%, at least 90%, at least 95%, at least 96%, at least 97%, at least 98%, or at least 99% identity thereto.

Embodiment I-36. The Class 2, Type V CRISPR protein of any one of embodiments I-28 to I-35, wherein the Class 2, Type V CRISPR protein comprises at least one modification in one or more domains.

Embodiment I-37. The Class 2, Type V CRISPR protein of embodiment I-36, wherein the at least one modification comprises:
a. at least one amino acid substitution in a domain;
b. at least one amino acid deletion in a domain;
c. at least one amino acid insertion in a domain; or
d. any combination of (a)-(c).

Embodiment I-38. The Class 2, Type V CRISPR protein of embodiment I-36 or I-37, comprising a modification at one or more amino acid positions in the NTSB domain relative to SEQ ID NO: 145 selected from the group consisting of P2, S4, Q9, E15, G20, G33, L41, Y51, F55, L68, A70, E75, K88, and G90.

Embodiment I-39. The Class 2, Type V CRISPR protein of embodiment I-38, wherein the one or more modifications at one or more amino acid positions in the NTSB domain are selected from the group consisting of an insertion of G at position 2, an insertion of I at position 4, an insertion of L at position 4, Q9P, E15S, G20D, a deletion of S at position 30, G33T, L41A, Y51T, F55V, L68D, L68E, L68K, A70Y, A70S, E75A, E75D, E75P, K88Q, and G90Q relative to SEQ ID NO: 145.

Embodiment I-40. The Class 2, Type V CRISPR protein of any one of embodiments I-36 to I-39, comprising a modification at one or more amino acid positions in the helical I-II domain relative to SEQ ID NO: 163 selected from the group consisting of 124, A25, Y29 G32, G44, S48, S51, Q54, I56, V63, S73, L74, K97, V100, M112, L116, G137, F138, and S140.

Embodiment I-41. The Class 2, Type V CRISPR protein of embodiment I-40, wherein the one or more modifications at one or more amino acid positions in the helical I-II domain are selected from the group consisting of an insertion of T at position 24, an insertion of C at position 25, Y29F, G32Y, G32N, G32H, G32S, G32T, G32A, G32V, a deletion of G at position 32, G32S, G32T, G44L, G44H, S48H, S48T, S51T, Q54H, I56T, V63T, S73H, L74Y, K97G, K97S, K97D, K97E, V100L, M112T, M112W, M112R, M112K, L116K, G137R, G137K, G137N, an insertion of Q at position 138, and S140Q relative to SEQ ID NO: 163.

Embodiment I-42. The Class 2, Type V CRISPR protein of any one of embodiments I-36 to I-41, comprising a modification at one or more amino acid positions in the helical II domain relative to SEQ ID NO: 161 selected from the group consisting of L2, V3, E4, R5, Q6, A7, E9, V10, D11, W12, W13, D14, M15, V16, C17, N18, V19, K20, L22, I23, E25, K26, K31, Q35, L37, A38, K41, R 42, Q43, E44, L46, K57, Y65, G68, L70, L71, L72, E75, G79, D81, W82, K84, V85, Y86, D87, I93, K95, K96, E98, L100, K102, I104, K105, E109, R110, D114, K118, A120, L121, W124, L125, R126, A127, A129, I133, E134, G135, L136, E138, D140, K141, D142, E143, F144, C145, C147, E148, L149, K150, L151, Q152, K153, L158, E166, and A167.

Embodiment I-43. The Class 2, Type V CRISPR protein of embodiment I-42, wherein the one or more modifications at one or more amino acid positions in the helical II domain are selected from the group consisting of an insertion of A at position 2, an insertion of H at position 2, a deletion of L at position 2 and a deletion of V at position 3, V3E, V3Q, V3F, a deletion of V at position 3, an insertion of D at position 3, V3P, E4P, a deletion of E at position 4, E4D, E4L, E4R, R5N, Q6V, an insertion of Q at position 6, an insertion of G at position 7, an insertion of H at position 9, an insertion of A at position 9, VD10, an insertion of T1 at position 0, a deletion of V at position 10, an insertion of F at position 10, an insertion of D at position 11, a deletion of D at position 11, D11 S, a deletion of W at position 12, W12T, W12H, an insertion of P at position 12, an insertion of Q at position 13, an insertion of G at position 12, an insertion of R at position 13, W13P, W13D, an insertion of D at position 13, W13L, an insertion of P at position 14, an insertion of D at position 14, a deletion of D at position 14 and a deletion of M at position 15, a deletion of M at position 15, an insertion of T at position 16, an insertion of P at position 17, N18I, V19N, V19H, K20D, L22D, 123S, E25C, E25P, an insertion of G at position 25, K26T, K27E, K31L, K31Y, Q35D, Q35P, an insertion of S at position 37, a deletion of L at position 37 and a deletion of A at position 38, K41L, an insertion of R at position 42, a deletion of Q at position 43 and a deletion of E at position 44, L46N, K57Q, Y65T, G68M, L70V, L71C, L72D, L72N, L72W, L72Y, E75F, E75L, E75Y, G79P, an insertion of E at position 79, an insertion of T at position 81, an insertion of R at position 81, an insertion of W at position 81, an insertion of Y at position 81, an insertion of W at position 82, an insertion of Y at position 82, W82G, W82R, K84D, K84H, K84P, K84T, V85L, V85A, an insertion of L at position 85, Y86C, D87G, D87M, D87P, I93C, K95T, K96R, E98G, L100A, K102H, 1104T, I104S, 1104Q, K105D, an insertion of K at position 109, E109L, R110D, a deletion of R at position 110, DJ14E, an insertion of D at position 114, K118P, A120R, L121T, W124L, L125C, R126D, A127E, A127L, A129T, A129K, I133E, an insertion of C at position 133, an insertion of S at position 134, an insertion of G at position 134, an insertion of R at position 135, G135P, L136K, L136D, L136S, L136H, a deletion of E at position 138, D140R, an insertion of D at position 140, an insertion of P at position 141, an insertion of D at position 142, a deletion of E at position 143+a deletion of F at position 144, an insertion of Q at position 143, F144K, a deletion of F at position 144, a deletion of F at position 144 and a deletion of C at position 145, C145R, an insertion of G at position 145, C145K, C147D, an insertion of V at position 148, E148D, an insertion of H at position 149, L149R, K150R, L151H, Q152C, K153P, L158S, E166L, and an insertion of F at position 167 relative to SEQ ID NO: 161.

Embodiment I-44. The Class 2, Type V CRISPR protein of any one of embodiments I-36 to I-43, comprising a modification at one or more amino acid positions in the RuvC-I domain relative to SEQ ID NO: 162 selected from the group consisting of 14, K5, P6, M7, N8, L9, V12, G49, K63, K80, N83, R90, M125, and L146.

Embodiment I-45. The Class 2, Type V CRISPR protein of embodiment I-44, wherein the one or more modifications at one or more amino acid positions in the RuvC-I domain are selected from the group consisting of an insertion of I at position 4, an insertion of S at position 5, an insertion of T at position 6, an insertion of N at position 6, an insertion of R at position 7, an insertion of K at position 7, an insertion of H at position 8, an insertion of S at position 8, V12L, G49W, G49R, S51R, S51K, K62S, K62T, K62E, V65A, K80E, N83G, R90H, R90G, M125S, M125A, L137Y, an insertion of P at position 137, a deletion of L at position 141, L141R, L141D, an insertion of Q at position 142, an insertion of R at position 143, an insertion of N at position 143, E144N, an insertion of P at position 146, L146F, P147A, K149Q, T150V, an insertion of R at position 152, an insertion of H153, T155Q, an insertion of H at position 155, an insertion of R at position 155, an insertion of L at position 156, a deletion of L at position 156, an insertion of W at position 156, an insertion of A at position 157, an insertion of F at position 157, A157S, Q158K, a deletion of Y at position 159, T160Y, T160F, an insertion of I at position 161, S161P, T163P, an insertion of N at position 163, C164K, and C164M relative to SEQ ID NO: 161.

Embodiment I-46. The Class 2, Type V CRISPR protein of any one of embodiments I-36 to I-45, comprising a modification at one or more amino acid positions in the OBD-I domain relative to SEQ ID NO: 152 selected from the group consisting of I3, K4, R5, I6, N7, K8, K15, D16, N18, P27, M28, V33, R34, M36, R41, L47, R48, E52, P55, and Q56.

Embodiment I-47. The Class 2, Type V CRISPR protein of embodiment I-46, wherein the one or more modifications at one or more amino acid positions in the OBD-J domain are selected from the group consisting of an insertion of G at position 3, I3G, 13E, an insertion of G at position 4, K4G, K4P, K4S, K4W, K4W, R5P, an insertion ofP at position 5, an insertion of G at position 5, RSS, an insertion of S at position 5, R5A, R5P, R5G, R5L, 16A, 16L, an insertion of G at position 6, N7Q, N7L, N7S, K8G, K15F, D16W, an insertion of F at position 16, an insertion of F18, an insertion of P at position 27, M28P, M28H, V33T, R34P, M36Y, R41P, L47P, an insertion of P at position 48, E52P, an insertion of P at position 55, a deletion of P at position 55 and a deletion of Q at position 56, Q56S, Q56P, an insertion of D at position 56, an insertion of T at position 56, and Q56P relative to SEQ ID NO: 152.

Embodiment I-48. The Class 2, Type V CRISPR protein of any one of embodiments I-36 to I-47, comprising a modification at one or more amino acid positions in the OBD-II domain relative to SEQ ID NO: 164 selected from the group consisting of S2, I3, L4, K11, V24, K37, R42, A53, T58, K63, M70, I82, Q92, G93, K110, L121, R124, R141, E143, V144, and L145.

Embodiment I-49. The Class 2, Type V CRISPR protein of embodiment I-48, wherein the one or more modifications at one or more amino acid positions in the OBD-II domain are selected from the group consisting of a deletion of S at position 2, I3R, I3K, a deletion of I at position 3 and a deletion of L4, a deletion of L at position 4, K11T, an insertion of P at position 24, K37G, R42E, an insertion of S at position 53, an insertion of R at position 58, a deletion of K at position 63, M70T, 182T, Q92I, Q92F, Q92V, Q92A, an insertion of A at position 93, K110Q, R115Q, L121T, an insertion of A at position 124, an insertion of R at position 141, an insertion of D at position 143, an insertion of A at position 143, an insertion of W at position 144, and an insertion of A at position 145 relative to SEQ ID NO: 152.

Embodiment I-50. The Class 2, Type V CRISPR protein of any one of embodiments I-36 to I-49, comprising a modification at one or more amino acid positions in the TSL domain relative to SEQ ID NO: 159 selected from the group consisting of S1, N2, C3, G4, F5, I7, K18, V58, S67, T76, G78, S80, G81, E82, S85, V96, and E98.

Embodiment I-51. The Class 2, Type V CRISPR protein of embodiment I-50, wherein the one or more modifications at one or more amino acid positions in the OBD-II domain are selected from the group consisting of an insertion of M at position 1, a deletion of N at position 2, an insertion of V at position 2, C3S, an insertion of G at position 4, an insertion of W at position 4, F5P, an insertion of W at position 7, K18G, V58D, an insertion of A at position 67, T76E, T76D, T76N, G78D, a deletion of S at position 80, a deletion of G at position 81, an insertion of E at position 82, an insertion of N at position 82, S85I, V96C, V96T, and E98D relative to SEQ ID NO: 159.

Embodiment I-52. The Class 2, Type V CRISPR protein of any one of embodiments I-28 to I-51, exhibiting an improved characteristic relative to SEQ ID NO: 361, wherein the improved characteristic comprises increased binding affinity to a gRNA, increased binding affinity to the target nucleic acid, improved ability to utilize a greater spectrum of PAM sequences in the editing of the target nucleic acid, improved unwinding of the target nucleic acid, increased editing activity, improved editing efficiency, improved editing specificity for cleavage of the target nucleic acid, decreased off-target editing or cleavage of the target nucleic acid, increased percentage of a eukaryotic genome that can be edited, increased activity of the nuclease, increased target strand loading for double strand cleavage, decreased target strand loading for single strand nicking, increased binding of the non-target strand of DNA, improved protein stability, increased protein:gRNA (RNP) complex stability, and improved fusion characteristics.

Embodiment I-53. The Class 2, Type V CRISPR protein of embodiment I-52, wherein the improved characteristic comprises increased cleavage activity at a target nucleic sequence comprising an TTC, ATC, GTC, or CTC PAM sequence.

Embodiment I-54. The Class 2, Type V CRISPR protein of embodiment I-53, wherein the improved characteristic comprises increased cleavage activity at a target nucleic acid sequence comprising an ATC or CTC PAM sequence relative to cleavage activity of the sequence of SEQ ID NO: 361.

Embodiment I-55. The Class 2, Type V CRISPR protein of embodiment I-54, wherein the improved cleavage activity is an enrichment score (log 2) of at least about 1.5, at least about 2.0, at least about 2.5, at least about 3, at least about 3.5, at least about 4, at least about 4.5, at least about 5, at least about 6, at least about 7, at least about 8 or more greater compared to score of the sequence of SEQ ID NO: 361 in an in vitro assay.

Embodiment I-56. The Class 2, Type V CRISPR protein of embodiment I-54, wherein the improved characteristic comprises increased cleavage activity at a target nucleic acid sequence comprising an CTC PAM sequence relative to the sequence of SEQ ID NO: 361.

Embodiment I-57. The Class 2, Type V CRISPR protein of embodiment I-56, wherein the improved cleavage activity is an enrichment score (log 2) of at least about 2, at least about 2.5, at least about 3, at least about 3.5, at least about 4, at least about 4.5, at least about 5, or at least about 6 or more greater compared to the score of the sequence of SEQ ID NO: 361 in an in vitro assay.

Embodiment I-58. The Class 2, Type V CRISPR protein of embodiment I-53, wherein the improved characteristic comprises increased cleavage activity at a target nucleic acid sequence comprising an TTC PAM sequence relative to the sequence of SEQ ID NO: 361.

Embodiment I-59. The Class 2, Type V CRISPR protein of embodiment I-58, wherein the improved cleavage activity is an enrichment score of at least about 1.5, at least about 2.0, at least about 2.5, at least about 3, at least about 3.5, at least about 4, at least about 4.5, at least about 5, or at least about 6 log 2 or more greater compared to the sequence of SEQ ID NO: 361 in an in vitro assay.

Embodiment I-60. The Class 2, Type V CRISPR protein of embodiment I-52, wherein the improved characteristic comprises increased specificity for cleavage of the target nucleic acid sequence relative to the sequence of SEQ ID NO:361.

Embodiment I-61. The Class 2, Type V CRISPR protein of embodiment I-60, wherein the increased specificity is an enrichment score of at least about 2.0, at least about 2.5, at least about 3, at least about 3.5, at least about 4, at least about 4.5, at least about 5, or at least about 6 log 2 or more greater compared to the sequence of SEQ ID NO: 361 in an in vitro assay.

Embodiment I-62. The Class 2, Type V CRISPR protein of embodiment I-52, wherein the improved characteristic comprises decreased off-target cleavage of the target nucleic acid sequence.

Embodiment I-63. The Class 2, Type V CRISPR protein of any one of embodiments I-28 to I-62, wherein the Class 2, Type V CRISPR protein has a sequence selected from the group consisting of the sequences of SEQ ID NOS: 95-142, as set forth in Table 3, or a sequence having at least about 50%, at least about 60%, at least about 70%, at least about 80%, at least about 90%, or at least about 95%, or at least about 96%, or at least about 97%, or at least about 98%, or at least about 99%, sequence identity thereto.

Embodiment I-64. The Class 2, Type V CRISPR protein of any one of embodiments I-28 to I-62, comprising a sequence selected from the group consisting of SEQ ID NOS: 95-142, as set forth in Table 3.

Embodiment I-65. The Class 2, Type V CRISPR protein of any one of embodiments I-28 to I-64, comprising one or more nuclear localization signals (NLS).

Embodiment I-66. The Class 2, Type V CRISPR protein of embodiment I-65, wherein the one or more NLS are selected from the group of sequences consisting of PKKKRKV (SEQ ID NO: 189), KRPAATKKAGQAKKKK (SEQ ID NO: 190), PAAKRVKLD (SEQ ID NO: 191), RQRRNELKRSP (SEQ ID NO: 192), NQSSNFGPMKGGNFGGRSSGPYGGGGQYFAKPRNQGGY (SEQ ID NO: 193), RMRIZFKNKGKDTAELRRRRVEVSVELRKAKKDEQILKRRNV (SEQ ID NO: 194), VSRKRPRP (SEQ ID NO: 195), PPKKARED (SEQ ID NO: (196), PQPKKKPL (SEQ ID NO: 197), SALIKKKKKMAP (SEQ ID NO: 198), DRLRR (SEQ ID NO: 199), PKQKKRK (SEQ ID NO: 200), RKLKKKIKKL (SEQ ID NO: 201), REKKKFLKRR (SEQ ID NO: 202), KRKGDEVDGVDEVAKKKSKK (SEQ ID NO: 203), RKCLQAGMNLEARKTKK (SEQ ID NO: 204), PRPRKIPR (SEQ ID NO: 205), PPRKKRTVV (SEQ ID NO: 2206), NLSKKKKRKREK (SEQ ID NO: 207), RRPSRPFRKP (SEQ ID NO: 208), KRPRSPSS (SEQ ID NO: 209), KRGINDRNFWRGENERKTR (SEQ ID NO: 210), PRPPKMARYDN (SEQ ID NO: 211), KRSFSKAF (SEQ ID NO: 212), KLKIKRPVK (SEQ ID NO: 213), PKKKRKVPPPPAAKRVKLD (SEQ ID NO: 214), PKTRRRPRRSQRKRPPT (SEQ ID NO: 215), SRRRKANPTKLSENAKKLAKEVEN (SEQ ID NO: 216), KTRRRPRRSQRKRPPT (SEQ ID NO: 217), RRKKRRPRRKKRR (SEQ ID NO: 218), PKKKSRKPKKKSRK (SEQ ID NO: 219), HKKKHPDASVNFSEFSK (SEQ ID NO: 220), QRPGPYDRPQRPGPYDRP (SEQ ID NO: 221), LSPSLSPLLSPSLSPL (SEQ ID NO: 222), RGKGGKGLGKGGAKRHRK (SEQ ID NO: 223), PKRGRGRPKRGRGR (SEQ ID NO: 224), PKKKRKVPPPPKKKRKV (SEQ ID NO: 226), PAKRARRGYKC (SEQ ID NO: 227), KLGPRKATGRW (SEQ ID NO: 228), PRRKREE (SEQ ID NO: 229), PYRGRKE (SEQ ID NO: 230), PLRKRPRR (SEQ ID NO: 231), PLRKRPRRGSPLRKRPRR (SEQ ID NO: 232), PAAKRVKLDGGKRTADGSEFESPKKKRKV (SEQ ID NO: 233), PAAKRVKLDGGKRTADGSEFESPKKKRKVGIHGVPAA (SEQ ID NO: 234), PAAKRVKLDGGKRTADGSEFESPKKKRKVAEAAAKEAAAKEAAAKA (SEQ ID NO: 235), PAAKRVKLDGGKRTADGSEFESPKKKRKVPG (SEQ ID NO: 236), KRKGSPERGERKRHW (SEQ ID NO: 237), KRTADSQHSTPPKTKRKVEFEPKKKRKV (SEQ ID NO: 238), and PKKKRKVGGSKRTADSQHSTPPKTKRKVEFEPKKKRKV (SEQ ID NO: 239), and, optionally, wherein the one or more NLS are linked to the Class 2, Type V CRISPR protein or to an adjacent NLS with a linker peptide wherein the linker peptide is selected from the group consisting of SR, RS, (G)n (SEQ ID NO: 240), (GS)n (SEQ ID NO: 241), (GSGGS)n (SEQ ID NO: 242), (GGSGGS)n (SEQ ID NO: 243), (GGGS)n (SEQ ID NO: 244), GGSG (SEQ ID NO: 245), GGSGG (SEQ ID NO: 246), GSGSG (SEQ ID NO: 247), GSGGG (SEQ ID NO: 248), GGGSG (SEQ ID NO: 249), GSSSG (SEQ ID NO: 250), GPGP (SEQ ID NO: 251), GGP, PPP, PPAPPA (SEQ ID NO: 252), PPPG (SEQ ID NO: 253), PPPGPPP (SEQ ID NO: 254), PPP(GGGS)n (SEQ ID NO: 255), (GGGS)nPPP (SEQ ID NO: 256), AEAAAKEAAAKEAAAKA (SEQ ID NO: 257), and TPPKTKRKVEFE (SEQ ID NO: 258), wherein n is 1 to 5.

Embodiment I-67. The Class 2, Type V CRISPR protein of embodiment I-65 or I-66, wherein the one or more NLS are positioned at or near the C-terminus of the protein.

Embodiment I-68. The Class 2, Type V CRISPR protein of embodiment I-65 or I-66, wherein the one or more NLS are positioned at or near at the N-terminus of the protein.

Embodiment I-69. The Class 2, Type V CRISPR protein of embodiment I-65 or I-66, comprising at least two NLS, wherein the at least two NLS are positioned at or near the N-terminus and at or near the C-terminus of the protein.

Embodiment I-70. The Class 2, Type V CRISPR protein of any one of embodiments I-28 to I-69, wherein the Class 2, Type V CRISPR protein is capable of forming a ribonuclear protein complex (RNP) with a gRNA.

Embodiment I-71. The Class 2, Type V CRISPR protein of embodiment I-70, wherein the RNP exhibits at least one or more improved characteristics as compared to a an RNP of a reference protein of any one of SEQ ID NOS: 358, 359, or 361 and a gRNA of SEQ ID NO: 17 or SEQ ID NO: 18.

Embodiment I-72. The Class 2, Type V CRISPR protein of embodiment I-71, wherein the improved characteristic is selected from the group consisting of increased binding affinity to a guide nucleic acid (gRNA); increased binding affinity to a target nucleic acid; improved ability to utilize a greater spectrum of one or more PAM sequences, including ATC, CTC, GTC, or TTC, in the editing of target nucleic acid; increased unwinding of the target nucleic acid; increased editing activity; increased editing efficiency; increased editing specificity of the target nucleic acid; increased nuclease activity; increased target strand loading for double strand cleavage; decreased target strand loading for single strand nicking; decreased off-target cleavage of the target nucleic acid; increased binding of non-target nucleic acid strand; and increased protein:gRNA complex (RNP) stability.

Embodiment I-73. The Class 2, Type V CRISPR protein of embodiment I-71 or I-72, wherein the improved characteristic of the RNP is at least about 1.1 to about 100,000-fold increased relative to an RNP comprising any off SEQ ID NOS: 358, 359, or 361.

Embodiment I-74. The Class 2, Type V CRISPR protein of embodiment I-71 or I-72, wherein the improved characteristic of the RNP is at least about 10-fold, at least about 100-fold, at least about 1,000-fold, or at least about 10,000-fold increased relative to an RNP comprising any of SEQ ID NOS: 358, 350 or 361.

Embodiment I-75. The Class 2, Type V CRISPR protein of any one of embodiments I-71 to I-74, wherein the improved characteristic of the RNP comprises a 1.1 to 100-fold improvement in editing efficiency compared to the RNP of an RNP comprising SEQ ID NOS: 358, 350 or 361.

Embodiment I-76. A gene editing pair comprising a gRNA and a Class 2, Type V CRISPR protein, the pair comprising:
a. a gRNA of any one of embodiments I-24 to I-27; and
b. a Class 2, Type V CRISPR protein of any one of embodiments I-28 to I-75.

Embodiment I-77. The gene editing pair of embodiment I-76, wherein the gRNA and the Class 2, Type V CRISPR protein are capable of forming a ribonuclear protein complex (RNP).

Embodiment I-78. The gene editing pair of embodiment I-76 or I-77, wherein the gRNA and the Class 2, Type V CRISPR protein are associated together as a ribonuclear protein complex (RNP).

Embodiment I-79. The gene editing pair of embodiment I-77 or I-78, wherein an RNP of the Class 2, Type V CRISPR protein and the gRNA exhibit at least one or more improved characteristics as compared to an RNP comprising any of SEQ ID NOS: 358, 359, or 361, and a gRNA comprising SEQ ID NO: 17 or SEQ ID NO: 18.

Embodiment I-80. The gene editing pair of embodiment I-79, wherein the improved characteristic is selected from one or more of the group consisting of increased binding affinity of the Class 2, Type V CRISPR protein to the gRNA; increased binding affinity to a target nucleic acid; increased ability to utilize a greater spectrum of one or more PAM sequences, including ATC, CTC, GTC, or TTC, in the editing of target nucleic acid; increased unwinding of the target nucleic acid; increased editing activity; increased editing efficiency; increased editing specificity of the target nucleic acid; increased nuclease activity; increased target strand loading for double strand cleavage; decreased target strand loading for single strand nicking; decreased off-target cleavage of the target nucleic acid; increased binding of non-target nucleic acid strand; increased protein:gRNA complex (RNP) stability; and increased fusion characteristics.

Embodiment I-81. The gene editing pair of embodiment I-79 or I-80, wherein the improved characteristic of the RNP of the Class 2, Type V CRISPR protein and the gRNA is at least about 1.1 to about 100-fold or more increased relative to the RNP of an earlier generation CasX and gRNA gene editing pair in a comparable in vitro assay system.

Embodiment I-82. The gene editing pair of embodiment I-79 or I-80, wherein the improved characteristic of the Class 2, Type V CRISPR protein is at least about 1.1, at least about 2, at least about 10, at least about 100-fold or more increased relative to an earlier generation CasX and gRNA gene editing pair in a comparable in vitro assay system.

Embodiment I-83. The gene editing pair of any one of embodiments I-77 to I-82, wherein the RNP comprising the Class 2, Type V CRISPR protein and the gRNA exhibits greater editing efficiency and/or binding of a target nucleic acid sequence in the target nucleic acid when any one of the PAM sequences TTC, ATC, GTC, or CTC is located 1 nucleotide 5′ to the non-target strand of the protospacer having identity with the targeting sequence of the gRNA in a cellular assay system compared to the editing efficiency and/or binding of an RNP comprising an earlier generation CasX protein and a reference gRNA in a comparable assay system.

Embodiment I-84. The gene editing pair of embodiment I-83, wherein the PAM sequence is TTC.

Embodiment I-85. The gene editing pair of embodiment I-83, wherein the PAM sequence is ATC.

Embodiment I-86. The gene editing pair of embodiment I-83, wherein the PAM sequence is CTC.

Embodiment I-87. The gene editing pair of embodiment I-83, wherein the PAM sequence is GTC.

Embodiment I-88. The gene editing pair of any one of embodiments I-83 to I-87, wherein the RNP comprising the Class 2, Type V CRISPR and the gRNA exhibits increased binding affinity for the one or more PAM sequences that is at least 1.5-fold, at least 2-fold, at least 4-fold, at least 10-fold, at least 20-fold, at least 30-fold, or at least 40-fold greater compared to the binding affinity of an RNP of any one earlier generation CasX and gRNA gene editing pairs, when assessed in a comparable in vitro assay system.

Embodiment I-89. The gene editing pair of any one of embodiments I-77 to I-88, wherein the RNP of the Class 2, Type V CRISPR protein and the gRNA exhibits increased editing efficiency that is at least 1.5-fold, at least 2-fold, at least 4-fold, at least 10-fold, at least 20-fold, at least 30-fold, or at least 40-fold greater compared to the editing efficiency of an RNP of any one of the reference proteins of earlier generation CasX and gRNA gene editing pairs, when assessed in a comparable in vitro assay system.

Embodiment I-90. The gene editing pair of any one of embodiments I-77 to I-89, wherein the Class 2, Type V CRISPR and the gRNA are able to form RNP having at least about a 5%, at least about a 10%, at least about a 15%, or at least about a 20% higher percentage of cleavage-competent conformation compared to an RNP of any one of the reference proteins of earlier generation CasX and gRNA gene editing pairs, when assessed in a comparable in vitro assay system.

Embodiment I-91. The gene editing pair of any one of embodiments I-77 to I-90, wherein the RNP comprising the Class 2, Type V CRISPR and the gRNA exhibit a cleavage rate for the target nucleic acid in a timed in vitro assay that is at least about 5-fold, at least about 10-fold, or at least about 20-fold higher compared to an RNP of any one earlier generation CasX and gRNA gene editing pairs, when assessed in a comparable in vitro assay system.

Embodiment I-92. The gene editing pair of any one of embodiments I-77 to I-91, wherein the RNP comprising the Class 2, Type V CRISPR and the gRNA exhibit higher percent editing of the target nucleic acid in a timed in vitro assay that is at least about 5-fold, at least about 10-fold, at least about 20-fold, or at least about 100-fold higher compared to an RNP of any one earlier generation CasX and gRNA gene editing pairs, when assessed in a comparable in vitro assay system.

Embodiment I-93. A catalytically-dead Class 2, Type V CRISPR protein, comprising a sequence derived from any of SEQ ID NOS: 95-142 or a sequence having at least 60%, at least 70%, at least 80%, at least 90%, at least 95%, at least 98%, at least 99% thereto.

Embodiment I-94. A catalytically-dead Class 2, Type V CRISPR protein, comprising a sequence derived from any of SEQ ID NOS: 95-142.

Embodiment I-95. The Class 2, Type V CRISPR protein of embodiment I-93 or I-94, wherein and RNP of the catalytically-dead Class 2, Type V CRISPR protein and a gRNA of any one of embodiments I-24 to I-27 retain the ability to bind target nucleic acid.

Embodiment I-96. A nucleic acid comprising a sequence that encodes the gRNA scaffold of any one of embodiments I-1 to I-23, or the gRNA of any one of embodiments I-24 to 1-27.

Embodiment I-97. A nucleic acid comprising a sequence that encodes the Class 2, Type V CRISPR protein of any one of embodiments I-28 to I-75.

Embodiment I-98. The nucleic acid of embodiment I-97, wherein the sequence that encodes the Class 2, Type V CRISPR protein is codon optimized for expression in a eukaryotic cell.

Embodiment I-99. A vector comprising the gRNA of any one of embodiments I-24 to I-27, the Class 2, Type V CRISPR protein of any one of embodiments I-28 to I-75, or the nucleic acid of any one of embodiments I-96 to I-98.

Embodiment I-100. The vector of embodiment I-99, wherein the vector comprises a promoter.

Embodiment I-101. The vector of embodiment I-99 or I-100, wherein the vector is selected from the group consisting of a retroviral vector, a lentiviral vector, an adenoviral vector, an adeno-associated viral (AAV) vector, a herpes simplex virus (HSV) vector, a CasX delivery particle (XDP), a plasmid, a minicircle, a nanoplasmid, a DNA vector, and an RNA vector.

Embodiment I-102. The vector of embodiment I-101, wherein the vector is an AAV vector.

Embodiment I-103. The vector of embodiment I-102, wherein the AAV vector is selected from AAV1, AAV2, AAV3, AAV4, AAV5, AAV6, AAV7, AAV8, AAV9, AAV10, AAV11, AAV12, AAV-Rh74, or AAVRh10.

Embodiment I-104. The vector of embodiment I-101, wherein the vector is a retroviral vector.

Embodiment I-105. The vector of embodiment I-101, wherein the vector is a XDP comprising one or more components of a gag polyprotein.

Embodiment I-106. The vector of embodiment I-105, wherein the one or more components of the gag polyprotein are selected from the group consisting of matrix protein (MA), a nucleocapsid protein (NC), a capsid protein (CA), a p1 peptide, a p6 peptide, a P2A peptide, a P2B peptide, a P10 peptide, a p12 peptide, a PP21/24 peptide, a P12/P3/P8 peptide, a P20 peptide, and a protease cleavage site.

Embodiment I-107. The vector of embodiment I-105 or I-106, wherein the Class 2, Type V CRISPR protein and the gRNA are associated together in an RNP.

Embodiment I-108. The vector of any one of embodiments I-105 to I-107, comprising a glycoprotein tropism factor.

Embodiment I-109. The vector of embodiment I-108, wherein the glycoprotein tropism factor has binding affinity for a cell surface marker of a target cell and facilitates entry of the XDP into the target cell.

Embodiment I-110. The vector of any one of embodiments I-99 to I-109, comprising the donor template.

Embodiment I-111. A host cell comprising the vector of any one of embodiments I-99 to I-110.

Embodiment I-112. The host cell of embodiment I-111, wherein the host cell is selected from the group consisting of Baby Hamster Kidney fibroblast (BHK) cells, human embryonic kidney 293 (HEK293) cells, human embryonic kidney 293T (HEK293T) cells, NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS) cells, HeLa, Chinese hamster ovary (CHO) cells, or yeast cells.

Embodiment I-113. A method of modifying a target nucleic acid in a cell, comprising contacting the target nucleic acid of the cell with: i) the gene editing pair of any one of embodiments I-76 to I-92; ii) the gene editing pair of any one of embodiments I-76 to I-92 together with a donor template; iii) one or more nucleic acids encoding the gene editing pair of (i) or (ii); iv) a vector comprising the nucleic acid of (iii); v) an XDP comprising the gene editing pair of (i) or (ii); or vi) combinations of two or more of (i) to (v), wherein the contacting of the target nucleic acid modifies the target nucleic acid.

Embodiment I-114. The method of embodiment I-113, comprising contacting the target with a plurality of gene editing pairs comprising a first and a second, or a plurality of gRNAs comprising targeting sequences complementary to different or overlapping regions of the target nucleic acid.

Embodiment I-115. The method of embodiment I-113, comprising contacting the target with a plurality of nucleic acids encoding gene editing pairs comprising a first and a second, or a plurality of gRNAs comprising targeting sequences complementary to different or overlapping regions of the target nucleic acid.

Embodiment I-116. The method of embodiment I-113, comprising contacting the target with a plurality of XDP comprising gene editing pairs comprising a first and a second, or a plurality of gRNAs comprising targeting sequences complementary to different or overlapping regions of the target nucleic acid.

Embodiment I-117. The method of any one of embodiment I-113, wherein the contacting comprises binding the target nucleic acid with the gene editing pair and introducing one or more single-stranded breaks in the target nucleic acid, wherein the modifying comprises introducing a mutation, an insertion, or a deletion in the target nucleic acid.

Embodiment I-118. The method of any one of embodiments I-113 to I-116 wherein the contacting comprises binding the target nucleic acid and introducing one or more double-stranded breaks in the target nucleic acid, wherein the modifying comprises introducing a mutation, an insertion, or a deletion in the target nucleic acid.

Embodiment I-119. The method of any one of embodiments I-113 to I-118, comprising contacting the target nucleic acid with a nucleotide sequence of a donor template nucleic acid wherein the donor template comprises a nucleotide sequence having homology to the target nucleic acid.

Embodiment I-120. The method of embodiment I-119, wherein the donor template comprises homologous arms on the 5′ and 3′ ends of the donor template.

Embodiment I-121. The method of embodiment I-119 or I-120, wherein the donor template is inserted in the target nucleic acid at the break site by homology-directed repair.

Embodiment I-122. The method of embodiment I-121, wherein the donor template is inserted in the target nucleic acid at the break site by non-homologous end joining (NIHEJ) or micro-homology end joining (MMEJ).

Embodiment I-123. The method of any one of embodiments I-113 to I-122, wherein the modifying of the cell occurs in vitro.

Embodiment I-124. The method of any one of embodiments I-113 to I-122, wherein modifying of the cell occurs in vivo.

Embodiment I-125. The method of any one of embodiments I-113 to I-124, wherein the cell is a eukaryotic cell.

Embodiment I-126. The method of embodiment I-125, wherein the eukaryotic cell is selected from the group consisting of a rodent cell, a mouse cell, a rat cell, a primate cell, and a non-human primate cell.

Embodiment I-127. The method of embodiment I-125, wherein the eukaryotic cell is a human cell.

Embodiment I-128. The method of any one of embodiments I-113 to I-127, wherein the cell is selected from the group consisting of an embryonic stem cell, an induced pluripotent stem cell, a germ cell, a fibroblast, an oligodendrocyte, a glial cell, a hematopoietic stem cell, a neuron progenitor cell, a neuron, a muscle cell, a bone cell, a hepatocyte, a pancreatic cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, a parenchymal cell, an epithelial cell, an endothelial cell, a mesothelial cell, a fibroblast cell, an osteoblast cell, a chondrocyte cell, an exogenous cell, an endogenous cell, a stem cell, a hematopoietic stem cell, a bone-marrow derived progenitor cell, a myocardial cell, a skeletal cell, a fetal cell, an undifferentiated cell, a multi-potent progenitor cell, a unipotent progenitor cell, a monocyte, a cardiac myoblast, a skeletal myoblast, a macrophage, a capillary endothelial cell, a xenogenic cell, an allogenic cell, an autologous cell, and a post-natal stem cell.

Embodiment I-129. The method of any one of embodiments I-124 to I-128, wherein the cell is in a subject.

Embodiment I-130. The method of embodiment I-129, wherein the modifying occurs in the cells of the subject having a mutation in an allele of a gene wherein the mutation causes a disease or disorder in the subject.

Embodiment I-131. The method of embodiment I-130, wherein the modifying changes the mutation to a wild type allele of the gene or results in the expression of a functional gene product.

Embodiment I-132. The method of embodiment I-130, wherein the modifying knocks down or knocks out the allele of the gene causing the disease or disorder in the subject.

Embodiment I-133. The method of any one of embodiments I-129 to I-132, wherein the cell is autologous with respect to the subject.

Embodiment I-134. The method of any one of embodiments I-129 to I-132, wherein the cell is allogeneic autologous with respect to the subject.

Embodiment I-135. The method of any one of embodiments I-113 to I-134, wherein the vector is an Adeno-Associated Viral (AAV) vector.

Embodiment I-136. The method of embodiment I-135, wherein the AAV is AAV1, AAV2, AAV3, AAV4, AAV5, AAV6, AAV7, AAV8, AAV9, AAV10, AAV-Rh74, or AAVRh10.

Embodiment I-137. The method of embodiment I-113, wherein the vector is a lentiviral vector.

Embodiment I-138. The method of any one of embodiments I-113 to I-137, wherein the vector is administered to a subject in need using a therapeutically effective dose.

Embodiment I-139. The method of embodiment I-138, wherein the subject is selected from the group consisting of mouse, rat, pig, and non-human primate.

Embodiment I-140. The method of embodiment I-138, wherein the subject is a human.

Embodiment I-141. The method of embodiment I-138 wherein the vector is administered by a route of administration selected from the group consisting of intraparenchymal, intravenous, intra-arterial, intracerebroventricular, intracisternal, intrathecal, intracranial, and intraperitoneal routes wherein the administering method is injection, transfusion, or implantation.

Embodiment I-142. The method of embodiment I-138, wherein the vector is administered to the subject according to a treatment regimen comprising one or more consecutive doses using a therapeutically effective dose of the vector.

Embodiment I-143. The method of embodiment I-142, wherein the therapeutically effective dose is administered to the subject as two or more doses over a period of at least two weeks, or at least one month, or at least two months, or at least three months, or at least four months, or at least five months, or at least six months, or once a year, or every 2 or 3 years.

Embodiment I-144. A cell comprising a target nucleic acid modified by the gene editing pair of any one of embodiments I-76 to I-92.

Embodiment I-145. A cell edited by the method of any one of embodiments I-113 to I-143.

Embodiment I-146. The cell of embodiment I-144 or I-145, wherein the cell is a prokaryotic cell.

Embodiment I-147. The cell of embodiment I-144 or I-145, wherein the cell is a eukaryotic cell.

Embodiment I-148. The cell of embodiment I-147, wherein the eukaryotic cell is selected from the group consisting of a rodent cell, a mouse cell, a rat cell, a primate cell, and a non-human primate cell.

Embodiment I-149. The cell of embodiment I-147, wherein the eukaryotic cell is a human cell.

Embodiment I-150. A composition, comprising the Class 2, Type V CRISPR protein of any one of embodiments I-28 to I-75.

Embodiment I-151. The composition of embodiment I-150, comprising the gRNA of any one of embodiments I-24 to I-27.

Embodiment I-152. The composition of embodiment I-151, wherein the protein and the gRNA are associated together in a ribonuclear protein complex (RNP).

Embodiment I-153. The composition of any one of embodiments I-150 to I-152, comprising a donor template nucleic acid wherein the donor template comprises a nucleotide sequence having homology to a target nucleic acid.

Embodiment I-154. The composition of any one of embodiments I-150 to I-153, comprising a buffer, a nuclease inhibitor, a protease inhibitor, a liposome, a therapeutic agent, a label, a label visualization reagent, or any combination of the foregoing.

Embodiment I-155. A composition, comprising a gRNA scaffold of any one of embodiments I-1 to I-23, or a gRNA of any one of embodiments I-24 to I-27.

Embodiment I-156. The composition of embodiment I-155, comprising the Class 2, Type V CRISPR protein of any one of embodiments I-28 to I-75.

Embodiment I-157. The composition of embodiment I-156, wherein the Class 2, Type V CRISPR protein and the gRNA are associated together in a ribonuclear protein complex (RNP).

Embodiment I-158. The composition of any one of embodiments I-155 to I-157, comprising a donor template nucleic acid wherein the donor template comprises a nucleotide sequence having homology to a target nucleic acid.

Embodiment I-159. The composition of any one of embodiments I-155 to I-158, comprising a buffer, a nuclease inhibitor, a protease inhibitor, a liposome, a therapeutic agent, a label, a label visualization reagent, or any combination of the foregoing.

Embodiment I-160. A composition, comprising the gene editing pair of any one of embodiments I-76 to I-92.

Embodiment I-161. The composition of embodiment I-160, comprising a donor template nucleic acid wherein the donor template comprises a nucleotide sequence having homology to a target nucleic acid.

Embodiment I-162. The composition of embodiment I-160 or I-161, comprising a buffer, a nuclease inhibitor, a protease inhibitor, a liposome, a therapeutic agent, a label, a label visualization reagent, or any combination of the foregoing.

Embodiment I-163. A kit, comprising the Class 2, Type V CRISPR protein of any one of embodiments I-28 to I-75 and a container.

Embodiment I-164. The kit of embodiment I-163, comprising a gRNA scaffold of any one of embodiments I-1 to I-23, or a gRNA of any one of embodiments I-24 to I-27.

Embodiment I-165. The kit of embodiment I-163 or I-164, comprising a donor template nucleic acid wherein the donor template comprises a nucleotide sequence having homology to a target nucleic acid sequence of a target nucleic acid.

Embodiment I-166. The kit of any one of embodiments I-163 to I-165, comprising a buffer, a nuclease inhibitor, a protease inhibitor, a liposome, a therapeutic agent, a label, a label visualization reagent, or any combination of the foregoing.

Embodiment I-167. A kit, comprising a gRNA scaffold of any one of embodiments I-1 to I-23, or a gRNA of any one of embodiments I-24 to I-27.

Embodiment I-168. The kit of embodiment I-167, comprising the Class 2, Type V CRISPR protein of any one of embodiments I-28 to I-75.

Embodiment I-169. The kit of embodiment I-167 or I-168, comprising a donor template nucleic acid wherein the donor template comprises a nucleotide sequence having homology to a target nucleic acid sequence of a target nucleic acid.

Embodiment I-170. The kit of any one of embodiments I-167 to I-169, comprising a buffer, a nuclease inhibitor, a protease inhibitor, a liposome, a therapeutic agent, a label, a label visualization reagent, or any combination of the foregoing.

Embodiment I-171. A kit, comprising the gene editing pair of any one of embodiments I-76 to I-92.

Embodiment I-172. The kit of embodiment I-171, comprising a donor template nucleic acid wherein the donor template comprises a nucleotide sequence having homology to a target nucleic acid.

Embodiment I-173. The kit of embodiment I-171 or I-172, comprising a buffer, a nuclease inhibitor, a protease inhibitor, a liposome, a therapeutic agent, a label, a label visualization reagent, or any combination of the foregoing.

Embodiment I-174. An engineered Class 2, Type V CRISPR protein comprising any one of the sequences listed in Table 3.

Embodiment I-175. A gRNA comprising any one of the gRNA scaffold variant sequences listed in Table 2.

Embodiment I-176. The gRNA of embodiment I-175, wherein one or more uracils (U) of the gRNA scaffold variants of the Table 2 sequences are replaced with thymines (T).

Embodiment I-177. The gRNA of embodiment I-176, comprising a targeting sequence of at least 10 to 30 nucleotides complementary to a target nucleic acid.

Embodiment I-178. The gRNA of embodiment I-177, wherein the targeting sequence has 20 nucleotides.

Embodiment I-179. The gRNA of embodiment I-177, wherein the targeting sequence has 19 nucleotides.

Embodiment I-180. The gRNA of embodiment I-177, wherein the targeting sequence has 18 nucleotides.

Embodiment I-181. The gRNA of embodiment I-177, wherein the targeting sequence has 17 nucleotides.

Embodiment I-182. The gRNA of embodiment I-177, wherein the targeting sequence has 16 nucleotides.

Embodiment I-183. The gRNA of embodiment I-177, wherein the targeting sequence has 15 nucleotides.

Embodiment I-184. A method of treating a disease in a subject in need thereof, comprising administering to the subject a therapeutically effective amount of a composition comprising: (a) an engineered class 2, type V CRISPR protein comprising any of SEQ ID NOS: 95-142 and (b) a gRNA comprising any of SEQ ID NOS: 29-81.

Embodiment I-185. A composition comprising: (a) an engineered class 2, type V CRISPR protein comprising any of SEQ ID NOS: 95-142 and (b) a gRNA comprising any of SEQ ID NOS: 29-81, for use as a medicament for the treatment of a subject having a disease.

Embodiment II-1. An engineered ribonucleic acid scaffold (ERS) comprising a sequence having at least at least 90%, at least 95%, at least 98%, at least 99% sequence identity to one of:

(i)
ACUGGCACUUCUAUCUGAUUACUCUGAGAGCCAUCACCAGCGACUAUGUCGUAUGGGU
AAAGCCGCUUACGGACUUCGGUCCGUAAGAGGCAUCAGAG (variant 221);
or
(ii)
SEQ ID NO: 156
(variant 316);

comprising one or more modifications in the sequence, wherein the one or more modifications result in an improved characteristic compared to unmodified (i) variant 221 or (ii) SEQ ID NO: 156.

Embodiment II-2. The ERS of embodiment II-1, wherein the one or modifications comprise mutations in at least two regions of the ERS, wherein the regions are selected from the group consisting of 5′ terminus, pseudoknot stem, triplex loop, triplex, scaffold stem, and extended stem.

Embodiment II-3. The ERS of embodiment II-2, wherein the mutations in the regions are selected from the group consisting of the mutations of Tables 24A and 24B.

Embodiment II-4. The ERS of any one of embodiments II-1 to II-3, wherein the improved characteristic is one or more functional properties selected from the group consisting of improved binding to a CasX nuclease to form a ribonucleoprotein (RNPs), enhanced folding stability of the ERS, increased transcriptional efficiency, and enhanced editing of a target nucleic acid by an RNP comprising the ERS.

Embodiment II-5. The ERS of any one of embodiments II-1 to II-4, wherein the ERS comprises a sequence selected from the group consisting of SEQ ID NOS: 11568-22227 or 23572-24915, or a sequence having at least at least 90%, at least 95%, at least 98%, or at least 99% sequence identity thereto.

Embodiment II-6. The ERS of any one of embodiments II-1 to II-4, wherein the ERS comprises a sequence selected from the group consisting of SEQ ID NOS: 11568-22227 or 23572-24915.

Embodiment II-7. The ERS of any one of embodiments II-1 to II-6, wherein the ERS comprises one or more heterologous RNA sequences in the extended stem.

Embodiment II-8. The ERS of embodiment II-7, wherein the heterologous RNA is selected from the group consisting of a MS2 hairpin, Qβ hairpin, U1 hairpin II, Uvsx hairpin, and a PP7 stem loop, or sequence variants thereof.

Embodiment II-9. The ERS of embodiment II-7 or II-8, wherein the heterologous RNA is capable of binding a protein, a RNA, a DNA, or a small molecule.

Embodiment II-10. The ERS of any one of embodiments II-1 to II-9, wherein the ERSERS comprises a Rev response element (RRE) or a portion thereof.

Embodiment II-11. The ERS of embodiment II-10, wherein the RRE or portion thereof is selected from the group consisting of Stem IIB of the RRE having sequence UGGGCGCAGCGUCAAUGACGCUGACGGUACA, Stem II-V of the RRE having sequence CAGGAAGCACUAUGGGCGCAGCGUCAAUGACGCUGACGGUACAGGCCAGACAAU UAUUGUCUGGUAUAGUGCAGCAGCAGAACAAUUUGCUGAGGGCUAUUGAGGCGC AACAGCAUCUGUUGCAACUCACAGUCUGGGGCAUCAAGCAGCUCCAGGCAAGAA UCCUG, Stem II of the RRE having sequence GCACUAUGGGCGCAGCGUCAAUGACGCUGACGGUACAGGCCAGACAAUUAUUGU CUGGUAUAGUGC, Rev-binding element (RBE) of Stem IIB having sequence GCUGACGGUACAGGC, and full-length RRE having sequence AGGAGCUUUGUUCCUUGGGUUCUUGGGAGCAGCAGGAAGCACUAUGGGCGCAGC GUCAAUGACGCUGACGGUACAGGCCAGACAAUUAUUGUCUGGUAUAGUGCAGCA GCAGAACAAUUUGCUGAGGGCUAUUGAGGCGCAACAGCAUCUGUUGCAACUCAC AGUCUGGGGCAUCAAGCAGCUCCAGGCAAGAAUCCUGGCUGUGGAAAGAUACCU AAAGGAUCAACAGCUCCU.

Embodiment II-12. The ERS of embodiment II-1, wherein the ERS of SEQ ID NO: SEQ ID NO: 156 comprises one or more chemical modifications to the sequence.

Embodiment II-13. The ERS of embodiment II-12, wherein the chemical modification is addition of a 2′O-methyl group to one or more nucleotides of the sequence.

Embodiment II-14. The ERS of embodiment II-12 or II-13, wherein the chemical modification is substitution of a phosphorothioate bond between two or more nucleotides of the sequence.

Embodiment II-15. The ERS of embodiment II-13 or II-14, wherein the modifications results in reduced susceptibility of the ERS to degradation by cellular RNase compared to an unmodified ERS.

Embodiment II-16. The ERS of any one of embodiments II-12 to II-16, wherein the ERS comprises a sequence selected from the group consisting of SEQ ID NOS: 501-532.

Embodiment II-17. The ERS of any one of embodiments II-1 to II-16, comprising a targeting sequence linked at the 3′ end of the ERS that is complementary to a target nucleic acid sequence.

Embodiment II-18. The ERS of embodiment II-13, wherein the targeting sequence has 15, 16, 17, 18, 19, 20, 21, 22, 23, 24, 25, 26, 27, 28, 29, or 30 nucleotides.

Embodiment II-19. The ERS of embodiment II-18, wherein the targeting sequence has 18, 19, or 20 nucleotides.

Embodiment II-20. The ERS of any one of embodiments II-13 to II-19, wherein the ERS is capable of forming a ribonucleoprotein (RNP) complex with a CasX protein.

Embodiment II-21. An engineered CasX protein, comprising a sequence having two or three mutations in the sequence of SEQ ID NO: 228, wherein the mutations result in an improved characteristic compared to unmodified SEQ ID NO: 228.

Embodiment II-22. The engineered CasX protein of embodiment II-21, wherein the mutations are selected from
a. at least one amino acid substitution;
b. at least one amino acid deletion;
c. at least one amino acid insertion; or
d. any combination of (a)-(c).

Embodiment II-23. The engineered CasX protein of embodiment II-21 or II-22, wherein the mutations are selected from the group consisting of the mutations as set forth in Tables 11-13.

Embodiment II-24. The engineered CasX protein of any one of embodiments II-21 to II-23, comprising a sequence selected from SEQ ID NOS: 27857-49628 as set forth in Table 14, or a sequence having at least at least 90%, at least 95%, at least 98%, at least 99% sequence identity thereto.

Embodiment II-25. The engineered CasX protein of any one of embodiments II-21 to II-24, comprising a sequence selected from SEQ ID NOS: 27857-49628 as set forth in Table 14.

Embodiment II-26. The engineered CasX protein of any one of embodiments II-21 to II-25, wherein the improved characteristic is one or more of improved ability to utilize a greater spectrum of PAM sequences in the editing of target nucleic acid, increased nuclease activity, improved editing efficiency, improved editing specificity for the target nucleic acid, decreased off-target editing, increased percentage of a eukaryotic genome that can be efficiently edited, improved ability to form cleavage-competent RNP with an ERS, and improved stability of an RNP complex.

Embodiment II-27. The engineered CasX protein of embodiment II-25, wherein the improved characteristic comprises increased nuclease activity at a target nucleic sequence comprising an TTC, ATC, GTC, or CTC PAM sequence relative to nuclease activity of the sequence of SEQ ID NO: 228.

Embodiment II-28. The engineered CasX protein of any one of embodiments II-21 to 11-27, comprising one or more nuclear localization signals (NLS) selected from the group consisting of the sequences of SEQ ID NOS: 364-457 as set forth in Table 7, and, optionally, wherein the one or more NLS are linked to the engineered CasX protein or to an adjacent NLS with a linker peptide wherein the linker peptide is selected from the group consisting of SR, RS, (G)n (SEQ ID NO: (GS)n, (GSGGS)n, (GGSGGS)n, (GGGS)n, GGSG, GGSGG, GSGSG, GSGGG, GGGSG, GSSSG, GPGP, GGP, PPP, PPAPPA, PPPG, PPPGPPP, PPP(GGGS)n, (GGGS)nPPP, AEAAAKEAAAKEAAAKA, and TPPKTKRKVEFE, wherein n is 1 to 5.

Embodiment II-29. The engineered CasX protein of embodiment II-28, wherein the one or more NLS are positioned at or near the C-terminus of the protein.

Embodiment II-36. The engineered CasX protein of embodiment II-28, wherein the one or more NLS are positioned at or near at the N-terminus of the protein.

Embodiment II-37. The engineered CasX protein of embodiment II-28, comprising at least two NLS, wherein the at least two NLS are positioned at or near the N-terminus and at or near the C-terminus of the protein.

Embodiment II-38. The engineered CasX protein of any one of embodiments II-21 to II-37, wherein the engineered CasX protein is capable of forming a ribonuclear protein complex (RNP) with an ERS.

Embodiment II-30. An engineered CasX protein, comprising a sequence of any of SEQ ID NOS: 24916-27856, wherein the mutations result in an improved characteristic compared to SEQ ID NO: 228.

Embodiment II-31. The engineered CasX protein of embodiment II-30 wherein the improved characteristic is one or more of improved ability to utilize a greater spectrum of PAM sequences in the editing of target nucleic acid, increased nuclease activity, improved editing efficiency, improved editing specificity for the target nucleic acid, decreased off-target editing, increased percentage of a eukaryotic genome that can be efficiently edited, improved ability to form cleavage-competent RNP with an ERS, and improved stability of an RNP complex.

Embodiment II-32. The engineered CasX protein of embodiment II-30, wherein the improved characteristic comprises increased nuclease activity at a target nucleic sequence comprising an TTC, ATC, GTC, or CTC PAM sequence relative to nuclease activity of the sequence of SEQ ID NO: 228.

Embodiment II-39. A gene editing pair comprising a ERS and an engineered CasX protein, the pair comprising:
a. a ERS of any one of embodiments II-1 to II-20; and
b. an engineered CasX protein of any one of embodiments II-21 to II-38.

Embodiment II-40. The gene editing pair of embodiment II-39, wherein the ERS and the engineered CasX protein are capable of forming a ribonuclear protein complex (RNP).

Embodiment II-41. The gene editing pair of embodiment II-39 or II-40, wherein the ERS and the engineered CasX protein are associated together as a ribonuclear protein complex (RNP).

Embodiment II-42. The gene editing pair of embodiment II-40 or II-41, wherein an RNP of the engineered CasX protein and the ERS exhibit at least one or more improved characteristics as compared to an RNP comprising the sequences of SEQ ID NO: 156 and SEQ ID NO: 228.

Embodiment II-43. The gene editing pair of embodiment II-42, wherein the improved characteristic is selected from one or more of the group consisting of increased binding affinity of the engineered CasX protein to the ERS; increased binding affinity to a target nucleic acid; increased ability to utilize a greater spectrum of one or more PAM sequences, including ATC, CTC, GTC, or TTC, in the editing of target nucleic acid; increased editing specificity of the target nucleic acid; increased nuclease activity; decreased off-target cleavage of the target nucleic acid; increased RNP stability; increased ability to form cleavage-competent RNP.

Embodiment II-44. The gene editing pair of any one of embodiments II-39 to II-43, wherein the RNP comprising the engineered CasX protein and the ERS exhibits greater editing efficiency and/or binding of a target nucleic acid sequence in the target nucleic acid when any one of the PAM sequences TTC, ATC, GTC, or CTC is located 1 nucleotide 5′ to the non-target strand of the protospacer having identity with the targeting sequence of the ERS in a cellular assay system compared to the editing efficiency and/or binding of an RNP comprising an earlier generation CasX protein and a reference ERS in a comparable assay system.

Embodiment II-45. A nucleic acid comprising a sequence that encodes the ERS of any one of embodiments II-1 to II-20.

Embodiment II-46. A nucleic acid comprising a sequence that encodes the engineered CasX protein of any one of embodiments II-21 to II-38.

Embodiment II-47. The nucleic acid of embodiment II-46, wherein the nucleic acid is mRNA.

Embodiment II-48. A vector comprising the ERS of any one of embodiments II-1 to II-20, the engineered CasX protein of any one of embodiments II-21 to II-38, or the nucleic acid of any one of embodiment II-45 or II-46.

Embodiment II-49. The vector of embodiment II-48, wherein the vector comprises a promoter operably linked to the nucleic acid.

Embodiment II-50. The vector of embodiment II-48 or II-49, wherein the vector is selected from the group consisting of a retroviral vector, a lentiviral vector, an adenoviral vector, an adeno-associated viral (AAV) vector, a herpes simplex virus (HSV) vector, a CasX delivery particle (XDP), a plasmid, a minicircle, a nanoplasmid, a DNA vector, and an RNA vector.

Embodiment II-51. The vector of embodiment II-50, wherein the vector is an AAV vector.

Embodiment II-52. The vector of embodiment II-51, wherein the AAV vector is selected from AAV1, AAV2, AAV3, AAV4, AAV5, AAV6, AAV7, AAV8, AAV9, AAV10, AAV 11, AAV12, AAV 9.45, AAV 9.61, AAV-Rh74, or AAVRh10.

Embodiment II-53. The vector of embodiment II-50, wherein the vector is a retroviral vector.

Embodiment II-54. The vector of embodiment II-50, wherein the vector is an XDP comprising one or more components of a gag polyprotein.

Embodiment II-55. The vector of embodiment II-54, wherein the engineered CasX protein and the ERS are associated together in an RNP.

Embodiment II-56. The vector of embodiment II-54 or II-55, comprising a glycoprotein tropism factor.

Embodiment II-57. The vector of embodiment II-56, wherein the glycoprotein tropism factor has binding affinity for a cell surface marker of a target cell and facilitates entry of the XDP into the target cell.

Embodiment II-58. A host cell comprising the vector of any one of embodiments II-48 to II-57.

Embodiment II-59. The host cell of embodiment II-58, wherein the host cell is selected from the group consisting of Baby Hamster Kidney fibroblast (BHK) cells, human embryonic kidney 293 (HEK293) cells, human embryonic kidney 293T (HEK293T) cells, NS0 cells, SP2/0 cells, YO myeloma cells, P3X63 mouse myeloma cells, PER cells, PER.C6 cells, hybridoma cells, NIH3T3 cells, CV-1 (simian) in Origin with SV40 genetic material (COS) cells, HeLa, Chinese hamster ovary (CHO) cells, or yeast cells.

Embodiment II-60. A lipid nanoparticle comprising the nucleic acid of any one of embodiments II-45 to II-47.

Embodiment II-61. A method of modifying a target nucleic acid in a cell, comprising contacting the target nucleic acid of the cell with: i) the gene editing pair of any one of embodiments II-39 to II-44; ii) one or more nucleic acids encoding the gene editing pair of (i); iii) a vector comprising the nucleic acid of (iii); iv) an XDP comprising the gene editing pair of (i); v) an LNP of embodiment II-60; or vi) combinations of two or more of (i) to (v), wherein the contacting of the target nucleic acid modifies the target nucleic acid.

Embodiment II-62. The method of embodiment II-61, comprising contacting the target with a plurality of gene editing pairs comprising a first and a second, or a plurality of ERS comprising targeting sequences complementary to different or overlapping regions of the target nucleic acid.

Embodiment II-63. The method of embodiment II-61, comprising contacting the target with a plurality of nucleic acids encoding gene editing pairs comprising a first and a second, or a plurality of ERS comprising targeting sequences complementary to different or overlapping regions of the target nucleic acid.

Embodiment II-64. The method of embodiment II-61, comprising contacting the target with a plurality of XDP comprising gene editing pairs comprising a first and a second, or a plurality of ERSs comprising targeting sequences complementary to different or overlapping regions of the target nucleic acid.

Embodiment II-65. The method of any one of embodiment II-60, wherein the contacting comprises binding the target nucleic acid with the gene editing pair and introducing one or more single-stranded breaks in the target nucleic acid, wherein the modifying comprises introducing a mutation, an insertion, or a deletion in the target nucleic acid.

Embodiment II-66. The method of any one of embodiments II-60 to II-64 wherein the contacting comprises binding the target nucleic acid and introducing one or more double-stranded breaks in the target nucleic acid, wherein the modifying comprises introducing a mutation, an insertion, or a deletion in the target nucleic acid.

Embodiment II-67. The method of any one of embodiments II-61 to II-66, wherein the modifying of the cell occurs in vitro.

Embodiment II-68. The method of any one of embodiments II-61 to II-64, wherein modifying of the cell occurs in vivo.

Embodiment II-69. The method of any one of embodiments II-60 to II-68, wherein the cell is a eukaryotic cell.

Embodiment II-70. The method of embodiment II-69, wherein the eukaryotic cell is selected from the group consisting of a rodent cell, a mouse cell, a rat cell, a primate cell, and a non-human primate cell.

Embodiment II-71. The method of embodiment II-69, wherein the eukaryotic cell is a human cell.

Embodiment II-72. The method of any one of embodiments II-60 to II-71, wherein the cell is selected from the group consisting of an embryonic stem cell, an induced pluripotent stem cell, a germ cell, a fibroblast, an oligodendrocyte, a glial cell, a hematopoietic stem cell, a neuron progenitor cell, a neuron, a muscle cell, a bone cell, a hepatocyte, a pancreatic cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, a parenchymal cell, an epithelial cell, an endothelial cell, a mesothelial cell, a fibroblast cell, an osteoblast cell, a chondrocyte cell, an exogenous cell, an endogenous cell, a stem cell, a hematopoietic stem cell, a bone-marrow derived progenitor cell, a myocardial cell, a skeletal cell, a fetal cell, an undifferentiated cell, a multi-potent progenitor cell, a unipotent progenitor cell, a monocyte, a cardiac myoblast, a skeletal myoblast, a macrophage, a capillary endothelial cell, a xenogenic cell, an allogenic cell, an autologous cell, and a post-natal stem cell.

Embodiment II-73. The method of any one of embodiments II-68 to II-72, wherein the cell is in a subject.

Embodiment II-74. The method of embodiment II-73, wherein the modifying occurs in the cells of the subject having a mutation in an allele of a gene wherein the mutation causes a disease or disorder in the subject.

Embodiment II-75. The method of embodiment II-74, wherein the modifying changes the mutation to a wild type allele of the gene or results in the expression of a functional gene product.

Embodiment II-76. The method of embodiment II-74, wherein the modifying knocks down or knocks out the allele of the gene causing the disease or disorder in the subject.

Embodiment II-77. A composition, comprising the engineered CasX protein of any one of embodiments II-21 to II-38.

Embodiment II-78. The composition of embodiment II-77, comprising the ERS of any one of embodiments II-13 to II-20.

Embodiment II-79. The composition of embodiment II-78, wherein the protein and the ERS are associated together in a ribonuclear protein complex (RNP).

Embodiment II-80. A composition, comprising a ERS of any one of embodiments II-1 to II-20.

Embodiment II-81. The composition of embodiment II-80, comprising the engineered CasX protein of any one of embodiments II-21 to II-38.

Embodiment II-82. The composition of embodiment II-81, wherein the engineered CasX protein and the ERS are associated together in a ribonuclear protein complex (RNP).

Embodiment II-83. An engineered CasX protein comprising any one of the sequences listed in Table 14.

Embodiment II-84. A ERS comprising any one of the ERS variant sequences listed in Table 15 or Table 26.

Embodiment II-85. The ERS of embodiment II-84, comprising a targeting sequence of at least 10 to 30 nucleotides complementary to a target nucleic acid.

Embodiment II-86. The ERS of embodiment II-85, wherein the targeting sequence has 20 nucleotides.

Embodiment II-87. The ERS of embodiment II-85, wherein the targeting sequence has 19 nucleotides.

Embodiment II-88. The ERS of embodiment II-85, wherein the targeting sequence has 18 nucleotides.

Embodiment II-89. The ERS of embodiment II-85, wherein the targeting sequence has 17 nucleotides.

Embodiment II-90. The ERS of embodiment II-85, wherein the targeting sequence has 16 nucleotides.

Embodiment II-91. The ERS of embodiment II-85, wherein the targeting sequence has 15 nucleotides.

Embodiment III-1. An engineered ribonucleic acid scaffold (ERS) comprising a sequence of SEQ ID NO: 17, or a sequence having at least about 70% sequence identity thereto, comprising an extended stem loop sequence of SEQ ID NO: 49739 and one or more mutations at positions selected from the group consisting of U11, U24, A29, and A87.

Embodiment III-2. The engineered ERS of embodiment III-1, comprising mutations at positions U11, U24, A29, and A87.

Embodiment III-3. The engineered ERS of embodiment III-1, comprising one or more mutations selected from the group consisting of U11C, U24C, A29C, and A87G.

Embodiment III-4. The engineered ERS of embodiment III-3, comprising mutations consisting of U11C, U24C, A29C, and A87G.

Embodiment III-5. An engineered ribonucleic acid scaffold (ERS) comprising a sequence of SEQ ID NO: 75, or a sequence having at least about 70% sequence identity thereto, modified to comprise an extended stem loop sequence of SEQ ID NO: 49739.

Embodiment III-6. The ERS of embodiment III-5, the sequence comprising regions selected from the group consisting of:
a. a 5′ end comprising a sequence of AC;
b. a pseudoknot stem I comprising a sequence of UGGCGCU;
c. a triplex loop comprising a sequence of SEQ ID NO: 49736;
d. a pseudoknot stem II comprising a sequence of AGCGCCA; and
e. a triplex region III comprising a sequence of CAGAG.

Embodiment III-7. An engineered ribonucleic acid scaffold (ERS), comprising the sequence of ACUGGCGCUUCUAUCUGAUUACUCUGAGCGCCAUCACCAGCGACUAUGUCGUAGU GGGUAAAGCUCCCUCUUCGGAGGGAGCAUCAGAG (SEQ ID NO: 156), or a sequence having at least about 96% sequence identity thereto.

Embodiment III-8. An engineered ribonucleic acid scaffold (ERS) comprising a sequence having at least about 70% sequence identity to

(i)
ACUGGCACUUCUAUCUGAUUACUCUGAGAGCCAUCACCAGCGACUAUGUCGUAUGGGUA
AAGCCGCUUACGGACUUCGGUCCGUAAGAGGCAUCAGAG (SEQ ID NO: 61);
or
(ii)
(SEQ ID NO: 156)
ACUGGCGCUUCUAUCUGAUUACUCUGAGCGCCAUCACCAGCGACUAUGUCGUAGUGGGU
AAAGCUCCCUCUUCGGAGGGAGCAUCAGAG;

comprising one or more modifications in the sequence, wherein the one or more modifications result in an improved characteristic compared to unmodified SEQ ID NO: 61 or SEQ ID NO: 156.

Embodiment III-9. The ERS of embodiment III-8, comprising at least two modifications in the sequence, wherein the modifications result in an improved characteristic compared to unmodified SEQ ID NO: 61 or SEQ ID NO: 156.

Embodiment III-10. The ERS of embodiment III-8 or III-9, wherein the modification comprises:
a. a substitution of 1 to 30 consecutive nucleotides in one or more regions of the scaffold;
b. a deletion of 1 to 10 consecutive nucleotides in one or more regions of the scaffold;
c. an insertion of 1 to 10 consecutive nucleotides in one or more regions of the scaffold;
d. a substitution of the scaffold stem loop with an RNA stem loop sequence from a heterologous RNA source;
e. a substitution of the extended scaffold stem loop with an RNA stem loop sequence from a heterologous RNA source; or
f. any combination of (a)-(d).

Embodiment III-11. The ERS of any one of embodiments III-8 to III-10, wherein the modifications comprise mutations in one or more regions selected from the group consisting of a 5′ end, a pseudoknot stem, a triplex loop, a scaffold stem loop, an extended stem loop, and a triplex region III.

Embodiment III-12. The ERS of any one of embodiments III-8 to III-10, wherein the modifications comprise mutations in at least two regions of the ERS, wherein the regions are selected from the group consisting of a 5′ end, a pseudoknot stem I, a triplex loop, a pseudoknot stem II, a scaffold stem loop, an extended stem loop, and a triplex region III.

Embodiment III-13. The ERS of any one of embodiments III-8 to III-12, wherein the mutations are selected from the group consisting of the mutations of Tables 44, 45, and 47.

Embodiment III-14. The ERS of embodiment III-13, wherein sequences of the individual mutated regions have the sequences of:
a. SEQ ID NOS: 739-753 in the 5′ end region;
b. SEQ ID NOS: 754-772 in the triplex loop region;
c. SEQ ID NOS: 773-791 in the triplex region;
d. SEQ ID NOS: 792-841 in the pseudoknot region;
e. SEQ ID NOS: 842-869 in the scaffold stem region; and/or
f. SEQ ID NOS: 870-907 in the extended stem region.

Embodiment III-15. The ERS of embodiment III-13, wherein the ERS comprises paired combinations of individual mutated sequences from different or the same regions.

Embodiment III-16. The ERS of embodiment III-15, wherein the ERS comprises a sequence selected from the group consisting of SEQ ID NOS: 11,568-22,227 and 23,572-24,915, or a sequence having at least 70% sequence identity thereto.

Embodiment III-17. The ERS of embodiment III-15, wherein the ERS comprises a sequence selected from the group consisting of SEQ ID NOS: 11,568-22,227 and 23,572-24,915.

Embodiment III-18. The ERS of any one of embodiments III-7 to III-17, wherein the scaffold has 85-100 nucleotides, or any integer in between.

Embodiment III-19. An ERS comprising a sequence selected from the group consisting of SEQ ID NOS: 156, 739-907, 739-907, 11568-22227, 23572-24915, and 49719-49735, or a sequence having at least about 90%, at least about 95%, at least about 98%, or at least about 99% sequence identity thereto, wherein the ERS comprises an improved characteristic compared to the sequence of SEQ ID NO: 17.

Embodiment III-20. The ERS of embodiment III-19, wherein the ERS comprises a sequence selected from the group consisting of SEQ ID NOS: 156, 739-907, 11568-22227, 23572-24915, and 49719-49735, wherein the ERS comprises an improved characteristic compared to the sequence of SEQ ID NO: 17, when assayed in an in vitro cell-based assay under comparable conditions.

Embodiment III-21. The ERS of embodiment III-19 or III-20, wherein the improved characteristic is one or more functional properties selected from the group consisting of improved binding to a CasX nuclease to form a ribonucleoprotein (RNP), improved folding stability of the ERS, increased half-life in a cell, increased transcriptional efficiency, enhanced ability to synthetically manufacture the ERS, improved editing activity of a target nucleic acid by an RNP comprising the ERS, and improved editing specificity by an RNP comprising the ERS.

Embodiment III-22. The ERS of any one of embodiments III-1 to III-21, wherein the ERS comprises one or more heterologous RNA sequences in the extended stem loop.

Embodiment III-23. The ERS of embodiment III-22, wherein the heterologous RNA is selected from the group consisting of a MS2 hairpin, Qβ hairpin, U1 hairpin II, Uvsx hairpin, and a PP7 stem loop, or sequence variants thereof.

Embodiment III-24. The ERS of embodiment III-22 or III-23, wherein the heterologous RNA is capable of binding a protein, a RNA, a DNA, or a small molecule.

Embodiment III-25. The ERS of any one of embodiments III-1 to III-24, wherein the ERS comprises a Rev response element (RRE), or a portion thereof.

Embodiment III-26. The ERS of any one of embodiments III-1 to III-25, comprising a targeting sequence linked at the 3′ end of the ERS that is complementary to a target nucleic acid sequence.

Embodiment III-27. The ERS of embodiment III-26, wherein the targeting sequence has 15-20 nucleotides.

Embodiment III-28. The ERS of embodiment III-27, wherein the targeting sequence has 20 nucleotides.

Embodiment III-29. The ERS of any one of embodiments III-26 to III-28, wherein the ERS and linked targeting sequence has 100-115 nucleotides.

Embodiment III-30. The ERS of any one of embodiments III-1 to III-29, wherein the CpG content of the ERS is reduced or depleted.

Embodiment III-31. The ERS of embodiment III-30, wherein the CpG content is less than about 10%, less than about 5%, or less than about 1%.

Embodiment III-32. The ERS of any one of embodiments III-1 to III-31, wherein the ERS comprises one or more chemical modifications to the sequence.

Embodiment III-33. The ERS of embodiment III-32, wherein the chemical modification is addition of a 2′O-methyl group to one or more nucleotides of the sequence.

Embodiment III-34. The ERS of embodiment III-32 or III-33, wherein one or more nucleotides on either or both of the 5′ and 3′ terminal ends of the ERS are modified by an addition of a 2′O-methyl group.

Embodiment III-35. The ERS of any one of embodiments III-32 to III-34, wherein the chemical modification is substitution of a phosphorothioate bond between two or more nucleosides of the sequence.

Embodiment III-36. The ERS of any one of embodiments III-32 to III-35, wherein the chemical modification is a substitution of phosphorothioate bonds between two or more nucleotides on either or both of the 5′ and 3′ terminal ends of the ERS.

Embodiment III-37. The ERS of any one of embodiments III-32 to III-36, wherein the chemically modified ERS comprises a sequence selected from the group consisting of SEQ ID NOS: 49750-49758, 49760-49768, and 49770-49749.

Embodiment III-38. The ERS of any one of embodiments III-32 to III-37, wherein the chemically modified ERS comprises a sequence of SEQ ID NO: 49770.

Embodiment III-39. The ERS of embodiment III-37 or III-38, wherein the chemically modified ERS sequence is modified with a 20 nucleotide targeting sequence complementary to a target nucleic acid.

Embodiment III-40. The ERS of any one of embodiments III-32 to III-39, wherein the chemical modifications result in reduced susceptibility of the ERS to degradation by cellular RNase compared to an unmodified ERS.

Embodiment III-41. The ERS of any one of embodiments III-1 to III-40, wherein the ERS is capable of forming a ribonucleoprotein (RNP) complex with a CasX protein.

Embodiment III-42. An engineered CasX protein, comprising a sequence having at least two mutations in the sequence of CasX 515 (SEQ ID NO: 49699) wherein the mutations result in an improved characteristic compared to unmodified CasX 515.

Embodiment III-43. The engineered CasX protein of embodiment III—, wherein the improved condition is determined in an in vitro assay under comparable conditions.

Embodiment III-44. The engineered CasX protein of embodiment III-42, wherein the mutations are selected from the group consisting of:
a. an amino acid substitution;
b. an amino acid deletion;
c. an amino acid insertion; and
d. any combination of (a)-(c).

Embodiment III-45. The engineered CasX protein of any one of embodiments III-42, wherein engineered CasX protein comprises:
a. an oligonucleotide binding domain (OBD)-I comprising an amino acid sequence comprising one or more mutations relative to the sequence of SEQ ID NO: 295;
b. a helical I-I domain comprising an amino acid sequence comprising one or more mutations relative to the sequence of SEQ ID NO: 296;
c. an NTSB domain comprising an amino acid sequence comprising one or more mutations relative to the sequence of SEQ ID NO: 297;
d. a helical I-II domain comprising an amino acid sequence comprising one or more mutations relative to the sequence of SEQ ID NO: 298;
e. a helical II domain comprising an amino acid sequence comprising one or more mutations relative to the sequence of SEQ ID NO: 299;
f. a RuvC-I domain comprising an amino acid sequence comprising one or more mutations relative to the sequence of SEQ ID NO: 301;
g. a target strand loading (TSL) domain comprising an amino acid sequence comprising one or more mutations relative to the sequence of SEQ ID NO: 302; or
h. any combination of (a)-(g).

Embodiment III-46. The engineered CasX protein of embodiment III-45, wherein:
a. the OBD-I comprises one or more mutations relative to the sequence of SEQ ID NO: 295 selected from the group consisting of an I3G substitution, an insertion of a G at position 4, a K4G substitution, an insertion of a G at position 5, a K8G substitution, an insertion of an R at position 26, and a R34P substitution;
b. the helical I-I domain comprises an R7Q substitution relative to the amino acid sequence of SEQ ID NO: 296;
c. the NTSB domain comprises one or more mutations relative to the sequence of SEQ ID NO: 297 selected from the group consisting of an L68K substitution, an L68Q substitution, an A70Y substitution, an A70D substitution, and an A70S substitution;
d. the helical I-II domain comprises one or more mutations relative to the sequence of SEQ ID NO: 298 selected from the group consisting of a G32T substitution, an M112T substitution, and an M112W substitution;
e. the helical II domain comprises one or more mutations relative to the sequence of SEQ ID NO: 299 selected from the group consisting of a Y65T substitution and an E148D substitution;
f. the RuvC-I domain comprises an S5IR substitution relative to the sequence of SEQ ID NO: 301;
g. the TSL domain comprises one or more mutations relative to the sequence of SEQ ID NO: 302 selected from the group consisting of a V15M substitution, a T76D substitution, and an S80Q substitution; or
h. any combination of (a)-(g).

Embodiment III-47. The engineered CasX protein of embodiment III-45 or III-46, wherein:
a. the OBD-I comprises a sequence selected from the group consisting of SEQ ID NOS: 295, 49800, 49803-49808, and 49822-49833, or a sequence having at least about 90%, at least about 95%, at least about 98%, at least about 99% sequence identity thereto;
b. the helical I-I domain comprises a sequence selected from the group consisting of SEQ ID NOS: 296 and 49809, or a sequence having at least about 90%, at least about 95%, at least about 98%, or at least about 99% sequence identity thereto;
c. the NTSB domain comprises a sequence selected from the group consisting of SEQ ID NOS: 297, 49802, 49810, 49811, 49812, 49818, and 49835-49840, or a sequence having at least about 90%, at least about 95%, at least about 98%, or at least about 99% sequence identity thereto;
d. the helical I-II domain comprises a sequence selected from the group consisting of SEQ ID NOS: 298, 49801, 49813-49814, and 49842, or a sequence having at least about 90%, at least about 95%, at least about 98%, or at least about 99% sequence identity thereto;
e. the helical II domain comprises a sequence selected from the group consisting of SEQ ID NOS: 299, 49815-49816, and 49843, or a sequence having at least about 90%, at least about 95%, at least about 98%, or at least about 99% sequence identity thereto;
f. the RuvC-I domain comprises a sequence selected from the group consisting of SEQ ID NOS: 301 and 49821, or a sequence having at least about 90%, at least about 95%, at least about 98%, or at least about 99% sequence identity thereto;
g. the TSL domain comprises a sequence selected from the group consisting of SEQ ID NOS: 302, 49817, 49819, 49820, and 49844-49846, or a sequence having at least about 90%, at least about 95%, at least about 98%, or at least about 99% sequence identity thereto; or
h. any combination of (a)-(g).

Embodiment III-48. The engineered CasX protein of any one of embodiments III-45 to III-47, wherein the engineered CasX protein further comprises:
a. an OBD-II comprising the sequence of SEQ ID NO: 300, or a sequence having at least about 90%, at least about 95%, at least about 98%, or at least about 99% sequence identity thereto; and/or
b. a RuvC-II domain comprising the sequence of SEQ ID NO: 303, or a sequence having at least about 90%, at least about 95%, at least about 98%, or at least about 99% sequence identity thereto.

Embodiment III-49. The engineered CasX protein of any one of embodiments III-42 to III-48, wherein the engineered CasX protein comprises, from N- to C-terminus, an OBD-I domain, a helical I-I domain, an NTSB domain, a helical I-II domain, a helical II domain, an OBD-II, a RuvC-I domain, a TSL domain, and a RuvC-II domain, with each domain comprising a sequence as set forth in Table 21.

Embodiment III-50. The engineered CasX protein of any one of embodiments III-42 to III-49, wherein the two mutations are selected from the group consisting of the paired mutations as set forth in Table 22.

Embodiment III-51. The engineered CasX protein of any one of embodiments III-42 to III-49, wherein the two mutations are selected from the group consisting of the following pairs: 4.I.G & 64.R.Q, 4.I.G & 169.L.K, 4.I.G & 169.L.Q, 4.I.G & 171.A.D, 4.I.G & 171.A.Y, 4.I.G & 171.A.S, 4.I.G & 224.G.T, 41G & 304.M.T, 4.I.G & 398.Y.T, 4.I.G & 826.V.M, 4.I.G & 887.T.D, 4.I.G & 891.S.Q, 5.-.G & 64.R.Q, 5.-.G & 169.L.K, 5.-.G & 169.L.Q, 5.-.G & 171.A.D, 5.-.G & 171.A.Y, 5.-.G & 171.A.S, 5.-.G & 224.G.T, 5.-.G & 304.M.T, 5.-.G & 398.Y.T, 5.-.G & 826.V.M, 5.-.G & 887.T.D, 5.-.G & 891.S.Q, 9.K.G & 64.R.Q, 9.K.G & 169.L.K, 9.K.G & 169.L.Q, 9.K.G & 171.A.D, 9.K.G & 171.A.Y, 9.K.G & 171.A.S, 9.K.G & 224.G.T, 9.K.G & 304.M.T, 9.K.G & 398.Y.T, 9.K.G & 826.V.M, 9.K.G & 887.T.D, 9.K.G & 891.S.Q, 27.-.R & 64.R.Q, 27.-.R & 169.L.K, 27.-.R & 169.L.Q, 27.-.R & 171.A.D, 27.-.R & 171.A.Y, 27.-.R & 171.A.S, 27.-.R & 224.G.T, 27.-.R & 304.M.T, 27.-.R & 398.Y.T, 27.-.R & 826.V.M, 27.-.R & 887.TD, 27.-.R & 891.S.Q, 35.R.P & 64.R.Q, 35.R.P & 169.L.K, 35.R.P & 169.L.Q, 35.R.P & 171.A.D, 35.R.P & 171.AY, 35.R.P & 171.A.S, 35.R.P & 224.G.T, 35.R.P & 304.M.T, 35.R.P & 398.Y.T, 35.R.P & 826.V.M, 35.R.P & 887.T.D, 35.R.P & 891.S.Q, 887.T.D & 891.S.Q, 64.R.Q & 169.L.K, 64.R.Q & 169.L.Q, 64.R.Q & 171.AD, 64.R.Q & 171.A.Y, 64.R.Q & 171.A.S, 64.R.Q & 224.G.T, 64.R.Q & 304.M.T, 64.R.Q & 398.Y.T, 64.R.Q & 826.V.M, 64.R.Q & 887.T.D, 64.R.Q & 891.S.Q, 169.L.K & 171.A.D, 169.L.K & 171.A.Y, 169.L.K & 171.A.S, 169.L.K & 224.G.T, 169.L.K & 304.M.T, 169.L.K & 398.Y.T, 169.L.K & 826.V.M, 169.L.K & 887.T.D, 169.L.K & 891.S.Q, 169.L.Q & 171.A.D, 169.L.Q & 171.A.Y, 169.L.Q & 171.A.S, 169.L.Q & 224.G.T, 169.L.Q & 304.M.T, 169.L.Q & 398.Y.T, 169.L.Q & 826.V.M, 169.L.Q & 887.T.D, 169.L.Q & 891.S.Q, 171.A.D & 224.G.T, 171.A.D & 304.M.T, 171.A.D & 398.Y.T, 171.A.D & 826.V.M, 171.A.D & 887.T.D, 171.A.D & 891.S.Q, 171.A.Y & 224.G.T, 171.A.Y & 304.M.T, 171.A.Y & 398.Y.T, 171.A.Y & 826.V.M, 171.A.Y & 887.T.D, 171.A.Y & 891.S.Q, 171.A.S & 224.G.T, 171.A.S & 304.M.T, 171.A.S & 398.Y.T, 171.A.S & 826.V.M, 171.A.S & 887.T.D, 171.A.S & 891.S.Q, 4.I.G & 35.R.P, 224.G.T & 304.M.T, 224.G.T & 398.Y.T, 224.G.T & 826.V.M, 224.G.T & 887.T.D, 224.G.T & 891.S.Q, 5.-.G & 35.R.P, 4.I.G & 27.-.R, 304.M.T & 398.Y.T, 304.M.T & 826.V.M, 304.M.T & 887.T.D, 304.M.T & 891.S.Q, 9.K.G & 35.R.P, 5.-.G & 27.-.R, 4.I.G & 9.K.G, 398.Y.T & 826.V.M, 398.Y.T & 887.T.D, 398.Y.T & 891.S.Q, 27.-.R & 35.R.P, 9.K.G & 27.-.R, 5.-.G & 9.K.G, 4.I.G & 5.-.G, 826.V.M & 887.T.D, 826.V.M & 891.S.Q, 5.K.G & 27.-.R, 5.K.G & 169.L.K, 5.K.G & 171.A.D, 5.K.G & 304.M.T, 5.K.G & 398.Y.T, 5.K.G & 891.S.Q, 6.-.G & 27.-.R, 6.-.G & 169.L.K, 6.-.G & 171.AD, 6.-.G & 304.M.T, 6.-.G & 398.Y.T, 6.-.G & 891. S.Q, 304.M.W & 27.-.R, 304.M.W & 169.L.K, 304.M.W & 171.A.D, 304.M.W & 398.Y.T, 304.M.W & 891.S.Q, 481.E.D & 27.-.R, 481.E.D & 169.L.K, 481.E.D & 171.AD, 481.E.D & 304.M.T, 481.E.D & 398.Y.T, 481.E.D & 891.S.Q, 698.S.R & 27.-.R, 698.S.R & 169.L.K, 698.S.R & 171.A.D, 698.S.R & 304.M.T, 698.S.R & 398.Y.T, and 698.S.R & 891.S.Q. The engineered CasX protein of embodiment III-42, wherein comprising three mutations selected from the group consisting of (a) 27.-.R, 169.L.K, and 329.G.K; (b) 27.-.R, 171.A.D, and 224.G.T; and (c) 35.R.P, 171.A.Y, and 304.M.T, wherein the mutations result in an improved characteristic compared to unmodified CasX 515.

Embodiment III-52. The engineered CasX protein of any one of embodiments III-42 to III-51, comprising a sequence selected from SEQ ID NOS: 24916-49628, 49746-49747, and 49871-49873, or a sequence having at least 70% sequence identity thereto.

Embodiment III-53. The engineered CasX protein of any one of embodiments III-42 to III-51, comprising a sequence selected from SEQ ID NOS: 24916-49628, 49746-49747, and 49871-49873.

Embodiment III-54. The engineered CasX protein of any one of embodiments III-42 to III-49, comprising a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 2, 7958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747 and 49871-49873, or a sequence having at least about 70%, at least about 80%, at least about 90%, at least about 95%, at least about 98%, or at least about 99% sequence identity thereto.

Embodiment III-55. The engineered CasX protein of any one of embodiments III-42 to III-50, comprising a sequence selected from the group consisting of SEQ 1D NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 284149, 28456, 28464, 28465, 28470, 28477, 28481, 28490, 284198, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873.

Embodiment III-56. The engineered CasX protein of any one of embodiments III-42 to III-55, wherein the improved characteristic is one or more of editing activity, improved editing specificity, improved specificity ratio, improved editing activity and editing specificity, or improved editing activity and improved specificity ratio.

Embodiment III-57. The engineered CasX protein of any one of embodiments III-42 to III-55, wherein the engineered CasX comprises a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 or a sequence having at least about 70%, at least about 80%, at least about 90%, at least about 95%, at least about 98%, or at least about 99% sequence identity thereto, wherein the engineered CasX exhibits improved editing activity compared to unmodified CasX 515.

Embodiment III-58. The engineered CasX protein of any one of embodiments III-42 to III-55, wherein the engineered CasX comprises a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873, or a sequence having at least about 70%, at least about 80%, at least about 90%, at least about 95%, at least about 98%, or at least about 99% sequence identity thereto, wherein the engineered CasX exhibits improved editing specificity compared to unmodified CasX 515

Embodiment III-59. The engineered CasX protein of any one of embodiments III-42 to III-55, wherein the engineered CasX comprises a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 or a sequence having at least about 70%, at least about 80%, at least about 90%, at least about 95%, at least about 98%, or at least about 99% sequence identity thereto, wherein the engineered CasX exhibits improved editing activity and specificity compared to unmodified CasX 515.

Embodiment III-60. The engineered CasX protein of any one of embodiments II-42 to III-55, wherein the engineered CasX comprises a sequence selected from the group consisting of SEQ ID NOS: 27865, 27952, 27954, 27955, 27958, 27959, 27973, 28009, 28018, 28048, 28101, 28123, 28137, 28285, 28296, 28301, 28305, 28314, 28323, 28368, 28369, 28370, 28378, 28387, 28438, 28447, 28477, 28481, 28498, 28515, 28524, 28532, 28661, 28799, 28925, 29022, 29266, 29308, 29371, 29560, 29749, 29917, 30888, 31244, 33212, 33512, 34088, 34870, 35422, 35507, 43373, 49872, and 49873, or a sequence having at least about 70%, at least about 80%, at least about 90%, at least about 95%, at least about 98%, or at least about 99% sequence identity thereto, wherein the engineered CasX exhibits improved specificity ratio compared to unmodified CasX 515.

Embodiment III-61. The engineered CasX protein of any one of embodiments III-42 to III-55, wherein the engineered CasX comprises a sequence selected from the group consisting of SEQ ID NOS: 27952, 27958, 28101, 28123, 28137, 28285, 28368, 28370, 28378, 28387, 28438, 28799, 28925, 29022, 29308, 29749, 29917, 30888, 34870, 43373, and 49873, or a sequence having at least about 70%, at least about 80%, at least about 90%, at least about 95%, at least about 98%, or at least about 99% sequence identity thereto, wherein the engineered CasX exhibits improved editing activity and improved editing specificity compared to an unmodified CasX 515.

Embodiment III-62. The engineered CasX protein of any one of embodiments III-42 to III-61, wherein the improved characteristic is at least about 0.1-fold to about 10-fold improved in the in vitro assay.

Embodiment III-63. The engineered CasX variant of any one of embodiments III-1 to 111-55, wherein the engineered CasX protein is a catalytically inactive CasX (dCasX) protein.

Embodiment III-64. The engineered CasX variant of embodiment III-63, wherein the dCasX comprises a mutation at residues:
a. D672A, and/or E769A, and/or D935A corresponding to the CasX protein of SEQ ID NO:1; or D659A, and/or E756A, and/or D922A corresponding to the CasX protein of SEQ ID NO: 2.

Embodiment III-65. An engineered CasX protein comprising:
a. an NTSB domain sequence of SEQ ID NO: 297, or a sequence having at least about 90%, or at least about 95% sequence identity thereto;
b. a RuvC-II domain sequence of SEQ ID NO: 303, or a sequence having at least about 90%, or at least about 95% sequence identity thereto; and;
c. a helical I-II domain sequence of SEQ ID NO: 298, or a sequence having at least about 90%, or at least about 95% sequence identity thereto, comprising an amino acid substitution of position G137 relative to the sequence of SEQ ID NO: 298, wherein the substituted position G137 relative to the sequence of SEQ ID NO: 298 comprises a hydrophilic amino acid residue.

Embodiment III-66. The engineered CasX protein of embodiment III-65, wherein the hydrophilic amino acid residue is lysine or asparagine.

Embodiment III-67. The engineered CasX protein of embodiment III-65 or 111-66, comprising:
a. an OBD-I domain sequence of SEQ ID NO: 295, or a sequence having at least about 90%, or at least about 95% sequence identity thereto;
b. a helical I-I domain sequence of SEQ ID NO: 296, or a sequence having at least about 90%, or at least about 95% sequence identity thereto;
c. an OBD-II domain sequence of SEQ ID NO: 300, or a sequence having at least about 90%, or at least about 95% sequence identity thereto;
d. a RuvC-I domain sequence of SEQ ID NO: 301, or a sequence having at least about 90%, or at least about 95% sequence identity thereto; and
e. a TSL domain sequence of SEQ ID NO: 302, or a sequence having at least about 90%, or at least about 95% sequence identity thereto.

Embodiment III-68. The engineered CasX protein of any one of embodiments III-65 to 111-67, comprising a sequence of SEQ ID NO: 266, or a sequence having at least about 70%, at least about 80%, at least about 90%, at least about 95%, at least about 98%, at least about 99% sequence identity thereto, wherein the engineered CasX has an improved characteristic of the compared to the CasX of SEQ ID NO: 228.

Embodiment III-69. The engineered CasX protein of embodiment III-68, wherein the improved characteristic is one or more of improved ability to utilize a greater spectrum of protospacer adjacent motif (PAM) sequences in the editing of target nucleic acid, increased nuclease activity, increased editing of target nucleic acid, improved editing specificity for the target nucleic acid, decreased off-target editing, increased percentage of a eukaryotic genome that can be efficiently edited, improved ability to form cleavage-competent RNP with an ERS, and improved stability of an RNP complex.

Embodiment III-70. The engineered CasX protein of embodiment III-69, wherein the improved characteristic comprises increased editing specificity of target nucleic acid relative to the editing of the sequence of SEQ ID NO: 228, wherein the increase is at least about 1.01-fold, at least about 1.5-fold, at least about 2-fold, at least about 4-fold, at least about 10-fold, at least about 20-fold, at least about 30-fold, or at least about 40-fold greater.

Embodiment III-71. The engineered CasX protein of embodiment III-69, wherein the improved characteristic comprises decreased off-target editing relative to the off-target editing of the sequence of SEQ ID NO: 228.

Embodiment III-72. The engineered CasX protein of embodiment III-71, wherein the off-target editing is less than about 5%, less than about 4%, less than 3%, less than about 2%, less than about 1%, less than about 0.5%, less than 0.1%, when measured in silico, in an in vitro cell-free assay, or in a cell-based assay.

Embodiment III-73. The engineered CasX protein of any one of embodiments III-42 to III-72, comprising one or more nuclear localization signals (NLS), and, optionally, wherein the one or more NLS are linked to the engineered CasX protein or to an adjacent NLS with a linker peptide.

Embodiment III-74. The engineered CasX protein of embodiment III-73, wherein the NLS is selected from the group consisting of the sequences of SEQ ID NOS: 364-457 as set forth in Table 8.

Embodiment III-75. The engineered CasX protein of embodiment III-73 and III-74, wherein the linker peptide is selected from the group consisting of SR, RS, and SEQ ID NOS: 468-486.

Embodiment III-76. The engineered CasX protein of any one of embodiments III-73 to III-75, wherein the one or more NLS are positioned at or near the C-terminus of the protein.

Embodiment III-77. The engineered CasX protein of any one of embodiments III-73 to III-75, wherein the one or more NLS are positioned at or near at the N-terminus of the protein.

Embodiment III-78. The engineered CasX protein of any one of embodiments III-73 to III-75, comprising at least two NLS, wherein the at least two NLS are positioned at or near the N-terminus and at or near the C-terminus of the protein.

Embodiment III-79. The engineered CasX protein of any one of embodiments III-42 to III-78, wherein the engineered CasX protein is capable of forming a ribonuclear protein complex (RNP) with an ERS.

Embodiment III-80. A gene editing pair comprising a ERS and an engineered CasX protein, the pair comprising an ERS of any one of embodiments III-1 to III-41 and an engineered CasX protein of any one of embodiments III-42 to III-79.

Embodiment III-81. The gene editing pair of embodiment III-80, wherein the ERS and the engineered CasX protein are capable of forming a ribonuclear protein complex (RNP).

Embodiment III-82. The gene editing pair of embodiment III-80, wherein the ERS and the engineered CasX protein are associated together as a ribonuclear protein complex (RNP).

Embodiment III-83. The gene editing pair of any one of embodiments III-80 to III-82, wherein an RNP of the engineered CasX protein and the ERS exhibit at least one or more improved characteristics as compared to an RNP comprising the sequences of SEQ ID NO: 156 and SEQ ID NO: 228.

Embodiment III-84. The gene editing pair of embodiment III-83, wherein the improved characteristic is selected from one or more of the group consisting of increased binding affinity of the engineered CasX protein to the ERS, increased binding affinity to a target nucleic acid, increased ability to utilize a greater spectrum of one or more PAM sequences, including ATC, CTC, GTC, or TTC, in the editing of target nucleic acid, increased editing specificity of the target nucleic acid, increased nuclease activity, increased cleavage rate of the target nucleic acid, decreased off-target cleavage of the target nucleic acid, increased RNP stability, and increased ability to form cleavage-competent RNP.

Embodiment III-85. A nucleic acid comprising a sequence that encodes the ERS of any one of embodiments III-1 to III-41.

Embodiment III-86. The nucleic acid of embodiment III-85, wherein the sequence is depleted or devoid of CpG motifs.

Embodiment III-87. The nucleic acid of embodiment III-86, comprising a sequence selected from the group consisting of SEQ ID NOS: 535-556.

Embodiment III-88. A nucleic acid comprising a sequence that encodes the engineered CasX protein of any one of embodiments III-42 to III-79.

Embodiment III-89. The nucleic acid of embodiment III-86, wherein the sequence that encodes the engineered CasX protein is codon-optimized.

Embodiment III-90. The nucleic acid of embodiment III-89, wherein the sequence that encodes the engineered CasX protein is codon-optimized for expression in a human cell.

Embodiment III-91. The nucleic acid of embodiment III-88, wherein the sequence that encodes the engineered CasX protein is devoid or depleted of CpG motifs.

Embodiment III-92. The nucleic acid of embodiment III-91, comprising a sequence selected from the group consisting of SEQ ID NOS: 49850-49861.

Embodiment III-93. The nucleic acid of any one of embodiments III-88 to III-90, wherein the nucleic acid is messenger RNA (mRNA).

Embodiment III-94. A vector comprising:
a. the ERS of any one of embodiments II-1 to III-41;
b. the engineered CasX protein of any one of embodiments III-42 to III-79;
c. the nucleic acid of embodiment III-85 to III-87;
d. the nucleic acid of any one of embodiments III-88 to III-93; or
e. any combination of (a)-(d).

Embodiment III-95. The vector of embodiment III-94, wherein the vector comprises a promoter operably linked to the nucleic acid.

Embodiment III-96. The vector of embodiment III-94 or III-95, wherein the vector is selected from the group consisting of a retroviral vector, a lentiviral vector, an adenoviral vector, an adeno-associated viral (AAV) vector, a herpes simplex virus (HSV) vector, a CasX delivery particle (XDP), a plasmid, a minicircle, a nanoplasmid, a DNA vector, and an RNA vector.

Embodiment III-97. The vector of embodiment III-96, wherein the vector is an AAV vector.

Embodiment III-98. The vector of embodiment III-97, wherein the AAV vector is a serotype selected from AAV1, AAV2, AAV3, AAV4, AAV5, AAV6, AAV7, AAV8, AAV9, AAV10, AAV11, AAV12, AAV 9.45, AAV 9.61, AAV-Rh74, or AAVRh10.

Embodiment III-99. The vector of embodiment III-98, wherein the AAV vector comprises a transgene with inverted terminal repeat (ITR) sequences derived from AAV2.

Embodiment III-100. The vector of embodiment III-96, wherein the vector is a retroviral vector.

Embodiment III-101. The vector of embodiment III-96, wherein the vector is an XDP comprising one or more components of a gag polyprotein.

Embodiment III-102. The vector of embodiment III-101, wherein the XDP comprises the engineered CasX protein and the ERS associated together in an RNP.

Embodiment III-103. The vector of embodiment III-101 or 111-102, comprising a glycoprotein tropism factor.

Embodiment III-104. The vector of embodiment III-103, wherein the glycoprotein tropism factor has binding affinity for a cell surface marker of a target cell and facilitates entry of the XDP into the target cell.

Embodiment III-105. A host cell comprising the vector of any one of embodiments III-94 to III-104.

Embodiment III-106. The host cell of embodiment III-105, wherein the host cell is selected from the group consisting of a Baby Hamster Kidney fibroblast (BHK) cell, a human embryonic kidney 293 (HEK293) cell, a human embryonic kidney 293T (HEK293T) cell, a NS0 cell, a SP2/0 cell, a YO myeloma cell, a P3X63 mouse myeloma cell, a PER cell, a PER.C6 cell, a hybridoma cell, a NIH3T3 cell, a CV-1 (simian) in Origin with SV40 genetic material (COS) cell, a HeLa cell, a Chinese hamster ovary (CHO) cell, or a yeast cell.

Embodiment III-107. A lipid nanoparticle (LNP) comprising:
a. the ERS of any one of embodiments III-1 to III-41;
b. the nucleic acid of any one of embodiments III-85 to III-93; or
c. a combination of (a) and (b).

Embodiment III-108. The LNP of embodiment III-107, wherein the LNP comprises one or more components selected from the group consisting of an ionizable lipid, a helper phospholipid, a polyethylene glycol (PEG)-modified lipid, and cholesterol or a derivative thereof.

Embodiment III-109. The LNP of embodiment III-107, wherein the LNP comprises an ionizable lipid, a helper phospholipid, a polyethylene glycol (PEG)-modified lipid, and cholesterol or a derivative thereof.

Embodiment III-110. The LNP of any one of embodiments III-107 to III-109, wherein the LNP comprises a cationic lipid comprising a pKa of about 5 to about 8.

Embodiment III-111. A method of modifying a target nucleic acid in a cell, comprising introducing into the cell:
a. the gene editing pair of any one of embodiments III-80 to III-84;
b. one or more nucleic acids encoding the gene editing pair of (a);
c. a vector comprising the nucleic acid of (b);
d. an XDP comprising the gene editing pair of (a);
e. the LNP of any one of embodiments III-107 to III-110; or
f. combinations of two or more of (a) to (e),
   wherein the target nucleic acid of the cell targeted by the ERS is modified by the engineered CasX.

Embodiment III-112. The method of embodiment III-111, comprising contacting the target with a plurality of gene editing pairs comprising a first and a second, or three or four ERS comprising targeting sequences complementary to different or overlapping regions of the target nucleic acid.

Embodiment III-113. The method of embodiment III-111, comprising contacting the target with a plurality of nucleic acids encoding gene editing pairs comprising a first and a second, three, or four ERS comprising targeting sequences complementary to different or overlapping regions of the target nucleic acid.

Embodiment III-114. The method of embodiment III-111, comprising contacting the target with a plurality of XDP comprising gene editing pairs comprising a first and a second, or three, or four ERSs comprising targeting sequences complementary to different or overlapping regions of the target nucleic acid.

Embodiment III-115. The method of embodiment III-111, comprising contacting the target nucleic acid with the gene editing pair and introducing one or more single-stranded breaks in the target nucleic acid, wherein the modifying comprises introducing a mutation, an insertion, or a deletion in the target nucleic acid.

Embodiment III-116. The method of any one of embodiments III-112 to III-115, wherein the contacting comprises binding the target nucleic acid and introducing one or more double-stranded breaks in the target nucleic acid, wherein the modifying comprises introducing a mutation, an insertion, or a deletion in the target nucleic acid.

Embodiment III-117. The method of any one of embodiments III-111 to III-116, wherein the modifying corrects a mutation in the gene to wild-type or results in the ability of the cell to express a functional gene product.

Embodiment III-118. The method of any one of embodiments III-111 to III-116, wherein the modifying knocks down or knocks out the gene.

Embodiment III-119. The method of any one of embodiments III-111 to III-116, wherein the modifying of the cell occurs in vitro or ex vivo.

Embodiment III-120. The method of any one of embodiments III-111 to III-114, wherein modifying of the cell occurs in vivo.

Embodiment III-121. The method of any one of embodiments III-111 to III-120, wherein the cell is a eukaryotic cell.

Embodiment III-122. The method of embodiment III-121, wherein the eukaryotic cell is selected from the group consisting of a rodent cell, a mouse cell, a rat cell, a primate cell, and a non-human primate cell.

Embodiment III-123. The method of embodiment III-121, wherein the eukaryotic cell is a human cell.

Embodiment III-124. The method of any one of embodiments III-111 to III-123, wherein the cell is selected from the group consisting of an embryonic stem cell, an induced pluripotent stem cell, a germ cell, a fibroblast, an oligodendrocyte, a glial cell, a hematopoietic stem cell, a neuron progenitor cell, a neuron, a muscle cell, a bone cell, a hepatocyte, a pancreatic cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, a parenchymal cell, an epithelial cell, an endothelial cell, a mesothelial cell, a fibroblast cell, an osteoblast cell, a chondrocyte cell, an exogenous cell, an endogenous cell, a stem cell, a hematopoietic stem cell, a bone-marrow derived progenitor cell, a myocardial cell, a skeletal cell, a fetal cell, an undifferentiated cell, a multi-potent progenitor cell, a unipotent progenitor cell, a monocyte, a cardiac myoblast, a skeletal myoblast, a macrophage, a capillary endothelial cell, a xenogenic cell, an allogenic cell, an autologous cell, and a post-natal stem cell.

Embodiment III-125. The method of any one of embodiments III-120 to III-124, wherein the modifying occurs in the cells of a subject having a mutation in an allele of a gene wherein the mutation causes a disease or disorder in the subject.

Embodiment III-126. A composition, comprising the engineered CasX protein of any one of embodiments III-42 to III-79.

Embodiment III-127. The composition of embodiment III-126, comprising the ERS of any one of embodiments III-1 to III-41.

Embodiment III-128. The composition of embodiment III-127, wherein the CasX protein and the ERS are associated together in a ribonuclear protein complex (RNP).

Embodiment III-129. A composition, comprising an ERS of any one of embodiments III-1 to III-41.

Embodiment III-130. The composition of embodiment III-129, comprising the engineered CasX protein of any one of embodiments III-42 to III-79.

Embodiment III-131. The composition of embodiment III-130, wherein the engineered CasX protein and the ERS are associated together in a ribonuclear protein complex (RNP).

Embodiment III-132. The composition of any one of embodiments III-127 to III-131, wherein the ERS comprises a targeting sequence of 15 to 20 nucleotides, wherein the targeting sequence is complementary to a target nucleic acid.

Embodiment III-133. The composition of embodiment III-132, wherein the targeting sequence has 20 nucleotides.

Embodiment III-134. A pharmaceutical composition comprising the composition of any one of embodiments III-126-Error! Reference source not found. and a pharmaceutically acceptable excipient.

Embodiment III-135. A pharmaceutical composition comprising the LNP of any one of embodiments III-107 to III-110 and a suitable container.

Embodiment III-136. A kit comprising the pharmaceutical composition of embodiment III-134 or III-135 and a suitable container.

Embodiment III-137. An engineered CasX protein comprising any one of the sequences set forth in SEQ ID NOS: 24916-49628, 49746-49747, and 49871-49873.

Embodiment III-138. An engineered CasX protein comprising any one of the sequences listed in Table 5.

Embodiment III-139. A ERS comprising any one of the ERS sequences selected from the group consisting of SEQ ID NOS: 11,568-22,227 and 23,572-24,915.

Embodiment III-140. The ERS of embodiment III-138, comprising a targeting sequence having 15-20 nucleotides, wherein the targeting sequence is complementary to a target nucleic acid.

Embodiment III-141. The ERS of embodiment III-140, wherein the targeting sequence has 20 nucleotides.

Embodiment III-142. The composition of any one of embodiments III-126—for use in the manufacture of a medicament for the treatment a subject having a disease.

### EXAMPLES

The following Examples are merely illustrative and are not meant to limit any aspects of the present disclosure in any way.

### Example 1: The CcdB Selection Assay Identifies CasX Proteins with dsDNA Nuclease Activity

Experiments were performed to identify a set of highly mutated proteins derived from CasX protein 515 (SEQ ID NO: 228) that are biochemically competent or exhibit improved activity for double-stranded DNA (dsDNA) cleavage at target DNA sequences. To accomplish this, first, a set of sequences was generated using machine learning methods, and second, CcdB selections were performed to validate that the mutated proteins were biochemically competent for dsDNA cleavage.

### Materials and Methods

To engineer novel nucleases composed of many individual single mutations, a Markov Chain Monte Carlo (MCMC)-directed evolution simulation was performed (Biswas S et al. Low-N protein engineering with data-efficient deep learning. Nature Methods. 18(4):389-396 (2021)) where a starting sequence s is mutated to a new sequence s*. First, simulated mutagenesis was performed within the RuvC domain, wherein a codon within CasX 515 was selected and randomly replaced with a codon encoding a different amino acid, such that there was an equal probability of the selected amino acid to be replaced with any of the alternative 19 amino acids. This process was then repeated up to sixteen times, resulting in a simulated mutagenized protein sequence. Second, the predicted fitness of the mutagenized protein sequence was determined using a machine learning model, described below. First, fitness estimates were defined using the following functions: 1) ŷ=f′(s), to estimate the predicted fitness of the starting protein sequence, s; and 2) ŷ*=f′(s*), to estimate the predicted fitness of the simulated mutagenized protein sequence, s*. These predicted fitness estimates were used to virtually screen the simulated protein either to discard the simulated protein or to construct and validate the simulated protein experimentally. To make this decision, an MCMC simulation was performed, where a proposed protein sequence was either accepted or discarded with probability equal to min[1, exp{(ŷ*−ŷ)/T}], where the temperature value T=0.01. Finally, this process of mutagenesis and simulated screening was repeated until a desired number of sequences, each containing a desired number of single mutations, were obtained. Use of the MCMC algorithm resulted in the generation of the following sets of new CasX sequences: 3,600 sequences with two single mutations and 1,200 sequences with three single mutations. Additionally, twenty new sequences were generated for each of the following number of single mutations: 4, 5, 6, 7, 8, 9, 10, 11, 12, 13, 14, 15, 16. Finally, ten additional sequences were generated with 17 single mutations.

In the simulated screening above, the predicted fitness f′ must be calculated for any given sequence s or s*. This fitness prediction was determined using one of three machine learning models, defined as Model A, Model B, or Model C. Model A used the machine learning software Esm1b 0.4.0 (Rives A et al. et al. Biological structure and function emerge from scaling unsupervised learning to 250 million protein sequences. Proc. Natl. Acad. Sci. U.S.A., 181(15):e2016239118 (2021)). To refine Model A, the model was first trained by Support Vector Regression (SVR) using single mutation fitness data. The software package used for SVR was sklearn v.0.22.2.post1 (Pedregosa F et al. Scikit-learn: Machine learning in Python. JMLR 12(85):2825-2830, (2011)). Model B employed the machine learning software TAPE v.0.4 (Rao R et al. Evaluating protein transfer learning with tape. Advances in neural information processing systems, 32:9689 (2019)) and the same dataset was used to fine-tune the pre-trained model. For Model C, the predicted fitness value of a sequence s* was defined as the sum of the true fitness values for each single mutation present in sequence s* relative to sequence s. A custom script was used to calculate predicted fitness values for Model C. Finally, sequences were also generated randomly as negative controls. The predicted fitness score, determined using the three machine learning models for each new CasX sequence designed using simulated mutagenesis, was subsequently used to identify and generate the set of CasX proteins that were subjected to the CcdB bacterial selection experiments as described in the ensuing paragraph.

To obtain the true fitness values for the sequences described above, CcdB bacterial selection experiments were performed. Briefly, 300 ng of p73 plasmid expressing the indicated CasX protein (or library) and gRNA with scaffold 235 with spacer 23.2 (AGAGCGUGAUAUUACCCUGU; SEQ ID NO: 491), which would target the p58 plasmid expressing the CcdB toxin, was electroporated into E. coli strain BW25113 harboring the p58 plasmid. After transformation, the culture was allowed to recover in glucose-rich media for 20 minutes at 37° C. with shaking, after which IPTG was added to a final concentration of 1 mM, and the culture was further incubated for an additional 40 minutes. A recovered culture was then split, where a fraction was titered on LB agar plates containing an antibiotic selective for the plasmid. Cells were titered on plates containing either glucose (CcdB toxin is not expressed) or arabinose (CcdB toxin is expressed), and the relative survival was calculated. The remainder of the recovered culture was further split after the recovery period, and grown in media containing either glucose or arabinose, to collect samples of the pooled library either with no selection or with strong selection, respectively. These cultures were harvested, and the surviving plasmid pool was extracted using a Plasmid Miniprep Kit (QIAGEN) according to the manufacturer's instructions. The entire process was repeated for a total of two rounds of selection. Finally, the entirety of the above protocol was repeated as a “stringent CcdB selection”, using a modified version of the p73 plasmid, specifically, a much weaker promoter (WGAN45) was used to express the guide RNA.

The final plasmid pool was isolated, and a PCR amplification of the p73 plasmid was performed using primers specific for the mutagenized region of CasX 515. Amplified DNA product was purified with Ampure XP DNA cleanup kit and eluted in water. Amplicons were then prepared for sequencing with a second PCR to add adapter sequences compatible with next-generation sequencing (NGS) on either a MiSeq or a NextSeq (Illumina) according to the manufacturer's instructions. Returned raw data files were processed as follows: (1) sequences were trimmed for quality and for adapter sequences; (2) sequences from read 1 and read 2 were merged into a single insert sequence; and (3) each sequence was quantified for containing a mutation relative to the reference sequence for CasX 515. Incidences of individual mutations relative to CasX 515 were counted. Mutation counts post-selection were divided by mutation counts pre-selection, and a pseudocount of ten was used to generate an “Enrichment Score”. The log base two (log 2) of this score was calculated and plotted for the following two sets of sequences: 1) new CasX sequences generated using machine learning and 2) randomly generated sequences used as negative controls.

### Results:

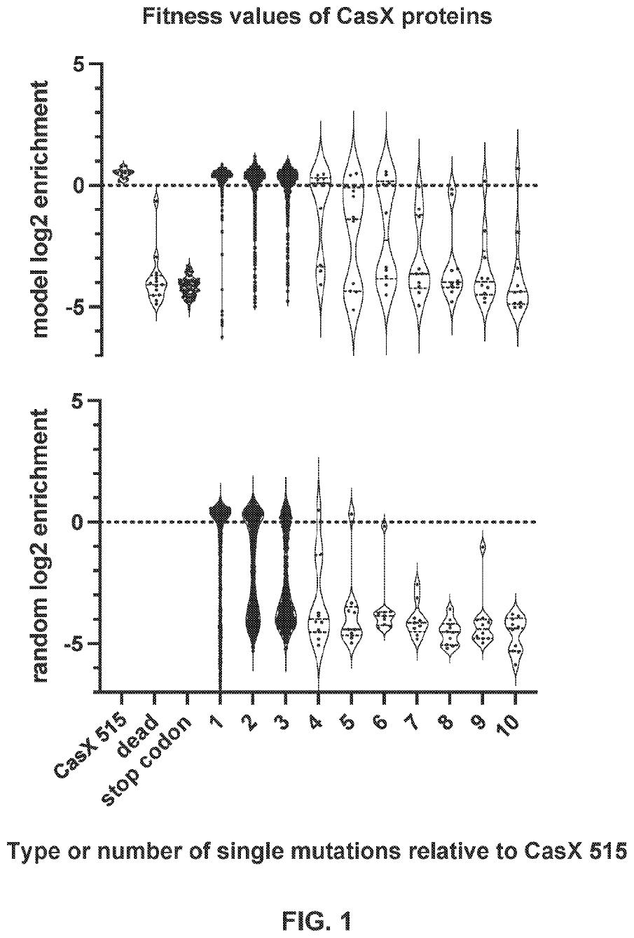

The above described CasX sequences, as well as negative control sequences, were synthesized and cloned as a pooled library, then subjected to two rounds of bacterial selection. The resulting log 2 enrichment scores represented true fitness values, f(s), and were plotted in the graph of FIG. 1. First, 13 sequences of CasX 515 consisting of different codons were plotted. As expected, these sequences produced the same amino acid sequence and all exhibited fitness values of approximately 0.3. In contrast, mutations that either produced catalytically ‘dead’ nucleases or produced stop codons, resulted in CasX proteins with drastically reduced fitness values, ranging from −4 to −6. Alternatively, mutations where one amino acid was exchanged for another resulted in varying effects on fitness, spanning the range from high activity similar to CasX 515, to intermediate or even no levels of activity as seen for catalytically inactivated CasX nucleases. Furthermore, those CasX nucleases harboring mutations designed to maintain function exhibited dramatically improved fitness compared to the fitness level of the randomly mutated CasX molecules (compare top graph with bottom graph in FIG. 1). This trend remained true for CasX nucleases that were designed to contain any number of single mutations between one and ten. In the case where a single mutation was being considered, the average fitness for a model predicted mutation was 0.275, while that for a random mutation was −0.887, and the difference between the distributions was highly statistically significant (p=1.6E-54; two-tailed t-test). Similarly, highly statistically significant p-values were obtained when comparing the difference between the distributions for two (p=8.5E-250; two-tailed t-test) or three mutations (p=5.2E-153; two-tailed t-test). Furthermore, the data illustrate that generation of most CasX sequences containing more than four mutations resulted in substantially poorer fitness values compared to CasX protein sequences with one to three mutations (FIG. 1).

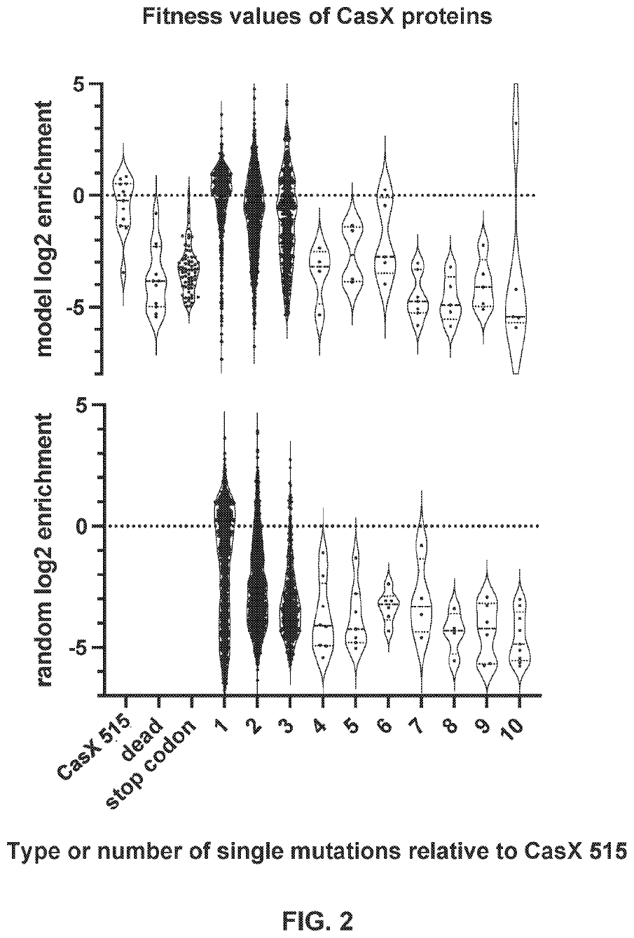

Finally, the above results were validated by repeating the selection under conditions of reduced guide RNA expression, referred to as the stringent CcdB selection, and these enrichment values are plotted in FIG. 2. As illustrated in FIG. 2, the true fitness values determined for the machine-learning derived engineered CasX harboring one to ten mutations relative to CasX 515 were overall higher than the fitness values determined for randomly mutated CasX molecules. Similarly, these distributions were highly statistically significantly different (p=1.9E-48, p=9.6E-116, and p=2.5E-80 for single, double, or triple mutations, respectively; two-tailed t-test). Similarly, the data demonstrate that CasX sequences with more than four mutations produced substantially poor fitness values, mostly ranging from −2 to −6, a fitness level comparable to that observed with the catalytically-dead CasX (FIG. 2). These experiments show that use of machine learning to guide design of CasX molecules resulted in the generation of significantly improved nucleases compared to nucleases generated via random mutation. Those nucleases with activity greater than or comparable to CasX 515, that is, with average true fitness value determined to be >0 (for n=3 biological replicates), are provided in SEQ ID NOS: 24916-27856.

### Example 2: Individual Mutations Conferring CasX Proteins with Improved Biochemical Properties can be Combined to Further Improve Properties

Experiments were performed to identify single mutations (e.g., amino acid substitutions, insertions, or deletions) and combinations of single mutations that would improve one or more of the following biochemical properties of the CasX protein: (1) exhibit improved specificity for cleavage at on-target versus off-target sites in human cells; (2) utilize alternative protospacer adjacent motif (PAM) recognition sequences other than the canonical PAM sequence ‘TTC’; and (3) show improved nuclease activity of the CasX protein. To accomplish this, the HEK293 cell line PASS_V1.03 was treated with the WT CasX protein 2 (SEQ ID NO: 2) or with mutated CasX proteins, and next-generation sequencing (NGS) was performed to calculate the percent editing at a variety of spacers and associated target sites.

### Materials and Methods

A multiplexed pooled approach was taken to assay clonal proteins derived from CasX 515 listed in Table 9 using a pooled activity and specificity (PASS) assay. Here, a pooled HEK cell line, which was adapted to suspension culture from adherent cells, was generated and termed PASS_V1.03. Methods to complete the production of the PASS_V1.03 line were previously described in International Publication No. WO2022120095A1, incorporated herein by reference.

TABLE 9
List of CasX proteins derived from CasX
515 assessed here using the PASS assay

| | Mutation(s) applied to CasX 515 | Amino acid |
|---|---|---|
| CasX protein no. | (Position.Reference.Alternative)* | SEQ ID NO |
| CasX 591 | 292.V.L | 492 |
| CasX 593 | 304.M.W | 493 |
| CasX 844 | 292.V.L + 304.M.W | 494 |
| CasX 532 | 27.—.R | 495 |
| CasX 535 | 224.G.S | 496 |
| CasX 668 | 27.—.R + 224.G.S | 497 |
| CasX 946 | 701.E.H | 498 |
| CasX 947 | 709.K.Y | 499 |
| CasX 948 | 701.E.H + 709.K.Y | 500 |

*Positions of mutations are shown relative to a CasX 515 sequence with an N-terminal methionine residue (SEQ ID NO: 49699).

To assess the editing activity and specificity of a CasX nuclease at human target sites, two sets of target sites were quantified. First, 47 TTC PAM on-target sites, where the 20 nucleotides of the spacer were perfectly complementary to the target site, were quantified, and the average editing efficiency and standard error of the mean across this set of target sites was calculated for two biological replicates. Second, 91 TTC PAM off-target sites were quantified, where each spacer-target pair consisted of a single nucleotide mismatch at one of the twenty positions of the target site. The average editing efficiency and standard error of the mean for two biological replicates were also calculated for this set of target sites. Similarly, average editing efficiency was calculated for sets of target sites with alternative PAMs, as described in International Publication No. WO2022120095A1. Finally, CcdB bacterial selections were performed, and log 2 enrichment values were calculated as described in International Publication No. WO2022120095A1.

### Results:

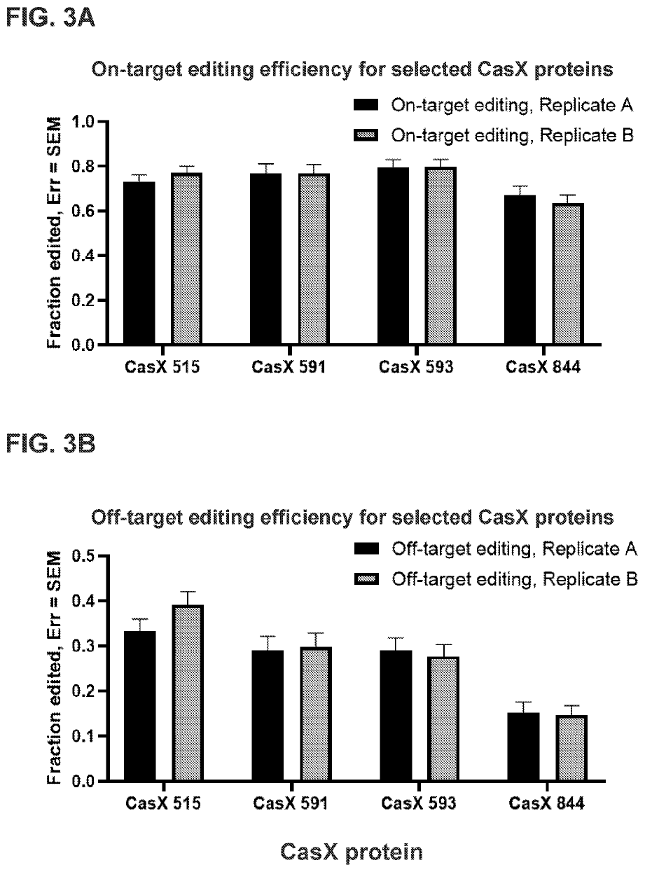

The average on-target editing activity and average off-target editing activity were determined for the following CasX proteins: CasX 515; two single-mutation proteins derived from CasX 515 (i.e., CasX 591 and 593); and CasX 844, which contains both of the single mutations combined. Results showing average on-target editing activity and average off-target editing activity are illustrated in bar plots in FIG. 3A and FIG. 3B respectively. The data show that on average, CasX 515 was able to edit ˜75% of the on-target sites and ˜36% of the off-target sites. In contrast, while CasX 591 and 593 were able to maintain similar on-target editing rates (average˜77% and 80% respectively), their average off-target editing rates dropped to 30% and 28%, respectively. Finally, the combined double mutant CasX 844 was able to edit on-target sites at an average rate of 65%, which is ˜10% lower in comparison to the average editing rate achieved by CasX 515. However, the off-target editing rates for CasX 844 were substantially lower, achieving an average off-target rate of ˜15%. Taken together, while CasX 844 exhibited a small loss of on-target editing activity relative to that achieved by CasX 515, it was able to edit less than 50% of the number of off-target sites targeted by CasX 515. This is an indication that improvements to specificity conferred by each single mutation could be synergistically combined, resulting in enhanced CasX editing specificity.

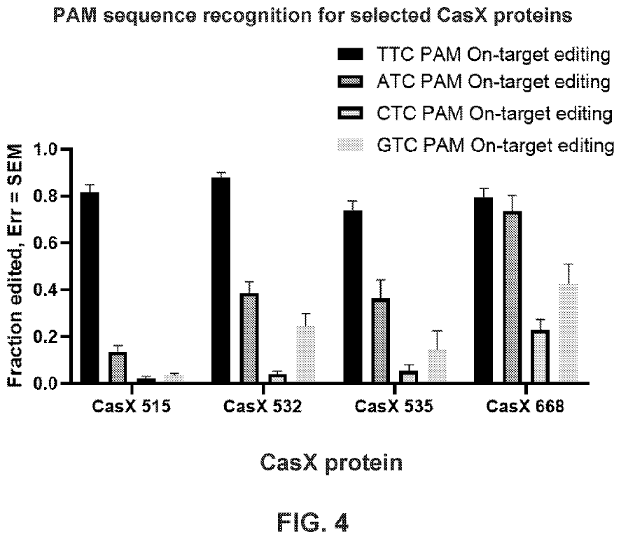

To assess the synergistic or additive quality of combining individual mutations experimentally, PASS data were generated for additional sets of target sites with alternative PAM sequences beyond the wild-type TTC sequence for CasX proteins 515, 532, 535, and 668 (see Table 10 for specific mutations relative to CasX 515), using the method described in International Publication No. WO2022120095A1. FIG. 4 is a bar plot showing the average on-target editing activity of the four CasX proteins across a series of four different PAM sequences. Similarly, the synergistic effect of combining multiple single mutations was observed, here, in the context of increased recognition of three novel PAM sequences as indicated. For each non-TTC PAM sequence, each of the CasX proteins having two single mutations exhibited increased editing level relative to the level attained with CasX 515. When the two mutations were combined to generate CasX 668, a synergistic increase in editing level was observed for the non-canonical PAM sequences, especially with CTC and GTC PAM.

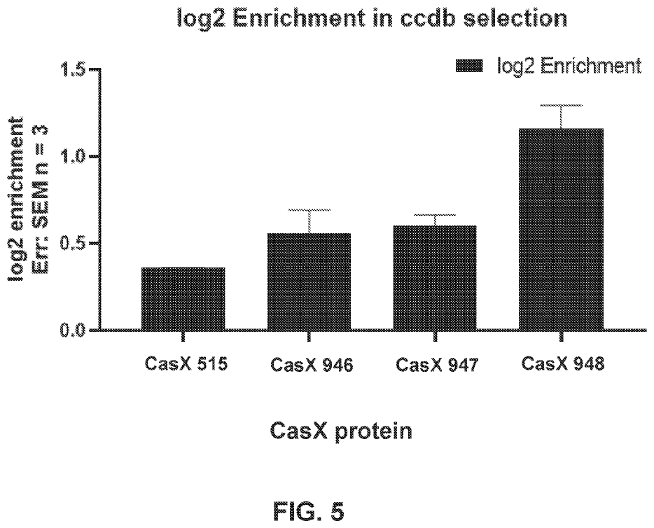

Next, the CcdB bacterial selection assay was employed to assess nuclease activity from CasX 515, two derivatives of CasX 515 with a different single mutation (CasX 946 and CasX 947), and a CasX 515 derivative harboring both mutations (CasX 948). The results, represented as the mean log 2 enrichment value from three biological replicates, are shown in FIG. 5 for the indicated CasX. The results show that relative to the nuclease activity demonstrated by CasX 515, the K683Y mutation of CasX 947 resulted in a statistically significant increase in activity (p=0.019; two-tailed t-test), and that the E675H of CasX 946 mutation resulted in seemingly improved, though not statistically significantly different, activity (FIG. 5). Furthermore, combining the two single mutations in CasX 948 dramatically and significantly increased the log 2 enrichment score from that obtained for CasX 515, from an average of 0.359 to an average of 1.16, with a significance value of p=0.0096 by two tailed t-Test.

These biochemical properties are thus subject to synergistic or additive effects of combinatorial single mutations. Table 10 lists the single mutations shown to improve on-target TTC PAM editing activity relative to CasX 515 in at least one biological replicate in the PASS assay. Table 11 lists the single mutations demonstrated to improve editing specificity relative to CasX 515 in at least one biological replicate in the PASS assay. Table 12 lists the single mutations determined to alter the PAM recognition sequence of CasX 515 in at least one biological replicate in the PASS assay.

Structural Insights into the Observed Improvements in Activity, Specificity, and/or PAM Recognition of CasX Proteins:

A structural analysis of various mutations revealed additional insights that could explain the observed improvement in specificity of the tested CasX proteins. In the case of CasX 591, the substitution of a valine to a leucine at position 292 might have resulted in additional bulk around the duplex helix of the R-loop, since leucine contains a single additional carbon in its hydrophobic side chain relative to that of valine. This increased bulk might have resulted in increased structural constraint of the R-loop, thereby prohibiting mismatch-induced distortions to occur in the R-loop. Similarly, the improved specificity demonstrated by CasX 593 might be attributed to the substitution of a tryptophan for a methionine at position 304. The large side chain of tryptophan possibly contributed to the increased bulk proximal to the gRNA:DNA duplex. Furthermore, other mutations might improve specificity via a different mechanism. CasX 812 was generated via a glycine-to-lysine substitution at position 329, within the Helical I-II domain, while CasX 594 was generated via a glycine-to-asparagine substitution at the same position (see summary of the CasX 812 sequence in Table 13, below). Both substitutions at position 329 appeared to improve specificity, which could be explained by two potential mechanisms. First, removing glycine could reduce the flexibility and therefore enhance structural rigidity in this region of the protein, thereby impeding the formation of mismatches between the gRNA and target DNA and increasing nuclease specificity. Second, adding lysine or asparagine might induce additional hydrogen bonding between these side chains and the gRNA. Such interactions might enforce an “A-form” geometry on the RNA, thereby hindering accommodating structural changes that would allow mismatches to exist within the R-loop. Finally, some mutations may improve specificity by destabilizing the overall R-loop. Such destabilizing effects might be sufficient to prevent R-loop formation at less stable mismatched off-target sites, while the more stable, fully complementary on-target site would remain fully capable of R-loop formation. For instance, in the case of CasX 757, the loss of the positively charged lysine as a result of the lysine-to-glutamine substitution at position 796 might reduce the protein's binding affinity for the negatively-charged DNA backbone of the proximal non-target strand, thereby destabilizing the R-loop. Similarly, the lysine-to-glutamine substitution at position 611 to generate CasX 824 might improve specificity by removing excess stabilizing energy mediated by ionic interactions with the backbone of the proximal DNA target strand, thereby preferentially reducing off-target effects. Similarly, in the case of CasX 781, the lysine-to-glutamate substitution at position 390 resulted in a stronger destabilization effect: here, the attractive positive charge of the lysine was replaced with a repulsive negative charge of a glutamate, thereby destabilizing interactions with the target strand.

In the case of the improved PAM recognition effects seen with CasX proteins 532, 533, and 668 (FIG. 4), the additive behavior of the two mutations tested (27.-.R and 224.G.S) was potentially enabled by the fact that the two mutations might improve access to new PAM sequences via two different biochemical mechanisms. First, position 224 in CasX 515 (or position 223 when excluding a prepended methionine) was identified as an important modifier of nucleotide preference at the first position of the PAM sequence. The glycine-to-serine substitution resulting in CasX 535 improved PAM recognition at all three non-canonical PAMs (ATC, CTC, and GTC). It is possible that this improved recognition occurred due to additional bonding interactions between the edge of the nucleotide base, which was physically proximal to this position, and the amino acid side-chain of the serine residue. Additionally, replacing glycine for serine might be stabilizing the alpha-helix in this region. Stabilizing this alpha-helix would reduce the energy required for R-loop formation, therefore unlocking the editing activity at non-canonical PAM sequences that were slightly imperfectly recognized. Furthermore, it is possible that the alpha carbon of glycine was capable of Van der Waals interactions with the methyl group of the thymine nucleobase, and these interactions were potentially abrogated upon substituting glycine for serine. Finally, there might be other amino acid positions (e.g., position 230) within CasX 515 capable of recognizing certain nucleotides within the PAM recognition sequence of CasX. Second, the insertion of an arginine at position 27, as was the case in CasX 532, was previously observed to be involved in broadening PAM preference. This insertion, located in a loop of the OBD domain proximal to the non-target strand, might be mediating additional ionic interactions between the positively charged side-chain and negatively charged DNA backbone of the non-target strand. As a result, this interaction might be involved in stabilizing the R-loop regardless of the specific nucleotide in position 1 of the PAM sequence, thereby improving editing efficiency. Furthermore, there might be additional positions within CasX capable of accepting a positively charged amino acid and remaining physically close to the DNA backbone to mediate stabilization of the unwound DNA.

Lastly, the improved nuclease activity of some CasX proteins might be attributed to an increased stability of the unwound R-loop state. For instance, in the case of CasX 583, a hydrophobic leucine was replaced by a positively charged lysine at position 169. The lysine side chain would be free to interact with the proximal negatively charged backbone of the non-target DNA strand, thereby stabilizing the R-loop. Similarly, an insertion of a positively charged arginine at position 27, as was the case in CasX 532, would be expected to increase the stability of the unwound R-loop state by interacting with the proximal negatively charged backbone of the DNA target strand. Furthermore, additional affinity of the CasX protein for the gRNA would increase the effective concentration of active RNP, which would increase the overall editing rate. This might be how CasX 818, which contained a serine-to-arginine substitution at position 698, demonstrated improved nuclease activity, since the arginine would be physically proximal to the scaffold region of the gRNA. CasX 643, in which glutamine was replaced with a phenylalanine at position 593, might also be demonstrating improved editing in a similar manner, i.e., by improving the affinity of the CasX protein for the gRNA through base-stacking between the phenylalanine and proximal cytosine at position 19 of the gRNA. Finally, in the case of CasX 654, the replacement of a methionine by a serine at position 772 would result in additional hydrogen bonding with the minor groove of the R-loop duplex or with the backbone of the non-target DNA strand.

Overall, the results here demonstrate that the biochemical properties of nuclease activity, nuclease specificity, and PAM recognition ability of CasX proteins can be improved or altered by multiple single mutations acting in combination to achieve greater effects than any single mutation alone.

TABLE 10
Single mutations resulting in average on-target TTC PAM
editing activity greater than that achieved by CasX 515
in at least one biological replicate of the PASS assay

| | | | Difference in average |
|---|---|---|---|
| | Mutation relative | CasX | on-target TTC PAM |
| CasX | to CasX 515* | amino acid | editing activity |
| protein | (Position.Refer- | sequence | relative to CasX |
| no. | ence.Alternative) | (SEQ ID NO) | 515 (fraction) |
| 532 | 27.—.R | 495 | 0.08 |
| 535 | 224.G.S | 49629 | 0.10 |
| 555 | 171.A.D | 49630 | 0.06 |
| 559 | 4.I.G | 49631 | 0.22 |
| 561 | 5.—.G | 49632 | 0.01 |
| 562 | 5.K.G | 49633 | 0.13 |
| 564 | 6.—.G | 49634 | 0.05 |
| 566 | 7.I.A | 49635 | 0.05 |
| 568 | 8.N.S | 49636 | 0.11 |
| 569 | 9.K.G | 49637 | 0.03 |
| 572 | 35.R.P | 49638 | 0.04 |
| 573 | 53.E.P | 49639 | 0.12 |
| 577 | 64.R.Q | 49640 | 0.14 |
| 580 | 86.W.D | 49641 | 0.10 |
| 583 | 169.L.K | 49642 | 0.16 |
| 584 | 171.A.Y | 49643 | 0.11 |
| 585 | 171.A.S | 49644 | 0.06 |
| 587 | 224.G .— | 49645 | 0.03 |
| 590 | 289.K.S | 49646 | 0.09 |
| 591 | 292.V.L | 49647 | 0.14 |
| 592 | 304.M.T | 49648 | 0.20 |
| 593 | 304.M.W | 493 | 0.16 |
| 602 | 339.—.Q | 49649 | 0.01 |
| 604 | 342.—.A | 49650 | 0.002 |
| 607 | 398.Y.T | 49651 | 0.02 |
| 613 | 412.—.E | 49652 | 0.002 |
| 638 | 481.E.D | 49653 | 0.02 |
| 643 | 593.Q.F | 49654 | 0.03 |
| 644 | 593.Q.V | 49655 | 0.02 |
| 646 | 653.—.T | 49656 | 0.06 |
| 649 | 655.—.S | 49657 | 0.002 |
| 651 | 696.G.R | 26039 | 0.03 |
| 654 | 772.M.S | 49658 | 0.05 |
| 656 | 887.T.D | 49659 | 0.005 |
| 702 | 91.K.V | 49660 | 0.01 |
| 718 | 7.I.L | 49661 | 0.05 |
| 721 | 156.F.V | 49662 | 0.04 |
| 736 | 794.P.A | 49663 | 0.05 |
| 760 | 797.T.V | 49664 | 0.10 |
| 762 | 793.—.P | 49665 | 0.06 |
| 780 | 389.—.V | 49666 | 0.04 |
| 787 | 826.V.M | 49667 | 0.04 |
| 788 | 891.S.Q | 49668 | 0.04 |
| 789 | 917.G.E | 49669 | 0.04 |
| 790 | 951.—.S | 49670 | 0.04 |
| 791 | 953.—.K | 49671 | 0.04 |
| 812 | 329.G.K | 266 | 0.05 |
| 818 | 698.S.R | 272 | 0.05 |
| 824 | 611.K.Q | 278 | 0.23 |

*Positions of mutations are shown relative to a CasX 515 sequence with an N-terminal methionine residue (SEQ ID NO: 49699).

TABLE 11
Single mutations resulting in average off-target TTC PAM editing
activity less than that achieved by CasX 515 in at least one
biological replicate of the PASS assay, and where the average
on-target TTC PAM editing rate is not lower than that achieved
by CasX 515 by more than an absolute value of 10%

| | Mutation relative | CasX | Average off-target |
|---|---|---|---|
| CasX | to CasX 515* | amino acid | TTC PAM |
| protein | (Position.Refer- | sequence | editing activity |
| no. | ence.Alternative) | (SEQ ID NO) | (fraction) |
| 544 | 232.D.G | 49672 | 0.34 |
| 555 | 171.A.D | 49630 | 0.30 |
| 562 | 5.K.G | 49633 | 0.29 |
| 564 | 6.—.G | 49634 | 0.33 |
| 568 | 8.N.S | 49636 | 0.27 |
| 569 | 9.K.G | 49637 | 0.27 |
| 572 | 35.R.P | 49638 | 0.21 |
| 580 | 86.W.D | 49641 | 0.23 |
| 584 | 171.A.Y | 49643 | 0.23 |
| 585 | 171.A.S | 49644 | 0.24 |
| 587 | 224.G.— | 49645 | 0.14 |
| 590 | 289.K.S | 49646 | 0.22 |
| 591 | 292.V.L | 49647 | 0.25 |
| 593 | 304.M.W | 493 | 0.19 |
| 594 | 329.G.N | 49673 | 0.12 |
| 607 | 398.Y.T | 49651 | 0.34 |
| 609 | 405.L.N | 49674 | 0.24 |
| 610 | 405.L.W | 49675 | 0.19 |
| 611 | 408.E.Y | 49676 | 0.24 |
| 612 | 412.G.P | 49677 | 0.25 |
| 613 | 412.—.E | 49652 | 0.34 |
| 614 | 414.—.R | 49678 | 0.33 |
| 616 | 414.—.Y | 49679 | 0.31 |
| 619 | 417.K.D | 49680 | 0.33 |
| 622 | 420.D.G | 49681 | 0.22 |
| 631 | 469.L.K | 49682 | 0.21 |
| 632 | 469.L.S | 49683 | 0.24 |
| 633 | 473.—.D | 49684 | 0.25 |
| 638 | 481.E.D | 49653 | 0.26 |
| 643 | 593.Q.F | 49654 | 0.33 |
| 644 | 593.Q.V | 49655 | 0.35 |
| 649 | 655.—.S | 49657 | 0.27 |
| 657 | 893.—.N | 49685 | 0.27 |
| 702 | 91.K.V | 49660 | 0.24 |
| 717 | 390.K.Q | 49686 | 0.20 |
| 718 | 7.I.L | 49661 | 0.28 |
| 721 | 156.F.V | 49662 | 0.28 |
| 757 | 796.K.Q | 49687 | 0.25 |
| 758 | 791.E.N | 49688 | 0.22 |
| 777 | 169.L.Q | 49689 | 0.24 |
| 779 | 372.G.I | 49690 | 0.19 |
| 780 | 389.—.V | 49666 | 0.29 |
| 781 | 390.K.E | 49691 | 0.24 |
| 784 | 570.P.I | 49692 | 0.22 |
| 788 | 891.S.Q | 49668 | 0.27 |
| 789 | 917.G.E | 49669 | 0.25 |
| 790 | 951.—.S | 49670 | 0.21 |
| 791 | 953.—.K | 49671 | 0.23 |
| 812 | 329.G.K | 266 | 0.16 |
| 818 | 698.S.R | 272 | 0.30 |
| 824 | 611.K.Q | 278 | 0.16 |

*Positions of mutations are shown relative to a CasX 515 sequence with an N-terminal methionine residue (SEQ ID NO: 49699).

TABLE 12
Single mutations resulting in log2 enrichment >0 when
selected against synthetic PAM sequences of ATC
or CTC in the CedB bacterial selection.

| | Mutation relative | CasX | | | |
|---|---|---|---|---|---|
| CasX | to CasX 515* | amino acid | log2 | | |
| protein | (Position.Refer- | sequence | en- | | |
| no. | ence.Alternative) | (SEQ ID NO) | richment | Spacer | PAM |
| 528 | 224.G.Y | 49693 | 9.09 | 23.19 | ATC |
| 534 | 224.G.H | 49694 | 4.11 | 23.27 | CTC |
| 535 | 224.G.S | 496 | 8.31 | 23.19 | ATC |
| 536 | 224.G.T | 49695 | 3.81 | 11.2 | CTC |
| 537 | 224.G.A | 49696 | 6.29 | 11.2 | CTC |
| 538 | 224.G.V | 49697 | 3.62 | 23.27 | CTC |
| 583 | 169.L.K | 49642 | 4.21 | 11.2 | CTC |
| 587 | 224.G.— | 49645 | 3.94 | 23.27 | CTC |
| 532 | 27.—.R | 495 | 2.96 | 11.2 | CTC |
| 949 | 231.S.R | 49698 | 8.95 | 24.25 | TTG |

*Positions of mutations are shown relative to a CasX 515 sequence with an N-terminal methionine residue (SEQ ID NO: 49699).

TABLE 13
CasX 812 domain sequences and coordinates

| | SEQ | | |
|---|---|---|---|
| Domain | ID NO | Amino Acid Sequence* | Coordinates |
| N-terminal | — | M | 1 |
| methionine | | | |
| OBD-I | 295 | QEIKRINKIRRRLVKDSNTKKAGKTGPMKTLLVRVMTPDLRERLEN | 2-57 |
| | | LRKKPENIPQ | |
| Helical I-I | 296 | PISNTSRANLNKLLTDYTEMKKAILHVYWEEFQKDPVGLMSRVA | 58-101 |
| NTSB | 297 | QPASKKIDQNKLKPEMDEKGNLTTAGFACSQCGQPLFVYKLEQVSE | 102-192 |
| | | KGKAYTNYFGRCNVAEHEKLILLAQLKPEKDSDEAVTYSLGKFGQ | |
| Helical I-II | 49847 | RALDFYSIHVTKESTHPVKPLAQIAGNRYASGPVGKALSDACMGTI | 193-333 |
| | | ASFLSKYQDIIIEHQKVVKGNQKRLESLRELAGKENLEYPSVTLPP | |
| | | QPHTKEGVDAYNEVIARVRMWVNLNLWQKLKLSRDDAKPLLRLKKF | |
| | | PSF | |
| Helical II | 299 | PLVERQANEVDWWDMVCNVKKLINEKKEDGKVFWQNLAGYKRQEAL | 334-501 |
| | | RPYLSSEEDRKKGKKFARYQLGDLLLHLEKKHGEDWGKVYDEAWER | |
| | | IDKKVEGLSKHIKLEEERRSEDAQSKAALTDWLRAKASFVIEGLKE | |
| | | ADKDEFCRCELKLOKWYGDLRGKPFAIEAE | |
| OBD-II | 300 | NSILDISGFSKQYNCAFIWQKDGVKKLNLYLIINYFKGGKLRFKKI | 502-647 |
| | | KPEAFEANRFYTVINKKSGEIVPMEVNENFDDPNLIILPLAFGKRQ | |
| | | GREFIWNDLLSLETGSLKLANGRVIEKTLYNRRTRQDEPALFVALT | |
| | | FERREVLD | |
| RuvC-I | 301 | SSNIKPMNLIGVDRGENIPAVIALTDPEGCPLSRFKDSLGNPTHIL | 648-811 |
| | | RIGESYKEKORTIQAKKEVEQRRAGGYSRKYASKAKNLADDMVRNT | |
| | | ARDLLYYAVTQDAMLIFENLSRGFGRQGKRTEMAERQYTRMEDWLT | |
| | | AKLAYEGLPSKTYLSKTLAQYTSKTC | |
| TSL | 302 | SNCGETITSADYDRVLEKLKKTATGWMTTINGKELKVEGQITYYNR | 812-921 |
| | | YKRONVVKDLSVELDRLSEESVNNDISSWTKGRSGEALSLLKKRFS | |
| | | HRPVQEKFVCLNCGFETH | |
| RuvC-II | 303 | ADEQAALNIARSWLFLRSQEYKKYQTNKTTGNTDKRAFVETWQSFY | 922-979 |
| | | RKKLKEVWKPAV | |

*Position of the 329.G.K mutation relative to CasX 515 is bolded and underlined.

### Example 3: The PASS Assay Identifies CasX Proteins with Enhanced Specificity Relative to CasX 515

Experiments were performed to identify CasX proteins with improved specificity for cleavage at on-target versus off-target sites in human cells. To accomplish this, the HEK293 cell line PASS_V1.03 was treated with the WT CasX protein 2 or with CasX proteins with mutations relative to WT, and next-generation sequencing (NGS) was performed to calculate the percent editing at a variety of spacers and associated target sites.

### Materials and Methods

The multiplexed pooled PASS assay was used, as described in Example 2. Samples were treated in biological triplicate, as well as technical duplicate, for a total of six replicates for each CasX protein. Here, the CasX proteins tested were wild-type CasX 2, engineered CasX proteins 119, 491, 515, 593, and 812; Streptococcus pyogenes (“Spy”) Cas9 served as a negative control.

To assess the editing activity and specificity of a CasX nuclease at human target sites, two sets of target sites were quantified. First, TTC PAM on-target sites, where the twenty nucleotides of each gRNA spacer targeting these on-target sites were perfectly complementary to the target site, were quantified. For each sample and spacer-target pair, data based on <500 reads were removed. Fraction indel values for each sample and spacer-target pair were subtracted by the average fraction indel value across Cas9-treated samples with the same spacer-target pair, where Cas9 served as a negative control due to the absence of a compatible guide RNA. Second, TTC PAM off-target sites, where one of the twenty nucleotides of the spacer was mismatched with the target site, were quantified. As above, for each sample and spacer-target pair, data based on <500 reads were removed, and fraction indel values for each sample and spacer-target pair were subtracted by the average fraction indel value across Cas9-treated samples with the same spacer-target pair. Finally, for those TTC PAM spacer-target pairs that had both an on-target and an off-target version, the average editing activity and 95% confidence intervals were calculated.

### Results:

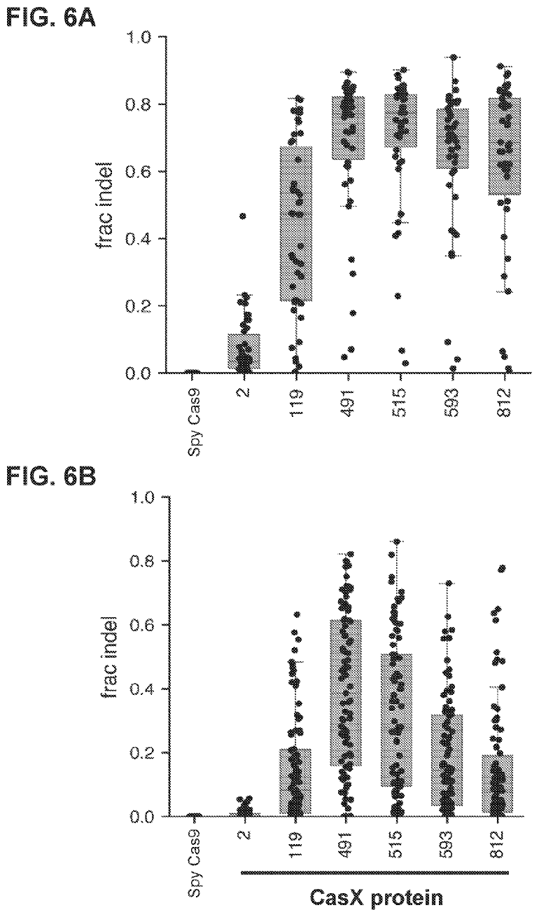

The average on-target editing activity and average off-target editing activity were determined for the following CasX proteins: the wild-type protein CasX 2, the CasX proteins 119, 491, and 515, and two single-mutation proteins derived from CasX 515 (i.e., CasX 593 and 812). Cas9 was also included as a negative control, where no editing was expected due to the lack of a compatible gRNA. Results showing average on-target editing activity and average off-target editing activity are illustrated as box plots in FIG. 6A and FIG. 6B, respectively. The data show that on average, CasX 812 was able to edit on-target sites at a high rate, on average˜93% as efficiently as CasX 515 (FIG. 6A), yet CasX 812 demonstrated, on average, a 2.7× lower rate of editing at off-target sites compared with that exhibited by CasX 515 (FIG. 6B). These data show that when compared with CasX 515, CasX 812 is, on average, nearly as effective at editing at a chosen target site while exhibiting substantially reduced off-target editing, an important safety consideration for their use as therapeutics.

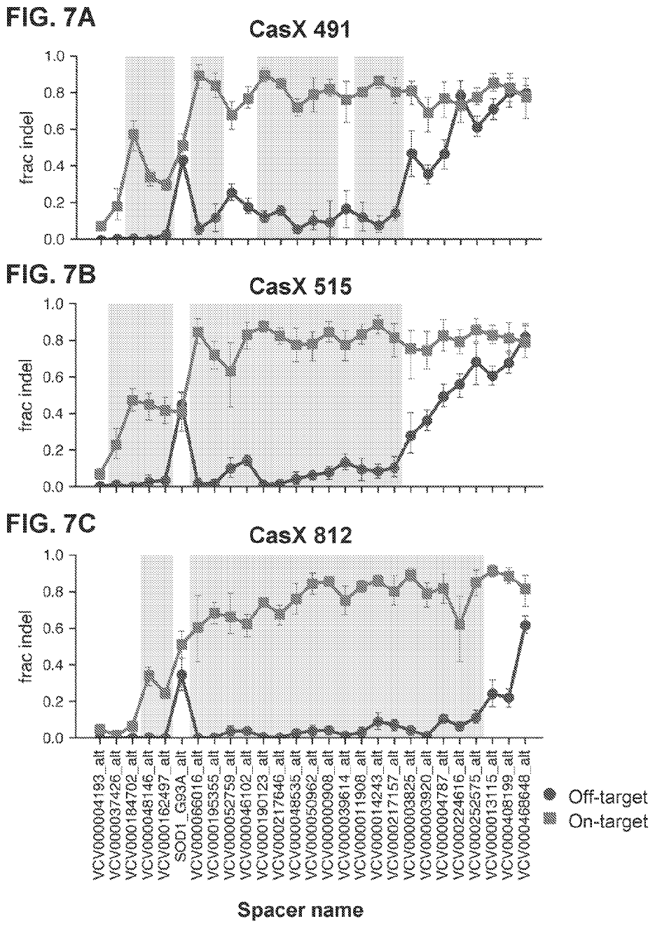

While the average editing rate across spacers is a useful metric to compare CasX proteins, individual spacers often produce different editing levels. FIGS. 7A-7C are point plots showing the editing rates for select CasX proteins using gRNA with 27 different human sequence spacers. For each spacer, the editing rate is shown for both the on-target and off-target site, where the off-target site consists of a human single nucleotide polymorphism. The results show that some individual spacers can be classified as allele-specific, where the on-target editing rate is greater than 20%, and the off-target editing rate is less than one-fifth of the on-target editing rate. Furthermore, the number of spacers that can be classified as allele-specific depends on the CasX protein being used: for CasX 491, 13 of these spacers met these criteria (FIG. 7A; regions highlighted in gray are allele-specific). In contrast, use of CasX 515 resulted in 17 allele-specific spacers meeting the criterion (FIG. 7B), while CasX 812 resulted in 20 allele-specific spacers (FIG. 7C). Taken together, these data show that, compared to CasX 515, CasX 812 has an improved average specificity across spacers, as well as an improved number of allele-specific spacers.

The results here demonstrate that the nuclease specificity of CasX proteins can be improved or altered by single mutations, and that CasX proteins 593 and 812 have improved specificity relative to the control protein CasX 515.

### Example 4: CasX:gRNA In Vitro Cleavage Assays

Experiments were performed to assess in vitro DNA cleavage by CasX:gRNA ribonucleoproteins (RNPs).

### Materials and Methods

### Assembly of RNP

RNPs of either CasX 119, CasX 491, CasX 515 (SEQ ID NO: 228), or CasX 812 (SEQ ID NO: 266) were assembled with single guide RNAs (sgRNA) with scaffold 316 (SEQ ID NO: 156) and one of two spacers, as described in detail below. The amino acid sequences of CasX 119 and CasX 491 are disclosed in International Publication No. WO2020247882A1. Separately, RNPs of CasX 515 were assembled with sgRNA with either scaffold 2 (SEQ ID NO: 5), 174 (SEQ ID NO: 17), 235 (SEQ ID NO: 75), or 316 and one of two spacers.

Purified RNP of CasX and sgRNA were prepared same-day prior to experiments. For experiments where protein variants were being compared, the CasX protein was incubated with sgRNA at 1:1.2 molar ratio. When scaffolds were compared, the protein was added in 1.2:1 ratio to guide. Briefly, sgRNA was added to Buffer #1 (20 mM Tris HCl pH 7.5, 150 mM NaCl, 1 mM TCEP, 5% glycerol, 10 mM MgCl₂) on ice, then CasX was added to the sgRNA solution, slowly with swirling, and immediately incubated at 37° C. for 20 minutes to form RNP complexes. RNP complexes were centrifuged at 4° C. for 5 minutes at 16,000×g to remove any precipitate. Formation of competent (active) RNP was assessed as described below. In vitro cleavage assays

The ability of CasX variants to form active RNP compared to reference CasX was determined using an in vitro cleavage assay. The beta-2 microglobulin (B2) 7.9 and 7.37 target for the cleavage assay was created as follows. DNA oligos (sequences in Table 14) were generated with 5′ terminal amino modification for conjugation to Cy-dyes with an amino-reactive handle (N-hydroxysuccinimide). Oligo-dye conjugation reactions of 100 uM oligo and 1 mM dye were performed in 100 mM sodium borate pH 8.3 at 4° C. for 16 h. Target strands (TS) were labeled with Cy5.5 and non-targeting strands (NTS) were labeled with Cy7.5. After quenching the reactions with 1 mM Tris pH 7.5, the conjugated oligos were purified via ethanol precipitation. Double-stranded DNA (dsDNA) targets were formed by mixing the oligos in a 1:1 ratio in 1× hybridization buffer (20 mM Tris HCl pH 7.5, 100 mM KCl, 5 mM MgCl₂), heating to 95° C. for 10 minutes, and allowing the solution to cool to room temperature.

TABLE 14
DNA sequences and descriptions of target DNAs

| DNA Sequence (5′-3′)* | SEQ ID NO: | Description |
|---|---|---|
| /5AmMC6/TGAAGCTGACAGCATTCGGGCCGAGA | 49874 | 7.37 NTS with 5' amine for NHS ester |
| TGTCTCGCTCCGTGGCCTTAGCTGTGCTCGCGCT | | linkages** |
| /5AmMC6/AGCGCGAGCACAGCTAAGGCCACGGA | 49875 | 7.37 TS with 5' amine ** |
| GCGAGACATCTCGGCCCGAATGCTGTCAGCTTCA | | |
| /5AmMC6/TGAAGCTGACAGCATTCGGGCCTAGA | 49876 | 7.37 gel probe NTS, mismatch at 5 |
| TGTCTCGCTCCGTGGCCTTAGCTGTGCTCGCGCT | | |
| /5AmMC6/AGCGCGAGCACAGCTAAGGCCACGGA | 49877 | 7.37 gel probe TS, mismatch at 5 |
| GCGAGACATCTAGGCCCGAATGCTGTCAGCTTCA | | |
| /5AmMC6/TGAAGCTGACAGCATTCGGGCCGAGA | 49878 | 7.37 gel probe NTS, mismatch at 10 |
| TATCTCGCTCCGTGGCCTTAGCTGTGCTCGCGCT | | |
| /5AmMC6/AGCGCGAGCACAGCTAAGGCCACGGA | 49879 | 7.37 gel probe TS, mismatch at 10 |
| GCGAGATATCTCGGCCCGAATGCTGTCAGCTTCA | | |
| /5AmMC6/TGAAGCTGACAGCATTCGGGCCGAGA | 49880 | 7.37 gel probe NTS, mismatch at 15 |
| TGTCTCGATCCGTGGCCTTAGCTGTGCTCGCGCT | | |
| /5AmMC6/AGCGCGAGCACAGCTAAGGCCACGGA | 49881 | 7.37 gel probe TS, mismatch at 15 |
| TCGAGACATCTCGGCCCGAATGCTGTCAGCTTCA | | |
| /5AmMC6/CTTTCAGCAAGGACTGGTCTTTCTAT | 49882 | 7.9 target TS |
| CTCTTGTACTACACTGAATTCACCCCCACTGAAA | | |
| /5AmMC6/TTTCAGTGGGGGTGAATTCAGTGTAG | 49883 | 7.9 target NTS |
| TACAAGAGATAGAAAGACCAGTCCTTGCTGAAAG | | |
| /5AmMC6/CTTTCAGCAAGGACTGGTCTTTCTAT | 49884 | 7.9 gel probe TS, mismatch at 5 |
| CTCTTGTACGACACTGAATTCACCCCCACTGAAA | | |
| /5AmMC6/TTTCAGTGGGGGTGAATTCAGTGTCG | 49885 | 7.9 gel probe NTS, mismatch at 5 |
| TACAAGAGATAGAAAGACCAGTCCTTGCTGAAAG | | |
| /5AmMC6/CTTTCAGCAAGGACTGGTCTTTCTAT | 49886 | 7.9 gel probe TS, mismatch at 10 |
| CTCTCGTACTACACTGAATTCACCCCCACTGAAA | | |
| /5AmMC6/TTTCAGTGGGGGTGAATTCAGTGTAG | 49887 | 7.9 gel probe NTS, mismatch at 10 |
| TACGAGAGATAGAAAGACCAGTCCTTGCTGAAAG | | |
| /5AmMC6/CTTTCAGCAAGGACTGGTCTTTCTGT | 49888 | 7.9 gel probe TS, mismatch at 15 |
| CTCTTGTACTACACTGAATTCACCCCCACTGAAA | | |
| /5AmMC6/TTTCAGTGGGGGTGAATTCAGTGTAG | 49889 | 7.9 gel probe NTS, mismatch at 15 |
| TACAAGAGACAGAAAGACCAGTCCTTGCTGAAAG | | |

*5AmMC6 indicates the 5′ Amino Modifier C6. The target sequences are underlined.
**The Kcleave assay using the mismatched position 5 dsDNA target was run at 37° C.

### Determining Cleavage-Competent Fractions for RNPs

Cleavage reactions were prepared with final RNP concentrations of 100 nM and final target concentration of 100 nM. Reactions were carried out at 37° C. and initiated by the addition of the dye-labeled dsDNA target. Aliquots were taken at 5, 30, and 60 minutes and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software.

### Kcleave Assay

Cleavage reactions were set up with a final RNP concentration of 200 nM and a final target concentration of 10 nM. Reactions were carried out at 16° C., except where otherwise noted, and initiated by the addition of the target DNA. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX:sgRNA combination replicate individually.

To test the relative specificities of engineered proteins in vitro, apparent cleavage rate constants were compared for targets with mismatched bases at various positions (5, 10, and 15 nt downstream of PAM, Table 14). Cleavage assays were performed in large excess of RNP (200 nM RNP and 1 nM target dsDNA) at 16° C., with the exception of assays measuring cleavage of the target with a mismatch at 5 nt, which were conducted at 37° C. in order to observe measurable cleavage rates. Aliquots were taken at 15, 30, 60, 120, 180, 240, and 480 seconds, and quenched by adding to 95% formamide, 25 mM EDTA. Samples were denatured by heating at 95° C. for 10 minutes and run on a 10% urea-PAGE gel. The gels were imaged with a Cytiva Typhoon and quantified using the Cytiva IQTL software. The apparent first-order rate constant of non-target strand cleavage (k_{cleave}) was determined for each CasX:sgRNA combination replicate individually.

### Results:

### Determining Cleavage-Competent Fractions for Protein Variants Compared to Reference CasX 119

To determine the cleavage-competent fraction for the tested CasX proteins, it was assumed that CasX acts essentially as a single-turnover enzyme under the assayed conditions, as indicated by the observation that sub-stoichiometric amounts of enzyme fail to cleave a greater-than-stoichiometric amount of target even under extended time-scales and instead approach a plateau that scales with the amount of enzyme present. Thus, the fraction of target cleaved over long time-scales by an equimolar amount of RNP is indicative of what fraction of the RNP is properly formed and active for cleavage. Thus, the active (competent) fraction for each RNP was derived from the cleaved fraction over the total signal at the 60-minute timepoint, upon confirming an increase in cleaved fraction from the 5-minute timepoint, and relative plateau in cleaved fraction from the 30-minute timepoint.

Apparent competent fractions were determined for the RNPs with various CasX proteins, and are provided in Table 15.

TABLE 15
Protein variant RNP comparison of fraction competence and Kcleave rates

| | | Kcleave (sec⁻¹) | | | |
|---|---|---|---|---|---|
| | Fraction | On- | Position 15 | Position 10 | Position 5 |
| RNP | competence* | target | mismatch | mismatch | mismatch |
| (CasX.Scaffold.Spacer) | (mean ± stdev) | spacer | spacer | spacer | spacer** |
| 119.174.7.9 | 26.71 ± 9.65% | 0.0292 | 0.0158 | 0.0047 | 0.0127 |
| 491.174.7.9 | 49.23 ± 6.37% | 0.0966 | 0.0552 | 0.03 | 0.0616 |
| 515.174.7.9 | 30.15 ± 3.58% | 0.0782 | 0.0625 | 0.0324 | 0.0392 |
| 812.174.7.9 | 53.28 ± 5.14% | 0.0909 | 0.0512 | 0.0142 | 0.01 |
| 119.174.7.37 | 29.01 ± 2.67% | 0.0022 | 0.0061 | 0.0028 | 0.0034 |
| 491.174.7.37 | 48.73 ± 5.44% | 0.0425 | 0.015 | 0.0297 | 0.0301 |
| 515.174.7.37 | 43.54 ± 16.19% | 0.0396 | 0.0127 | 0.0297 | 0.0347 |
| 812.174.7.37 | 38.23 ± 2.49% | 0.027 | 0.0111 | 0.0119 | 0.014 |

*Active fraction was calculated by averaging three experimental replicates.
**The Kcleave assay using the mismatched position 5 dsDNA target was run at 37° C.

For protein variant comparison, the following CasX proteins were used with guide scaffold 316 and spacer 7.9 or guide 316 and spacer 7.37: CasX 119, CasX 491, CasX 515, and CasX 812. CasX 119 had the lowest active fraction for both spacers, indicating that CasX 491, CasX 515, and CasX 812 form more active and stable RNP with the identical guides under the tested conditions as compared to CasX 119. CasX proteins 491, 515, and 812 did not show consistent trends in their competent fractions across the two spacers, consistent with the expectation that the additional engineering following CasX 491 primarily affects target engagement and cleavage, rather than guide binding or stability.

Kcleave Assay to Understand Specificity of RNPs Formed from Protein Variants

Assays were performed to measure the apparent first-order rate constant of non-target strand cleavage (k_{cleave}), and the results are presented in Table 15, above. A drastic effect on the kinetics of CasX 812 RNP cleavage was observed for on-target versus the mismatched dsDNA target for both spacers. CasX 812 had comparable on-target cleavage rates to CasX 491 and CasX 515 for both spacers, with a slightly higher cleavage rate than 515 on spacer 7.9, which might be explained by the lower competent fraction observed for the 515 RNP with that spacer, and a lower cleavage rate on 7.37.

The off-target rates for CasX 812 were much more substantially reduced for most of the mismatched substrates. The difference in kcleave rates was readily apparent for the target with a mismatch at position 10, with 812 having a roughly 6-fold (7.9) and 2-fold (7.37) reduction in cleavage rate, as compared to its on-target rate. CasX 515, by comparison, exhibited a 2.4-fold and a 25% reduction on the same targets. A substantial difference was also observed for the position 5 mismatch targets. Even though the assay was run at 37° C. to enable measurable cleavage rates, as the position 5 mismatch targets were essentially uncleaved by the CasX RNPs at the lower temperature used for the other targets, CasX 812 against spacer 7.9 exhibited a 9-fold reduction in cleavage rate from on-target rate run at 16° C. and a 2-fold reduction for the 7.37 spacer with a position 5 mismatch. CasX 515 showed a 2-fold reduction for mismatched 7.9 and a nearly equivalent cleavage rate for 7.37 with the position 5 mismatch (note that the “equivalent” cleavage rate is due to the increased temperature).

For the position 15 mismatch substrate, CasX 812 exhibited modest reductions in cleavage rates relative to on-target rates, comparable to the reduction observed for 515. This suggests that the increased sensitivity of CasX 812 to mismatches declines by the PAM distal region, at least for the specific mismatches and spacers tested here. The increased sensitivity at positions 5 and 10 in particular correlates with the position of the G329K mutation present in CasX 812. This mutation introduces a positive charge near the RNA spacer around position 8 and may help CasX to better read out distortions caused by mismatches. Mismatches closer to this new site of contact would be more likely to significantly disrupt either R-loop propagation or allosteric activation of the RuvC (depending on the precise mechanism of increased specificity), while mismatches farther away (as in the position 15 mismatch) might have more variable effects depending on the nature of the mismatch and its effects on the broader heteroduplex structure. Taken together, these data confirm that CasX 812 is inherently more sensitive to mismatches between the RNA spacer and the DNA target and is not simply a less active enzyme, as the decrease in cleavage rate at mismatched targets is in excess of the decrease in cleavage rate at properly matched targets. This is consistent with the results in Examples 2, 6, and 7 that indicate that CasX 812 is a highly specific enzyme, with lower off-target editing compared to the other nucleases tested.

### Determining Cleavage-Competent Fractions for Single Guide Variants Relative to Reference Single Guide 2

RNPs were complexed using the aforementioned methods. To isolate the effect of sgRNA identity on RNP formation, guide-limiting conditions were employed. sgRNAs with scaffolds 2, 174, 235, or 316 with spacers 7.9 or 7.37 were mixed with CasX 515 at final concentrations of 1 μM for the guide and 1.2 μM for the protein. Fraction competence was calculated as described above, and the results are provided in Table 16.

TABLE 16
Guide variant RNP comparison of fraction
competence and Kcleave assay

| RNP construct | Competent fraction | |
|---|---|---|
| (CasX.Scaffold.Spacer) | (mean ± stdev) | Kcleave (sec⁻¹) |
| 515.2.7.9 | 28.14 6.87% | 0.1346 ± 0.0118 |
| 515.174.7.9 | 42.77 9.62% | 0.1723 ± 0.0046 |
| 515.235.7.9 | 34.11 1.15% | 0.1696 ± 0.0571 |
| 515.316.7.9 | 30.89 6.87% | 0.1413 ± 0.0301 |
| 515.2.7.37 | 10.42 2.24% | 0.0204 ± 0.0002 |
| 515.174.7.37 | 19.96 2.88% | 0.0534 ± 0.0200 |
| 515.235.7.37 | 32.64 11.60% | 0.0647 ± 0.0163 |
| 515.316.7.37 | 26.98 11.08% | 0.0851 ± 0.0071 |

* active fraction was calculated by averaging two experimental replicates

Given the complex folding structure of the CasX guide, fraction competence is expected to largely be determined by how much of the guide is properly folded for interaction with the protein. All guides with engineered scaffolds showed improvements over scaffold 2, and guides with scaffold 235 or ERS 316 showed improvements relative to 174 for spacer 7.37. This is consistent with the introduction of mutations in the pseudoknot and triplex that are expected to stabilize the properly folded form. As described in Example 12, below, scaffold 235 and ERS 316 both produced higher levels of gene editing than scaffold 174 when assayed in a cell culture system and delivered via lentiviral vectors at a relatively low multiplicity of infection.

Higher competent fractions of all guides were observed for spacer 7.9. For this spacer, scaffold 174 had the highest competent fraction, followed by scaffolds 316, 235, and 2. Proper guide folding is expected to be highly dependent on the potential for undesired interactions between the scaffold and spacer sequences, so the observed differences may be attributable to differential sequence-specific interactions, variations in prep quality, or noise in the assay.

Determining k_{Cleave} for Single Guide Variants Compared to Reference Scaffold 2

Cleavage assays were performed with CasX 515 and guides with reference scaffold 2 compared to guides with scaffolds 174, 235, or 316 with spacer 7.9 or 7.37 to determine relative cleavage rates. The mean and standard deviation of three replicates with independent fits are presented in Table 16, above.

To reduce cleavage kinetics to a range measurable with the assay, the cleavage reactions were incubated at 16° C. Under these conditions, all guides supported faster cleavage rates as compared to scaffold 2. For spacer 7.37, the cleavage kinetics aligned with those guides that contributed to the highest fraction competence, with the highest cleavage rate being sg174 (0.1723 s⁻¹), followed by scaffold 235 (0.1696 s⁻¹) and scaffold 316 (0.1413 s⁻¹), versus scaffold 2 (0.1346 s⁻¹). For spacer 7.9, scaffold 316 yielded the highest cleavage rate (0.0851 s⁻¹), followed by scaffold 235 (0.0647 s⁻¹) and sg174 (0.0534 s⁻¹), versus scaffold 2 (0.0204 s⁻¹). The fraction competence and k_{cleave} data did not demonstrate differences across the engineered variants that were consistent across both spacers, although all are consistently better than scaffold 2. This suggests that the improvements seen for scaffold 235 and 316 over 174 are primarily due to behavior in the cell, whether it be stability in the cytoplasm, folding in the cytoplasm, transcription when delivered via plasmid or AAV, or refolding ability when delivered via LNP, that are not captured by guides that have been in vitro transcribed, refolded, and tested for cleavage biochemically.

### Example 5: The PASS Assay Identifies Engineered CasX Proteins with Enhanced Activity, Specificity, and/or PAM Recognition Relative to CasX 515

Experiments are performed to assay engineered CasX proteins designed with a combination of mutations introduced in CasX 515 to identify engineered CasX proteins exhibiting improvements in the following types of biochemical properties: 1) editing activity; 2) editing activity and specificity for cleavage at on-target versus off-target sites; 3) editing activity and PAM recognition; and 4) editing activity, specificity, and PAM recognition. To accomplish this, the HEK293 cell line PASS_V1.03 is treated with the wild-type CasX protein 2 (SEQ ID NO: 2) or with CasX protein 515 (SEQ ID NO: 228), or an engineered CasX protein, and next-generation sequencing (NGS) is performed to calculate the percent editing at a variety of spacers and associated target sites.

### Materials and Methods

To engineer CasX nucleases with improved activity, specificity, and/or PAM recognition, individual single mutations are combined to generate the library of CasX protein sequences provided in SEQ ID NOS: 27857-49628.

Single mutations were identified above to improve activity (see Table 10, Example 2), specificity (see Table 11, Example 2), or PAM recognition (see Table 12, Example 2) relative to CasX 515. The following types of combinations of single mutations are generated to produce new engineered CasX: 1) activity+activity; 2) activity+specificity; 3) activity+activity+PAM recognition; and 4) activity+specificity+PAM recognition. The resulting engineered CasX proteins are constructed and assayed using the PASS system, a multiplexed pooled approach described in Example 2.

To assess the editing activity and specificity of an engineered CasX protein at human target sites, editing at two sets of target sites is quantified. First, editing at TTC PAM on-target sites, where the twenty nucleotides of each gRNA spacer targeting these on-target sites are perfectly complementary to the target site, is quantified, and the average editing efficiency and standard error of the mean for two biological replicates are calculated for this set of on-target sites. Second, editing at TTC PAM off-target sites, where each spacer-target pair consists of a single nucleotide mismatch at one of the 20 positions of the target site, is quantified. The average editing efficiency and standard error of the mean is calculated for this set of target sites. The ratio of editing efficiency, as well as the propagated standard error of the mean, between the off-target and the on-target sites is calculated. This ratio metric is defined as the Specificity Ratio.

To assess PAM sequence specificity for each engineered CasX protein, average editing efficiency and standard error of the mean is similarly calculated for sets of target sites with non-canonical PAM sequences ATC, CTC, and GTC.

The results from these experiments are expected to identify those engineered CasX proteins harboring a combination of mutations designed to improve the following relative to the CasX 515 control: 1) double-strand cleavage activity; 2) editing activity and specificity for cleavage at on-target versus off-target sites; 3) editing activity and PAM recognition; and 4) editing activity, specificity, and PAM recognition. Specifically, the results are expected to reveal those engineered CasX proteins with higher average editing efficiency at target DNA sequences associated with TTC PAM, as well as engineered CasX proteins with a higher specificity ratio (measured as editing at on-target versus off-target sites). The data is also expected to reveal those engineered CasX proteins with higher average editing efficiency at target DNA sequences with alternative PAM sequences (ATC, CTC, or GTC). These data are expected to demonstrate that a broad spectrum of engineered CasX proteins can be engineered with improved biochemical properties having enhanced activity and specificity for a specific therapeutic target of interest.

### Example 6: Identification of CasX Proteins with Enhanced Activity or Specificity Relative to CasX 515

An experiment was performed to identify CasX proteins with single mutations and increased editing activity or improved specificity relative to CasX 515.

### Materials and Methods

The multiplexed pooled PASS assay was used as described in Example 2. CasX proteins were expressed using a relatively weakly-expressing promoter to reduce CasX protein expression and thereby improve the sensitivity of the assay. Samples were tested in quadruplicate. The list of CasX proteins tested and their mutations relative to CasX 515 is provided in Tables 17 and 18, below. All of the tested CasX proteins had single mutations (i.e., a single amino acid substitution, deletion, or insertion) relative to CasX 515, except for CasX 676, which has three mutations relative to CasX 515. Streptococcus pyogenes Cas9 without a guide RNA served as a negative control.

As described in Example 3, to assess the editing activity and specificity of the tested CasX proteins at human target sites, two sets of target sites were quantified. First, editing was quantified at TTC PAM on-target sites in which the twenty nucleotides of each gRNA spacer targeting these on-target sites were perfectly complementary to the target site. For each sample and spacer-target pair, data based on <500 reads were removed. Fraction indel values for each sample and spacer-target pair were subtracted by the average fraction indel value across Cas9-treated samples with the same spacer-target pair; Cas9 served as a negative control due to the absence of a compatible guide RNA. Second, editing was quantified at TTC PAM off-target sites, in which one of the twenty nucleotides of the spacer was mismatched with the target site. As above, for each sample and spacer-target pair, data based on <500 reads were removed, and fraction indel values for each sample and spacer-target pair were subtracted by the average fraction indel value across Cas9-treated samples with the same spacer-target pair. Finally, for those TTC PAM spacer-target pairs that had both an on-target and an off-target version, the average editing activity and standard error of the mean (SEM) were calculated.

### Results:

Table 17 provides the level of on-target editing produced by various CasX proteins with mutations relative to CasX 515, ranked from highest to lowest activity.

TABLE 17
Average on-targeting editing activity,
ranked from highest to lowest

| | | Average | SEM on-target |
|---|---|---|---|
| Protein | Mutation relative to | on-target TTC | TTC PAM |
| Designation | CasX 515* | PAM editing | editing |
| (CasX protein | (Position.Refer- | activity | activity |
| no., or Cas9) | ence.Alternative) | (fraction) | (fraction) |
| 607 | 398.Y.T | 2.72E−01 | 4.08E−02 |
| 532 | 27.—.R | 2.59E−01 | 3.34E−02 |
| 676 | 27.—.R & 170.L.K | 2.36E−01 | 3.42E−02 |
| | & 224.G.S | | |
| 592 | 304.M.T | 2.19E−01 | 3.73E−02 |
| 788 | 891.S.Q | 2.18E−01 | 3.43E−02 |
| 583 | 169.L.K | 2.17E−01 | 3.66E−02 |
| 555 | 171.A.D | 2.14E−01 | 3.73E−02 |
| 515 | — | 2.09E−01 | 3.82E−02 |
| 569 | 9.K.G | 2.08E−01 | 3.12E−02 |
| 787 | 826.V.M | 2.07E−01 | 3.45E−02 |
| 561 | 5.—.G | 1.98E−01 | 3.41E−02 |
| 577 | 64.R.Q | 1.94E−01 | 3.48E−02 |
| 585 | 171.A.S | 1.94E−01 | 3.40E−02 |
| 572 | 35.R.P | 1.89E−01 | 3.95E−02 |
| 536 | 224.G.T | 1.88E−01 | 3.43E−02 |
| 656 | 887.T.D | 1.87E−01 | 3.34E−02 |
| 559 | 4.I.G | 1.87E−01 | 3.22E−02 |
| 777 | 169.L.Q | 1.84E−01 | 3.36E−02 |
| 584 | 171.A.Y | 1.83E−01 | 3.66E−02 |
| 779 | 372.G.I | 1.79E−01 | 3.15E−02 |
| 566 | 7.I.A | 1.77E−01 | 3.37E−02 |
| 638 | 481.E.D | 1.77E−01 | 3.37E−02 |
| 593 | 304.M.W | 1.76E−01 | 3.35E−02 |
| 568 | 8.N.S | 1.76E−01 | 3.34E−02 |
| 562 | 5.K.G | 1.74E−01 | 3.04E−02 |
| 564 | 6.—.G | 1.73E−01 | 3.32E−02 |
| 757 | 796.K.Q | 1.73E−01 | 2.94E−02 |
| 654 | 772.M.S | 1.70E−01 | 3.18E−02 |
| 760 | 797.T.V | 1.70E−01 | 3.12E−02 |
| 818 | 698.S.R | 1.67E−01 | 3.31E−02 |
| 646 | 653.—.T | 1.66E−01 | 3.06E−02 |
| 784 | 570.P.I | 1.65E−01 | 2.64E−02 |
| 762 | 793.—.P | 1.64E−01 | 2.99E−02 |
| 789 | 917.G.E | 1.61E−01 | 2.91E−02 |
| 649 | 655.—.S | 1.58E−01 | 3.13E−02 |
| 594 | 329.G.N | 1.56E−01 | 3.26E−02 |
| 604 | 342.—.A | 1.55E−01 | 2.87E−02 |
| 612 | 412.G.P | 1.55E−01 | 2.81E−02 |
| 644 | 593.Q.V | 1.52E−01 | 2.97E−02 |
| 736 | 794.P.A | 1.52E−01 | 2.85E−02 |
| 790 | 951.—.S | 1.51E−01 | 2.87E−02 |
| 780 | 389.—.V | 1.49E−01 | 3.02E−02 |
| 717 | 390.K.Q | 1.43E−01 | 2.84E−02 |
| 633 | 473.—.D | 1.41E−01 | 2.71E−02 |
| 590 | 289.K.S | 1.40E−01 | 2.92E−02 |
| 657 | 893.—.N | 1.39E−01 | 2.96E−02 |
| 643 | 593.Q.F | 1.38E−01 | 2.80E−02 |
| 544 | 232.D.G | 1.37E−01 | 3.19E−02 |
| 591 | 292.V.L | 1.37E−01 | 2.76E−02 |
| 534 | 224.G.H | 1.36E−01 | 2.24E−02 |
| 791 | 953.—.K | 1.34E−01 | 2.60E−02 |
| 781 | 390.K.E | 1.33E−01 | 2.71E−02 |
| 718 | 7.I.L | 1.32E−01 | 2.85E−02 |
| 812 | 329.G.K | 1.29E−01 | 2.97E−02 |
| 609 | 405.L.N | 1.26E−01 | 2.57E−02 |
| 758 | 791.E.N | 1.24E−01 | 2.33E−02 |
| 616 | 414.—.Y | 1.22E−01 | 2.44E−02 |
| 632 | 469.L.S | 1.22E−01 | 2.79E−02 |
| 614 | 414.—.R | 1.18E−01 | 2.32E−02 |
| 721 | 156.F.V | 1.14E−01 | 2.48E−02 |
| 611 | 408.E.Y | 1.11E−01 | 2.08E−02 |
| 622 | 420.D.G | 1.07E−01 | 2.41E−02 |
| 610 | 405.L.W | 1.06E−01 | 2.21E−02 |
| 619 | 417.K.D | 1.05E−01 | 2.59E−02 |
| 580 | 86.W.D | 1.00E−01 | 2.22E−02 |
| 602 | 339.—.Q | 9.82E−02 | 2.53E−02 |
| 537 | 224.G.A | 9.21E−02 | 2.34E−02 |
| 587 | 224.G.— | 8.18E−02 | 2.19E−02 |
| 538 | 224.G.V | 7.16E−02 | 2.16E−02 |
| 702 | 91.K.V | 6.31E−02 | 1.93E−02 |
| 824 | 611.K.Q | 4.07E−02 | 1.23E−02 |
| 631 | 469.L.K | 3.01E−02 | 1.30E−02 |
| 573 | 53.E.P | 2.65E−02 | 1.11E−02 |
| 528 | 224.G.Y | 8.98E−03 | 7.97E−03 |
| 535 | 224.G.S | 7.92E−03 | 7.31E−03 |
| Cas9 | n/a | 7.17E−03 | 8.11E−03 |

*Positions of mutations are shown relative to a CasX 515 sequence with an N-terminal methionine residue (SEQ ID NO: 49699). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

As shown in Table 17, CasX proteins 607, 532, 676, 592, 788, 583, and 555 produced higher levels of on-target editing than did CasX 515. CasX proteins 569, 787, 561, 577, 585, and 572 also produced relatively high levels of on-target editing, with at least 90% of the activity of CasX 515 (i.e., greater than 1.88E-01 on-target editing).

Table 18 provides the level of off-target editing produced by various CasX proteins with mutations relative to CasX 515, ranked from lowest to highest activity.

TABLE 18
Average off-targeting editing activity,
ranked from lowest to highest

| | | Average | SEM |
|---|---|---|---|
| Protein | Mutation relative | off-target | off-target |
| Designation | to CasX 515* | TTC PAM | TTC PAM |
| (CasX | (Position. | editing | editing |
| protein no., | Reference. | activity | activity |
| or Cas9) | Alternative) | (fraction) | (fraction) |
| 528 | 224.G.Y | 1.73E−03 | 1.84E−03 |
| Cas9 | n/a | 2.18E−03 | 2.72E−03 |
| 535 | 224.G.S | 2.33E−03 | 2.63E−03 |
| 573 | 53.E.P | 5.01E−03 | 3.29E−03 |
| 824 | 611.K.Q | 5.86E−03 | 2.93E−03 |
| 631 | 469.L.K | 6.23E−03 | 3.45E−03 |
| 587 | 224.G.— | 1.15E−02 | 4.68E−03 |
| 538 | 224.G.V | 1.45E−02 | 7.45E−03 |
| 702 | 91.K.V | 1.52E−02 | 6.87E−03 |
| 812 | 329.G.K | 1.58E−02 | 7.16E−03 |
| 580 | 86.W.D | 1.78E−02 | 6.27E−03 |
| 619 | 417.K.D | 1.94E−02 | 6.80E−03 |
| 610 | 405.L.W | 2.08E−02 | 6.80E−03 |
| 758 | 791.E.N | 2.11E−02 | 7.51E−03 |
| 721 | 156.F.V | 2.12E−02 | 6.94E−03 |
| 591 | 292.V.L | 2.15E−02 | 7.81E−03 |
| 537 | 224.G.A | 2.18E−02 | 8.82E−03 |
| 590 | 289.K.S | 2.28E−02 | 8.23E−03 |
| 622 | 420.D.G | 2.30E−02 | 7.70E−03 |
| 632 | 469.L.S | 2.34E−02 | 7.92E−03 |
| 633 | 473.—.D | 2.36E−02 | 8.67E−03 |
| 614 | 414.—.R | 2.38E−02 | 7.57E−03 |
| 594 | 329.G.N | 2.41E−02 | 9.14E−03 |
| 643 | 593.Q.F | 2.46E−02 | 8.93E−03 |
| 609 | 405.L.N | 2.47E−02 | 8.24E−03 |
| 781 | 390.K.E | 2.48E−02 | 8.34E−03 |
| 616 | 414.—.Y | 2.48E−02 | 7.29E−03 |
| 602 | 339.—.Q | 2.55E−02 | 9.70E−03 |
| 791 | 953.—.K | 2.71E−02 | 7.83E−03 |
| 593 | 304.M.W | 2.87E−02 | 1.04E−02 |
| 644 | 593.Q.V | 2.88E−02 | 9.11E−03 |
| 657 | 893.—.N | 2.89E−02 | 1.02E−02 |
| 717 | 390.K.Q | 2.99E−02 | 1.00E−02 |
| 611 | 408.E.Y | 3.00E−02 | 8.86E−03 |
| 572 | 35.R.P | 3.06E−02 | 1.07E−02 |
| 780 | 389.—.V | 3.07E−02 | 9.83E−03 |
| 818 | 698.S.R | 3.15E−02 | 1.02E−02 |
| 638 | 481.E.D | 3.16E−02 | 1.01E−02 |
| 584 | 171.A.Y | 3.39E−02 | 1.07E−02 |
| 790 | 951.—.S | 3.47E−02 | 1.02E−02 |
| 718 | 7.I.L | 3.47E−02 | 1.06E−02 |
| 649 | 655.—.S | 3.54E−02 | 1.09E−02 |
| 562 | 5.K.G | 3.56E−02 | 1.09E−02 |
| 784 | 570.P.I | 3.60E−02 | 9.80E−03 |
| 736 | 794.P.A | 3.61E−02 | 1.03E−02 |
| 789 | 917.G.E | 3.63E−02 | 1.11E−02 |
| 544 | 232.D.G | 3.64E−02 | 1.15E−02 |
| 612 | 412.G.P | 3.64E−02 | 1.12E−02 |
| 604 | 342.—.A | 3.66E−02 | 1.11E−02 |
| 564 | 6.—.G | 3.67E−02 | 1.07E−02 |
| 568 | 8.N.S | 3.91E−02 | 1.15E−02 |
| 779 | 372.G.I | 3.98E−02 | 1.20E−02 |
| 760 | 797.T.V | 3.99E−02 | 1.09E−02 |
| 777 | 169.L.Q | 4.11E−02 | 1.16E−02 |
| 566 | 7.I.A | 4.20E−02 | 1.15E−02 |
| 569 | 9.K.G | 4.29E−02 | 1.03E−02 |
| 577 | 64.R.Q | 4.38E−02 | 1.28E−02 |
| 536 | 224.G.T | 4.44E−02 | 1.32E−02 |
| 656 | 887.T.D | 4.54E−02 | 1.22E−02 |
| 646 | 653.—.T | 4.56E−02 | 1.21E−02 |
| 757 | 796.K.Q | 4.60E−02 | 1.25E−02 |
| 559 | 4.I.G | 4.71E−02 | 1.19E−02 |
| 585 | 171.A.S | 4.73E−02 | 1.24E−02 |
| 515 | — | 4.85E−02 | 1.33E−02 |
| 762 | 793.—.P | 4.91E−02 | 1.25E−02 |
| 561 | 5.—.G | 4.92E−02 | 1.37E−02 |
| 788 | 891.S.Q | 5.50E−02 | 1.45E−02 |
| 555 | 171.A.D | 5.53E−02 | 1.52E−02 |
| 787 | 826.V.M | 5.68E−02 | 1.33E−02 |
| 654 | 772.M.S | 5.84E−02 | 1.61E−02 |
| 583 | 169.L.K | 5.95E−02 | 1.48E−02 |
| 592 | 304.M.T | 5.97E−02 | 1.48E−02 |
| 534 | 224.G.H | 6.37E−02 | 1.52E−02 |
| 676 | 27.—.R & 170.L.K | 7.13E−02 | 1.68E−02 |
| | & 224.G.S | | |
| 607 | 398.Y.T | 8.25E−02 | 1.82E−02 |
| 532 | 27.—.R | 8.70E−02 | 1.73E−02 |

*Positions of mutations are shown relative to a CasX 515 sequence with an N-terminal methionine residue (SEQ ID NO: 49699). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

As shown in Table 18, many of the tested CasX proteins showed lower levels of off-target editing than did CasX 515. For example, consistent with the results presented in Example 2, CasX 812 again produced relatively low levels of off-target editing. Further, some of the tested CasX proteins showed even lower levels of off-target editing than did CasX 812 (specifically, CasX 528, 535, 573, 824, 631, 587, 538, and 702)

Based on these results, a set of mutation conferring a high degree of editing activity and/or specificity was chosen for introducing in pairs into CasX 515. First, high activity mutations were defined as those that showed a level of on-target editing equal to at least 87.3% of the level of on-target editing by CasX 515. CasX 607, 532, 676, 592, 788, 583, 555, 569, 787, 561, 577, 585, 572, 536, 656, 559, 777, and 584 met this threshold, and were therefore selected as potential activity-enhancing mutations (see Table 19). Second, high specificity mutations were defined as those producing 80% or lower of the level of off-target editing produced by CasX 515, while maintaining at least 79.95% of the on-target editing activity of CasX 515. This 80% on-target editing activity requirement was implemented to avoid selecting mutations that were simply loss-of-function mutations and would therefore not be expected to be useful as gene editors. CasX 593, 572, 818, 638, 584, 562, and 784 met these criteria, and were therefore selected as potential specificity-enhancing mutations (see Table 19).

In total, 22 individual mutations were chosen as candidates for introducing in pairs into CasX 515 and testing for improved properties, as described in Example 7, below. The positions of the individual mutations relative to full-length CasX 515 protein, as well as amino acid sequences of full-length CasX proteins with the individual mutations, are provided in Table 19. Table 20, below, shows the amino acid sequences and coordinates of the domains of CasX 515, and Table 21 shows the positions of the 22 individual mutations within the domains of CasX 515, as well as the amino acid sequences of domains with each individual mutations.

TABLE 19
Summary of positions of single mutations
within the CasX 515 protein

| | | Mutation relative | Full-length |
|---|---|---|---|
| | | to CasX 515* | CasX |
| CasX | | (Position. | amino acid |
| protein | | Reference. | sequence |
| no. | Phenotype | Alternative) | (SEQ ID NO) |
| 532 | Activity | 27.—.R | 495 |
| 536 | Activity | 224.G.T | 49695 |
| 555 | Activity | 171.A.D | 49630 |
| 559 | Activity | 4.I.G | 49631 |
| 561 | Activity | 5.—.G | 49632 |
| 562 | Specificity | 5.K.G | 49633 |
| 564 | Specificity | 6.—.G | 49634 |
| 569 | Activity | 9.K.G | 49637 |
| 572 | Activity | 35.R.P | 49638 |
| | (and specificity) | | |
| 577 | Activity | 64.R.Q | 49638 |
| 583 | Activity | 169.L.K | 49640 |
| 584 | Activity | 171.A.Y | 49642 |
| | (and specificity) | | |
| 585 | Activity | 171.A.S | 49643 |
| 592 | Activity | 304.M.T | 49643 |
| 593 | Specificity | 304.M.W | 493 |
| 607 | Activity | 398.Y.T | 49651 |
| 638 | Specificity | 481.E.D | 49653 |
| 656 | Activity | 887.T.D | 49659 |
| 777 | Activity | 169.L.Q | 49689 |
| 787 | Activity | 826.V.M | 49667 |
| 788 | Activity | 891.S.Q | 49668 |
| 818 | Specificity | 698.S.R | 272 |

*Positions of mutations are shown relative to a CasX 515 sequence with an N-terminal methionine residue (SEQ ID NO: 49699). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

TABLE 20
CasX 515 domain sequences and coordinates

| | SEQ | | |
|---|---|---|---|
| Domain | ID NO | Amino Acid Sequence | Coordinates |
| N-terminal | — | M | 1 |
| methionine | | | |
| OBD-I | 295 | QEIKRINKIRRRLVKDSNTKKAGKTGPMKTLLVRVMTPDLRERLEN | 2-57 |
| | | LRKKPENIPQ | |
| Helical I-I | 296 | PISNTSRANLNKLLTDYTEMKKAILHVYWEEFQKDPVGLMSRVA | 58-101 |
| NTSB | 297 | QPASKKIDQNKLKPEMDEKGNLTTAGFACSQCGQPLFVYKLEQVSE | 102-192 |
| | | KGKAYTNYFGRCNVAEHEKLILLAQLKPEKDSDEAVTYSLGKFGQ | |
| Helical I-II | 298 | RALDFYSIHVTKESTHPVKPLAQIAGNRYASGPVGKALSDACMGTI | 193-333 |
| | | ASFLSKYQDIIIEHQKVVKGNQKRLESLRELAGKENLEYPSVTLPP | |
| | | QPHTKEGVDAYNEVIARVRMWVNLNLWQKLKLSRDDAKPLLRLKGF | |
| | | PSF | |
| Helical II | 299 | PLVERQANEVDWWDMVCNVKKLINEKKEDGKVEWQNLAGYKRQEAL | 334-501 |
| | | RPYLSSEEDRKKGKKFARYQLGDLLLHLEKKHGEDWGKVYDEAWER | |
| | | IDKKVEGLSKHIKLEEERRSEDAQSKAALTDWLRAKASFVIEGLKE | |
| | | ADKDEFCRCELKLQKWYGDLRGKPFAIEAE | |
| OBD-II | 300 | NSILDISGFSKQYNCAFIWQKDGVKKLNLYLIINYFKGGKLRFKKI | 502-647 |
| | | KPEAFEANRFYTVINKKSGEIVPMEVNENFDDPNLIILPLAFGKRQ | |
| | | GREFIWNDLLSLETGSLKLANGRVIEKTLYNRRTRQDEPALFVALT | |
| | | FERREVLD | |
| RuvC-I | 301 | SSNIKPMNLIGVDRGENIPAVIALTDPEGCPLSRFKDSLGNPTHIL | 648-811 |
| | | RIGESYKEKQRTIQAKKEVEQRRAGGYSRKYASKAKNLADDMVRNT | |
| | | ARDLLYYAVTQDAMLIFENLSRGFGRQGKRTEMAERQYTRMEDWLT | |
| | | AKLAYEGLPSKTYLSKTLAQYTSKTC | |
| TSL | 302 | SNCGFTITSADYDRVLEKLKKTATGWMTTINGKELKVEGQITYYNR | 812-921 |
| | | YKRQNVVKDLSVELDRLSEESVNNDISSWTKGRSGEALSLLKKRES | |
| | | HRPVQEKFVCLNCGFETH | |
| RuvC-II | 303 | ADEQAALNIARSWLFLRSQEYKKYQTNKTTGNTDKRAFVETWQSFY | 922-979 |
| | | RKKLKEVWKPAV | |

TABLE 21
Summary of positions of single mutations within CasX 515 protein domains

| | | Position of | | |
|---|---|---|---|---|
| | | mutation within | | Amino acid |
| | | CasX 515 domain* | | sequence of |
| CasX | | (Position. | | mutated |
| protein | Mutated | Reference. | Amino acid sequence of | domain |
| no. | domain | Alternative) | mutated domain† | (SEQ ID NO) |
| 532 | OBD-I | 26.—.R | QEIKRINKIRRRLVKDSNTKKAGKTRGPMK | 49800 |
| | | | TLLVRVMTPDLRERLENLRKKPENIPQ | |
| 536 | Helical I-II | 32.G.T | RALDFYSIHVTKESTHPVKPLAQIAGNRYA | 49801 |
| | | | STPVGKALSDACMGTIASELSKYQDIIIEH | |
| | | | QKVVKGNQKRLESLRELAGKENLEYPSVTL | |
| | | | PPQPHTKEGVDAYNEVIARVRMWVNLNLWQ | |
| | | | KLKLSRDDAKPLLRLKGFPSF | |
| 555 | NTSB | 70.A.D | QPASKKIDQNKLKPEMDEKGNLTTAGFACS | 49802 |
| | | | QCGQPLFVYKLEQVSEKGKAYTNYFGRCNV | |
| | | | AEHEKLILLDQLKPEKDSDEAVTYSLGKFG | |
| | | | Q | |
| 559 | OBD-I | 3.I.G | QEGKRINKIRRRLVKDSNTKKAGKTGPMKT | 49803 |
| | | | LLVRVMTPDLRERLENLRKKPENIPQ | |
| 561 | OBD-I | 4.—.G | QEIGKRINKIRRRLVKDSNTKKAGKTGPMK | 49804 |
| | | | TLLVRVMTPDLRERLENLRKKPENIPQ | |
| 562 | OBD-I | 4.K.G | QEIGRINKIRRRLVKDSNTKKAGKTGPMKT | 49805 |
| | | | LLVRVMTPDLRERLENLRKKPENIPQ | |
| 564 | OBD-I | 5.—.G | QEIKGRINKIRRRLVKDSNTKKAGKTGPMK | 49806 |
| | | | TLLVRVMTPDLRERLENLRKKPENIPQ | |
| 569 | OBD-I | 8.K.G | QEIKRINGIRRRLVKDSNTKKAGKTGPMKT | 49807 |
| | | | LLVRVMTPDLRERLENLRKKPENIPQ | |
| 572 | OBD-I | 34.R.P | QEIKRINKIRRRLVKDSNTKKAGKTGPMKT | 49808 |
| | | | LLVPVMTPDLRERLENLRKKPENIPQ | |
| 577 | Helical I-I | 7.R.Q | PISNTSQANLNKLLTDYTEMKKAILHVYWE | 49809 |
| | | | EFQKDPVGLMSRVA | |
| 583 | NTSB | 68.L.K | QPASKKIDQNKLKPEMDEKGNLTTAGFACS | 49810 |
| | | | QCGQPLFVYKLEQVSEKGKAYTNYFGRCNV | |
| | | | AEHEKLIKLAQLKPEKDSDEAVTYSLGKFG | |
| | | | Q | |
| 584 | NTSB | 70.A.Y | QPASKKIDQNKLKPEMDEKGNLTTAGFACS | 49811 |
| | | | QCGQPLFVYKLEQVSEKGKAYTNYFGRCNV | |
| | | | AEHEKLILLYQLKPEKDSDEAVTYSLGKFG | |
| | | | Q | |
| 585 | NTSB | 70.A.S | QPASKKIDQNKLKPEMDEKGNLTTAGFACS | 49812 |
| | | | QCGQPLFVYKLEQVSEKGKAYTNYFGRCNV | |
| | | | AEHEKLILLSQLKPEKDSDEAVTYSLGKFG | |
| | | | Q | |
| 592 | Helical I-II | 112.M.T | RALDFYSIHVTKESTHPVKPLAQIAGNRYA | 49813 |
| | | | SGPVGKALSDACMGTIASFLSKYQDIIIEH | |
| | | | QKVVKGNQKRLESLRELAGKENLEYPSVTL | |
| | | | PPQPHTKEGVDAYNEVIARVRTWVNLNLWQ | |
| | | | KLKLSRDDAKPLLRLKGFPSF | |
| 593 | Helical I-II | 112.M.W | RALDFYSIHVTKESTHPVKPLAQIAGNRYA | 49814 |
| | | | SGPVGKALSDACMGTIASFLSKYQDIIIEH | |
| | | | QKVVKGNQKRLESLRELAGKENLEYPSVTL | |
| | | | PPQPHTKEGVDAYNEVIARVRWWVNLNLWQ | |
| | | | KLKLSRDDAKPLLRLKGFPSF | |
| 607 | Helical II | 65.Y.T | PLVERQANEVDWWDMVCNVKKLINEKKEDG | 49815 |
| | | | KVFWQNLAGYKRQEALRPYLSSEEDRKKGK | |
| | | | KFARTQLGDLLLHLEKKHGEDWGKVYDEAW | |
| | | | ERIDKKVEGLSKHIKLEEERRSEDAQSKAA | |
| | | | LTDWLRAKASFVIEGLKEADKDEFCRCELK | |
| | | | LQKWYGDLRGKPFAIEAE | |
| 638 | Helical II | 148.E.D | PLVERQANEVDWWDMVCNVKKLINEKKEDG | 49816 |
| | | | KVEWQNLAGYKRQEALRPYLSSEEDRKKGK | |
| | | | KFARYQLGDLLLHLEKKHGEDWGKVYDEAW | |
| | | | ERIDKKVEGLSKHIKLEEERRSEDAQSKAA | |
| | | | LTDWLRAKASFVIEGLKEADKDEFCRCDLK | |
| | | | LQKWYGDLRGKPFAIEAE | |
| 656 | TSL | 76.T.D | SNCGFTITSADYDRVLEKLKKTATGWMTTI | 49817 |
| | | | NGKELKVEGQITYYNRYKRQNVVKDLSVEL | |
| | | | DRLSEESVNNDISSWDKGRSGEALSLLKKR | |
| | | | FSHRPVQEKFVCLNCGFETH | |
| 777 | NTSB | 68.L.Q | QPASKKIDQNKLKPEMDEKGNLTTAGFACS | 49818 |
| | | | QCGQPLFVYKLEQVSEKGKAYTNYFGRCNV | |
| | | | AEHEKLIQLAQLKPEKDSDEAVTYSLGKFG | |
| | | | Q | |
| 787 | TSL | 15.V.M | SNCGFTITSADYDRMLEKLKKTATGWMTTI | 49819 |
| | | | NGKELKVEGQITYYNRYKRQNVVKDLSVEL | |
| | | | DRLSEESVNNDISSWTKGRSGEALSLLKKR | |
| | | | FSHRPVQEKFVCLNCGFETH | |
| 788 | TSL | 80.S.Q | SNCGFTITSADYDRVLEKLKKTATGWMTTI | 49820 |
| | | | NGKELKVEGQITYYNRYKRQNVVKDLSVEL | |
| | | | DRLSEESVNNDISSWTKGRQGEALSLLKKR | |
| | | | FSHRPVQEKFVCLNCGFETH | |
| 818 | RuvC-I | 51.S.R | SSNIKPMNLIGVDRGENIPAVIALTDPEGC | 49821 |
| | | | PLSRFKDSLGNPTHILRIGERYKEKQRTIQ | |
| | | | AKKEVEQRRAGGYSRKYASKAKNLADDMVR | |
| | | | NTARDLLYYAVTQDAMLIFENLSRGFGRQG | |
| | | | KRTEMAERQYTRMEDWLTAKLAYEGLPSKT | |
| | | | YLSKTLAQYTSKTC | |

*Positions of mutations within domains are shown relative to the CasX 515 domain sequences provided in Table 20, above. Each mutation is indicated by its position, reference sequence, and alt sequence, separated by ‘.’ Insertions are indicated with a ‘—’ in the reference sequence (first position), and deletions with a ‘—’ in the alt sequence (second position).
†Mutated residues are bolded and underlined.

### Example 7: Engineered CasX Proteins with Pairs of Mutations Relative to CasX 515

Engineered CasX proteins were generated with pairs of mutations relative to CasX 515, and assessed for their on and off-target gene editing activity.

### Materials and Methods

Pairs of mutations listed in Tables 19 and 21, above, were introduced into the CasX 515 amino acid sequence to generate 161 amino acid sequences of engineered CasX proteins. The pairs of mutations and full-length amino acid sequences of the 161 engineered CasX proteins tested are listed in Tables 22, and Table 23 provides the amino acid sequences of each of the domains of the 161 engineered CasX proteins.

TABLE 22
Pairs of mutations and amino acid sequences
of engineered CasX proteins

| Mutations relative | | |
|---|---|---|
| to CasX 515* | | Full-length |
| (Position. | Phenotypes of | CasX amino |
| Reference. | single mutations | acid |
| Alternative) | (see Example 6) | SEQ ID NO |
| 4.I.G & 64.R.Q | Activity + activity | 28006 |
| 4.I.G & 169.L.K | Activity + activity | 28008 |
| 4.I.G & 169.L.Q | Activity + activity | 29119 |
| 4.I.G & 171.A.D | Activity + activity | 27952 |
| 4.I.G & 171.A.Y | Activity + activity | 28009 |
| 4.I.G & 171.A.S | Activity + activity | 28010 |
| 4.I.G & 224.G.T | Activity + activity | 31244 |
| 4.I.G & 304.M.T | Activity + activity | 28014 |
| 4.I.G & 398.Y.T | Activity + activity | 28018 |
| 4.I.G & 826.V.M | Activity + activity | 28035 |
| 4.I.G & 887.T.D | Activity + activity | 28027 |
| 4.I.G & 891.S.Q | Activity + activity | 28036 |
| 5.—.G & 64.R.Q | Activity + activity | 28050 |
| 5.—.G & 169.L.K | Activity + activity | 28052 |
| 5.—.G & 169.L.Q | Activity + activity | 29140 |
| 5.—.G & 171.A.D | Activity + activity | 27953 |
| 5.—.G & 171.A.Y | Activity + activity | 28053 |
| 5.—.G & 171.A.S | Activity + activity | 28054 |
| 5.—.G & 224.G.T | Activity + activity | 31592 |
| 5.—.G & 304.M.T | Activity + activity | 28058 |
| 5.—.G & 398.Y.T | Activity + activity | 28062 |
| 5.—.G & 826.V.M | Activity + activity | 28079 |
| 5.—.G & 887.T.D | Activity + activity | 28071 |
| 5.—.G & 891.S.Q | Activity + activity | 28080 |
| 9.K.G & 64.R.Q | Activity + activity | 28255 |
| 9.K.G & 169.L.K | Activity + activity | 28257 |
| 9.K.G & 169.L.Q | Activity + activity | 29245 |
| 9.K.G & 171.A.D | Activity + activity | 27958 |
| 9.K.G & 171.A.Y | Activity + activity | 28258 |
| 9.K.G & 171.A.S | Activity + activity | 28259 |
| 9.K.G & 224.G.T | Activity + activity | 33212 |
| 9.K.G & 304.M.T | Activity + activity | 28263 |
| 9.K.G & 398.Y.T | Activity + activity | 28267 |
| 9.K.G & 826.V.M | Activity + activity | 28284 |
| 9.K.G & 887.T.D | Activity + activity | 28276 |
| 9.K.G & 891.S.Q | Activity + activity | 28285 |
| 27.—.R & 64.R.Q | Activity + activity | 27868 |
| 27.—.R & 169.L.K | Activity + activity | 27870 |
| 27.—.R & 169.L.Q | Activity + activity | 29056 |
| 27.—.R & 171.A.D | Activity + activity | 27858 |
| 27.—.R & 171.A.Y | Activity + activity | 27871 |
| 27.—.R & 171.A.S | Activity + activity | 27872 |
| 27.—.R & 224.G.T | Activity + activity | 30196 |
| 27.—.R & 304.M.T | Activity + activity | 27876 |
| 27.—.R & 398.Y.T | Activity + activity | 27880 |
| 27.—.R & 826.V.M | Activity + activity | 27897 |
| 27.—.R & 887.T.D | Activity + activity | 27889 |
| 27.—.R & 891.S.Q | Activity + activity | 27898 |
| 35.R.P & 64.R.Q | Activity + activity | 28293 |
| 35.R.P & 169.L.K | Activity + activity | 28295 |
| 35.R.P & 169.L.Q | Activity + activity | 29266 |
| 35.R.P & 171.A.D | Activity + activity | 27959 |
| 35.R.P & 171.A.Y | Activity + activity | 28296 |
| 35.R.P & 171.A.S | Activity + activity | 28297 |
| 35.R.P & 224.G.T | Activity + activity | 33512 |
| 35.R.P & 304.M.T | Activity + activity | 28301 |
| 35.R.P & 398.Y.T | Activity + activity | 28305 |
| 35.R.P & 826.V.M | Activity + activity | 28322 |
| 35.R.P & 887.T.D | Activity + activity | 28314 |
| 35.R.P & 891.S.Q | Activity + activity | 28323 |
| 887.T.D & 891.S.Q | Activity + activity | 28926 |
| 64.R.Q & 169.L.K | Activity + activity | 28368 |
| 64.R.Q & 169.L.Q | Activity + activity | 29308 |
| 64.R.Q & 171.A.D | Activity + activity | 27961 |
| 64.R.Q & 171.A.Y | Activity + activity | 28369 |
| 64.R.Q & 171.A.S | Activity + activity | 28370 |
| 64.R.Q & 224.G.T | Activity + activity | 34088 |
| 64.R.Q & 304.M.T | Activity + activity | 28374 |
| 64.R.Q & 398.Y.T | Activity + activity | 28378 |
| 64.R.Q & 826.V.M | Activity + activity | 28395 |
| 64.R.Q & 887.T.D | Activity + activity | 28387 |
| 64.R.Q & 891.S.Q | Activity + activity | 28396 |
| 169.L.K & 171.A.D | Activity + activity | 27963 |
| 169.L.K & 171.A.Y | Activity + activity | 28438 |
| 169.L.K & 171.A.S | Activity + activity | 28439 |
| 169.L.K & 224.G.T | Activity + activity | 34631 |
| 169.L.K & 304.M.T | Activity + activity | 28443 |
| 169.L.K & 398.Y.T | Activity + activity | 28447 |
| 169.L.K & 826.V.M | Activity + activity | 28464 |
| 169.L.K & 887.T.D | Activity + activity | 28456 |
| 169.L.K & 891.S.Q | Activity + activity | 28465 |
| 169.L.Q & 171.A.D | Activity + activity | 29098 |
| 169.L.Q & 171.A.Y | Activity + activity | 29371 |
| 169.L.Q & 171.A.S | Activity + activity | 29392 |
| 169.L.Q & 224.G.T | Activity + activity | 43373 |
| 169.L.Q & 304.M.T | Activity + activity | 29476 |
| 169.L.Q & 398.Y.T | Activity + activity | 29560 |
| 169.L.Q & 826.V.M | Activity + activity | 29917 |
| 169.L.Q & 887.T.D | Activity + activity | 29749 |
| 169.L.Q & 891.S.Q | Activity + activity | 29938 |
| 171.A.D & 224.G.T | Activity + activity | 30888 |
| 171.A.D & 304.M.T | Activity + activity | 27969 |
| 171.A.D & 398.Y.T | Activity + activity | 27973 |
| 171.A.D & 826.V.M | Activity + activity | 27990 |
| 171.A.D & 887.T.D | Activity + activity | 27982 |
| 171.A.D & 891.S.Q | Activity + activity | 27991 |
| 171.A.Y & 224.G.T | Activity + activity | 34870 |
| 171.A.Y & 304.M.T | Activity + activity | 28477 |
| 171.A.Y & 398.Y.T | Activity + activity | 28481 |
| 171.A.Y & 826.V.M | Activity + activity | 28498 |
| 171.A.Y & 887.T.D | Activity + activity | 28490 |
| 171.A.Y & 891.S.Q | Activity + activity | 28499 |
| 171.A.S & 224.G.T | Activity + activity | 35139 |
| 171.A.S & 304.M.T | Activity + activity | 28511 |
| 171.A.S & 398.Y.T | Activity + activity | 28515 |
| 171.A.S & 826.V.M | Activity + activity | 28532 |
| 171.A.S & 887.T.D | Activity + activity | 28524 |
| 171.A.S & 891.S.Q | Activity + activity | 28533 |
| 4.I.G & 35.R.P | Activity + activity | 28004 |
| 224.G.T & 304.M.T | Activity + activity | 35402 |
| 224.G.T & 398.Y.T | Activity + activity | 35422 |
| 224.G.T & 826.V.M | Activity + activity | 35507 |
| 224.G.T & 887.T.D | Activity + activity | 35467 |
| 224.G.T & 891.S.Q | Activity + activity | 35512 |
| 5.—.G & 35.R.P | Activity + activity | 28048 |
| 4.I.G & 27.—.R | Activity + activity | 27859 |
| 304.M.T & 398.Y.T | Activity + activity | 28633 |
| 304.M.T & 826.V.M | Activity + activity | 28650 |
| 304.M.T & 887.T.D | Activity + activity | 28642 |
| 304.M.T & 891.S.Q | Activity + activity | 28651 |
| 9.K.G & 35.R.P | Activity + activity | 28253 |
| 5.—.G & 27.—.R | Activity + activity | 49746 |
| 4.I.G & 9.K.G | Activity + activity | 28003 |
| 398.Y.T & 826.V.M | Activity + activity | 28753 |
| 398.Y.T & 887.T.D | Activity + activity | 28745 |
| 398.Y.T & 891.S.Q | Activity + activity | 28754 |
| 27.—.R & 35.R.P | Activity + activity | 27866 |
| 9.K.G & 27.—.R | Activity + activity | 27865 |
| 5.—.G & 9.K.G | Activity + activity | 28047 |
| 4.I.G & 5.—.G | Activity + activity | 27998 |
| 826.V.M & 887.T.D | Activity + activity | 28925 |
| 826.V.M & 891.S.Q | Activity + activity | 29011 |
| 5.K.G & 27.—.R | Activity + specificity | 27861 |
| 5.K.G & 169.L.K | Activity + specificity | 28095 |
| 5.K.G & 171.A.D | Activity + specificity | 27954 |
| 5.K.G & 304.M.T | Activity + specificity | 28101 |
| 5.K.G & 398.Y.T | Activity + specificity | 28105 |
| 5.K.G & 891.S.Q | Activity + specificity | 28123 |
| 6.—.G & 27.—.R | Activity + specificity | 49747 |
| 6.—.G & 169.L.K | Activity + specificity | 28137 |
| 6.—.G & 171.A.D | Activity + specificity | 27955 |
| 6.—.G & 304.M.T | Activity + specificity | 28143 |
| 6.—.G & 398.Y.T | Activity + specificity | 28147 |
| 6.—.G & 891.S.Q | Activity + specificity | 28165 |
| 304.M.W & 27.—.R | Activity + specificity | 27877 |
| 304.M.W & 169.L.K | Activity + specificity | 28444 |
| 304.M.W & 171.A.D | Activity + specificity | 27970 |
| 304.M.W & 398.Y.T | Activity + specificity | 28661 |
| 304.M.W & 891.S.Q | Activity + specificity | 28679 |
| 481.E.D & 27.—.R | Activity + specificity | 27882 |
| 481.E.D & 169.L.K | Activity + specificity | 28449 |
| 481.E.D & 171.A.D | Activity + specificity | 27975 |
| 481.E.D & 304.M.T | Activity + specificity | 28635 |
| 481.E.D & 398.Y.T | Activity + specificity | 28738 |
| 481.E.D & 891.S.Q | Activity + specificity | 28799 |
| 698.S.R & 27.—.R | Activity + specificity | 27903 |
| 698.S.R & 169.L.K | Activity + specificity | 28470 |
| 698.S.R & 171.A.D | Activity + specificity | 27996 |
| 698.S.R & 304.M.T | Activity + specificity | 28656 |
| 698.S.R & 398.Y.T | Activity + specificity | 28759 |
| 698.S.R & 891.S.Q | Activity + specificity | 29022 |

*Positions of mutations are shown relative to a CasX 515 sequence with an N-terminal methionine residue (SEQ ID NO: 49699). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

TABLE 23
Amino acid sequences of domains of engineered CasX proteins, N- to C-terminus

| Mutations relative | | | | | | | | | |
|---|---|---|---|---|---|---|---|---|---|
| to CasX 515* | | | | | | | | | |
| (Position. Reference. | Amino acid sequences of domains of engineered CasX proteins (SEQ ID NOs) | | | | | | | | |
| Alternative) | OBD-I | Helical I-I | NTSB | Helical I-II | Helical II | OBD-II | RuvC-I | TSL | RuvC-II |
| None (non-mutated | 295 | 296 | 297 | 298 | 299 | 300 | 301 | 302 | 303 |
| CasX 515) | | | | | | | | | |
| 4.I.G & 64.R.Q | 49803 | 49809 | 297 | 298 | 299 | 300 | 301 | 302 | 303 |
| 4.I.G & 169.L.K | 49803 | 296 | 49810 | 298 | 299 | 300 | 301 | 302 | 303 |
| 4.I.G & 169.L.Q | 49803 | 296 | 49818 | 298 | 299 | 300 | 301 | 302 | 303 |
| 4.I.G & 171.A.D | 49803 | 296 | 49802 | 298 | 299 | 300 | 301 | 302 | 303 |
| 4.I.G & 171.A.Y | 49803 | 296 | 49811 | 298 | 299 | 300 | 301 | 302 | 303 |
| 4.I.G & 171.A.S | 49803 | 296 | 49812 | 298 | 299 | 300 | 301 | 302 | 303 |
| 4.I.G & 224.G.T | 49803 | 296 | 297 | 49801 | 299 | 300 | 301 | 302 | 303 |
| 4.I.G & 304.M.T | 49803 | 296 | 297 | 49813 | 299 | 300 | 301 | 302 | 303 |
| 4.I.G & 398.Y.T | 49803 | 296 | 297 | 298 | 49815 | 300 | 301 | 302 | 303 |
| 4.I.G & 826.V.M | 49803 | 296 | 297 | 298 | 299 | 300 | 301 | 49819 | 303 |
| 4.I.G & 887.T.D | 49803 | 296 | 297 | 298 | 299 | 300 | 301 | 49817 | 303 |
| 4.I.G & 891.S.Q | 49803 | 296 | 297 | 298 | 299 | 300 | 301 | 49820 | 303 |
| 5.—.G & 64.R.Q | 49804 | 49809 | 297 | 298 | 299 | 300 | 301 | 302 | 303 |
| 5.—.G & 169.L.K | 49804 | 296 | 49810 | 298 | 299 | 300 | 301 | 302 | 303 |
| 5.—.G & 169.L.Q | 49804 | 296 | 49818 | 298 | 299 | 300 | 301 | 302 | 303 |
| 5.—.G & 171.A.D | 49804 | 296 | 49802 | 298 | 299 | 300 | 301 | 302 | 303 |
| 5.—.G & 171.A.Y | 49804 | 296 | 49811 | 298 | 299 | 300 | 301 | 302 | 303 |
| 5.—.G & 171.A.S | 49804 | 296 | 49812 | 298 | 299 | 300 | 301 | 302 | 303 |
| 5.—.G & 224.G.T | 49804 | 296 | 297 | 49801 | 299 | 300 | 301 | 302 | 303 |
| 5.—.G & 304.M.T | 49804 | 296 | 297 | 49813 | 299 | 300 | 301 | 302 | 303 |
| 5.—.G & 398.Y.T | 49804 | 296 | 297 | 298 | 49815 | 300 | 301 | 302 | 303 |
| 5.—.G & 826.V.M | 49804 | 296 | 297 | 298 | 299 | 300 | 301 | 49819 | 303 |
| 5.—.G & 887.T.D | 49804 | 296 | 297 | 298 | 299 | 300 | 301 | 49817 | 303 |
| 5.—.G & 891.S.Q | 49804 | 296 | 297 | 298 | 299 | 300 | 301 | 49820 | 303 |
| 9.K.G & 64.R.Q | 49807 | 49809 | 297 | 298 | 299 | 300 | 301 | 302 | 303 |
| 9.K.G & 169.L.K | 49807 | 296 | 49810 | 298 | 299 | 300 | 301 | 302 | 303 |
| 9.K.G & 169.L.Q | 49807 | 296 | 49818 | 298 | 299 | 300 | 301 | 302 | 303 |
| 9.K.G & 171.A.D | 49807 | 296 | 49802 | 298 | 299 | 300 | 301 | 302 | 303 |
| 9.K.G & 171.A.Y | 49807 | 296 | 49811 | 298 | 299 | 300 | 301 | 302 | 303 |
| 9.K.G & 171.A.S | 49807 | 296 | 49812 | 298 | 299 | 300 | 301 | 302 | 303 |
| 9.K.G & 224.G.T | 49807 | 296 | 297 | 49801 | 299 | 300 | 301 | 302 | 303 |
| 9.K.G & 304.M.T | 49807 | 296 | 297 | 49813 | 299 | 300 | 301 | 302 | 303 |
| 9.K.G & 398.Y.T | 49807 | 296 | 297 | 298 | 49815 | 300 | 301 | 302 | 303 |
| 9.K.G & 826.V.M | 49807 | 296 | 297 | 298 | 299 | 300 | 301 | 49819 | 303 |
| 9.K.G & 887.T.D | 49807 | 296 | 297 | 298 | 299 | 300 | 301 | 49817 | 303 |
| 9.K.G & 891.S.Q | 49807 | 296 | 297 | 298 | 299 | 300 | 301 | 49820 | 303 |
| 27.—.R & 64.R.Q | 49800 | 49809 | 297 | 298 | 299 | 300 | 301 | 302 | 303 |
| 27.—.R & 169.L.K | 49800 | 296 | 49810 | 298 | 299 | 300 | 301 | 302 | 303 |
| 27.—.R & 169.L.Q | 49800 | 296 | 49818 | 298 | 299 | 300 | 301 | 302 | 303 |
| 27.—.R & 171.A.D | 49800 | 296 | 49802 | 298 | 299 | 300 | 301 | 302 | 303 |
| 27.—.R & 171.A.Y | 49800 | 296 | 49811 | 298 | 299 | 300 | 301 | 302 | 303 |
| 27.—.R & 171.A.S | 49800 | 296 | 49812 | 298 | 299 | 300 | 301 | 302 | 303 |
| 27.—.R & 224.G.T | 49800 | 296 | 297 | 49801 | 299 | 300 | 301 | 302 | 303 |
| 27.—.R & 304.M.T | 49800 | 296 | 297 | 49813 | 299 | 300 | 301 | 302 | 303 |
| 27.—.R & 398.Y.T | 49800 | 296 | 297 | 298 | 49815 | 300 | 301 | 302 | 303 |
| 27.—.R & 826.V.M | 49800 | 296 | 297 | 298 | 299 | 300 | 301 | 49819 | 303 |
| 27.—.R & 887.T.D | 49800 | 296 | 297 | 298 | 299 | 300 | 301 | 49817 | 303 |
| 27.—.R & 891.S.Q | 49800 | 296 | 297 | 298 | 299 | 300 | 301 | 49820 | 303 |
| 35.R.P & 64.R.Q | 49808 | 49809 | 297 | 298 | 299 | 300 | 301 | 302 | 303 |
| 35.R.P & 169.L.K | 49808 | 296 | 49810 | 298 | 299 | 300 | 301 | 302 | 303 |
| 35.R.P & 169.L.Q | 49808 | 296 | 49818 | 298 | 299 | 300 | 301 | 302 | 303 |
| 35.R.P & 171.A.D | 49808 | 296 | 49802 | 298 | 299 | 300 | 301 | 302 | 303 |
| 35.R.P & 171.A.Y | 49808 | 296 | 49811 | 298 | 299 | 300 | 301 | 302 | 303 |
| 35.R.P & 171.A.S | 49808 | 296 | 49812 | 298 | 299 | 300 | 301 | 302 | 303 |
| 35.R.P & 224.G.T | 49808 | 296 | 297 | 49801 | 299 | 300 | 301 | 302 | 303 |
| 35.R.P & 304.M.T | 49808 | 296 | 297 | 49813 | 299 | 300 | 301 | 302 | 303 |
| 35.R.P & 398.Y.T | 49808 | 296 | 297 | 298 | 49815 | 300 | 301 | 302 | 303 |
| 35.R.P & 826.V.M | 49808 | 296 | 297 | 298 | 299 | 300 | 301 | 49819 | 303 |
| 35.R.P & 887.T.D | 49808 | 296 | 297 | 298 | 299 | 300 | 301 | 49817 | 303 |
| 35.R.P & 891.S.Q | 49808 | 296 | 297 | 298 | 299 | 300 | 301 | 49820 | 303 |
| 887.T.D & 891.S.Q | 295 | 296 | 297 | 298 | 299 | 300 | 301 | 49844 | 303 |
| 64.R.Q & 169.L.K | 295 | 49809 | 49810 | 298 | 299 | 300 | 301 | 302 | 303 |
| 64.R.Q & 169.L.Q | 295 | 49809 | 49818 | 298 | 299 | 300 | 301 | 302 | 303 |
| 64.R.Q & 171.A.D | 295 | 49809 | 49802 | 298 | 299 | 300 | 301 | 302 | 303 |
| 64.R.Q & 171.A.Y | 295 | 49809 | 49811 | 298 | 299 | 300 | 301 | 302 | 303 |
| 64.R.Q & 171.A.S | 295 | 49809 | 49812 | 298 | 299 | 300 | 301 | 302 | 303 |
| 64.R.Q & 224.G.T | 295 | 49809 | 297 | 49801 | 299 | 300 | 301 | 302 | 303 |
| 64.R.Q & 304.M.T | 295 | 49809 | 297 | 49813 | 299 | 300 | 301 | 302 | 303 |
| 64.R.Q & 398.Y.T | 295 | 49809 | 297 | 298 | 49815 | 300 | 301 | 302 | 303 |
| 64.R.Q & 826.V.M | 295 | 49809 | 297 | 298 | 299 | 300 | 301 | 49819 | 303 |
| 64.R.Q & 887.T.D | 295 | 49809 | 297 | 298 | 299 | 300 | 301 | 49817 | 303 |
| 64.R.Q & 891.S.Q | 295 | 49809 | 297 | 298 | 299 | 300 | 301 | 49820 | 303 |
| 169.L.K & 171.A.D | 295 | 296 | 49835 | 298 | 299 | 300 | 301 | 302 | 303 |
| 169.L.K & 171.A.Y | 295 | 296 | 49836 | 298 | 299 | 300 | 301 | 302 | 303 |
| 169.L.K & 171.A.S | 295 | 296 | 49837 | 298 | 299 | 300 | 301 | 302 | 303 |
| 169.L.K & 224.G.T | 295 | 296 | 49810 | 49801 | 299 | 300 | 301 | 302 | 303 |
| 169.L.K & 304.M.T | 295 | 296 | 49810 | 49813 | 299 | 300 | 301 | 302 | 303 |
| 169.L.K & 398.Y.T | 295 | 296 | 49810 | 298 | 49815 | 300 | 301 | 302 | 303 |
| 169.L.K & 826.V.M | 295 | 296 | 49810 | 298 | 299 | 300 | 301 | 49819 | 303 |
| 169.L.K & 887.T.D | 295 | 296 | 49810 | 298 | 299 | 300 | 301 | 49817 | 303 |
| 169.L.K & 891.S.Q | 295 | 296 | 49810 | 298 | 299 | 300 | 301 | 49820 | 303 |
| 169.L.Q & 171.A.D | 295 | 296 | 49838 | 298 | 299 | 300 | 301 | 302 | 303 |
| 169.L.Q & 171.A.Y | 295 | 296 | 49839 | 298 | 299 | 300 | 301 | 302 | 303 |
| 169.L.Q & 171.A.S | 295 | 296 | 49840 | 298 | 299 | 300 | 301 | 302 | 303 |
| 169.L.Q & 224.G.T | 295 | 296 | 49818 | 49801 | 299 | 300 | 301 | 302 | 303 |
| 169.L.Q & 304.M.T | 295 | 296 | 49818 | 49813 | 299 | 300 | 301 | 302 | 303 |
| 169.L.Q & 398.Y.T | 295 | 296 | 49818 | 298 | 49815 | 300 | 301 | 302 | 303 |
| 169.L.Q & 826.V.M | 295 | 296 | 49818 | 298 | 299 | 300 | 301 | 49819 | 303 |
| 169.L.Q & 887.T.D | 295 | 296 | 49818 | 298 | 299 | 300 | 301 | 49817 | 303 |
| 169.L.Q & 891.S.Q | 295 | 296 | 49818 | 298 | 299 | 300 | 301 | 49820 | 303 |
| 171.A.D & 224.G.T | 295 | 296 | 49802 | 49801 | 299 | 300 | 301 | 302 | 303 |
| 171.A.D & 304.M.T | 295 | 296 | 49802 | 49813 | 299 | 300 | 301 | 302 | 303 |
| 171.A.D & 398.Y.T | 295 | 296 | 49802 | 298 | 49815 | 300 | 301 | 302 | 303 |
| 171.A.D & 826.V.M | 295 | 296 | 49802 | 298 | 299 | 300 | 301 | 49819 | 303 |
| 171.A.D & 887.T.D | 295 | 296 | 49802 | 298 | 299 | 300 | 301 | 49817 | 303 |
| 171.A.D & 891.S.Q | 295 | 296 | 49802 | 298 | 299 | 300 | 301 | 49820 | 303 |
| 171.A.Y & 224.G.T | 295 | 296 | 49811 | 49801 | 299 | 300 | 301 | 302 | 303 |
| 171.A.Y & 304.M.T | 295 | 296 | 49811 | 49813 | 299 | 300 | 301 | 302 | 303 |
| 171.A.Y & 398.Y.T | 295 | 296 | 49811 | 298 | 49815 | 300 | 301 | 302 | 303 |
| 171.A.Y & 826.V.M | 295 | 296 | 49811 | 298 | 299 | 300 | 301 | 49819 | 303 |
| 171.A.Y & 887.T.D | 295 | 296 | 49811 | 298 | 299 | 300 | 301 | 49817 | 303 |
| 171.A.Y & 891.S.Q | 295 | 296 | 49811 | 298 | 299 | 300 | 301 | 49820 | 303 |
| 171.A.S & 224.G.T | 295 | 296 | 49812 | 49801 | 299 | 300 | 301 | 302 | 303 |
| 171.A.S & 304.M.T | 295 | 296 | 49812 | 49813 | 299 | 300 | 301 | 302 | 303 |
| 171.A.S & 398.Y.T | 295 | 296 | 49812 | 298 | 49815 | 300 | 301 | 302 | 303 |
| 171.A.S & 826.V.M | 295 | 296 | 49812 | 298 | 299 | 300 | 301 | 49819 | 303 |
| 171.A.S & 887.T.D | 295 | 296 | 49812 | 298 | 299 | 300 | 301 | 49817 | 303 |
| 171.A.S & 891.S.Q | 295 | 296 | 49812 | 298 | 299 | 300 | 301 | 49820 | 303 |
| 4.I.G & 35.R.P | 49822 | 296 | 297 | 298 | 299 | 300 | 301 | 302 | 303 |
| 224.G.T & 304.M.T | 295 | 296 | 297 | 49842 | 299 | 300 | 301 | 302 | 303 |
| 224.G.T & 398.Y.T | 295 | 296 | 297 | 49801 | 49815 | 300 | 301 | 302 | 303 |
| 224.G.T & 826.V.M | 295 | 296 | 297 | 49801 | 299 | 300 | 301 | 49819 | 303 |
| 224.G.T & 887.T.D | 295 | 296 | 297 | 49801 | 299 | 300 | 301 | 49817 | 303 |
| 224.G.T & 891.S.Q | 295 | 296 | 297 | 49801 | 299 | 300 | 301 | 49820 | 303 |
| 5.—.G & 35.R.P | 49823 | 296 | 297 | 298 | 299 | 300 | 301 | 302 | 303 |
| 4.I.G & 27.—.R | 49824 | 296 | 297 | 298 | 299 | 300 | 301 | 302 | 303 |
| 304.M.T & 398.Y.T | 295 | 296 | 297 | 49813 | 49815 | 300 | 301 | 302 | 303 |
| 304.M.T & 826.V.M | 295 | 296 | 297 | 49813 | 299 | 300 | 301 | 49819 | 303 |
| 304.M.T & 887.T.D | 295 | 296 | 297 | 49813 | 299 | 300 | 301 | 49817 | 303 |
| 304.M.T & 891.S.Q | 295 | 296 | 297 | 49813 | 299 | 300 | 301 | 49820 | 303 |
| 9.K.G & 35.R.P | 49825 | 296 | 297 | 298 | 299 | 300 | 301 | 302 | 303 |
| 5.—.G & 27.—.R | 49826 | 296 | 297 | 298 | 299 | 300 | 301 | 302 | 303 |
| 4.I.G & 9.K.G | 49827 | 296 | 297 | 298 | 299 | 300 | 301 | 302 | 303 |
| 398.Y.T & 826.V.M | 295 | 296 | 297 | 298 | 49815 | 300 | 301 | 49819 | 303 |
| 398.Y.T & 887.T.D | 295 | 296 | 297 | 298 | 49815 | 300 | 301 | 49817 | 303 |
| 398.Y.T & 891.S.Q | 295 | 296 | 297 | 298 | 49815 | 300 | 301 | 49820 | 303 |
| 27.—.R & 35.R.P | 49828 | 296 | 297 | 298 | 299 | 300 | 301 | 302 | 303 |
| 9.K.G & 27.—.R | 49829 | 296 | 297 | 298 | 299 | 300 | 301 | 302 | 303 |
| 5.—.G & 9.K.G | 49830 | 296 | 297 | 298 | 299 | 300 | 301 | 302 | 303 |
| 4.I.G & 5.—.G | 49831 | 296 | 297 | 298 | 299 | 300 | 301 | 302 | 303 |
| 826.V.M & 887.T.D | 295 | 296 | 297 | 298 | 299 | 300 | 301 | 49845 | 303 |
| 826.V.M & 891.S.Q | 295 | 296 | 297 | 298 | 299 | 300 | 301 | 49846 | 303 |
| 5.K.G & 27.—.R | 49832 | 296 | 297 | 298 | 299 | 300 | 301 | 302 | 303 |
| 5.K.G & 169.L.K | 49805 | 296 | 49810 | 298 | 299 | 300 | 301 | 302 | 303 |
| 5.K.G & 171.A.D | 49805 | 296 | 49802 | 298 | 299 | 300 | 301 | 302 | 303 |
| 5.K.G & 304.M.T | 49805 | 296 | 297 | 49813 | 299 | 300 | 301 | 302 | 303 |
| 5.K.G & 398.Y.T | 49805 | 296 | 297 | 298 | 49815 | 300 | 301 | 302 | 303 |
| 5.K.G & 891.S.Q | 49805 | 296 | 297 | 298 | 299 | 300 | 301 | 49820 | 303 |
| 6.—.G & 27.—.R | 49833 | 296 | 297 | 298 | 299 | 300 | 301 | 302 | 303 |
| 6.—.G & 169.L.K | 49806 | 296 | 49810 | 298 | 299 | 300 | 301 | 302 | 303 |
| 6.—.G & 171.A.D | 49806 | 296 | 49802 | 298 | 299 | 300 | 301 | 302 | 303 |
| 6.—.G & 304.M.T | 49806 | 296 | 297 | 49813 | 299 | 300 | 301 | 302 | 303 |
| 6.—.G & 398.Y.T | 49806 | 296 | 297 | 298 | 49815 | 300 | 301 | 302 | 303 |
| 6.—.G & 891.S.Q | 49806 | 296 | 297 | 298 | 299 | 300 | 301 | 49820 | 303 |
| 304.M.W & 27.—.R | 49800 | 296 | 297 | 49814 | 299 | 300 | 301 | 302 | 303 |
| 304.M.W & 169.L.K | 295 | 296 | 49810 | 49814 | 299 | 300 | 301 | 302 | 303 |
| 304.M.W & 171.A.D | 295 | 296 | 49802 | 49814 | 299 | 300 | 301 | 302 | 303 |
| 304.M.W & 398.Y.T | 295 | 296 | 297 | 49814 | 49815 | 300 | 301 | 302 | 303 |
| 304.M.W & 891.S.Q | 295 | 296 | 297 | 49814 | 299 | 300 | 301 | 49820 | 303 |
| 481.E.D & 27.—.R | 49800 | 296 | 297 | 298 | 49816 | 300 | 301 | 302 | 303 |
| 481.E.D & 169.L.K | 295 | 296 | 49810 | 298 | 49816 | 300 | 301 | 302 | 303 |
| 481.E.D & 171.A.D | 295 | 296 | 49802 | 298 | 49816 | 300 | 301 | 302 | 303 |
| 481.E.D & 304.M.T | 295 | 296 | 297 | 49813 | 49816 | 300 | 301 | 302 | 303 |
| 481.E.D & 398.Y.T | 295 | 296 | 297 | 298 | 49843 | 300 | 301 | 302 | 303 |
| 481.E.D & 891.S.Q | 295 | 296 | 297 | 298 | 49816 | 300 | 301 | 49820 | 303 |
| 698.S.R & 27.—.R | 49800 | 296 | 297 | 298 | 299 | 300 | 49821 | 302 | 303 |
| 698.S.R & 169.L.K | 295 | 296 | 49810 | 298 | 299 | 300 | 49821 | 302 | 303 |
| 698.S.R & 171.A.D | 295 | 296 | 49802 | 298 | 299 | 300 | 49821 | 302 | 303 |
| 698.S.R & 304.M.T | 295 | 296 | 297 | 49813 | 299 | 300 | 49821 | 302 | 303 |
| 698.S.R & 398.Y.T | 295 | 296 | 297 | 298 | 49815 | 300 | 49821 | 302 | 303 |
| 698.S.R & 891.S.Q | 295 | 296 | 297 | 298 | 299 | 300 | 49821 | 49820 | 303 |

*Positions of mutations are shown relative to a CasX 515 sequence with an N-terminal methionine residue (SEQ ID NO: 49699). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

A subset of these 161 engineered CasX proteins were cloned using methods standard in the art, and are listed in Tables 25 and 27, below. In addition, an engineered CasX protein termed CasX 1001 was generated by combining mutations from engineered CasX protein 812 and CasX variant 676 (27.-.R, 169.L.K, and 329.G.K mutations relative to CasX 515), which have been previously validated as a highly specific and highly active CasX proteins, respectively (the PAM-altering 224.G.S mutation also present in CasX 676 was not included). Engineered CasX protein 969 was generated by combining 27.-.R, 171.A.D, and 224.G.T mutations relative to CasX 515. Finally, engineered CasX protein 973 was generated by combining 35.R.P, 171.A.Y, and 304.M.T mutations relative to CasX 515. The amino acid sequences of engineered CasX proteins 969, 973, and 1001 are provided in Table 24, below.

TABLE 24
Amino acid sequences of engineered CasX proteins 969, 973, and 1001

| CasX | | |
|---|---|---|
| protein | | SEQ |
| no. | Amino acid sequence | ID NO |
| 969 | QEIKRINKIRRRLVKDSNTKKAGKTRGPMKTLLVRVMTPDLRERLENLRKKPENIPQPISNTSR | 49871 |
| | ANLNKLLTDYTEMKKAILHVYWEEFQKDPVGLMSRVAQPASKKIDQNKLKPEMDEKGNLTTAGF | |
| | ACSQCGQPLFVYKLEQVSEKGKAYTNYFGRCNVAEHEKLILLDQLKPEKDSDEAVTYSLGKFGQ | |
| | RALDFYSIHVTKESTHPVKPLAQIAGNRYASTPVGKALSDACMGTIASFLSKYQDIIIEHQKVV | |
| | KGNQKRLESLRELAGKENLEYPSVTLPPQPHTKEGVDAYNEVIARVRMWVNLNLWQKLKLSRDD | |
| | AKPLLRLKGFPSFPLVERQANEVDWWDMVCNVKKLINEKKEDGKVEWQNLAGYKRQEALRPYLS | |
| | SEEDRKKGKKFARYQLGDLLLHLEKKHGEDWGKVYDEAWERIDKKVEGLSKHIKLEEERRSEDA | |
| | QSKAALTDWLRAKASFVIEGLKEADKDEFCRCELKLQKWYGDLRGKPFAIEAENSILDISGESK | |
| | QYNCAFIWQKDGVKKLNLYLIINYFKGGKLRFKKIKPEAFEANRFYTVINKKSGEIVPMEVNEN | |
| | FDDPNLIILPLAFGKRQGREFIWNDLLSLETGSLKLANGRVIEKTLYNRRTRQDEPALFVALTE | |
| | ERREVLDSSNIKPMNLIGVDRGENIPAVIALTDPEGCPLSRFKDSLGNPTHILRIGESYKEKQR | |
| | TIQAKKEVEQRRAGGYSRKYASKAKNLADDMVRNTARDLLYYAVTQDAMLIFENLSRGFGRQGK | |
| | RTFMAERQYTRMEDWLTAKLAYEGLPSKTYLSKTLAQYTSKTCSNCGFTITSADYDRVLEKLKK | |
| | TATGWMTTINGKELKVEGQITYYNRYKRQNVVKDLSVELDRLSEESVNNDISSWTKGRSGEALS | |
| | LLKKRFSHRPVQEKFVCLNCGFETHADEQAALNIARSWLFLRSQEYKKYQTNKTTGNTDKRAFV | |
| | ETWQSFYRKKLKEVWKPAV | |
| 973 | QEIKRINKIRRRLVKDSNTKKAGKTGPMKTLLVPVMTPDLRERLENLRKKPENIPQPISNTSRA | 49872 |
| | NLNKLLTDYTEMKKAILHVYWEEFQKDPVGLMSRVAQPASKKIDQNKLKPEMDEKGNLTTAGFA | |
| | CSQCGQPLFVYKLEQVSEKGKAYTNYFGRCNVAEHEKLILLYQLKPEKDSDEAVTYSLGKFGQR | |
| | ALDFYSIHVTKESTHPVKPLAQIAGNRYASGPVGKALSDACMGTIASFLSKYQDIIIEHQKVVK | |
| | GNQKRLESLRELAGKENLEYPSVTLPPQPHTKEGVDAYNEVIARVRTWVNLNLWQKLKLSRDDA | |
| | KPLLRLKGFPSFPLVERQANEVDWWDMVCNVKKLINEKKEDGKVFWQNLAGYKRQEALRPYLSS | |
| | EEDRKKGKKFARYQLGDLLLHLEKKHGEDWGKVYDEAWERIDKKVEGLSKHIKLEEERRSEDAQ | |
| | SKAALTDWLRAKASFVIEGLKEADKDEFCRCELKLQKWYGDLRGKPFAIEAENSILDISGFSKQ | |
| | YNCAFIWQKDGVKKLNLYLIINYFKGGKLRFKKIKPEAFEANRFYTVINKKSGEIVPMEVNENF | |
| | DDPNLIILPLAFGKRQGREFIWNDLLSLETGSLKLANGRVIEKTLYNRRTRQDEPALFVALTFE | |
| | RREVLDSSNIKPMNLIGVDRGENIPAVIALTDPEGCPLSRFKDSLGNPTHILRIGESYKEKQRT | |
| | IQAKKEVEQRRAGGYSRKYASKAKNLADDMVRNTARDLLYYAVTQDAMLIFENLSRGFGRQGKR | |
| | TEMAERQYTRMEDWLTAKLAYEGLPSKTYLSKTLAQYTSKTCSNCGFTITSADYDRVLEKLKKT | |
| | ATGWMTTINGKELKVEGQITYYNRYKRQNVVKDLSVELDRLSEESVNNDISSWTKGRSGEALSL | |
| | LKKRFSHRPVQEKFVCLNCGFETHADEQAALNIARSWLFLRSQEYKKYQTNKTTGNTDKRAFVE | |
| | TWQSFYRKKLKEVWKPAV | |
| 1001 | QEIKRINKIRRRLVKDSNTKKAGKTRGPMKTLLVRVMTPDLRERLENLRKKPENIPQPISNTSR | 49873 |
| | ANLNKLLTDYTEMKKAILHVYWEEFQKDPVGLMSRVAQPASKKIDQNKLKPEMDEKGNLTTAGF | |
| | ACSQCGQPLFVYKLEQVSEKGKAYTNYFGRCNVAEHEKLIKLAQLKPEKDSDEAVTYSLGKFGQ | |
| | RALDFYSIHVTKESTHPVKPLAQIAGNRYASGPVGKALSDACMGTIASFLSKYQDIIIEHQKVV | |
| | KGNQKRLESLRELAGKENLEYPSVTLPPQPHTKEGVDAYNEVIARVRMWVNLNLWQKLKLSRDD | |
| | AKPLLRLKKFPSFPLVERQANEVDWWDMVCNVKKLINEKKEDGKVEWQNLAGYKRQEALRPYLS | |
| | SEEDRKKGKKFARYQLGDLLLHLEKKHGEDWGKVYDEAWERIDKKVEGLSKHIKLEEERRSEDA | |
| | QSKAALTDWLRAKASFVIEGLKEADKDEFCRCELKLQKWYGDLRGKPFAIEAENSILDISGESK | |
| | QYNCAFIWQKDGVKKLNLYLIINYFKGGKLRFKKIKPEAFEANRFYTVINKKSGEIVPMEVNEN | |
| | FDDPNLIILPLAFGKRQGREFIWNDLLSLETGSLKLANGRVIEKTLYNRRTRQDEPALFVALTF | |
| | ERREVLDSSNIKPMNLIGVDRGENIPAVIALTDPEGCPLSRFKDSLGNPTHILRIGESYKEKQR | |
| | TIQAKKEVEQRRAGGYSRKYASKAKNLADDMVRNTARDLLYYAVTQDAMLIFENLSRGFGRQGK | |
| | RTFMAERQYTRMEDWLTAKLAYEGLPSKTYLSKTLAQYTSKTCSNCGFTITSADYDRVLEKLKK | |
| | TATGWMTTINGKELKVEGQITYYNRYKRQNVVKDLSVELDRLSEESVNNDISSWTKGRSGEALS | |
| | LLKKRFSHRPVQEKFVCLNCGFETHADEQAALNIARSWLFLRSQEYKKYQTNKTTGNTDKRAFV | |
| | ETWQSFYRKKLKEVWKPAV | |

A multiplexed pooled PASS assay was performed and analyzed as described in Example 6. As noted in Example 6, CasX proteins were expressed using a relatively weakly-expressing promoter to reduce CasX protein expression and thereby improve the sensitivity of the assay. Samples were tested in duplicate, except for engineered CasX protein 1006, which was tested in quadruplicate. In Tables 25, 26, and 27, below, the results for the CasX 1006 samples are reported in two separate rows, each the average of two samples. Streptococcus pyogenes Cas9 without a guide RNA served as a negative control. CasX 515, CasX 676, and engineered CasX protein 812 were also included as controls.

### Results:

Table 25 provides the level of on-target editing produced by various CasX proteins with mutations relative to CasX 515, ranked from highest to lowest activity.

TABLE 25
Average on-targeting editing activity of engineered
CasX proteins, ranked from highest to lowest

| | Mutation(s) | | Average | SEM |
|---|---|---|---|---|
| Protein | relative | Amino | on-target | on-target |
| Designation | to CasX 515* | acid | TTC PAM | TTC PAM |
| (CasX | (Position. | sequence | editing | editing |
| protein no., | Reference. | SEQ | activity | activity |
| or Cas9) | Alternative) | ID NO | (fraction) | (fraction) |
| 1018 | 9.K.G & 891.S.Q | 28285 | 3.01E−01 | 1.20E−01 |
| 1007 | 304.M.T & 826.V.M | 28650 | 2.76E−01 | 7.86E−02 |
| 1006 | 826.V.M & 891.S.Q | 29011 | 2.38E−01 | 4.11E−02 |
| 987 | 169.L.K & 304.M.T | 28443 | 2.32E−01 | 3.38E−02 |
| 1014 | 4.I.G & 891.S.Q | 28036 | 2.29E−01 | 3.45E−02 |
| 1143 | 4.I.G & 826.V.M | 28035 | 2.29E−01 | 3.44E−02 |
| 1019 | 27.—.R & 171.A.S | 27872 | 2.22E−01 | 2.52E−02 |
| 1029 | 5.K.G & 891.S.Q | 28123 | 2.16E−01 | 2.96E−02 |
| 1006 | 826.V.M & 891.S.Q | 29011 | 2.16E−01 | 3.44E−02 |
| 1015 | 5.—.G & 304.M.T | 28058 | 2.11E−01 | 3.20E−02 |
| 970 | 27.—.R & 891.S.Q | 27898 | 2.09E−01 | 2.61E−02 |
| 1028 | 5.K.G & 304.M.T | 28101 | 2.07E−01 | 3.10E−02 |
| 996 | 171.A.Y & 891.S.Q | 28499 | 2.05E−01 | 3.42E−02 |
| 969 | 27.—.R, 171.A.D, & | 49871 | 2.04E−01 | 2.64E−02 |
| | 224.G.T | | | |
| 984 | 64.R.Q & 891.S.Q | 28396 | 2.03E−01 | 9.13E−02 |
| 1041 | 698.S.R & 891.S.Q | 29022 | 2.01E−01 | 3.17E−02 |
| 1016 | 9.K.G & 171.A.D | 27958 | 1.98E−01 | 3.20E−02 |
| 1000 | 224.G.T & 891.S.Q | 35512 | 1.92E−01 | 2.51E−02 |
| 999 | 224.G.T & 304.M.T | 35402 | 1.92E−01 | 2.90E−02 |
| 986 | 169.L.K & 171.A.S | 28439 | 1.90E−01 | 2.76E−02 |
| 977 | 64.R.Q & 169.L.K | 28368 | 1.90E−01 | 3.12E−02 |
| 792 | 27.—.R & 169.L.K | 27870 | 1.89E−01 | 2.89E−02 |
| 993 | 171.A.D & 224.G.T | 30888 | 1.88E−01 | 2.70E−02 |
| 997 | 171.A.S & 304.M.T | 28511 | 1.87E−01 | 2.83E−02 |
| 1025 | 169.L.K & 891.S.Q | 28465 | 1.87E−01 | 2.99E−02 |
| 1001 | 27.—.R, 169.L.K, & | 49873 | 1.85E−01 | 2.57E−02 |
| | 329.G.K | | | |
| 1040 | 481.E.D & 891.S.Q | 28799 | 1.85E−01 | 3.00E−02 |
| 1004 | 304.M.T & 891.S.Q | 28651 | 1.85E−01 | 3.11E−02 |
| 676 | 27.—.R, 170.L.K, & | | 1.84E−01 | 2.28E−02 |
| | 224.G.S | | | |
| 1031 | 6.—.G & 169.L.K | 28137 | 1.84E−01 | 3.02E−02 |
| 980 | 64.R.Q & 171.A.S | 28370 | 1.80E−01 | 3.06E−02 |
| 981 | 64.R.Q & 304.M.T | 28374 | 1.79E−01 | 2.44E−02 |
| 985 | 169.L.K & 171.A.Y | 28438 | 1.73E−01 | 3.57E−02 |
| 989 | 169.L.Q & 224.G.T | 43373 | 1.73E−01 | 3.20E−02 |
| 992 | 169.L.Q & 887.T.D | 29749 | 1.70E−01 | 3.40E−02 |
| 994 | 171.A.Y & 224.G.T | 34870 | 1.68E−01 | 3.21E−02 |
| 1005 | 826.V.M & 887.T.D | 28925 | 1.68E−01 | 3.17E−02 |
| 983 | 64.R.Q & 887.T.D | 28387 | 1.68E−01 | 1.70E−02 |
| 1026 | 169.L.Q & 826.V.M | 29917 | 1.65E−01 | 3.09E−02 |
| 1009 | 4.I.G & 171.A.D | 27952 | 1.65E−01 | 6.43E−02 |
| 982 | 64.R.Q & 398.Y.T | 28378 | 1.64E−01 | 2.73E−02 |
| 978 | 64.R.Q & 169.L.Q | 29308 | 1.63E−01 | 2.96E−02 |
| 515 | — | 228 | 1.63E−01 | 2.38E−02 |
| 1003 | 9.K.G & 27.—.R | 27865 | 1.61E−01 | 2.28E−02 |
| 1017 | 9.K.G & 224.G.T | 33212 | 1.56E−01 | 2.73E−02 |
| 1020 | 35.R.P & 171.A.D | 27959 | 1.55E−01 | 3.01E−02 |
| 998 | 171.A.S & 826.V.M | 28532 | 1.54E−01 | 2.92E−02 |
| 1010 | 4.I.G & 171.A.Y | 28009 | 1.54E−01 | 2.71E−02 |
| 1022 | 35.R.P & 891.S.Q | 28323 | 1.48E−01 | 2.79E−02 |
| 1038 | 224.G.T & 826.V.M | 35507 | 1.48E−01 | 3.11E−02 |
| 1027 | 5.K.G & 171.A.D | 27954 | 1.42E−01 | 2.32E−02 |
| 1012 | 4.I.G & 398.Y.T | 28018 | 1.40E−01 | 2.10E−02 |
| 971 | 35.R.P & 169.L.Q | 29266 | 1.39E−01 | 2.75E−02 |
| 1032 | 6.—.G & 171.A.D | 27955 | 1.39E−01 | 2.40E−02 |
| 1024 | 169.L.K & 398.Y.T | 28447 | 1.38E−01 | 2.87E−02 |
| 1023 | 64.R.Q & 224.G.T | 34088 | 1.37E−01 | 2.34E−02 |
| 1036 | 171.A.S & 887.T.D | 28524 | 1.37E−01 | 2.38E−02 |
| 988 | 169.L.Q & 171.A.Y | 29371 | 1.36E−01 | 3.36E−02 |
| 1034 | 171.A.Y & 826.V.M | 28498 | 1.36E−01 | 3.05E−02 |
| 1030 | 171.A.D & 398.Y.T | 27973 | 1.35E−01 | 2.55E−02 |
| 1039 | 304.M.W & 398.Y.T | 28661 | 1.29E−01 | 3.28E−02 |
| 1033 | 171.A.Y & 304.M.T | 28477 | 1.25E−01 | 2.29E−02 |
| 1021 | 35.R.P & 304.M.T | 28301 | 1.25E−01 | 1.77E−02 |
| 1011 | 4.I.G & 224.G.T | 31244 | 1.24E−01 | 2.13E−02 |
| 979 | 64.R.Q & 171.A.Y | 28369 | 1.24E−01 | 2.15E−02 |
| 1002 | 5.—.G & 35.R.P | 28048 | 1.20E−01 | 2.31E−02 |
| 1035 | 171.A.S & 398.Y.T | 28515 | 1.14E−01 | 2.07E−02 |
| 851 | 35.R.P & 171.A.Y | 28296 | 1.13E−01 | 2.31E−02 |
| 995 | 171.A.Y & 398.Y.T | 28481 | 1.13E−01 | 1.83E−02 |
| 973 | 35.R.P, 171.A.Y, & | 49872 | 1.12E−01 | 1.96E−02 |
| | 304.M.T | | | |
| 976 | 35.R.P & 887.T.D | 28314 | 9.62E−02 | 2.52E−02 |
| 974 | 35.R.P & 224.G.T | 33512 | 9.00E−02 | 1.52E−02 |
| 1037 | 224.G.T & 398.Y.T | 35422 | 8.30E−02 | 1.35E−02 |
| 812 | 329.G.K | 266 | 7.68E−02 | 1.69E−02 |
| 975 | 35.R.P & 398.Y.T | 28305 | 7.49E−02 | 1.51E−02 |
| 991 | 169.L.Q & 398.Y.T | 29560 | 4.87E−02 | 1.74E−02 |
| Cas9 | n/a | — | 0.00E+00 | 0.00E+00 |

*Positions of mutations are shown relative to a CasX 515 sequence with an N-terminal methionine residue (SEQ ID NO: 49699). Each mutation is indicated by its position, reference sequence, and alt sequence, separated by ‘.’ Insertions are indicated with a ‘—’ in the reference sequence (first position), and deletions with a ‘—’ in the alt sequence (second position). Multiple individual mutations are separated by “&”.

As shown in Table 25, 41 of the tested engineered CasX proteins produced higher levels of on-target editing than did CasX 515; the 41 CasX proteins are bolded in Table 25. Engineered CasX protein 1018 had 9.K.G and 891.S.Q amino acid substitutions and produced the highest level of on-target editing in the assay. The CasX 676 control was more active than CasX 515, and CasX 812 was less active than CasX 515, which is consistent with previous results.

A large number of the tested CasX proteins produced lower levels of on-target editing than CasX 515. This suggests that not all combinations of mutations, including combinations of mutations that were relatively active for on-target editing when introduced into CasX 515 as single mutations (see Example 6), are compatible for producing highly active CasX proteins.

To understand the amino acid residues that may be causal for improving CasX activity, the identity of the mutations in the engineered CasX proteins with two or three mutations resulting in improved on-target editing activity relative to CasX 515 was examined (Table 26).

TABLE 26
Summary of mutations in engineered CasX proteins with
greater on-target editing activity than CasX 515

| | Number of | |
|---|---|---|
| Position of mutation | occurrences | Identity of mutation(s) |
| relative to CasX 515 | of mutations | (Number of engineered CasX |
| (SEQ ID NO: 49699) | at position* | proteins with mutation) |
| 891 | 13 | 891.S.Q |
| 169 | 12 | 169.L.K (8); 169.L.Q (4) |
| 171 | 11 | 171.A.S (4); 171.A.D (4); |
| | | 171.A.Y (3) |
| 304 | 8 | 304.M.T |
| 64 | 7 | 64.R.Q |
| 224 | 6 | 224.G.T |
| 27 | 5 | 27.—.R |
| 826 | 4 | 826.V.M |
| 4 | 3 | 4.I.G |
| 5 | 3 | 5.—.G (1); 5.K.G (2) |
| 887 | 3 | 887.T.D |
| 9 | 2 | 9.K.G |
| 6 | 1 | 6.—.G |
| 481 | 1 | 481.E.D |
| 698 | 1 | 698.S.R |

*Excluding CasX 676.

As shown in Table 26, certain positions were mutated in several members of the set of engineered CasX proteins with higher on-target editing activity than CasX 515. For example, the serine to glutamine substitution at position 891 (891.S.Q), in the TSL domain, was found in 13 members of the engineered CasX proteins with improved on-target editing activity relative to CasX 515. The TSL domain is a dynamic domain involved in coordinating the introduction of the target strand to the RuvC active site, and the substitution of serine for the longer glutamine may allow for additional hydrogen bonding interactions with the target strand and more efficient transfer to the nuclease domain.

One of two substitutions at position 169 (169.L.K or 169.L.Q), in the NTSB domain, were found in 12 members of the engineered CasX proteins with higher on-target editing activity than CasX 515. This position is proximal to the second and third nucleotides of the unwound non-target strand in structures of the non-target strand loading state, and the introduction of either a charged residue or one capable of multiple hydrogen-bonding interactions likely allows for the stabilization of the unwound state and thus more efficient unwinding. It should be noted that 169.L.K was more enriched than 169.L.Q among the engineered CasX proteins with improved on-target editing activity, which suggests that while a polar interaction increases enzymatic activity, a charge-charge interaction is more suitable for this position.

One of three substitutions at position 171 (171.A.S, 171.A.D, or 171.A.Y), also in the NTSB domain, were found in 11 members of the engineered CasX proteins with improved on-target editing activity. Residue 171 is solvent-exposed, so a polar residue is likely more favorable at this position. While the residue is not in a position that interacts with the non-target strand in published structures, the dynamic nature of the NTSB domain may allow these residues to make hydrogen-bonding interactions with the target DNA at some point in the unwinding process. A serine is present at this position in the wild-type CasX 2 (SEQ ID NO: 2) sequence and is an alanine in CasX variants containing the chimeric NTSB from CasX1, meaning that the 171.A.S mutation in particular represents a reversion to a wild-type sequence. Notably, 171.A.Y was also found in several of the variants performing worse than CasX 515, which suggests that a tyrosine at position 171 might create too much steric hindrance for proper hydrogen-bonding interactions with the target DNA.

While the 169.L.K and 27.-.R mutations found in CasX 676 were well-represented among the high activity variants, there were a number of orthogonal mutations with distinct mechanisms that may allow for increased activity without the loss of specificity seen in CasX 676. 891. S.Q in particular was found in a number of top-performing activity variants that also have a higher specificity ratio than CasX 515 (see below).

Table 27, below, provides the level of off-target editing produced by various CasX proteins with two or three mutations relative to CasX 515, ranked from lowest to highest activity.

TABLE 27
Average off-targeting editing activity of engineered
CasX proteins, ranked from lowest to highest

| | | | Average | SEM |
|---|---|---|---|---|
| Protein | Mutation relative | Amino | off-target | off-target |
| Designation | to CasX 515* | acid | TTC PAM | TTC PAM |
| (CasX | (Position. | sequence | editing | editing |
| protein no., | Reference. | SEQ | activity | activity |
| or Cas9) | Alternative) | ID NO | (fraction) | (fraction) |
| Cas9 | n/a | — | 0.00E+00 | 0.00E+00 |
| 812 | 329.G.K | 266 | 4.46E−03 | 3.30E−03 |
| 991 | 169.L.Q & 398.Y.T | 29560 | 4.71E−03 | 3.59E−03 |
| 975 | 35.R.P & 398.Y.T | 28305 | 6.63E−03 | 5.75E−03 |
| 976 | 35.R.P & 887.T.D | 28314 | 8.76E−03 | 6.93E−03 |
| 851 | 35.R.P & 171.A.Y | 28296 | 9.95E−03 | 6.91E−03 |
| 974 | 35.R.P & 224.G.T | 33512 | 9.95E−03 | 6.79E−03 |
| 971 | 35.R.P & 169.L.Q | 29266 | 1.15E−02 | 8.07E−03 |
| 1037 | 224.G.T & 398.Y.T | 35422 | 1.22E−02 | 8.00E−03 |
| 1039 | 304.M.W & 398.Y.T | 28661 | 1.24E−02 | 1.01E−02 |
| 988 | 169.L.Q & 171.A.Y | 29371 | 1.30E−02 | 8.38E−03 |
| 973 | 35.R.P, 171.A.Y, & | 49872 | 1.37E−02 | 9.35E−03 |
| | 304.M.T | | | |
| 995 | 171.A.Y & 398.Y.T | 28481 | 1.53E−02 | 9.56E−03 |
| 1002 | 5.—.G & 35.R.P | 28048 | 1.69E−02 | 1.03E−02 |
| 1009 | 4.I.G & 171.A.D | 27952 | 1.76E−02 | 1.08E−02 |
| 1011 | 4.I.G & 224.G.T | 31244 | 1.80E−02 | 1.12E−02 |
| 1035 | 171.A.S & 398.Y.T | 28515 | 1.86E−02 | 9.86E−03 |
| 989 | 169.L.Q & 224.G.T | 43373 | 1.91E−02 | 1.19E−02 |
| 1018 | 9.K.G & 891.S.Q | 28285 | 1.91E−02 | 1.43E−02 |
| 1033 | 171.A.Y & 304.M.T | 28477 | 1.93E−02 | 1.09E−02 |
| 994 | 171.A.Y & 224.G.T | 34870 | 1.95E−02 | 1.21E−02 |
| 1020 | 35.R.P & 171.A.D | 27959 | 1.98E−02 | 1.24E−02 |
| 1022 | 35.R.P & 891.S.Q | 28323 | 2.04E−02 | 1.10E−02 |
| 979 | 64.R.Q & 171.A.Y | 28369 | 2.09E−02 | 1.24E−02 |
| 1032 | 6.—.G & 171.A.D | 27955 | 2.20E−02 | 1.15E−02 |
| 1027 | 5.K.G & 171.A.D | 27954 | 2.28E−02 | 1.29E−02 |
| 1041 | 698.S.R & 891.S.Q | 29022 | 2.41E−02 | 1.32E−02 |
| 1012 | 4.I.G & 398.Y.T | 28018 | 2.50E−02 | 1.30E−02 |
| 1030 | 171.A.D & 398.Y.T | 27973 | 2.53E−02 | 1.31E−02 |
| 1024 | 169.L.K & 398.Y.T | 28447 | 2.54E−02 | 1.39E−02 |
| 982 | 64.R.Q & 398.Y.T | 28378 | 2.58E−02 | 1.58E−02 |
| 983 | 64.R.Q & 887.T.D | 28387 | 2.59E−02 | 1.06E−02 |
| 1040 | 481.E.D & 891.S.Q | 28799 | 2.63E−02 | 1.30E−02 |
| 1017 | 9.K.G & 224.G.T | 33212 | 2.67E−02 | 1.64E−02 |
| 1001 | 27.—.R, 169.L.K, & | 49873 | 2.74E−02 | 1.31E−02 |
| | 329.G.K | | | |
| 992 | 169.L.Q & 887.T.D | 29749 | 2.78E−02 | 1.47E−02 |
| 1036 | 171.A.S & 887.T.D | 28524 | 2.80E−02 | 1.47E−02 |
| 1003 | 9.K.G & 27.—.R | 27865 | 2.80E−02 | 1.40E−02 |
| 1005 | 826.V.M & 887.T.D | 28925 | 2.81E−02 | 1.41E−02 |
| 978 | 64.R.Q & 169.L.Q | 29308 | 2.83E−02 | 1.48E−02 |
| 1021 | 35.R.P & 304.M.T | 28301 | 2.84E−02 | 1.62E−02 |
| 985 | 169.L.K & 171.A.Y | 28438 | 3.00E−02 | 1.62E−02 |
| 977 | 64.R.Q & 169.L.K | 28368 | 3.03E−02 | 1.46E−02 |
| 1023 | 64.R.Q & 224.G.T | 34088 | 3.05E−02 | 1.81E−02 |
| 1034 | 171.A.Y & 826.V.M | 28498 | 3.07E−02 | 1.24E−02 |
| 1010 | 4.I.G & 171.A.Y | 28009 | 3.17E−02 | 1.59E−02 |
| 1026 | 169.L.Q & 826.V.M | 29917 | 3.18E−02 | 1.44E−02 |
| 1016 | 9.K.G & 171.A.D | 27958 | 3.30E−02 | 1.43E−02 |
| 1038 | 224.G.T & 826.V.M | 35507 | 3.30E−02 | 1.58E−02 |
| 1029 | 5.K.G & 891.S.Q | 28123 | 3.31E−02 | 1.77E−02 |
| 1031 | 6.—.G & 169.L.K | 28137 | 3.38E−02 | 1.65E−02 |
| 980 | 64.R.Q & 171.A.S | 28370 | 3.41E−02 | 1.59E−02 |
| 993 | 171.A.D & 224.G.T | 30888 | 3.43E−02 | 1.68E−02 |
| 1028 | 5.K.G & 304.M.T | 28101 | 3.65E−02 | 1.85E−02 |
| 998 | 171.A.S & 826.V.M | 28532 | 3.68E−02 | 1.63E−02 |
| 515 | — | 228 | 3.83E−02 | 1.46E−02 |
| 999 | 224.G.T & 304.M.T | 35402 | 3.91E−02 | 1.93E−02 |
| 1000 | 224.G.T & 891.S.Q | 35512 | 4.14E−02 | 2.07E−02 |
| 996 | 171.A.Y & 891.S.Q | 28499 | 4.25E−02 | 2.02E−02 |
| 981 | 64.R.Q & 304.M.T | 28374 | 4.30E−02 | 2.20E−02 |
| 1014 | 4.I.G & 891.S.Q | 28036 | 4.63E−02 | 2.06E−02 |
| 986 | 169.L.K & 171.A.S | 28439 | 4.63E−02 | 2.46E−02 |
| 1006 | 826.V.M & 891.S.Q | 29011 | 4.66E−02 | 2.16E−02 |
| 1025 | 169.L.K & 891.S.Q | 28465 | 4.73E−02 | 2.04E−02 |
| 997 | 171.A.S & 304.M.T | 28511 | 4.82E−02 | 2.22E−02 |
| 1015 | 5.—.G & 304.M.T | 28058 | 5.10E−02 | 2.19E−02 |
| 987 | 169.L.K & 304.M.T | 28443 | 5.72E−02 | 2.55E−02 |
| 1006 | 826.V.M & 891.S.Q | 29011 | 5.87E−02 | 2.71E−02 |
| 969 | 27.—.R, 171.A.D, & | 49871 | 5.88E−02 | 2.17E−02 |
| | 224.G.T | | | |
| 1004 | 304.M.T & 891.S.Q | 28651 | 6.11E−02 | 2.26E−02 |
| 1143 | 4.I.G & 826.V.M | 28035 | 6.15E−02 | 2.79E−02 |
| 676 | 27.—.R, 170.L.K, & | 30074 | 6.19E−02 | 2.14E−02 |
| | 224.G.S | | | |
| 1019 | 27.—.R & 171.A.S | 27872 | 6.78E−02 | 1.45E−02 |
| 970 | 27.—.R & 891.S.Q | 27898 | 6.87E−02 | 2.22E−02 |
| 1007 | 304.M.T & 826.V.M | 28650 | 6.99E−02 | 8.52E−02 |
| 984 | 64.R.Q & 891.S.Q | 28396 | 7.16E−02 | 1.01E−01 |
| 792 | 27.—.R & 169.L.K | 27870 | 7.26E−02 | 2.39E−02 |

As shown in Table 27, the majority of the tested CasX proteins with pairs of mutations relative to CasX 515 produced lower levels of off-target editing than did CasX 515; these samples are bolded in Table 27.

Table 28, below, provides the specificity ratio (i.e., the average level of on-targeting editing divided by the average level of off-target editing) of the tested CasX proteins with two or three mutations relative to CasX 515, ranked from the highest to lowest ratio. CasX proteins with higher specificity ratios than CasX 515 are bolded in Table 28.

TABLE 28
Specificity ratios of engineered CasX proteins,
ranked from highest to lowest*

| | | | Specificity | |
|---|---|---|---|---|
| | | | ratio | |
| | | | (average | SEM |
| Protein | Mutation relative | Amino | on-target | off-target |
| Designation | to CasX 515* | acid | activity/ | TTC PAM |
| (CasX | (Position. | sequence | average | editing |
| protein no., | Reference. | (SEQ | off-target | activity |
| or Cas9) | Alternative) | ID NO) | activity) | (fraction) |
| 812 | 329.G.K | 266 | 17.22 | 0.52 |
| 1018 | 9.K.G & 891.S.Q | 28285 | 15.76 | 0.35 |
| 971 | 35.R.P & 169.L.Q | 29266 | 12.09 | 0.5 |
| 851 | 35.R.P & 171.A.Y | 28296 | 11.36 | 0.49 |
| 975 | 35.R.P & 398.Y.T | 28305 | 11.3 | 0.67 |
| 976 | 35.R.P & 887.T.D | 28314 | 10.98 | 0.53 |
| 988 | 169.L.Q & 171.A.Y | 29371 | 10.46 | 0.4 |
| 1039 | 304.M.W & 398.Y.T | 28661 | 10.4 | 0.56 |
| 991 | 169.L.Q & 398.Y.T | 29560 | 10.34 | 0.41 |
| 1009 | 4.I.G & 171.A.D | 27952 | 9.38 | 0.23 |
| 989 | 169.L.Q & 224.G.T | 43373 | 9.06 | 0.44 |
| 974 | 35.R.P & 224.G.T | 33512 | 9.05 | 0.51 |
| 994 | 171.A.Y & 224.G.T | 34870 | 8.62 | 0.43 |
| 1041 | 698.S.R & 891.S.Q | 29022 | 8.34 | 0.39 |
| 973 | 35.R.P, 171.A.Y, & | 49872 | 8.18 | 0.51 |
| | 304.M.T | | | |
| 1020 | 35.R.P & 171.A.D | 27959 | 7.83 | 0.43 |
| 995 | 171.A.Y & 398.Y.T | 28481 | 7.39 | 0.46 |
| 1022 | 35.R.P & 891.S.Q | 28323 | 7.25 | 0.35 |
| 1002 | 5.—.G & 35.R.P | 28048 | 7.1 | 0.41 |
| 1040 | 481.E.D & 891.S.Q | 28799 | 7.03 | 0.33 |
| 1011 | 4.I.G & 224.G.T | 31244 | 6.89 | 0.45 |
| 1037 | 224.G.T & 398.Y.T | 35422 | 6.8 | 0.49 |
| 1001 | 27.—.R, 169.L.K, & | 49873 | 6.75 | 0.34 |
| | 329.G.K | | | |
| 1029 | 5.K.G & 891.S.Q | 28123 | 6.53 | 0.4 |
| 983 | 64.R.Q & 887.T.D | 28387 | 6.49 | 0.38 |
| 1033 | 171.A.Y & 304.M.T | 28477 | 6.48 | 0.31 |
| 982 | 64.R.Q & 398.Y.T | 28378 | 6.36 | 0.45 |
| 1032 | 6.—.G & 171.A.D | 27955 | 6.32 | 0.35 |
| 977 | 64.R.Q & 169.L.K | 28368 | 6.27 | 0.32 |
| 1027 | 5.K.G & 171.A.D | 27954 | 6.23 | 0.4 |
| 1035 | 171.A.S & 398.Y.T | 28515 | 6.13 | 0.35 |
| 992 | 169.L.Q & 887.T.D | 29749 | 6.12 | 0.33 |
| 1016 | 9.K.G & 171.A.D | 27958 | 6 | 0.27 |
| 1005 | 826.V.M & 887.T.D | 28925 | 5.98 | 0.31 |
| 979 | 64.R.Q & 171.A.Y | 28369 | 5.93 | 0.42 |
| 1017 | 9.K.G & 224.G.T | 33212 | 5.84 | 0.44 |
| 985 | 169.L.K & 171.A.Y | 28438 | 5.77 | 0.34 |
| 978 | 64.R.Q & 169.L.Q | 29308 | 5.76 | 0.33 |
| 1003 | 9.K.G & 27.—.R | 27865 | 5.75 | 0.36 |
| 1028 | 5.K.G & 304.M.T | 28101 | 5.67 | 0.36 |
| 1012 | 4.I.G & 398.Y.T | 28018 | 5.6 | 0.37 |
| 993 | 171.A.D & 224.G.T | 30888 | 5.48 | 0.34 |
| 1031 | 6.—.G & 169.L.K | 28137 | 5.44 | 0.34 |
| 1024 | 169.L.K & 398.Y.T | 28447 | 5.43 | 0.32 |
| 1030 | 171.A.D & 398.Y.T | 27973 | 5.34 | 0.33 |
| 980 | 64.R.Q & 171.A.S | 28370 | 5.28 | 0.3 |
| 1026 | 169.L.Q & 826.V.M | 29917 | 5.19 | 0.27 |
| 1006 | 826.V.M & 891.S.Q | 29011 | 5.11 | 0.29 |
| 1014 | 4.I.G & 891.S.Q | 28036 | 4.95 | 0.29 |
| 999 | 224.G.T & 304.M.T | 35402 | 4.91 | 0.34 |
| 1036 | 171.A.S & 887.T.D | 28524 | 4.89 | 0.35 |
| 1010 | 4.I.G & 171.A.Y | 28009 | 4.86 | 0.33 |
| 996 | 171.A.Y & 891.S.Q | 28499 | 4.82 | 0.31 |
| 1000 | 224.G.T & 891.S.Q | 35512 | 4.64 | 0.37 |
| 1023 | 64.R.Q & 224.G.T | 34088 | 4.49 | 0.42 |
| 1038 | 224.G.T & 826.V.M | 35507 | 4.48 | 0.27 |
| 1034 | 171.A.Y & 826.V.M | 28498 | 4.43 | 0.43 |
| 1021 | 35.R.P & 304.M.T | 28301 | 4.4 | 0.18 |
| 515 | — | 228 | 4.26 | 0.24 |
| 998 | 171.A.S & 826.V.M | 28532 | 4.18 | 0.25 |
| 981 | 64.R.Q & 304.M.T | 28374 | 4.16 | 0.37 |
| 1015 | 5.—.G & 304.M.T | 28058 | 4.14 | 0.28 |
| 986 | 169.L.K & 171.A.S | 28439 | 4.1 | 0.39 |
| 987 | 169.L.K & 304.M.T | 28443 | 4.06 | 0.3 |
| 1025 | 169.L.K & 891.S.Q | 28465 | 3.95 | 0.27 |
| 1007 | 304.M.T & 826.V.M | 28650 | 3.95 | 0.93 |
| 997 | 171.A.S & 304.M.T | 28511 | 3.88 | 0.31 |
| 1143 | 4.I.G & 826.V.M | 28035 | 3.72 | 0.3 |
| 1006 | 826.V.M & 891.S.Q | 29011 | 3.68 | 0.3 |
| 969 | 27.—.R, 171.A.D, & | 49871 | 3.47 | 0.24 |
| | 224.G.T | | | |
| 1019 | 27.—.R & 171.A.S | 27872 | 3.27 | 0.1 |
| 970 | 27.—.R & 891.S.Q | 27898 | 3.04 | 0.2 |
| 1004 | 304.M.T & 891.S.Q | 28651 | 3.03 | 0.2 |
| 676 | 27.—.R, 170.L.K, & | 30074 | 2.97 | 0.22 |
| | 224.G.S | | | |
| 984 | 64.R.Q & 891.S.Q | 28396 | 2.84 | 0.95 |
| 792 | 27.—.R & 169.L.K | 27870 | 2.6 | 0.18 |

*Specificity ratio and SEM values are shown rounded to the nearest hundredth.

As shown in Table 28, the majority of the tested engineered CasX proteins had higher on-target to off-target editing ratios than CasX 515. While the previously validated high-specificity variant CasX 812 had the highest specificity ratio, consistent with results described in Example 2, above, many engineered CasX proteins demonstrated high specificity ratios without as significant a loss in on-target activity as was observed for CasX 812.

The 35.R.P mutation was commonly observed in variants with very high specificity ratios. This residue is in the OBD and believed to be involved in binding the guide RNA. Mutation to a proline at this position may have complex effects on allosteric regulation. Notably, these variants also tended to have low activity, suggesting that apparent specificity may be in part the result of less efficient RNP formation due to the disruption of this guide-binding interaction. Overall, an inverse correlation was observed between specificity ratio and activity. This suggests that it is difficult to fully avoid trade-offs between activity and specificity. However, it is also evident that the strategy of combining activity and specificity mutants can compensate for this trade-off and result in variants with both characteristics improved.

Notably, some engineered CasX variants produced both higher levels of on-target editing and lower levels of off-target editing than did CasX 515, namely engineered CasX proteins 977, 978, 980, 982, 983, 985, 989, 992, 993, 994, 1001, 1005, 1009, 1016, 1018, 1026, 1028, 1029, 1031, 1040, and 1041. An even greater number had higher on-target activity and a higher specificity ratio, specifically, engineered CasX proteins 977, 978, 980, 982, 983, 985, 989, 992, 993, 994, 996, 999, 1000, 1001, 1005, 1006, 1009, 1014, 1016, 1018, 1026, 1028, 1029, 1031, 1040, and 1041. Such engineered CasX proteins are therefore interpreted to be highly active and highly specific.

Taken together, the results described herein demonstrate that mutations to CasX 515 can be introduced into the sequence that result in engineered CasX with improved gene editing activity and/or specificity.

### Example 8: Design and Assessment of Modified gRNAs in Improving Editing when Delivered Together with CasX mRNA In Vitro and In Vivo

Experiments were performed to identify new gRNA scaffold sequences and demonstrate that chemical modifications of these gRNA scaffolds enhance the editing efficiency of the CasX:gRNA system when delivered in vitro in conjunction with CasX mRNA as editing pairs.

### Materials and Methods

Synthesis of gRNAs:

All gRNAs tested in this example were chemically synthesized and were derived from gRNA scaffolds 174 and 235 and engineered ribonucleic acid scaffold (ERS) 316. The sequences of gRNA scaffolds 174 and 235 and ERS 316 and their chemical modification profiles are listed in Table 29. The sequences of the resulting gRNAs, including spacers targeting PCSK9, B2M, or ROSA26, and their chemical modification profiles assayed in this example are listed in Table 30. A schematic of the structure of gRNA scaffolds 174 and 235 and ERS 316 are shown in FIGS. 11A-11C, respectively, and the sites of chemical modifications of the gRNAs are shown schematically in FIGS. 8A, 8B, 10, 16A, and 16B.

TABLE 29
Sequences of gRNA scaffolds and ERSs with their different
chemical modification profiles (denoted by version number),
where “NNNNNNNNNNNNNNNNNNNN” is a spacer placeholder.
Chemical modifications: * = phosphorothioate bond; m =
2′OMe modification

| gRNA | | |
|---|---|---|
| scaffold/ | | |
| ERS no. | | SEQ ID |
| (version) | gRNA sequence | NO |
| 174 (v0) | ACUGGCGCUUUUAUCUGAUUACUUUGAGAGCCAUCACCAGCGACUAUGUCGUAGUGGGU | 49749 |
| | AAAGCUCCCUCUUCGGAGGGAGCAUCAAAGNNNNNNNNNNNNNNNNNNNN | |
| 174 (v1) | mA*mC*mU*GGCGCUUUUAUCUGAUUACUUUGAGAGCCAUCACCAGCGACUAUGUCGUA | 49750 |
| | GUGGGUAAAGCUCCCUCUUCGGAGGGAGCAUCAAAGNNNNNNNNNNNNNNNNNmN*mN* | |
| | mN | |
| 174 (v2) | mA*mC*mU*GGCGCUUUUAUCUGAUUACUUUGAGAGCCAUCACCAGCGACUAUGUCGUA | 49751 |
| | GUGGGUAAAGCUCCCUCUUCGGAGGGAGCAUCAAAGNNNNNNNNNNNNNNNNNNNN*mU | |
| | *mU*mU | |
| 174 (v3) | mA*mC*mU*mGmGmCmGmCmUmUmUmUmAmUmCmUmGmAmUUACUUUGmAmGmAmGmCm | 49752 |
| | CmAmUmCmAmCmCAGCGAmCmUAUmGmUmCmGUAGUGmGmGmUmAmAmAmGmCmUmCmC | |
| | mCmUmCmUmUmCmGmGmAmGmGmGmAmGmCmAmUmCmAAAGNNNNNNNNNNNNNNNNNm | |
| | N*mN*mN | |
| 174 (v4) | mA*mC*mU*mGmGmCmGmCUUUUmAmUmCmUmGmAmUUACUUUGmAmGmAmGmCmCmAm | 49753 |
| | UmCmAmCmCAGCGAmCmUAUmGmUmCmGUAGUGmGmGmUmAmAmAmGmCmUmCmCmCmU | |
| | mCmUmUmCmGmGmAmGmGmGmAmGmCmAmUCAAAGNNNNNNNNNNNNNNNNNmN*mN*m | |
| | N | |
| 174 (v5) | mA*mC*mU*GGCGCUUUUAUCUGAUUACUUUGAGAGCCAUCACCAGCGAmCmUAUmGmU | 49754 |
| | mCmGUAGUGGGUAAAmGmCmUmCmCmCmUmCmUmUmCmGmGmAmGmGmGmAmGmCAUCA | |
| | AAGNNNNNNNNNNNNNNNNNmN*mN*mN | |
| 174 (v6) | mA*mC*mU*GGCGCUUUUAUCUGAUUACUUUGAGAGCCAUCACCAGCGACUAUGUCGUA | 49755 |
| | GUGGGUAAAmGmCmUmCmCmCmUmCmUmUmCmGmGmAmGmGmGmAmGmCAUCAAAGNNN | |
| | NNNNNNNNNNNNNNmN*mN*mN | |
| 174 (v7) | mA*mC*mU*GGmCGmCmUUUUAmUmCUGAUUACUUUGmAmGAGCCmAmUmCmAmCCAGC | 49756 |
| | mGmAmCmUAUmGmUmCmGUAGUGGmGmUAmAmAmGmCmUmCmCmCmUmCmUmUmCmGmG | |
| | mAmGmGmGmAmGmCmAmUCAAAGNNNNNNNNNNNNNNNNN*mN*mN*mN | |
| 174 (v8) | mA*mC*mU*GGCGCUUUUAUCUGAUUACUUUGAGAGCCmAmUmCmAmCCAGCmGmAmCm | 49757 |
| | UAUmGmUmCmGUAGUGGmGmUmAmAAmGmCmUmCmCmCmUmCmUmUmCmGmGmAmGmGm | |
| | GmAmGmCmAmUCAAAGNNNNNNNNNNNNNNNNN*mN*mN*mN | |
| 174 (v9) | mA*mC*mU*GGmCmGCmUUUUAmUmCUGAUUACUUUGmAmGAGCCAUCACCAGCmGmAm | 49758 |
| | CmUAUmGmUmCmGUAGUGGGUAAAmGmCmUmCmCmCmUmCmUmUmCmGmGmAmGmGmGm | |
| | AmGmCAUCAAAGNNNNNNNNNNNNNNNNN*mN*mN*mN | |
| 235 (v0) | ACUGGCGCUUCUAUCUGAUUACUCUGAGCGCCAUCACCAGCGACUAUGUCGUAGUGGGU | 49759 |
| | AAAGCCGCUUACGGACUUCGGUCCGUAAGAGGCAUCAGAGNNNNNNNNNNNNNNNNNNN | |
| | N | |
| 235 (v1) | mA*mC*mU*GGCGCUUCUAUCUGAUUACUCUGAGCGCCAUCACCAGCGACUAUGUCGUA | 49760 |
| | GUGGGUAAAGCCGCUUACGGACUUCGGUCCGUAAGAGGCAUCAGAGNNNNNNNNNNNNN | |
| | NNNNmN*mN*mN | |
| 235 (v2) | mA*mC*mU*GGCGCUUCUAUCUGAUUACUCUGAGCGCCAUCACCAGCGACUAUGUCGUA | 49761 |
| | GUGGGUAAAGCCGCUUACGGACUUCGGUCCGUAAGAGGCAUCAGAGNNNNNNNNNNNNN | |
| | NNNNNNN*mU*mU*mU | |
| 235 (v3) | mA*mC*mU*mGmGmCmGmCmUmUmCmUmAmUmCmUmGmAmUUACUCUGmAmGmCmGmCm | 49762 |
| | CmAmUmCmAmCmCAGCGAmCmUAUmGmUmCmGUAGUGmGmGmUmAmAmAmGmCmCmGmC | |
| | mUmUmAmCmGmGmAmCmUmUmCmGmGmUmCmCmGmUmAmAmGmAmGmGmCmAmUmCmAG | |
| | AGNNNNNNNNNNNNNNNNNmN*mN*mN | |
| 235 (v4) | mA*mC*mU*mGmGmCmGmCUUCUmAmUmCmUmGmAmUUACUCUGmAmGmCmGmCmCmAm | 49763 |
| | UmCmAmCmCAGCGAmCmUAUmGmUmCmGUAGUGmGmGmUmAmAmAmGmCmCmGmCmUmU | |
| | mAmCmGmGmAmCmUmUmCmGmGmUmCmCmGmUmAmAmGmAmGmGmCmAmUCAGAGNNNN | |
| | NNNNNNNNNNNNNmN*mN*mN | |
| 235 (v5) | mA*mC*mU*GGCGCUUCUAUCUGAUUACUCUGAGCGCCAUCACCAGCGAmCmUAUmGmU | 49764 |
| | mCmGUAGUGGGUmAmAmAmGmCmCmGmCmUmUmAmCmGmGmAmCmUmUmCmGmGmUmCm | |
| | CmGmUmAmAmGmAmGmGmCAUCAGAGNNNNNNNNNNNNNNNNNmN*mN*mN | |
| 235 (v6) | mA*mC*mU*GGCGCUUCUAUCUGAUUACUCUGAGCGCCAUCACCAGCGACUAUGUCGUA | 49765 |
| | GUGGGUmAmAmAmGmCmCmGmCmUmUmAmCmGmGmAmCmUmUmCmGmGmUmCmCmGmUm | |
| | AmAmGmAmGmGmCAUCAGAGNNNNNNNNNNNNNNNNNmN*mN*mN | |
| 235 (v7) | mA*mC*mU*GGmCGmCmUUCUAmUmCUGAUUACUCUGmAmGCGCCmAmUmCmAmCCAGC | 49766 |
| | mGmAmCmUAUmGmUmCmGUAGUGGmGmUmAmAAmGmCmCmGmCmUmUmAmCmGmGmAmC | |
| | mUmUmCmGmGmUmCmCmGmUmAmAmGmAmGmGmCmAmUCAGAGNNNNNNNNNNNNNNNN | |
| | N*mN*mN*mN | |
| 235 (v8) | mA*mC*mU*GGCGCUUCUAUCUGAUUACUCUGAGCGCCmAmUmCmAmCCAGCmGmAmCm | 49767 |
| | UAUmGmUmCmGUAGUGGmGmUmAmAAmGmCmCmGmCmUmUmAmCmGmGmAmCmUmUmCm | |
| | GmGmUmCmCmGmUmAmAmGmAmGmGmCmAmUCAGAGNNNNNNNNNNNNNNNNN*mN*mN | |
| | *mN | |
| 235 (v9) | mA*mC*mU*GGmCGmCmUUCUAmUmCUGAUUACUCUGmAmGCGCCAUCACCAGCmGmAm | 49768 |
| | CmUAUmGmUmCmGUAGUGGGUAAAmGmCmCmGmCmUmUmAmCmGmGmAmCmUmUmCmGm | |
| | GmUmCmCmGmUmAmAmGmAmGmGmCAUCAGAGNNNNNNNNNNNNNNNNN*mN*mN*mN | |
| 316 (v0) | ACUGGCGCUUCUAUCUGAUUACUCUGAGCGCCAUCACCAGCGACUAUGUCGUAGUGGGU | 49769 |
| | AAAGCUCCCUCUUCGGAGGGAGCAUCAGAGNNNNNNNNNNNNNNNNNNNN | |
| 316 (v1) | mA*mC*mU*GGCGCUUCUAUCUGAUUACUCUGAGCGCCAUCACCAGCGACUAUGUCGUA | 49770 |
| | GUGGGUAAAGCUCCCUCUUCGGAGGGAGCAUCAGAGNNNNNNNNNNNNNNNNN*mN*mN | |
| | *mN | |
| 316 (v2) | mA*mC*mU*GGCGCUUCUAUCUGAUUACUCUGAGCGCCAUCACCAGCGACUAUGUCGUA | 49771 |
| | GUGGGUAAAGCUCCCUCUUCGGAGGGAGCAUCAGAGNNNNNNNNNNNNNNNNNNNN*mU | |
| | *mU*mU | |
| 316 (v3) | mA*mC*mU*mGmGmCmGmCmUmUmCmUmAmUmCmUmGmAmUUACUCUGmAmGmCmGmCm | 49772 |
| | CmAmUmCmAmCmCAGCGAmCmUAUmGmUmCmGUAGUGmGmGmUmAmAmAmGmCmUmCmC | |
| | mCmUmCmUmUmCmGmGmAmGmGmGmAmGmCmAmUmCmAGAGNNNNNNNNNNNNNNNNN* | |
| | mN*mN*mN | |
| 316 (v4) | mA*mC*mU*mGmGmCmGmCUUCUmAmUmCmUmGmAmUUACUCUGmAmGmCmGmCmCmAm | 49773 |
| | UmCmAmCmCAGCGAmCmUAUmGmUmCmGUAGUGmGmGmUmAmAmAmGmCmUmCmCmCmU | |
| | mCmUmUmCmGmGmAmGmGmGmAmGmCmAmUCAGAGNNNNNNNNNNNNNNNNN*mN*mN* | |
| | mN | |
| 316 (v5) | mA*mC*mU*GGCGCUUCUAUCUGAUUACUCUGAGCGCCAUCACCAGCGAmCmUAUmGmU | 49774 |
| | mCmGUAGUGGGUAAAmGmCmUmCmCmCmUmCmUmUmCmGmGmAmGmGmGmAmGmCAUCA | |
| | GAGNNNNNNNNNNNNNNNNN*mN*mN*mN | |
| 316 (v6) | mA*mC*mU*GGCGCUUCUAUCUGAUUACUCUGAGCGCCAUCACCAGCGACUAUGUCGUA | 49775 |
| | GUGGGUAAAmGmCmUmCmCmCmUmCmUmUmCmGmGmAmGmGmGmAmGmCAUCAGAGNNN | |
| | NNNNNNNNNNNNNN*mN*mN*mN | |
| 316 (v7) | mA*mC*mU*GGmCGmCmUUCUAmUmCUGAUUACUCUGmAmGCGCCmAmUmCmAmCCAGC | 49776 |
| | mGmAmCmUAUmGmUmCmGUAGUGGmGmUmAmAAmGmCmUmCmCmCmUmCmUmUmCmGmG | |
| | mAmGmGmGmAmGmCmAmUCAGAGNNNNNNNNNNNNNNNNN*mN*mN*mN | |
| 316 (v8) | mA*mC*mU*GGCGCUUCUAUCUGAUUACUCUGAGCGCCmAmUmCmAmCCAGCmGmAmCm | 49777 |
| | UAUmGmUmCmGUAGUGGmGmUmAmAAmGmCmUmCmCmCmUmCmUmUmCmGmGmAmGmGm | |
| | GmAmGmCmAmUCAGAGNNNNNNNNNNNNNNNNN*mN*mN*mN | |
| 316 (v9) | mA*mC*mU*GGmCGmCmUUCUAmUmCUGAUUACUCUGmAmGCGCCAUCACCAGCmGmAm | 49749 |
| | CmUAUmGmUmCmGUAGUGGGUAAAmGmCmUmCmCmCmUmCmUmUmCmGmGmAmGmGmGm | |
| | AmGmCAUCAGAGNNNNNNNNNNNNNNNNN*mN*mN*mN | |

TABLE 30
Sequences of gRNAs with their different chemical modification
profiles (denoted by version number) assayed in this example.
Chemical modifications: * = phosphorothioate bond; m = 2′OMe
modification

| gRNA ID | | | |
|---|---|---|---|
| (scaffold/ERS | | | SEQ ID |
| no .- spacer) | Target | gRNA sequence | NO |
| 174-6.7 | human | ACUGGCGCUUUUAUCUGAUUACUUUGAGAGCCAUCACCAGCGACUAU | 501 |
| (v0) | PCSK9 | GUCGUAGUGGGUAAAGCUCCCUCUUCGGAGGGAGCAUCAAAGUCCUG | |
| | | GCUUCCUGGUGAAGA | |
| 174-6.7 | human | mA*mC*mU*GGCGCUUUUAUCUGAUUACUUUGAGAGCCAUCACCAGC | 502 |
| (v1) | PCSK9 | GACUAUGUCGUAGUGGGUAAAGCUCCCUCUUCGGAGGGAGCAUCAAA | |
| | | GUCCUGGCUUCCUGGUGAmA*mG*mA | |
| 174-6.8 | human | ACUGGCGCUUUUAUCUGAUUACUUUGAGAGCCAUCACCAGCGACUAU | 503 |
| (v0) | PCSK9 | GUCGUAGUGGGUAAAGCUCCCUCUUCGGAGGGAGCAUCAAAGUGGCU | |
| | | UCCUGGUGAAGAUGA | |
| 174-6.8 | human | mA*mC*mU*GGCGCUUUUAUCUGAUUACUUUGAGAGCCAUCACCAGC | 504 |
| (v1) | PCSK9 | GACUAUGUCGUAGUGGGUAAAGCUCCCUCUUCGGAGGGAGCAUCAAA | |
| | | GUGGCUUCCUGGUGAAGAmU*mG*mA | |
| 174-7.9 | human | ACUGGCGCUUUUAUCUGAUUACUUUGAGAGCCAUCACCAGCGACUAU | 505 |
| (v0) | B2M | GUCGUAGUGGGUAAAGCUCCCUCUUCGGAGGGAGCAUCAAAGGUGUA | |
| | | GUACAAGAGAUAGAA | |
| 174-7.9 | human | mA*mC*mU*GGCGCUUUUAUCUGAUUACUUUGAGAGCCAUCACCAGC | 506 |
| (v1) | B2M | GACUAUGUCGUAGUGGGUAAAGCUCCCUCUUCGGAGGGAGCAUCAAA | |
| | | GGUGUAGUACAAGAGAUAmG*mA*mA | |
| 316-6.7 | human | ACUGGCGCUUCUAUCUGAUUACUCUGAGCGCCAUCACCAGCGACUAU | 507 |
| (v0) | PCSK9 | GUCGUAGUGGGUAAAGCUCCCUCUUCGGAGGGAGCAUCAGAGUCCUG | |
| | | GCUUCCUGGUGAAGA | |
| 316-6.7 | human | mA*mC*mU*GGCGCUUCUAUCUGAUUACUCUGAGCGCCAUCACCAGC | 508 |
| (vl’) | PCSK9 | GACUAUGUCGUAGUGGGUAAAGCUCCCUCUUCGGAGGGAGCAUCAGA | |
| | | GUCCUGGCUUCCUGGUGAmA*mG*mA | |
| 316-6.8 | human | ACUGGCGCUUCUAUCUGAUUACUCUGAGCGCCAUCACCAGCGACUAU | 509 |
| (v0) | PCSK9 | GUCGUAGUGGGUAAAGCUCCCUCUUCGGAGGGAGCAUCAGAGUGGCU | |
| | | UCCUGGUGAAGAUGA | |
| 316-6.8 | human | mA*mC*mU*GGCGCUUCUAUCUGAUUACUCUGAGCGCCAUCACCAGC | 510 |
| (v1’) | PCSK9 | GACUAUGUCGUAGUGGGUAAAGCUCCCUCUUCGGAGGGAGCAUCAGA | |
| | | GUGGCUUCCUGGUGAAGAmU*mG*mA | |
| 316-7.9 | human | ACUGGCGCUUCUAUCUGAUUACUCUGAGCGCCAUCACCAGCGACUAU | 511 |
| (v0) | B2M | GUCGUAGUGGGUAAAGCUCCCUCUUCGGAGGGAGCAUCAGAGGUGUA | |
| | | GUACAAGAGAUAGAA | |
| 316-7.9 | human | mA*mC*mU*GGCGCUUCUAUCUGAUUACUCUGAGCGCCAUCACCAGC | 512 |
| (v1’) | B2M | GACUAUGUCGUAGUGGGUAAAGCUCCCUCUUCGGAGGGAGCAUCAGA | |
| | | GGUGUAGUACAAGAGAUAmG*mA*mA | |
| 174-7.37 | human | ACUGGCGCUUUUAUCUgAUUACUUUGAGAGCCAUCACCAGCGACUAU | 513 |
| (v0) | B2M | GUCGUAgUGGGUAAAGCUCCCUCUUCGGAGGGAGCAUCAAAGGGCCG | |
| | | AGAUGUCUCGCUC | |
| 174-7.37 | human | mA*mC*mU*GGCGCUUUUAUCUgAUUACUUUGAGAGCCAUCACCAGC | 49778 |
| (v1*) | B2M | GACUAUGUCGUAgUGGGUAAAGCUCCCUCUUCGGAGGGAGCAUCAAA | |
| | | GGGCCGAGAUGUCUCG*mC*mU*mC | |
| 235-6.7 | human | ACUGGCGCUUCUAUCUGAUUACUCUGAGCGCCAUCACCAGCGACUAU | 515 |
| (v0) | PCSK9 | GUCGUAGUGGGUAAAGCCGCUUACGGACUUCGGUCCGUAAGAGGCAU | |
| | | CAGAGUCCUGGCUUCCUGGUGAAGA | |
| 235-6.7 | human | mA*mC*mU*GGCGCUUCUAUCUGAUUACUCUGAGCGCCAUCACCAGC | 516 |
| (v1) | PCSK9 | GACUAUGUCGUAGUGGGUAAAGCCGCUUACGGACUUCGGUCCGUAAG | |
| | | AGGCAUCAGAGUCCUGGCUUCCUGGUGAmA*mG*mA | |
| 235-6.7 | human | mA*mC*mU*GGCGCUUCUAUCUGAUUACUCUGAGCGCCAUCACCAGC | 517 |
| (v2) | PCSK9 | GACUAUGUCGUAGUGGGUAAAGCCGCUUACGGACUUCGGUCCGUAAG | |
| | | AGGCAUCAGAGUCCUGGCUUCCUGGUGAAGAU*mU*mU*mU | |
| 235-6.7 | human | mA*mC*mU*mGmGmCmGmCmUmUmCmUmAmUmCmUmGmAmUUACUCU | 518 |
| (v3) | PCSK9 | GmAmGmCmGmCmCmAmUmCmAmCmCAGCGAmCmUAUmGmUmCmGUAG | |
| | | UGmGmGmUmAmAmAmGmCmCmGmCmUmUmAmCmGmGmAmCmUmUmCm | |
| | | GmGmUmCmCmGmUmAmAmGmAmGmGmCmAmUmCmAGAGUCCUGGCUU | |
| | | CCUGGUGAmA*mG*mA | |
| 235-6.7 | human | mA*mC*mU*mGmGmCmGmCUUCUmAmUmCmUmGmAmUUACUCUGmAm | 519 |
| (v4) | PCSK9 | GmCmGmCmCmAmUmCmAmCmCAGCGAmCmUAUmGmUmCmGUAGUGmG | |
| | | mGmUmAmAmAmGmCmCmGmCmUmUmAmCmGmGmAmCmUmUmCmGmGm | |
| | | UmCmCmGmUmAmAmGmAmGmGmCmAmUCAGAGUCCUGGCUUCCUGGU | |
| | | GAmA*mG*mA | |
| 235-6.7 | human | mA*mC*mU*GGCGCUUCUAUCUGAUUACUCUGAGCGCCAUCACCAGC | 520 |
| (v5) | PCSK9 | GAmCmUAUmGmUmCmGUAGUGGGUmAmAmAmGmCmCmGmCmUmUmAm | |
| | | CmGmGmAmCmUmUmCmGmGmUmCmCmGmUmAmAmGmAmGmGmCAUCA | |
| | | GAGUCCUGGCUUCCUGGUGAmA*mG*mA | |
| 235-6.7 | human | mA*mC*mU*GGCGCUUCUAUCUGAUUACUCUGAGCGCCAUCACCAGC | 521 |
| (v6) | PCSK9 | GACUAUGUCGUAGUGGGUmAmAmAmGmCmCmGmCmUmUmAmCmGmGm | |
| | | AmCmUmUmCmGmGmUmCmCmGmUmAmAmGmAmGmGmCAUCAGAGUCC | |
| | | UGGCUUCCUGGUGAmA*mG*mA | |
| 235-6.8 | human | ACUGGCGCUUCUAUCUGAUUACUCUGAGCGCCAUCACCAGCGACUAU | 522 |
| (v0) | PCSK9 | GUCGUAGUGGGUAAAGCCGCUUACGGACUUCGGUCCGUAAGAGGCAU | |
| | | CAGAGUGGCUUCCUGGUGAAGAUGA | |
| 235-6.8 | human | mA*mC*mU*GGCGCUUCUAUCUGAUUACUCUGAGCGCCAUCACCAGC | 523 |
| (v1) | PCSK9 | GACUAUGUCGUAGUGGGUAAAGCCGCUUACGGACUUCGGUCCGUAAG | |
| | | AGGCAUCAGAGUGGCUUCCUGGUGAAGAmU*mG*mA | |
| 235-6.8 | human | mA*mC*mU*GGCGCUUCUAUCUGAUUACUCUGAGCGCCAUCACCAGC | 524 |
| (v2) | PCSK9 | GACUAUGUCGUAGUGGGUAAAGCCGCUUACGGACUUCGGUCCGUAAG | |
| | | AGGCAUCAGAGUGGCUUCCUGGUGAAGAUGA*mU*mU*mU | |
| 235-6.8 | human | mA*mC*mU*mGmGmCmGmCmUmUmCmUmAmUmCmUmGmAmUUACUCU | 49779 |
| (v3) | PCSK9 | GmAmGmCmGmCmCmAmUmCmAmCmCAGCGAmCmUAUmGmUmCmGUAG | |
| | | UGmGmGmUmAmAmAmGmCmCmGmCmUmUmAmCmGmGmAmCmUmUmCm | |
| | | GmGmUmCmCmGmUmAmAmGmAmGmGmCmAmUmCmAGAGUGGCUUCCU | |
| | | GGUGAAGAmU*mG*mA | |
| 235-6.8 | human | mA*mC*mU*mGmGmCmGmCUUCUmAmUmCmUmGmAmUUACUCUGmAm | 526 |
| (v4) | PCSK9 | GmCmGmCmCmAmUmCmAmCmCAGCGAmCmUAUmGmUmCmGUAGUGmG | |
| | | mGmUmAmAmAmGmCmCmGmCmUmUmAmCmGmGmAmCmUmUmCmGmGm | |
| | | UmCmCmGmUmAmAmGmAmGmGmCmAmUCAGAGUGGCUUCCUGGUGAA | |
| | | GAmU*mG*mA | |
| 235-6.8 | human | mA*mC*mU*GGCGCUUCUAUCUGAUUACUCUGAGCGCCAUCACCAGC | 527 |
| (v5) | PCSK9 | GAmCmUAUmGmUmCmGUAGUGGGUmAmAmAmGmCmCmGmCmUmUmAm | |
| | | CmGmGmAmCmUmUmCmGmGmUmCmCmGmUmAmAmGmAmGmGmCAUCA | |
| | | GAGUGGCUUCCUGGUGAAGAmU*mG*mA | |
| 235-6.8 | human | mA*mC*mU*GGCGCUUCUAUCUGAUUACUCUGAGCGCCAUCACCAGC | 528 |
| (v6) | PCSK9 | GACUAUGUCGUAGUGGGUmAmAmAmGmCmCmGmCmUmUmAmCmGmGm | |
| | | AmCmUmUmCmGmGmUmCmCmGmUmAmAmGmAmGmGmCAUCAGAGUGG | |
| | | CUUCCUGGUGAAGAmU*mG*mA | |
| 316-27.107 | mouse | ACUGGCGCUUCUAUCUGAUUACUCUGAGCGCCAUCACCAGCGACUAU | 529 |
| (v0) | PCSK9 | GUCGUAGUGGGUAAAGCUCCCUCUUCGGAGGGAGCAUCAGAGCUGGC | |
| | | UUCUUGGUGAAGAUG | |
| 316-27.107 | mouse | mA*mC*mU*GGCGCUUCUAUCUGAUUACUCUGAGCGCCAUCACCAGC | 49780 |
| (v1) | PCSK9 | GACUAUGUCGUAGUGGGUAAAGCUCCCUCUUCGGAGGGAGCAUCAGA | |
| | | GCUGGCUUCUUGGUGAAG*mA*mU*mG | |
| 316-27.107 | mouse | mA*mC*mU*GGmCGmCmUUCUAmUmCUGAUUACUCUGmAmGCGCCmA | 530 |
| (v7) | PCSK9 | mUmCmAmCCAGCmGmAmCmUAUmGmUmCmGUAGUGGmGmUmAmAAmG | |
| | | mCmUmCmCmCmUmCmUmUmCmGmGmAmGmGmGmAmGmCmAmUCAGAG | |
| | | CUGGCUUCUUGGUGAAG*mA*mU*mG | |
| 316-27.107 | mouse | mA*mC*mU*GGCGCUUCUAUCUGAUUACUCUGAGCGCCmAmUmCmAm | 531 |
| (v8) | PCSK9 | CCAGCmGmAmCmUAUmGmUmCmGUAGUGGmGmUmAmAAmGmCmUmCm | |
| | | CmCmUmCmUmUmCmGmGmAmGmGmGmAmGmCmAmUCAGAGCUGGCUU | |
| | | CUUGGUGAAG*mA*mU*mG | |
| 316-27.107 | mouse | mA*mc*mU*GGmCGmCmUUCUAmUmCUGAUUACUCUGmAmGCGCCAU | 532 |
| (v9*) | PCSK9 | CACCAGCmGmAmCmUAUmGmUmCmGUAGUGGGUAAAmGmCmUmCmCm | |
| | | CmUmCmUmUmCmGmGmAmGmGmGmAmGmCAUCAGAGCUGGCUUCUUG | |
| | | GUGAA*mG*mA*mU*mG | |
| 174-35.2 (v0) | ROSA26 | ACUGGCGCUUUUAUCUGAUUACUUUGAGAGCCAUCACCAGCGACUAU | 49781 |
| | | GUCGUAGUGGGUAAAGCUCCCUCUUCGGAGGGAGCAUCAAAGAGAAG | |
| | | AUGGGCGGGAGUCUU | |
| 174-35.2 (v2) | ROSA26 | mA*mC*mU*GGCGCUUUUAUCUGAUUACUUUGAGAGCCAUCACCAGC | 49782 |
| | | GACUAUGUCGUAGUGGGUAAAGCUCCCUCUUCGGAGGGAGCAUCAAA | |
| | | GAGAAGAUGGGCGGGAGUCUU*mU*mU*mU | |
| 316-35.2 (v0) | ROSA26 | ACUGGCGCUUCUAUCUGAUUACUCUGAGCGCCAUCACCAGCGACUAU | 49783 |
| | | GUCGUAGUGGGUAAAGCUCCCUCUUCGGAGGGAGCAUCAGAGAGAAG | |
| | | AUGGGCGGGAGUCUU | |
| 316-35.2 (v1) | ROSA26 | mA*mC*mU*GGCGCUUCUAUCUGAUUACUCUGAGCGCCAUCACCAGC | 49784 |
| | | GACUAUGUCGUAGUGGGUAAAGCUCCCUCUUCGGAGGGAGCAUCAGA | |
| | | GAGAAGAUGGGCGGGAGU*mC*mU*mU | |
| 316-35.2 (v5) | ROSA26 | mA*mC*mU*GGCGCUUCUAUCUGAUUACUCUGAGCGCCAUCACCAGC | 49785 |
| | | GAmCmUAUmGmUmCmGUAGUGGGUAAAmGmCmUmCmCmCmUmCmUmU | |
| | | mCmGmGmAmGmGmGmAmGmCAUCAGAGAGAAGAUGGGCGGGAGU*mC | |
| | | *mU*mU | |

Note that gRNAs annotated with a v1’ design contain one less phosphorothioate bond on the 3′ end of the gRNA. gRNAs annotated with v1* contain one extra phosphorothioate bond on the 3′ end of the gRNA. gRNAs annotated with a v9* contain an extra phosphorothioate bond on the 3′ end of the gRNA.

Biochemical Characterization of gRNA Activity:

Target DNA oligonucleotides with fluorescent moieties on the 5′ ends were purchased commercially (sequences listed in Table 31). Double-stranded DNA (dsDNA) targets were formed by mixing the oligos in a 1:1 ratio in 1× cleavage buffer (20 mM Tris HCl pH 7.5, 150 mM NaCl, 1 mM TCEP, 5% glycerol, 10 mM MgCl₂), following by heating to 95° C. for 10 minutes, and then allowing the solution to cool to room temperature. CasX ribonucleoproteins (RNPs) were reconstituted with CasX 491 and the indicated gRNAs at a final concentration of 1 μM with 1.2-fold excess of the indicated gRNA in 1× cleavage buffer. RNPs were allowed to form at 37° C. for 10 minutes.

The effects of various structural and chemical modifications to the gRNA scaffold on the cleavage rate of CasX 491 RNPs were determined. Cleavage reactions were prepared with final RNP concentrations of 200 nM and final target concentrations of 10 nM, and reactions were carried out at 16° C. and initiated by the addition of the labeled target DNA substrate (Table 31). Aliquots of reactions were taken at 0.25, 0.5, 1, 2, 5, and 10 minutes and quenched by adding an equal volume of 95% formamide with 20 mM EDTA. Samples were denatured at 95° C. for 10 minutes and resolved on a 10% urea-PAGE gel. Gels were imaged on a Typhoon™ laser-scanner platform and quantified using ImageQuant™ TL 8.2 image analysis software (Cytiva™). The apparent first-order rate constant of non-target strand cleavage (k_{cleave-}) was determined for each CasX:gRNA combination.

To determine the competent fraction formed by each gRNA, cleavage reactions were prepared with final RNP concentrations of 100 nM and final target concentrations of 100 nM. Reactions were carried out at 37° C. and initiated by the addition of the labeled target substrate (Table 31). Aliquots were taken at 0.5, 1, 2, 5, 10, and 30 minutes and quenched by adding an equal volume of 95% formamide with 25 mM EDTA. Samples were denatured by heating at 95° C. for 10 minutes and resolved on a 10% urea-PAGE gel. Gels were imaged and quantified as above. CasX was assumed to act as a single-turnover enzyme under the assayed conditions, as indicated by the observation that sub-stoichiometric amounts of enzyme would fail to cleave a greater-than-stoichiometric amount of target substrate even under extended time-scales, and instead would approach a plateau that scaled with the amount of enzyme present. Thus, the fraction of target substrate cleaved over long time-scales by an equimolar amount of RNP would be indicative of the fraction of RNP that was properly formed and active for cleavage. The cleavage traces were fitted with a biphasic rate model, as the cleavage reaction clearly deviated from monophasic under this concentration regime. The plateau of each fit was determined and reported as the active fraction for each RNP in Table 34.

TABLE 31
Sequences of target DNA substrate oligonucleotides with
fluorescent moieties on the 5′ ends used for biochemical
characterization of gRNA activity.

| DNA substrate | Sequence |
|---|---|
| 6.7/6.8 target top strand | /700/CATGTCTTCCATGGCCTTCTTCCTGGCT |
| (SEQ ID NO: 533) | TCCTGGTGAAGATGAGTGGCGACCTGCTGGAG |
| 6.7/6.8 target bottom strand | /800/CTCCAGCAGGTCGCCACTCATCTTCACC |
| (SEQ ID NO: 534) | AGGAAGCCAGGAAGAAGGCCATGGAAGACATG |

/700/ = IRDye700;
/800/ = IRDye800

In Vitro Transcription of CasX mRNA:

DNA templates encoding CasX 491 or CasX 676 (see Table 32 for encoding sequences) used for in vitro transcription were generated by PCR using forward primers containing a T7 promoter, followed by agarose gel extraction of the appropriately sized DNA. 25 ng/μL final concentration of template DNA was used in each in vitro transcription reaction that was carried out following the manufacturer's recommended protocol with slight modifications. Following in vitro transcription reaction incubation for 2-3 hours at 37° C., which were carried out with CleanCap® AG and N1-methyl-pseudouridine, DNAse digestion of template DNA and column-based purification using the Zymo RNA miniprep kit were performed. The poly(A) tail was added using E. coli PolyA Polymerase following the manufacturer's protocol, followed by column-based purification as stated above. Poly(A) tailed in vitro transcribed RNA was eluted in RNAse free water, analyzed on an Agilent TapeStation for integrity, and flash frozen prior to storage at −80° C.

TABLE 32
Encoding sequences of the CasX mRNA
molecules assessed in this example*

| CasX 491 | | SEQ |
|---|---|---|
| mRNA ID | Component (ID) | ID NO |
| CasX 491 | 5′UTR | 49786 |
| mRNA #1 | START codon + c-MYC NLS + linker | 49787 |
| | CasX 491 | 49788 |
| | Linker + c-MYC NLS | 49789 |
| | P2A mScarlet + STOP codon | 49790 |
| CasX 676 | 5′UTR + Kozak sequence | 49791 |
| mRNA #2 | START codon + c-MYC NLS | 49792 |
| | CasX 676 | 49793 |
| | c-MYC NLS + STOP codons | 49794 |
| | 3′UTR | 49795 |
| | Xbal restriction site (partial) | 49796 |
| | Poly(A) tail | 49797 |

*Components are listed in a 5′ to 3′ order within the constructs

In Vitro Delivery of gRNA and CasX mRNA Via Transfection:

Editing at the PCSK9 locus and consequential effects on secreted PCSK9 levels were assessed for conditions using CasX 491 mRNA co-delivered with a PCSK9-targeting gRNA with scaffold 174 compared to conditions where a PCSK9-targeting gRNA with ERS 316 was used. 100 ng of in vitro transcribed mRNA coding for CasX 491 with a P2A and mScarlet fluorescent protein was transfected into HepG2 cells with version 1 (v1) of gRNAs 174-6.7, 174-6.8, 316-6.7, and 316-6.8 (see Table 30) using lipofectamine. After a media change, the following were harvested at 28 hours post-transfection: 1) transfected cells were harvested for editing assessment at the PCSK9 locus by NGS; 2) media supernatant was harvested to measure secreted PCSK9 protein levels by ELISA. For editing analysis by NGS, amplicons were amplified from 200 ng of extracted gDNA with a set of primers targeting the PCSK9 locus and processed for sequencing. Specifically, genomic DNA (gDNA) from harvested cells was extracted using the Zymo Quick-DNA™ Miniprep Plus kit following the manufacturer's instructions. Target amplicons were formed by amplifying regions of interest from 50-100 ng of extracted gDNA with a set of primers targeting the human PCSK9 locus. These gene-specific primers contained an additional sequence at the 5′ ends to introduce Illumina reads 1 and 2 sequences. Further, they contained a 16-nucleotide random sequence that functioned as a unique molecular identifier (UMI). The quality and quantification of the amplicon was assessed using a Fragment Analyzer DNA analyzer kit (Agilent, dsDNA 35-1500 bp). Amplicons were sequenced on the Illumina MiSeq™ according to the manufacturer's instructions. Raw fastq sequencing files were processed by trimming for quality and adapter sequences and merging read 1 and read 2 into a single insert sequence; insert sequences were then analyzed by the CRISPResso2 (v 2.0.29) program. The percentage of reads modified in a window around the 3′ end of the spacer was determined. The activity of the CasX molecule was quantified as the total percent of reads that contain insertions, substitutions, and/or deletions anywhere within this window for each.

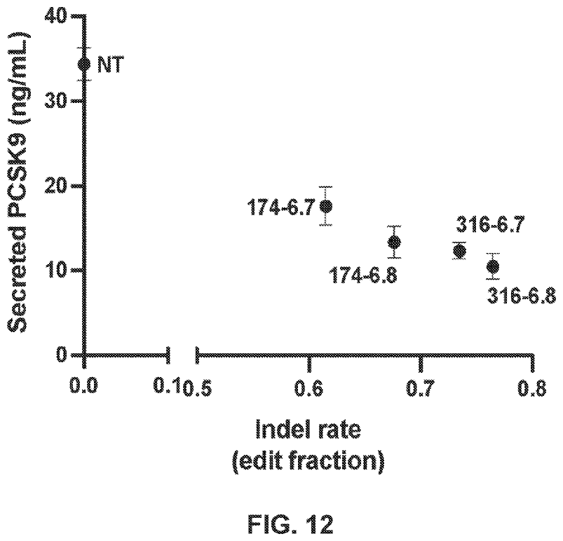

Secreted PCSK9 levels in the media supernatant were also analyzed using a fluorescence resonance energy transfer-based immunoassay from CISBio following the manufacturer's instructions. Here, a gRNA using scaffold 174 with spacer 7.37 (v0; see Table 30), which targeted the endogenous B2M (beta-2-microglobulin) locus, served as the non-targeting (NT) control. These results are shown in FIG. 12.

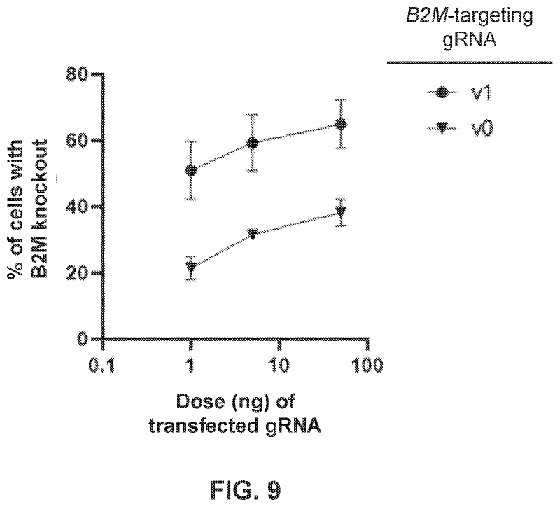

To compare the editing potency of version 0 (v0) and version 1 (vi) of B2M-targeting gRNAs, ˜6E4 HepG2 hepatocytes were seeded per well of a 96-well plate. 24 hours later, seeded cells were co-transfected using lipofectamine with 100 ng of in vitro transcribed mRNA coding for CasX 491 and different doses (1, 5, or 50 ng) of either v0 or vi of the B2M-targeting gRNA containing scaffold 174 and spacer 7.37 (see Table 30). Six days post-transfection, cells were harvested for B2M protein expression analysis via immunostaining of the B2M-dependent HLA protein, followed by flow cytometry using the Attune™ NxT flow cytometer. These results are shown in FIG. 9.

V1 through v6 variants of chemically-modified PCSK9-targeting gRNAs (Table 30) were assessed for their effects on editing potency and consequential effects on secreted PCSK9 levels in vitro. Briefly, 100 ng of in vitro transcribed mRNA coding for CasX protein 491 and a P2A and mScarlet fluorescent protein was transfected into HepG2 cells with 50 ng of the indicated chemically-modified gRNA using lipofectamine. After a media change, the following were harvested at 28 hours post-transfection: 1) transfected cells for editing assessment at the PCSK9 locus by NGS as described above; 2) media supernatant to measure secreted PCSK9 protein levels by ELISA, as described above. Here, a B2M-targeting gRNA was used as a non-targeting control. These results are shown in Table 35.

To formulate LNPs, CasX mRNA and gRNA were encapsulated into LNPs using GenVoy-ILM™ lipids on the Precision NanoSystems Inc. (PNI) Ignite™ Benchtop System and following the manufacturer's guidelines. GenVoy-ILM™ lipids are manufactured by PNI, with a proprietary composition of ionizable lipid:DSPC:cholesterol:stabilizer at 50:10:37.5:2.5 mol %. Briefly, to formulate LNPs, equal mass ratios of CasX mRNA and gRNA were diluted in PNI Formulation Buffer, pH 4.0. GenVoy-ILM™ was diluted 1:1 in anhydrous ethanol. mRNA/gRNA co-formulations were performed using a 6:1 N/P ratio. The RNA and lipids were run through a PNI laminar flow cartridge at a predetermined flow rate ratio (RNA:Genvoy-ILM™) on the PNI Ignite™ Benchtop System. After formulation, the LNPs were diluted in PBS, pH 7.4, to decrease the ethanol concentration and increase the pH, which increases the stability of the particles. Buffer exchange of the mRNA/sgRNA-LNPs was achieved by overnight dialysis into PBS, pH 7.4, at 4° C. using 10k Slide-A-Lyzer™ Dialysis Cassettes (Thermo Scientific™). Following dialysis, the mRNA/gRNA-LNPs were concentrated to >0.5 mg/mL using 100 kDa Amicon®-Ultra Centrifugal Filters (Millipore) and then filter-sterilized. Formulated LNPs were analyzed on a Stunner (Unchained Labs) to determine their diameter and polydispersity index (PDI). Encapsulation efficiency and RNA concentration was determined by RiboGreen™ assay using Invitrogen's Quant-iT™ Ribogreen™ RNA assay kit.

Delivery of LNPs Encapsulating CasX mRNA and Targeting gRNAs In Vitro:

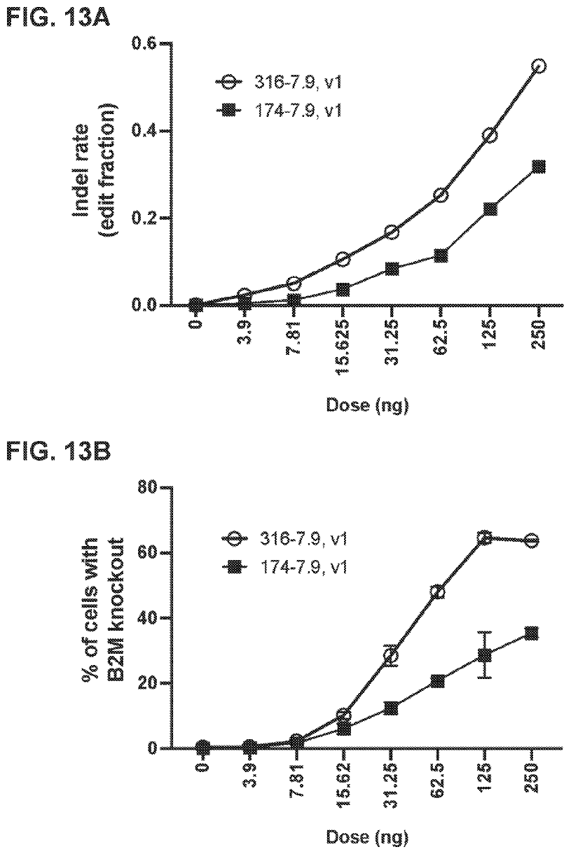

˜50,000 HepG2 cells, cultured in DMEM/F-12 media containing 10% FBS and 1% PenStrep, were seeded per well in a 96-well plate. The next day, seeded cells were treated with varying concentrations of LNPs, which were prepared in six 2-fold serial dilutions starting at 250 ng. These LNPs were formulated to encapsulate CasX 491 mRNA and a B2M-targeting gRNA incorporating either scaffold 174 or ERS 316 with spacer 7.9 (vi; see Table 30). Media was changed 24 hours after LNP treatment, and cells were cultured for six additional days prior to harvesting for gDNA extraction for editing assessment at the B2M locus by NGS and B2M protein expression analysis via HLA immunostaining, followed by flow cytometry using the Attune NxT flow cytometer. Briefly, for editing assessment, amplicons were amplified from 200 ng of extracted gDNA with primers targeting the human B2M locus and processed as described in above. The results of these assays are shown in FIGS. 13A and 13B.

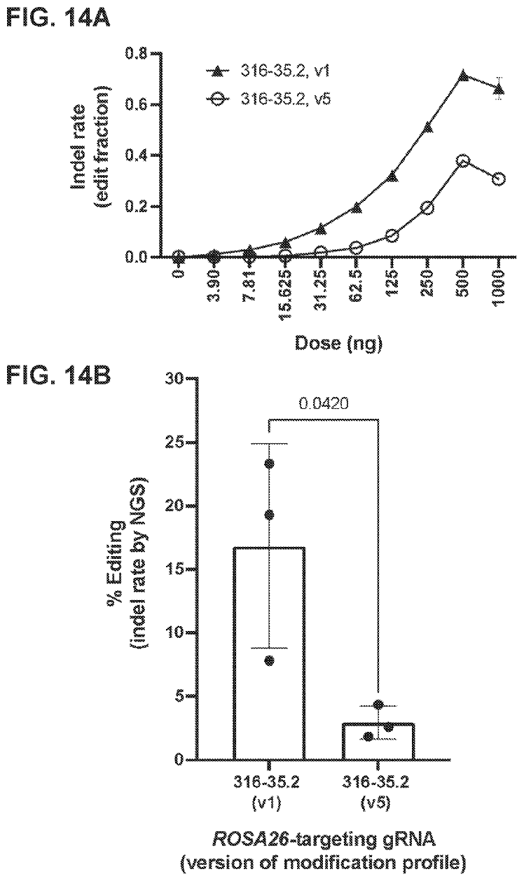

˜20,000 mouse Hepa1-6 hepatocytes were seeded per well in a 96-well plate. The following day, seeded cells were treated with varying concentrations of LNPs, which were prepared in eight 2-fold serial dilutions starting at 1000 ng. These LNPs were formulated to encapsulate CasX 676 mRNA #2 (see Table 32) and a ROSA26-targeting gRNA incorporating ERS 316 with spacer 35.2 (v1 or 5; see Table 30). Media was changed 24 hours post-treatment with LNPs, and cells were cultured for seven additional days prior to harvesting for gDNA extraction for editing assessment at the ROSA26 locus by NGS. Briefly, amplicons were amplified from extracted gDNA with primers targeting the mouse ROSA26 locus and processed as described above. The results of this experiment are shown in FIG. 14A.

Delivery of LNPs Encapsulating CasX mRNA and Targeting gRNA In Vivo:

To assess the effects of using v1 and v5 of ERS 316 in vivo, CasX 676 mRNA #2 (see Table 32) and a ROSA26-targeting gRNA using ERS 316 with spacer 35.2 (v1 or v5; see Table 30) were encapsulated within the same LNP using a 1:1 mass ratio for mRNA:gRNA. LNP co-formulations were generated as described above. Formulated LNPs were buffer-exchanged to PBS for in vivo injection. LNPs were administered intravenously through the retro-orbital sinus into 4-week old C57BL/6 mice. Mice were observed for five minutes after injection to ensure recovery from anesthesia before being placed into their home cage. Naïve, uninjected animals served as experimental controls. Six days post-administration, mice were euthanized, and the liver tissue was harvested for gDNA extraction using the Zymo Research Quick DNA/RNA Miniprep kit following the manufacturer's instructions. Target amplicons were then amplified from the extracted gDNA with a set of primers targeting the mouse ROSA26 locus and processed for editing assessment by NGS as described above. The results of this experiment are shown in FIG. 14B.

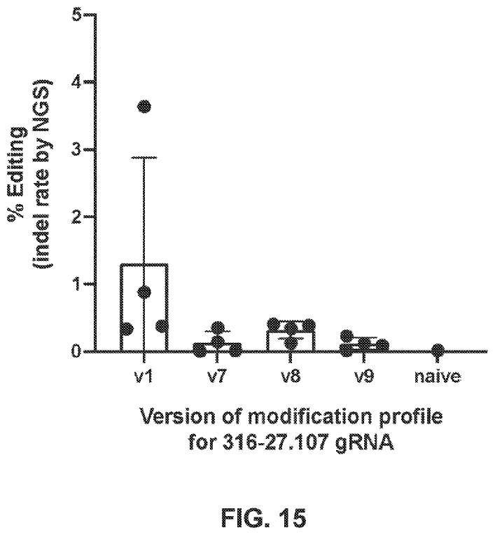

To compare the effects of using v7, v8, and v9 of ERS 316 on editing at the PCSK9 locus in vivo, CasX 676 mRNA #1 (see Table 33 for sequences) and a PCSK9-targeting gRNA using ERS 316 with spacer 27.107 (v1, v7, v8, or v9; see Table 30), were encapsulated within the same LNP using a 1:1 mass ratio for mRNA:gRNA for each gRNA. LNPs were administered retro-orbitally into 6-week old C57BL/6 mice, as described above, and mice were euthanized seven days post-injection to harvest liver tissue for gDNA extraction for editing assessment by NGS at the PCSK9 locus. The results of this experiment are shown in FIG. 15.

TABLE 33
Encoding sequences of CasX 676 mRNA #1 molecule

| CasX ID | Component (ID) | Description | SEQ ID NO |
|---|---|---|---|
| CasX 676 mRNA #1 | 5′UTR | Human HBA | 49798 |
| | START codon + | | 49792 |
| | c-MYC NLS | | |
| | CasX 676 | | 49793 |
| | c-MYC NLS + | | 49794 |
| | STOP codons | | |
| | 3′UTR | Human HBA | 49799 |
| | Poly(A) tail | | 49797 |

*Components are listed in a 5′ to 3′ order within the constructs

### Results:

Assessing the Effects of Various Chemical Modifications on gRNA Activity:

Several studies involving Cas9 have demonstrated that chemical modifications of the gRNA resulted in significantly improved editing activity when delivered with Cas9 mRNA. Following delivery of Cas9 mRNA and gRNA into target cells, unprotected gRNA is susceptible to degradation during the mRNA translation process. Addition of chemical modifications such as 2′O-methyl (2′OMe) groups and phosphorothioate bonds can reduce the susceptibility of the gRNA to cellular RNases, but also have the potential to disrupt folding of the gRNA and its interactions with the CRISPR-Cas protein. Given the lack of structural similarity between CasX and Cas9, as well as their respective gRNAs, appropriate chemical modification profiles must be designed and validated de novo. Using published structures of wild-type CasX from Deltaproteobacteria (PDB codes 6NY1, 6NY2, and 6NY3) as reference, residues that appeared potentially amenable to modification were selected. However, the published structures were of a wild type CasX ortholog and gRNA distinct from the species used as the basis for the engineered variants presented here, and they also lacked the resolution to confidently determine interactions between protein side-chains and the RNA backbone. These limitations introduced a significant amount of ambiguity into determining which nucleotides might be safely modified. As a result, six profiles of chemical modifications (denoted as versions) were designed for initial testing, and these six profiles are illustrated in FIGS. 8A and 8B. The v1 profile was designed as a simple end-protected structure, where the first and last three nucleotides were modified with 2′OMe and phosphorothioate bonds. In the v2 profile, 3′UUU tail was added to mimic the termination sequence used in cellular transcription systems and to move the modified nucleotides outside of the region of the spacer involved in target recognition. The v3 profile included the end protection as in v1, as well as the addition of 2′OMe modifications at all nucleotides identified to be potentially modifiable based on structural analysis. The v4 profile was modeled based on v3, but with all the modifications in the triplex region removed, as this structure was predicted to be more sensitive to any perturbation of the RNA helical structure and backbone flexibility. The v5 profile maintained chemical modifications in the scaffold stem and extended stem regions, while the v6 profile harbored modifications only in the extended stem. The extended stem is a region that would become fully exposed to solvent in the RNP and is amenable to replacement by other hairpin structures, and therefore presumably relatively insensitive to chemical modifications.

The minimally modified v1 gRNA was initially assessed against an unmodified gRNA (v0) to determine the potential benefit of such chemical modifications on editing when the gRNA was co-delivered with CasX mRNA to target cells. Modified (vi) and unmodified (v0) B2M-targeting gRNAs with spacer 7.37 were co-transfected with CasX mRNA into HepG2 cells, and editing at the B2M locus was measured by loss of surface presentation of the B2M-dependent HLA complex, as detected by flow cytometry (FIG. 9). The data demonstrate that use of the vi gRNA resulted in substantially greater loss of B2M expression compared to the levels seen with v0 gRNA across the various doses, thereby confirming that end modifications of the gRNA increased CasX-mediated editing activity upon delivery of the CasX mRNA and gRNA.

The broader set of gRNA chemical modification profiles were assessed using PCSK9-targeting gRNAs using scaffold variant 235 and spacers 6.7 and 6.8 to determine whether the additional chemical modifications would be able to support the formation of active RNPs. In vitro cleavage assays described above were performed to determine k_{cleave} and fraction competence for these engineered gRNAs harboring the various chemical modification profiles. The results from these in vitro cleavage assays are shown in Table 34. The data demonstrate that gRNAs with the v3 profiles exhibited no activity, an indication that the addition of some chemical modifications significantly interfered with RNP formation or activity. Adding v4 chemical modifications resulted in a reasonable cleavage rate in the excess RNP condition, but exhibited very low fraction competence. The difference between v3 and v4 modifications confirmed that modifications in the triplex region prevented the formation of any active RNP, either due to the inability of the gRNA to fold properly or a disruption in the gRNA-protein interactions. The reduced fraction competence resulting from appending v4 modifications suggest that while the gRNA was able to successfully assemble with the CasX protein to form a cleavage-competent RNP, a large majority of the gRNA was misfolded, or that the appended chemical modifications reduced the affinity of the gRNA for the CasX protein and impeded the efficiency of RNP formation. Application of the v5 or v6 profiles resulted in competent fractions that were comparable to, but slightly lower than, those obtained for reactions using the v1 and v2 modifications. While the k_{cleave} values were relatively consistent between v5 and v6 gRNAs, both v5 and v6 gRNAs achieved nearly half of the k_{cleave} values for v1 and v2 gRNAs. The reduced k_{cleave} value for v6 gRNA was particularly surprising, given the lack of expected interaction between the gRNA and CasX protein in the modified extended stem. However, for both v5 and v6 gRNAs, it is possible that the reduced flexibility of the gRNA, resulting from the 2′OMe modifications, inhibited structural changes in the RNP required for efficient cleavage, or that the modified initial base-pairs of the hairpin involved in CasX protein interaction had been negatively impacted by the inclusion of the 2′OMe groups.

TABLE 34
Parameters of cleavage activity assessed for CasX
RNPs with the various PCSK9-targeting gRNAs using
scaffold 235 and harboring the indicated chemical
modification profile, denoted by version number

| gRNA | | |
|---|---|---|
| (scaffold no.-spacer, | k_{cleave} | Fraction |
| version no.) | (min⁻¹) | competence |
| 235-6.7, v1 | 0.901 | 0.398 |
| 235-6.8, v1 | 1.36 | 0.398 |
| 235-6.7, v2 | 0.454 | 0.386 |
| 235-6.8, v2 | 2.03 | 0.361 |
| 235-6.7, v3 | 0 | 0 |
| 235-6.8, v3 | 0 | 0 |
| 235-6.7, v4 | 0.434 | 0.031 |
| 235-6.8, v4 | 0.257 | 0.005 |
| 235-6.7, v5 | 0.506 | 0.313 |
| 235-6.8, v5 | 0.680 | 0.388 |
| 235-6.7, v6 | 0.462 | 0.346 |
| 235-6.8, v6 | 0.715 | 0.325 |

The chemically-modified PCSK9-targeting gRNAs based on scaffold 235 were subsequently assessed for editing in a cell-based assay. CasX mRNA and chemically modified PCSK9-targeting gRNAs were co-transfected into HepG2 cells using lipofectamine. Editing levels were measured by indel rate at the PCSK9 locus by NGS and secreted PCSK9 levels by ELISA, and the data are displayed in Table 35. The data demonstrate that use of v3 and v4 gRNAs resulted in minimal editing activity at the PCSK9 locus, consistent with findings from the biochemical in vitro cleavage assays shown in Table 34. Meanwhile, use of v5 and v6 gRNAs resulted in editing levels, measured by indel rate and PCSK9 secretion, that were slightly lower than the levels attained with use of vi and v2 gRNAs (Table 35). Specifically, the results show that use of v1 and v2 gRNAs, which harbored end modifications, resulted in ˜80-85% editing at the PCSK9 locus, indicating that adding chemical modifications to the gRNA ends was sufficient to achieve efficient editing with CasX. While the data demonstrate that use of v5 and v6 gRNAs resulted in efficient editing in vitro, near-saturating levels of editing were observed with use of the v gRNA in this experiment where a single dose of the gRNA was transfected. As a result, the use of a single dose rendered it challenging to assess clearly the effects of the chemical modifications on editing under guide-limiting conditions. Therefore, profiles v1 and v5 were chosen for further testing, as v1 contains the simplest modification profile, and v5 is the most heavily modified profile whose application demonstrated robust activity in vitro (Tables 34 and 35).

TABLE 35
Editing levels measured by indel rate at PCSK9 locus by NGS and
secreted PCSK9 levels by ELISA in HepG2 cells co-transfected
with CasX 491 mRNA and various chemically-modified PCSK9-targeting
gRNAs using scaffold 235 and either spacer 6.7 or 6.8

| Experimental | Indel rate (edit fraction) | | Secreted PCSK9 (ng/mL) | |
|---|---|---|---|---|
| condition | Mean | Stdev | Mean | Stdev |
| CasX mRNA only | 0.0021 | 0.003 | 52 | 14 |
| 235-6.7, v1 | 0.83 | 0.0058 | 18 | 5.7 |
| 235-6.7, v2 | 0.79 | 0.0071 | 21 | 4 |
| 235-6.7, v3 | 0.024 | 0.02 | 48 | 19 |
| 235-6.7, v4 | 0.12 | 0.006 | 34 | 5.5 |
| 235-6.7, v5 | 0.73 | 0.023 | 21 | 9 |
| 235-6.7, v6 | 0.75 | 0.0069 | 22 | 8.8 |
| 235-6.8, v1 | 0.85 | 0.017 | 16 | 4.4 |
| 235-6.8, v2 | 0.83 | 0.0028 | 20 | 1.5 |
| 235-6.8, v3 | 0.023 | 0.0027 | 39 | 2.7 |
| 235-6.8, v4 | 0.088 | 0.0086 | 42 | 10 |
| 235-6.8, v5 | 0.77 | 0.017 | 19 | 1.6 |
| 235-6.8, v6 | 0.78 | 0.014 | 24 | 6.9 |
| Non-targeting ctrl | 0.0019 | 0.0026 | 42 | 12 |

The v1 and v5 profiles were further tested in another cell-based assay to assess their effects on editing efficiency. LNPs were formulated to co-encapsulate CasX mRNA 42 and v1 and v5 chemically-modified ROSA26-targeting gRNAs using the newly-designed ERS 316 (described further in the following sub-section). The “v5” profile was modified slightly for application to ERS 316. Three 2′ OMe modifications in the non-base-paired region immediately 5′ of the extended stem were removed to restrict modifications to the two stemloop regions. Hepa1-6 hepatocytes were treated with the resulting LNPs at various doses and harvested eight days post-treatment to assess editing at the ROSA26 locus, measured as indel rate detected by NGS (FIG. 14A). The data demonstrate that treatment with LNPs delivering the v5 ROSA26-targeting gRNA resulted in markedly lower editing levels across the range of doses compared to the levels achieved with the v1 counterpart (FIG. 14A). There are several possible explanations for the differences in relative activity observed with use of v5 gRNA in FIG. 14A relative to that observed in Table 35. The first and most likely possible explanation is that the single dose used to achieve editing shown in Table 35 was too high to measure differences in activity accurately between use of v5 gRNA and v1 gRNA. It is also possible that the removal of the modifications outside the stemloop motifs in the ERS 316 version of v5 negatively impacted guide activity. While it is possible that these modifications provide stability benefits that outweigh an activity cost imparted by the stemloop modifications, this seems unlikely given that increasing levels of modification have so far resulted in decreased activity. A final possible explanation is that the modifications in the v5 profile might negatively impact LNP formulation or behavior through differential interactions between the modified nucleotide backbone and the ionizable lipid of the LNP, potentially resulting in less efficient gRNA encapsulation or in less efficient gRNA release following internalization.

LNPs co-encapsulating the CasX mRNA #2 and vi and v5 chemically-modified ROSA26-targeting gRNAs based on ERS 316 were further tested in vivo. FIG. 14B shows the results of the editing assay as percent editing measured as indel rate at the ROSA26 locus. The data demonstrate that use of the v5 gRNA resulted in ˜5-fold lower editing compared to that achieved with use of the v1 gRNA, under more relevant testing conditions of in vivo LNP delivery. These findings support the reduced cleavage rate observed biochemically for the v5 gRNA in Table 34, an indication that the v5 modifications have interfered with some aspect of CasX activity. Given the consistent decrease in activity detected in v5 and v6 profiles (Table 34), the reduced editing may be attributed to modifications in the extended stem region. Although the extended stem of the gRNA has minimal interactions with the CasX protein, it is possible that addition of 2′OMe groups at the first base-pair disrupted either the CasX protein-gRNA interactions or the complex RNA fold where the extended stem meets the pseudoknot and triplex regions. More specifically, inclusion of the 2′OMe groups might have adversely affected the basal base-pairs of the gRNA extended stem and residues R49, K50, and K51 of the CasX protein. Finally, structural studies of CasX have suggested that flexibility of the gRNA is required for efficient DNA cleavage (Liu J, et al, CasX enzymes comprise a distinct family of RNA-guided genome editors. Nature 566:218-223 (2019); Tsuchida C A, et al, Chimeric CRISPR-CasX enzymes and guide RNAs for improved genome editing activity. Mol Cell 82(6): 1199-1209 (2022)). Thus, the addition of the 2′OMe groups throughout the extended stem might have enforced a more rigid A-form helical structure and prevented the needed flexibility for the gRNA for efficient cleavage. Furthermore, it is possible that the additional modifications in the scaffold stem in the v5 and v6 profiles might be detrimental to activity, though this is currently unclear given the limited comparisons between the v5 and v6 profiles.

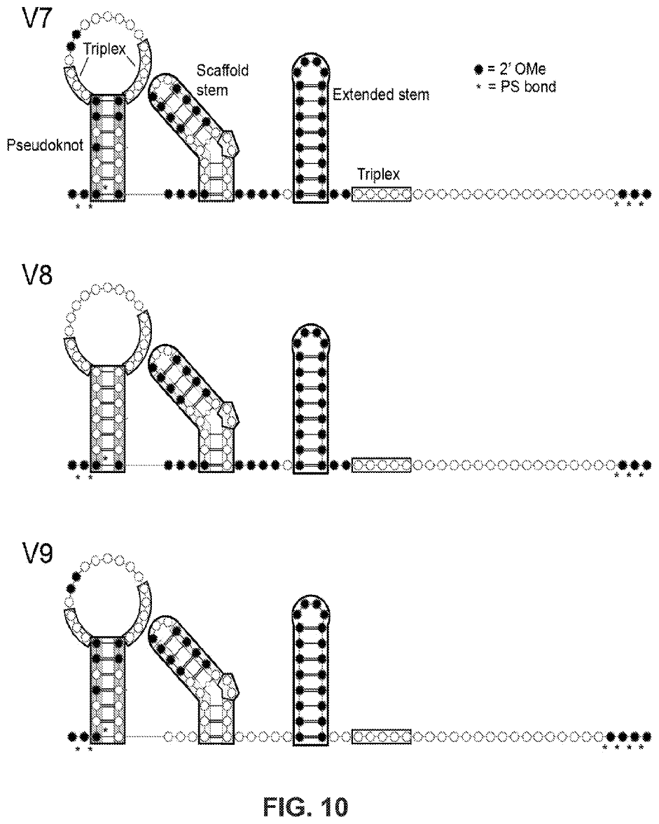

Additional modification profiles were designed with the goal of enhancing gRNA stability while mitigating the adverse effects on RNP cleavage activity. Using recently published structures of wild-type CasX from Planctomycetes (PDB codes 7WAY, 7WAZ, 7WB0, 7WB1), which has a higher homology to the CasX proteins being assessed, additional chemical modification profiles for gRNAs were designed and are illustrated in FIG. 10. These profiles illustrate the addition of 2′OMe groups and phosphorothioate bonds to a newly-designed gRNA scaffold, which is described in the ensuing sub-section. These new gRNA chemical modification profiles were designed based on the initial data demonstrating sufficient editing activity observed in Table 35 with use of the v5 gRNA that suggested that modifications to the extended stem and scaffold stem regions would not negatively impact activity. The v7 profile was designed to include 2′OMe at residues likely to be modifiable throughout the gRNA structure, which excluded the triplex region, given the dramatic negative effects of adding such modifications observed earlier with the v3 profile. More conservative profiles, v8 and v9, were also designed, as illustrated in FIG. 10. For the v8 construct, modifications were removed in the pseudoknot and triplex loop region, but were retained in the scaffold stem, extended stem, and their flanking single-stranded regions, in addition to the 5′ and 3′ termini. For the v9 profile, modifications were removed in the single-stranded regions flanking the stemloops, but were retained in the stemloops themselves, in addition to the pseudoknot, triplex loop, and 5′ and 3′ termini. The additional chemical modification profiles v7, v8, and v9 of the newly designed ERS 316 (discussed further below) were assessed in vivo at the PCSK9 locus. The results of the editing assay in vivo quantified as percent editing at the PCSK9 locus measured as indel rate as detected NGS are illustrated in FIG. 15. Despite the fact that low editing efficiency was detected overall, the data demonstrate that use of v7, v8, and v9 gRNAs resulted in lower editing levels at the PCSK9 locus compared to the indel rate achieved with use of the v1 gRNA (FIG. 15). Given the findings in FIGS. 14A-14B showing inferior editing activity attained with the v5 gRNA, it is unsurprising that v7, v8, and v9 profiles similarly demonstrated comparatively lower editing activity. As illustrated in FIG. 10, the v7, v8, and v9 profiles include modifications throughout the extended stem region, which might have interfered with RNP activity.

Comparison of gRNA Scaffold 174 and ERS 316 Using an In Vitro Cleavage Assay:

Previous work had established gRNA scaffold 235 as the top-performing scaffold across multiple delivery conditions. However, the longer length of scaffold 235 (119 bp, when using a 20 bp spacer) relative to gRNAs including scaffold 174 (109 bp, when using a 20 bp spacer) increased the difficulty of solid-phase RNA synthesis, which would result in increased manufacturing costs, decreased purity and yield, and higher rates of synthesis failures. To address these issues but retain the improved activity of using scaffold 235, a chimeric gRNA scaffold was designed primarily on the basis of the scaffold 235 sequence, but the extended stemloop of scaffold 235 was replaced with the shorter extended stemloop of scaffold 174 (FIGS. 11A-11C). The resulting chimeric scaffold, named ERS 316, was synthesized in parallel with scaffold 174 and PCSK9-targeting spacers 6.7 and 6.8, and B2M-targeting spacer 7.9 harboring the vi chemical modification profile, with 2′OMe and phosphorothioate bonds on the first and last three nucleotides of all gRNAs (see Table 30). Scaffold 174 was chosen as the comparator rather than scaffold 235 because scaffold 174 was the best previously characterized scaffold with the same length as ERS 316.

In vitro cleavage activity was assessed for gRNAs with scaffold 174 and ERS 316 and spacers 6.7 and 6.8. Cleavage assays were carried out with 20-fold excess RNP over a matching dsDNA target. Cleavage rates were quantified for all four guides, and the results are shown in Table 36. The data demonstrate that in the context of spacer 6.7, use of either scaffold 174 or ERS 316 resulted in similar cleavage rates, with ERS 316 resulting in marginally faster cleavage than that achieved with scaffold 174. In the context of spacer 6.8, the difference in cleavage activity was more pronounced: CasX RNPs using ERS 316 were able to cleave DNA nearly twice as quickly as CasX RNPs using scaffold 174 (Table 36).

Assays were also performed with equimolar amounts of RNP and DNA target over a longer time course to assess the fraction of expected RNP active for cleavage. As the CasX RNP is essentially single-turnover over the tested timescale, and the concentrations used are expected to be substantially higher than the K_{D} of the DNA-binding reaction, the amount of cleaved DNA should approximate the amount of active RNP. For either spacer 6.7 or 6.8, the active fraction of CasX RNPs incorporating ERS 316 was 25-30% higher than for CasX RNPs using scaffold 174 (Table 36). These data suggest that a higher fraction of gRNA using ERS 316 was properly folded for association with the CasX protein, or that the gRNA using ERS 316 was able to associate more strongly with the CasX protein. Compared to scaffold 174, ERS 316 harbors mutations expected to stabilize the pseudoknot and triplex structures required for proper gRNA folding. The increased stability of these motifs in particular, which were more likely to misfold than the simple hairpins found elsewhere in the gRNA structure, might result in a slightly higher fraction of the gRNAs folding into an active conformation.

TABLE 36
Parameters of cleavage activity assessed for CasX RNPs
with gRNAs containing scaffold 174 or ERS 316 with
the version 1 (v1) chemical modification profile.

| gRNA | k_{cleave} | Fraction |
|---|---|---|
| (scaffold/ERS no.-spacer) | (min⁻¹) | competence |
| 174-6.7, v1 | 0.236 | 0.194 |
| 174-6.8, v1 | 0.142 | 0.165 |
| 316-6.7, v1 | 0.264 | 0.244 |
| 316-6.8, v1 | 0.272 | 0.213 |

Comparison of gRNA Scaffold 174 and ERS 316 in a Cell-Based Assay:

An editing assessment using gRNA scaffold 174 compared to ERS 316 was performed in a cell-based assay. CasX 491 mRNA and the version 1 (v1) of PCSK9-targeting gRNAs using spacers 6.7 and 6.8 were lipofected into HepG2 cells. Treated cells were harvested 28 hours post-transfection for analysis of editing levels at the PCSK9 locus by NGS and secreted PCSK9 levels by ELISA, and the data are presented in FIG. 12. The data demonstrate that use of any of the PCSK9-targeting gRNA tested resulted in efficient editing at the PCSK9 locus and substantial reduction in PCSK9 secretion compared to the non-targeting control using the B2M-targeting gRNA. The results also show that use of ERS 316 resulted in more effective editing at the PCSK9 locus than that observed with use of scaffold 174 (˜10 percentage point increase in editing rate achieved with ERS 316 over scaffold 174). This finding is further supported by the ELISA results, such that use of ERS 316 resulted in more effective reduction of PCSK9 secretion compared to that achieved with use of scaffold 174.

Scaffold 174 and ERS 316 were also assessed in an editing assay where LNPs were formulated to co-encapsulate CasX 491 mRNA and B2M-targeting gRNA harboring either scaffold. HepG2 cells were treated with the resulting LNPs at various doses and harvested seven days post-treatment to assess editing at the B2M locus, measured as indel rate detected by NGS (FIG. 13A) and loss of surface presentation of the B2M-dependent HLA complex, as detected by flow cytometry (FIG. 13B). The results from both assays demonstrate that treatment with LNPs to deliver the B2M-targeting gRNA using ERS 316 resulted in higher editing potency at the B2M locus compared to LNPs delivering the gRNA using scaffold 174 at each dose (FIGS. 13A and 13B). Specifically, at the highest dose of 250 ng, use of ERS 316 resulted in an editing level that was nearly two-fold higher than the level attained with using scaffold 174. This substantial increase in editing efficacy when using ERS 316 versus scaffold 174, compared to the comparatively modest difference in activity observed from the in vitro cleavage assays, might be attributed to the destabilization of gRNA structure and folding during LNP formulation. The low pH conditions and association of cationic lipids during LNP formulation could adversely affect parts of the gRNA structure and result in unfolding. Consequently, it would be necessary for the gRNA to refold quickly in the cytoplasm upon delivery, both to bind the CasX protein to form the RNP and to evade RNase degradation. The stability-increasing mutations in ERS 316 compared to scaffold 174 might provide a substantial benefit in supporting proper gRNA refolding in the cytoplasm after LNP delivery, while the deliberate folding protocol carried out for the gRNA prior to biochemical experiments likely reduced the impact of these mutations.

### Example 9: CpG-Depletion of DNA Encoding the Guide RNA Scaffold Improves CasX-Mediated Editing In Vitro

Pathogen-associated molecular patterns (PAMPs), such as unmethylated CpG motifs, are small molecular motifs conserved within a class of microbes. They are recognized by toll-like receptors (TLRs) and other pattern recognition receptors in eukaryotes and often induce a non-specific immune activation. In the context of gene therapy, therapeutics containing PAMPs are often not as well-tolerated and are rapidly cleared from the patient given the strong immune response triggered, which ultimately leads to reduced therapeutic efficacy. CpG motifs are short single-stranded DNA sequences containing the dinucleotide CG. When these CpG motifs are unmethylated, they act as PAMPs and therefore stimulate the immune response. In this example, experiments were performed to deplete CpG motifs in the guide scaffold coding sequence in the context of an AAV construct encoding CasX protein 491, guide scaffold 235, and spacer 7.37 targeting the endogenous B2M locus, and test the effect of CpG-depletion in the guide scaffold on editing of the B2M locus in vitro.

### Materials and Methods

### Design of CpG-Depleted Guide Scaffolds:

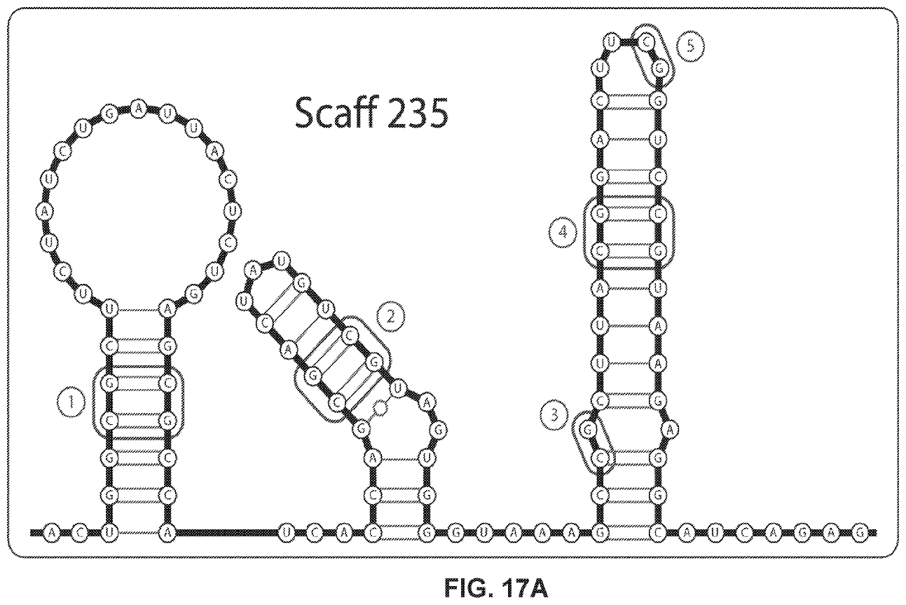

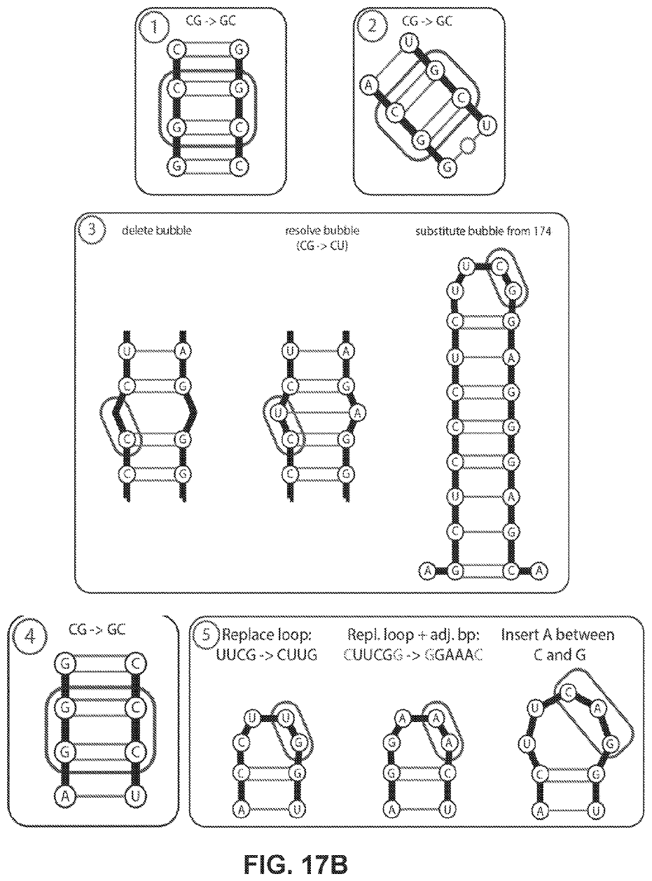

Nucleotide substitutions were rationally-designed to replace native CpG motifs within the base gRNA scaffold (gRNA scaffold 235) with the intent to preserve editing activity while reducing scaffold immunogenicity. It was believed that as many CpG-motifs as possible should be removed from the scaffold coding sequence in order to sufficiently reduce immunogenicity. Scaffold 235 contains a total of eight CpG elements; six of which are predicted to basepair and form complementary strands of a double-stranded secondary structure (see FIG. 17A). Therefore, the six basepairing CpGs forming three pairs were mutated in concert to maintain important secondary structures. This reduced the number of independent CpG-containing regions to five (three pairs and two single CpGs) to be considered independently for CpG-removal. Specifically, mutations were designed in (1) the pseudoknot stem, (2) the scaffold stem, (3) the extended stem bubble, (4) the extended step, and (5) the extended stem loop, as diagrammed in FIG. 17B and described in detail below.

In the pseudoknot stem (region 1), the CpG pair was flipped to a GpC to minimize the alteration of the base composition and sequence. Based on previous experiments involving replacing individual base pairs, it was anticipated that this mutation was not likely to be detrimental to the structure and function of the guide RNA scaffold.

Similarly, in the scaffold stem (region 2) the CpG pair was flipped to a GpC to minimize the alteration of the base composition and sequence. It was anticipated that this mutation was likely to be detrimental to the structure and function of the guide RNA scaffold because strong sequence conservation was seen in this region in previous experiments mutating individual bases or base pairs. This strong sequence conservation is likely due to the scaffold stem loop being important in interacting with the CasX protein as well as in the formation of a triplex structural element with the pseudoknot region.

In the extended stem bubble (region 3) the single CpG was removed by one of three strategies. First, the bubble was deleted by mutating CG->C. Second, the bubble was resolved to restore ideal basepairing by mutating CG->CT. Third, the entire extended stem loop was replaced with the extended stem loop of scaffold 174. Note that, by itself, the replacement of the extended stem loop with that of scaffold 174 recapitulates ERS 316, which has previously been shown to edit efficiently. There are no CpG motifs in the extended stem loop of scaffold 174. Therefore, replacing the extended stem loop with that of scaffold 174 also removes the CpG motif in the extended stem (region 4). Based on previous experiments showing the relative robustness of the extended stem to small changes, it was anticipated that mutating the extended stem bubble was moderately likely to be detrimental to the structure and function of the guide RNA scaffold.

In the extended stem (region 4), the CpG pair could not be flipped to GpC without generating additional CpG motifs. Therefore, the CpGs were changed to a GG and a complementary CC motif. Similar to region 3, based on the relative robustness of the extended stem to small changes, it was anticipated that this mutation was not likely to be detrimental to the structure and function of the guide RNA scaffold.

Finally, the extended stem loop (region 5) was mutated in one of three ways that were designed based on previous experiments examining the stability of the stem loop. In particular, several variations of the stem loop had previously been shown to have similar stability levels, and some of these variations of the stem loop do not contain CpGs. Based on these findings, first, the loop was replaced with a new loop with a CUUG sequence. Second, the loop was replaced with a new loop with a GAAA sequence. Since the GAAA loop replacement would generate a novel CpG adjacent to the loop, it was combined with a C—>G base swap and the corresponding G->C base swap on the complementary strand, ultimately resulting in a CUUCGG->GGAAAC exchange. Third, the loop was mutated by the insertion of an A to interrupt the CpG motif and thereby increase the size of the loop from 4 to 5 bases. It was anticipated that randomly mutating the extended stem loop would likely have detrimental effects on secondary structure stability and hence on editing. However, relying on previously confirmed sequences was believed to have a lower risk associated with a replacement.

To generate guide RNA scaffolds encoded by DNA with reduced CpG levels, the mutations described above were combined in various configurations. Table 37, below, summarizes combinations of the mutations that were used. In Table 37, a 0 indicates that no mutation was introduced to a given region, a 1, 2, or 3 indicates that a mutation was introduced in that region, as diagrammed in FIG. 17B, and n/a indicates not applicable. Specifically, for region 1, the pseudoknot stem, a 1 indicates that a CG->GC mutation was introduced. For region 2, the scaffold stem, a 1 indicates that a CG->GC mutation was introduced. For region 3, the extended stem bubble, a 1 indicates that the bubble was removed by the deletion of the G and A bases that form the bubble, a 2 indicates that the bubble was resolved by a CG->CU mutation that allows for basepairing between the A and U bases, and a 3 indicates that the extended stem loop was replaced with the extended step loop from guide scaffold 174. For region 4, the extended stem, a 1 indicates that a CG->GC mutation was introduced. For region 5, the extended stem loop, a 1 indicates that the loop was replaced from UUCG->CUUG, a 2 indicates that the loop was replaced along with a basepair adjacent to the loop, from CUUCGG->GGAAAC, and a 3 indicates that an A was inserted between the C and the G.

TABLE 37
Summary of mutations for CpG-reduction
and depletion in guide scaffold 235

| | | | Region 3 | | |
|---|---|---|---|---|---|
| | Region 1 | Region 2 | (Extended | Region 4 | Region 5 |
| Scaffold | (Pseudoknot | (Scaffold | stem | (Extended | (Extended |
| ID | stem) | stem) | bubble) | stem) | stem loop) |
| 320 | 1 | 0 | 0 | 1 | 0 |
| 321 | 1 | 0 | 1 | 1 | 0 |
| 322 | 1 | 0 | 2 | 1 | 0 |
| 323 | 1 | 0 | 3 | n/a | 0 |
| 324 | 1 | 0 | 1 | 1 | 1 |
| 325 | 1 | 0 | 2 | 1 | 1 |
| 326 | 1 | 0 | 3 | n/a | 1 |
| 327 | 1 | 0 | 1 | 1 | 2 |
| 328 | 1 | 0 | 2 | 1 | 2 |
| 329 | 1 | 0 | 3 | n/a | 2 |
| 330 | 1 | 0 | 1 | 1 | 3 |
| 331 | 1 | 0 | 2 | 1 | 3 |
| 332 | 1 | 0 | 3 | n/a | 3 |
| 334 | 1 | 1 | 2 | 1 | 1 |
| 335 | 1 | 1 | 3 | n/a | 1 |
| 336 | 1 | 1 | 1 | 1 | 2 |
| 337 | 1 | 1 | 2 | 1 | 2 |
| 338 | 1 | 1 | 3 | n/a | 2 |
| 339 | 1 | 1 | 1 | 1 | 3 |
| 340 | 1 | 1 | 2 | 1 | 3 |
| 341 | 1 | 1 | 3 | n/a | 3 |
| 235 | 0 | 0 | 0 | 0 | 0 |

Table 38, below, lists the DNA and RNA sequences of the designed CpG-reduced or depleted guide scaffolds.

TABLE 38
DNA and RNA sequences encoding CpG-reduced
or CpG-depleted guide RNA scaffolds

| | DNA sequence | RNA sequence |
|---|---|---|
| Scaffold ID | SEQ ID NO | SEQ ID NO |
| 320 | 535 | 160 |
| 321 | 536 | 161 |
| 322 | 537 | 162 |
| 323 | 538 | 163 |
| 324 | 539 | 164 |
| 325 | 540 | 165 |
| 326 | 541 | 166 |
| 327 | 542 | 167 |
| 328 | 543 | 168 |
| 329 | 544 | 169 |
| 330 | 545 | 170 |
| 331 | 546 | 171 |
| 332 | 547 | 172 |
| 333 | 548 | 173 |
| 334 | 549 | 174 |
| 335 | 550 | 175 |
| 336 | 551 | 176 |
| 337 | 552 | 177 |
| 338 | 553 | 178 |
| 339 | 554 | 179 |
| 340 | 555 | 180 |
| 341 | 556 | 181 |

### Generation of CpG-Depleted AAV Plasmids:

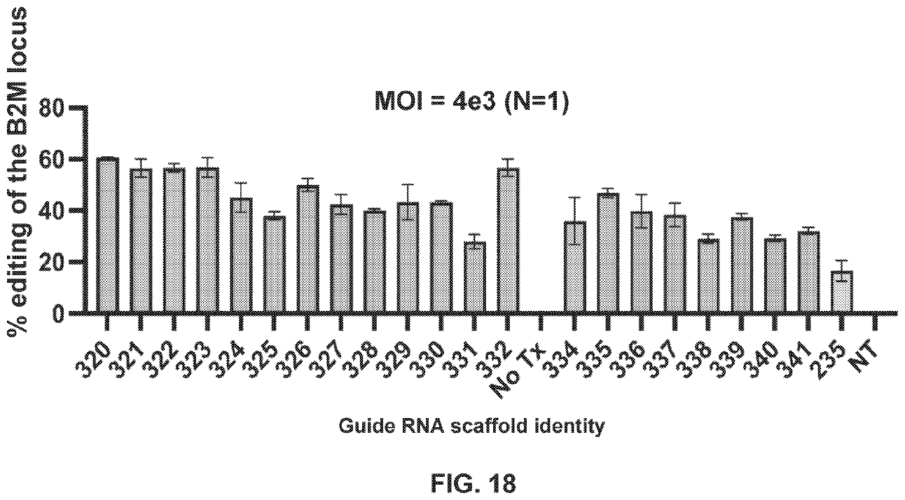

The CpG-reduced or depleted gRNA scaffolds were tested in the context of AAV vectors that were otherwise CpG-depleted, with the exception of the AAV2 ITRs. Specifically, nucleotide substitutions to replace native CpG motifs in AAV components were designed in silico based on homologous nucleotide sequences from related species for the following elements: the murine Ula snRNA (small nuclear RNA) gene promoter, the bGHpA (bovine growth hormone polyadenylation) sequence, and the human U6 promoter. The coding sequence for CasX 491 was codon-optimized for CpG depletion. All resulting sequences (Tables 38 and 39) were ordered as gene fragments with the appropriate overhangs for cloning and isothermal assembly to replace individually the corresponding elements of the existing base AAV plasmid (construct ID 183). Spacer 7.37 (GGCCGAGAUGUCUCGCUCCG; SEQ ID NO: 557), which targets the endogenous B2M gene, was used for the experiments discussed in this example. The first time that the experiment was performed (“N=1”), a sample with the non-targeting spacer 0.0 was also included as a control (CGAGACGTAATTACGTCTCG, SEQ ID NO: 558; see FIG. 18).

The resulting AAV constructs were generated using standard molecular cloning techniques. Cloned and sequence-validated plasmid constructs were midi-prepped for subsequent nucleofection and AAV vector production. The sequences of the additional components of AAV constructs, with the exception of sequences encoding the gRNAs (Table 38), are listed in Table 39.

TABLE 39
Sequences of AAV elements (5′-3′ in AAV construct)

| Element | SEQ ID NO |
|---|---|
| AAV2 5′ ITR | 559 |
| CpG-depleted U1a promoter | 560 |
| CpG-depleted cMycNLS-CasX491-cMycNLS | 561 |
| CpG-depleted bGH-polyA sequence | 562 |
| CpG-depleted U6 promoter | 563 |
| AAV2 3′ ITR | 564 |

### AAV Production:

Suspension-adapted HEK293T cells, maintained in FreeStyle 293 media, were seeded in 20-30 mL of media at 1.5E6 cells/mL on the day of transfection. Endotoxin-free pAAV plasmids with the transgene flanked by ITR repeats were co-transfected with plasmids supplying the adenoviral helper genes for replication and AAV rep/cap genome using PEI Max (Polysciences) in serum-free Opti-MEM media. Three days later, cultures were centrifuged to separate the supernatant from the cell pellet, and the AAV particles were collected, concentrated, and filtered following standard procedures.

To determine the viral genome (vg) titer, 1 μL from crude lysate viruses was digested with DNase and ProtK, followed by quantitative PCR. 5 μL of digested virus was used in a 25 μL qPCR reaction composed of IDT primetime master mix and a set of primer and 6′FAM/Zen/IBFQ probe (IDT) designed to amplify a 62 bp-fragment located in the AAV2-ITR. An AAV ITR plasmid was used as reference standards to calculate the titer (vg/mL) of viral samples.

### AAV Transduction of Induced Neurons In Vitro:

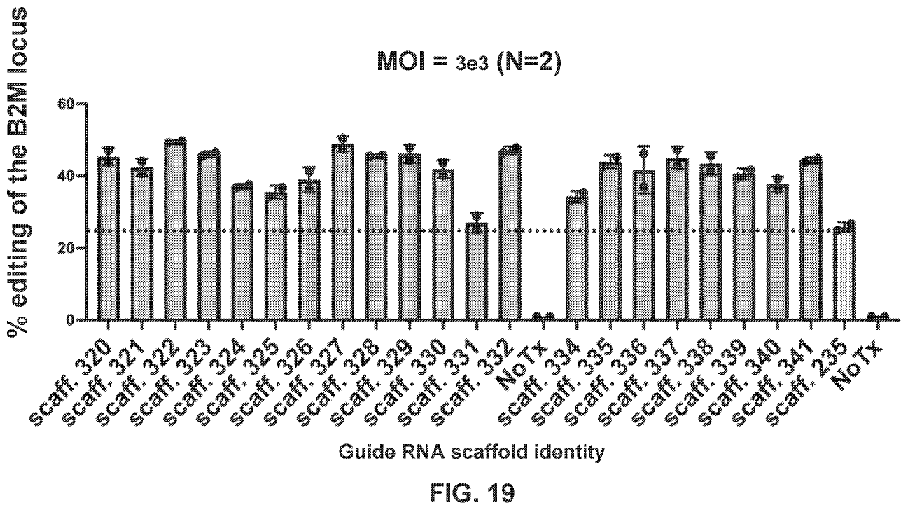

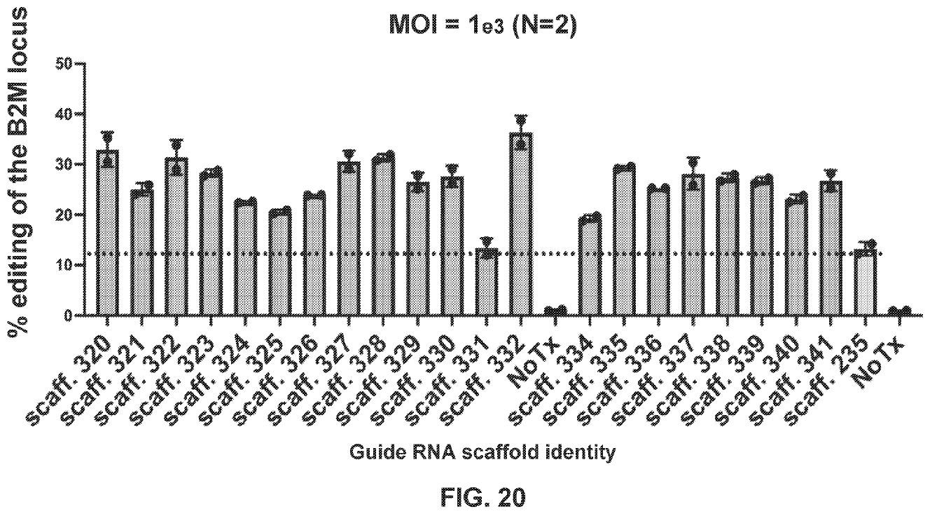

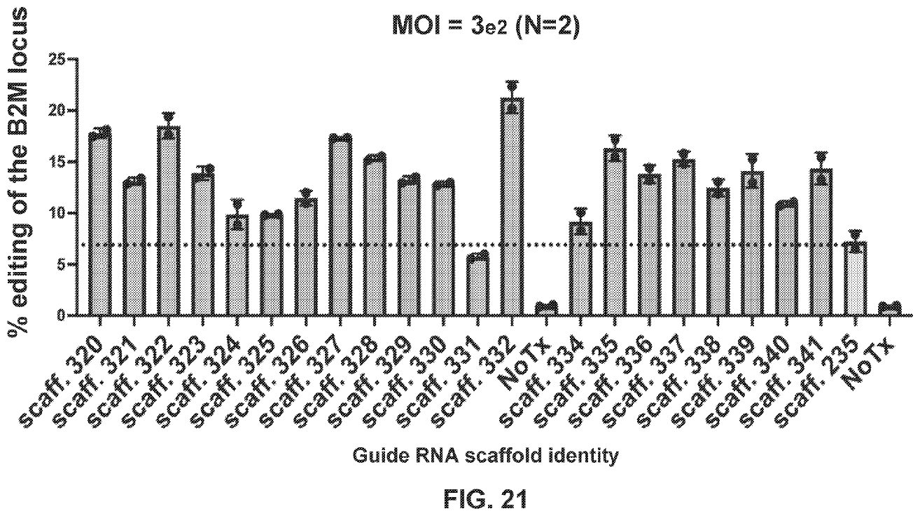

24 hours prior to transduction, 50,000 induced neurons per well were seeded on Matrigel-coated 96-well plates. AAVs expressing the CasX:gRNA system with various versions of the guide scaffold were then diluted in neuronal plating media and added to cells. The first time that the experiment was performed (“N=1”), cells were transduced at a multiplicity of infection (MOI) of 4e3 viral genomes (vg)/cell (see FIG. 18). Seven days post-plating, induced neurons were transduced with virus diluted in fresh feeding media. Eight days post-transduction, cells were lifted using lysis buffer, 4-well replicates were pooled per experimental condition, and genomic DNA (gDNA) was harvested and prepared for editing analysis at the B2M locus using next generation sequencing (NGS). The second time that the experiment was performed (“N=2”), cells were transduced at an MOI of 3e3 vg/cell, 1e3 vg/cell, or 3e2 vg/cell (see FIG. 19, FIG. 20, and FIG. 21). Seven days post-plating, induced neurons were transduced with virus diluted in fresh feeding media. Seven days post-transduction, cells were lifted using lysis buffer, 2-well replicates were pooled per experimental condition, and gDNA was harvested and prepared for editing analysis at the B2M locus using NGS. Samples that were not transduced with AAV were included as controls.

### NGS Processing and Analysis:

Genomic DNA (gDNA) from harvested cells were extracted using the Zymo Quick-DNA Miniprep Plus kit following the manufacturer's instructions. Target amplicons were formed by amplifying regions of interest from 200 ng of extracted gDNA with a set of primers specific to the human B2M gene. These gene-specific primers contained an additional sequence at the 5′ end to introduce an Illumina adapter and a 16-nucleotide unique molecule identifier. Amplified DNA products were purified with the Ampure XP DNA cleanup kit. Quality and quantification of the amplicon were assessed using a Fragment Analyzer DNA Analysis kit (Agilent, dsDNA 35-1500 bp). Amplicons were sequenced on the Illumina Miseq according to the manufacturer's instructions. Raw fastq files from sequencing were quality-controlled and processed using cutadapt v2.1, flash2 v2.2.00, and CRISPResso2 v2.0.29. Each sequence was quantified for containing an insertion or deletion (indel) relative to the reference sequence, in a window around the 3′ end of the spacer (30 bp window centered at −3 bp from 3′ end of spacer). CasX activity was quantified as the total percent of reads that contain insertions, substitutions, and/or deletions anywhere within this window for each sample.

### Results:

Mutations were introduced into the guide scaffold 235 in order to reduce the CpG content of the DNA sequence coding the guide scaffold. Surprisingly, compared to scaffold 235, all of the CpG-reduced and CpG-depleted scaffolds produced higher levels of editing in induced neurons. This was the case with two independent repeats of the experiment (with the results from the first repeat of the experiment shown in FIG. 18, and the results of the second repeat of the experiment shown in FIGS. 19-21), and across multiple MOIs (see FIGS. 19-21). The enhanced level of editing was surprising because the goal of reducing CpG content was to simply preserve editing activity while reducing immunogenicity. Instead, the mutations enhanced editing activity, rather than merely preserving it.

Notably, scaffold 320 showed a significant increase in potency over scaffold 235. Scaffold 320 includes mutations to only two regions of the scaffold; in the pseudoknot stem and the extended stem (regions 1 and 4). Further, some combinations of mutations produced worse editing than scaffold 320. However, even the CpG-reduced scaffolds that performed worse than scaffold 320, such as scaffolds 331 and 334, performed similar to or better than scaffold 235.

Based on these results, without wishing to be bound by theory, it is believed that the boost in potency seen in many of the CpG-reduced and CpG-depleted scaffolds is likely caused by one of the mutations present in all CpG-reduced scaffolds (i.e., region 1 and/or 4). Since the mutation to region 4 is not present in the scaffolds with the extended stem loop replacement (i.e., the third mutation to region 3) and these scaffolds show a similar improvement in potency over 235 as 320 did, it is believed that the beneficial effect is likely caused by the mutation in region 1 (pseudoknot stem), which is present in all of the tested scaffolds. Further experiments are performed to test the effect of the individual mutations in the pseudoknot stem (region 1) and the extended stem (region 4) separately.

Further, the N=l data as presented in FIG. 18 indicate that all the new scaffolds carrying the mutation in region 2 (scaffold stem) edited at a slightly lower level than their respective counterparts without this mutation. This suggests that mutating this position in the scaffold stem may have a small deleterious effect on editing potency. This is examined in additional experiments.

The results described here demonstrate that introducing mutations that reduced the CpG content of the DNA encoding the guide RNA scaffold resulted in improvements in gene editing relative to guide scaffold 235.

### Example 10: Additional Assessment of the Effects of Using CpG-Reduced or Depleted Guide RNAs on CasX-Mediated Editing Activity

As discussed above, unmethylated CpG motifs act as PAMPs that potently trigger undesired immune activation; therefore, nucleotide substitutions to replace native CpG motifs in the AAV constructs, including that encoding for guide scaffold 235 and ERS 316, were designed and generated. Here, experiments were performed to evaluate further the effects of using these resulting CpG-reduced or depleted gRNA scaffolds or ERSs on CasX-mediated editing activity.

### Materials and Methods

The CpG-reduced or depleted ERSs 320-341 were evaluated in three in vitro experiments described below; the sequences of ERSs 320-341 are listed in Table 38 In addition, two newly engineered gRNA ERSs, ERSs 382 and 392 (sequences listed in Table 40), were also assessed. As benchmark comparisons, scaffold 174, scaffold 235, and ERS 316 were also included for evaluation.

TABLE 40
Sequences of additional gRNA scaffolds
and ERSs tested in this example

| Scaffold or ERS ID | DNA SEQ ID NO | RNA SEQ ID NO |
|---|---|---|
| 382 | 49708 | 49725 |
| 392 | 49718 | 49735 |
| 174 | 49700 | 17 |

AAV constructs were designed and generated as previously described in Example 9. The CpG-reduced or depleted gRNA scaffolds or ERSs were tested in two different AAV backbones. Specifically, for the experiment involving lipofection of HEK293 cells as described below, scaffold 235 and ERSs 320-341 were tested in AAV vectors that were CpG-depleted, with the exception of AAV2 ITRs, as previously described in Example 9. Briefly, the CpG-depleted AAV backbone construct encoded for CpG-depleted versions of the following elements: U1A promoter, CasX 491, bGH poly(A) signal sequence, and U6 promoter. For the experiment involving AAV transduction of human induced neurons (iNs) and HEK293 cells as described below, scaffold 174, scaffold 235, ERS 316, ERS 320-341, ERS 382, and ERS 392 were tested in an AAV backbone that was not CpG-depleted (see Table 41 for sequences). Furthermore, spacer 7.37 targeting the B2M locus was used in two experiments described below involving HEK293 cells: lipofection and AAV transduction. Spacer 31.63 targeting the AAVS1 locus was used in an experiment described below involving human iNs. Table 42 below lists the AAV constructs that were tested in the context of a non-CpG-depleted AAV vector and the experimental conditions in which these constructs were assessed.

TABLE 41
Sequences encoding for a base AAV plasmid into which
gRNA scaffolds or ERSs in Table 40 were cloned

| | DNA sequence or |
|---|---|
| Component Name | SEQ ID NO |
| 5′ ITR | 487 |
| buffer sequence | 49863 |
| U1A promoter | 49864 |
| buffer sequence | 49865 |
| Kozak | GCCACC |
| start codon + c-MYC NLS | 49796 |
| linker | TCTAGA |
| CasX 515 | 49834 |
| linker | GGATCC |
| c-MYC NLS | 49841 |
| stop codon | TAA |
| buffer sequence | 49848 |
| bGH poly(A) signal sequence | 49866 |
| buffer sequence | GGTACCGT |
| U6 promoter | 49867 |
| buffer sequence | GAAACACC |
| Scaffold or ERS | See sequences listed in |
| | Tables 2, 38, 40 |
| B2M spacer (spacer 7.37) | 49849 |
| AAVS1 spacer (spacer 31.63) | 49868 |
| Non-targeting spacer (spacer 0.0) | 49745 |
| buffer sequence | 49869 |
| 3′ ITR | 488 |

TABLE 42
List of AAV constructs and scaffolds or ERSs tested in a non-
CpG-depleted AAV vector (see Table 41 for sequences) and the
experimental conditions in which these constructs were assessed

| AAV | Scaffolds | | |
|---|---|---|---|
| construct ID | or ERS | Spacer | Experimental conditions |
| 262 | 235 | 31.63 | AAV transduction in iNs |
| 263 | 328 | 31.63 | AAV transduction in iNs |
| 264 | 329 | 31.63 | AAV transduction in iNs |
| 265 | 382 | 31.63 | AAV transduction in iNs |
| 266 | 174 | 31.63 | AAV transduction in iNs |
| 267 | 335 | 31.63 | AAV transduction in iNs |
| 268 | 325 | 31.63 | AAV transduction in iNs |
| 269 | 330 | 31.63 | AAV transduction in iNs |
| 270 | 327 | 31.63 | AAV transduction in iNs |
| 271 | 334 | 31.63 | AAV transduction in iNs |
| 272 | 339 | 31.63 | AAV transduction in iNs |
| 273 | 337 | 31.63 | AAV transduction in iNs |
| 274 | 235 | Non- | AAV transduction in iNs |
| | | targeting | |
| 275 | 331 | 7.37 | AAV transduction in HEK293s |
| 276 | 335 | 7.37 | AAV transduction in HEK293s |
| 277 | 316 | 7.37 | AAV transduction in HEK293s |
| 278 | 392 | 7.37 | AAV transduction in HEK293s |
| 279 | 325 | 7.37 | AAV transduction in HEK293s |
| 280 | 334 | 7.37 | AAV transduction in HEK293s |
| 281 | 324 | 7.37 | AAV transduction in HEK293s |
| 282 | 336 | 7.37 | AAV transduction in HEK293s |
| 283 | 330 | 7.37 | AAV transduction in HEK293s |
| 284 | 320 | 7.37 | AAV transduction in HEK293s |
| 285 | 332 | 7.37 | AAV transduction in HEK293s |
| 286 | 321 | 7.37 | AAV transduction in HEK293s |
| 287 | 339 | 7.37 | AAV transduction in HEK293s |
| 288 | 235 | 7.37 | AAV transduction in HEK293s |
| 289 | 235 | Non- | AAV transduction in HEK293s |
| | | targeting | |

AAV production was performed using methods described in Example 9. For the experiment involving lipofection of HEK293 cells as described below, AAV titering was performed 9 as described in Example 9. For the two experiments involving AAV transduction of human iNs or HEK293 cells as described below, AAV titering was performed by ddPCR.

Cell-Based Assays Evaluating the Effects of Using CpG-Depleted or Reduced gRNA Scaffold or ERSs on Editing Activity:

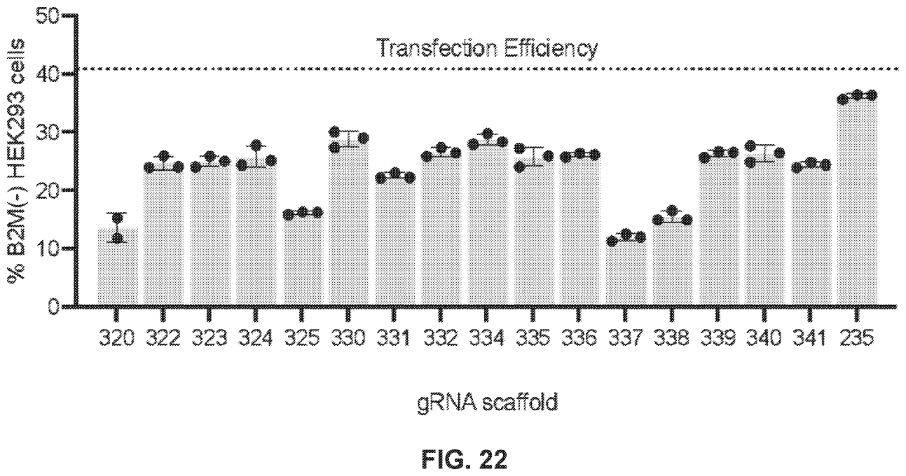

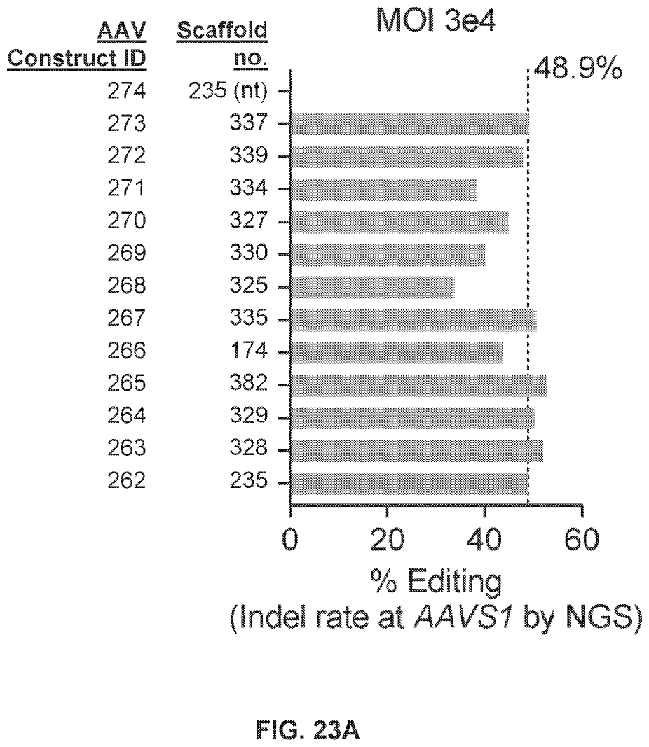

In one experiment, ˜20,000 HEK293 cells per well were seeded in 96-well plates 24 hours prior to transfection. Seeded cells were then transfected with CpG-depleted AAV plasmids containing various versions of the guide (ERS 320-341). 5 days post transfection, cells were harvested for B2M protein expression analysis via HLA immunostaining following by flow cytometry, following methods described in Example 9. A CpG-depleted AAV plasmid with scaffold 235 served as an experimental control. An AAV plasmid with a CMV promoter driving mCherry expression was used as a transfection control, and a ˜41% transfection rate was observed. The results from this experiment are shown in FIG. 22.

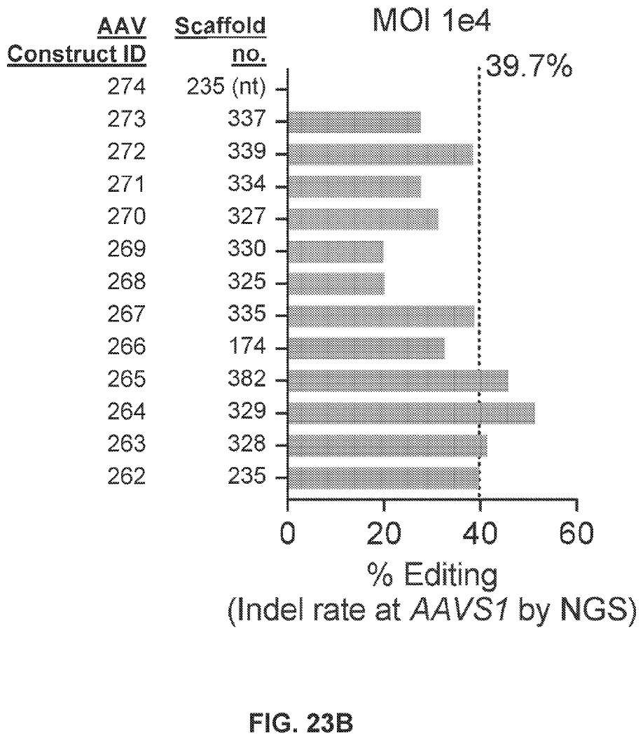

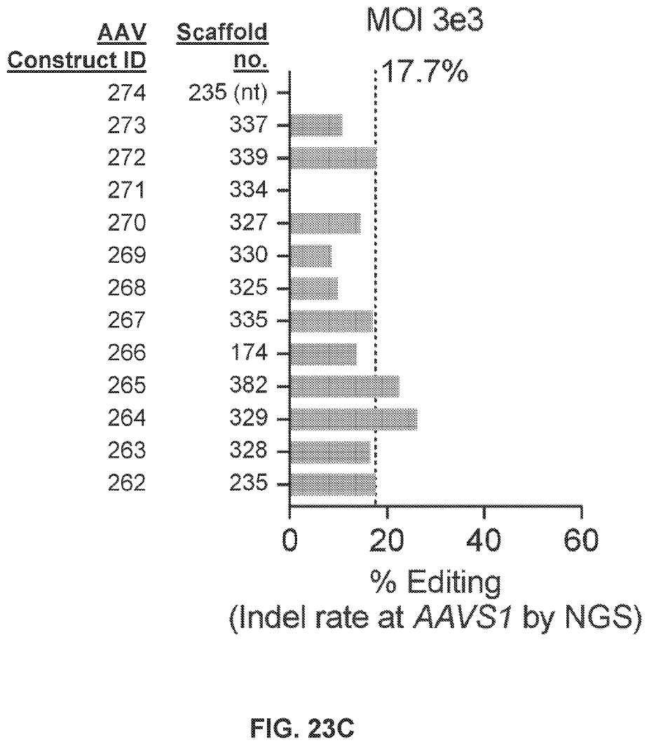

In a second experiment, ˜20,000 iNs per well were seeded on Matrigel-coated 96-well plates 7 days prior to transduction. AAVs expressing the CasX:gRNA system, containing various versions of the guide RNA (AAV construct ID #262-274; see Table 42), were diluted in neuronal plating media and added to cells 7 days post-plating. Cells were transduced at three MOIs (3E4, 1E4 or 3E3 vg/cell). 7 days post-transduction, cells were gDNA extraction for editing analysis at the AAVS1 locus using NGS following methods described in Example 9. The results from this experiment are shown in FIGS. 23A-23C.

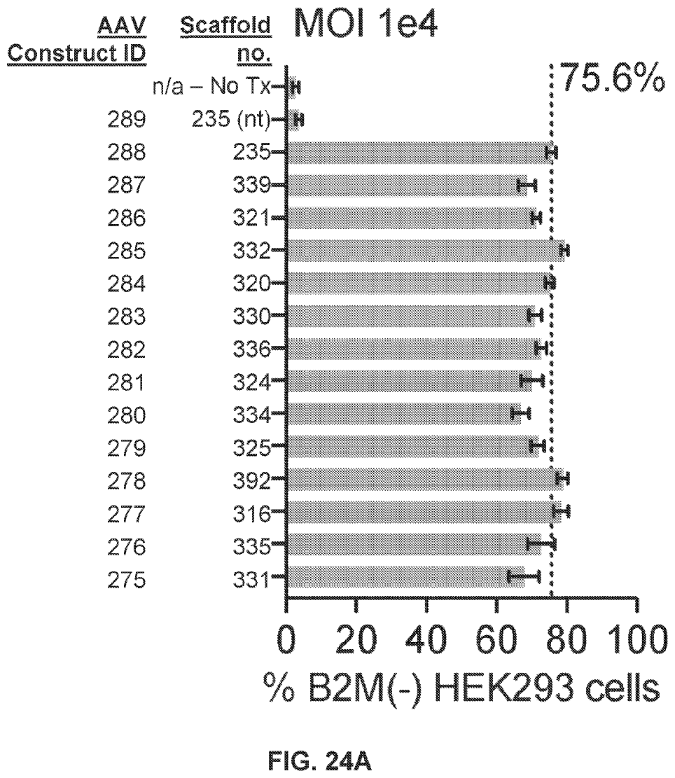

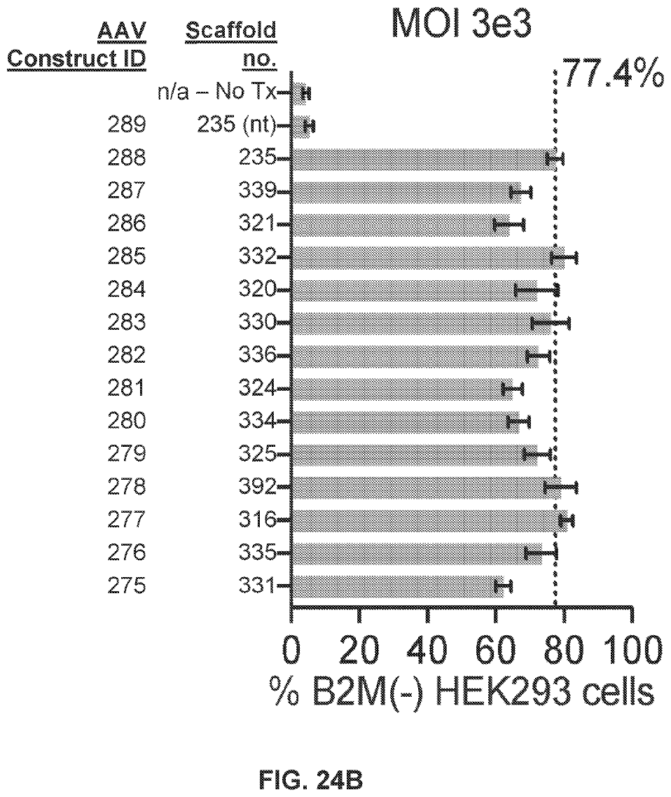

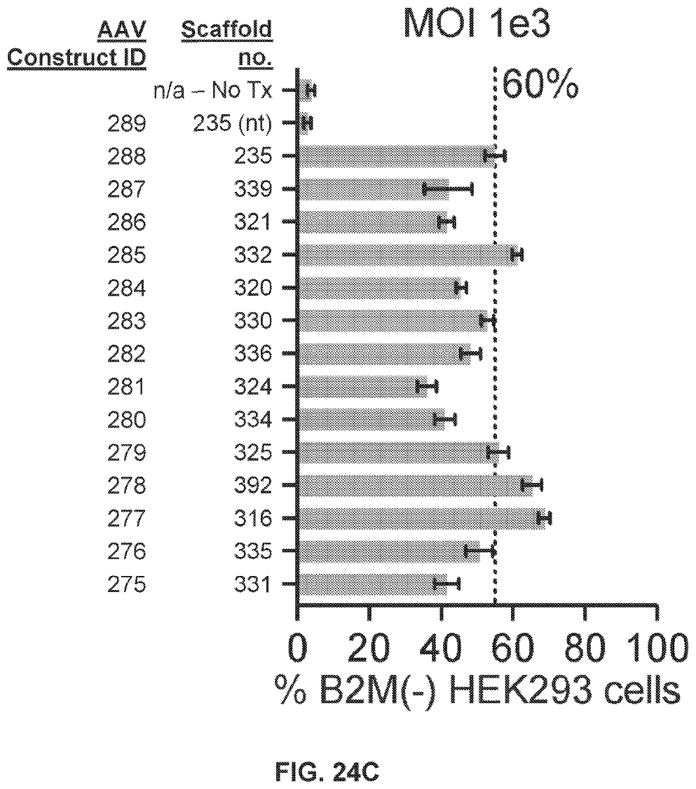

In a third experiment, ˜10,000 HEK293 cells per well were seeded in 96-well plates 24 hours prior to transfection. Seeded cells were then transduced with AAVs expressing the CasX:gRNA system, containing various versions of the guide RNA (AAV construct ID #275-289; see Table 42). Cells were transduced at three MOIs (1E4, 3E3, or 1E3 vg/cell). 5 days post-transduction, cells were harvested for B2M protein expression analysis via HLA immunostaining following by flow cytometry, following methods described in Example 9. The results from this experiment are shown in FIGS. 24A-24C.

### Results:

Experiments were performed to evaluate further the effects of using CpG-reduced or depleted ERSs on CasX-mediated editing activity. In the first experiment (N=1), HEK293 cells were lipofected with CpG-depleted AAV plasmids containing various versions of the ERS (ERS 320-341). B2M protein expression was subsequently analyzed, and the results of the assay are shown in FIG. 22. The data demonstrate that use of ERS 320-341 did not improve editing activity at the target B2M locus, since use of these ERSs produced a lower percentage of cells with B2M⁻ relative to the level achieved when using an AAV construct containing scaffold 235. These results do not recapitulate the results described in Example 9 (see FIGS. 18-21).

In the second experiment (N=1), human iNs were transduced with AAV particles expressing the CasX:gRNA system, containing various versions of the guide RNA (AAV construct ID #262-274). Editing at the AAVS1 locus was analyzed, and the results of the assay are shown in FIGS. 23A-23C. The data demonstrate that of the ERSs tested, use of ERSs 329 and 382 appeared to improve editing at the AAVS1 locus when compared to use of scaffold 235, especially at MOI of 1E4 and 3E3 vg/cell. Furthermore, the effects on editing activity were observed in a dose-dependent manner.

In the third experiment (N=1), HEK293 cells were transduced with AAV particles expressing the CasX:gRNA system, containing various versions of the guide RNA (AAV construct ID #275-289). B2M protein expression was subsequently analyzed, and the results of the assay are shown in FIGS. 24A-24C. The data demonstrate that of the guide RNAs tested, use of ERSs 316, 392 and 332 appeared to improve editing at the B2M locus when compared to use of scaffold 235 overall. Specifically, at the higher MOI of 1E4 and 3E3 vg/cell, slightly improved editing was observed with use of ERSs 316, 392, and 332 (FIGS. 24A-24B), while a stronger editing improvement was observed at the lower MOI of 1E3 vg/cell (FIG. 24C). Notably, ERS 332 and 392 both include CG>GC mutations in the pseudoknot stem (region 1; FIGS. 17A-17B), effectively reducing the overall number of CpGs when compared to scaffold 235, thereby potentially contributing to the increase in editing activity. Furthermore, ERS 316 and 332 both have a truncated extended stem when compared to scaffold 235, removing the bubble and the CG dinucleotide (region 3; FIGS. 17A-17B), thereby also potentially contributing to the observed increase in editing activity. Further experiments are performed, especially at lower MOIs, to unravel the intricacies of the effects of individual CpG mutations on editing potency.

The results from the experiments described here demonstrate that use of guide RNAs with different levels of CpG depletion can result in varying levels of editing mediated by the CasX:gRNA system, and that the resulting editing levels can vary by method of delivery (e.g., plasmid transfection vs. AAV transduction).

### Example 11: Comprehensive Sequence Determinants of Engineered Ribonucleic Acid Scaffold Activity in Complex with CasX Nuclease

The following example describes the design and evaluation of a library of engineered ribonucleic acid scaffolds (ERSs) derived from CasX guide RNA scaffolds. Experiments are performed to synthesize, screen, and analyze the ERS library for improved activity when used with a CasX-based gRNA-guided nuclease.

### Materials and Methods

### Design of ERS Library:

A library of ERSs was designed to test the effect of key mutations to individual regions in the CasX gRNA scaffold, alone and in combination. Specifically, mutations were designed to affect functional characteristics of the ERS related to improving binding to CasX to form ribonucleoproteins (RNPs), for example, through enhanced folding stability of individual domains of the ERS, enhanced folding stability of the entire ERS, increased transcriptional efficiency, or enhanced binding affinity to CasX. Further, mutations were designed to affect the function of the RNP once formed, for example, through increased cleavage activity and specificity of the CasX RNP. Finally, mutations were designed to improve manufacturability by shortening the overall length of the ERS sequence. The rationale for the design of the mutations of the library is described in detail below.

Members of the library were designed first by enumerating sequence variants of each region of the CasX gRNA scaffold that had been previously identified as conducive to improved activity. Previously, a large-scale assessment was performed to determine guide scaffold mutations that improved or worsened function. In brief, that library was composed of all single mutations in guide RNA scaffolds 174 and 175, as well as a subset of double mutations, higher-order mutations, and domain replacements with alternate synthetic sequences. Quantitative values for each mutated scaffold's effect on function were obtained from this screen. Several different selection criteria were applied to identify top mutations to incorporate into the ERS library described herein and test in subsequent rounds of experimentation. These mutations could be single mutations, double mutations, or entire domain swaps. The criteria were applied to be able to obtain mutations that could be acting on different functional “levers” that would affect activity of the ERS-nuclease complex and came from multiple different regions of the ERS, and so could conceivably be stacked together to obtain additive functional effects without the mutations negatively interacting. In brief, criteria as outlined below were used to identify mutations.

(a) Single mutations that had consistently higher activity in the context of both guide scaffold 174 and guide scaffold 175, based on enrichment scores greater than the reference scaffold for at least one scaffold context, and enrichment scores greater than 0 for the other scaffold context.

(b) A subset of single mutations that had high activity in the context of both guide scaffold 174 and guide scaffold 175 based on enrichment score greater than 0 for both scaffold, and were at novel positions compared to the previous set (a), to diversify the mutations to assess.

(c) Double mutations with high activity that were composed of single mutations that are individually low activity. This criterion was chosen to obtain more diversified sequences (e.g., with multiple mutations) that would not be achieved by additive stacking of high-activity single mutations. These mutations comprised structurally interacting residues. Double mutations with high activity were defined as positive enrichment in the context of both guide scaffold 174 and guide scaffold 175 or had enrichment greater than at least one of guide scaffold 174 or guide scaffold 175; single mutations with low activity were defined as those with no positive enrichment in either guide scaffold 174 or guide scaffold 175.

(d) Double mutations with higher activity than the reference scaffolds in the context of guide scaffold 174 and guide scaffold 175, or just compared to guide scaffold 175 scaffold.

(e) Single mutations that were present in the double mutations in set (d). These variants were included in order to serve as important reference points for the double mutation scaffolds.

(f) The top ˜10 extended stem replacements or extended stem mutations that were enriched in the context of either guide scaffold 174 or guide scaffold 175.

(g) The top 5 pseudoknot stems that were enriched in the context of either guide scaffold 174 or guide scaffold 175.

Alternatively, additional members of the ERS library were rationally designed with mutations to improve functional characteristics of the ERS. These mutations were added to the library despite not being found in the large-scale assessment described above. First, truncations of the 5′ end were introduced into the library. Transcription from a U6 promoter starts at a defined register for A and G residues, but not C or T residues; thus starting with A or G ensures the integrity of the transcript sequence (see Gao, Z. et al., Transcription. 2017; 8(5): 275-287). Thus, for this mutation, the 5′ A and C were deleted, but the next residue was changed from a T to a G for transcriptional integrity, while the predicted base-paired base to that T at position 33 was changed from an A to a C to compensate for the disrupted base pair. For this mutation, the T-A pair at positions 3 and 33 were replaced with a G-C at positions 3 and 33, with the A and C at positions 1 and 2 deleted.

Further, triplex stabilizing mutations were introduced into the ERS library. The large-scale assessment described above was not able to test triplex variants due to limitations in the cloning methods, but an independent line of inquiry has determined that guide scaffolds 214 and 215, each of which contains three mutated residues in one position of the triplex, had improved activity when introduced into guide scaffold 174 and 175 (data not shown). The three mutations in scaffold 215 ultimately became incorporated into guide scaffold 235 and ERS 316. Therefore, triplex stabilizing mutations were introduced into the library to probe whether adding additional positions would improve behavior, or whether reverting any of these mutations is very deleterious, in order to better understand the effect of this triple mutation on structure and function.

Finally, truncated extended stems were introduced into the ERS library. These sequences introduced sequential deletions in the extended stem sequence of the scaffold, as well as some loop deletions, and base pair swaps, that were intended to confer additional stability on the extended stem formation while truncating the stem. The goal of truncating the stem was to generate a shorter overall ERS for improved manufacturability.

Taken together, based on the above analyses and rational designs, the regions and domains that were modified in the ERS library were as follows (regions as summarized in Table 43 and diagrammed in FIG. 25):
(1) 5′ end variants (N=15), which are hypothesized to increase transcriptional efficiency by varying the 5′ end, increasing manufacturability by shortening the 5′ end, and/or increase folding stability of the variant gRNA structure;
(2) pseudoknot stem variants (N=49), which are hypothesized to increase folding stability of the pseudoknot stem and thus increase folding stability of the variant gRNA structure;
(3) triplex loop variants (N=19), which may increase folding stability of the variant gRNA structure, increase binding affinity to the nuclease, and/or increase manufacturability by shortening the triplex loop sequence;
(4) triplex variants (including adjacent sequence between the extended stem and the start of annotated triplex; N=19), which may increase folding stability of the variant gRNA structure;
(5) scaffold stem variants (including adjacent sequences from the end of pseudoknot and start of extended stem; N=27), which may increase folding stability of the scaffold stem and, thus, increase folding stability of the variant gRNA structure, increase binding affinity to the nuclease, and/or affect the function of the RNP once formed, e.g., through increased cleavage activity and specificity of the CasX RNP; and
(6) extended stem variants (N=33), which may increase folding stability of the extended stem and, thus, increase folding stability of the variant gRNA structure, and/or increase manufacturability through substantial truncations in the ERS length.

TABLE 43
Summary of positions of mutations in ERS library relative to ERS 316

| Region of mutation | RNA sequence in ERS 316 | SEQ ID NO |
|---|---|---|
| 5′ end | AC | N/A |
| Pseudoknot stem | UGGCGCU_AGCGCCA | 565 |
| Triplex loop | AUCUGAUUA | 566 |
| Triplex (including adjacent sequence | UCU_CUCUG_AUCAGAG | 567 |
| between the extended stem and the start | | |
| of annotated triplex) | | |
| Scaffold stem (including adjacent | UCACCAGCGACUAUGUCGUAGUG | 568 |
| sequences from the end of pseudoknot | GGUAAA | |
| and start of extended stem) | | |
| Extended stem | GCUCCCUCUUCGGAGGGAGC | 569 |

The individual region mutations that were identified for inclusion in the library are presented in Table 44. Note that in the tables, there can be multiple mutated bases in a given region, but that the mutations in each row of the table are considered an “individual mutation” for purposes of assembling the library.

TABLE 44
Mutations to scaffold 221 (RNA sequence)

| | Region of |
|---|---|
| Mutation* | mutation |
| 0.—.A | 5′ end |
| 0.A.G | 5′ end |
| 1.C.— | 5′ end |
| 1.—.U | 5′ end |
| 2.U.— | 5′ end |
| 3.—.A | 5′ end |
| 0.A.U;1.C.A | 5′ end |
| 1.C.—;2.U.— | 5′ end |
| 1.C.—;5.C.U | 5′ end |
| 0.A.U | 5′ end |
| 1.C.A | 5′ end |
| 1.C.G | 5′ end |
| 2.U.A | 5′ end |
| 2.U.G | 5′ end |
| 0.A.—;1.C.—;2.U.G;32.A.C | 5′ end |
| 12.—.A | Triplex loop |
| 13.U.A | Triplex loop |
| 13.U.G | Triplex loop |
| 13.U.— | Triplex loop |
| 14.C.A | Triplex loop |
| 14.C.U | Triplex loop |
| 14.C.G | Triplex loop |
| 15.—.U | Triplex loop |
| 18.—.U | Triplex loop |
| 17.—.U | Triplex loop |
| 17.—.A | Triplex loop |
| 17.A.G | Triplex loop |
| 17.A.U | Triplex loop |
| 20.A.G | Triplex loop |
| 11.—.U;11.—.A | Triplex loop |
| 14.—.U;14.—.A | Triplex loop |
| 13.U.C | Triplex loop |
| 15.U.A | Triplex loop |
| 15.U.G | Triplex loop |
| 25.G.C;93.C.G | Triplex |
| 9.UCU.UUU | Triplex |
| 21.CUCUG.CUUUG | Triplex |
| 93.CAGAG.CAAAG | Triplex |
| 9.UCU.UUU;21.CUCUG.CUUUG | Triplex |
| 9.UCU.UUU;93.CAGAG.CAAAG | Triplex |
| 21.CUCUG.CUUUG;93.CAGAG.CAAAG | Triplex |
| 9.UCU.UGU | Triplex |
| 9.UCU.UCC;21.CUCUG.CCCUG;93.CAGAG.CAGGG | Triplex |
| 9.UCU.CCU;21.CUCUG.CUCCG;93.CAGAG.CGGAG | Triplex |
| 9.UCU.UGG;21.CUCUG.CCCUG;93.CAGAG.CAGGG | Triplex |
| 9.UCU.GGU;21.CUCUG.CUCCG;93.CAGAG.CGGAG | Triplex |
| 9.UCU.CCC;21.CUCUG.CCCCG;93.CAGAG.CGGGG | Triplex |
| 9.UCU.GGG;21.CUCUG.CCCCG;93.CAGAG.CGGGG | Triplex |
| 21.CUCUG.GUCUC;93.CAGAG.GAGAC | Triplex |
| 21.CUCUG.GUCUG;93.CAGAG.CAGAC | Triplex |
| 21.CUCUG.CUCUC;93.CAGAG.CAGAC | Triplex |
| 91.A.C | Triplex |
| 91.A.G | Triplex |
| 6.A.U | Pseudoknot |
| 6.A.G | Pseudoknot |
| 6.A.G;28.A.C | Pseudoknot |
| 6.A.G;28.A.U | Pseudoknot |
| 6.A.G;28.—.C | Pseudoknot |
| 28.—.U;6.A.G | Pseudoknot |
| 26.A.U;6.A.G | Pseudoknot |
| 28.A.G;6.A.G | Pseudoknot |
| 32.A.U;6.A.G | Pseudoknot |
| 5.C.G;29.G.C;6.A.G | Pseudoknot |
| 6.A.U;28.A.C | Pseudoknot |
| 6.A.C;28.A.G | Pseudoknot |
| 6.A.C;28.A.U | Pseudoknot |
| 6.A.U;28.A.G | Pseudoknot |
| 28.—.C;28.A.U;6.A.G | Pseudoknot |
| 28.A.C;29.G.A;6.A.G | Pseudoknot |
| 28.A.U;32.A.G;6.A.G | Pseudoknot |
| 28.A.C | Pseudoknot |
| 28.—.U | Pseudoknot |
| 28.A.U | Pseudoknot |
| 6.A.C;28.—.G | Pseudoknot |
| 6.A.U;28.A.U | Pseudoknot |
| 6.A.C;27.—.G | Pseudoknot |
| 7.C.G;28.A.C;6.A.G | Pseudoknot |
| 5.CA.GC;28.AG.GC | Pseudoknot |
| 6.A.C | Pseudoknot |
| 5.C.U;6.A.G | Pseudoknot |
| 7.C.G;6.A.G | Pseudoknot |
| 26.A.G;6.A.G | Pseudoknot |
| 27.—.G;6.A.G | Pseudoknot |
| 27.G.U;6.A.G | Pseudoknot |
| 28.—.C;6.A.G | Pseudoknot |
| 28.—.U;6.A.G | Pseudoknot |
| 29.G.A;6.A.G | Pseudoknot |
| 32.A.G;6.A.G | Pseudoknot |
| 26.A.G;28.A.C;6.A.G | Pseudoknot |
| 26.A.G;28.A.C;6.A.G | Pseudoknot |
| 26.A.G;28.A.C;6.A.G | Pseudoknot |
| 2.UGGCAC.CUGUAG;27.GAGCCA.CUACAG | Pseudoknot |
| 2.UGGCAC.CAGCAA;27.GAGCCA.UUGCUG | Pseudoknot |
| 2.UGGCAC.CGAGAC;27.GAGCCA.GGCUCG | Pseudoknot |
| 2.UGGCAC.ACUGGU;27.GAGCCA.ACCAGU | Pseudoknot |
| 2.UGGCAC.AGGCCG;27.GAGCCA.CGGCCU | Pseudoknot |
| 2.UGGCAC.ACACGG;27.GAGCCA.CCGUGU | Pseudoknot |
| 2.UGGCAC.GGGACU;27.GAGCCA.AGUCCC | Pseudoknot |
| 2.UGGCAC.GGGCAA;27.GAGCCA.UUGCCC | Pseudoknot |
| 2.UGGCAC.GAGCAC;27.GAGCCA.GGGCUC | Pseudoknot |
| 2.UGGCAC.UGUGCG;27.GAGCCA.CGCACA | Pseudoknot |
| 2.UGGCAC.AGGUCC;27.GAGCCA.GGACCU | Pseudoknot |
| 2.UGGCAC.GAGGUG;27.GAGCCA.CACCUC | Pseudoknot |
| 44.U.G | Scaffold stem |
| 53.—.G | Scaffold stem |
| 53.—.U | Scaffold stem |
| 35.A.U | Scaffold stem |
| 35.A.C | Scaffold stem |
| 43.C.U | Scaffold stem |
| 52.A.G | Scaffold stem |
| 56.—.U | Scaffold stem |
| 56.—.A | Scaffold stem |
| 33.U.A | Scaffold stem |
| 34.C.G;56.G.C | Scaffold stem |
| 34.C.G;57.—.C | Scaffold stem |
| 35.A.G;56.—.C | Scaffold stem |
| 38.A.U;53.U.A | Scaffold stem |
| 40.C.A,50.G.U | Scaffold stem |
| 40.C.G;50.G.C | Scaffold stem |
| 41.G.C;49.C.G | Scaffold stem |
| 41.G.U;49.C.A | Scaffold stem |
| 58.A.—;59.A.— | Scaffold stem |
| 40.CG.GC;49.CG.GC;53.—.U | Scaffold stem |
| 33.U.G | Scaffold stem |
| 34.C.G | Scaffold stem |
| 35.A.G | Scaffold stem |
| 45.A.C | Scaffold stem |
| 56.—.C | Scaffold stem |
| 57.—.C | Scaffold stem |
| 58.A.G | Scaffold stem |
| 59.A.G | Scaffold stem |
| 61.GCCGCUUACGGACUUCGGUCCGUAAGAGGC.GGUGACGGUCUUC | Extended stem |
| GGACCGUCACC | |
| 61.GCCGCUUACGGACUUCGGUCCGUAAGAGGC.UCGUAAGAACUUC | Extended stem |
| GGUUCUUACGA | |
| 61.GCCGCUUACGGACUUCGGUCCGUAAGAGGC.GUGCGCCCGCUUC | Extended stem |
| GGCGGGCGCAC | |
| 61.GCCGCUUACGGACUUCGGUCCGUAAGAGGC.UAAUGAAAACUUC | Extended stem |
| GGUUUUCAUUA | |
| 61.GCCGCUUACGGACUUCGGUCCGUAAGAGGC.UGGAAGAUGCUUC | Extended stem |
| GGCAUCUUCCA | |
| 61.GCCGCUUACGGACUUCGGUCCGUAAGAGGC.GCGGCAGAUCUGA | Extended stem |
| GCCUCCGAGCUCUCUGCCGC | |
| 61.GCCGCUUACGGACUUCGGUCCGUAAGAGGC.GCUCCCUCACAGG | Extended stem |
| GAUGUGAGGGAGC | |
| 61.GCCGCUUACGGACUUCGGUCCGUAAGAGGC.GCUCCCUCAGCUG | Extended stem |
| CAGUGAGGGAGC | |
| 61.GCCGCUUACGGACUUCGGUCCGUAAGAGGC.GCGCUUACGGACU | Extended stem |
| UCGGUCCGUAAGAAGC | |
| 61.GCCGCUUACGGACUUCGGUCCGUAAGAGGC.GCGCUUACGGAAU | Extended stem |
| UCGUGUCCGUAAGAAGC | |
| 61.GCCGCUUACGGACUUCGGUCCGUAAGAGGC.GCGCUUUCGGACU | Extended stem |
| UCGGUCCGGAAGAAGC | |
| 61.GCCGCUUACGGACUUCGGUCCGUAAGAGGC.GCGCCUUACGGAC | Extended stem |
| UUCGGUCCGUAAGGAGC | |
| 61.GCCGCUUACGGACUUCGGUCCGUAAGAGGC.GCGCUUACGGACU | Extended stem |
| UCGCGGUCCGUAAGAAGC | |
| 61.GCCGCUUACGGACUUCGGUCCGUAAGAGGC.GCGCUUACGGACU | Extended stem |
| UCGGUCCGUAAGUCGC | |
| 61.GCCGCUUACGGACUUCGGUCCGUAAGAGGC.GCGCUUACGGAGU | Extended stem |
| UCGAUCCGUAAGAAGC | |
| 61.GCCGCUUACGGACUUCGGUCCGUAAGAGGC.GCGGUUACGGACU | Extended stem |
| UCGGUCCGUAAGACGC | |
| 61.GCCGCUUACGGACUUCGGUCCGUAAGAGGC.GCUCCCUCUUCGG | Extended stem |
| AGGGAGC | |
| 61.GCCGCUUACGGACUUCGGUCCGUAAGAGGC.GCUCCCUCUUCGG | Extended stem |
| AGGAGGC | |
| 61.GCCGCUUACGGACUUCGGUCCGUAAGAGGC.GCUCCCUCUUCGG | Extended stem |
| AGAGUGC | |
| 61.GCCGCUUACGGACUUCGGUCCGUAAGAGGC.GCUCCCUAUUCGG | Extended stem |
| AUGGAGC | |
| 61.GCCGCUUACGGACUUCGGUCCGUAAGAGGC.GCUCCCUCUUCGG | Extended stem |
| AGGGAACGC | |
| 61.GCCGCUUACGGACUUCGGUCCGUAAGAGGC.GCUCCCUCAUAGG | Extended stem |
| AGGGAGC | |
| 61.GCCGCUUACGGACUUCGGUCCGUAAGAGGC.GUCUCCCUCUUCG | Extended stem |
| GAGGGAGGC | |
| 61.GCCGCUUACGGACUUCGGUCCGUAAGAGGC.GCUCCUCUUCGGA | Extended stem |
| GGAGC | |
| 61.GCCGCUUACGGACUUCGGUCCGUAAGAGGC.GCUCUCUUCGGAG | Extended stem |
| AGC | |
| 61.GCCGCUUACGGACUUCGGUCCGUAAGAGGC.GCUCCCUUCGGGG | Extended stem |
| AGC | |
| 61.GCCGCUUACGGACUUCGGUCCGUAAGAGGC.GCUCCGUUCGCGG | Extended stem |
| AGC | |
| 61.GCCGCUUACGGACUUCGGUCCGUAAGAGGC.GCUGCUUCGGCAG | Extended stem |
| C | |
| 61.GCCGCUUACGGACUUCGGUCCGUAAGAGGC.GCUGCAGC | Extended stem |
| 61.GCCGCUUACGGACUUCGGUCCGUAAGAGGC.GCUAGC | Extended stem |
| 61.GCCGCUUACGGACUUCGGUCCGUAAGAGGC.CUCCCUCUUCGGA | Extended stem |
| GGGAG | |
| 61.GCCGCUUACGGACUUCGGUCCGUAAGAGGC.GCGCCCUCUUCGG | Extended stem |
| AGGGAGC | |
| 61.GCCGCUUACGGACUUCGGUCCGUAAGAGGC.GGUCCCUCUUCGG | Extended stem |
| AGGGAAC | |
| 64.G.—;87.A.— | Extended stem |
| 64.G.U | Extended stem |
| 69.C.G;82.G.C | Extended stem |
| 61.GCCGCUUACGGACUUCGGUCCGUAAGAGGC.GCUCCCUCUUCAG | Extended stem |
| GAGGGAGC | |
| 61.GCCGCUUACGGACUUCGGUCCGUAAGAGGC.GCUCCCUGGAAAC | Extended stem |
| AGGGAGC | |

*positions are numbered relative to the 5′ end of guide scaffold 221, with “0” being the 5′ terminus. Each mutation is indicated by its position, reference sequence, and alt sequence, separated by ‘.’ (e.g., 1.C.A is nucleotide position 1, reference sequence nucleotide C, alternate sequence nucleotide A). Position indexing starts at 0 such that the first base in scaffold 221 is 0.A. Insertions are indicated with a ‘—’ in the reference sequence (first position), and deletions with a ‘—’ in the alt sequence (second position). Multiple individual mutations are semi-colon separated.

The individual mutations of Table 44 were then introduced into the comparable relative positions of the ERS 316 scaffold (taking into account the differences in the extended stem positions and the individual differences between scaffolds 221 and 316 in the other regions as shown in Table 45), and Table 46 lists the DNA and RNA sequences of the ERS.

TABLE 45
Additional sequence changes applied to ERS
to convert parental scaffold 221 to ERS 316

| Mutation* | Region |
|---|---|
| 6.A.G;28.A.C | Pseudoknot stem |
| 53.—.G | Scaffold stem |
| 61.GCCGCTTACGGACTTCGGT | Extended stem |
| CCGTAAGAGGC.GCTCCCTCTT | |
| CGGAGGGAGC | |

*positions are numbered relative to the 5′ end of guide scaffold 221, with “0” being the 5′ terminus. Each mutation is indicated by its position, reference sequence, and alt sequence, separated by ‘.’ (e.g., 1.C.A is nucleotide position 1, reference sequence nucleotide C, alternate sequence nucleotide A). Position indexing starts at 0 such that the first base in scaffold 221 is 0.A. Insertions are indicated with a ‘—’ in the reference sequence (first position), and deletions with a ‘—’ in the alt sequence (second position). Multiple individual mutations are semi-colon separated.

TABLE 46
DNA and RNA sequences of ERSs with individual mutations

| Region of | DNA sequence | RNA sequence |
|---|---|---|
| mutation | SEQ ID NO | SEQ ID NO |
| 5′ end | 570 | 739 |
| 5′ end | 571 | 740 |
| 5′ end | 572 | 741 |
| 5′ end | 573 | 742 |
| 5′ end | 574 | 743 |
| 5′ end | 575 | 744 |
| 5′ end | 576 | 745 |
| 5′ end | 577 | 746 |
| 5′ end | 578 | 747 |
| 5′ end | 579 | 748 |
| 5′ end | 580 | 749 |
| 5′ end | 581 | 750 |
| 5′ end | 582 | 751 |
| 5′ end | 583 | 752 |
| 5′ end | 584 | 753 |
| Triplex loop | 585 | 754 |
| Triplex loop | 586 | 755 |
| Triplex loop | 587 | 756 |
| Triplex loop | 588 | 757 |
| Triplex loop | 589 | 758 |
| Triplex loop | 590 | 759 |
| Triplex loop | 591 | 760 |
| Triplex loop | 592 | 761 |
| Triplex loop | 593 | 762 |
| Triplex loop | 594 | 763 |
| Triplex loop | 595 | 764 |
| Triplex loop | 596 | 765 |
| Triplex loop | 597 | 766 |
| Triplex loop | 598 | 767 |
| Triplex loop | 599 | 768 |
| Triplex loop | 600 | 769 |
| Triplex loop | 601 | 770 |
| Triplex loop | 602 | 771 |
| Triplex loop | 603 | 772 |
| Triplex | 604 | 773 |
| Triplex | 605 | 774 |
| Triplex | 606 | 775 |
| Triplex | 607 | 776 |
| Triplex | 608 | 777 |
| Triplex | 609 | 778 |
| Triplex | 610 | 779 |
| Triplex | 611 | 780 |
| Triplex | 612 | 781 |
| Triplex | 613 | 782 |
| Triplex | 614 | 783 |
| Triplex | 615 | 784 |
| Triplex | 616 | 785 |
| Triplex | 617 | 786 |
| Triplex | 618 | 787 |
| Triplex | 619 | 788 |
| Triplex | 620 | 789 |
| Triplex | 621 | 790 |
| Triplex | 622 | 791 |
| Pseudoknot | 623 | 792 |
| Pseudoknot | 624 | 793 |
| Pseudoknot | 625 | 794 |
| Pseudoknot | 626 | 795 |
| Pseudoknot | 627 | 796 |
| Pseudoknot | 628 | 797 |
| Pseudoknot | 629 | 798 |
| Pseudoknot | 630 | 799 |
| Pseudoknot | 631 | 800 |
| Pseudoknot | 632 | 801 |
| Pseudoknot | 633 | 802 |
| Pseudoknot | 634 | 803 |
| Pseudoknot | 635 | 804 |
| Pseudoknot | 636 | 805 |
| Pseudoknot | 637 | 806 |
| Pseudoknot | 638 | 807 |
| Pseudoknot | 639 | 808 |
| Pseudoknot | 640 | 809 |
| Pseudoknot | 641 | 810 |
| Pseudoknot | 642 | 811 |
| Pseudoknot | 643 | 812 |
| Pseudoknot | 644 | 813 |
| Pseudoknot | 645 | 814 |
| Pseudoknot | 646 | 815 |
| Pseudoknot | 647 | 816 |
| Pseudoknot | 648 | 817 |
| Pseudoknot | 649 | 818 |
| Pseudoknot | 650 | 819 |
| Pseudoknot | 651 | 820 |
| Pseudoknot | 652 | 821 |
| Pseudoknot | 653 | 822 |
| Pseudoknot | 654 | 823 |
| Pseudoknot | 655 | 824 |
| Pseudoknot | 656 | 825 |
| Pseudoknot | 657 | 826 |
| Pseudoknot | 658 | 827 |
| Pseudoknot | 659 | 828 |
| Pseudoknot | 660 | 829 |
| Pseudoknot | 661 | 830 |
| Pseudoknot | 662 | 831 |
| Pseudoknot | 663 | 832 |
| Pseudoknot | 664 | 833 |
| Pseudoknot | 665 | 834 |
| Pseudoknot | 666 | 835 |
| Pseudoknot | 667 | 836 |
| Pseudoknot | 668 | 837 |
| Pseudoknot | 669 | 838 |
| Pseudoknot | 670 | 839 |
| Pseudoknot | 671 | 840 |
| Pseudoknot | 672 | 841 |
| Scaffold stem | 673 | 842 |
| Scaffold stem | 674 | 843 |
| Scaffold stem | 675 | 844 |
| Scaffold stem | 676 | 845 |
| Scaffold stem | 677 | 846 |
| Scaffold stem | 678 | 847 |
| Scaffold stem | 679 | 848 |
| Scaffold stem | 680 | 849 |
| Scaffold stem | 681 | 850 |
| Scaffold stem | 682 | 851 |
| Scaffold stem | 683 | 852 |
| Scaffold stem | 684 | 853 |
| Scaffold stem | 685 | 854 |
| Scaffold stem | 686 | 855 |
| Scaffold stem | 687 | 856 |
| Scaffold stem | 688 | 857 |
| Scaffold stem | 689 | 858 |
| Scaffold stem | 690 | 859 |
| Scaffold stem | 691 | 860 |
| Scaffold stem | 692 | 861 |
| Scaffold stem | 693 | 862 |
| Scaffold stem | 694 | 863 |
| Scaffold stem | 695 | 864 |
| Scaffold stem | 696 | 865 |
| Scaffold stem | 697 | 866 |
| Scaffold stem | 698 | 867 |
| Scaffold stem | 699 | 868 |
| Scaffold stem | 700 | 869 |
| Extended stem | 701 | 870 |
| Extended stem | 702 | 871 |
| Extended stem | 703 | 872 |
| Extended stem | 704 | 873 |
| Extended stem | 705 | 874 |
| Extended stem | 706 | 875 |
| Extended stem | 707 | 876 |
| Extended stem | 708 | 877 |
| Extended stem | 709 | 878 |
| Extended stem | 710 | 879 |
| Extended stem | 711 | 880 |
| Extended stem | 712 | 881 |
| Extended stem | 713 | 882 |
| Extended stem | 714 | 883 |
| Extended stem | 715 | 884 |
| Extended stem | 716 | 885 |
| Extended stem | 717 | 886 |
| Extended stem | 718 | 887 |
| Extended stem | 719 | 888 |
| Extended stem | 720 | 889 |
| Extended stem | 721 | 890 |
| Extended stem | 722 | 891 |
| Extended stem | 723 | 892 |
| Extended stem | 724 | 893 |
| Extended stem | 725 | 894 |
| Extended stem | 726 | 895 |
| Extended stem | 727 | 896 |
| Extended stem | 728 | 897 |
| Extended stem | 729 | 898 |
| Extended stem | 730 | 899 |
| Extended stem | 731 | 900 |
| Extended stem | 732 | 901 |
| Extended stem | 733 | 902 |
| Extended stem | 734 | 903 |
| Extended stem | 735 | 904 |
| Extended stem | 736 | 905 |
| Extended stem | 737 | 906 |
| Extended stem | 738 | 907 |

Next, all possible pairwise combinations of the individual mutations in different regions of Table 46 were introduced into ERS 316, so that each modified region could be assessed individually and in combination with all other modified regions. Specifically, to generate combinations of mutations that are expected to enhance editing efficiency when combined together, the mutations were assigned to specific regions of the ERS, as described above, and only mutations affecting different regions were combined together. Note that mutations in this library may each individually be composed of double mutations, and so a “combination of mutations” as described in this Example may involve combining a double mutant affecting the 5′ end and a domain replacement of the extended stem, each of which are composed of multiple deviations from the reference scaffold, for example. The individual and pairwise combinations of each of these mutations resulted in 10,829 unique ERS sequences, each of which represents a considerable deviation from the ERS 316 sequence (SEQ ID NO: 156). The DNA sequences of the ERSs with combinations of the mutations of Tables 44 and 45 are provided in SEQ ID NOS: 908-11,567 and 22,228-23571, and the corresponding RNA sequences are provided in SEQ ID NOS: 11,568-22,227 and 23,572-24, and 915.

### Molecular Biology of Library Construction:

The designed library of ERSs is synthesized, and then amplified by PCR with primers specific to the library. These primers amplify additional sequence at the 5′ and 3′ ends of the library to introduce sequence recognition sites for the restriction enzyme SapI. The PCR amplicon is introduced into a plasmid backbone containing flanking SapI sites for replacement of the flanked region by the library with standard Golden Gate cloning procedures. In a second step, a spacer sequence is further introduced into the library of plasmid backbones using standard Golden Gate cloning procedures. Next-generation sequencing (NGS) is performed to validate that the ERSs are evenly represented in the plasmid library.

### Lentivirus Production:

Lentiviral particles are generated by transfecting LentiX HEK293T cells, seeded 24 hours prior, at a confluency of 70-90%. Plasmids containing the pooled ERS library are introduced to a second-generation lentiviral system containing the packaging and VSV-G envelope plasmids with polyethylenimine, in serum-free media. For particle production, media is changed 12 hours post-transfection, and viruses are harvested at 36-48 hours post-transfection. Viral supernatant is filtered using 0.45 μm PES membrane filters and diluted in cell culture media when appropriate, prior to addition to target cells.

Screening and/or Selecting for Key Characteristics of ERS Function:

Screening and/or selections systems are developed to identify ERSs that are improved for a key functional property, such as folding stability of individual regions within the gRNA, folding stability of the entire gRNA, transcriptional efficiency, binding affinity to the CasX nuclease; and increased editing activity and editing specificity of the CasX RNP in complex with a target. Each of these functional changes is anticipated to result in higher editing of a DNA duplex; thus, screening systems are designed to identify ERSs out of a pool that are effective at a gene-editing based knockdown of reporter gene(s) in mammalian cells. CasX proteins 515, 593, 676, or 812 are used in the screening assays.

The screening methods may take several forms. For example, a gene encoding an endogenous cell surface receptor is edited so that its corresponding protein levels are knocked down, which would enable sorting away cells that maintain expression of the receptor. An antibody conjugated to a fluorophore or ligand enables distinguishing cells that maintain or lose receptor expression. Alternately, certain cell surface receptors that internalize toxins are targeted, such that application of a toxin is used to isolate only the cells that lose receptor expression. The representation of ERSs before and after selection is compared to generate a quantitative enrichment score for each ERS that reads out its efficacy in expressing in human cells, forming a complex with the RNP, and creating efficacious indels that reduce receptor expression. Screens and selections are repeated several times with different spacers, target genes, or other conditions to select for different functional outcomes of ERSs. Representative assays are described in International Publication No. WO2022120095A1.

In the case of cell screens, reporter cells are passed 24-48 hours prior to transduction to ensure cellular division occurs. At the point of transduction with lentiviral particles, the cells are trypsinized, counted, and diluted to appropriate density. Cells are resuspended with no treatment, library- or control-containing neat lentiviral supernatant at a low multiplicity of infection (MOI) to minimize dual lentiviral integrations. The lentiviral-cellular mixtures are seeded at 40-60% confluency prior to incubation at 37° C., 5% CO₂. Cells are selected for successful transduction 48 hours post-transduction with puromycin at 1-3 g/ml for 4-6 days followed by recovery in HEK or Fb medium.

Following selection, cells are suspended in 4′,6-diamidino-2-phenylindole (DAPI) and phosphate-buffered saline (PBS). Cells are then filtered by Corning™ strainer-cap FACS tube (Prod. 352235) and sorted using a Sony MA900 cell sorter. Cells are sorted for knockdown of a fluorescent reporter, in addition to gating for single, live cells via standard methods. Sorted cells from the experiment are lysed, and the genomic DNA is extracted using a Zymo Quick-DNA™ Miniprep Plus following the manufacturer's protocol.

### Sample Processing for NGS:

Genomic DNA is amplified via PCR with primers specific to the guide RNA-encoding DNA, to form a target amplicon. These primers contain additional sequence at the 5′ ends to introduce Illumina® read 1 and read 2 sequences. Standard PCR conditions are utilized to generate amplified DNA. Amplified DNA product is purified with Ampure XP DNA cleanup kit. Quality and quantification of the amplicon is assessed using a Fragment Analyzer DNA analyzer kit (Agilent, dsDNA 35-1500 bp). Amplicons is sequenced on the Illumina® Miseq™ (v3, 150 cycles of single-end sequencing) according to the manufacturer's instructions.

### NGS Analysis (Sample Processing and Data Analysis):

Reads are trimmed for adapter sequences with cutadapt (version 2.1), and the guide sequence (comprising the ERS sequence and spacer sequence) are extracted for each read (also using cutadapt v 2.1 linked adapters to extract the sequence between the upstream and downstream amplicon sequence). Unique guide RNA sequences are counted, and then each ERS sequence is compared to the list of designed ERS sequences and to the sequence of ERS 316 (SEQ ID NO: 156) to determine the identity of each. Read counts for each unique guide RNA sequence is normalized for sequencing depth using mean normalization. The change in representation before and after selection is quantified for each ERS by calculating an enrichment score (normalized read count in selected divided by normalized read count in naive). Two enrichment scores from different selections are combined by a weighted average of the individual log 2 enrichment scores, weighted by their relative representations within the naive population. Error on the log 2 enrichment scores are estimated calculating a 95% confidence interval on the average enrichment score across triplicate samples. These errors are propagated when combining the enrichment values for the two separate selections. Enrichment scores are analyzed for the effect of each region sequence either by itself, or in combination with other ERS sequences. Effective combinations are evaluated for the functional effects listed above.

### Results:

The screens and selections described herein are expected to identify ERSs with improved functional properties. Specifically, ERSs with improved binding to CasX, improved function in promoting gene editing (in the context of an RNP), improved function in promoting editing specificity (in the context of an RNP), and improved manufacturability are expected to be identified.

### Example 12: Generation and Assessment of Engineered Ribonucleic Acid Scaffolds with Mutations in the Pseudoknot Stem

As described in Example 9, ERS 320 was designed with mutations to deplete the CpG content of the DNA encoding the pseudoknot stem and the extended stem regions of the scaffold. In the experiment described in Example 9, ERS 320 produced a significant increase in editing potency relative to scaffold 235. This suggested that mutations to the pseudoknot stem have the potential to improve ERS function. In the following example, a selection of ERSs with mutations in the pseudoknot stem were designed and tested for their ability to promote genome editing.

### Materials and Methods

Design of ERSs with Mutations in the Pseudoknot Stem:

ERSs with mutations in the pseudoknot stem were designed based on ERS 316 (SEQ ID NO: 156). The positions of the mutations, as well as full-length DNA and RNA sequences of the ERSs are provided in Table 47, below. ERS 392 recapitulates the CG->GC mutation in the pseudoknot stem that was used to generate ERS 320, as described in Example 9. Scaffolds 174 and 235 and ERS 316 were included in this experiment as controls.

TABLE 47
Mutations and DNA and RNA sequences of guide
scaffolds and ERSs

| | | DNA | RNA |
|---|---|---|---|
| | | SEQ | SEQ |
| Scaffold/ | | ID | ID |
| ERS no. | Position of mutation* | NO | NO |
| 174 | n/a | 49700 | 17 |
| 235 | n/a | 49701 | 75 |
| 316 | n/a | 625 | 156 |
| 320 | 5.CG.GC;28.CG.GC;64.TCCCTC | 535 | 160 |
| | TTCGGAGGGA.CGCTTAGGGACTTCG | | |
| | GTCCCTAAGAG | | |
| 332 | 5.CG.GC;28.CG.GC;74.—.A | 547 | 172 |
| 376 | 6.—.T | 49702 | 49719 |
| 377 | 6.—.G | 49703 | 49720 |
| 378 | 6.—.G;26.A.T | 49704 | 49721 |
| 379 | 6.—.G;27.—.G | 49705 | 49722 |
| 380 | 6.—.T;28.—.C | 49706 | 49723 |
| 381 | 6.—.C;27.—.G | 49707 | 49724 |
| 382 | 28.—.C | 49708 | 49725 |
| 383 | 28. T | 49709 | 49726 |
| 384 | 6.—.G;28.—.C | 49710 | 49727 |
| 385 | 6.—.C | 49711 | 49728 |
| 386 | 5.C.T;6.—.G | 49712 | 49729 |
| 387 | 6. G | 49713 | 49730 |
| 388 | 6.—.G;28.—.C | 49714 | 49731 |
| 389 | 2.TGGC.CTGTAG;26.—.CTAC | 49715 | 49732 |
| 390 | 2.TGGC.CAGCAA;26.—.TTGC | 49716 | 49733 |
| 391 | 2.TGGC.CGAGAC;26.A.GGCTC | 49717 | 49734 |
| 392 | 5.CG.GC;28.CG.GC | 49718 | 49735 |

*positions are numbered relative to the 5′ end of ERS 316, with “0” being the 5′ terminus. Each mutation is indicated by its position, reference sequence, and alt sequence, separated by ‘.’ (e.g., 1.C.A is nucleotide position 1, reference sequence nucleotide C, alternate sequence nucleotide A). Position indexing starts at 0 such that the first base in ERS 316 is 0.A. Insertions are indicated with a ‘—’ in the reference sequence, and deletions with a ‘—’ in the alt sequence. Multiple individual mutations are semi-colon separated.

### Transfection and Assessment of B2M Editing:

HEK293T cells were lipofected with 100 ng of plasmid encoding CasX 515 and a gRNA made up of a scaffold or ERS listed in Table 47. The gRNAs had either a non-targeting spacer or a spacer targeting the B2M locus, as listed in Table 48. 24 hours post-transfection, cells were selected with 1 g/mL puromycin for 48 hours, and then allowed to recover for 24 hours. Then, cells were harvested for B2M protein expression analysis via immunostaining of the B2M-dependent HLA protein, followed by flow cytometry using the Attune™ NxT flow cytometer. Each construct was tested in duplicate, and the transfection and subsequent experiment was performed on two separate occasions.

TABLE 48
Sequences of B2M and non-targeting spacers
used in this example

| Spacer ID | DNA sequence | SEQ ID NO |
|---|---|---|
| 7.9 | GTGTAGTACAAGAGATAGAA | 49740 |
| 7.19 | CCCCCACTGAAAAAGATGAG | 49741 |
| 7.43 | AGGCCAGAAAGAGAGAGTAG | 49742 |
| 7.119 | CGCTGGATAGCCTCCAGGCC | 49743 |
| 7.14 | TGAAGCTGACAGCATTCGGG | 49744 |
| Non-targeting | CGAGACGTAATTACGTCTCG | 49745 |

### Lentiviral Transduction and Assessment of B2M Editing:

In a separate experiment, HEK293T cells were transduced with lentiviral particles encoding CasX 515 and a gRNA made up of either scaffold 174, scaffold 235, ERS 316, ERS 382, or ERS 392. The gRNAs had either a non-targeting spacer or spacer 7.9, 7.19, or 7.119 targeting the B2M locus, as provided in Table 48. Lentiviral particles were produced as described in Example 11, above. Viral supernatants were filtered using 0.45 μm membrane filters, diluted in media, and added to HEK293T target cells cultured at a relatively low multiplicity of infection (MOI) of either 0.1 or 0.05. Transduced cells were grown for three days in a 37° C. incubator with 5% C02. Cells were harvested for B2M protein expression analysis via immunostaining of the B2M-dependent HLA protein, followed by flow cytometry using the Attune™ NxT flow cytometer. The lentiviruses also expressed mScarlet, and the mean fluorescence intensity (MFI) of mScarlet was quantified to confirm that the cells contained similar amounts of transduced lentivirus.

### Results:

Editing of the B2M locus was measured in HEK293T cells transfected with plasmids expressing CasX 515 and gRNAs made up of the scaffolds or ERSs listed in Table 47. The results are provided in Table 49, below.

TABLE 49
Percentage of HEK293T cells with edited B2M locus following
transfection with plasmids expressing CasX 515 and
gRNAs with mutations to the pseudoknot stem

| | Percentage of HLA- HEK293T cells | |
|---|---|---|
| Scaffold | with edited B2M locus* | |
| ERS no. | First transfection | Second transfection |

Non-targeting spacer

| | | | | |
|---|---|---|---|---|
| 174 | 24.3 | 22.5 | 4.2 | 5.2 |
| 235 | 1.7 | 1.4 | 3.5 | 4.3 |
| 316 | — | 4.1 | 3.1 | 3.9 |
| 316 | 1 | 3.8 | 3.6 | 4.5 |
| 320 | 1.4 | 1.4 | 2.5 | 3.1 |
| 332 | 1.6 | 1.6 | 2.5 | 3.6 |
| 376 | 3.2 | 4.9 | 4.3 | 5.4 |
| 377 | 0.6 | 5.6 | 5 | 6.3 |
| 378 | 0.8 | 4.8 | 4.3 | 6 |
| 379 | 1.3 | 5.6 | 5 | 6.7 |
| 380 | 1.3 | 4.8 | 4.7 | 5.1 |
| 381 | 1.3 | 5.1 | 4.6 | 7.3 |
| 381 | 6.9 | 5.9 | 4.3 | 7 |
| 382 | 1.2 | 4.5 | 3.8 | 6.4 |
| 383 | 1.2 | 3.7 | 3.1 | 5.1 |
| 384 | 1.6 | 5.3 | 2.4 | 5.3 |
| 385 | 1.4 | 1.2 | 3.3 | 3.5 |
| 386 | 1.5 | 1.2 | 2.3 | 2.7 |
| 387 | 1.5 | 1.1 | 3 | 3.7 |
| 388 | 2.7 | 1.7 | 5.5 | 6.3 |
| 389 | 1.6 | 1.2 | 2.4 | 3.1 |
| 390 | 1.3 | 1.3 | 2.4 | 3.1 |
| 391 | 1.4 | 1.3 | 2.6 | 3.4 |
| 392 | 1.6 | 1.4 | 3 | 4.2 |

Spacer 7.9

| | | | | |
|---|---|---|---|---|
| 174 | 68.5 | 62.1 | 85.4 | 80.6 |
| 235 | 73.1 | 68.3 | 91.8 | 88.9 |
| 316 | — | 85.5 | 82.7 | 84.7 |
| 316 | 72.6 | 92.2 | 91.4 | 92.2 |
| 320 | 75.4 | 70.4 | 92.6 | 91.2 |
| 332 | 70.5 | 63.5 | 85.2 | 84.4 |
| 376 | 67.8 | 90.6 | 89.9 | 90 |
| 377 | 71.3 | 90.9 | 90.4 | 90.3 |
| 378 | 75.5 | 90.8 | 91.2 | 92.2 |
| 379 | 68 | 87.9 | 87.6 | 87.4 |
| 380 | 65.2 | 90.5 | 90.6 | 90.8 |
| 381 | 67 | 88.7 | 88.7 | 89 |
| 381 | 61.6 | 86.9 | 88 | 88 |
| 382 | 71.9 | 89.1 | 90.5 | 90.6 |
| 383 | 64.7 | 91.6 | 91.1 | 91 |
| 384 | 62.5 | 79.7 | 82 | 83.1 |
| 385 | 68.4 | 62.5 | 83.2 | 81.1 |
| 386 | 72.5 | 63.1 | 90.7 | 91.1 |
| 387 | 74.5 | 69.4 | 94 | 92.9 |
| 388 | 67.8 | 65.1 | 89.9 | 90.1 |
| 389 | 32.2 | 28.9 | 54.1 | 53.8 |
| 390 | 34.5 | 30.9 | 57.8 | 57.7 |
| 391 | 25.9 | 25.7 | 55.2 | 55.4 |
| 392 | 77.1 | 71.5 | 93.5 | 91.6 |

Spacer 7.19

| | | | | |
|---|---|---|---|---|
| 174 | 58.6 | 47.5 | 81.4 | 81.7 |
| 235 | 62.5 | 54.2 | 90.3 | 89.8 |
| 316 | — | 82.6 | 78.8 | 83.5 |
| 316 | 63.3 | 82.9 | 86.4 | 86.9 |
| 320 | 67.4 | 60.3 | 91.7 | 92.1 |
| 332 | 1.8 | 1.5 | 4.8 | 7.1 |
| 376 | 52.5 | 80 | 78.8 | 80.2 |
| 377 | 56.6 | 83.4 | 84 | 83.4 |
| 378 | 57.1 | 83.8 | 82.9 | 84.2 |
| 379 | 49.2 | 72.4 | 75.8 | 76 |
| 380 | 45.6 | 73.6 | 74.4 | 76.1 |
| 381 | 49.2 | 70.7 | 74.7 | 76.2 |
| 381 | 43.5 | 73.5 | 75.5 | 78.3 |
| 382 | 59.7 | 81.9 | 84.7 | 86.6 |
| 383 | 50.4 | 78.7 | 78.9 | 80.6 |
| 384 | 45.7 | 78.9 | 76.6 | 77.5 |
| 385 | 54.3 | 48.9 | 72.7 | 71.6 |
| 386 | 56.3 | 47.6 | 76.5 | 77.7 |
| 387 | 64.5 | 59.1 | 86.9 | 89.7 |
| 388 | 48 | 45.3 | 75.5 | 77.6 |
| 389 | 3.1 | 2.6 | 8.9 | 12.9 |
| 390 | 18 | 15.4 | 36.4 | 38.9 |
| 391 | 14.7 | 12.8 | 38.5 | 40.3 |
| 392 | 64.3 | 58.4 | 87.6 | 87.8 |

Spacer 7.43

| | | | | |
|---|---|---|---|---|
| 174 | 50.6 | 43.9 | 76.2 | 75.4 |
| 235 | 58.7 | 52.5 | 80.4 | 80.2 |
| 316 | — | 68 | 67.1 | 70.5 |
| 316 | 52.2 | 78.2 | 77.7 | 78.4 |
| 320 | 59.6 | 53.2 | 83.9 | 82.1 |
| 332 | 49.3 | 40.5 | 69.9 | 67.6 |
| 376 | 56.6 | 80.2 | 81.6 | 81.3 |
| 377 | 64.7 | 81.2 | 84.1 | 83.2 |
| 378 | 56.5 | 76.8 | 79.6 | 78.8 |
| 379 | 59.5 | 77.1 | 79.2 | 78.9 |
| 380 | 58.3 | 77.1 | 78.9 | 78.1 |
| 381 | 50.9 | 71.4 | 74.2 | 70.1 |
| 381 | 47.5 | 66.6 | 70.7 | 65.5 |
| 382 | 57.1 | 75.3 | 80.7 | 79.8 |
| 383 | 52.9 | 72.5 | 75.3 | 73.4 |
| 384 | 46.6 | 71.6 | 74.6 | 70.7 |
| 385 | 56.7 | 46.9 | 70.9 | 73.4 |
| 386 | 62.5 | 48.2 | 80.5 | 82.2 |
| 387 | 56.8 | 49.3 | 81.8 | 82.6 |
| 388 | 53.5 | 48.9 | 75.1 | 77.2 |
| 389 | 4.5 | 4 | 13.6 | 18 |
| 390 | 15.1 | 11.7 | 29.5 | 34.5 |
| 391 | 16.2 | 13.4 | 33.6 | 38.7 |
| 392 | 53.8 | 51.8 | 82 | 82.1 |

Spacer 7.119

| | | | | |
|---|---|---|---|---|
| 174 | 36.2 | 32.2 | 64.6 | 63.6 |
| 235 | 60.9 | 55.5 | 89 | 87.3 |
| 316 | — | 66.5 | 68.5 | 70.6 |
| 316 | 56.4 | 73.7 | 79.5 | 83.3 |
| 320 | 54.6 | 46.8 | 85.3 | 83.9 |
| 332 | 51.7 | 46.1 | 74.8 | 78.2 |
| 376 | 45.8 | 66.9 | 71.8 | 75.1 |
| 377 | 49 | 69.4 | 76.3 | 79.2 |
| 378 | 45.7 | 67 | 75.1 | 78.1 |
| 379 | 35.2 | 49.9 | 59.3 | 61.8 |
| 380 | 37.9 | 56.2 | 66.2 | 67.3 |
| 381 | 31.9 | 50.2 | 52.4 | 56.8 |
| 381 | 28.9 | 46.2 | 50.2 | 52.5 |
| 382 | 49.8 | 67.8 | 71.3 | 76.3 |
| 383 | 44.2 | 60.9 | 65.5 | 69.6 |
| 384 | 36.8 | 59.3 | 64.9 | 66 |
| 385 | 39.1 | 32.8 | 62.8 | 62.7 |
| 386 | 40.9 | 33.8 | 72.7 | 74.5 |
| 387 | 7.8 | 6.2 | 24.1 | 31.4 |
| 388 | 35.8 | 30.6 | 62.2 | 64.4 |
| 389 | 4.9 | 3.9 | 15.3 | 20 |
| 390 | 4.3 | 3.9 | 11.9 | 17.1 |
| 391 | 3.8 | 3 | 9.3 | 12.9 |
| 392 | 58 | 50.2 | 85 | 83.3 |

Spacer 7.14

| | | | | |
|---|---|---|---|---|
| 174 | 47.5 | 43.7 | 74.1 | 76.4 |
| 235 | 31.4 | 29.6 | 58 | 57.2 |
| 316 | — | 44.8 | 41.6 | 40.3 |
| 316 | 31.8 | 52.7 | 53.2 | 55.6 |
| 320 | 36.4 | 34 | 59.9 | 62.3 |
| 332 | 14.8 | 12.4 | 25 | 25.4 |
| 376 | 34.1 | 55.5 | 56.1 | 56.9 |
| 377 | 24.6 | 45.1 | 42.7 | — |
| 378 | 26.9 | 45.1 | 43.5 | 47.5 |
| 379 | 36.1 | 54.8 | 56.2 | 59.3 |
| 380 | 46.6 | 67.4 | 70.2 | 72.3 |
| 381 | 50.8 | 72.1 | 73.4 | 74 |
| 381 | — | 39.3 | 35.2 | 39.4 |
| 382 | 42.2 | 65.6 | 66.4 | 67 |
| 383 | 37 | 64 | 64.9 | 64.9 |
| 384 | 18 | 38.4 | 35.1 | 38.1 |
| 385 | 18.1 | 16.1 | 29.6 | 24 |
| 386 | 36.2 | 31.6 | 53.1 | 53.8 |
| 387 | 33.3 | 28.2 | 48.1 | 51 |
| 388 | 31 | 25.5 | 49.3 | 55.4 |
| 389 | 20.6 | 17.6 | 45 | 45.8 |
| 390 | 8.4 | 7.9 | 19.3 | 22.6 |
| 391 | 52.8 | 52.9 | 80.9 | 78.9 |
| 392 | 42.3 | 42.6 | 66.7 | 67.2 |

*Data are shown rounded to the nearest tenth.

Many of the tested ERS produced levels of editing that were similar to ERS 316 (Table 49). Surprisingly, some scaffolds produced higher levels of editing than ERS 316, but only with certain spacers. Specifically, scaffold 391 showed a relatively high level of editing with spacer 7.14, but not other spacers. Scaffold 392 produced overall high levels of editing with multiple spacers, and edited with spacer 7.14 to a greater extent than ERS 316. The sequences of ERS 391 and 392 are outlined compared to scaffold 23 and ERS 316 in Table 50, below.

TABLE 50
Sequences of regions of scaffold 235,
ERS 316, and ERS 392, 5′ to 3′

| | | |
|---|---|---|
| | | SEQ |
| | | ID |
| Region of Scaffold | RNA Sequence* | NO |

Guide scaffold 235

| | | |
|---|---|---|
| 5′ end | AC | — |
| Pseudoknot stem I | UGGCGCU | — |
| Triplex loop (including | UCUAUCUGAUUACUCUG | 49736 |
| triplex regions I and II) | | |
| Pseudoknot stem II | AGCGCCA | — |
| Linking nucleotides I | UCA | |
| Scaffold stem loop | CCAGCGACUAUGUCGUA | 49737 |
| | GUGG | |
| Linking nucleotides II | GUAAA | — |
| Extended stem loop | GCCGCUUACGGACUUCG | 49738 |
| | GUCCGUAAGAGGC | |
| Linking nucleotides III | AU | — |
| Triplex region III | CAGAG | — |

ERS 316

| | | |
|---|---|---|
| 5′ end | AC | — |
| Pseudoknot stem I | UGGCGCU | — |
| Triplex loop (including | UCUAUCUGAUUACUCUG | 49736 |
| triplex regions I and II) | | |
| Pseudoknot stem II | AGCGCCA | — |
| Linking nucleotides I | UCA | — |
| Scaffold stem loop | CCAGCGACUAUGUCGUA | 49737 |
| | GUGG | |
| Linking nucleotides II | GUAAA | — |
| Extended stem loop | GCUCCCUCUUCGGAGGG | 49739 |
| | AGC | |
| Linking nucleotides III | AU | — |
| Triplex region III | CAGAG | — |

ERS 391

| | | |
|---|---|---|
| 5′ end | AC | — |
| Pseudoknot stem I | CGAGACGCU | — |
| Triplex loop (including | UCUAUCUGAUUACUCUG | 49736 |
| triplex regions I and II) | | |
| Pseudoknot stem II | GGCUCGCGCCA | — |
| Linking nucleotides I | UCA | — |
| Scaffold stem loop | CCAGCGACUAUGUCGUA | 49737 |
| | GUGG | |
| Linking nucleotides II | GUAAA | — |
| Extended stem loop | GCUCCCUCUUCGGAGGG | 49739 |
| | AGC | |
| Linking nucleotides III | AU | — |
| Triplex region III | CAGAG | — |

ERS 392

| | | |
|---|---|---|
| 5′ end | AC | — |
| Pseudoknot stem I | UGGGCCU | — |
| Triplex loop (including | UCUAUCUGAUUACUCUG | 49736 |
| triplex regions I and II) | | |
| Pseudoknot stem II | AGGCCCA | — |
| Linking nucleotides I | UCA | — |
| Scaffold stem loop | CCAGCGACUAUGUCGUA | 49737 |
| | GUGG | |
| Linking nucleotides II | GUAAA | — |
| Extended stem loop | GCCGCUUACGGACUUCG | 49738 |
| | GUCCGUAAGAGGC | |
| Linking nucleotides III | AU | — |
| Triplex region III | CAGAG | |

*Bases that form the triplex (herein termed triplex regions I-III) are bolded and underlined.

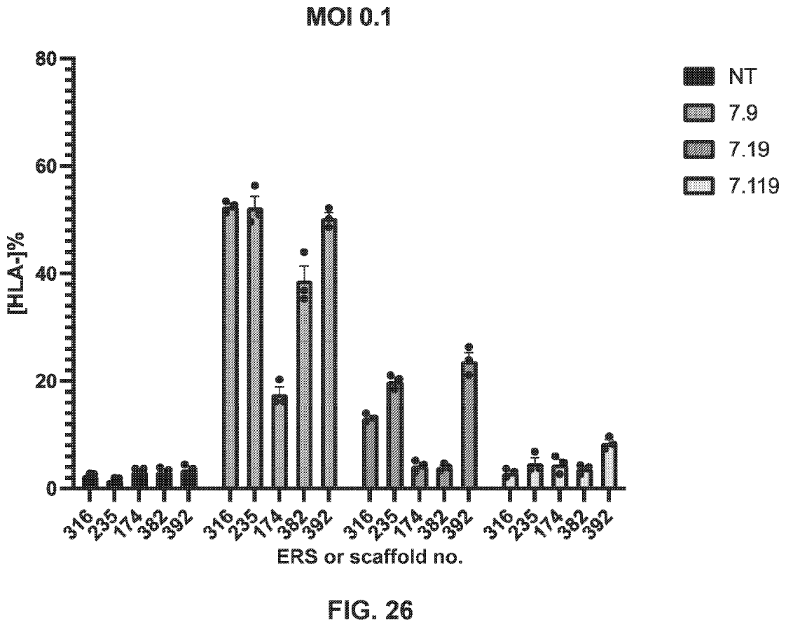

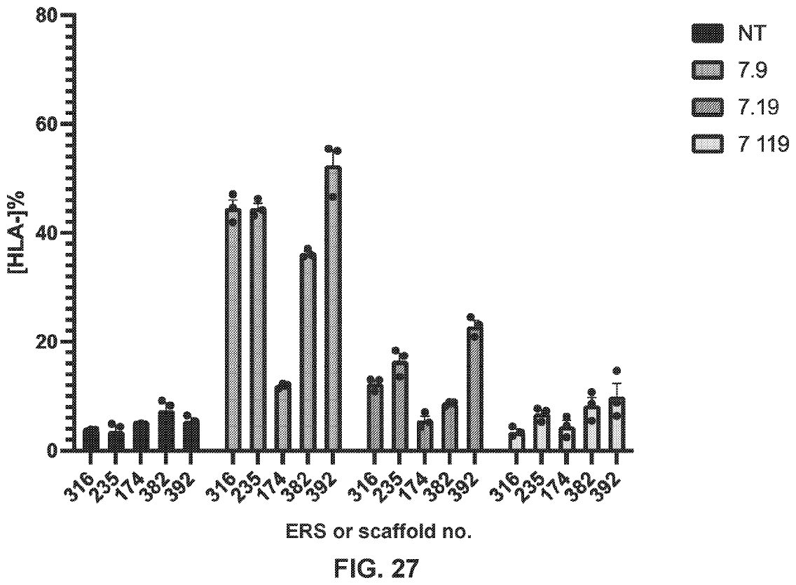

Scaffold 174, scaffold 235, ERS 316, ERS 382, or ERS 392 were also tested via lentiviral transduction in HEK293T cells at MOIs of 0.1 (FIG. 26) and 0.05 (FIG. 27). At these relatively low MOIs, the improvement in editing activity in scaffold 235 and ERS 316 relative to scaffold 174 was pronounced, with both scaffold 235 and ERS 316 producing over twice as many cells with edited B2M loci as scaffold 174. These results show that scaffold 235 and ERS 316 are highly effective scaffolds for producing gene editing at low doses in cell culture, and are therefore also expected to be highly useful scaffolds for editing in vivo. In these assays, ERS 392 produced similar levels of editing to ERS 316 with the tested spacers.

Overall, the results described herein demonstrate that ERSs with mutations in the pseudoknot stem region can produce gene editing.

### Example 13: Assessment of CpG-Depleted CasX 515 Variants on CasX-Mediated Editing

Experiments were performed to deplete CpG motifs in the AAV construct encoding for CasX protein 515 and demonstrate that these CpG-depleted CasX 515 variants can edit effectively in vitro.

### Materials and Methods

### Design of CpG-Depleted and Codon-Optimized CasX 515 Variants and AAV Plasmid Cloning:

Nucleotide substitutions to replace native CpG motifs in CasX protein 515, as well as the flanking c-MYC NLSes, were rationally designed with codon optimization using various publicly available algorithms. As a result, the amino acid sequence of the encoding sequence of CpG-depleted CasX 515 with flanking c-MYC NLSes would be the same as the amino acid sequence of the corresponding encoding sequence of native CasX 515 with flanking c-MYC NLSes. Table 51 provides the sequences of CpG-depleted and codon-optimized variants of CasX 515 with flanking c-MYC NLSes, as well as the corresponding non-CpG depleted CasX 515 with flanking c-MYC NLSes.

TABLE 51
Sequences of CpG-depleted and codon-optimized
variants of CasX 515 with flanking c-MYC NLSes

| CpG-depleted or non-CpG-depleted | | |
|---|---|---|
| variant 515 with flanking | | |
| c-MYC NLSes (version no.) | AAV construct ID | SEQ ID NO |
| CpG-depleted 515 (v1) | 290 | 49850 |
| CpG-depleted 515 (v2) | 291 | 49851 |
| CpG-depleted 515 (v3) | 292 | 49852 |
| CpG-depleted 515 (v4) | 293 | 49853 |
| CpG-depleted 515 (v5) | 294 | 49854 |
| CpG-depleted 515 (v6) | 295 | 49855 |
| CpG-depleted 515 (v7) | 296 | 49856 |
| CpG-depleted 515 (v8) | — | 49857 |
| CpG-depleted 515 (v9) | — | 49858 |
| CpG-depleted 515 (v10) | — | 49859 |
| CpG-depleted 515 (v11) | 297 | 49860 |
| CpG-depleted 515 (v12) | — | 49861 |
| Non-CpG-depleted 515 | 298 | 49862 |

All resulting sequences of the CpG-depleted and codon-optimized variants of CasX 515 with flanking c-MYC NLSes were cloned into a base AAV plasmid (sequences shown in Table 52). gRNA scaffold 235 and spacer 31.63, which targets the AAVS1 locus, were used for the experiments discussed in this example. The resulting AAV constructs were generated using standard molecular cloning techniques. Cloned and sequence-validated plasmid constructs were midi-prepped for subsequent nucleofection and AAV vector production.

TABLE 52
Sequences encoding for a base AAV plasmid into which CpG-
depleted variants of CasX 515 in Table 51 were cloned

| AAV | | |
|---|---|---|
| construct | | DNA Sequence or |
| ID | Component Name | SEQ ID NO |
| 290 | 5′ ITR | 487 |
| through | buffer sequence | 49863 |
| 298 | UlA promoter | 49864 |
| | buffer sequence | 49865 |
| | Kozak | GCCACC |
| | start codon + CpG-depleted (or non- | See sequences |
| | CpG-depleted) & codon-optimized CasX | listed in Table 51 |
| | 515 with flanking c-MYC NLSes | |
| | stop codon | TGA |
| | buffer sequence | 49870 |
| | bGH poly(A) signal sequence | 49866 |
| | buffer sequence | GGTACCGT |
| | U6 promoter | 49867 |
| | buffer sequence | GAAACACC |
| | Scaffold 235 | 49701 |
| | AAVS1 spacer (31.63) | 49868 |
| | buffer sequence | 49869 |
| | 3′ ITR | 488 |

### Transfection of HEK293 Cells In Vitro:

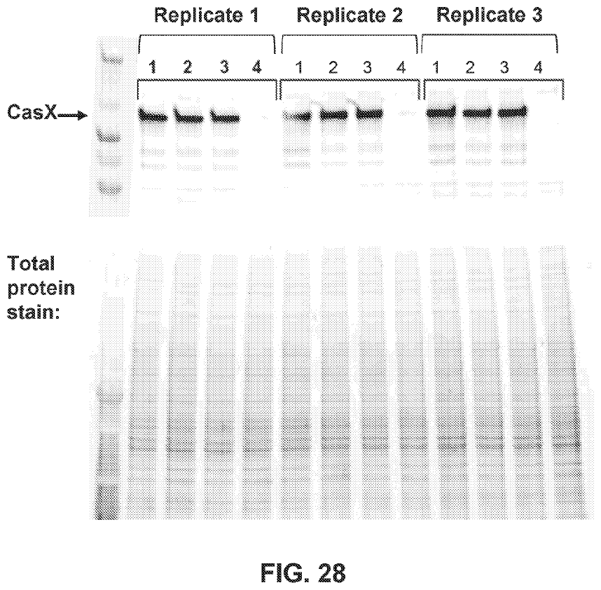

˜50,000 HEK293 cells per well were seeded on 24-well plates; two days later, cells were transfected with AAV plasmids containing sequences for a non-CpG-depleted (CpG⁺) CasX 515 (Table 51) or a version 1 of a CpG-depleted and codon-optimized CasX 515 variant (CpG⁻ v1; Table 51) following standard methods using lipofectamine. Two days later, cells were harvested to extract total protein lysate for western blotting analysis. Quantification of protein concentration and western blotting were performed using standard procedures. Three technical replicates were performed (Replicates 1-3) for the western blot. The results of this experiment are shown in FIG. 28. Untransfected cells served as an experimental control.

### AAV Production and Titering:

AAV production was performed using methods described in Example 9. AAV titering was performed by ddPCR using a primer-probe set specific to bGH, an indicator of the AAV transgene.

### AAV Transduction of iNs (Induced Neurons) In Vitro:

For one experiment, ˜30,000 iNs per well were seeded Matrigel-coated 96-well plates 7 days prior to transduction. Cells were transduced with AAVs expressing the CasX gRNA system, a non-CpG-depleted CasX 515 (CpG⁺; Table 51) or version 1 of the CpG-depleted and codon-optimized CasX 515 variant (CpG⁻ v1; Table 51) and codon-optimized variants of CasX 515, at an MOI of 1E4 vg/cell. 7 days post-transduction, cells were harvested for gDNA extraction for editing analysis at the AAVS1 locus using NGS. One replicate was performed this experiment, and the results are shown in Table 53.

### AAV Transduction of HEK293 Cells In Vitro:

In a second experiment, ˜5,000 HEK293 cells per well are seeded on 96-well plates two days prior to transduction. AAVs expressing the CasX:gRNA system, containing various CpG-depleted and codon-optimized variants of CasX 515, are diluted in neuronal plating media and added to cells. Cells are transduced at four MOIs (1E4, 3E3, 1E3, or 3.7E2 vg/cell). Five days post-transduction, cells are harvested for gDNA extraction for editing analysis at the AAVS1 locus using NGS.

### Results:

In one experiment, HEK293 cells were transiently transfected with AAV plasmids containing a CpG⁺ CasX 515 sequence or CpG− v1 CasX 515 sequence. Four days post-transfection, CasX expression and editing activity at the AAVS1 locus were evaluated by western blotting and NGS respectively. The results of the western blotting analysis are portrayed in FIG. 28, showing CasX protein levels in transfected HEK293 cells, with a total protein stain blot (bottom blot) serving as the loading control. Cells transfected with the AAV plasmid containing a CpG⁺ CasX 515 sequence are labeled as “CpG⁺ CasX 515” (lane 1), while cells transfected with the construct harboring a CpG⁻ CasX 515 sequence are labelled as “CpG⁻ CasX 515_A” (lane 2) and “CpG⁻ CasX 515_B” (lane 3). Untransfected HEK293 cells are labelled “No plasmid control” (lane 4). The results in FIG. 28 show that expressing the AAV plasmid containing either the CpG⁻ or CpG⁺ CasX 515 sequence resulted in CasX expression. Editing activity at the AAVS1 locus was also assessed in human iNs; the results show that use of the AAV plasmid with either CpG⁻ v1 or CpG⁺ CasX 515 sequence resulting in editing at the target locus (Table 53).

TABLE 53
Results of the editing assay at the AAVS1 locus when using AAV
plasmid containing either CpG⁻ or CpG⁺ CasX 515

| Experimental condition | Indel rate at AAVS1 locus |
|---|---|
| AAV plasmid with CpG⁺ 515 | 20.82% |
| AAV plasmid with CpG⁻ 515 (v1) | 16.55% |
| ‘No plasmid’ control | 0.06% |

The experiments demonstrate that depleting CpG motifs in the AAV construct encoding for CasX protein 515 resulted in sufficient CasX expression to induce effective editing at the target locus in vitro. Incorporating CpG-depleted AAV elements into the AAV genome would potentially reduce the risk of immunogenicity post-delivery of AAVs into target cells and tissues.

What is claimed is:

1. An engineered ribonucleic acid scaffold (ERS) comprising a sequence of SEQ ID NO: 17, or a sequence having at least about 70% sequence identity thereto, comprising an extended stem loop sequence of SEQ ID NO: 49739 and one or more mutations at positions selected from the group consisting of U11, U24, A29, and A87.

2. The engineered ERS of claim 1, comprising mutations at positions U11, U24, A29, and A87.

3. The engineered ERS of claim 1, comprising one or more mutations selected from the group consisting of U11C, U24C, A29C, and A87G.

4. The engineered ERS of claim 3, comprising mutations consisting of U11C, U24C, A29C, and A87G.

5. An engineered ribonucleic acid scaffold (ERS) comprising a sequence of SEQ ID NO: 75, or a sequence having at least about 70% sequence identity thereto, modified to comprise an extended stem loop sequence of SEQ ID NO: 49739.

6. The ERS of claim 5, the sequence comprising regions selected from the group consisting of:
a. a 5′ end comprising a sequence of AC;
b. a pseudoknot stem I comprising a sequence of UGGCGCU;
c. a triplex loop comprising a sequence of SEQ ID NO: 49736;
d. a pseudoknot stem II comprising a sequence of AGCGCCA; and
e. a triplex region III comprising a sequence of CAGAG.

7. An engineered ribonucleic acid scaffold (ERS), comprising the sequence of ACUGGCGCUUCUAUCUGAUUACUCUGAGCGCCAUCACCAGCGACUAUGUCGUA GUGGGUAAAGCUCCCUCUUCGGAGGGAGCAUCAGAG (SEQ ID NO: 156), or a sequence having at least about 96% sequence identity thereto.

8. An engineered ribonucleic acid scaffold (ERS) comprising a sequence having at least about 70% sequence identity to (i) ACUGGCACUUCUAUCUGAUUACUCUGAGAGCCAUCACCAGCGACUAUGUCGUA UGGGUAAAGCCGCUUACGGACUUCGGUCCGUAAGAGGCAUCAGAG (SEQ ID NO: 61); or (ii) ACUGGCGCUUCUAUCUGAUUACUCUGAGCGCCAUCACCAGCGACUAUGUCGUA GUGGGUAAAGCUCCCUCUUCGGAGGGAGCAUCAGAG (SEQ ID NO: 156); comprising one or more modifications in the sequence, wherein the one or more modifications result in an improved characteristic compared to unmodified SEQ ID NO: 61 or SEQ ID NO: 156.

9. The ERS of claim 8, comprising at least two modifications in the sequence, wherein the modifications result in an improved characteristic compared to unmodified SEQ ID NO: 61 or SEQ ID NO: 156.

10. The ERS of claim 8 or claim 9, wherein the modification comprises:
a. a substitution of 1 to 30 consecutive nucleotides in one or more regions of the scaffold;
b. a deletion of 1 to 10 consecutive nucleotides in one or more regions of the scaffold;
c. an insertion of 1 to 10 consecutive nucleotides in one or more regions of the scaffold;
d. a substitution of the scaffold stem loop with an RNA stem loop sequence from a heterologous RNA source;
e. a substitution of the extended scaffold stem loop with an RNA stem loop sequence from a heterologous RNA source; or
f. any combination of (a)-(d).

11. The ERS of any one of claims 8-10, wherein the modifications comprise mutations in one or more regions selected from the group consisting of a 5′ end, a pseudoknot stem, a triplex loop, a scaffold stem loop, an extended stem loop, and a triplex region III.

12. The ERS of any one of claims 8-10, wherein the modifications comprise mutations in at least two regions of the ERS, wherein the regions are selected from the group consisting of a 5′ end, a pseudoknot stem I, a triplex loop, a pseudoknot stem II, a scaffold stem loop, an extended stem loop, and a triplex region III.

13. The ERS of any one of claims 8-12, wherein the mutations are selected from the group consisting of the mutations of Tables 44, 45, and 47.

14. The ERS of claim 13, wherein sequences of the individual mutated regions have the sequences of:
a. SEQ ID NOS: 739-753 in the 5′ end region;
b. SEQ ID NOS: 754-772 in the triplex loop region;
c. SEQ ID NOS: 773-791 in the triplex region;
d. SEQ ID NOS: 792-841 in the pseudoknot region;
e. SEQ ID NOS: 842-869 in the scaffold stem region; and/or
f. SEQ ID NOS: 870-907 in the extended stem region.

15. The ERS of claim 13, wherein the ERS comprises paired combinations of individual mutated sequences from different or the same regions.

16. The ERS of claim 15, wherein the ERS comprises a sequence selected from the group consisting of SEQ ID NOS: 11,568-22,227 and 23,572-24,915, or a sequence having at least 70% sequence identity thereto.

17. The ERS of claim 15, wherein the ERS comprises a sequence selected from the group consisting of SEQ ID NOS: 11,568-22,227 and 23,572-24,915.

18. The ERS of any one of claims 7-17, wherein the scaffold has 85-100 nucleotides, or any integer in between.

19. An ERS comprising a sequence selected from the group consisting of SEQ ID NOS: 156, 739-907, 739-907, 11568-22227, 23572-24915, and 49719-49735, or a sequence having at least about 90%, at least about 95%, at least about 98%, or at least about 99% sequence identity thereto, wherein the ERS comprises an improved characteristic compared to the sequence of SEQ ID NO: 17.

20. The ERS of claim 19, wherein the ERS comprises a sequence selected from the group consisting of SEQ ID NOS: 156, 739-907, 11568-22227, 23572-24915, and 49719-49735, wherein the ERS comprises an improved characteristic compared to the sequence of SEQ ID NO: 17, when assayed in an in vitro cell-based assay under comparable conditions.

21. The ERS of claim 19 or claim 20, wherein the improved characteristic is one or more functional properties selected from the group consisting of improved binding to a CasX nuclease to form a ribonucleoprotein (RNP), improved folding stability of the ERS, increased half-life in a cell, increased transcriptional efficiency, enhanced ability to synthetically manufacture the ERS, improved editing activity of a target nucleic acid by an RNP comprising the ERS, and improved editing specificity by an RNP comprising the ERS.

22. The ERS of any one of claims 1-21, wherein the ERS comprises one or more heterologous RNA sequences in the extended stem loop.

23. The ERS of claim 22, wherein the heterologous RNA is selected from the group consisting of a MS2 hairpin, Qβ hairpin, U1 hairpin II, Uvsx hairpin, and a PP7 stem loop, or sequence variants thereof.

24. The ERS of claim 22 or claim 23, wherein the heterologous RNA is capable of binding a protein, a RNA, a DNA, or a small molecule.

25. The ERS of any one of claims 1-24, wherein the ERS comprises a Rev response element (RRE), or a portion thereof.

26. The ERS of any one of claims 1-25, comprising a targeting sequence linked at the 3′ end of the ERS that is complementary to a target nucleic acid sequence.

27. The ERS of claim 26, wherein the targeting sequence has 15-20 nucleotides.

28. The ERS of claim 27, wherein the targeting sequence has 20 nucleotides.

29. The ERS of any one of claims 26-28, wherein the ERS and linked targeting sequence has 100-115 nucleotides.

30. The ERS of any one of claims 1-29, wherein the CpG content of the ERS is reduced or depleted.

31. The ERS of claim 30, wherein the CpG content is less than about 10%, less than about 5%, or less than about 1%.

32. The ERS of any one of claims 1-31, wherein the ERS comprises one or more chemical modifications to the sequence.

33. The ERS of claim 32, wherein the chemical modification is addition of a 2′O-methyl group to one or more nucleotides of the sequence.

34. The ERS of claim 32 or claim 33, wherein one or more nucleotides on either or both of the 5′ and 3′ terminal ends of the ERS are modified by an addition of a 2′O-methyl group.

35. The ERS of any one of claims 32-34, wherein the chemical modification is substitution of a phosphorothioate bond between two or more nucleosides of the sequence.

36. The ERS of any one of claims 32-35, wherein the chemical modification is a substitution of phosphorothioate bonds between two or more nucleotides on either or both of the 5′ and 3′ terminal ends of the ERS.

37. The ERS of any one of claims 32-36, wherein the chemically modified ERS comprises a sequence selected from the group consisting of SEQ ID NOS: 49750-49758, 49760-49768, and 49770-49749.

38. The ERS of any one of claims 32-37, wherein the chemically modified ERS comprises a sequence of SEQ ID NO: 49770.

39. The ERS of claim 37 or claim 38, wherein the chemically modified ERS sequence is modified with a 20 nucleotide targeting sequence complementary to a target nucleic acid.

40. The ERS of any one of claims 32-39, wherein the chemical modifications result in reduced susceptibility of the ERS to degradation by cellular RNase compared to an unmodified ERS.

41. The ERS of any one of claims 1-40, wherein the ERS is capable of forming a ribonucleoprotein (RNP) complex with a CasX protein.

42. An engineered CasX protein, comprising a sequence having at least two mutations in the sequence of CasX 515 (SEQ ID NO: 49699) wherein the mutations result in an improved characteristic compared to unmodified CasX 515.

43. The engineered CasX protein of claim, wherein the improved condition is determined in an in vitro assay under comparable conditions.

44. The engineered CasX protein of claim 42, wherein the mutations are selected from the group consisting of:
a. an amino acid substitution;
b. an amino acid deletion;
c. an amino acid insertion; and
d. any combination of (a)-(c).

45. The engineered CasX protein of any one of claim 42, wherein engineered CasX protein comprises:
a. an oligonucleotide binding domain (OBD)-I comprising an amino acid sequence comprising one or more mutations relative to the sequence of SEQ ID NO: 295;
b. a helical I-I domain comprising an amino acid sequence comprising one or more mutations relative to the sequence of SEQ ID NO: 296;
c. an NTSB domain comprising an amino acid sequence comprising one or more mutations relative to the sequence of SEQ ID NO: 297;
d. a helical I-II domain comprising an amino acid sequence comprising one or more mutations relative to the sequence of SEQ ID NO: 298;
e. a helical II domain comprising an amino acid sequence comprising one or more mutations relative to the sequence of SEQ ID NO: 299;
f. a RuvC-I domain comprising an amino acid sequence comprising one or more mutations relative to the sequence of SEQ ID NO: 301;
g. a target strand loading (TSL) domain comprising an amino acid sequence comprising one or more mutations relative to the sequence of SEQ ID NO: 302; or
h. any combination of (a)-(g).

46. The engineered CasX protein of claim 45, wherein:
a. the OBD-I comprises one or more mutations relative to the sequence of SEQ ID NO: 295 selected from the group consisting of an I3G substitution, an insertion of a G at position 4, a K4G substitution, an insertion of a G at position 5, a K8G substitution, an insertion of an R at position 26, and a R34P substitution;
b. the helical I-I domain comprises an R7Q substitution relative to the amino acid sequence of SEQ ID NO: 296;
c. the NTSB domain comprises one or more mutations relative to the sequence of SEQ ID NO: 297 selected from the group consisting of an L68K substitution, an L68Q substitution, an A70Y substitution, an A70D substitution, and an A70S substitution;
d. the helical I-II domain comprises one or more mutations relative to the sequence of SEQ ID NO: 298 selected from the group consisting of a G32T substitution, an M112T substitution, and an M112W substitution;
e. the helical II domain comprises one or more mutations relative to the sequence of SEQ ID NO: 299 selected from the group consisting of a Y65T substitution and an E148D substitution;
f. the RuvC-I domain comprises an S51R substitution relative to the sequence of SEQ ID NO: 301;
g. the TSL domain comprises one or more mutations relative to the sequence of SEQ ID NO: 302 selected from the group consisting of a V15M substitution, a T76D substitution, and an S80Q substitution; or
h. any combination of (a)-(g).

47. The engineered CasX protein of claim 45 or claim 46, wherein:
a. the OBD-I comprises a sequence selected from the group consisting of SEQ ID NOS: 295, 49800, 49803-49808, and 49822-49833, or a sequence having at least about 90%, at least about 95%, at least about 98%, at least about 99% sequence identity thereto;
b. the helical I-I domain comprises a sequence selected from the group consisting of SEQ ID NOS: 296 and 49809, or a sequence having at least about 90%, at least about 95%, at least about 98%, or at least about 99% sequence identity thereto;
c. the NTSB domain comprises a sequence selected from the group consisting of SEQ ID NOS: 297, 49802, 49810, 49811, 49812, 49818, and 49835-49840, or a sequence having at least about 90%, at least about 95%, at least about 98%, or at least about 99% sequence identity thereto;
d. the helical I-II domain comprises a sequence selected from the group consisting of SEQ ID NOS: 298, 49801, 49813-49814, and 49842, or a sequence having at least about 90%, at least about 95%, at least about 98%, or at least about 99% sequence identity thereto;
e. the helical II domain comprises a sequence selected from the group consisting of SEQ ID NOS: 299, 49815-49816, and 49843, or a sequence having at least about 90%, at least about 95%, at least about 98%, or at least about 99% sequence identity thereto;
f. the RuvC-I domain comprises a sequence selected from the group consisting of SEQ ID NOS: 301 and 49821, or a sequence having at least about 90%, at least about 95%, at least about 98%, or at least about 99% sequence identity thereto;
g. the TSL domain comprises a sequence selected from the group consisting of SEQ ID NOS: 302, 49817, 49819, 49820, and 49844-49846, or a sequence having at least about 90%, at least about 95%, at least about 98%, or at least about 99% sequence identity thereto; or
h. any combination of (a)-(g).

48. The engineered CasX protein of any one of claims 45-47, wherein the engineered CasX protein further comprises:
a. an OBD-II comprising the sequence of SEQ ID NO: 300, or a sequence having at least about 90%, at least about 95%, at least about 98%, or at least about 99% sequence identity thereto; and/or
b. a RuvC-II domain comprising the sequence of SEQ ID NO: 303, or a sequence having at least about 90%, at least about 95%, at least about 98%, or at least about 99% sequence identity thereto.

49. The engineered CasX protein of any one of claims 42-48, wherein the engineered CasX protein comprises, from N- to C-terminus, an OBD-I domain, a helical I-I domain, an NTSB domain, a helical I-II domain, a helical II domain, an OBD-II, a RuvC-I domain, a TSL domain, and a RuvC-II domain, with each domain comprising a sequence as set forth in Table 21.

50. The engineered CasX protein of any one of claims 42-49, wherein the two mutations are selected from the group consisting of the paired mutations as set forth in Table 22.

51. The engineered CasX protein of any one of claims 42-49, wherein the two mutations are selected from the group consisting of the following pairs: 4.I.G & 64.R.Q, 4I.G & 169.L.K, 4.I.G & 169.L.Q, 4.I.G & 171.A.D, 4.I.G & 171.A.Y, 4.I.G & 171.A.S, 4.I.G & 224.G.T, 4.I.G & 304.M.T, 4.I.G & 398.Y.T, 4.I.G & 826.V.M, 4.I.G & 887.T.D, 4.I.G & 891.S.Q, 5.-.G & 64.R.Q, 5.-.G & 169.L.K, 5.-.G & 169.L.Q, 5.-.G & 171.A.D, 5.-.G & 171.A.Y, 5.-.G & 171.A.S, 5.-.G & 224.G.T, 5.-.G & 304.M.T, 5.-.G & 398.Y.T, 5.-.G & 826.V.M, 5.-.G & 887.T.D, 5.-.G & 891.S.Q, 9.K.G & 64.R.Q, 9.K.G & 169.L.K, 9.K.G & 169.L.Q, 9.K.G & 171.A.D, 9.K.G & 171.A.Y, 9.K.G & 171.A.S, 9.K.G & 224.G.T, 9.K.G & 304.M.T, 9.K.G & 398.Y.T, 9.K.G & 826.V.M, 9.K.G & 887.T.D, 9.K.G & 891.S.Q, 27.-.R & 64.R.Q, 27.-.R & 169.L.K, 27.-.R & 169.L.Q, 27.-.R & 171.A.D, 27.-.R & 171.A.Y, 27.-.R & 171.A.S, 27.-.R & 224.G.T, 27.-.R & 304.M.T, 27.-.R & 398.Y.T, 27.-.R & 826.V.M, 27.-.R & 887.T D, 27.-.R & 891.S.Q, 35.R.P & 64.R.Q, 35.R.P & 169.L.K, 35.R.P & 169.L.Q, 35.R.P & 171.A.D, 35.R.P & 171.A.Y, 35.R.P & 171.A.S, 35.R.P & 224.G.T, 35.R.P & 304.M.T, 35.R.P & 398.Y. T, 35.R.P & 826.V.M, 35.R.P & 887.T.D, 35.R.P & 891.S.Q, 887.T.D & 891.S.Q, 64.R.Q & 169.L.K, 64.R.Q & 169.L.Q, 64.R.Q & 171.A.D, 64.R.Q & 171.A.Y, 64.R.Q & 171.A.S, 64.R.Q & 224.G. T, 64.R.Q & 304.M.T, 64.R.Q & 398.Y.T, 64.R.Q & 826.V.M, 64.R.Q & 887.T.D, 64.R.Q & 891.S.Q, 169.L.K & 171.A.D, 169.L.K & 171.A.Y, 169.L.K & 171.A.S, 169.L.K & 224.G.T, 169.L.K & 304.M.T, 169.L.K & 398.Y.T, 169.L.K & 826.V.M, 169.L.K & 887.T.D, 169.L.K & 891.S.Q, 169.L.Q & 171.A.D, 169.L.Q & 171.A.Y, 169.L.Q & 171.A.S, 169.L.Q & 224.G.T, 169.L.Q & 304.M.T, 169.L.Q & 398.Y.T, 169.L.Q & 826.V.M, 169.L.Q & 887.T.D, 169.L.Q & 891.S.Q, 171.A.D & 224.G.T, 171.A.D & 304.M.T, 171.A.D & 398.Y.T, 171.A.D & 826.V.M, 171.A.D & 887.T.D, 171.A.D & 891.S.Q, 171.A.Y & 224.G.T, 171.A.Y & 304.M.T, 171.A.Y & 398.Y.T, 171.A.Y & 826.V.M, 171.A.Y & 887.T.D, 171.A.Y & 891.S.Q, 171.A.S & 224.G.T, 171.A.S & 304.M.T, 171.A.S & 398.Y.T, 171.A.S & 826.V.M, 171.A.S & 887.T.D, 171.A.S & 891.S.Q, 4.I.G & 35.R.P, 224.G.T & 304.M.T, 224.G.T & 398.Y.T, 224.G.T & 826.V.M, 224.G.T & 887.T.D, 224.G.T & 891.S.Q, 5.-.G & 35.R.P, 4.I.G & 27.-.R, 304.M.T & 398.Y.T, 304.M.T & 826.V.M, 304.M.T & 887.T.D, 304.M.T & 891.S.Q, 9.K.G & 35.R.P, 5.-.G & 27.-.R, 4.I.G & 9.K.G, 398.Y.T & 826.V.M, 398.Y.T & 887.T.D, 398.Y.T & 891.S.Q, 27.-.R & 35.R.P, 9.K.G & 27.-.R, 5.-.G & 9.K.G, 4.I.G & 5.-.G, 826.V.M & 887.T.D, 826.V.M & 891.S.Q, 5.K.G & 27.-.R, 5.K.G & 169.L.K, 5.K.G & 171.A.D, 5.K.G & 304.M.T, 5.K.G & 398.YT, 5.K.G & 891.S.Q, 6.-.G & 27.-.R, 6.-.G & 169.L.K, 6.-.G & 171.A.D, 6.-.G & 304.M.T, 6.-.G & 398.Y.T, 6.-.G & 891.S.Q, 304.M.W & 27.-.R, 304.M.W & 169.L.K, 304.M.W & 171.A.D, 304.M.W & 398.Y.T, 304.M.W & 891.S.Q, 481.E.D & 27.-.R, 481.E.D & 169.L.K, 481.E.D & 171.A.D, 481.E.D & 304.M.T, 481.E.D & 398.Y.T, 481.E.D & 891.S.Q, 698.S.R & 27.-.R, 698.S.R & 169.L.K, 698.S.R & 171.A.D, 698.S.R & 304.M.T, 698.S.R & 398.Y.T, and 698.S.R & 891.S.Q.

52. The engineered CasX protein of claim 42, comprising three mutations selected from the group consisting of (a) 27.-.R, 169.L.K, and 329.G.K; (b) 27.-.R, 171.A.D, and 224.G.T; and (c) 35.R.P, 171.A.Y, and 304.M.T, wherein the mutations result in an improved characteristic compared to unmodified CasX 515.

53. The engineered CasX protein of any one of claims 42-51, comprising a sequence selected from SEQ ID NOS: 24916-49628, 49746-49747, and 49871-49873, or a sequence having at least 70% sequence identity thereto.

54. The engineered CasX protein of any one of claims 42-51, comprising a sequence selected from SEQ ID NOS: 24916-49628, 49746-49747, and 49871-49873.

55. The engineered CasX protein of any one of claims 42-49, comprising a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747 and 49871-49873, or a sequence having at least about 70%, at least about 80%, at least about 90%, at least about 95%, at least about 98%, or at least about 99% sequence identity thereto.

56. The engineered CasX protein of any one of claims 42-50, comprising a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873.

57. The engineered CasX protein of any one of claims 42-56, wherein the improved characteristic is one or more of editing activity, improved editing specificity, improved specificity ratio, improved editing activity and editing specificity, or improved editing activity and improved specificity ratio.

58. The engineered CasX protein of any one of claims 42-56, wherein the engineered CasX comprises a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 or a sequence having at least about 70%, at least about 80%, at least about 90%, at least about 95%, at least about 98%, or at least about 99% sequence identity thereto, wherein the engineered CasX exhibits improved editing activity compared to unmodified CasX 515.

59. The engineered CasX protein of any one of claims 42-56, wherein the engineered CasX comprises a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873, or a sequence having at least about 70%, at least about 80%, at least about 90%, at least about 95%, at least about 98%, or at least about 99% sequence identity thereto, wherein the engineered CasX exhibits improved editing specificity compared to unmodified CasX 515.

60. The engineered CasX protein of any one of claims 42-56, wherein the engineered CasX comprises a sequence selected from the group consisting of SEQ ID NOS: 27858, 27859, 27861, 27865, 27866, 27868, 27870, 27871, 27872, 27876, 27877, 27880, 27882, 27889, 27897, 27898, 27903, 27952, 27953, 27954, 27955, 27958, 27959, 27961, 27963, 27969, 27970, 27973, 27975, 27982, 27990, 27991, 27996, 27998, 28003, 28004, 28006, 28008, 28009, 28010, 28014, 28018, 28027, 28035, 28036, 28047, 28048, 28050, 28052, 28053, 28054, 28058, 28062, 28071, 28079, 28080, 28095, 28101, 28105, 28123, 28137, 28143, 28147, 28165, 28253, 28255, 28257, 28258, 28259, 28263, 28267, 28276, 28284, 28285, 28293, 28295, 28296, 28297, 28301, 28305, 28314, 28322, 28323, 28368, 28369, 28370, 28374, 28378, 28387, 28395, 28396, 28438, 28439, 28443, 28444, 28447, 28449, 28456, 28464, 28465, 28470, 28477, 28481, 28490, 28498, 28499, 28511, 28515, 28524, 28532, 28533, 28633, 28635, 28642, 28650, 28651, 28656, 28661, 28679, 28738, 28745, 28753, 28754, 28759, 28799, 28925, 28926, 29011, 29022, 29056, 29098, 29119, 29140, 29245, 29266, 29308, 29371, 29392, 29476, 29560, 29749, 29917, 29938, 30196, 30888, 31244, 31592, 33212, 33512, 34088, 34631, 34870, 35139, 35402, 35422, 35467, 35507, 35512, 43373, 49746, 49747, and 49871-49873 or a sequence having at least about 70%, at least about 80%, at least about 90%, at least about 95%, at least about 98%, or at least about 99% sequence identity thereto, wherein the engineered CasX exhibits improved editing activity and specificity compared to unmodified CasX 515.

61. The engineered CasX protein of any one of claims 42-56, wherein the engineered CasX comprises a sequence selected from the group consisting of SEQ ID NOS: 27865, 27952, 27954, 27955, 27958, 27959, 27973, 28009, 28018, 28048, 28101, 28123, 28137, 28285, 28296, 28301, 28305, 28314, 28323, 28368, 28369, 28370, 28378, 28387, 28438, 28447, 28477, 28481, 28498, 28515, 28524, 28532, 28661, 28799, 28925, 29022, 29266, 29308, 29371, 29560, 29749, 29917, 30888, 31244, 33212, 33512, 34088, 34870, 35422, 35507, 43373, 49872, and 49873, or a sequence having at least about 70%, at least about 80%, at least about 90%, at least about 95%, at least about 98%, or at least about 99% sequence identity thereto, wherein the engineered CasX exhibits improved specificity ratio compared to unmodified CasX 515.

62. The engineered CasX protein of any one of claims 42-56, wherein the engineered CasX comprises a sequence selected from the group consisting of SEQ ID NOS: 27952, 27958, 28101, 28123, 28137, 28285, 28368, 28370, 28378, 28387, 28438, 28799, 28925, 29022, 29308, 29749, 29917, 30888, 34870, 43373, and 49873, or a sequence having at least about 70%, at least about 80%, at least about 90%, at least about 95%, at least about 98%, or at least about 99% sequence identity thereto, wherein the engineered CasX exhibits improved editing activity and improved editing specificity compared to an unmodified CasX 515.

63. The engineered CasX protein of any one of claims 42-62, wherein the improved characteristic is at least about 0.1-fold to about 10-fold improved in the in vitro assay.

64. The engineered CasX variant of any one of claims 1-56, wherein the engineered CasX protein is a catalytically inactive CasX (dCasX) protein.

65. The engineered CasX variant of claim 64, wherein the dCasX comprises a mutation at residues:
a. D672A, and/or E769A, and/or D935A corresponding to the CasX protein of SEQ ID NO:1; or
D659A, and/or E756A, and/or D922A corresponding to the CasX protein of SEQ ID NO: 2.

66. An engineered CasX protein comprising two or more mutations selected from 4.I.G & 64.R.Q, 4.I.G & 169.L.K, 4.I.G & 169.L.Q, 4.I.G & 171.A.D, 4.I.G & 171.A.Y, 4.I.G & 171.A.S, 4.I.G & 224.G.T, 4.I.G & 304.M.T, 4.I.G & 398.Y.T, 4.I.G & 826.V.M, 41G & 887.T.D, 4.I.G & 891.S.Q, 5.-.G & 64.R.Q, 5.-.G & 169.L.K, 5.-.G & 169.L.Q, 5.-.G & 171.A.D, 5.-.G & 171.A.Y, 5.-.G & 171.A.S, 5.-.G & 224.G.T, 5.-.G & 304.M.T, 5.-.G & 398.Y.T, 5.-.G & 826.V.M, 5.-.G & 887.T.D, 5.-.G & 891.S.Q, 9.K.G & 64.R.Q, 9.K.G & 169.L.K, 9.K.G & 169.L.Q, 9.K.G & 171.A.D, 9.K.G & 171.A.Y, 9.K.G & 171.A.S, 9.K.G & 224.G.T, 9.K.G & 304.M.T, 9.K.G & 398.Y.T, 9.K.G & 826.V.M, 9.K.G & 887.T.D, 9.K.G & 891.S.Q, 27.-.R & 64.R.Q, 27.-.R & 169.L.K, 27.-.R & 169.L.Q, 27.-.R & 171.A.D, 27.-.R & 171.A.Y, 27.-.R & 171.A.S, 27.-.R & 224.G.T, 27.-.R & 304.M.T, 27.-.R & 398.Y.T, 27.-.R & 826.V.M, 27.-.R & 887.T.D, 27.-.R & 891.S.Q, 35.R.P & 64.R.Q, 35.R.P & 169.L.K, 35.R.P & 169.L.Q, 35.R.P & 171.A.D, 35.R.P & 171.A.Y, 35.R.P & 171.A.S, 35.R.P & 224.G.T, 35.R.P & 304.M.T, 35.R.P & 398.Y.T, 35.R.P & 826.V.M, 35.R.P & 887.T.D, 35.R.P & 891.S.Q, 887.T.D & 891.S.Q, 64.R.Q & 169.L.K, 64.R.Q & 169.L.Q, 64.R.Q & 171.A.D, 64.R.Q & 171.A.Y, 64.R.Q & 171.A.S, 64.R.Q & 224.G.T, 64.R.Q & 304.M.T, 64.R.Q & 398.Y.T, 64.R.Q & 826.V.M, 64.R.Q & 887.T.D, 64.R.Q & 891.S.Q, 169.L.K & 171.A.D, 169.L.K & 171.A.Y, 169.L.K & 171.A.S, 169.L.K & 224.G.T, 169.L.K & 304.M.T, 169.L.K & 398.Y.T, 169.L.K & 826.V.M, 169.L.K & 887.T.D, 169.L.K & 891.S.Q, 169.L.Q & 171.A.D, 169.L.Q & 171.A.Y, 169.L.Q & 171.A.S, 169.L.Q & 224.G.T, 169.L.Q & 304.M.T, 169.L.Q & 398.Y.T, 169.L.Q & 826.V.M, 169.L.Q & 887.T.D, 169.L.Q & 891.S.Q, 171.A.D & 224.G.T, 171.A.D & 304.M.T, 171.A.D & 398.Y.T, 171.A.D & 826.V.M, 171.A.D & 887.T.D, 171.A.D & 891.S.Q, 171.A.Y & 224.G.T, 171.A.Y & 304.M.T, 171.A.Y & 398.Y.T, 171.A.Y & 826.V.M, 171.A.Y & 887.T.D, 171.A.Y & 891.S.Q, 171.A.S & 224.G.T, 171.A.S & 304.M.T, 171.A.S & 398.Y.T, 171.A.S & 826.V.M, 171.A.S & 887.T.D, 171.A.S & 891.S.Q, 4.I.G & 35.R.P, 224.G.T & 304.M.T, 224.G.T & 398.Y.T, 224.G.T & 826.V.M, 224.G.T & 887.T.D, 224.G.T & 891.S.Q, 5.-.G & 35.R.P, 4.I.G & 27.-.R, 304.M.T & 398.Y.T, 304.M.T & 826.V.M, 304.M.T & 887.T.D, 304.M.T & 891.S.Q, 9.K.G & 35.R.P, 5.-.G & 27.-.R, 4.I.G & 9.K.G, 398.Y.T & 826.V.M, 398.Y.T & 887.T.D, 398.Y.T & 891. S.Q, 27.-.R & 35.R.P, 9.K.G & 27.-.R, 5.-.G & 9.K.G, 4.I.G & 5.-.G, 826.V.M & 887.T.D, 826.V.M & 891.S.Q, 5.K.G & 27.-.R, 5.K.G & 169.L.K, 5.K.G & 171.A.D, 5.K.G & 304.M.T, 5.K.G & 398.YT, 5.K.G & 891.S.Q, 6.-.G & 27.-.R, 6.-.G & 169.L.K, 6.-.G & 171.A.D, 6.-.G & 304.M.T, 6.-.G & 398.Y.T, 6.-.G & 891.S.Q, 304.M.W & 27.-.R, 304.M.W & 169.L.K, 304.M.W & 171.A.D, 304.M.W & 398.Y.T, 304.M.W & 891.S.Q, 481.E.D & 27.-.R, 481.E.D & 169.L.K, 481.E.D & 171.A.D, 481.E.D & 304.M.T, 481.E.D & 398.Y.T, 481.E.D & 891.S.Q, 698.S.R & 27.-.R, 698.S.R & 169.L.K, 698.S.R & 171.A.D, 698.S.R & 304.M.T, 698.S.R & 398.Y.T, and 698.S.R & 891.S.Q.

67. An engineered CasX protein comprising:
a. an NTSB domain sequence of SEQ ID NO: 297, or a sequence having at least about 90%, or at least about 95% sequence identity thereto;
b. a RuvC-II domain sequence of SEQ ID NO: 303, or a sequence having at least about 90%, or at least about 95% sequence identity thereto; and;
c. a helical I-II domain sequence of SEQ ID NO: 298, or a sequence having at least about 90%, or at least about 95% sequence identity thereto, comprising an amino acid substitution of position G137 relative to the sequence of SEQ ID NO: 298, wherein the substituted position G137 relative to the sequence of SEQ ID NO: 298 comprises a hydrophilic amino acid residue.

68. The engineered CasX protein of claim 67, wherein the hydrophilic amino acid residue is lysine or asparagine.

69. The engineered CasX protein of claim 67, or claim 68, comprising:
a. an OBD-I domain sequence of SEQ ID NO: 295, or a sequence having at least about 90%, or at least about 95% sequence identity thereto;
b. a helical I-I domain sequence of SEQ ID NO: 296, or a sequence having at least about 90%, or at least about 95% sequence identity thereto;
c. an OBD-II domain sequence of SEQ ID NO: 300, or a sequence having at least about 90%, or at least about 95% sequence identity thereto;
d. a RuvC-I domain sequence of SEQ ID NO: 301, or a sequence having at least about 90%, or at least about 95% sequence identity thereto; and
e. a TSL domain sequence of SEQ ID NO: 302, or a sequence having at least about 90%, or at least about 95% sequence identity thereto.

70. The engineered CasX protein of any one of claims 67-69, comprising a sequence of SEQ ID NO: 266, or a sequence having at least about 70%, at least about 80%, at least about 90%, at least about 95%, at least about 98%, at least about 99% sequence identity thereto, wherein the engineered CasX has an improved characteristic of the compared to the CasX of SEQ ID NO: 228.

71. The engineered CasX protein of claim 70, wherein the improved characteristic is one or more of improved ability to utilize a greater spectrum of protospacer adjacent motif (PAM) sequences in the editing of target nucleic acid, increased nuclease activity, increased editing of target nucleic acid, improved editing specificity for the target nucleic acid, decreased off-target editing, increased percentage of a eukaryotic genome that can be efficiently edited, improved ability to form cleavage-competent RNP with an ERS, and improved stability of an RNP complex.

72. The engineered CasX protein of claim 71, wherein the improved characteristic comprises increased editing specificity of target nucleic acid relative to the editing of the sequence of SEQ ID NO: 228, wherein the increase is at least about 1.01-fold, at least about 1.5-fold, at least about 2-fold, at least about 4-fold, at least about 10-fold, at least about 20-fold, at least about 30-fold, or at least about 40-fold greater.

73. The engineered CasX protein of claim 71, wherein the improved characteristic comprises decreased off-target editing relative to the off-target editing of the sequence of SEQ ID NO: 228.

74. The engineered CasX protein of claim 73, wherein the off-target editing is less than about 5%, less than about 4%, less than 3%, less than about 2%, less than about 1%, less than about 0.5%, less than 0.1%, when measured in silico, in an in vitro cell-free assay, or in a cell-based assay.

75. The engineered CasX protein of any one of claims 42-74, comprising one or more nuclear localization signals (NLS), and, optionally, wherein the one or more NLS are linked to the engineered CasX protein or to an adjacent NLS with a linker peptide.

76. The engineered CasX protein of claim 75, wherein the NLS is selected from the group consisting of the sequences of SEQ ID NOS: 364-457 as set forth in Table 8.

77. The engineered CasX protein of claim 75 or claim 76, wherein the linker peptide is selected from the group consisting of SR, RS, and peptides of SEQ ID NOS: 468-486.

78. The engineered CasX protein of any one of claims 75-77, wherein the one or more NLS are positioned at or near the C-terminus of the protein.

79. The engineered CasX protein of any one of claims 75-77, wherein the one or more NLS are positioned at or near at the N-terminus of the protein.

80. The engineered CasX protein of any one of claims 75-77, comprising at least two NLS, wherein the at least two NLS are positioned at or near the N-terminus and at or near the C-terminus of the protein.

81. The engineered CasX protein of any one of claims 42-80, wherein the engineered CasX protein is capable of forming a ribonuclear protein complex (RNP) with an ERS.

82. A gene editing pair comprising a ERS and an engineered CasX protein, the pair comprising an ERS of any one of claims 1-41 and an engineered CasX protein of any one of claims 42-81.

83. The gene editing pair of claim 82, wherein the ERS and the engineered CasX protein are capable of forming a ribonuclear protein complex (RNP).

84. The gene editing pair of claim 82, wherein the ERS and the engineered CasX protein are associated together as a ribonuclear protein complex (RNP).

85. The gene editing pair of any one of claims 82-84, wherein an RNP of the engineered CasX protein and the ERS exhibit at least one or more improved characteristics as compared to an RNP comprising the sequences of SEQ ID NO: 156 and SEQ ID NO: 228.

86. The gene editing pair of claim 85, wherein the improved characteristic is selected from one or more of the group consisting of increased binding affinity of the engineered CasX protein to the ERS, increased binding affinity to a target nucleic acid, increased ability to utilize a greater spectrum of one or more PAM sequences, including ATC, CTC, GTC, or TTC, in the editing of target nucleic acid, increased editing specificity of the target nucleic acid, increased nuclease activity, increased cleavage rate of the target nucleic acid, decreased off-target cleavage of the target nucleic acid, increased RNP stability, and increased ability to form cleavage-competent RNP.

87. A nucleic acid comprising a sequence that encodes the ERS of any one of claims 1-41.

88. The nucleic acid of claim 87, wherein the sequence is depleted or devoid of CpG motifs.

89. The nucleic acid of claim 88, comprising a sequence selected from the group consisting of SEQ ID NOS: 535-556.

90. A nucleic acid comprising a sequence that encodes the engineered CasX protein of any one of claims 42-81.

91. The nucleic acid of claim 88, wherein the sequence that encodes the engineered CasX protein is codon-optimized.

92. The nucleic acid of claim 91, wherein the sequence that encodes the engineered CasX protein is codon-optimized for expression in a human cell.

93. The nucleic acid of claim 90, wherein the sequence that encodes the engineered CasX protein is devoid or depleted of CpG motifs.

94. The nucleic acid of claim 93, comprising a sequence selected from the group consisting of SEQ ID NOS: 49850-49861.

95. The nucleic acid of any one of claims 90-92, wherein the nucleic acid is messenger RNA (mRNA).

96. A vector comprising:
a. the ERS of any one of claims 1-41;
b. the engineered CasX protein of any one of claims 42-81;
c. the nucleic acid of claim 87-89;
d. the nucleic acid of any one of claims 90-95; or
e. any combination of (a)-(d).

97. The vector of claim 96, wherein the vector comprises a promoter operably linked to the nucleic acid.

98. The vector of claim 96 or claim 97, wherein the vector is selected from the group consisting of a retroviral vector, a lentiviral vector, an adenoviral vector, an adeno-associated viral (AAV) vector, a herpes simplex virus (HSV) vector, a CasX delivery particle (XDP), a plasmid, a minicircle, a nanoplasmid, a DNA vector, and an RNA vector.

99. The vector of claim 98, wherein the vector is an AAV vector.

100. The vector of claim 99, wherein the AAV vector is a serotype selected from AAV1, AAV2, AAV3, AAV4, AAV5, AAV6, AAV7, AAV8, AAV9, AAV10, AAV11, AAV12, AAV 9.45, AAV 9.61, AAV-Rh74, or AAVRh10.

101. The vector of claim 100, wherein the AAV vector comprises a transgene with inverted terminal repeat (ITR) sequences derived from AAV2.

102. The vector of claim 98, wherein the vector is a retroviral vector.

103. The vector of claim 98, wherein the vector is an XDP comprising one or more components of a gag polyprotein.

104. The vector of claim 103, wherein the XDP comprises the engineered CasX protein and the ERS associated together in an RNP.

105. The vector of claim 103 or claim 104, comprising a glycoprotein tropism factor.

106. The vector of claim 105, wherein the glycoprotein tropism factor has binding affinity for a cell surface marker of a target cell and facilitates entry of the XDP into the target cell.

107. A host cell comprising the vector of any one of claims 96-106.

108. The host cell of claim 107, wherein the host cell is selected from the group consisting of a Baby Hamster Kidney fibroblast (BHK) cell, a human embryonic kidney 293 (HEK293) cell, a human embryonic kidney 293T (HEK293 T) cell, a NS0 cell, a SP2/0 cell, a YO myeloma cell, a P3X63 mouse myeloma cell, a PER cell, a PER.C6 cell, a hybridoma cell, a NIH3T3 cell, a CV-1 (simian) in Origin with SV40 genetic material (COS) cell, a HeLa cell, a Chinese hamster ovary (CHO) cell, or a yeast cell.

109. A lipid nanoparticle (LNP) comprising:
a. the ERS of any one of claims 1-41;
b. the nucleic acid of any one of claims 87-95; or
c. a combination of (a) and (b).

110. The LNP of claim 109, wherein the LNP comprises one or more components selected from the group consisting of an ionizable lipid, a helper phospholipid, a polyethylene glycol (PEG)-modified lipid, and cholesterol or a derivative thereof.

111. The LNP of claim 109, wherein the LNP comprises an ionizable lipid, a helper phospholipid, a polyethylene glycol (PEG)-modified lipid, and cholesterol or a derivative thereof.

112. The LNP of any one of claims 109-111, wherein the LNP comprises a cationic lipid comprising a pKa of about 5 to about 8.

113. A method of modifying a target nucleic acid in a cell, comprising introducing into the cell:
a. the gene editing pair of any one of claims 82-86;
b. one or more nucleic acids encoding the gene editing pair of (a);
c. a vector comprising the nucleic acid of (b);
d. an XDP comprising the gene editing pair of (a);
e. the LNP of any one of claims 109-112; or
f. combinations of two or more of (a) to (e),
wherein the target nucleic acid of the cell targeted by the ERS is modified by the engineered CasX.

114. The method of claim 113, comprising contacting the target with a plurality of gene editing pairs comprising a first and a second, or three or four ERS comprising targeting sequences complementary to different or overlapping regions of the target nucleic acid.

115. The method of claim 113, comprising contacting the target with a plurality of nucleic acids encoding gene editing pairs comprising a first and a second, three, or four ERS comprising targeting sequences complementary to different or overlapping regions of the target nucleic acid.

116. The method of claim 113, comprising contacting the target with a plurality of XDP comprising gene editing pairs comprising a first and a second, or three, or four ERSs comprising targeting sequences complementary to different or overlapping regions of the target nucleic acid.

117. The method of claim 113, comprising contacting the target nucleic acid with the gene editing pair and introducing one or more single-stranded breaks in the target nucleic acid, wherein the modifying comprises introducing a mutation, an insertion, or a deletion in the target nucleic acid.

118. The method of any one of claims 114-117, wherein the contacting comprises binding the target nucleic acid and introducing one or more double-stranded breaks in the target nucleic acid, wherein the modifying comprises introducing a mutation, an insertion, or a deletion in the target nucleic acid.

119. The method of any one of claims 113-118, wherein the modifying corrects a mutation in the gene to wild-type or results in the ability of the cell to express a functional gene product.

120. The method of any one of claims 113-118, wherein the modifying knocks down or knocks out the gene.

121. The method of any one of claims 113-118, wherein the modifying of the cell occurs in vitro or ex vivo.

122. The method of any one of claims 113-116, wherein modifying of the cell occurs in vivo.

123. The method of any one of claims 113-122, wherein the cell is a eukaryotic cell.

124. The method of claim 123, wherein the eukaryotic cell is selected from the group consisting of a rodent cell, a mouse cell, a rat cell, a primate cell, and a non-human primate cell.

125. The method of claim 123, wherein the eukaryotic cell is a human cell.

126. The method of any one of claims 113-125, wherein the cell is selected from the group consisting of an embryonic stem cell, an induced pluripotent stem cell, a germ cell, a fibroblast, an oligodendrocyte, a glial cell, a hematopoietic stem cell, a neuron progenitor cell, a neuron, a muscle cell, a bone cell, a hepatocyte, a pancreatic cell, a retinal cell, a cancer cell, a T-cell, a B-cell, an NK cell, a fetal cardiomyocyte, a myofibroblast, a mesenchymal stem cell, an autotransplanted expanded cardiomyocyte, an adipocyte, a totipotent cell, a pluripotent cell, a blood stem cell, a myoblast, an adult stem cell, a bone marrow cell, a mesenchymal cell, a parenchymal cell, an epithelial cell, an endothelial cell, a mesothelial cell, a fibroblast cell, an osteoblast cell, a chondrocyte cell, an exogenous cell, an endogenous cell, a stem cell, a hematopoietic stem cell, a bone-marrow derived progenitor cell, a myocardial cell, a skeletal cell, a fetal cell, an undifferentiated cell, a multi-potent progenitor cell, a unipotent progenitor cell, a monocyte, a cardiac myoblast, a skeletal myoblast, a macrophage, a capillary endothelial cell, a xenogenic cell, an allogenic cell, an autologous cell, and a post-natal stem cell.

127. The method of any one of claims 122-126, wherein the modifying occurs in the cells of a subject having a mutation in an allele of a gene wherein the mutation causes a disease or disorder in the subject.

128. A composition, comprising the engineered CasX protein of any one of claims 42-81.

129. The composition of claim 128, comprising the ERS of any one of claims 1-41.

130. The composition of claim 129, wherein the CasX protein and the ERS are associated together in a ribonuclear protein complex (RNP).

131. A composition, comprising an ERS of any one of claims 1-41.

132. The composition of claim 131, comprising the engineered CasX protein of any one of claims 42-81.

133. The composition of claim 132, wherein the engineered CasX protein and the ERS are associated together in a ribonuclear protein complex (RNP).

134. The composition of any one of claims 129-133, wherein the ERS comprises a targeting sequence of 15 to 20 nucleotides, wherein the targeting sequence is complementary to a target nucleic acid.

135. The composition of claim 134, wherein the targeting sequence has 20 nucleotides.

136. A pharmaceutical composition comprising the composition of any one of claims 128-133 and a pharmaceutically acceptable excipient.

137. A pharmaceutical composition comprising the LNP of any one of claims 109-112 and a suitable container.

138. A kit comprising the pharmaceutical composition of claim 136 or claim 137 and a suitable container.

139. An engineered CasX protein comprising any one of the sequences set forth in SEQ ID NOS: 24916-49628, 49746-49747, and 49871-49873.

140. An engineered CasX protein comprising any one of the sequences listed in Table 5.

141. A ERS comprising any one of the ERS sequences selected from the group consisting of SEQ ID NOS: 11,568-22,227 and 23,572-24,915.

142. The ERS of claim 141, comprising a targeting sequence having 15-20 nucleotides, wherein the targeting sequence is complementary to a target nucleic acid.

143. The ERS of claim 142, wherein the targeting sequence has 20 nucleotides.

144. The composition of any one of claims 128-135 for use in the manufacture of a medicament for the treatment a subject having a disease.
